SEQUENCE LISTING (7 sequences)

SEQ ID NO: 1 - protein, 360 aa, Homo sapiens
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMES

SEQ ID NO: 2 - protein, 17 aa, Homo sapiens
  MISC_FEATURE (4)..(16): Xaa is any amino acid
IIHXKRXXREXXLLXXM

SEQ ID NO: 3 - protein, 6 aa, Homo sapiens
DIIHRD

SEQ ID NO: 4 - protein, 10 aa, Homo sapiens
DFGLARHTDD

SEQ ID NO: 5 - protein, 12 aa, Homo sapiens
EMTGYVATRWYR

SEQ ID NO: 6 - protein, 4 aa, Homo sapiens
WMHY

SEQ ID NO: 7 - protein, 4 aa, Homo sapiens
YGSV

(19) United States
(12) Patent Application Publication
(10) Pub. No.: US 2009/0312349 A1
(43) Pub. Date: Dec. 17, 2009

(54) ANTI-INFLAMMATORY MEDICAMENTS

(75) Inventors: Daniel L. Flynn, Lawrence, KS (US); Peter A. Petillo, Lawrence, KS (US)

Correspondence Address: WilmerHale/Deciphera Pharmaceuticals, 60 State Street, Boston, MA, 02109, US

(73) Assignee: Deciphera Pharmaceuticals, LLC

(21) Appl. No.: 11/721,026

(22) PCT Filed: Dec. 23, 2005
(86) PCT No.: PCT/US05/47597

§ 371 Date: Nov. 2, 2008

Related U.S. Application Data
(60) Provisional application No. 60/638,987, filed on Dec. 23, 2004.

(51) Int. Cl. A61K 31/519; C07D 401/12; A61K 31/4439; C07D 471/04; A61P 35/00; C12N 9/12

(52) U.S. Cl. 514/264.11; 546/275.4; 514/341; 514/264.1; 544/279; 435/194

(57) ABSTRACT

Novel compounds and methods of using those compounds for the treatment of inflammatory conditions, hyperproliferative diseases, cancer, and diseases characterized by hyper-vascularization are provided. In a preferred embodiment, modulation of the activation state of p38 kinase protein, abl kinase protein, bcr-abl kinase protein, braf kinase protein, VEGFR kinase protein, or PDGFR kinase protein comprises the step of contacting said kinase protein with the novel compounds.

### RELATED APPLICATIONS

This application is a continuation-in-part of Application S/N 10/746,460 filed Dec. 24, 2003 and application Ser. No. 10/886,329 filed Jul. 6, 2004. This prior application is incorporated by reference herein. This application also claims the benefit of provisional application entitled Enzyme Modulators for treatment of inflammatory, autoimmune, cardiovascular, and immunological diseases, Ser. No. 60/638,987 filed Dec. 23, 2004. All of the foregoing applications are incorporated by reference herein.

### BACKGROUND OF THE INVENTION

1. Field of the Invention

The present invention relates to novel compounds and methods of using those compounds to treat anti-inflammatory diseases.

2. Description of the Prior Art

Basic research has recently provided the life sciences community with an unprecedented volume of information on the human genetic code and the proteins that are produced by it. In 2001, the complete sequence of the human genome was reported (Lander, E. S. et al. Initial sequencing and analysis of the human genome. Nature (2001) 409:860; Venter, J. C. et al. The sequence of the human genome. Science (2001) 291:1304). Increasingly, the global research community is now classifying the 50,000+ proteins that are encoded by this genetic sequence, and more importantly, it is attempting to identify those proteins that are causative of major, under-treated human diseases.

Despite the wealth of information that the human genome and its proteins are providing, particularly in the area of conformational control of protein function, the methodology and strategy by which the pharmaceutical industry sets about to develop small molecule therapeutics has not significantly advanced beyond using native protein active sites for binding to small molecule therapeutic agents. These native active sites are normally used by proteins to perform essential cellular functions by binding to and processing natural substrates or tranducing signals from natural ligands. Because these native pockets are used broadly by many other proteins within protein families, drugs which interact with them are often plagued by lack of selectivity and, as a consequence, insufficient therapeutic windows to achieve maximum efficacy. Side effects and toxicities are revealed in such small molecules, either during preclinical discovery, clinical trials, or later in the marketplace. Side effects and toxicities continue to be a major reason for the high attrition rate seen within the drug development process. For the kinase protein family of proteins, interactions at these native active sites have been recently reviewed: see J. Dumas, Protein Kinase Inhibitors: Emerging Pharmacophores 1997-2001, Expert Opinioin on. Therapeutic Patents (2001) 11: 405-429; J. Dumas, Editor, New challenges in Protein Kinase Inhibition, in Current Topics in Medicinal Chemistry (2002) 2: issue 9.

It is known that proteins are flexible, and this flexibility has been reported and utilized with the discovery of the small molecules which bind to alternative, flexible active sites with proteins. For review of this topic, see Teague, Nature Reviews/Drug Discovery, Vol. 2, pp. 527-541 (2003). See also, Wu et al., Structure, Vol. 11, pp. 399-410 (2003). However these reports focus on small molecules which bind only to proteins at the protein natural active sites. Peng et al., Bio. Organic and Medicinal Chiemistry Ltrs., Vol. 13, pp. 3693-3699 (2003), and Schindler, et al., Science, Vol. 289, p. 1938 (2000) describe inhibitors of abl kinase. These inhibitors are identified in WO Publication No. 2002/034727. This class of inhibitors binds to the ATP active site while also binding in a mode that induces movement of the kinase catalytic loop. Pargellis et al., Nature Structural Biology, Vol. 9, p. 268 (2002) reported inhibitors p38 alpha-kinase also disclosed in WO Publication No. 00143384 and Regan et al., J. Medicinal Chemistry, Vol. 45, pp. 2994-3008 (2002). This class of inhibitors also interacts with the kinase at the ATP active site involving a concomitant movement of the kinase activation loop.

More recently, it has been disclosed that kinases utilize activation loops and kinase domain regulatory pockets to control their state of catalytic activity. This has been recently reviewed (see, e.g., M. Huse and J. Kuriyan, Cell (2002) 109:275).

### SUMMARY OF THE INVENTION

The present invention is broadly concerned with new compounds for use in treating inflammatory conditions, cancer, hyperproliferative diseases, diseases characterized by hyper-vascularization, and methods of treating such conditions. In more detail, the inventive compounds have the formula

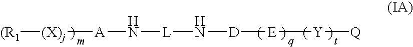

wherein:
R¹ is selected from the group consisting of aryls (preferably C₆-C₁₈, and more preferably C₆-C₁₂) and heteroaryls;
each X and Y is individually selected from the group consisting of —O—, —S—, —NR₆—, —NR₆SO₂—, —NR₆CO—, alkynyls (preferably C₁-C₁₈, and more preferably C₁-C₁₂), alkenyls (preferably C₁-C₁₈, and more preferably C₁-C₁₂), alkylenes (preferably C₁-C₁₈, and more preferably C₁-C₁₂), —O(CH₂)ₕ—, and —NR₆(CH₂)ₕ—, where each h is individually selected from the group consisting of 1, 2, 3, or 4, and where for each of alkylenes (preferably C₁-C₁₈, and more preferably C₁-C₁₂), —O(CH₂)ₕ—, and —NR₆(CH₂)ₕ—, one of the methylene groups present therein may be optionally double-bonded to a side-chain oxo group except that where —O(CH₂)ₕ— the introduction of the side-chain oxo group does not form an ester moiety;
A is selected from the group consisting of aromatic (preferably C₆-C₁₈, and more preferably C₆-C₁₂), monocycloheterocyclic, and bicycloheterocyclic rings;
D is phenyl or a five- or six-membered heterocyclic ring selected from the group consisting of pyrazolyl, pyrrolyl, imidazolyl, oxazolyl, thiazolyl, furyl, oxadiazolyl, thiadiazolyl, thienyl, pyridyl, and pyrimidyl;
E is selected from the group consisting of phenyl, pyridinyl, and pyrimidinyl;
L is selected from the group consisting of —C(O)— and —S(O)₂—;
j is 0 or 1;
k is 0 or 1;
m is 0 or 1;
n is 0 or 1;
q is 1 or 1;
t is 0 or 1;
u is 1, 2, 3, or 4;
v is 1, 2, or 3;
x is 1 or 2;
Q is selected from the group consisting of

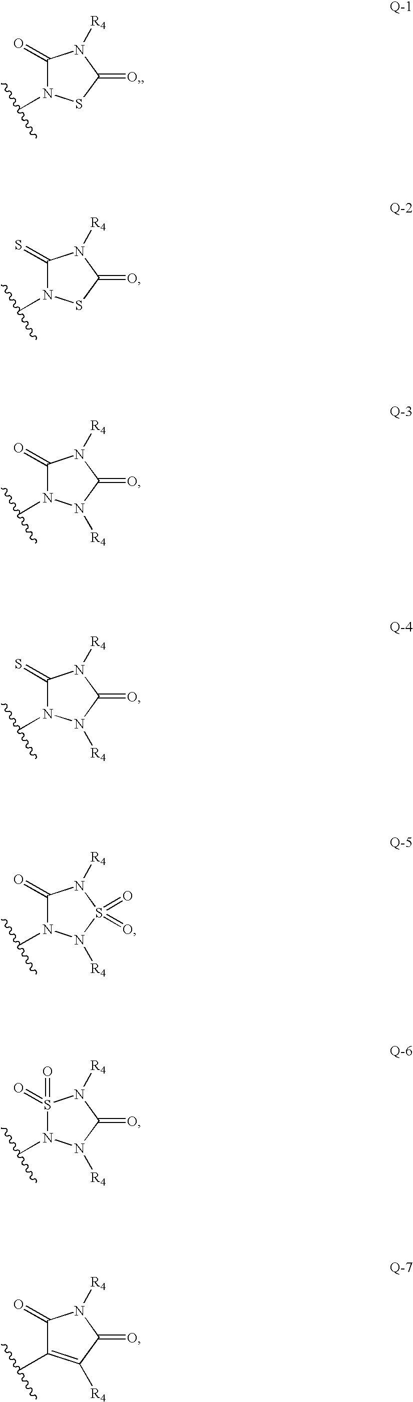

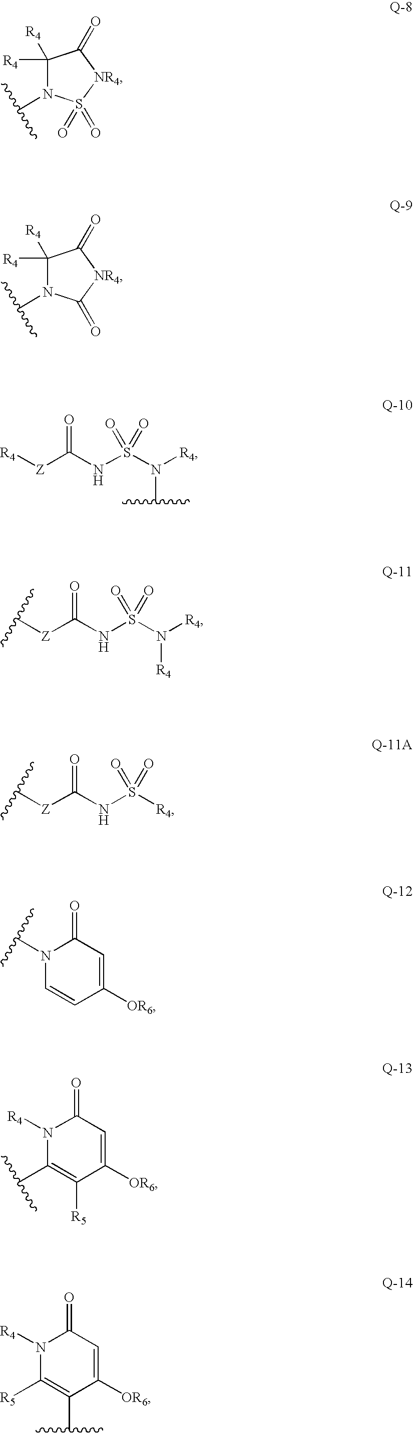

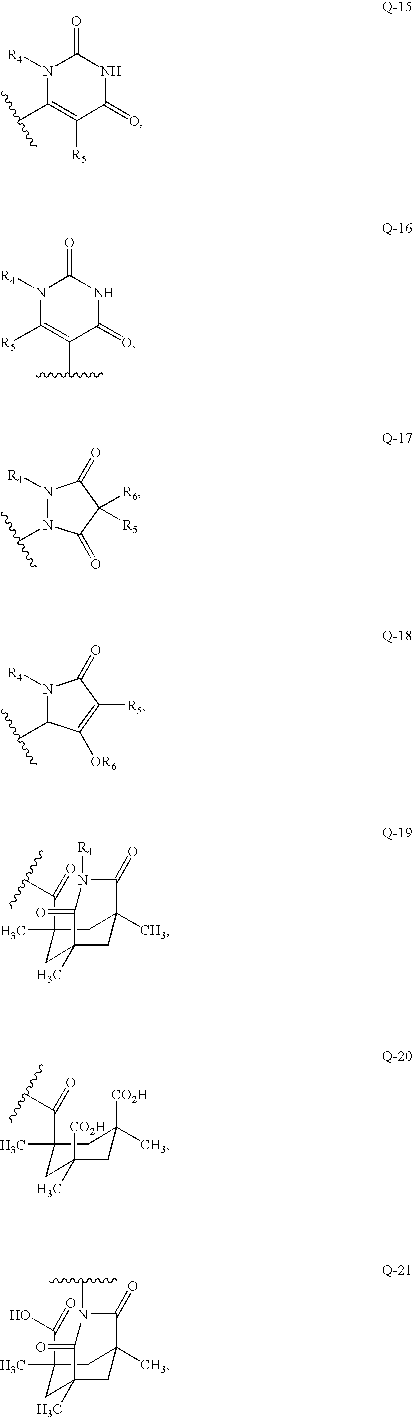

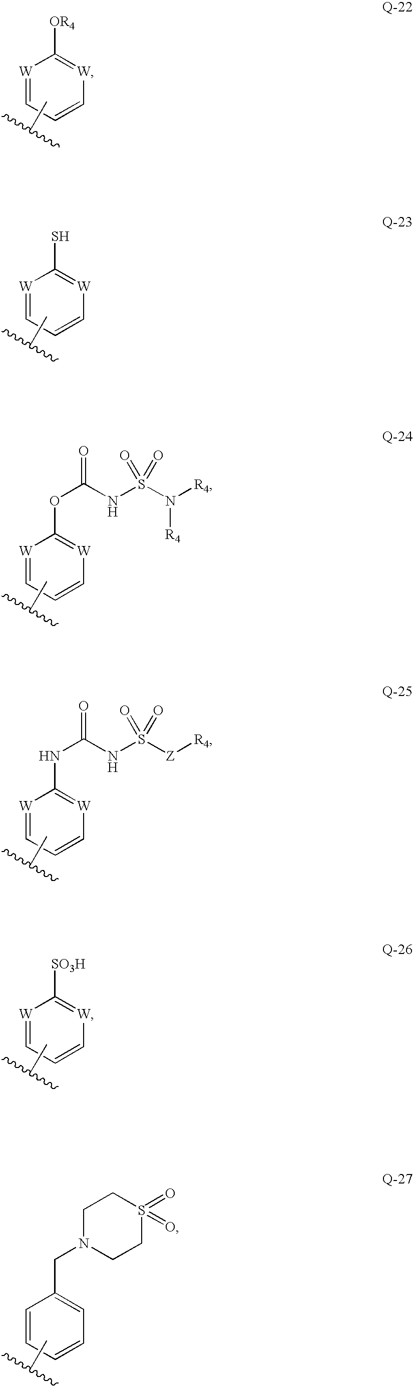

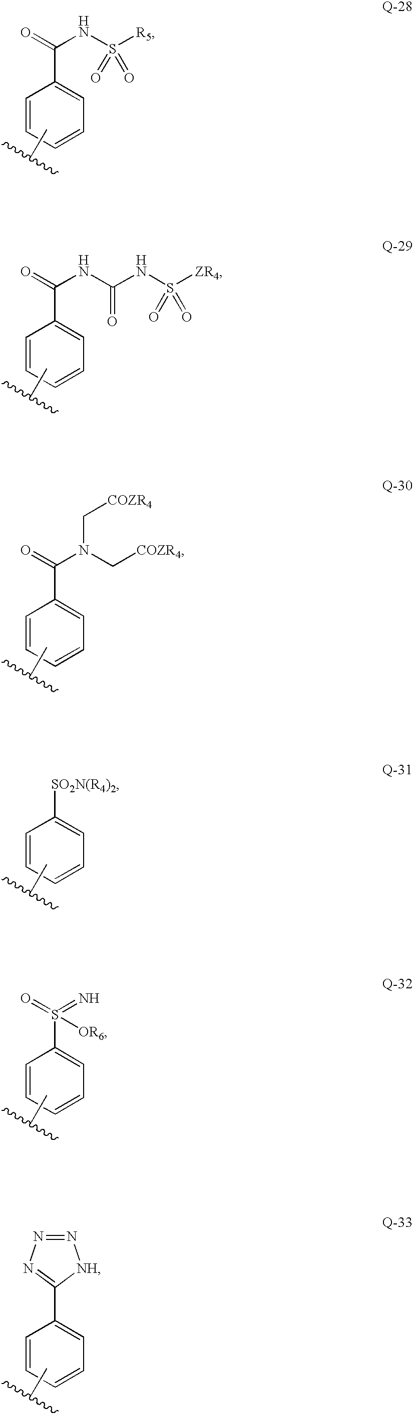

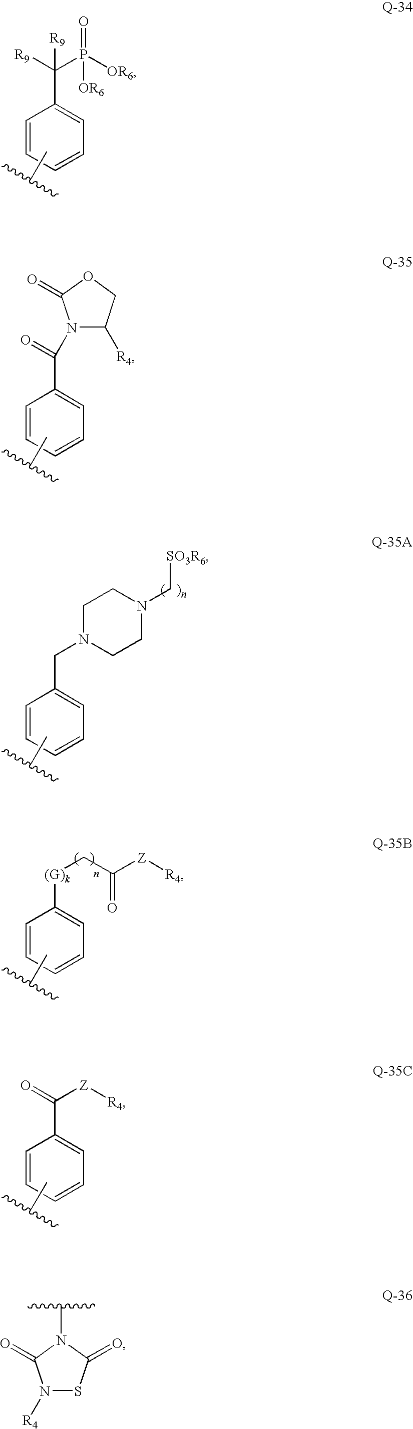

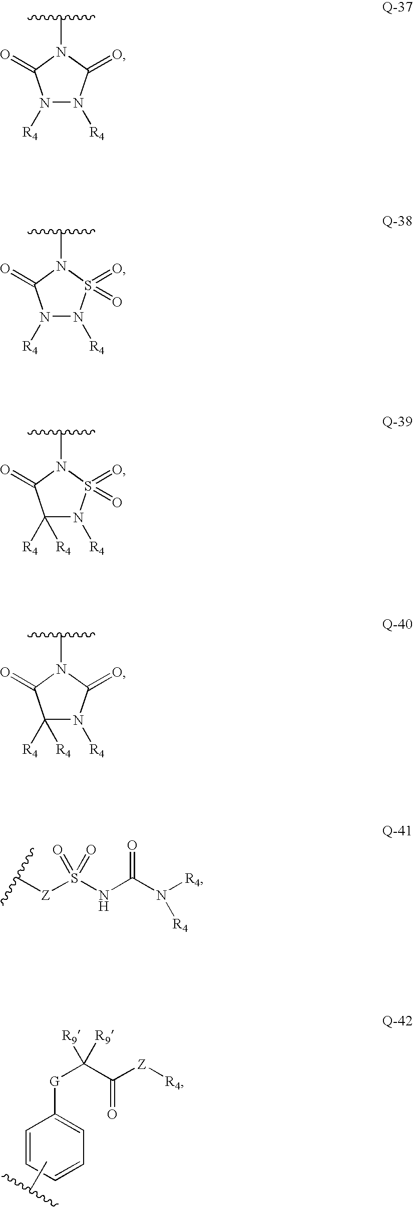

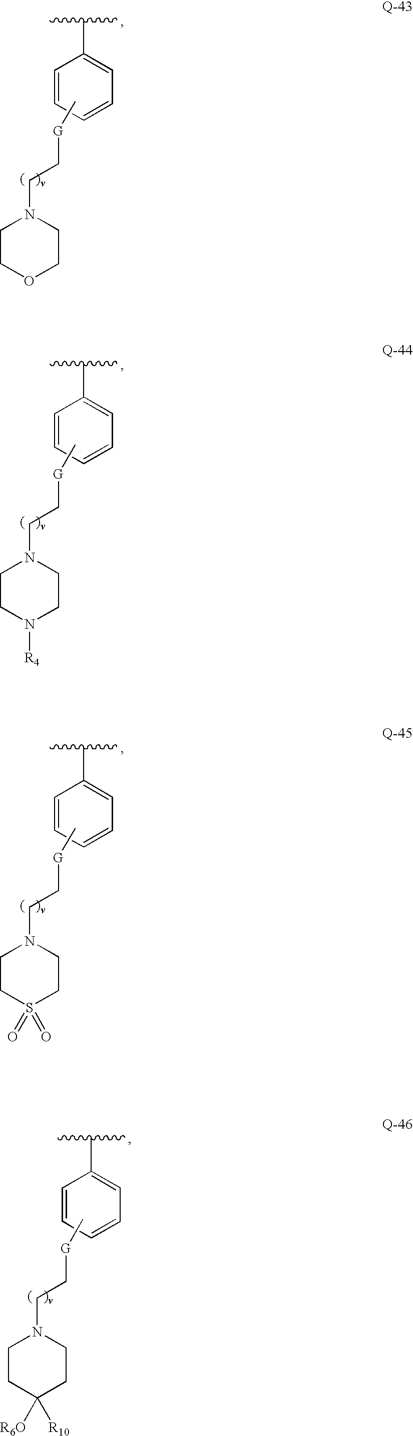

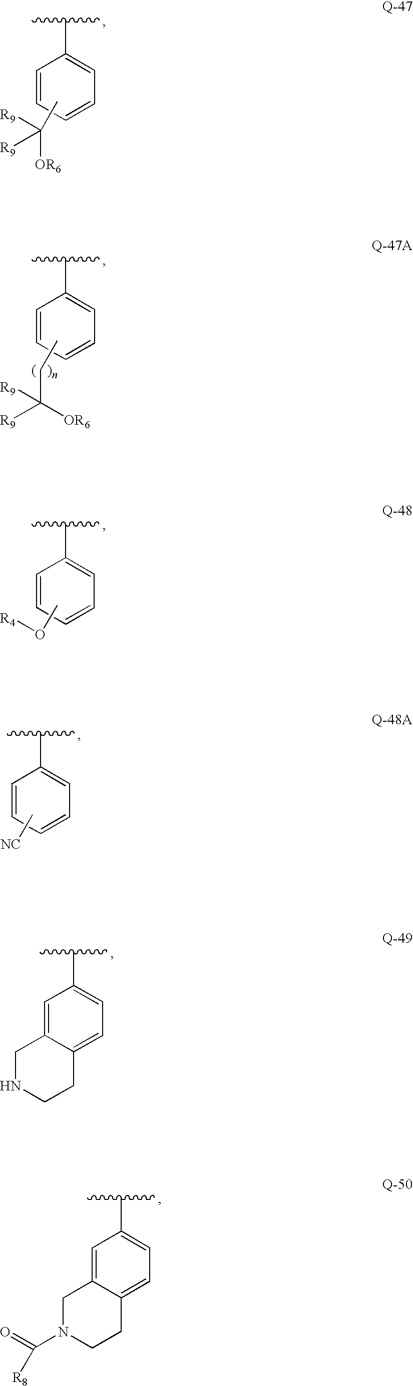

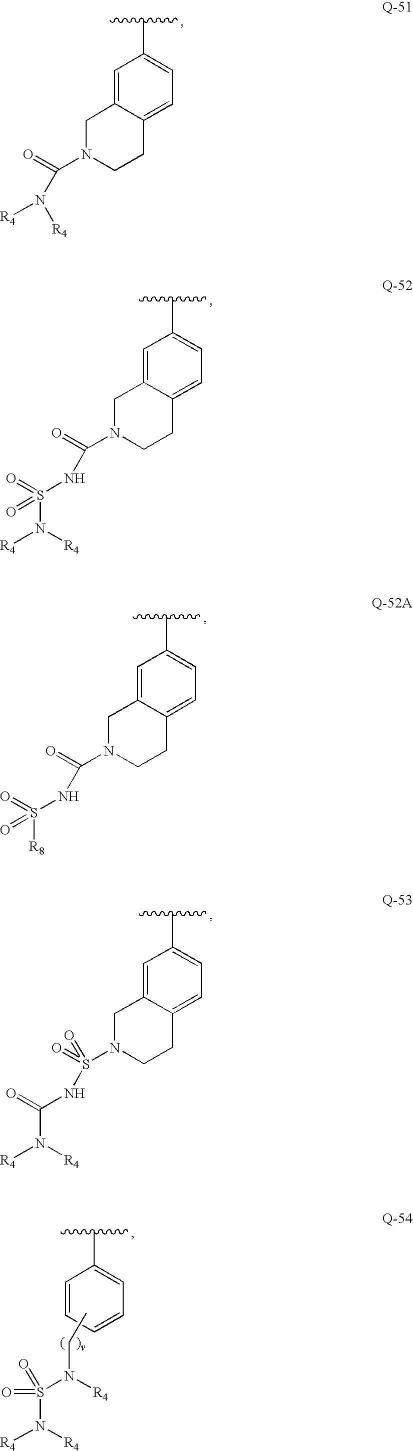

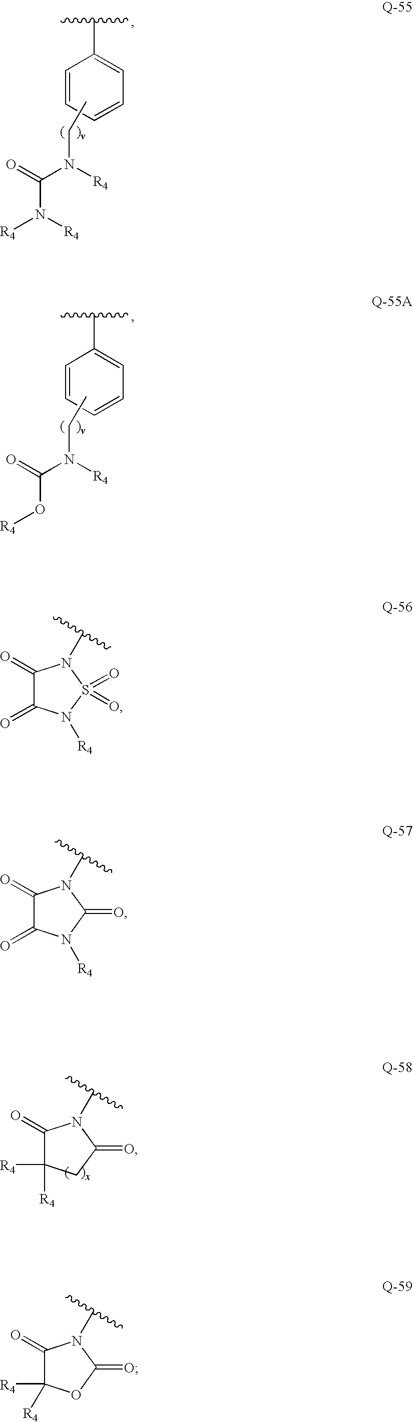

each R₄ group is individually selected from the group consisting of —H, alkyls (preferably C₁-C₁₈, and more preferably C₁-C₁₂) wherein one or more carbon atoms are optionally substituted with hydroxyl moieties, branched alkyls (preferably C₄-C₇) wherein one or more carbon atoms are optionally substituted with hydroxyl moieties, aminoalkyls (preferably C₁-C₁₈, and more preferably C₁-C₁₂), alkoxyalkyls (preferably C₁-C₁₈, and more preferably C₁-C₁₂), aryls (preferably C₆-C₁₈, and more preferably C₆-C₁₂), aralkyls (preferably C₆-C₁₈, and more preferably C₆-C₁₂ and preferably C₁-C₁₈, and more preferably C₁-C₁₂), heterocyclyls, and heterocyclylalkyls except when the R₄ constituent places a heteroatom on an alpha-carbon directly attached to a ring nitrogen on Q;
when two R₄ groups are bonded with the same atom, the two R₄ groups optionally form an alicyclic or heterocyclic 4-7 membered ring;
each R₅ is individually selected from the group consisting of —H, alkyls (preferably C₁-C₁₈, and more preferably C₁-C₁₂), aryls (preferably C₆-C₁₈, and more preferably C₆-C₁₂), heterocyclyls, alkylaminos (preferably C₁-C₁₈, and more preferably C₁-C₁₂), arylaminos (preferably C₆-C₁₈, and more preferably C₆-C₁₂), cycloalkylaminos (preferably C₁-C₁₈, and more preferably C₁-C₁₂), heterocyclylaminos, hydroxys, alkoxys (preferably C₁-C₁₈, and more preferably C₁-C₁₂), aryloxys (preferably C₆-C₁₈, and more preferably C₆-C₁₂), alkylthios (preferably C₁-C₁₈, and more preferably C₁-C₁₂), arylthios (preferably C₆-C₁₈, and more preferably C₆-C₁₂), cyanos, halogens, perfluoroalkyls (preferably C₁-C₁₈, and more preferably C₁-C₁₂), alkylcarbonyls (preferably C₁-C₁₈, and more preferably C₁-C₁₂), and nitros;
each R₆ is individually selected from the group consisting of —H, alkyls (preferably C₁-C₁₈, and more preferably C₁-C₁₂), allyls, and β-trimethylsilylethyl;
each R₈ is individually selected from the group consisting of alkyl (preferably C₁-C₁₈, and more preferably C₁-C₁₂), wherein one or more carbon atoms can be optionally substituted with a hydroxyl moiety, branched alkylC₄-C₇, wherein one or more carbon atoms can be optionally substituted with a hydroxyl moiety, phenyl, naphthyl, aralkyls (wherein the aryl is preferably C₆-C₁₈, and more preferably C₆-C₁₂, and wherein alkyl is preferably C₁-C₁₈, and more preferably C₁-C₁₂), heterocyclyls, and heterocyclylalkyls (wherein the alkyl is preferably C₁-C₁₈, and more preferably C₁-C₁₂);
each R₉ group is individually selected from the group consisting of —H, —F, alkynylC₂-C₅, alkyls (preferably C₁-C₁₈, and more preferably C₁-C₁₂), and perfluoroalkylC₁-C₃ wherein when two R₉ groups are geminal alkyl groups, said geminal alkyl groups may be cyclized to form a 3-6 membered ring;
each R₉ group is independently and individually selected from the group consisting of —H, —F, alkyl(C₁-C₆), and perfluoroalkylC₁-C₃ wherein when two R₉, groups are geminal alkyl groups, said geminal alkyl groups may be cyclized to form a 3-6 membered ring;
each R₁₀ is alkyl (preferably C₁-C₆alkyl) or fluoroalkyl (preferably C₁-C₃) wherein the fluoroalkyl moiety is partially or fully fluorinated;
G is alkylene (preferably C₁-C₈, and more preferably C₁-C₄), N(R₄), O;

### W is CH or N;

each Z is individually selected from the group consisting of —O— and —N(R₄)—; and
each ring of formula (IA) optionally includes one or more of R₇, where R₇ is a noninterfering substituent individually selected from the group consisting of —H, alkyl (preferably C₁-C₁₈, and more preferably C₁-C₁₂), aryl (preferably C₆-C₁₈, and more preferably C₆-C₁₂), heterocyclyl, alkylamino (preferably C₁-C₁₈, and more preferably C₁-C₁₂), arylamino (preferably C₆-C₁₈, and more preferably C₆-C₁₂), cycloalkylamino (preferably C₁-C₁₈, and more preferably C₁-C₁₂), heterocyclylamino, hydroxy, alkoxy (preferably C₁-C₁₈, and more preferably C₁-C₁₂), aryloxy (preferably C₆-C₁₈, and more preferably C₆-C₁₂), alkylthio (preferably C₁-C₁₈, and more preferably C₁-C₁₂), arthylthio, cyano, halogen, nitro, alkylsulfinyl (preferably C₁-C₁₈, and more preferably C₁-C₁₂), alkylsulfonyl (preferably C₁-C₁₈, and more preferably C₁-C₁₂), aminosulfonyl, alkylaminosulfonyl, dialkylaminosulfonyl, aminocarbonyl alkylaminocarbonyl, dialkylaminocarbonyl, carbonylamino, carbonylNH(alkyl), carbonylN(alkyl)₂, and perfluoroalkyl (preferably C₁-C₁₈, and more preferably C₁-C₁₂), wherein the aryl or heterocyclyl ring may optionally be further substituted by halogen, cyano, or C₁-C₃ alkyl;

As used herein, aromatic or aryl refers to monocyclic or fused bicyclic rings wherein the ring carbon atoms of at least one ring are characterized by delocalized π electrons shared among the ring carbon atoms. Such aromatic or aryl rings include phenyl, naphthyl, indenyl, or indanyl rings;

As used herein, heteroaryl, monocycloheterocyclic or monoheterocyclyl rings are taken from pyrrolyl, furyl, thienyl, oxazolyl, thiazolyl, isoxazolyl, isothiazolyl, imidazolyl, pyrazolyl, oxadiazolyl, thiadiazolyl, triazolyl, tetrazolyl, pyridinyl, pyrimidinyl, pyrazinyl, pyridazinyl, triazinyl, oxetanyl, azetadinyl, tetrahydrofuranyl, pyrrolidinyl, pyranyl, thiopyranyl, tetrahydropyranyl, dioxalinyl, piperidinyl, morpholinyl, thiomorpholinyl, piperazinyl, azepinyl, oxepinyl, and diazepinyl;

As used herein, bicycloheterocyclic or bicycloheterocyclyl rings are taken from indolyl, isoindolyl, indazolyl, benzofuranyl, benzothienyl, benzothiazolyl, benzoxazolyl, benzisoxazolyl, benzisothiazolyl, benzimidazolyl, bentriazolyl, imidazopyridinyl, purinyl, phthalimidyl, phthalimidinyl, pyrazinylpyridinyl, pyrimidinopyridinyl, pyrimidinopyrimidinyl, cinnolinyl, quinoxalinyl, quinazolinyl, quinolinyl, isoquinolinyl, phthalazinyl, benzodioxyl, indolinyl, benzisothiazoline-1,1,3-trionyl, dihydroquinolinyl, tetrahydroquinolinyl, dihydroisoquinolyl, tetrahydroisoquinolinyl, benzoazepinyl, benzodiazepinyl, benzoxapinyl, or benzoxazepinyl;

In one preferred embodiment, the compound has the structure of formula (I) except that:

when Q is Q-7, q is 0, and R₅ and D are phenyl, then A is not phenyl, oxazolyl, pyridyl, pyrimidyl, pyrazolyl, or imidazolyl;
when Q is Q-8, then Y is not —CH₂O—;
when Q is Q-10, t is 0, and E is phenyl, then any R₇ on E is not an o-alkoxy;
when Q is Q-11, t is 0, and E is phenyl, then any R₇ on E is not an o-alkoxy;
when Q is Q-22, then the compound of formula (I) is selected from the group consisting of

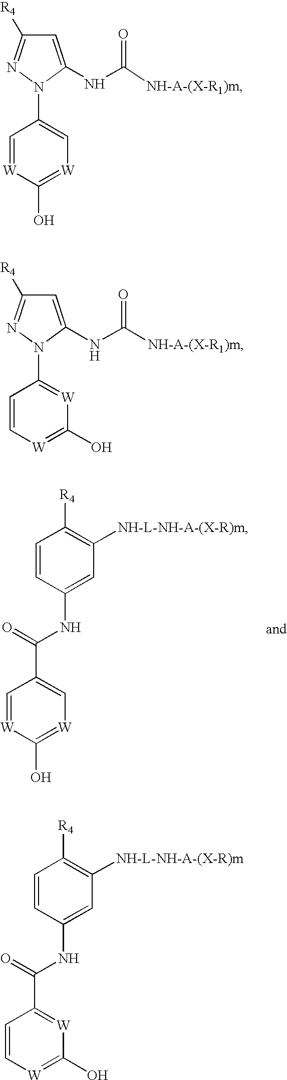

when Q is Q-24, Q-25, Q-26, or Q-31, then the compound of formula (I) is selected from the group consisting of

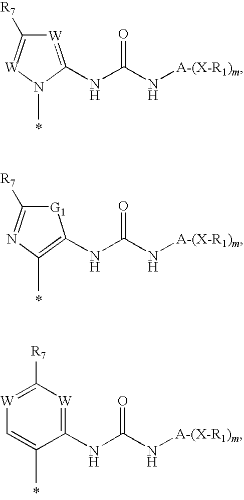

wherein each W is individually selected from the group consisting of —CH— and —N—; each G₁ is individually selected from the group consisting of —O—, —S—, and —N(R₄)—; and *denotes the point of attachment to Q-24, Q-25, Q-26, or Q-31 as follows:

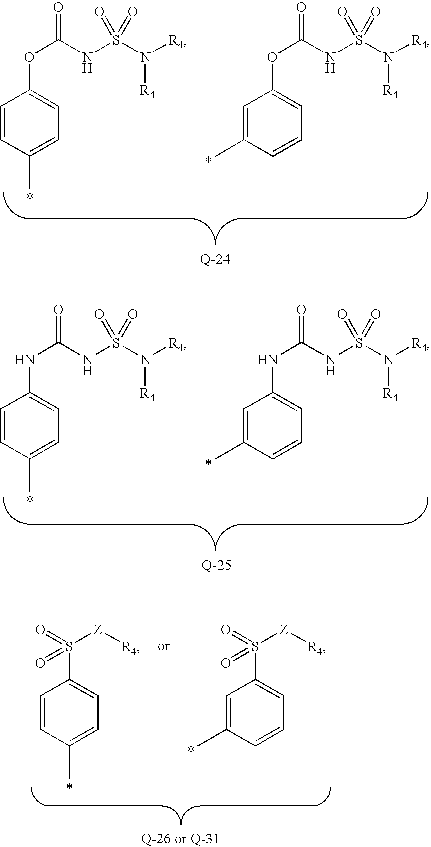

wherein each Z is individually selected from the group consisting of —O— and —N(R₄)—;
When Q is Q-35C the compound of formula I is not

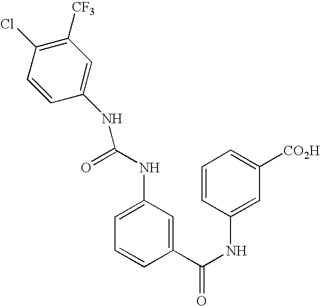

Even more preferably, R₁ as discussed above is selected from the group consisting of 6-5 fused heteroaryls, 6-5 fused heterocyclyls, 5-6 fused heteroaryls, and 5-6 fused heterocyclyls, and even more preferably, R₁ is selected from the group consisting of

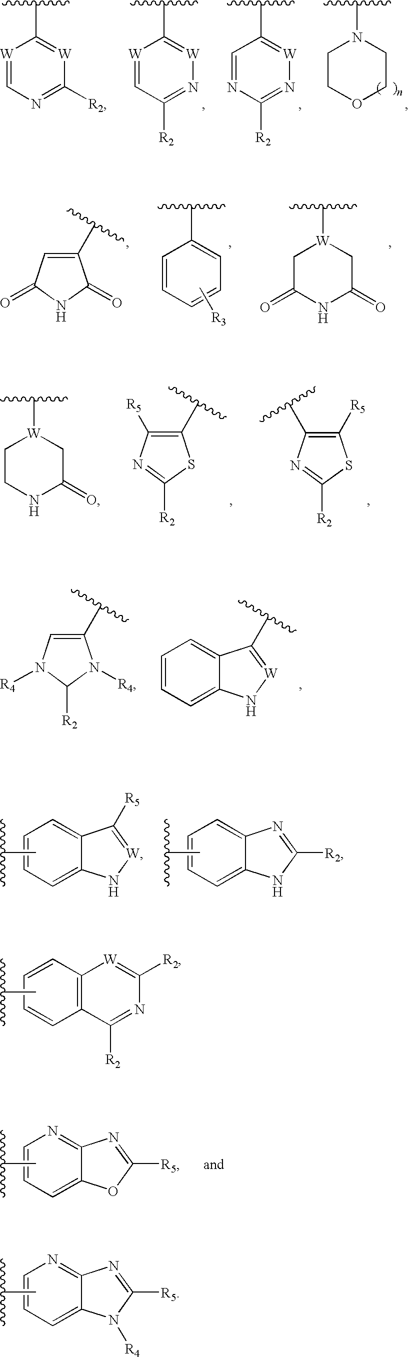

each R₂ is individually selected from the group consisting of —H, alkyls (preferably C₁-C₁₈, and more preferably C₆-C₁₂), aminos, alkylaminos (preferably C₆-C₁₈, and more preferably C₁-C₁₂), arylaminos (preferably C₆-C₁₈, and more preferably C₆-C₁₂), cycloalkylaminos (preferably C₁-C₁₈, and more preferably C₁-C₁₂), heterocyclylaminos, halogens, alkoxys (preferably C₁-C₁₈, and more preferably C₁-C₁₂), and hydroxys; and
each R₃ is individually selected from the group consisting of —H, alkyls (preferably C₁-C₁₈, and more preferably C₁-C₁₂), alkylaminos (preferably C₁-C₁₈, and more preferably C₁-C₁₂), arylaminos (preferably C₆-C₁₈, and more preferably C₆-C₁₂), cycloalkylaminos (preferably C₁-C₁₈, and more preferably C₁-C₁₂), heterocyclylaminos, alkoxys (preferably C₁-C₁₈, and more preferably C₁-C₁₂), hydroxys, cyanos, halogens, perfluoroalkyls (preferably C₁-C₁₈, and more preferably C₁-C₁₂), alkylsulfinyls (preferably C₁-C₁₈, and more preferably C₁-C₁₂), alkylsulfonyls (preferably C₁-C₁₈, and more preferably C₁-C₁₂), R₄NHSO₂—, and —NHSO₂R₂.

In another preferred embodiment, A is selected from the group consisting of aromatic, monocycloheterocyclic, and bicycloheterocyclic rings; and most preferably phenyl, naphthyl, pyridyl, pyrimidyl, thienyl, furyl, pyrrolyl, thiazolyl, isothiazolyl, oxaxolyl, isoxazolyl, imidazolyl, oxadiazolyl, thiadiazolyl, indolyl, indazolyl, benzimidazolyl, benzotriazolyl, isoquinolyl, quinolyl, benzothiazolyl, benzofuranyl, benzothienyl, pyrazolylpyrimidinyl, imidazopyrimidinyl, purinyl, and

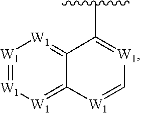

wherein each W₁ is individually selected from the group consisting of —CH— and —N—;
In another preferred embodiment, the compound of formula I is

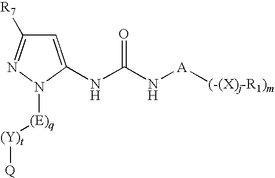

Wherein R7 is taken from the group consisting of phenyl, substituted phenyl, thienyl, and cyclopentyl;
In a further preferred embodiment, the compound of formula I is

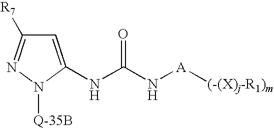

wherein q is 0, t is 0, and Q is taken from Q-35B;
and in a still more preferred embodiment, the compound of formula I is

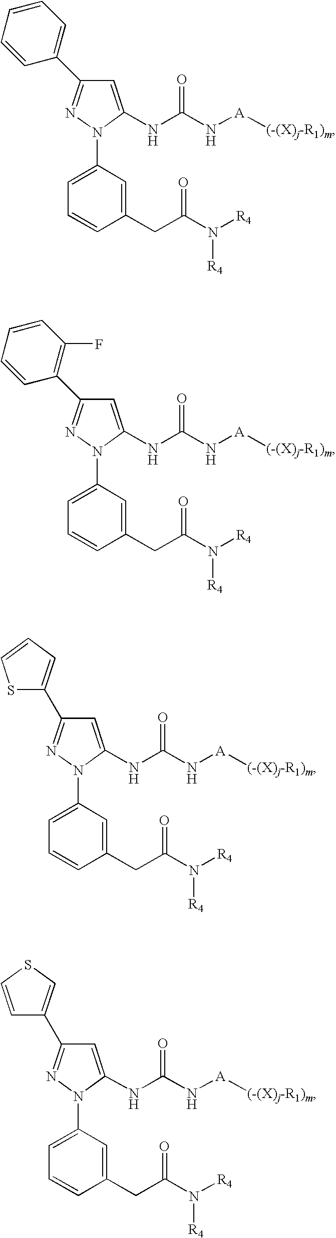

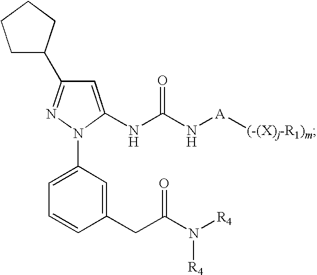

In still a further preferred embodiment, compounds of formula I are combined switch pocket modulators of kinases wherein m is 1; including compounds of the following formula

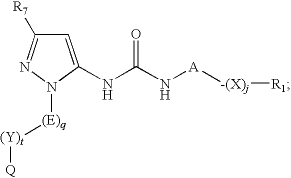

Representative examples of such combined inhibitors include

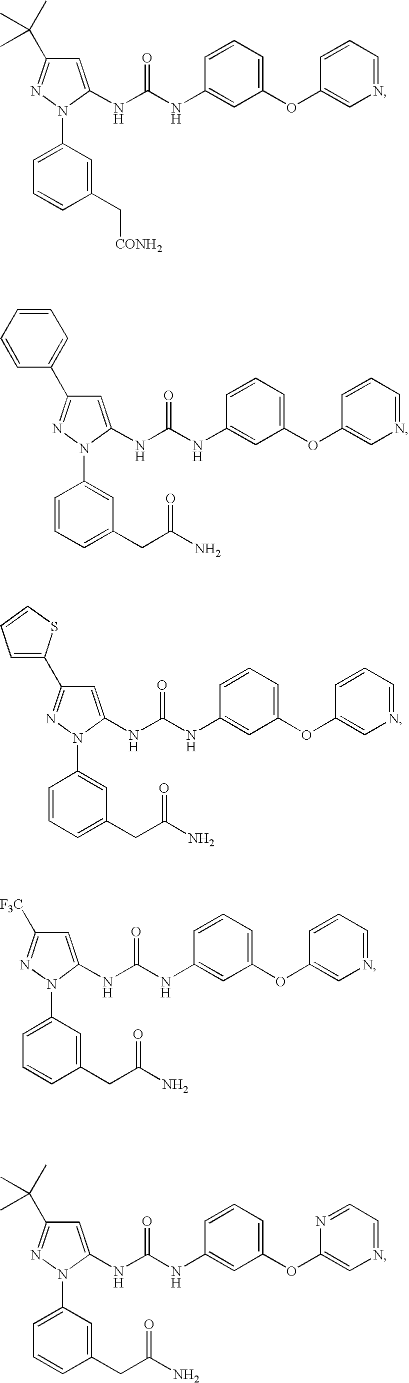

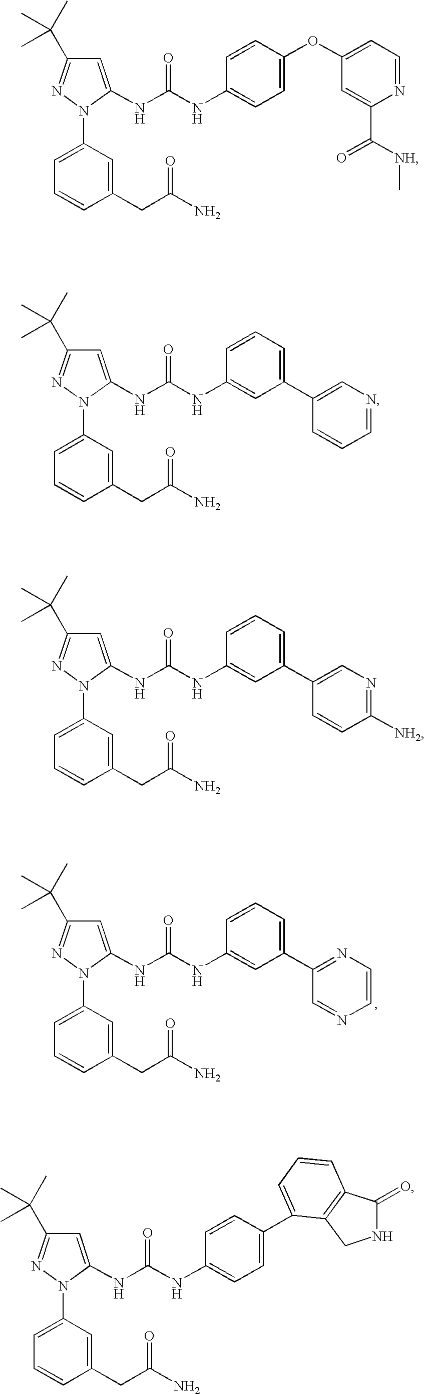

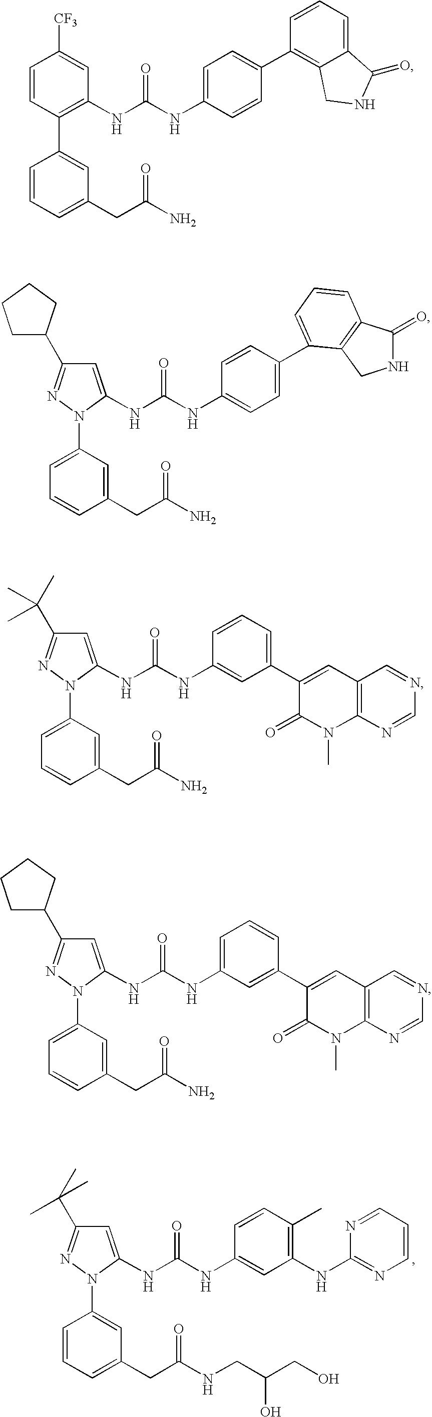

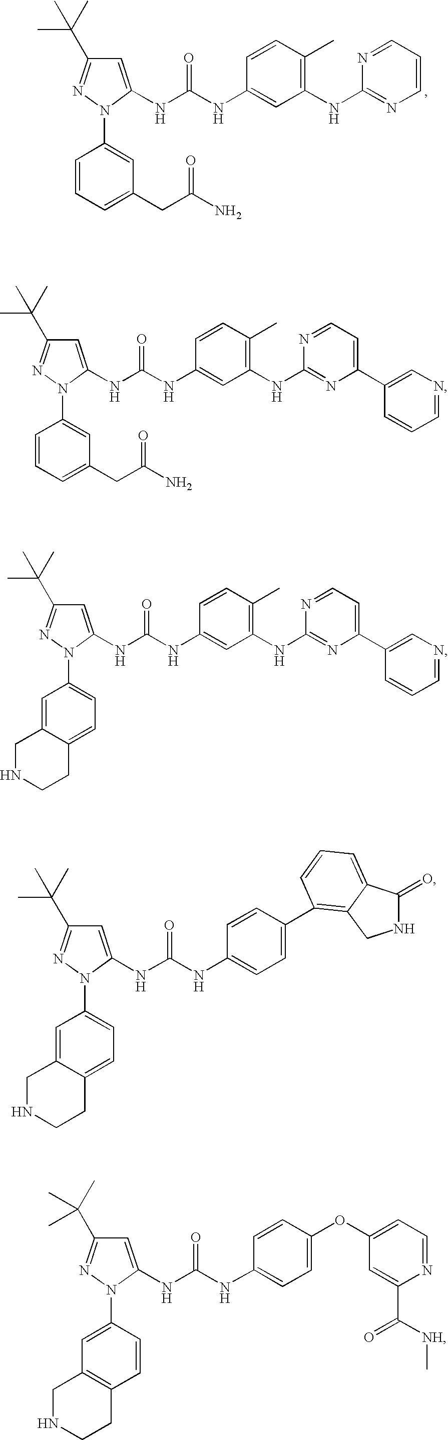

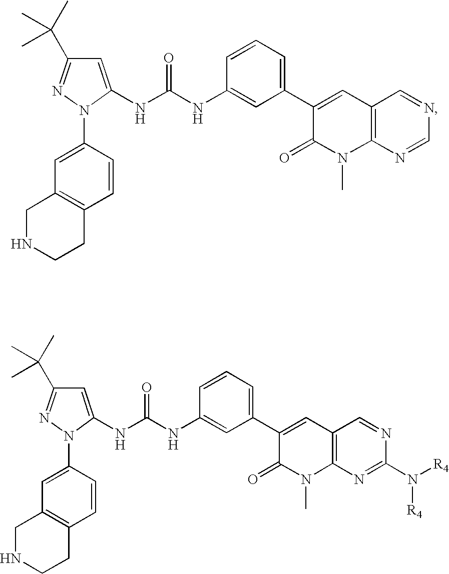

With respect to the method of using the novel compounds, the activation state of a kinase is determined by the interaction of switch control ligands and complemental switch control pockets. One conformation of the kinase may result from the switch control ligand's interaction with a particular switch control pocket while another conformation may result from the ligand's interaction with a different switch control pocket. Generally interaction of the ligand with one pocket, such as the “on” pocket, results in the kinase assuming an active conformation wherein the kinase is biologically active. Similarly, an inactive conformation (wherein the kinase is not biologically active) is assumed when the ligand interacts with another of the switch control pockets, such as the “off” pocket. The switch control pocket can be selected from the group consisting of simple, composite and combined switch control pockets. Interaction between the switch control ligand and the switch control pockets is dynamic and therefore, the ligand is not always interacting with a switch control pocket. In some instances, the ligand is not in a switch control pocket (such as occurs when the protein is changing from an active conformation to an inactive conformation). In other instances, such as when the ligand is interacting with the environment surrounding the protein in order to determine with which switch control pocket to interact, the ligand is not in a switch control pocket. Interaction of the ligand with particular switch control pockets is controlled in part by the charge status of the amino acid residues of the switch control ligand. When the ligand is in a neutral charge state, it interacts with one of the switch control pockets and when it is in a charged state, it interacts with the other of the switch control pockets. For example, the switch control ligand may have a plurality of OH groups and be in a neutral charge state. This neutral charge state results in a ligand that is more likely to interact with one of the switch control pockets through hydrogen boding between the OH groups and selected residues of the pocket, thereby resulting in whichever protein conformation results from that interaction. However, if the OH groups of the switch control ligand become charged through phosphorylation or some other means, the propensity of the ligand to interact with the other of the switch control pockets will increase and the ligand will interact with this other switch control pocket through complementary covalent binding between the negatively or positively charged residues of the pocket and ligand. This will result in the protein assuming the opposite conformation assumed when the ligand was in a neutral charge state and interacting with the other switch control pocket.

Of course, the conformation of the protein determines the activation state of the protein and can therefore play a role in protein-related diseases, processes, and conditions. For example, if a metabolic process requires a biologically active protein but the protein's switch control ligand remains in the switch control pocket (i.e. the “off” pocket) that results in a biologically inactive protein, that metabolic process cannot occur at a normal rate. Similarly, if a disease is exacerbated by a biologically active protein and the protein's switch control ligand remains in the switch control pocket (i.e. the “on” pocket) that results in the biologically active protein conformation, the disease condition will be worsened. Accordingly, as demonstrated by the present invention, selective modulation of the switch control pocket and switch control ligand by the selective administration of a molecule will play an important role in the treatment and control of protein-related diseases, processes, and conditions.

One aspect of the invention provides a method of modulating the activation state of a kinase, preferably p38α-kinase and including both the consensus wild type sequence and disease polymorphs thereof. The activation state is generally selected from an upregulated or downregulated state. The method generally comprises the step of contacting the kinase with a molecule having the general formula (I). When such contact occurs, the molecule will bind to a particular switch control pocket and the switch control ligand will have a greater propensity to interact with the other of the switch control pockets (i.e., the unoccupied one) and a lesser propensity to interact with the occupied switch control pocket. As a result, the protein will have a greater propensity to assume either an active or inactive conformation (and consequently be upregulated or downregulated), depending upon which of the switch control pockets is occupied by the molecule. Thus, contacting the kinase with a molecule modulates that protein's activation state. The molecule can act as an antagonist or an agonist of either switch control pocket. The contact between the molecule and the kinase preferably occurs at a region of a switch control pocket of the kinase and more preferably in an interlobe oxyanion pocket of the kinase. In some instances, the contact between the molecule and the pocket also results in the alteration of the conformation of other adjacent sites and pockets, such as an ATP active site. Such an alteration can also effect regulation and modulation of the active state of the protein. Preferably, the region of the switch control pocket of the kinase comprises an amino, acid residue sequence operable for binding to the Formula I molecule. Such binding can occur between the molecule and a specific region of the switch control pocket with preferred regions including the α-C helix, the α-D helix, the catalytic loop, the activation loop, and the C-terminal residues or C-lobe residues (all residues located downstream (toward the C-end) from the Activation loop), the glycine rich loop, and combinations thereof. When the binding region is the α-C helix, one preferred binding sequence in this helix is the sequence IIHXKRXXREXXLLXXM, (SEQ ID NO. 2). When the binding region is the catalytic loop, one preferred binding sequence in this loop is DIIHRD (SEQ ID NO. 3). When the binding region is the activation loop, one preferred binding sequence in this loop is a sequence selected from the group consisting of DFGLARHTDD (SEQ ID NO.4), EMTGYVATRWYR (SEQ ID NO. 5), and combinations thereof. When the binding region is in the C-lobe residues, one preferred binding sequence is WMHY (SEQ ID NO. 6). When the binding region is in the glycine rich loop one preferred binding sequence is YGSV (SEQ ID NO. 7). When a biologically inactive protein conformation is desired, molecules which interact with the switch control pocket that normally results in a biologically active protein conformation (when interacting with the switch control ligand) will be selected. Similarly, when a biologically active protein conformation is desired, molecules which interact with the switch control pocket that normally results in a biologically inactive protein conformation (when interacting with the switch control ligand) will be selected. Thus, the propensity of the protein to assume a desired conformation will be modulated by administration of the molecule. In preferred forms, the molecule will be administered to an individual undergoing treatment for a condition selected from the group consisting of human inflammation, rheumatoid arthritis, rheumatoid spondylitis, ostero-arthritis, asthma, gouty arthritis, sepsis, septic shock, endotoxic shock, Gram-negative sepsis, toxic shock syndrome, adult respiratory distress syndrome, stroke, reperfusion injury, neural trauma, neural ischemia, psoriasis, restenosis, chronic pulmonary inflammatory disease, bone resorptive diseases, graft-versus-host reaction, Chron's disease, ulcerative colitis, inflammatory bowel disease, pyresis, and combinations thereof. In such forms, it will be desired to select molecules that interact with the switch control pocket that generally leads to a biologically active protein conformation so that the protein will have the propensity to assume the biologically inactive form and thereby alleviate the condition. It is contemplated that the molecules of the present invention will be administrable in any conventional form including oral, parenteral, inhalation, and subcutaneous. It is preferred for the administration to be in the oral form. Preferred molecules include the preferred compounds of formula (I), as discussed above.

Another aspect of the present invention provides a method of treating an inflammatory condition of an individual comprising the step of administering a molecule having the general formula (I) to the individual. Such conditions are often the result of an overproduction of the biologically active form of a protein, including kinases. The administering step generally includes the step of causing said molecule to contact a kinase involved with the inflammatory process, preferably p38 α-kinase. When the contact is between the molecule and a kinase, the contact preferably occurs in an interlobe oxyanion pocket of the kinase that includes an amino acid residue sequence operable for binding to the Formula I molecule. Preferred binding regions of the interlobe oxyanion pocket include the α-C helix region, the α-D helix region, the catalytic loop, the activation loop, the C-terminal residues, the glycine rich loop residues, and combinations thereof. When the binding region is the α-C helix, one preferred binding sequence in this helix is the sequence IIHXKRXXREXXLLXXM, (SEQ ID NO. 2). When the binding region is the catalytic loop, one preferred binding sequence in this loop is DIIHRD (SEQ ID NO. 3). When the binding region is the activation loop, one preferred binding sequence in this loop is a sequence selected from the group consisting of DFGLARHTDD (SEQ ID NO.4), EMTGYVATRWYR (SEQ ID NO. 5), and combinations thereof. Such a method permits treatment of the condition by virtue of the modulation of the activation state of a kinase by contacting the kinase with a molecule that associates with the switch control pocket that normally leads to a biologically active form of the kinase when interacting with the switch control ligand. Because the ligand cannot easily interact with the switch control pocket associated with or occupied by the molecule, the ligand tends to interact with the switch control pocket leading to the biologically inactive form of the protein, with the attendant result of a decrease in the amount of biologically active protein. Preferably, the inflammatory condition is selected from the group consisting of human inflammation, rheumatoid arthritis, rheumatoid spondylitis, ostero-arthritis, asthma, gouty arthritis, sepsis, septic shock, endotoxic shock, Gram-negative sepsis, toxic shock syndrome, adult respiratory distress syndrome, stroke, reperfusion injury, neural trauma, neural ischemia, psoriasis, restenosis, chronic pulmonary inflammatory disease, bone resorptive diseases, graft-versus-host reaction, Chron's disease, ulcerative colitis, inflammatory bowel disease, pyresis, and combinations thereof. As with the other methods of the invention, the molecules may be administered in any conventional form, with any convention excipients or ingredients. However, it is preferred to administer the molecule in an oral dosage form. Preferred molecules are again selected from the group consisting of the preferred formula (I) compounds discussed above.

### BRIEF DESCRIPTION OF THE DRAWINGS

FIG. 1 is a schematic representation of a naturally occurring mammalian protein in accordance with the invention including “on” and “off” switch control pockets 102 and 104, respectively, a transiently modifiable switch control ligand 106, and an active ATP site 108;

FIG. 2 is a schematic representation of the protein of FIG. 1, wherein the switch control ligand 106 is illustrated in a binding relationship with the off switch control pocket 104, thereby causing the protein to assume a first biologically downregulated conformation;

FIG. 3 is a view similar to that of FIG. 1, but illustrating the switch control ligand 106 in its charged-modified condition wherein the OH groups 110 of certain amino acid residues have been phosphorylated;

FIG. 4 is a view similar to that of FIG. 2, but depicting the protein wherein the phosphorylated switch control ligand 106 is in a binding relationship with the on switch control pocket 102, thereby causing the protein to assume a second biologically-active conformation different than the first conformation of FIG. 2;

FIG. 4a is an enlarged schematic view illustrating a representative binding between the phosphorylated residues of the switch control ligand 106, and complemental residues Z+ from the on switch control pocket 102;

FIG. 5 is a view similar to that of FIG. 1, but illustrating in schematic form possible small molecule compounds 116 and 118 in a binding relationship with the off and on switch control pockets 104 and 102, respectively;

FIG. 6 is a schematic view of the protein in a situation where a composite switch control pocket 120 is formed with portions of the switch control ligand 106 and the on switch control pocket 102, and with a small molecule 122 in binding relationship with the composite pocket; and

FIG. 7 is a schematic view of the protein in a situation where a combined switch control pocket 124 is formed with portions of the on switch control pocket 102, the switch control ligand sequence 106, and the active ATP site 108, and with a small molecule 126 in binding relationship with the combined switch control pocket.

### DETAILED DESCRIPTION OF THE PREFERRED EMBODIMENTS

The present invention provides a way of rationally developing new small molecule modulators which interact with naturally occurring proteins (e.g., mammalian, and especially human proteins) in order to modulate the activity of the proteins. Novel protein-small molecule adducts are also provided. The invention preferably makes use of naturally occurring proteins having a conformational property whereby the proteins change their conformations in vivo with a corresponding change in protein activity. For example, a given enzyme protein in one conformation may be biologically upregulated, while in another conformation, the same protein may be biologically downregulated. The invention preferably makes use of one mechanism of conformation change utilized by naturally occurring proteins, through the interaction of what are termed “switch control ligands” and “switch control pockets” within the protein.

As used herein, “switch control ligand” means a region or domain within a naturally occurring protein and having one or more amino acid residues therein which are transiently modified in vivo between individual states by biochemical modification, typically phosphorylation, sulfation, acylation or oxidation. Similarly, “switch control pocket” means a plurality of contiguous or non-contiguous amino acid residues within a naturally occurring protein and comprising residues capable of binding in vivo with transiently modified residues of a switch control ligand in one of the individual states thereof in order to induce or restrict the conformation of the protein and thereby modulate the biological activity of the protein, and/or which is capable of binding with a non-naturally occurring switch control modulator molecule to induce or restrict a protein conformation and thereby modulate the biological activity of the protein.

A protein-modulator adduct in accordance with the invention comprises a naturally occurring protein having a switch control pocket with a non-naturally occurring molecule bound to the protein at the region of said switch control pocket, said molecule serving to at least partially regulate the biological activity of said protein by inducing or restricting the conformation of the protein. Preferably, the protein also has a corresponding switch control ligand, the ligand interacting in vivo with the pocket to regulate the conformation and biological activity of the protein such that the protein will assume a first conformation and a first biological activity upon the ligand-pocket interaction, and will assume a second, different conformation and biological activity in the absence of the ligand-pocket interaction.

The nature of the switch control ligand/switch control pocket interaction may be understood from a consideration of schematic FIGS. 1-4. Specifically, in FIG. 1, a protein 100 is illustrated in schematic form to include an “on” switch control pocket 102, and “off” switch control pocket 104, and a switch control ligand 106. In addition, the schematically depicted protein also includes an ATP active site 108. In the exemplary protein of FIG. 1, the ligand 106 has three amino acid residues with side chain OH groups 110. The off pocket 104 contains corresponding X residues 112 and the on pocket 102 has Z residues 114. In the exemplary instance, the protein 100 will change its conformation depending upon the charge status of the OH groups 110 on ligand 106, i.e., when the OH groups are unmodified, a neutral charge is presented, but when these groups are phosphorylated a negative charge is presented.

The functionality of the pockets 102, 104 and ligand 106 can be understood from a consideration of FIGS. 2-4. In FIG. 2, the ligand 106 is shown operatively interacted with the off pocket 104 such that the OH groups 110 interact with the X residues 112 forming a part of the pocket 104. Such interaction is primarily by virtue of hydrogen bonding between the OH groups 110 and the residues 112. As seen, this ligand/pocket interaction causes the protein 100 to assume a conformation different from that seen in FIG. 1 and corresponding to the off or biologically downregulated conformation of the protein.

FIG. 3 illustrates the situation where the ligand 106 has shifted from the off pocket interaction conformation of FIG. 2 and the OH groups 110 have been phosphorylated, giving a negative charge to the ligand. In this condition, the ligand has a strong propensity to interact with on pocket 102, to thereby change the protein conformation to the on or biologically upregulated state (FIG. 4). FIG. 4a illustrates that the phosphorylated groups on the ligand 106 are attracted to positively charged residues 114 to achieve an ionic-like stabilizing bond. Note that in the on conformation of FIG. 4, the protein conformation is different than the off conformation of FIG. 2, and that the ATP active site is available and the protein is functional as a kinase enzyme.

FIGS. 1-4 illustrate a simple situation where the protein exhibits discrete pockets 102 and 104 and ligand 106. However, in many cases a more complex switch control pocket pattern is observed. FIG. 6 illustrates a situation where an appropriate pocket for small molecule interaction is formed from amino acid residues taken both from ligand 106 and, for example, from pocket 102. This is termed a “composite switch control pocket” made up of residues from both the ligand 106 and a pocket, and is referred to by the numeral 120. A small molecule 122 is illustrated which interacts with the pocket 120 for protein modulation purposes.

Another more complex switch pocket is depicted in FIG. 7 wherein the pocket includes residues from on pocket 102, and ATP site 108 to create what is termed a “combined switch control pocket.” Such a combined pocket is referred to as numeral 124 and may also include residues from ligand 106. An appropriate small molecule 126 is illustrated with pocket 124 for protein modulation purposes.

It will thus be appreciated that while in the simple pocket situation of FIGS. 1-4, the small molecule will interact with the simple pocket 102 or 104, in the more complex situations of FIGS. 6 and 7 the interactive pockets are in the regions of the pockets 120 or 124. Thus, broadly the small molecules interact “at the region” of the respective switch control pocket.

### Materials and Methods

### General Synthesis of Compounds

In the synthetic schemes of this section, q is 0 or 1. When q=0, the substituent is replaced by a synthetically non-interfering group R₇.

Compounds of Formula I wherein Q is taken from Q-1 or Q-2 and Y is alkylene are prepared according to the synthetic route shown in Scheme 1.1. Reaction of isothiocyanate 1 with chlorine, followed by addition of isocyanate 2 affords 3-dxo-thiadiazolium salt 3. Quenching of the reaction with air affords compounds of Formula I-4. Alternatively, reaction of isothiocyanate 1 with isothiocyanate 5 under the reaction conditions gives rise to compounds of Formula I-7. See A. Martinez et al, Journal of Medicinal Chemistry (2002) 45: 1292.

Intermediates 1, 2 and 5 are commercially available or prepared according to Scheme 1.2. Reaction of amine 8 with phosgene or a phosgene equivalent affords isocyanate 2. Similarly, reaction of amine 8 with thiophosgene affords isothiocyanate 5. Amine 8 is prepared by palladium(0)-catalyzed amination of 9, wherein M is a group capable of oxidative insertion into palladium(0), according to methodology reported by S. Buchwald. See M. Wolter et al, Organic Letters (2002) 4:973; B. H. Yang and S. Buchwald, Journal of Organometallic Chemistry (1999) 576(1-2):125. In this reaction sequence, P is a suitable amine protecting group. Use of and removal of amine protecting groups is accomplished by methodology reported in the literature (Protective Groups in Organic Synthesis, Peter G. M. Wutts, Theodora Greene (Editors) 3rd edition (April 1999) Wiley, John & Sons, Incorporated; ISBN: 0471160199). Starting compounds 2 are commercially available or readily prepared by one of ordinary skill in the art: See March's Advanced Organic Chemistry: Reactions, Mechanisms, and Structure, Michael B. Smith & Jerry March (Editors) 5th edition (January 2001) Wiley John & Sons; ISBN: 0471585890.

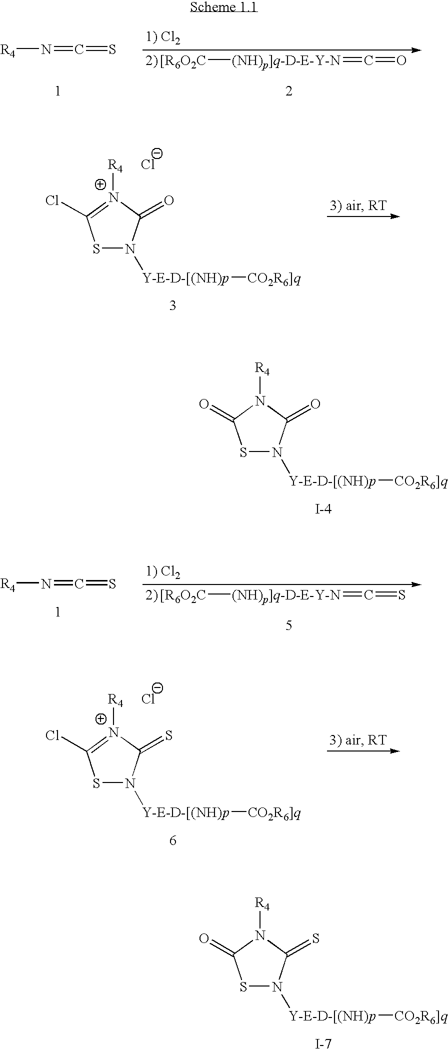

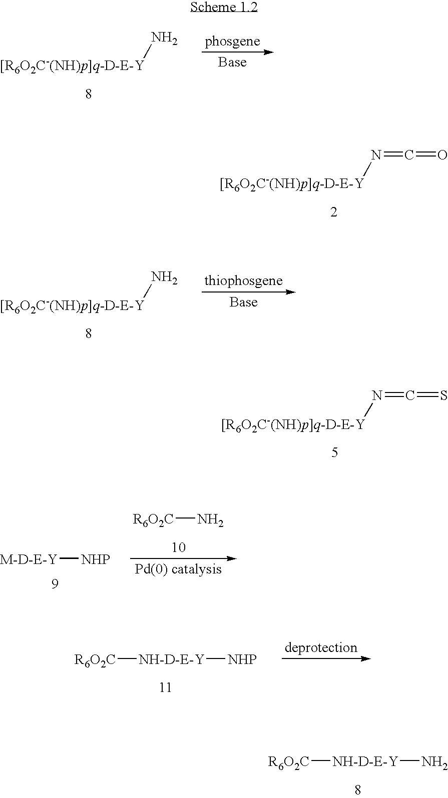

Compounds of Formula I wherein Q is taken from Q1 or Q-2 and Y is alkylene are also available via the synthetic route shown in Scheme 1.3. Reaction of amine 8 with isocyanate or isothiocyanate 2a yields the urea/thiourea 8a which can be cyclized by the addition of chlorocarbonyl sulfenyl chloride. See GB1115350 and U.S. Pat. No. 3,818,024, Revankar et. al U.S. Pat. No. 4,093,624, and Klayman et. al JOC 1972, 37(10), 1532 for further details. Where R₄ is a readily removable protecting group (e.g. R=3,4d-methoxybenzyl amine), the action of mild, acidic deprotection conditions such as CAN or TFA will reveal the parent ring system of I-4 (X═O) and I-7 (X═S).

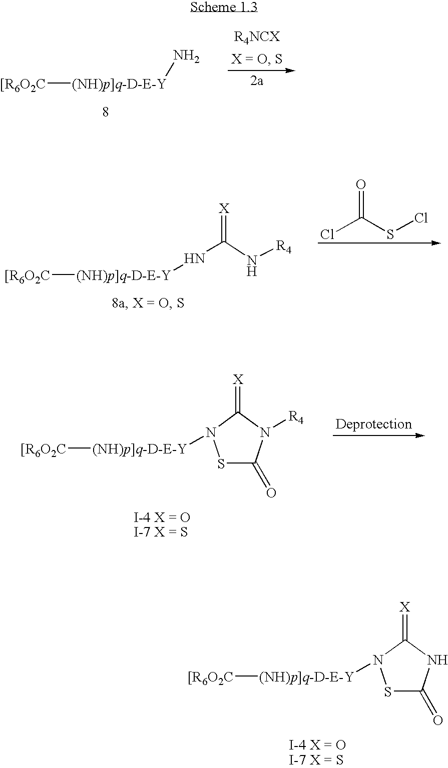

I-7 is also available as shown in Scheme 1.4. Condensation of isocyanate or isothiocyanate 2a with amine R₅NH₂ yields urea/thiourea 2b, which, when reacted with chlorocarbonyl sulfenyl chloride according to GB1115350 and U.S. Pat. No. 3,818,024 yields 2c. Where R₄ is a readily removable protecting group (e.g. R=3,4-d-methoxybenzyl amine), the action of mild, acidic deprotection conditions such as CAN or TFA will reveal the parent ring system of 2d. Reaction of 2d with NaH in DMF, and displacement wherein M is a suitable leaving group such as chloride, bromide or iodide yields I-4 (X═O) and I-7 (X═S).

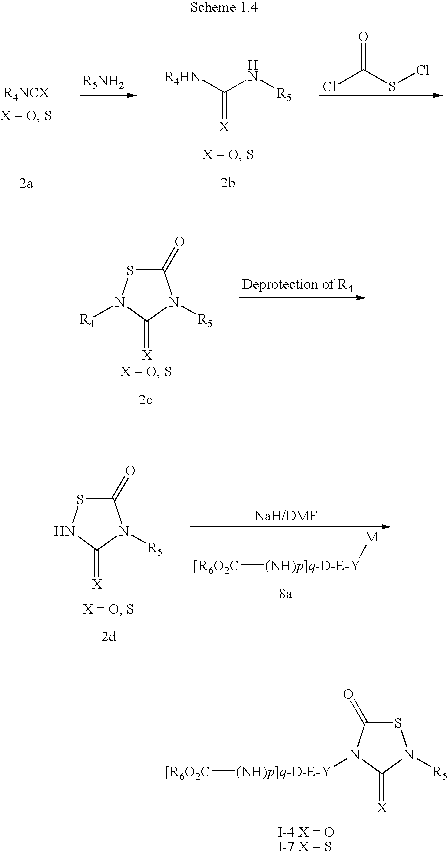

Compounds of Formula I wherein Q is taken from Q-36 and Y is alkylene are available via the synthetic route shown in Scheme 1.3. Condensation of isocyanate or isothiocyanate 2a with ammonia yields urea/thiourea 2e, which, when reacted with chlorocarbonyl sulfenyl chloride according to GB1115350 and U.S. Pat. No. 3,818,024 yields 2r. Reaction of 2f with NaH in DMF, and displacement wherein M is a suitable leaving group such as chloride, bromide or iodide yields yields I-4′ (X═O) and I-7′ (X═S).

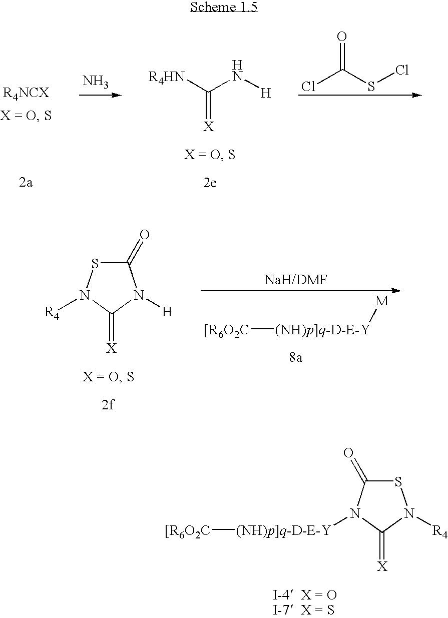

Compounds of Formula I wherein Q is taken from Q-3 or Q-4 and Y is alkylene, are prepared according to the synthetic route shown in Schemes 2.1 and 2.2, respectively. Reaction of 12, wherein M is a suitable leaving group, with the carbamate-protected hydrazine 13 affords intermediate 14. Reaction of 14 with an isocyanate gives rise to intermediate 15. Thermal cyclization of 15 affords 1,2,4-triazolidinedione of Formula I-16. By analogy, scheme 2.2 illustrates the preparation of 3-thio-5-oxo-1,2,4-triazolidines of Formula I-18 by reaction of intermediate 14 with an isothiocyanate and subsequent thermal cyclization.

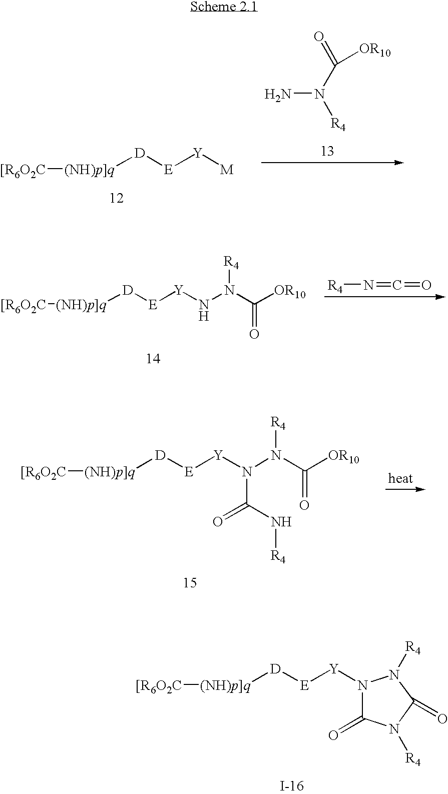

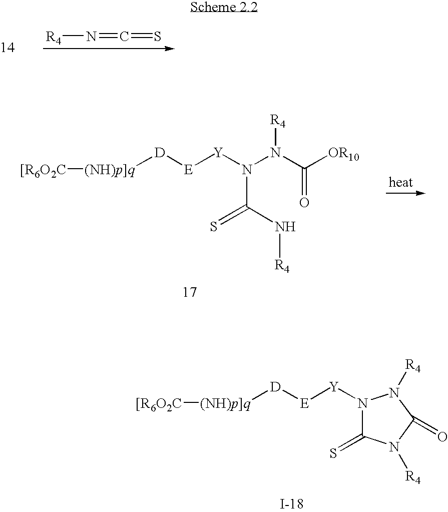

Intermediates 12 wherein p is 1 are readily available or are prepared by reaction of 19 with carbamates 10 under palladium(0)-catalyzed conditions. M₁ is a group which oxidatively inserts palladium(0), preferably iodo or bromo, and is of greater reactivity than M. Compounds 19 are either commercially available or prepared by one of ordinary skill in the art.

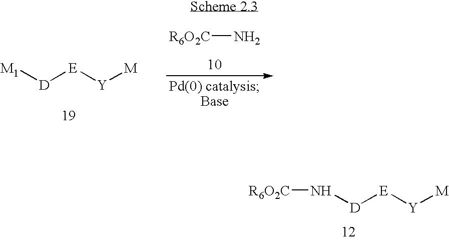

Compounds of Formula I wherein Q is taken from Q-37 and Y is alkylene, are also prepared according to the synthetic route shown in Scheme 2.4. Oxidation of amine R₄NH₂ to the corresponding hydrazine, condensation with ethyl chloroformate subsequent heating yields 1,2,4-triazolidinedione 15a. After the action of NaH in DMF, displacement wherein M is a suitable leaving group such as chloride, bromide or iodide yields I-16 (X═O) and I-18 (X═S).

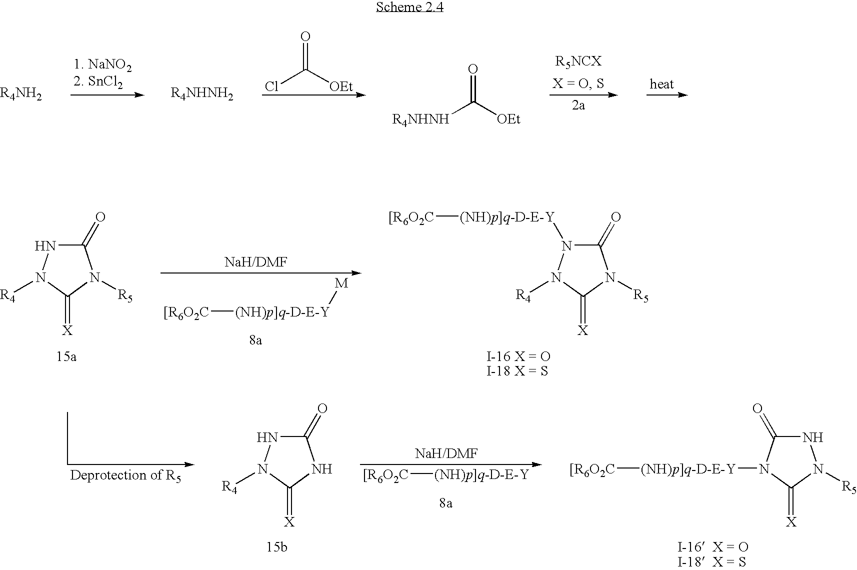

Compounds of Formula I wherein Q is taken from Q-37 and Y is alkylene, are also prepared according to the synthetic route shown in Scheme 2.4. When R₅ is a readily removable protecting group (e.g. R=3,4-d-methoxybenzyl amine), the action of mild, acidic deprotection conditions such as CAN or TFA on 15a will reveal 1,2,4-triazolidinedione 15b. After deprotonation of 15b by NaH in DMF, displacement wherein M is a suitable leaving group such as chloride, bromide or iodide yields I-16′ (X═O) and I-18′ (X═S).

Compounds of Formula I wherein Q is taken from Q-5 or Q-6 and Y is alkylene are prepared according to the synthetic route shown in Scheme 3. Reaction of hydrazine 20 with chlorosulfonylisocyanate and base, such as triethylamine, gives rise to a mixture of intermediates 21A and 21B which are not isolated but undergo cyclization in situ to afford compounds of Formulae I-22A and I-22B. Compounds I-22A and I-22B are separated by chromatography or fractional crystallization. Optionally, compounds I-22A and I-22B can undergo Mitsunobu reaction with alcohols R₄OH to give compounds of Formulae I-23A and I-23B. Compounds 20 are prepared by acid-catalyzed deprotection of t-butyl carbamates of structure 14, wherein R₁₀ is t-butyl.

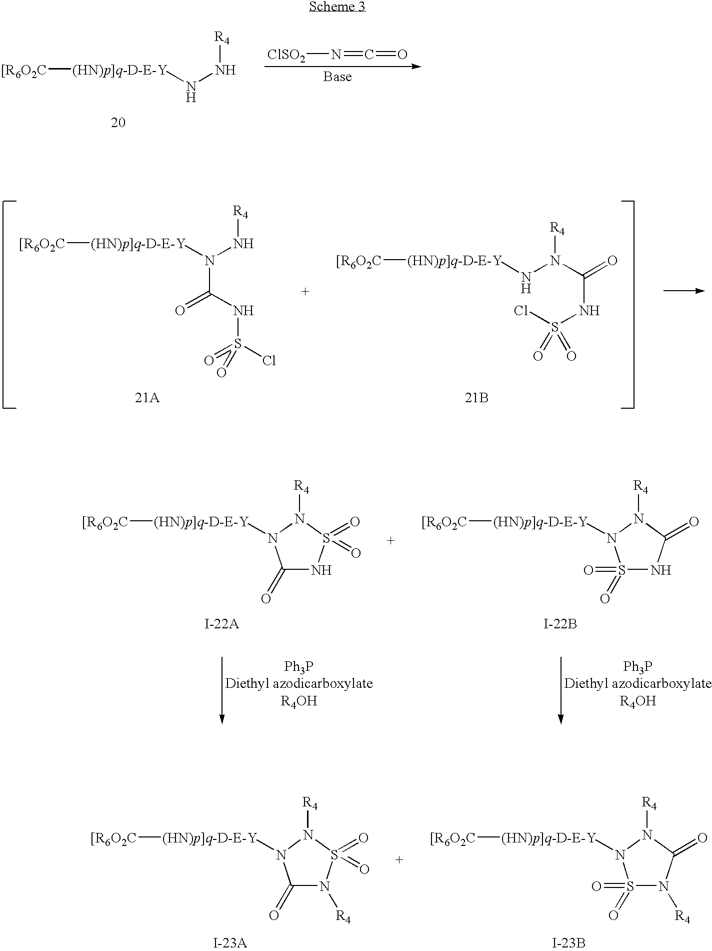

Compounds of Formula I wherein Q is Q-7 and Y is alkylene are prepared as shown in Scheme 4. Reaction of amine 8 with maleimide 24, wherein M is a suitable leaving group, affords compounds of Formula I-25. Reaction of compound 26, wherein M is a group which can oxidatively insert Pd(0), can participate in a Heck reaction with maleimide 27, affording compounds of Formula I-28. Maleimides 24 and 27 are commercially available or prepared by one of ordinary skill in the art.

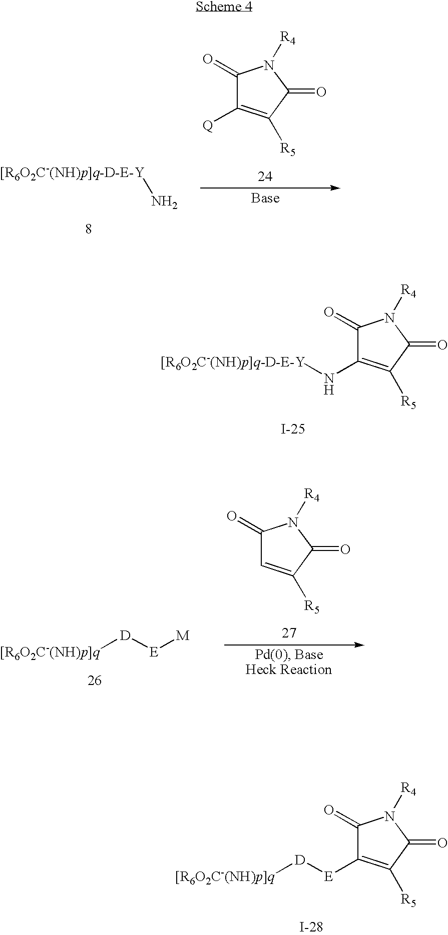

Compounds of Formula I wherein Q is Q-8 and Y is alkylene are prepared as shown in Scheme 5, according to methods reported by M. Tremblay et al, Journal of Combinatorial Chemistry (2002) 4:429. Reaction of polymer-bound activated ester 29 (polymer linkage is oxime activated-ester) with chlorosulfonylisocyante and t-butanol affords N—BOC sulfonylurea 30. Subjection of 30 to the Mitsunobu reaction with R₄OH gives rise to 31. BOC-group removal with acid, preferably trifluoroacetic acid, and then treatment with base, preferably triethylamine, provides the desired sulfahydantoin I-32. Optionally, intermediate is treated with acid, preferably trifluoroacetic acid, to afford the N-unsubstituted sulfahydantoin I-33.

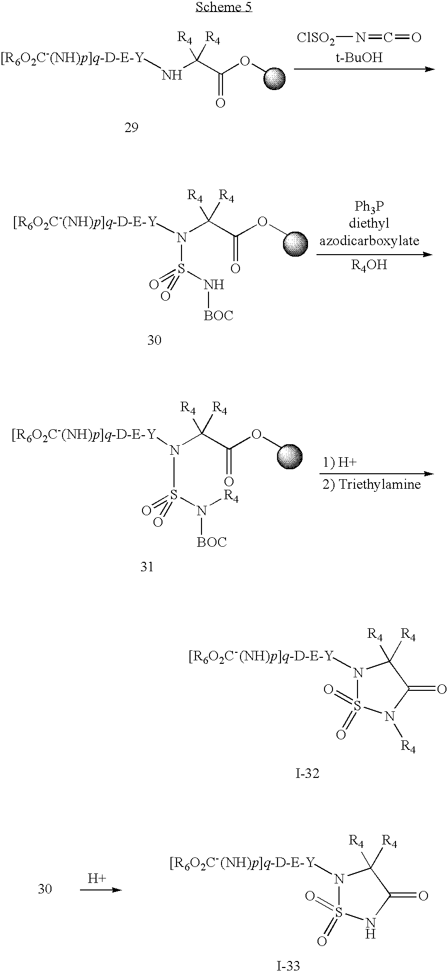

Compounds of Formula I wherein Q is Q-8 and Y is alkylene are also prepared as shown in Scheme 5a. Amine 8 is condensed with the glyoxal hemiester to yield 31a. Reaction of chlorosulphonyl isocyanate first with benzyl alcohol then 31a yields 31b, which after heating yields I-32.

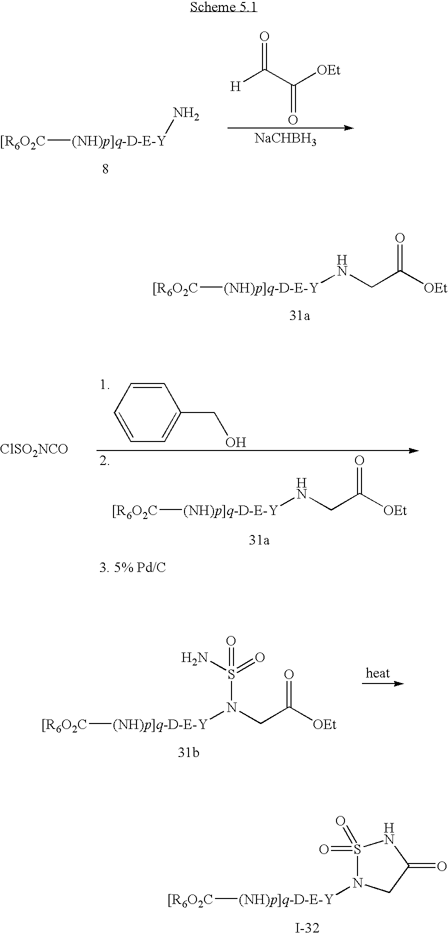

Compounds of Formula I wherein Q is taken from Q39 are prepared according to the synthetic route shown in Scheme 5.2. Formation of 31c by the method of Muller and DuBois JOC 1989, 54, 4471 and its deprotonation with NaH/DMF or NaH/DMF and subsequently alkylation wherein M is a suitable leaving group such as chloride, bromide or iodide yields I-32′. Alternatively, I-32′ is also available as shown in Scheme 5.3. Mitsunobu reaction of boc-sulfamide amino ethyl ester with alcohol 8b (made by methods analogous to that for amine 8) yields 31c, which after Boc removal with 2N HCl in dioxane is cyclized by the action of NaH on 31d results in I-32′.

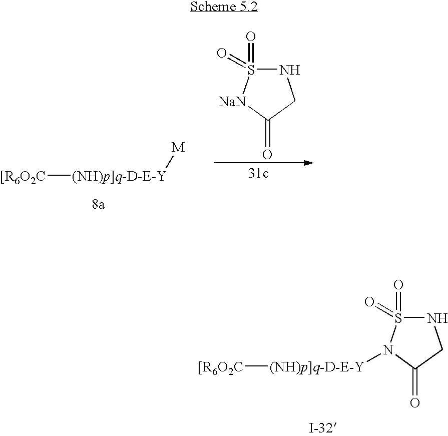

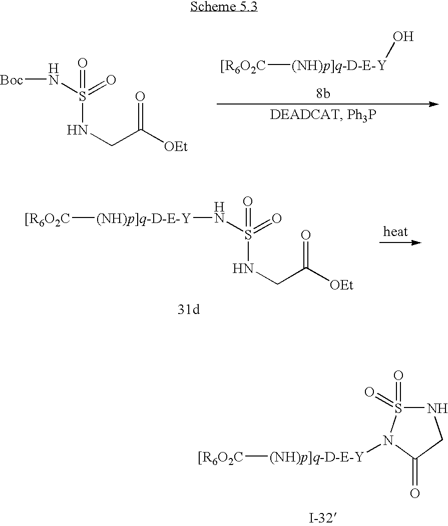

Compounds of Formula I wherein Q is Q-9 and Y is alkylene are prepared as shown in Scheme 6. Reaction of polymer-bound amino acid ester 34 with an isocyanate affords intermediate urea 35. Treatment of 35 with base, preferably pyridine or triethylamine, with optional heating, gives rise to compounds of Formula I-36.

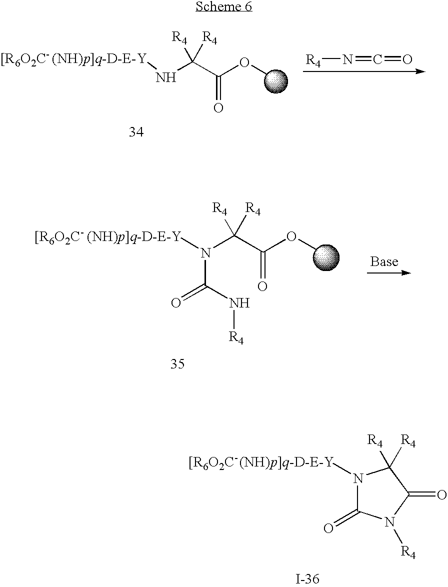

Compounds of Formula I wherein Q is Q-9 and Y is alkylene are also prepared as shown in Scheme 6.1. Reaction of aldehyde 8c under reductive amination conditions with the t-butyl ester of glycine yields 35a. Isocyanate 2a is condensed with p-nitrophenol (or the corresponding R₄NH₂ amine is condensed with p-nitrophenyl chloroformate) to yield the carbamic acid p-nitrophenyl ester, which when reacted with deprotonated 35a and yields the urea that when deprotected with acid yields 35b. Formula I-36 is directly available from 35b by the action of NaH and heat.

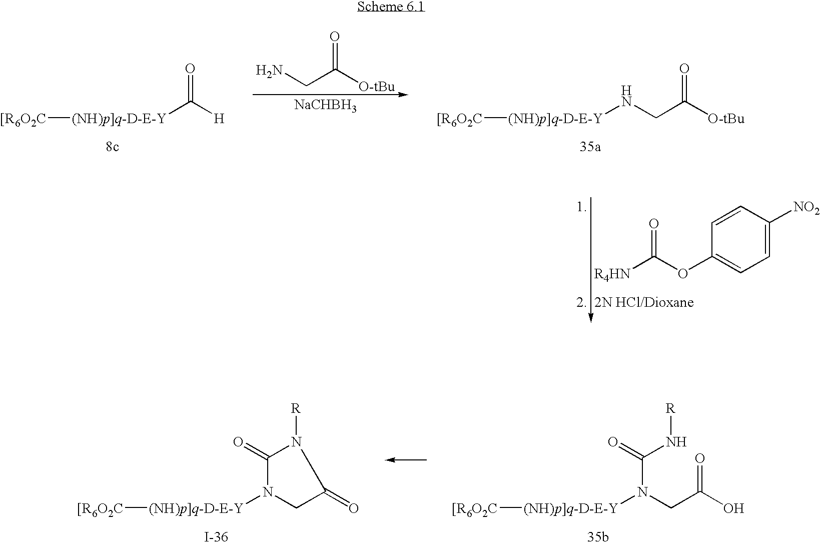

Compounds of Formula I wherein Q is taken from Q-40 are prepared according to the synthetic route shown in Scheme 6.2. Formation of 35c by the method described in JP10007804A2 and Zvilichovsky and Zucker, Israel Journal of Chemistry, 1969, 7(4), 547-54 and its deprotonation with NaH/DMF or NaH/DMF and its subsequent displacement of M, wherein M is a suitable leaving group such as chloride, bromide or iodide, yields I-36′.

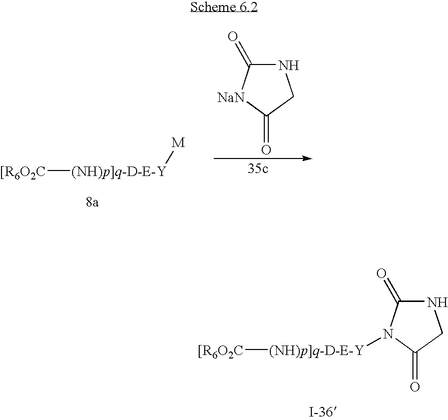

Compounds of Formula I wherein Q is Q-10 or Q-11, and Y is alkylene are prepared as shown in Schemes 7.1 and 7.2, respectively. Treatment of alcohol 37 (Z=O) or amine 37 (Z=NH) with chlorosulfonylisocyanate affords intermediate carbamate or urea of structure 38. Treatment of 38 with an amine of structure HN(R₄)₂ and base, preferably triethylamine or pyridine, gives sulfonylureas of Formula I-39. Reaction of chlorosulonylisocyanate with (an alcohol (Z=O) or amine (Z=NR₄) 40 affords intermediate 41. Treatment of 41 with an amine 8 and base, preferably triethylamine or pyridine, gives sulfonylureas of Formula I-42.

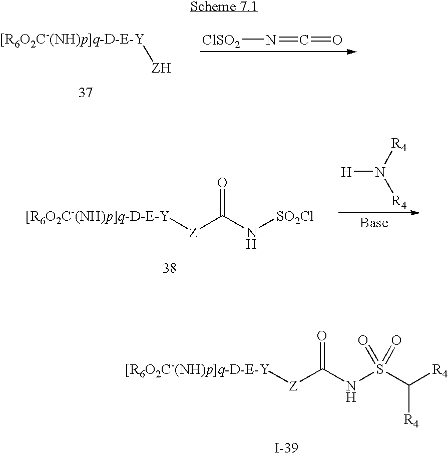

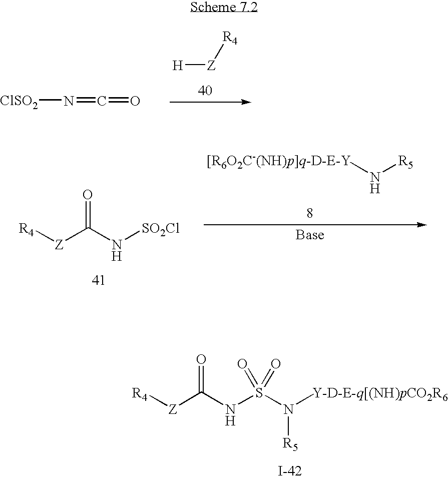

Compounds of Formula I wherein Q is taken from Q-12 are prepared according to the synthetic route shown in Scheme 8. Alkylation of pyridine 43, wherein TIPS is tri-isopropylsilyl, under standard conditions (K₂CO₃, DMF, R₄—I or Mitsunobu conditions employing R₄—OH) yields pyridine derivative 44 which is reacted with compound 12, wherein M is a suitable leaving group, to afford pyridones of formula I-45.

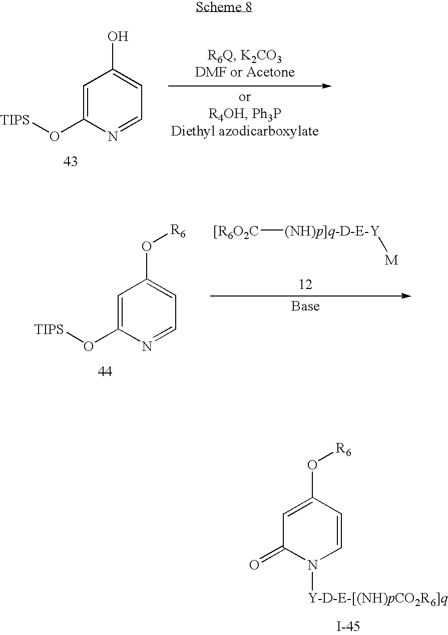

Compounds of Formula I wherein Q is taken from Q-13 are prepared according to the synthetic route shown in Scheme 9. Starting from readily available pyridine 46, alkylation under standard conditions (K₂CO₃, DMF, R₄—I or Mitsunobu conditions employing R₄—OH) yields pyridine derivative 47. N-alkylation with K₂CO₃, DMF. R₄—I affords pyridones of formula 48. Intermediate 48 is partitioned to undergo a Heck reaction, giving I-49; a Buchwald amination reaction, giving I-51; or a Buchwald Cu(I) catalyzed O-arylation reaction, to give I-52. The Heck reaction product I-49 may be optionally hydrogenated to afford the saturated compound I-50. Wherein the phenyl ether R₄ group is methyl, compounds of formula I-49, I-50, I-51, or I-52 are treated with boron tribromide or lithium chloride to afford compounds of Formula I-53, wherein R₄ is hydrogen.

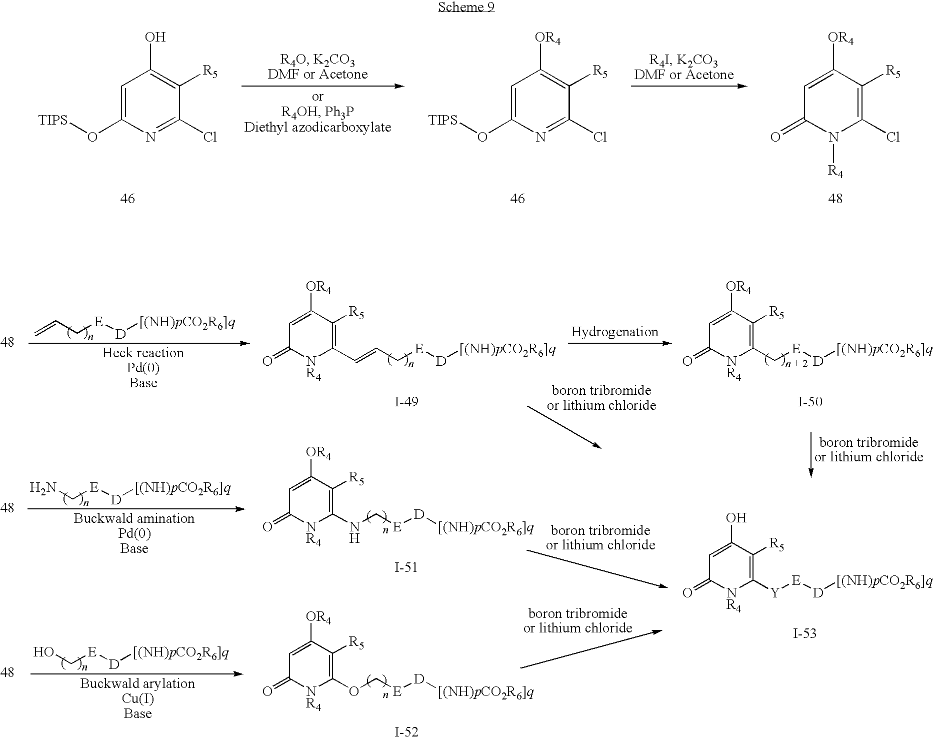

Compounds of Formula I wherein Q is taken from Q-14 are prepared according to the synthetic route shown in Scheme 10. Starting from readily available pyridine 54, alkylation under standard conditions (K₂CO₃, DMF, R₄—I or Mitsunobu conditions employing R₄—OH) yields pyridine derivative 55. N-alkylation with K₂CO₃, DMF, R₄—I affords pyridones of formula 56. Intermediate 56, wherein M is a suitable leaving group, preferably bromine or chlorine, is partitioned to undergo a Heck reaction, giving I-57; a Buchwald amination reaction, giving I-59; or a Buchwald Cu(I) catalyzed O-arylation reaction, to give I-60. The Heck reaction product I-57 may be optionally hydrogenated to afford the saturated compound I-58. Wherein R₄ is methyl, compounds of formula I-57, I-58, I-59, or I-60 are treated with boron tribromide or lithium chloride to afford compounds of Formula I-61, wherein R₄ is hydrogen.

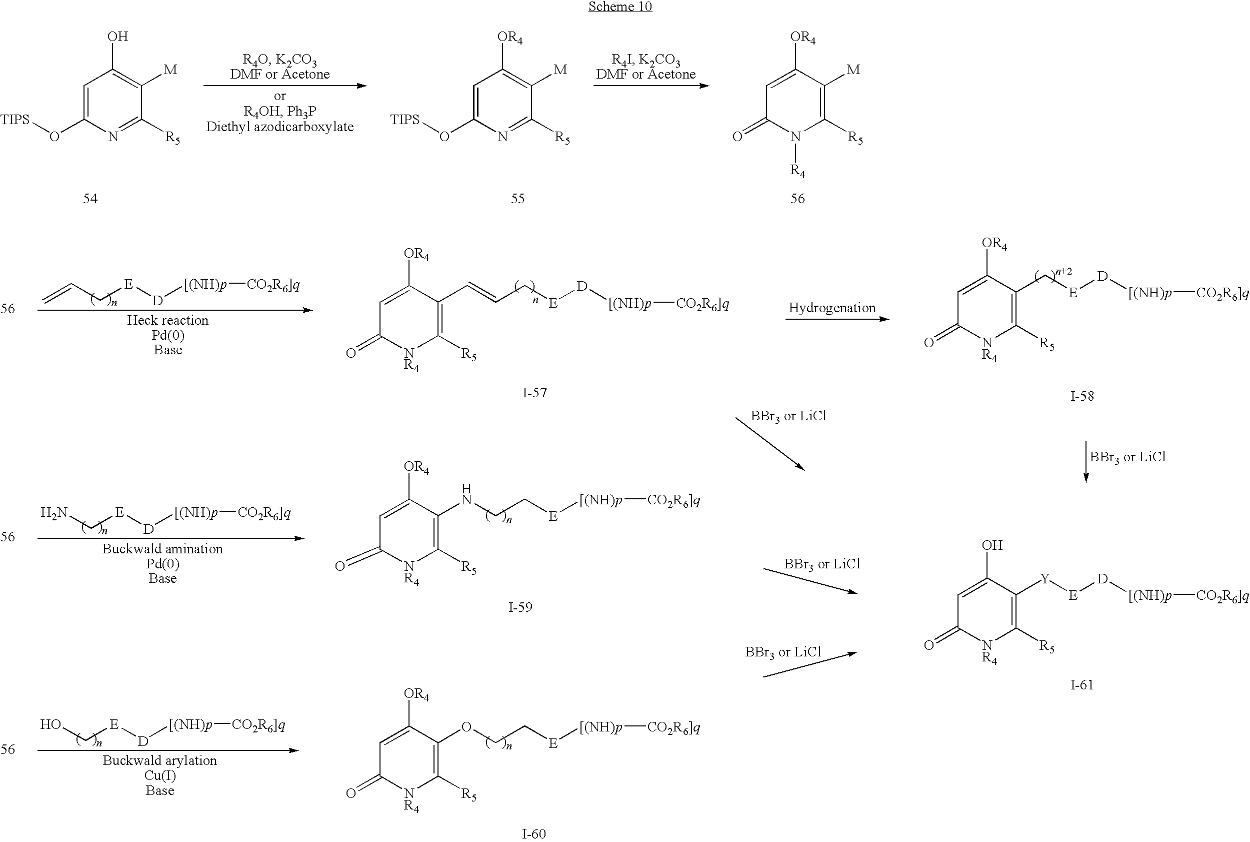

Compounds of Formula I wherein Q is taken from Q-15 are prepared according to the synthetic routes shown in Schemes 11 and 12. Starting esters 62 are available from the corresponding secoacids via TBS-ether and ester formation under standard conditions. Reaction of protected secoester 62 with Meerwin's salt produces the vinyl ether 63 as a pair of regioisomers. Alternatively, reaction of 62 with dimethylamine affords the vinylogous carbamate 64. Formation of the dihydropyrimidinedione 66 proceeds by condensation with urea 65 with azeotropic removal of dimethylamine or methanol. Dihydropyrimidinedione 66 may optionally be further substituted by Mitsunobu reaction with alcohols R₄OH to give rise to compounds 67.

Scheme 12 illustrates the further synthetic elaboration of intermediates 67. Removal of the silyl protecting group (TBS) is accomplished by treatment of 67 with flouride (tetra-n-butylammonium fluoride or cesium flouride) to give primary alcohols 68. Reaction of 68 with isocyanates 2 gives rise to compounds of Formula I-69. Alternatively, reaction of 68 with [R₆O₂C(NH)ₚ]_{q}-D-E-M, wherein M is a suitable leaving group, affords compounds of Formula I-70. Oxidation of 68 using the Dess-Martin periodinane (D. Dess, J. Martin, J. Am. Chem. Soc. (1991) 113:7277) or tetra-n-alkyl peruthenate (W. Griffith, S. Ley, Aldrichimica Acta (1990) 23:13) gives the aldehydes 71. Reductive amination of 71 with amines 8 gives rise to compounds of Formula I-72. Alternatively, aldehydes 71 may be reacted with ammonium acetate under reductive alkylation conditions to give rise to the primary amine 73. Reaction of 73 with isocyanates 2 affords compounds of Formula I-74.

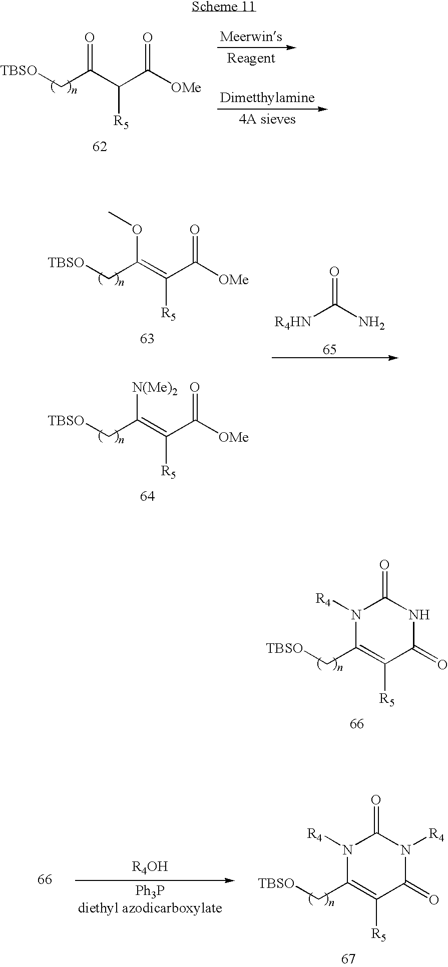

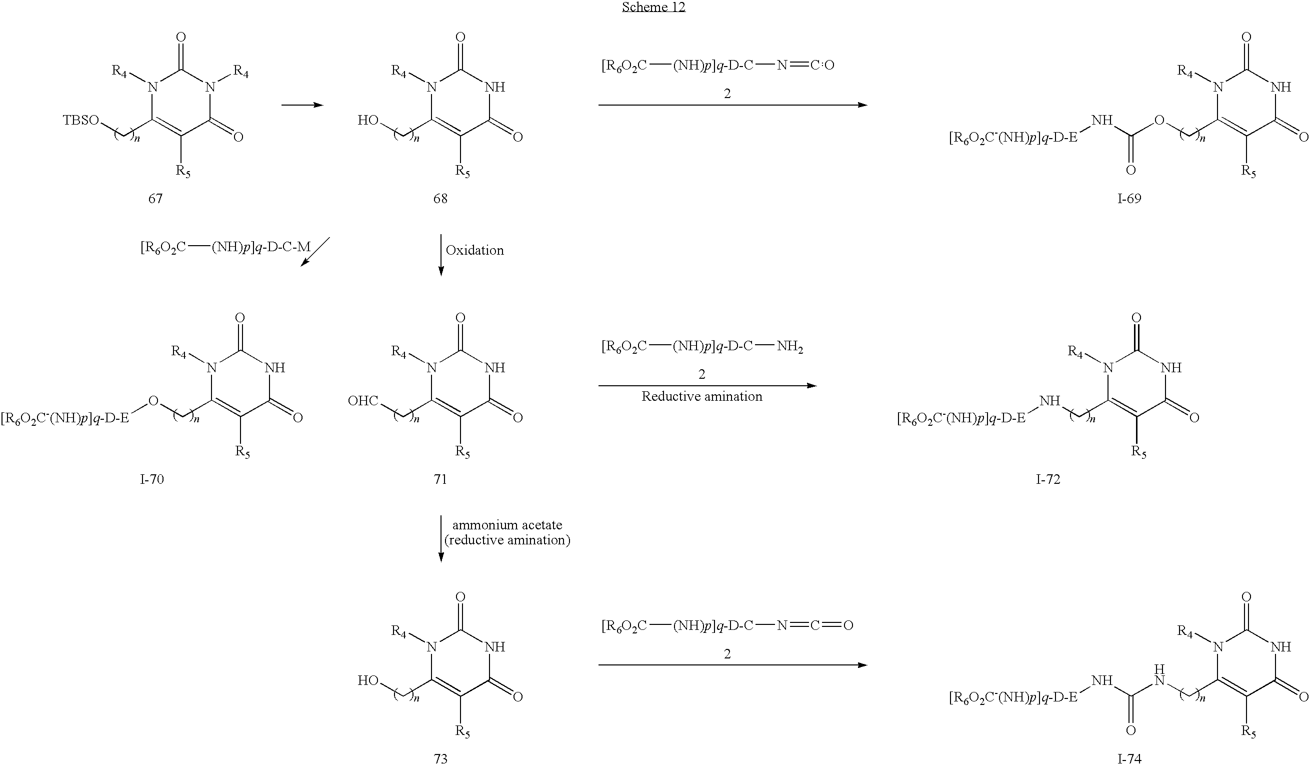

Compounds of Formula I wherein Q is taken from Q-16 are prepared according to the synthetic routes shown in Schemes 13 and 14. Starting esters 75 are available from the corresponding secoacids via TBS-ether and ester formation under standard conditions. Reaction of protected secoester 75 with Meerwin's salt produces the vinyl ether 76 as a pair of regioisomers. Alternatively, reaction of 75 with dimethylanaine affords the vinylogous carbamate 77. Formation of the dihydropyrimidinedione 78 proceeds by condensation with urea 65 with azeotropic removal of dimethylamine or methanol. Dihydropyrimidinedione 78 may optionally be further substituted by Mitsunobu reaction with alcohols R₄OH to give rise to compounds 79. Compounds of Formulae I-81, I-82, I-84, and I-86 are prepared as shown in Scheme 14 by analogy to the sequence previously described in Scheme 12.

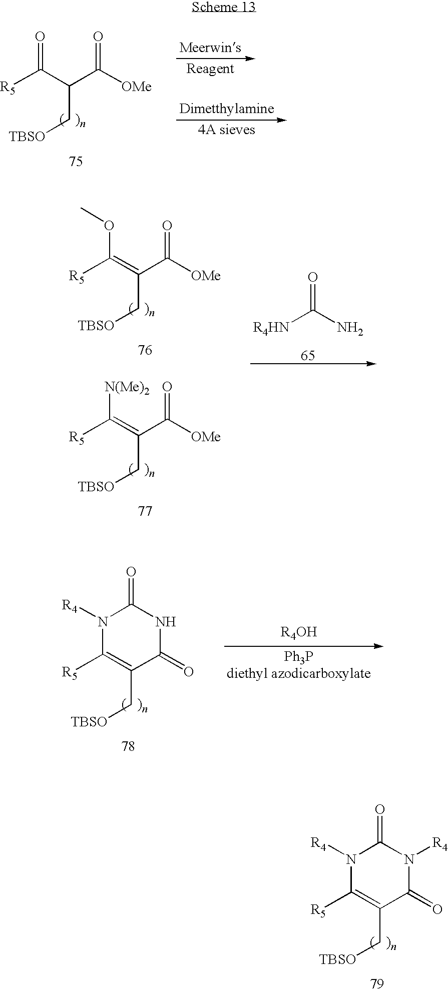

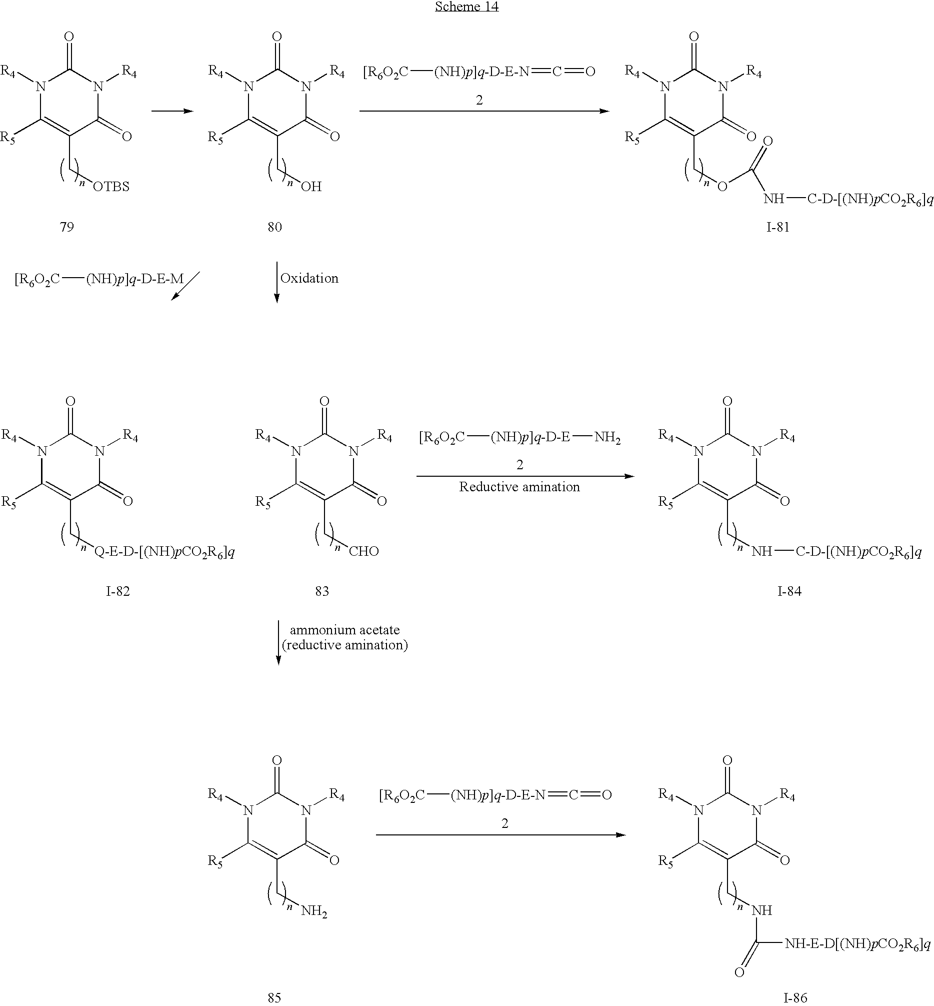

Alkyl acetoacetates 87 are commercially available and are directly converted into the esters 88 as shown in Scheme 15. Treatment of 87 with NaHMDS in THF, followed by quench with formaldehyde and TBSCl (n=1) or Q-(CH₂)ₙ-OTBS (n=24), gives rise to compounds 88.

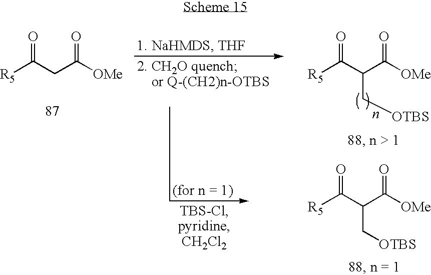

Compounds of Formula I wherein Q is taken from Q-17 are prepared according to the synthetic routes shown in Schemes 16.1 and 16.2, and starts with the BOC-protected hydrazine 13, which is converted to the 1,2-disubstituted hydrazine 89 by a reductive alkylation with a glyoxal derivative mediated by sodium cyanoborohydride and acidic workup. Condensation of 89 with diethyl malonate in benzene under reflux yields the heterocycle 90. Oxidation with N₂O₄ in benzene (see Cardillo, Merlini and Boeri Gazz. Chim. Ital., (1966) 9:8) to the nitromalonohydrazide 91 and further treatment with P₂O₅ in benzene (see: Cardillo, G. et al, Gazz. Chim. Ital. (1966) 9:973-985) yields the tricarbonyl 92. Alternatively, treatment of 90 with Brederick's reagent (t-BuOCH(N(Me₂)₂, gives rise to 93, which is subjected to ozonolysis, with a DMS and methanol workup, to afford the protected tricarbonyl 92. Compound 92 is readily deprotected by the action of CsF in THF to yield the primary alcohol 94. Alcohol 94 is optionally converted into the primary amine 95 by a sequence involving tosylate formation, azide displacement, and hydrogenation.

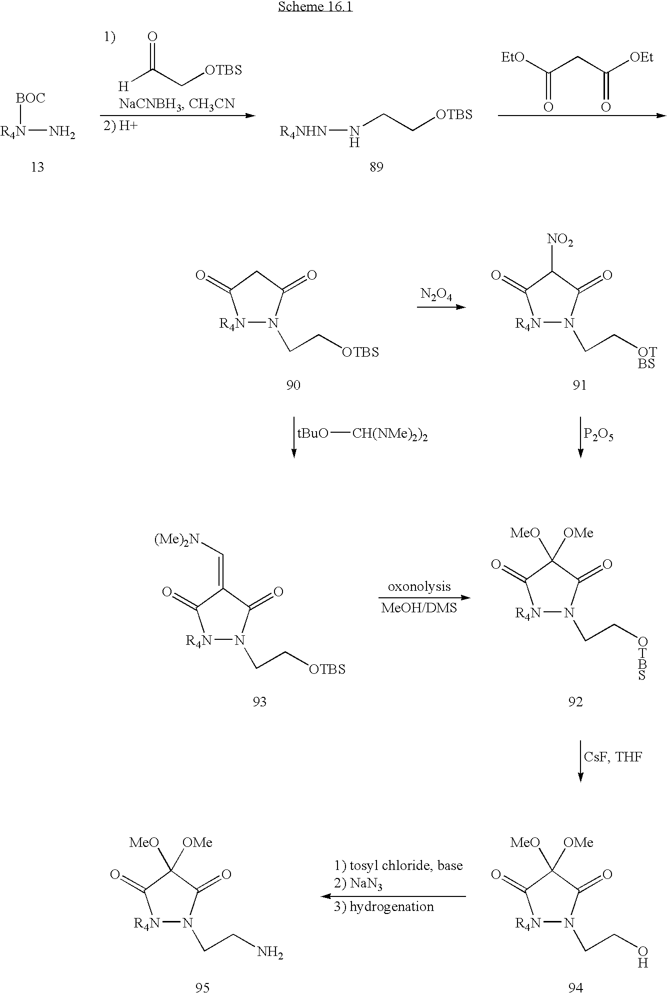

Reaction of 94 with (hetero)aryl halide 26, wherein M is iodo, bromo, or chloro, under copper(I) catalysis affords compounds I-96. Optional deprotection of the di-methyl ketal with aqueous acid gives rise to compounds of Formula I-98. By analogy, reaction of amine 95 with 26 under palladium(0) catalysis affords compounds of Formula I-97. Optional deprotection of the di-methyl ketal with aqueous acid gives rise to compounds of Formula I-99.

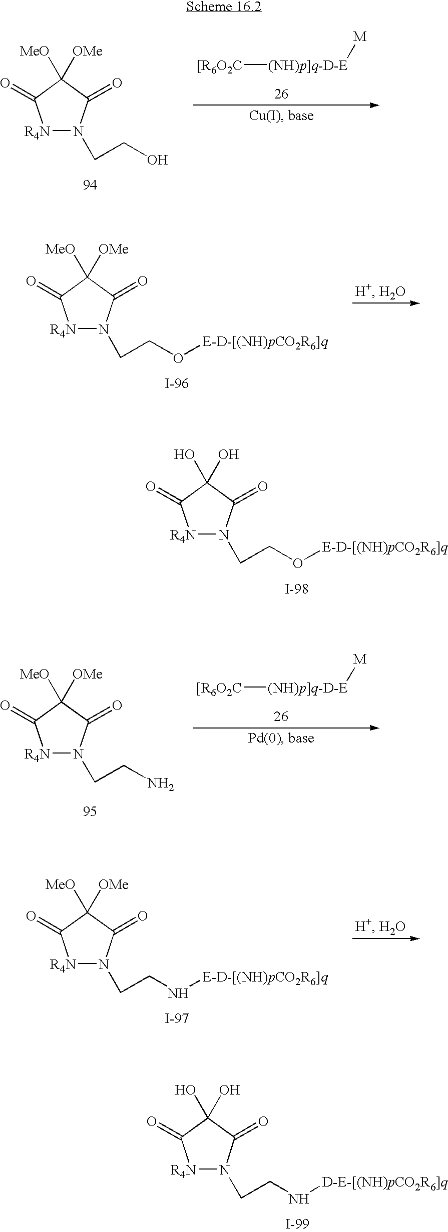

Compounds of Formula I wherein Q is taken from Q-17 are also prepared according to the synthetic route shown in Scheme 16.3. Deprotonation of 4,4-dimethyl-3,5-dioxo-pyrazolidine (95a, prepared according to the method described in Zinner and Boese, D. Pharmazie 1970, 25(5-6), 309-12 and Bausch, M. J. et. al J. Org. Chem. 1991, 56(19), 5643) with NaH/DMF or NaH/DMF and its subsequent displacement of M, wherein M is a suitable leaving group such as chloride, bromide or iodide yields I-99a.

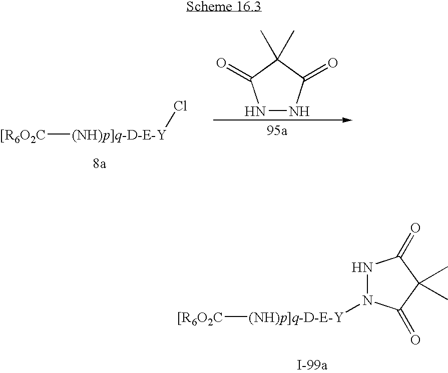

Compounds of Formula I wherein Q is taken from Q-18 are prepared as shown in Schemes 17.1 and 17.2. Aminoesters 100 are subjected to reductive alkylation conditions to give rise to intermediates 101. Condensation of amines 101 with carboxylic acids using an acid activating reagent such as dicyclohexylcarbodiimide (DCC)/hydroxybenzotriazole (HOBt) affords intermediate amides 102. Cyclization of amides 102 to tetramic acids 104 is mediated by Amberlyst A-26 hydroxide resin after trapping of the in situ generated alkoxide 103 and submitting 103 to an acetic acid-mediated resin-release.

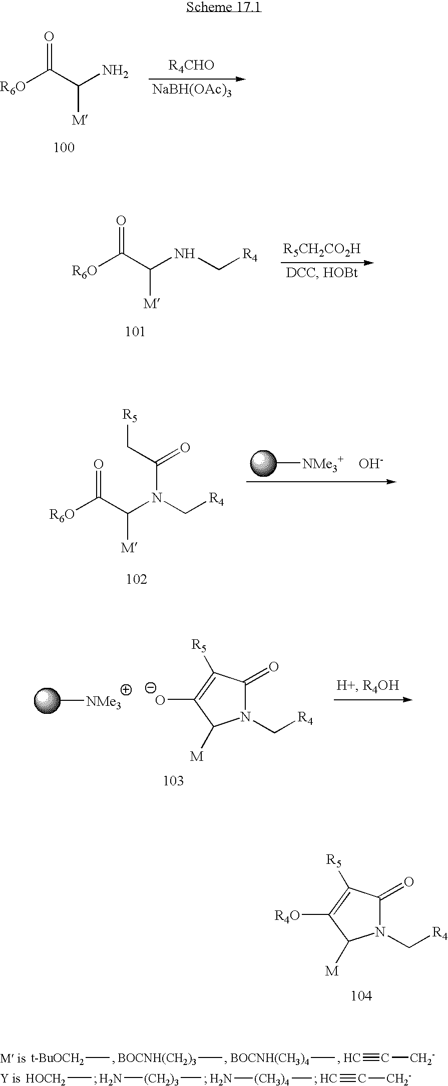

Scheme 17.2 illustrates the synthetic sequences for converting intermediates 104 to compounds of Formula I. Reaction of alcohol 104.1 with aryl or heteroaryl halide 26 (Q=halogen) under copper(I) catalysis gives rise to compounds of Formula I-105.1. Reaction of amines 104.2 and 104.3 with 26 under Buchwald palladiuim(0) catalyzed amination conditions affords compounds of Formulae I-105.2 and I-105.3. Reaction of acetylene 104.4 with 26 under Sonogashira coupling conditions affords compounds of Formula I-105.4. Compounds I-105.4 may optionally be reduced to the corresponding saturated analogs I-105.5 by standard hydrogenation.

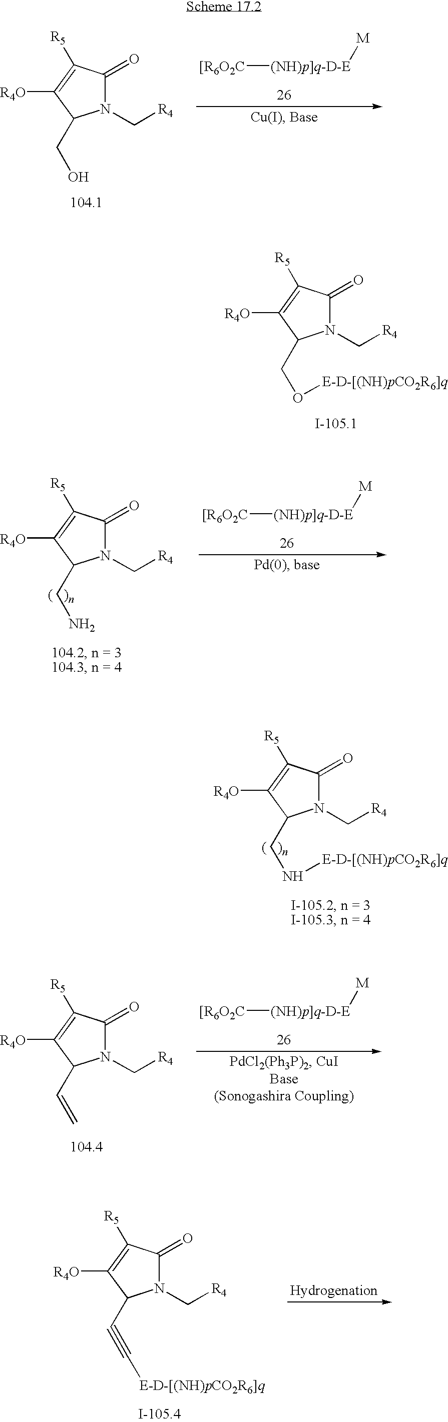

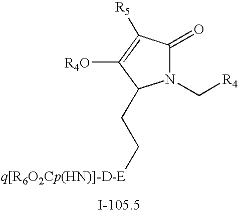

Compounds of Formula I wherein Q is taken from Q-19, Q-20, or Q-21 are prepared as illustrated in Scheme 18. Commercially available Kemp's acid 106 is converted to its anhydride 107 using a dehydrating reagent, preferably di-isopropylcarbodiimide (DIC) or 1-(3-dimethylaminopropyl)-3-ethylcarbodiimide (EDC). Reaction of 107 with amines R₄NH₂ affords the intermediate amides which are cyclized to the imides 108 by reaction with DIC or EDC. Alternatively, 107 is reacted with amines 8 to afford amides of Formula I-110. Amides I-110 may optionally be further reacted with DIC or EDC to give rise to compounds of Formula I-111. Acid 108 is further reacted with amines 8 to give compounds of Formula I-109.

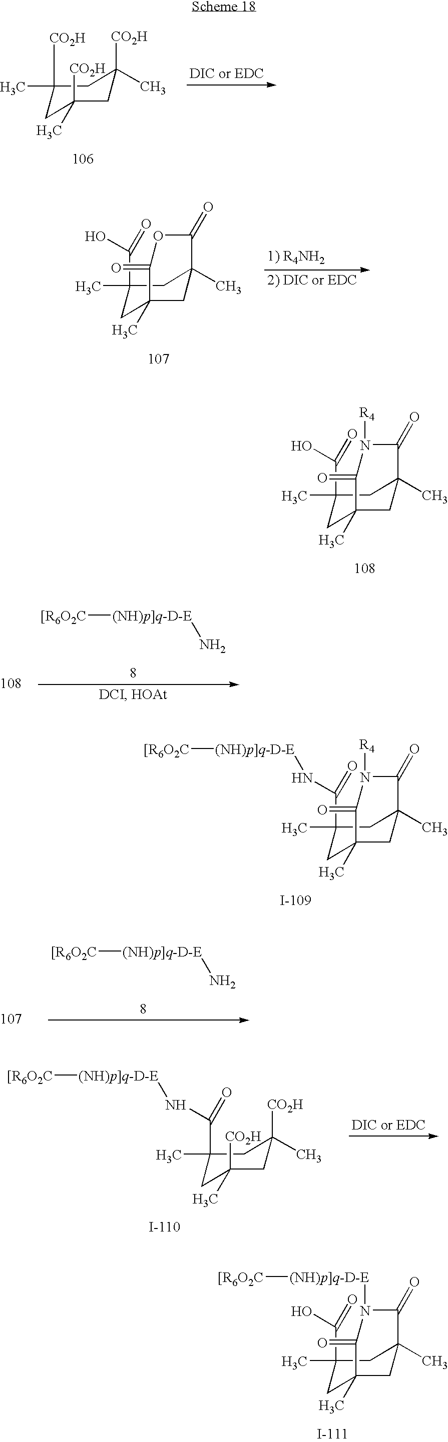

Compounds of Formula I wherein Q is taken from Q-22 or Q-23 are prepared as shown in Schemes 19.1 through 19.3. Preparation of intermediates 113 and 114 are prepared as shown in Scheme 19.1 from di-halo(hetero)aryls 112, wherein M₂ is a more robust leaving group than M₁. Reaction of 112 with amines 37 (Z=NH) either thermally in the presence of base or by palladium(0) catalysis in the presence of base and phosphine ligand affords compounds 113. Alternatively, reaction of 112 with alcohols 37 (X═O) either thermally in the presence of base or by copper(I) catalysis in the presence of base affords compounds 114.

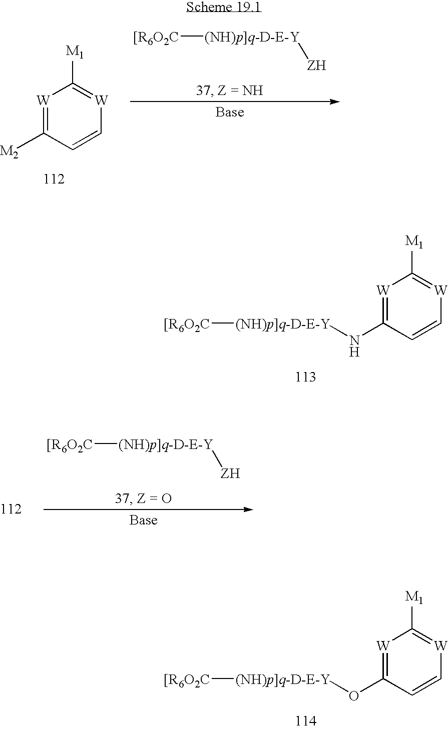

Scheme 19.2 illustrates the conversion of intermediates 113 into compounds of Formula I-115, I-118, or 117. Treatment of 113 with aqueous copper oxide or an alkaline hydroxide affords compounds of Formula I-115. Alternatively, treatment of 113 with t-butylmercaptan under copper(I) catalysis in the presence of ethylene glycol and potassium carbonate gives rise to 116 (see F. Y. Kwong and S. L. Buchwald, Organic Letters (2002) 4:3517. Treatment of the t-butyl sulfide 116 with acid affords the desired thiols of Formula I-118. Alternatively, 113 may be treated with excess ammonia under pressurized conditions to afford compound 117.

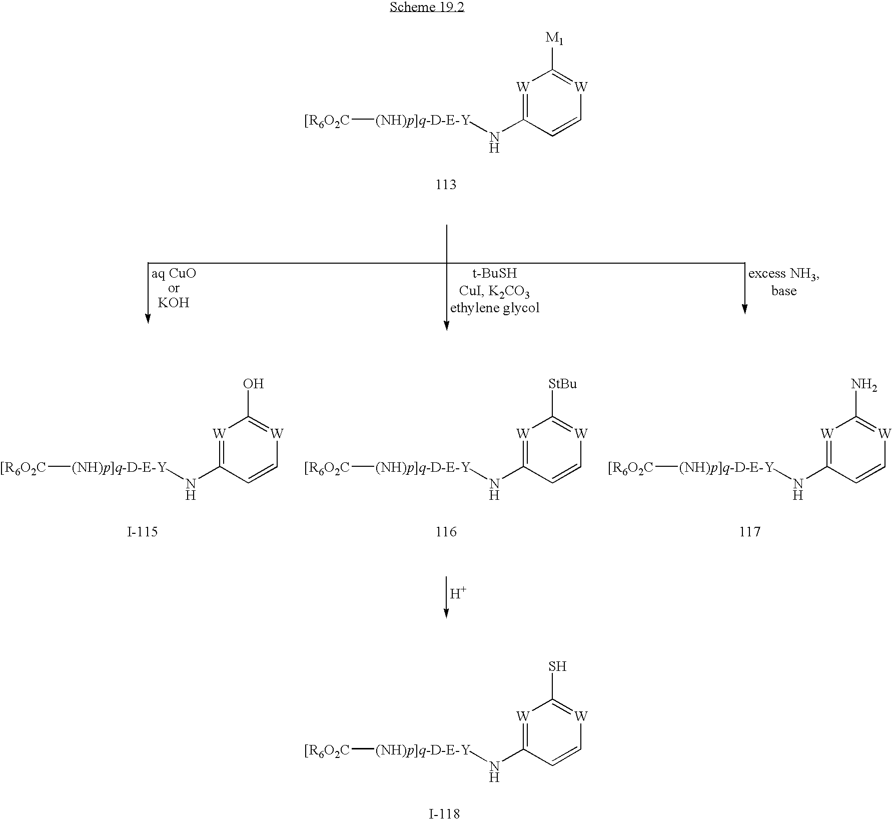

Scheme 19.3 illustrates the conversion of intermediate 114 into compounds of Formula I-19, I-122, and 121, by analogy to the sequence described in Scheme 19.2.

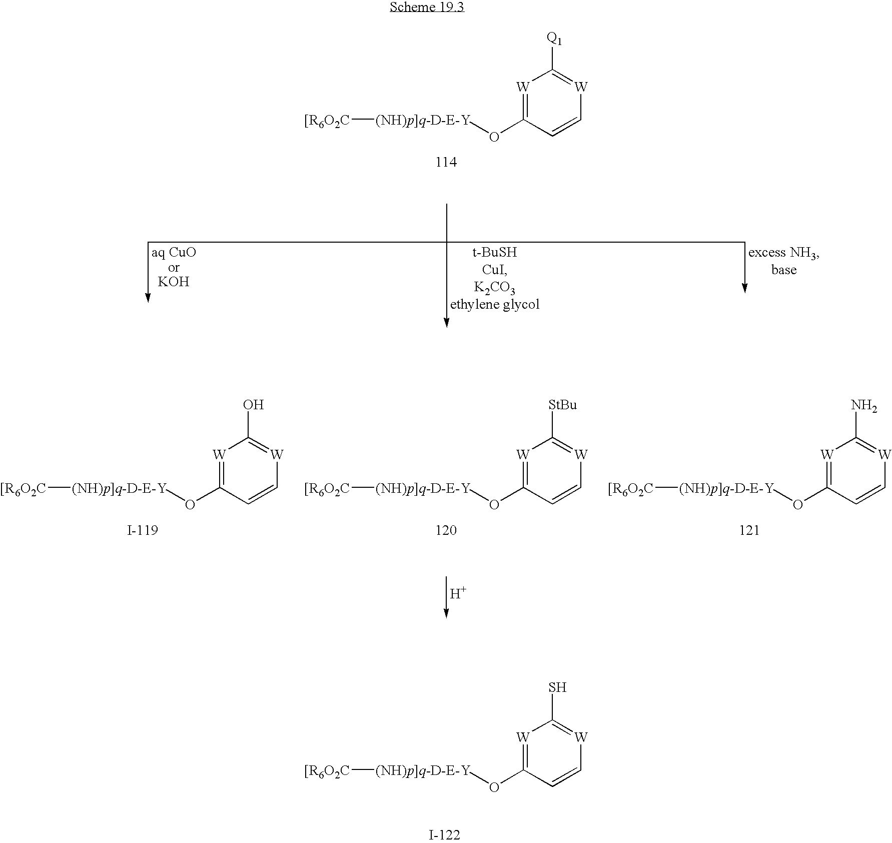

Compounds of Formula I wherein q is taken from Q-24, Q-25, or Q-26 are prepared as shown in Scheme 20. Reaction of compounds I-115 or I-119 with chlorosulfonylisocyanate, followed by in situ reaction with amines HN(R₄)₂ gives rise to compounds of Formulae I-123 or I-124. Reaction of compounds I-118 or I-122 with a peracid, preferably peracetic acid or trifluoroperacetic acid, affords compounds of Formula I-125 or I-126. Reaction of compounds 117 or 121 with chlorosulfonylisocyanate, followed by in situ reaction with amines HN(R₄) or alcohols R₄OH, affords compounds of Formulae I-127, I-128, I-129, or I-130.

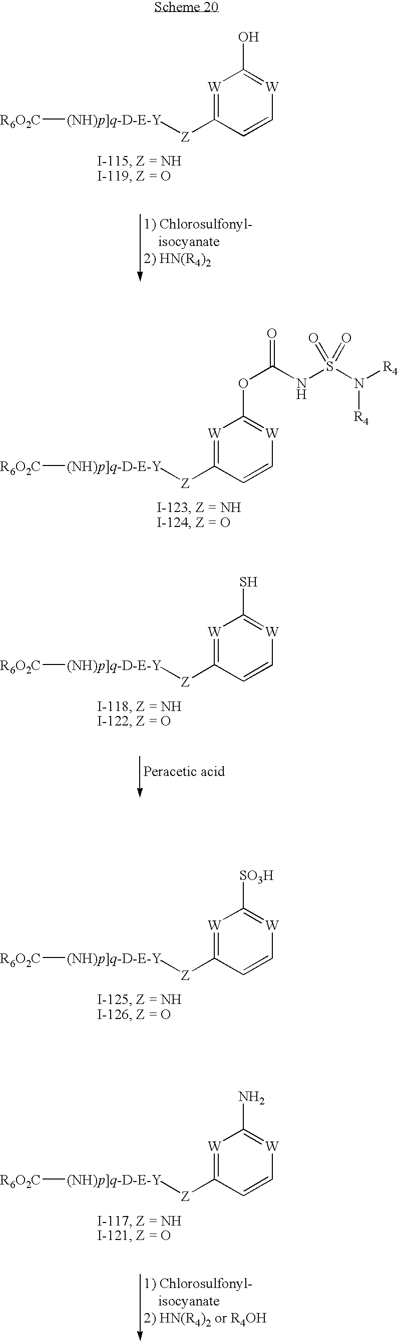

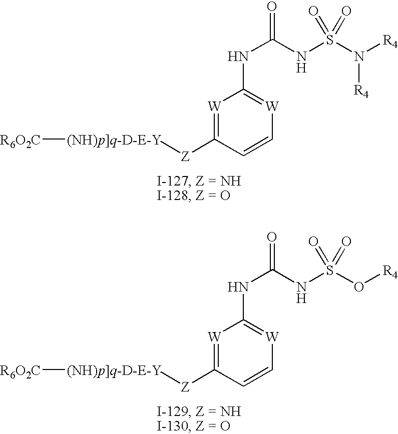

Compounds of Formula I wherein Q is taken from Q-27 are prepared as illustrated in Scheme 21. Reductive alkylation of thiomorpholine with aldehydes 131 affords benzylic amines 132, which are then subjected to peracid oxidation to give rise to the thiomorpholine sulfones 133 (see C. R. Johnson et al, Tetrahedron (1969) 25: 5649). Intermediates 133 are reacted with amines 8 (Z=NH₂) under Buchwald palladium-catalyzed amination conditions to give rise to compounds of Formula I-134. Alternatively, compounds 133 are reacted with alcohols 8 (Z=OH) under Buchwald copper(I) catalyzed conditions to afford compounds of Formula I-135. Alternatively, intermediates 133 are reacted with alkenes under palladium(0)-catalyzed Heck reaction conditions to give compounds of Formula I-136. Compounds I-136 are optionally reduced to the corresponding saturated analogs I-137 by standard hydrogenation conditions or by the action of diimide.

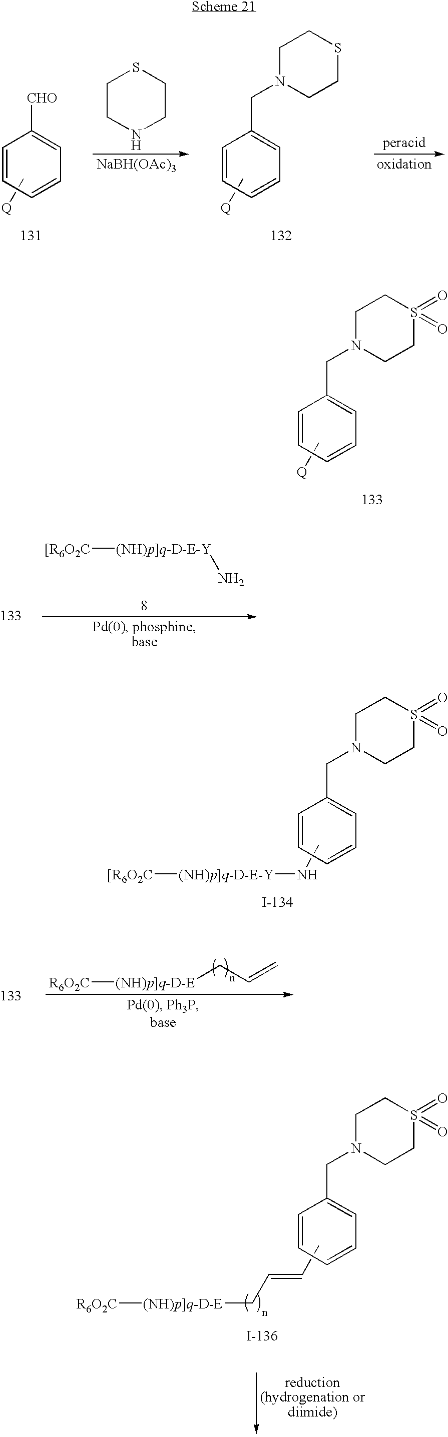

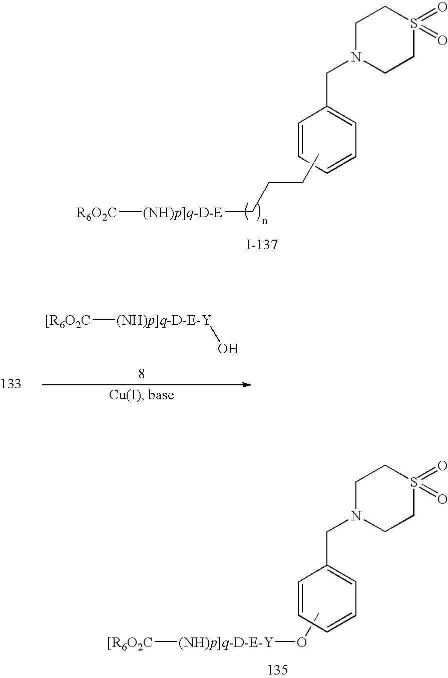

Compounds of Formula I wherein Q is taken from Q-27 are also prepared as illustrated in Scheme 21.1. Aldehyde 8c is reductively aminated with ammonia, and the resultant amine condensed with divinyl sulphone to yield I-134. Intermediate 134a is also available by reduction of amide 8d under a variety of standard conditions.

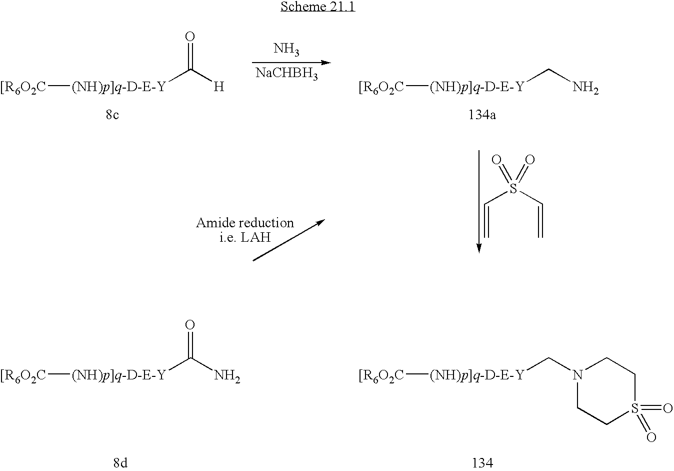

More generally, compounds of formula I wherein Q is taken from Q43 and represent amines 134c are available via the reduction of amides 134b as shown in Scheme 21.2. The morpholine amide analogues 134d and morpholine analogues 134e are also available as shown in Scheme 21.2.

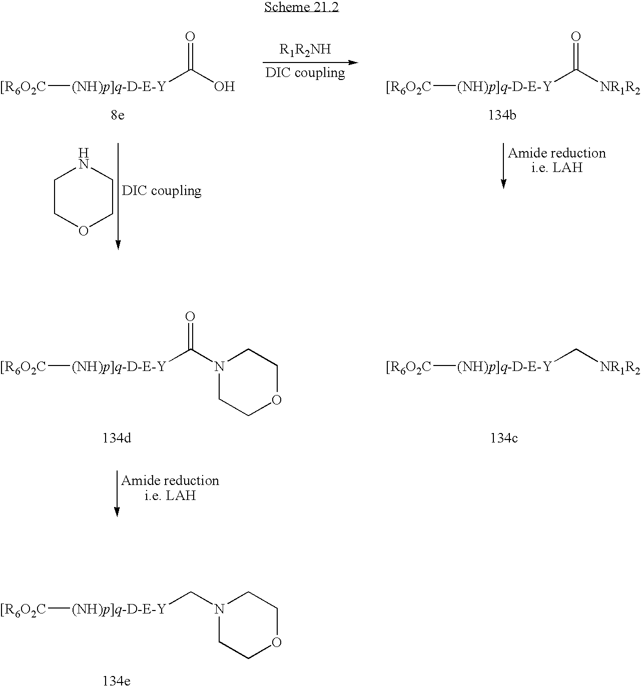

Compounds of Formula I wherein Q is taken from Q-28 or Q-29 are prepared according to the sequences illustrated in Scheme 22. Readily available amides 138 are reacted with chlorosulfonylisocyanate to give intermediates 140 which are reacted in situ with amines HN(R₄)₂ or alcohols ROH to afford compounds of Formulae I-141 or I-142, respectively. Alternatively, amides 138 are reacted with sulfonylchlorides to give compounds of Formula I-139.

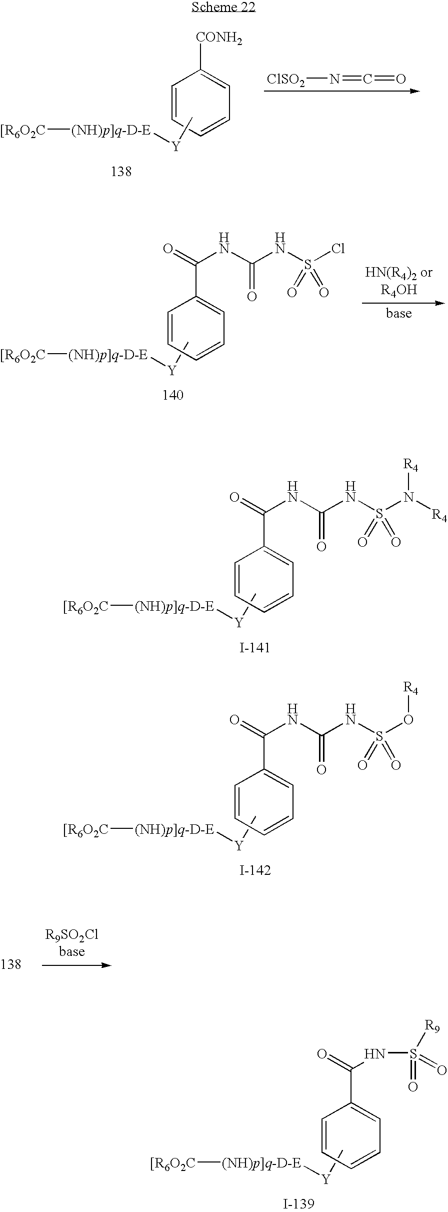

Compounds of Formula I wherein Q is taken from Q-30 are prepared as shown in Scheme 23. Readily available N—BOC anhydride 143 (see S. Chen et al, J. Am. Chem. Soc. (1996) 118:2567) is reacted with amines HN(R₄)₂ or alcohols R₆OH to afford acids 144 or 145, respectively. Intermediates 144 or 145 are further reacted with amines HN(R₄)₂ in the presence of an acid-activating reagent, preferably PyBOP and di-isopropylethylamine, to give diamides 146 or ester-amides 147. Intermediate 145 is converted to the diesters 148 by reaction with an alkyl iodide in the presence of base, preferably potassium carbonate. Intermediates 146-148 are treated with HCl/dioxane to give the secondary amines 149-151, which are then condensed with acids 152 in the presence of PyBOP and di-isopropylethylamine to give compounds of Formula I-153.

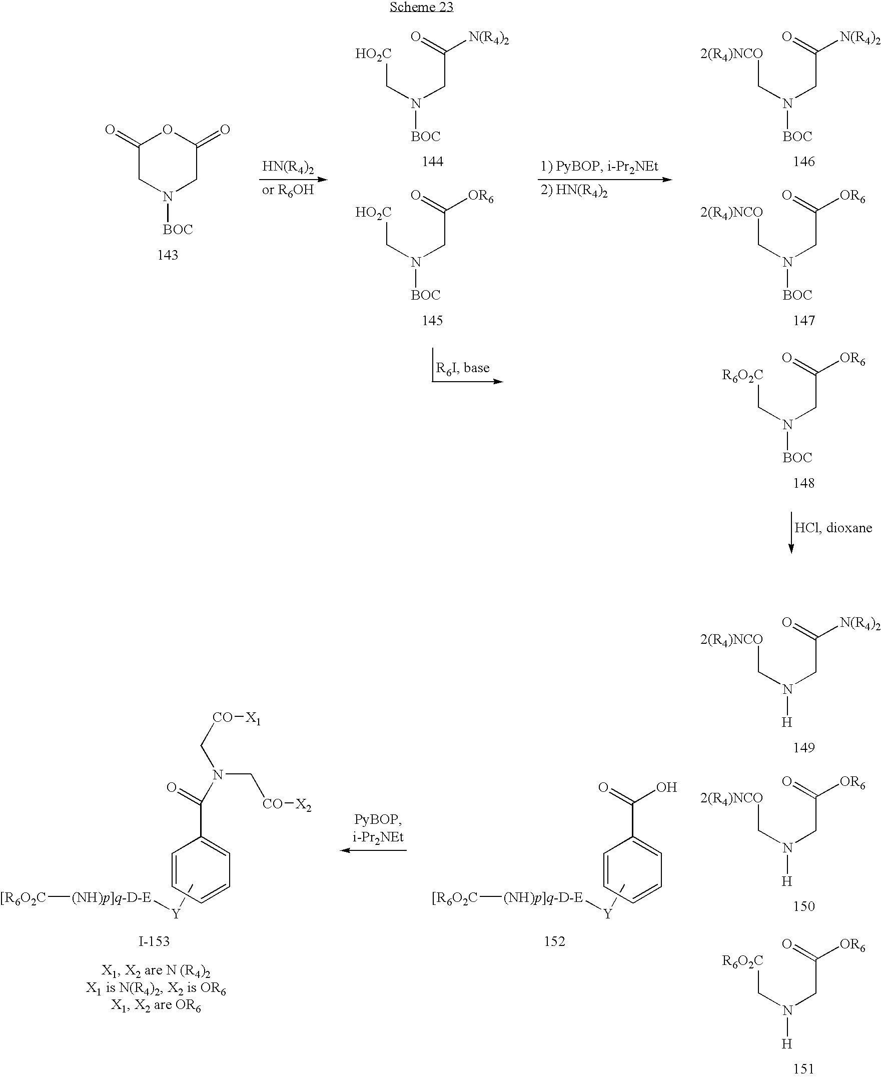

Compounds of Formula I wherein Q is taken from Q-31 or Q-32 are prepared according to the sequences illustrated in Scheme 24. Treatment of readily available sulfenamides 154 with amines 37 (Z=NH), alcohols 37 (Z=O), or alkenes 37 (Z=-CH═CH₂), gives rise to compounds of Formula I-155. Treatment of sulfenamides I-155 with iodosobenzene in the presence of alcohols R₆OH gives rise to the sulfonimidates of Formula I-157 (see D. Leca et al, Organic Letters (2002) 4:4093). Alternatively, compounds I-155 (Z=—CH═CH) may be optionally reduced to the saturated analogs I-156 (Z=CH₂—CH₂—), which are converted to the corresponding sulfonimidates I-157.

Treatment of readily available sulfonylchlorides 154.1 with amines HN(R₄)₂ and base gives rise to compounds of Formula I-154.2.

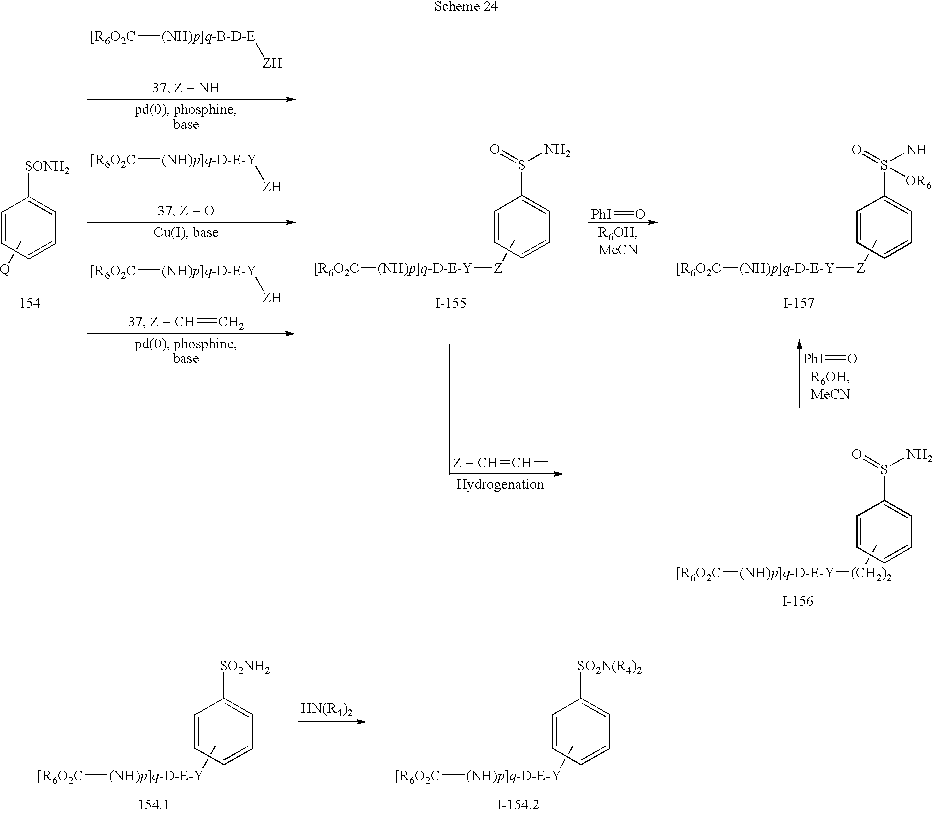

Compounds of Formula I wherein Q is taken from Q-33 or Q-48A are prepared as shown in Scheme 25. Readily available nitrites 158 are reacted with amines 37 (Z=NH), alcohols 37 (Z=O), or alkenes 37 (Z=-CH═CH₂) to afford compounds of Formula I-159. Compounds I-159 (wherein Z=CH═CH—) are optionally reduced to their saturated analogs I-160 by standard catalytic hydrogenation conditions. Treatment of compounds I-159 or I-160 with a metal azide (preferably sodium azide or zinc azide) gives rise to tetrazoles of Formula I-161.

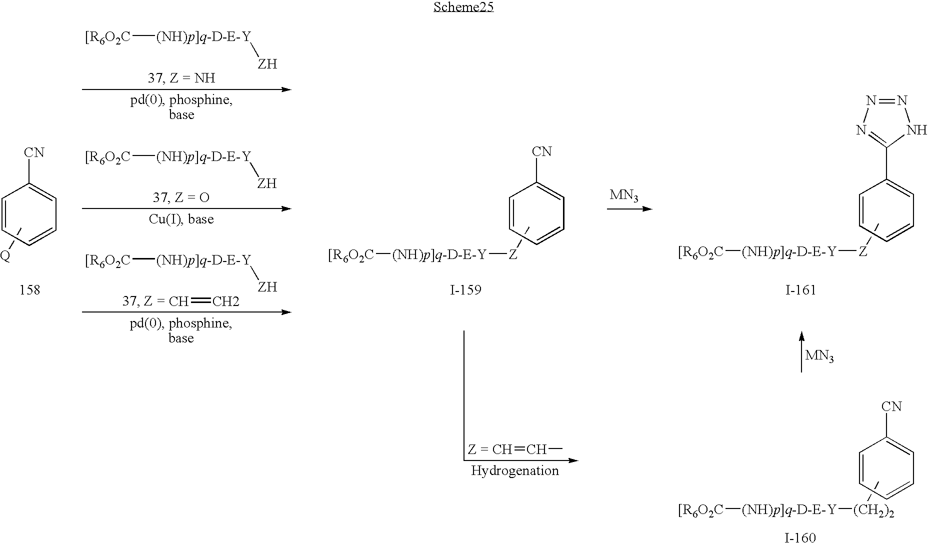

Compounds of Formula I wherein Q is taken from Q-34 are prepared as shown in Scheme 26. Readily available esters 162 are reacted with amines 37 (Z=NH), alcohols 37 (Z=O), or alkenes 37 (Z=-CH═CH₂) to afford compounds of Formula I-163. Compounds I-163 (wherein Z is —CH═CH—) are optionally converted to the saturated analogs I-164 by standard hydrogenation conditions. Compounds I-163 or I-164 are converted to the desired phosphonates I-165 by an Arbuzov reaction sequence involving reduction of the esters to benzylic alcohols, conversion of the alcohols to the benzylic bromides, and treatment of the bromides with a tri-alkylphosphite. Optionally, phosphonates I-165 are converted to the flourinated analogs I-166 by treatment with diethylaminosulfur trifluoride (DAST).

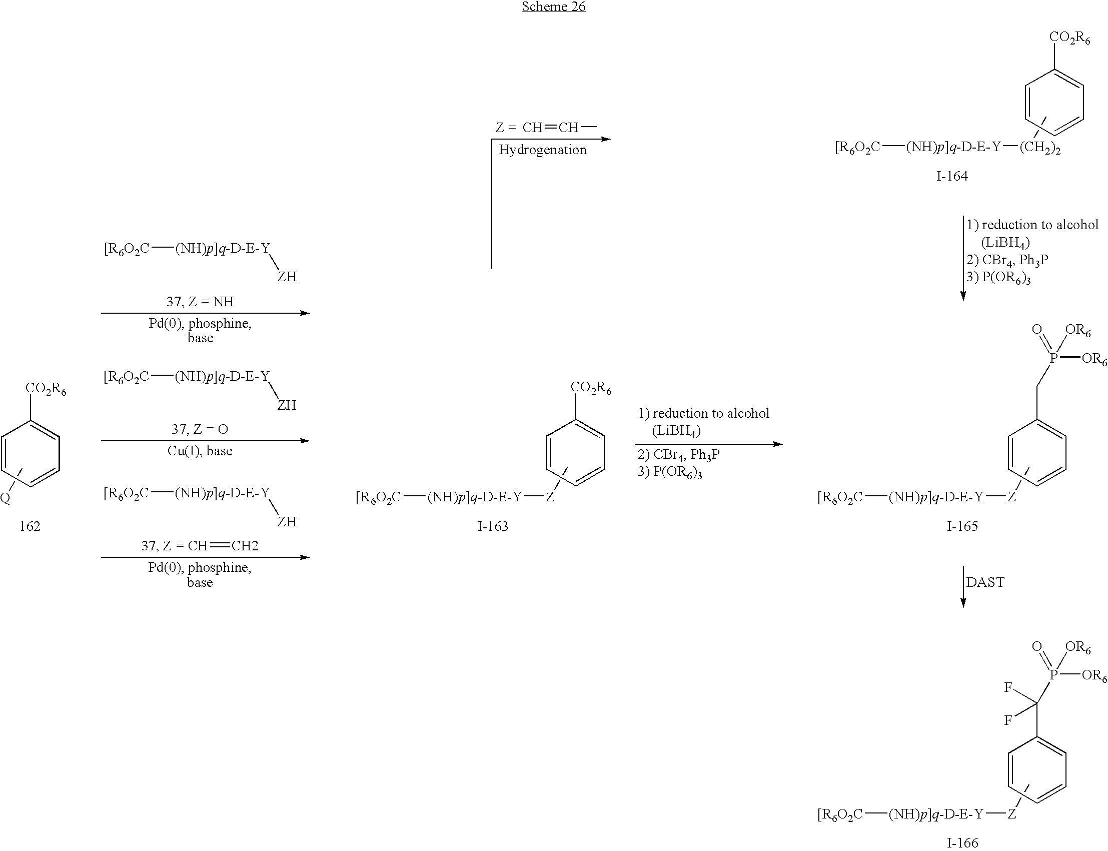

Compounds of Formula I wherein Q is taken from Q-34 are also prepared as illustrated in Scheme 27.1. Intermediate 8a, wherein M is a suitable leaving group such as chloride, bromide or iodide, is refluxed with triethyl phosphite and the resulting phosphoryl intermediate saponified under mild conditions to yield I-165.

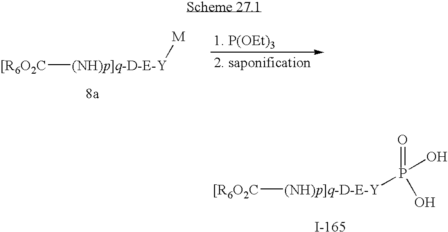

Compounds of Formula I wherein Q is taken from Q-35 are prepared according to Scheme 27. Readily available acid chlorides 167 are reacted with oxazolidones in the presence of base to afford the N-acyl oxazolidinones 168. Intermediate 168 are reacted with amines 37 (Z=NH), alcohols 37 (Z=O), or alkenes 37 (Z=-CH═CH₂) to afford the N-acyl oxazolidinones of Formula I-169. Compounds I-169 (wherein Z is —CH═CH—) are optionally converted to the saturated analogs I-170 under standard hydrogenation conditions.

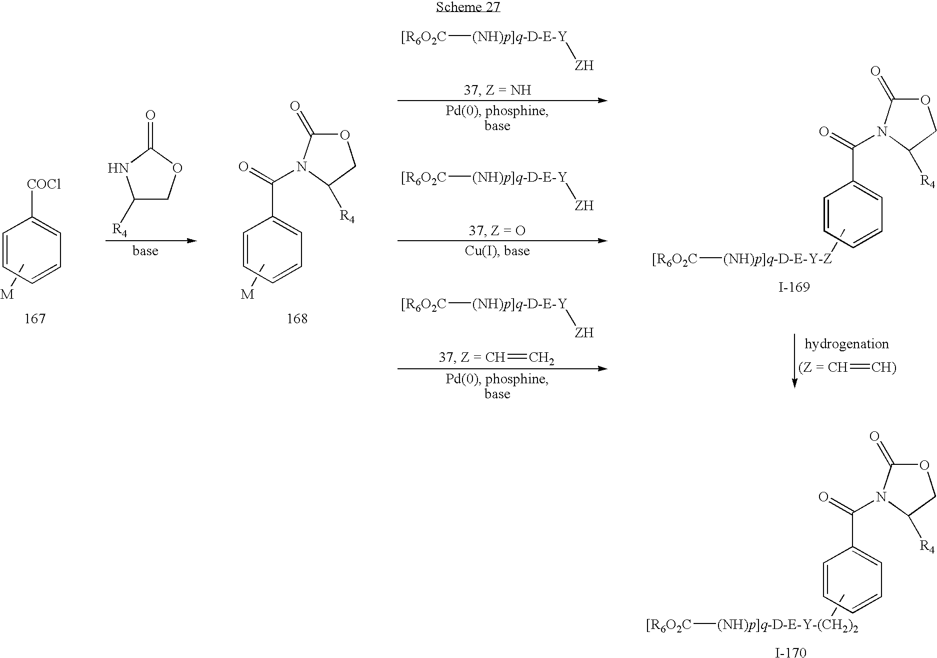

Compounds of Formula I wherein Q is taken from Q-35A are prepared as illustrated in Schemes 28.1 and 28.2. Reductive alkylation of the t-butylsulfide substituted piperazines with the readily available aldehydes 131 gives rise to the benzylic piperazines 171. Intermediates 171 are reacted with amines 37 (Z=NH), alcohols 37 (Z=O), or alkenes 37 (Z=-CH═CH₂) to give compounds 172, 173, or 174, respectively. Optionally, intermediates 174 are converted to the saturated analogs 175 under standard hydrogenation conditions.

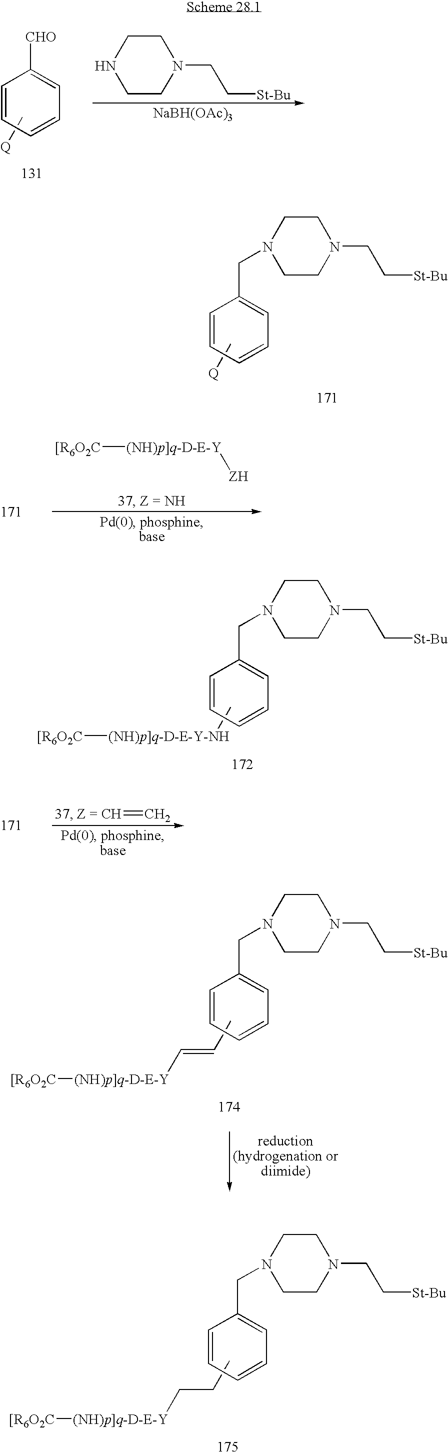

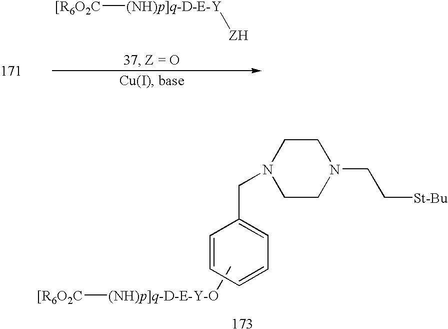

Scheme 28.2 illustrates the conversion of intermediate t-butylsulfides 172-175 to the sulfonic acids, employing a two step process involving acid-catalyzed deprotection of the t-butyl sulfide to the corresponding mercaptans, and subsequent peracid oxidation (preferably with peracetic acid or trifluoroperacetic acid) of the mercaptans to the desired sulfonic acids of Formula I-176.

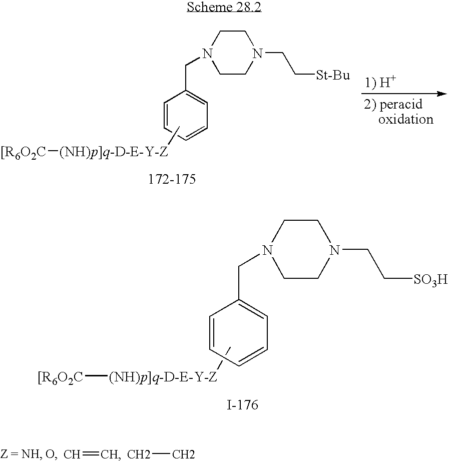

In some instances a hybrid p38-alpha kinase inhibitor is prepared which also contains an ATP-pocket binding moiety or an allosteric pocket binding moiety R₁-X-A. The synthesis of functionalized intermediates of formula R₁-X-A are accomplished as shown in Scheme 29. Readily available intermediates 177, which contain a group M capable of oxidative addition to palladium(0), are reacted with amines 178 (X═NH) under Buchwald Pd(0) amination conditions to afford 179. Alternatively amines or alcohols 178 (X═NH or O) are reacted thermally with 177 in the presence of base under nuclear aromatic substitution reaction conditions to afford 179. Alternatively, alcohols 178 (X═O) are reacted with 177 under Buchwald copper(I)-catalyzed conditions to afford 179. In cases where p=1, the carbamate of 179 is removed, preferably under acidic conditions when R₆ is t-butyl, to afford amines 180. In cases where p=0, the esters 179 are converted to the acids 181 preferably under acidic conditions when R₆ is t-butyl.

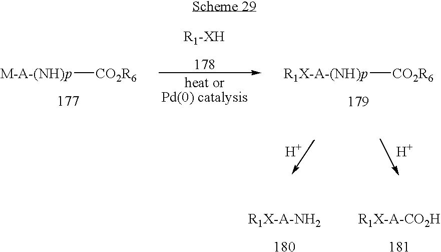

Another sequence for preparing amines 180 is illustrated in Scheme 30. Reaction of amines or alcohols 178 with nitro(hetero)arenes 182 wherein M is a leaving group, preferably M is fluoride, or M is a group capable of oxidative insertion into palladium(0), preferably M is bromo, chloro, or iodo, gives intermediates 183. Reduction of the nitro group under standard hydrogenation conditions or treatment with a reducing metal, such as stannous chloride, gives amines 180.

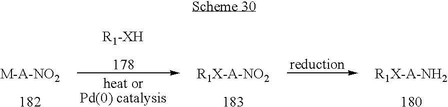

In instances when hybrid p38-alpha kinase inhibitors are prepared, compounds of Formula I-184 wherein q is 1 may be converted to amines I-185 (p=1) or acids I-186 (p=0) by analogy to the conditions described in Scheme 29. Compounds of Formula I-184 are prepared as illustrated in previous schemes 1.1, 2.1, 2.2, 3, 4, 5, 6, 7.1, 7.2, 8, 9, 10, 12, 14, 16.2, 17.2, 18, 19.1, 19.2, 19.3, 20, 21, 22, 23, 24, 25, 26, 27, or 28.2.

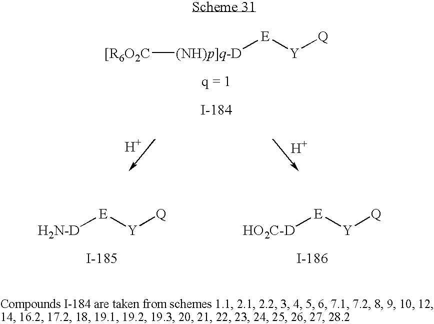

The preparation of inhibitors of Formula I which contain an amide linkage —CO—NH— connecting the oxyanion pocket binding moieties and R₁-X-A moieties are shown in Scheme 32. Treatment of acids 181 with an activating agent, preferably PyBOP in the presence of di-iso-propylethylamine, and amines I-185 gives compounds of Formula I. Alternatively, retroamides of Formula I are formed by treatment of acids I-186 with PyBOP in the presence of di-iso-propylethylamine and amines 180.

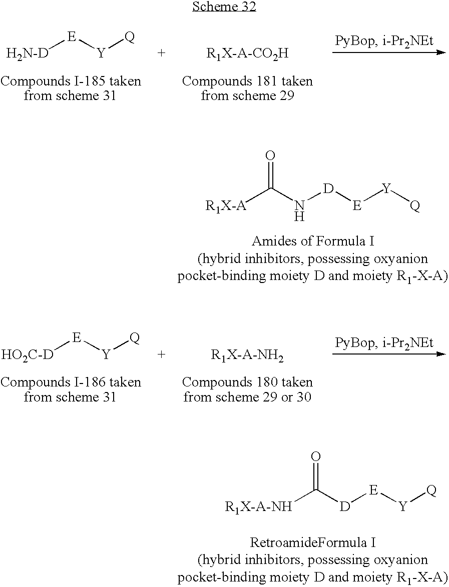

The preparation of inhibitors of Formula I which contain an urea linkage NH—CO—NH— connecting the oxyanion pocket binding moieties and the R₁-X-A moieties are shown in Scheme 33. Treatment of amines I-185 with p-nitrophenyl chloroformate and base affords carbamates 187. Reaction of 187 with amines 180 gives ureas of Formula I.

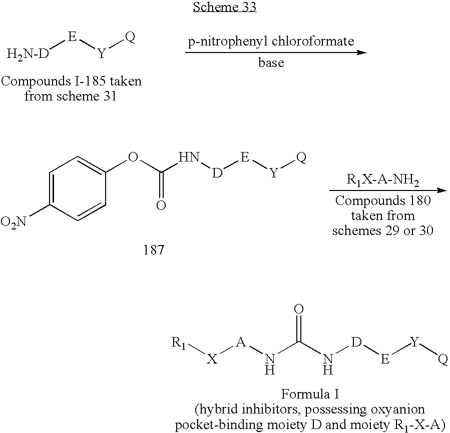

Alternatively, inhibitors of Formula I which contain an urea linkage NH—CO—NH— connecting the oxyanion pocket binding moieties and the R₁-X-A moieties are prepared as shown in Scheme 33. Treatment of amines 180 with p-nitrophenyl chloroformate and base affords carbamates 188. Reaction of 188 with amines I-185 gives ureas of Formula I.

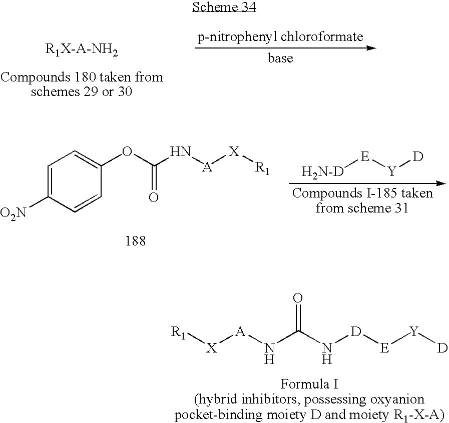

Scheme 37 illustrates the preparation of compounds wherein Q is Q-40. Readily available amine 200, wherein P is a suitable amine-protecting group or a group convertible to an amine group, is reacted with p-nitrophenyl chloroformate to give rise to carbamate 201. Intermediate 201 is reacted with a substituted amino acid ester with a suitable base to afford urea 202. Further treatment with base results in cyclization to afford hydantoin 203. The protecting group P is removed to afford the key amine-containing intermediate 204. Alternatively, if P is a nitro group, then 203 is converted to 204 under reducing conditions such as iron/HCl, tin(II) chloride, or catalytic hydrogenation. Amine 204 is converted to 205A by reaction with an isocyanate; 204 is converted to amide 205B by reaction with an acid chloride, acid anhydride, or a suitable activated carboxylic acid in the presence of a suitable base; 204 is converted to carbamate 205C by reaction with a substituted alkyl or aryl chloroformate in the presence of a suitable base.

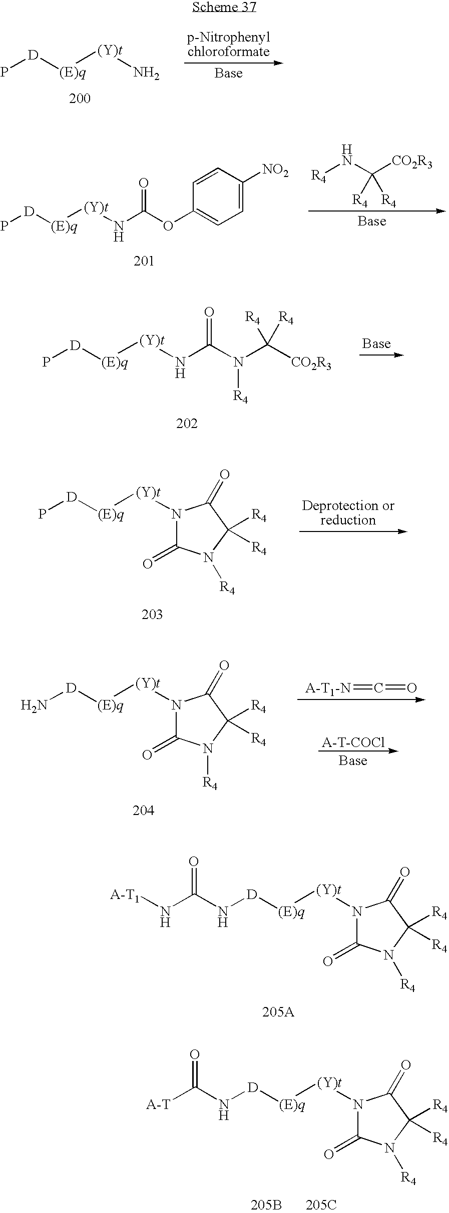

Scheme 38 illustrates the synthesis of key substituted hydrazine 210. This hydrazine can be converted into compounds of formula I using the methods previously outlined in Scheme 35. The nitrophenyl substituted amine 206 is reacted with p-nitrophenyl chloroformate to give rise to carbamate 207. Reaction of 207 with a suitable amino acid ester affords urea 208, which is cyclized under basic conditions to give hydantoin 209. Reduction of the nitro group of 209, diazotization of the resulting amine, and reduction of the diazonium salt affords key hydrazine 210.

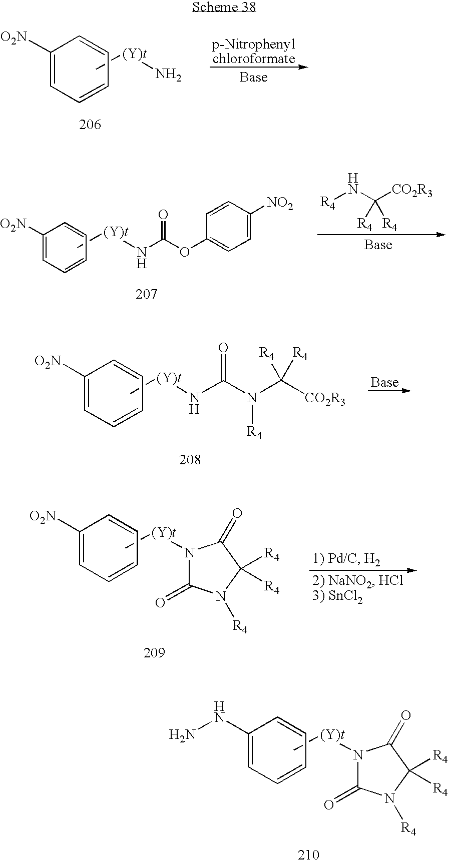

Scheme 39 illustrates the synthesis of key substituted hydrazines 213 and 216, utilized to prepare compounds of formula I wherein Q is Q-42 and G is oxygen. Nitrophenol 211 is reacted with an alpha-hydroxy acid, wherein R₄₂ is H or alkyl and R₄₃ is alkyl, under Mitsunobu reaction conditions to give 212; alternatively 211 is reacted under basic conditions with a carboxylic acid ester containing a displaceable Q, group to afford 212. Conversion of 212 to the hydrazine 213 is accomplished by standard procedures as described above.

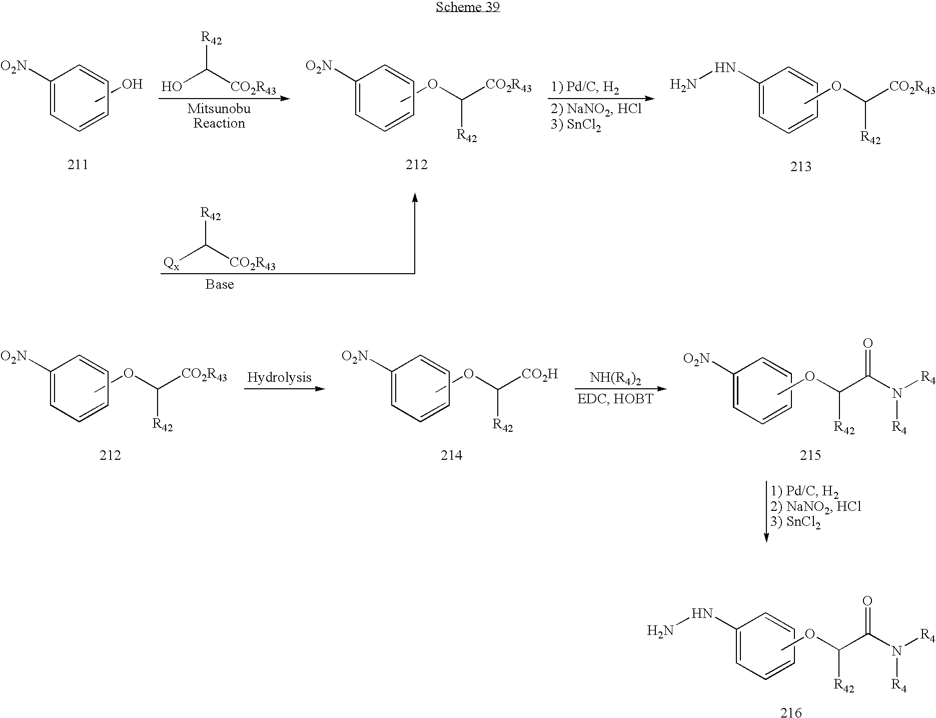

Alternatively, the ester group of 212 is hydrolyzed to afford carboxylic acid 214, which is reacted with an amine NH(R₄)₂ in the presence of a coupling reagent, preferably EDC/HOBT, to give amide 215. Conversion of 215 to the substituted hydrazine 216 is accomplished by standard procedures. Hydrazines 213 and 216 can be converted into compounds of formula I using the methods previously outlined in Scheme 35.

Scheme 40 illustrates the synthesis of key substituted hydrazines 219 and 222, utilized to prepare compounds of formula I wherein Q is Q-42 and G is methylene. Nitrophenyl bromide 217 is reacted with an alpha-beta unsaturated ester using Pd(0) catalyzed Heck reaction conditions, to afford ester 218. This intermediate is converted to the substituted hydrazine 219 by standard procedures involving concomitant reduction of the alpha-beta unsaturated bond. Alternatively, ester 218 is hydrolyzed to the carboxylic acid 220, which is reacted with an amine NH(R₄)₂ in the presence of a coupling reagent, preferably EDC/HOBT, to give amide 221. Conversion of 221 to the substituted hydrazine 222 is accomplished by standard procedures. Hydrazines 219 and 222 can be converted into compounds of formula I using the methods previously outlined in Scheme 35.

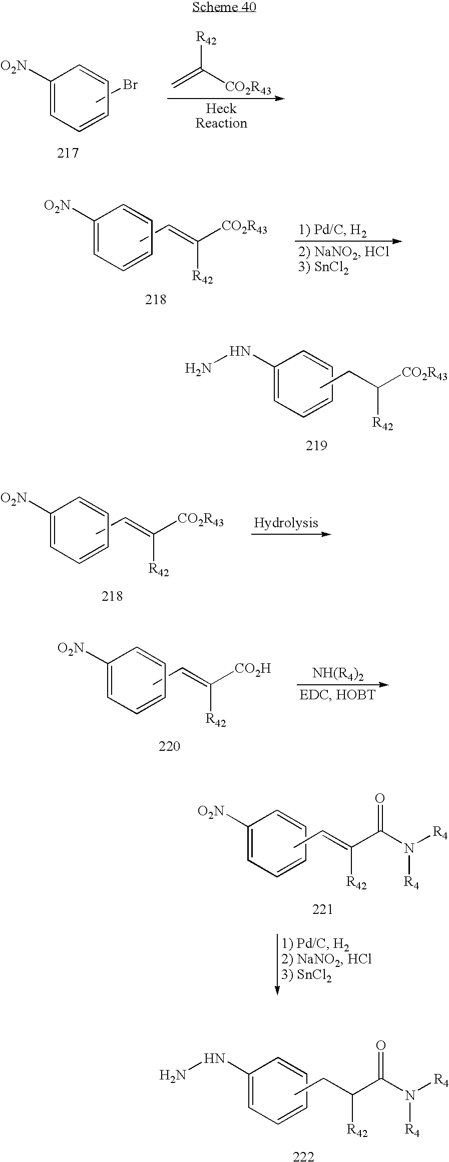

Scheme 41 illustrates an alternative synthesis of key substituted hydrazines 225 and 228, utilized to prepare compounds of formula I wherein Q is Q-42, G is methylene, and one or both of R₄₂ are carbon-containing substituents. Nitrobenzyl acetate 223 is reacted with a substituted silylketene acetal to afford ester 224. This intermediate is converted to the substituted hydrazine 225 by standard procedures. Alternatively, ester 223 is hydrolyzed to the carboxylic acid 226, which is reacted with an amine NH(R₄)₂ in the presence of a coupling reagent, preferably EDC/HOBT, to give amide 227. Conversion of 227 to the substituted hydrazine 228 is accomplished by standard procedures. Hydrazines 225 and 228 can be converted into compounds of formula I using the methods previously outlined in Scheme 35.

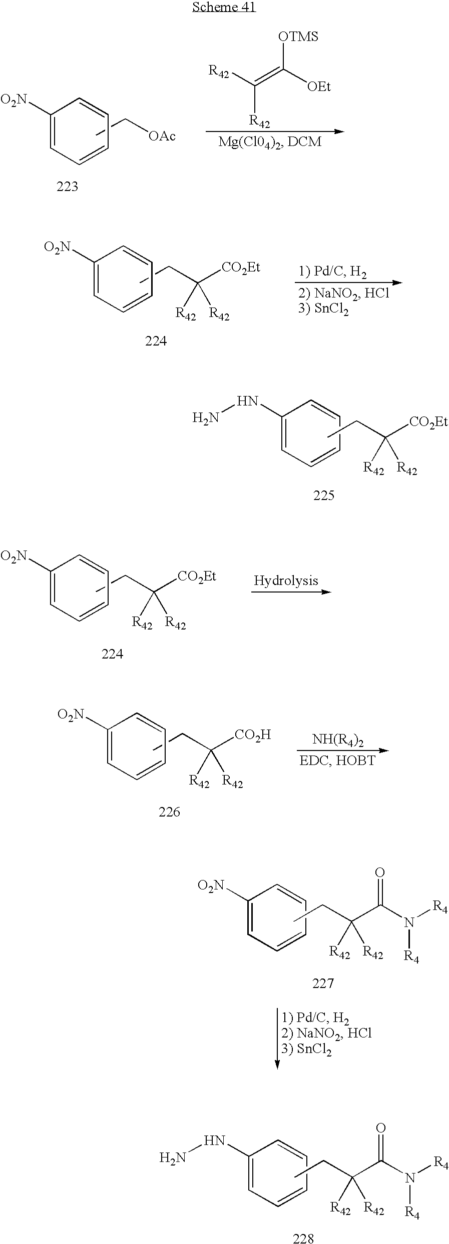

Scheme 42 illustrates an alternative synthesis of key substituted hydrazines 231 and 234, utilized to prepare compounds of formula I wherein Q is Q-42 and G is NH. Iodoaniline 229 is reacted with an alpha-keto ester under reductive amination conditions, preferably sodium triacetoxyborohydride, to afford ester 230. This intermediate is converted to the substituted hydrazine 231 by Cu(I)-catalyzed reaction with N—BOC hydrazine. Alternatively, ester 231 is hydrolyzed to the carboxylic acid 232, which is reacted with an amine NH(R₄)₂ in the presence of a coupling reagent, preferably EDC/HOBT, to give amide 233. Conversion of 233 to the substituted hydrazine 234 is accomplished by Cu(I)-catalyzed reaction with N—BOC hydrazine. Hydrazines 231 and 234 can be converted into compounds of formula I using the methods previously outlined in Scheme 35, after acid-catalyzed removal of the hydrazine N—BOC protecting group, preferably with trifluoroacetic acid or HCl-dioxane.

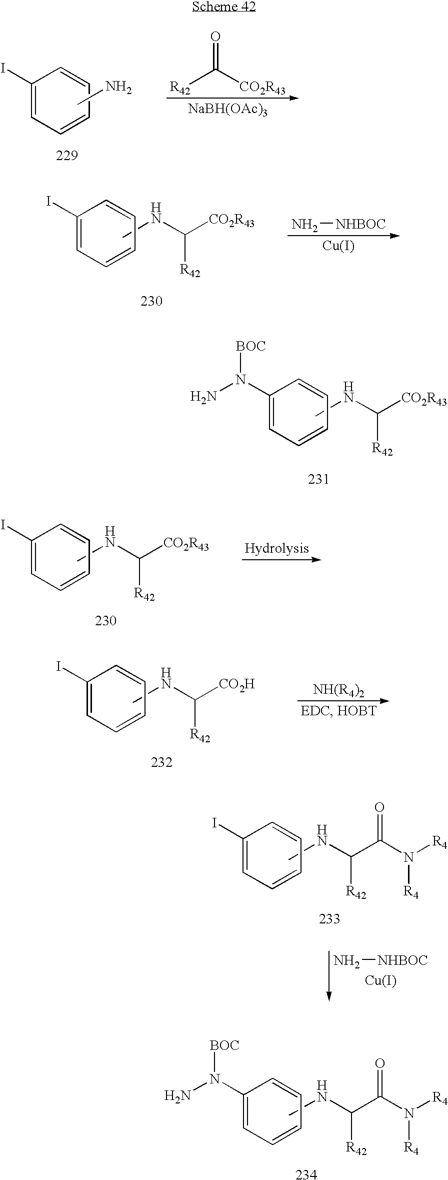

Scheme 43 illustrates an alternative synthesis of key substituted hydrazine 239, utilized to prepare compounds of formula I wherein Q is Q-42, G is oxygen, and X is taken from piperidinyl, piperazinyl, thiomorphorlino sulfone, or 4-hydroxypiperinyl. Iodophenol 235 is reacted with an alpha-hydroxy acid under Mitsunobu reaction conditions to give 236; alternatively 235 is reacted under basic conditions with a carboxylic acid ester containing a displaceable Qₓ group to afford 236. Ester 236 is hydrolyzed to the carboxylic acid 237, which is reacted with an amine X—H in the presence of a coupling reagent, preferably EDC/HOBT, to give amide 238. Conversion of 238 to the substituted hydrazine 239 is accomplished by Cu(I)-catalyzed reaction with N—BOC hydrazine. Hydrazine 239 can be converted into compounds of formula I using the methods previously outlined in Scheme 35, after acid-catalyzed removal of the hydrazine N—BOC protecting group, preferably with trifluoroacetic acid or HCl-dioxane.

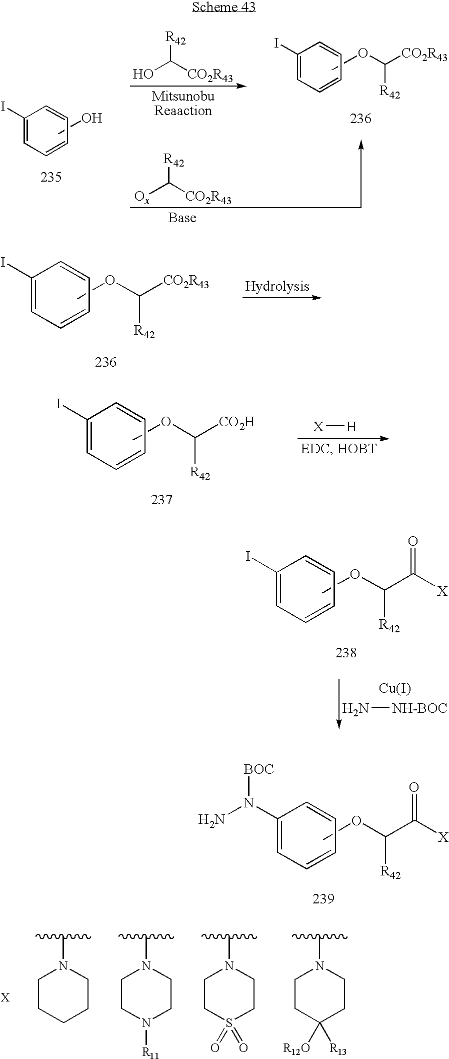

Scheme 44 illustrates an alternative synthesis of key substituted hydrazine 241, utilized to prepare compounds of formula I wherein Q is Q-42, G is NH, and X is taken from piperidinyl, piperazinyl, thiomorphorlino sulfone, or 4-hydroxypiperinyl. Carboxylic acid is reacted with an amine X—H in the presence of a coupling reagent, preferably EDC/HOBT, to give amide 240. Conversion of 240 to the substituted hydrazine 241 is accomplished by Cu(I)-catalyzed reaction with N—BOC hydrazine. Hydrazine 241 can be converted into compounds of formula I using the methods previously outlined in Scheme 35, after acid-catalyzed removal of the hydrazine N—BOC protecting group, preferably with trifluoroacetic acid or HCl-dioxane.

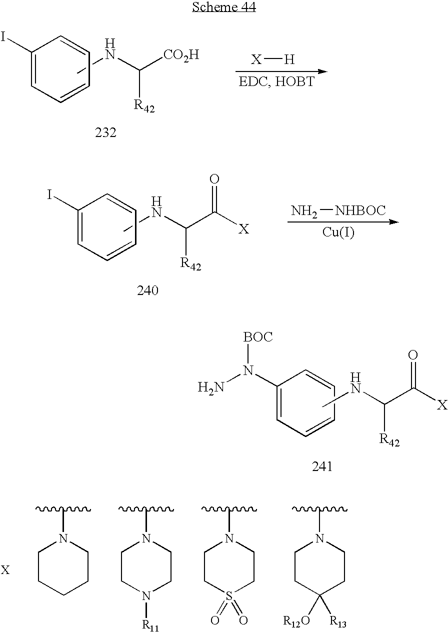

Scheme 45 illustrates an alternative synthesis of key substituted hydrazine 246, utilized to prepare compounds of formula I wherein Q is Q-42, G is methylene, and X is taken from piperidinyl, piperazinyl, thiomorphorlino sulfone, or 4 hydroxypiperinyl. Iodobenzyl acetate 242 is reacted with a substituted silylketene acetal to afford ester 243. Ester 243 is hydrolyzed to the carboxylic acid 244, which is reacted with an amine X—H in the presence of a coupling reagent, preferably EDC/HOBT, to give amide 245. Conversion of 245 to the substituted hydrazine 246 is accomplished by Cu(I)-catalyzed reaction with N—BOC hydrazine. Hydrazine 246 can be converted into compounds of formula I using the methods previously outlined in Scheme 35, after acid-catalyzed removal of the hydrazine N—BOC protecting group, preferably with trifluoroacetic acid or HCl-dioxane.

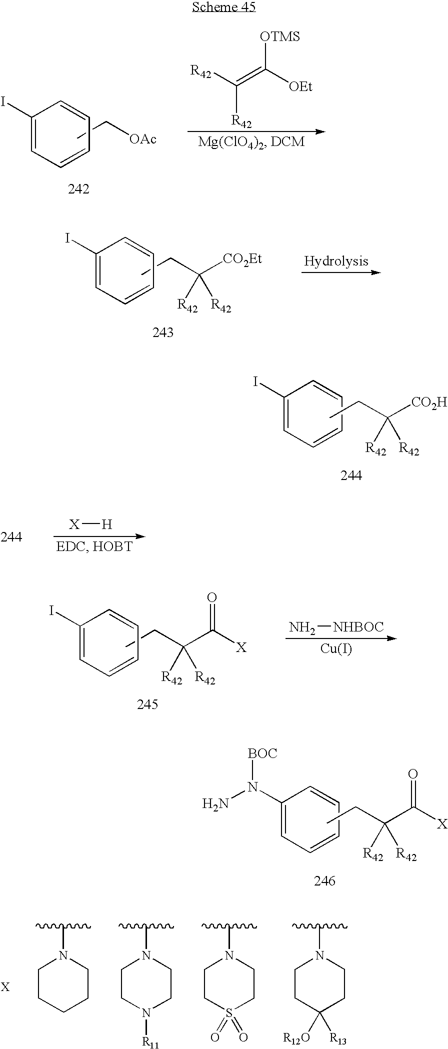

Scheme 46 illustrates an alternative synthesis of key substituted hydrazines 248, 252, and 255, utilized to prepare compounds of formula I wherein Q is Q-47 or Q-48. Nitrophenol 211 is reacted with a substituted alcohol under Mitsunobu reaction conditions to afford 247; alternatively 211 is alkylated with R₄-Qₓ, wherein Qₓ is a suitable leaving group, under basic reaction conditions, to give rise to 247. Conversion of 247 to the substituted hydrazine 248 is accomplished under standard conditions.

The nitrobenzoic acid 249 is converted to the acid fluoride 250 by reaction with a fluorinating reagent, preferably trifluorotriazine. Treatment of acid fluoride 250 with a nucleophilic fluoride source, preferably cesium fluoride and tetra-n-butylammonium fluoride, affords the alpha-alpha-difluorosubstituted carbinol 251. Conversion of 251 to the substituted hydrazine 252 is accomplished under standard conditions.

Nitrobenzaldehyde 253 is reacted with trimethylsilyltrifluoromethane (TMS-CF₃) and tetra-n-butylammonium fluoride to give rise to trifluoromethyl-substituted carbinol 254. Conversion of 254 to the substituted hydrazine 255 is accomplished under standard conditions. Hydrazines 248, 252, and 255 can be converted into compounds of formula I using the methods previously outlined in Scheme 35.

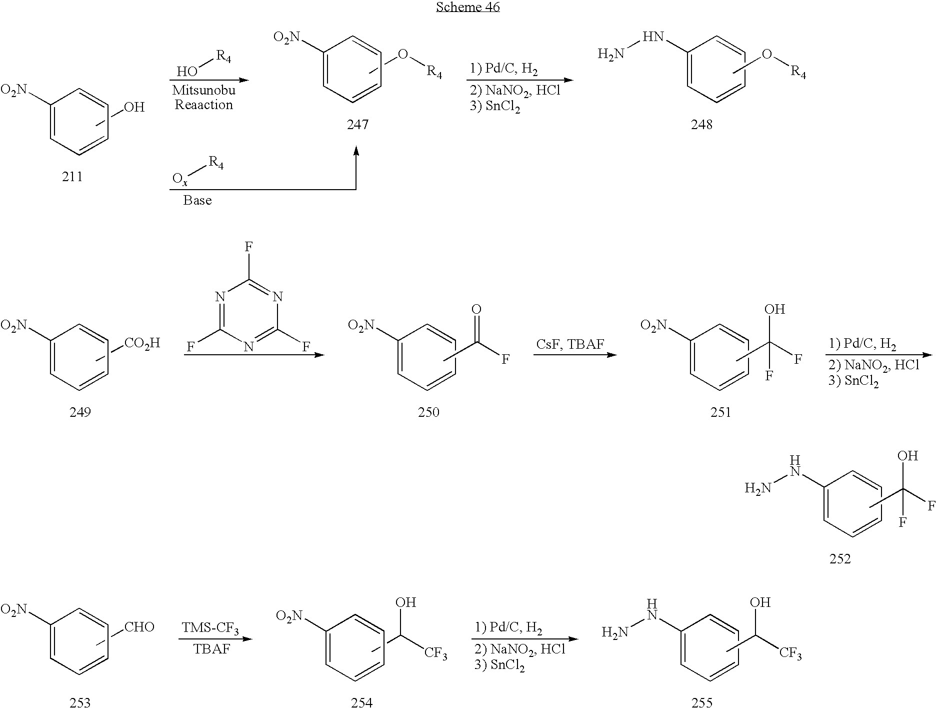

Scheme 47 illustrates the preparation of compounds of formula I wherein Q is Q-59. p-Nitrophenylcarbamate 201 is reacted with a substituted alpha-hydroxy ester with a suitable base to afford carbamate 256. Further treatment with base results in cyclization to afford oxazolidinedione 257. The protecting group P is removed to afford the key amine-containing intermediate 258; alternatively, if P is a nitro group, then 257 is converted to 258 under reducing conditions such as iron/HCl, tin(II) chloride, or catalytic hydrogenation. Amine 258 is converted to 259A by reaction with an isocyanate wherein T1 is alkylene or a direct bond connecting A and the carbonyl moiety; 258 is converted to amide 259B by reaction with an acid chloride, acid anhydride, or a suitable activated carboxylic acid in the presence of a suitable base; 258 is converted to carbamate 259C by reaction with a substituted alkyl or aryl chloroformate in the presence of a suitable base.

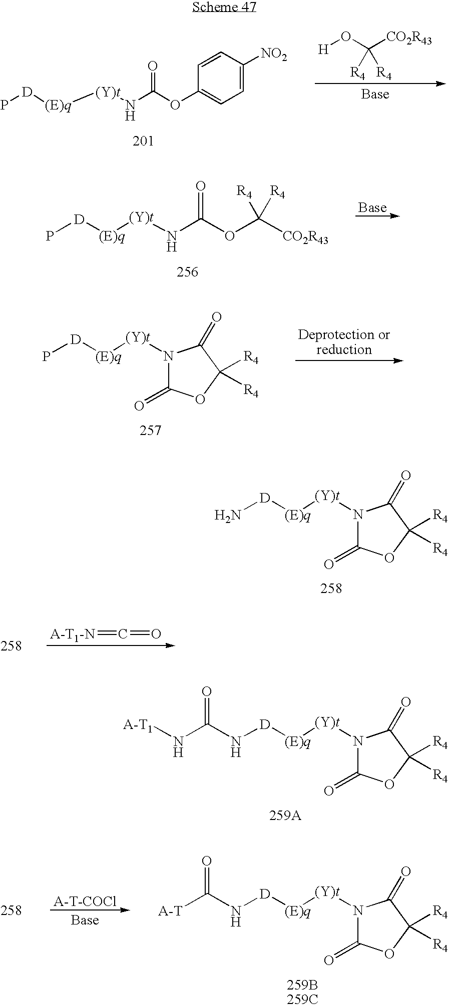

Scheme 48 illustrates an alternative approach to the preparation of compounds of formula I wherein Q is Q-59. Amine 260 is reacted with p-nitrophenylchloroformate under basic conditions to give rise to carbamate 261. This intermediate is reacted with an alpha-hydroxy ester in the presence of base to afford carbamate 262. Further treatment with base converts 262 into the oxazolidinedione 263. Conversion of 263 to the substituted hydrazine is accomplished by standard procedures. Hydrazine 264 can be converted into compounds of formula I using the methods previously outlined in Scheme 35.

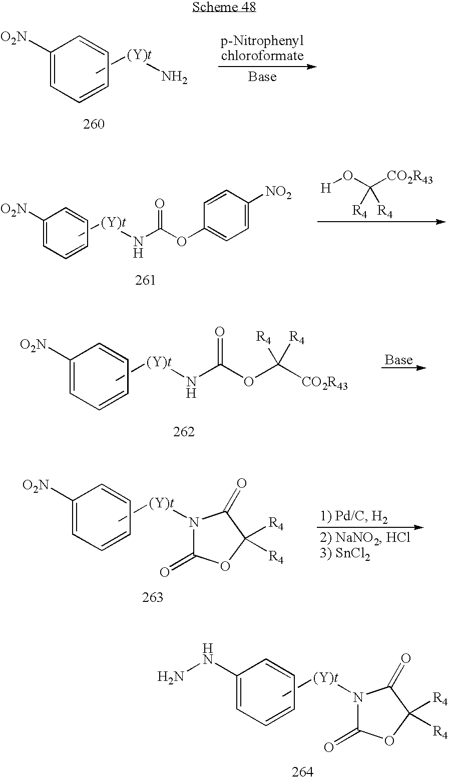

Scheme 49 illustrates thee approach to the preparation of compounds of formula I wherein Q is Q-57. Amine 265 is reacted with p-methoxybenzylisocyanate under standard conditions to give rise to urea 266. This intermediate is reacted with an oxalyl chloride in the presence of base to afford trione 267. Conversion of 267 to the substituted hydrazine 268 and removal of the p-methoxybenzyl protecting group is accomplished by standard procedures. Hydrazine 264 can be converted into compounds of formula I using the methods previously outlined in Scheme 35.

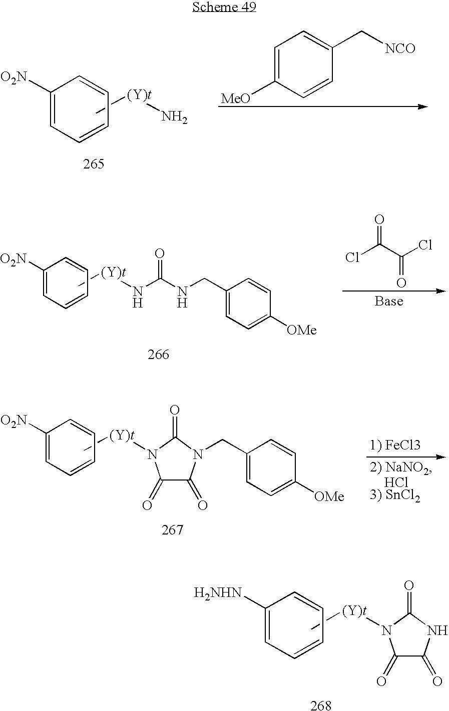

Scheme 50 illustrates an approach to the preparation of compounds of formula I wherein Q is Q-56. Amine 269 is reacted with p-methoxyberzylsulfonylchloride under standard conditions to give rise to sulfonylurea 270. This intermediate is reacted with an oxalyl chloride in the presence of base to afford the cyclic sulfonylurea 271. Conversion of 271 to the substituted hydrazine 272 and removal of the p-methoxybenzyl protecting group is accomplished by standard procedures. Hydrazine 272 can be converted into compounds of formula I using the methods previously outlined in Scheme 35.

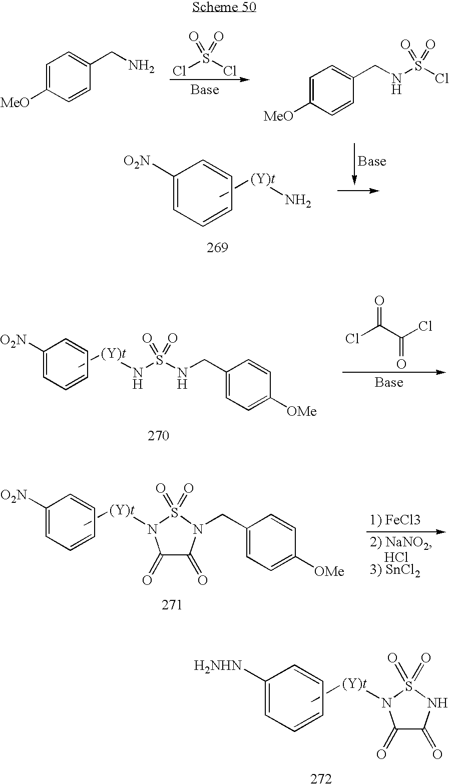

Scheme 51 illustrates an approach to the preparation of compounds of formula I wherein Q is Q-58. Amine 273 is reacted with a cyclic anhydride e.g. succinic anhydride in the presence of base under standard conditions to give rise to imide 274. Conversion of 274 to the substituted hydrazine 275 is accomplished by standard procedures. Hydrazine 275 can be converted into compounds of formula I using the methods previously outlined in Scheme 35.

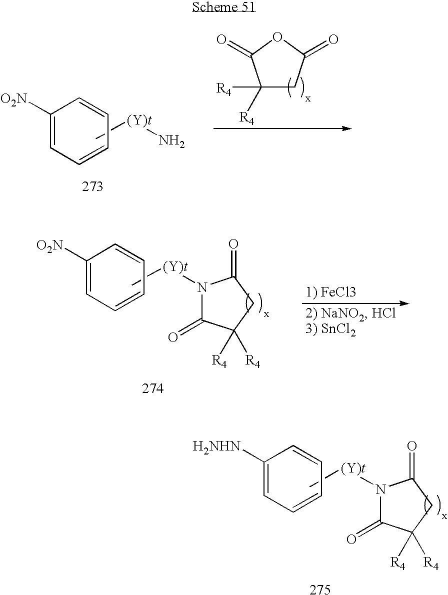

Scheme 52 illustrates an approach to the preparation of compounds of formula I wherein Q is Q-54 or Q-55. Carboxylic acid 276 is converted to protected amine 279 under standard conditions, which can be subsequently converted to hydrazine 280 by standard procedures. Hydrazine 280 can be converted into compounds of formula I using the methods previously outlined in Scheme 35 to yield protected amine 283 which is readily deprotected to yield amine 284. Reaction of amine 284 with CDI and amine (R₄)₂NH yields 285 (Q=Q-54). Reaction of amine 284 with the indicated sulfamoylchloride derivative yields 286 (Q=Q-55).

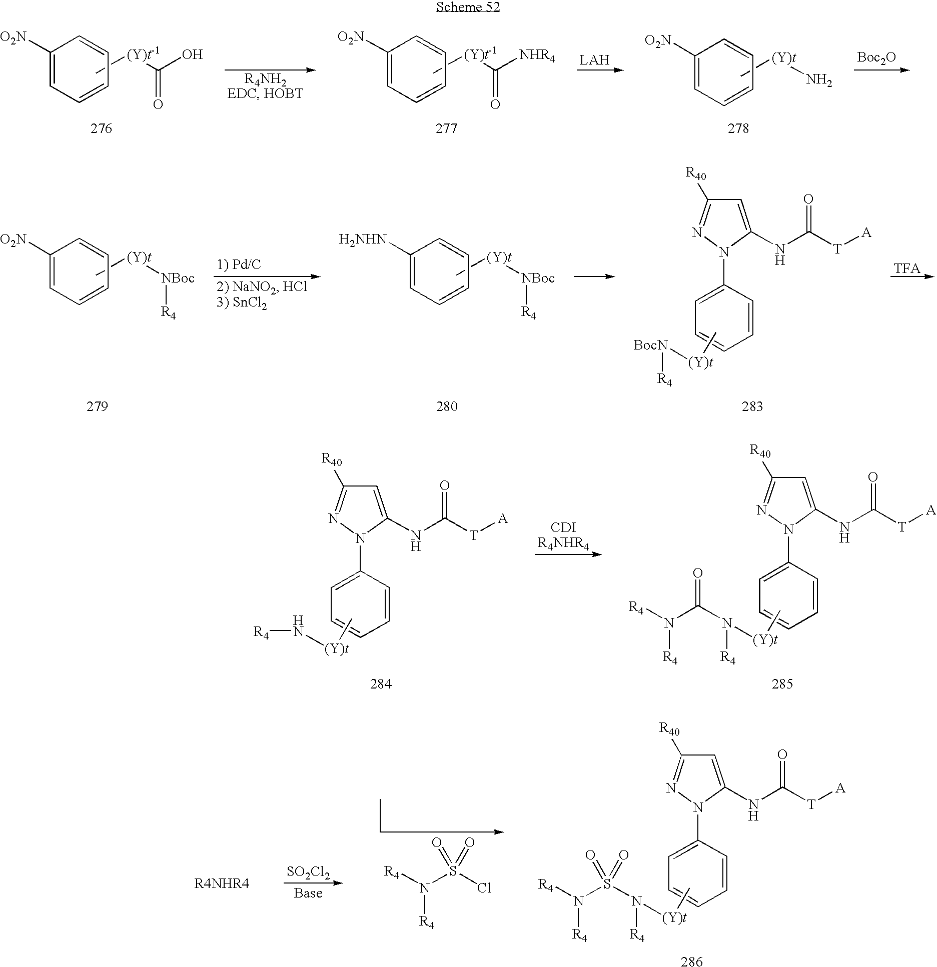

Scheme 53 illustrates an approach to the preparation of compounds of formula I wherein Q is Q-49, Q-50 or Q-51. Protected amine 287 (available by several literature procedures) is converted to deprotected hydrazine 288 is accomplished by standard procedures. Hydrazine 288 (Q=Q49) can be converted into compounds of formula I using the methods previously outlined in Scheme 35. Amine 287 can be deprotected by TFA to yield amine 289 which can be subsequently converted amide 290. Amide 290 is converted to hydrazine 291 (Q=Q-50) by standard procedures, which can be subsequently converted into compounds of formula I using the methods previously outlined in Scheme 35. Alternatively, amine 289 can be reacted with CDI and amine (R₄)₂NH to yield urea 292 (Q=Q-51). Urea 292 is converted to hydrazine 293 (Q=Q-51) by standard procedures, which can be subsequently converted into compounds of formula I using the methods previously outlined in Scheme 35.

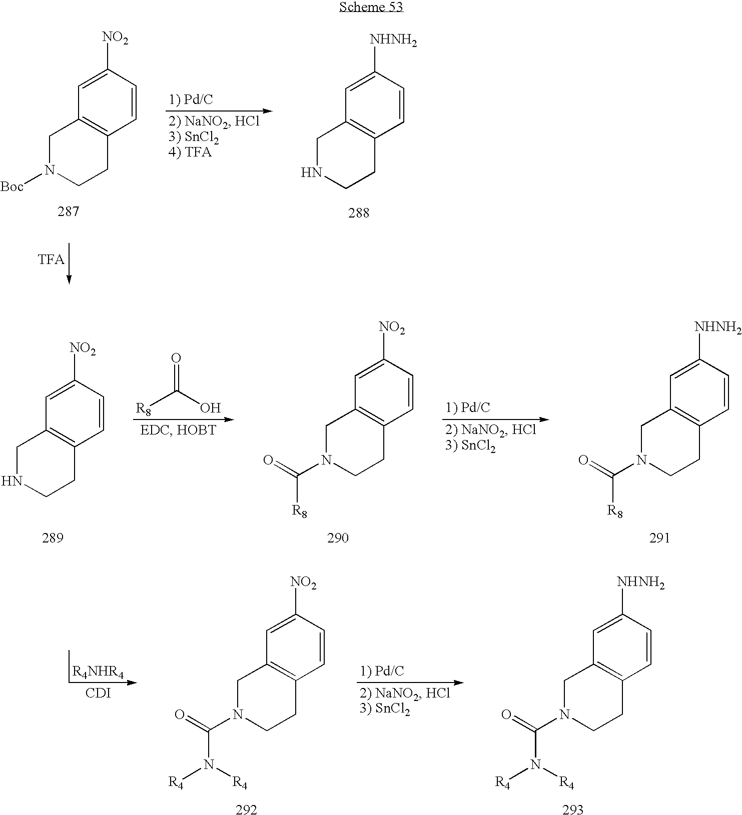

Scheme 54 illustrates an approach to the preparation of compounds of formula I wherein Q is Q-52, Q-52A, and Q-53. Protected amine 294 (available by several literature procedures) is converted to protected hydrazine 295 is accomplished by standard procedures. Hydrazine 295 (Q=Q-49) can be converted into compounds of formula I to yield protected amine 298 which is readily deprotected to yield amine 299. Reaction of amine 299 with chlorosulfonylisocyanate followed by amine (R₄)₂NH yields 300 (Q=Q-52). Alternatively, reaction of chlorosulfonylisocyanate and amine (R₄)₂NH followed by amine 299 yields 301 (Q=Q-53).

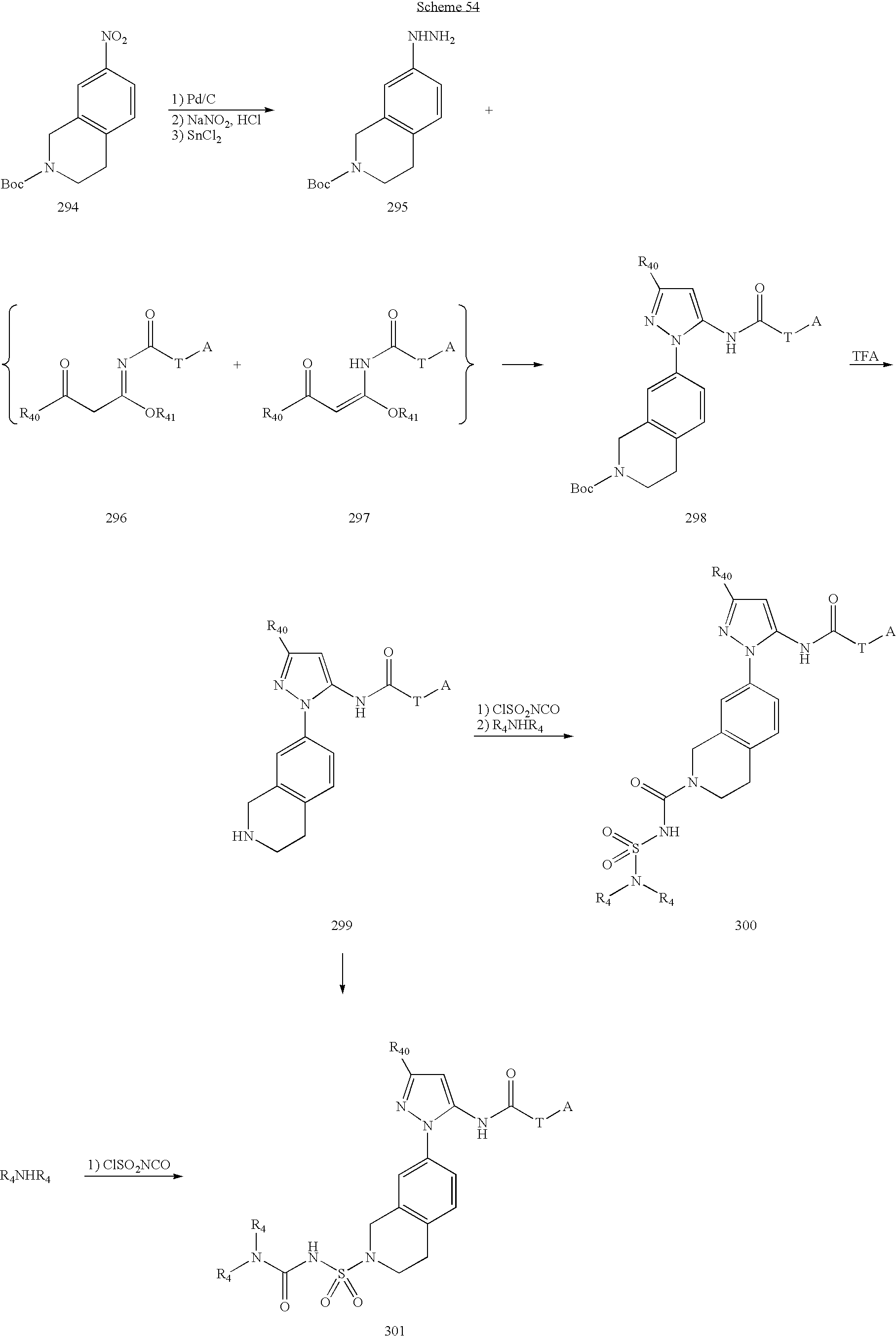

Scheme 55 illustrates an approach to the preparation of compounds of formula I wherein Q is Q-36. Amine 302 is reacted with CDI and amine R₂NH₂ to yield 303, which is reacted with chlorocarbonyl sulfenylchloride to yield thiadiazolidinedione 304. Conversion of 304 to the substituted hydrazine 305 is accomplished by standard procedures. Hydrazine 305 can be converted into compounds of formula I using the methods previously outlined in Scheme 35.

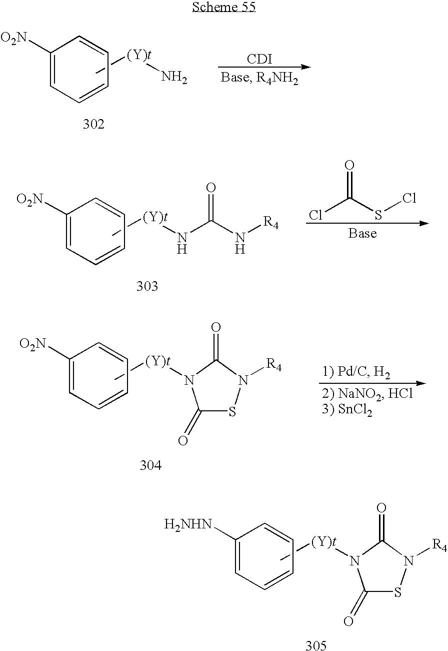

Scheme 56 illustrates an approach to the preparation of compounds of formula I wherein Q is Q-37, Q-38 or Q-39. Imides 309a, 309b, and 312 are all available via several literature methods, and are each able to be alkylated with chloride 306 to yields intermediates 307, 310 and 313 respectively. Intermediates 307, 310 and 313 are respectively converted to hydrazines 308 (Q=Q-37). 311 (Q=Q-38), and 314 (Q=Q-39) by standard procedures.

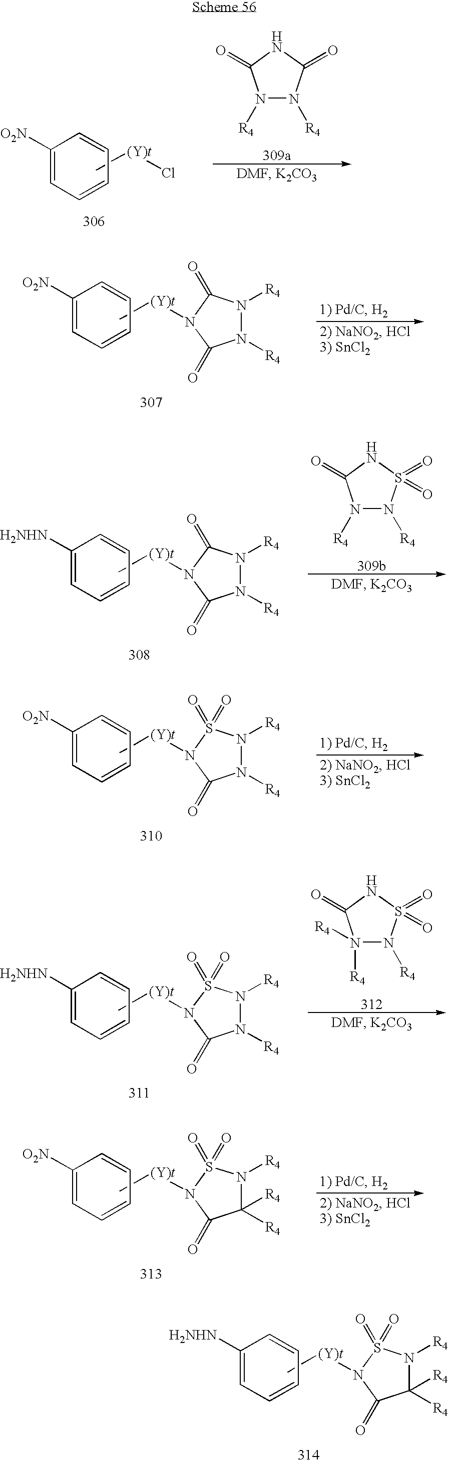

Scheme 57 illustrates an alternative preparation of compounds wherein Q is Q-39. Readily available amine 315, wherein P is a suitable amine-protecting group or a group convertible to an amine group, is reacted with SO₂Cl₂ to give rise to sulfonyl chloride 316. Intermediate 316 is reacted with a substituted amino acid ester with a suitable base to afford sulfonylurea 317. Further treatment with base results in cyclization to afford sulfohydantoin 318. The protecting group P is removed to afford the key amine-containing intermediate 319. Alternatively, if P is a nitro group, then 318 is converted to 319 under reducing conditions such as iron/HCl, tin(II) chloride, or catalytic hydrogenation. Amine 319 is converted to 320A by reaction with an isocyanate; 319 is converted to amide 320B by reaction with an acid chloride, acid anhydride, or a suitable activated carboxylic acid in the presence of a suitable base; 319 is converted to carbamate 320C by reaction with a substituted alkyl or aryl chloroformate in the presence of a suitable base.

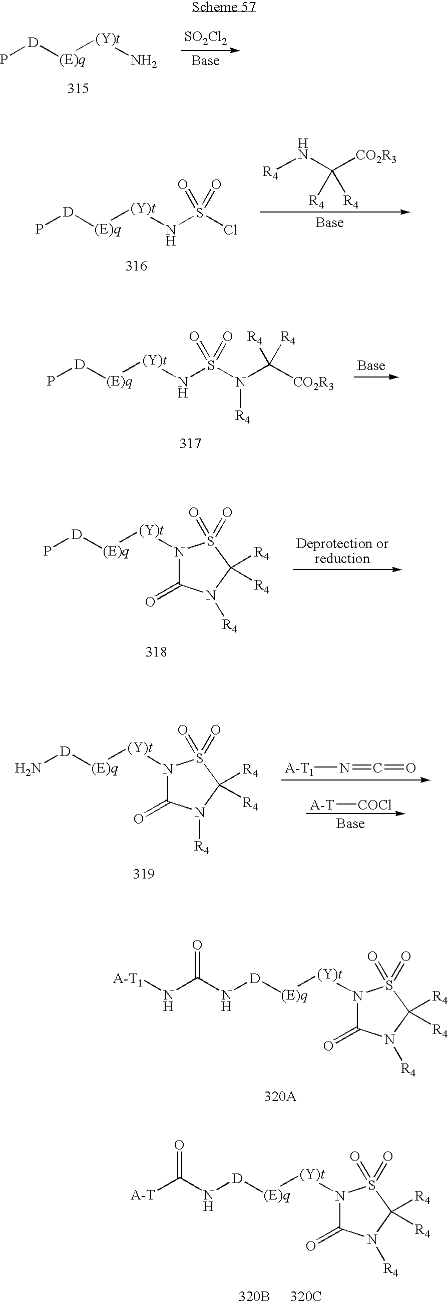

Scheme 58 illustrates an alternative synthesis of key substituted hydrazine 325 of compounds wherein Q is Q-39. This hydrazine can be converted into compounds of formula I using the methods previously outlined in Scheme 35. The amine 321 is reacted with SO₂Cl₂ to give rise to sulfonyl chloride 322. Reaction of 322 with a suitable amino acid ester affords sulfonylurea 323, which is cyclized under basic conditions to give sulfohydantoin 324. Reduction of the nitro group of 324, diazotization of the resulting amine, and reduction of the diazonium salt affords key hydrazine 325.

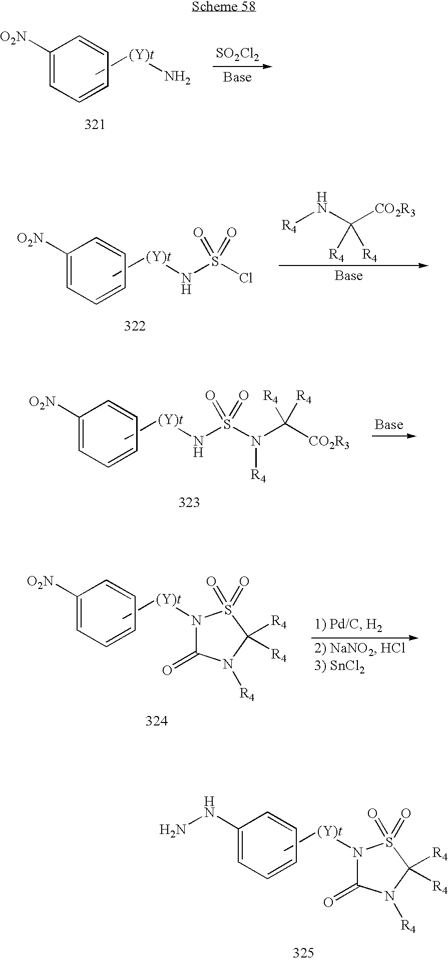

Scheme 59 illustrates an alternative preparation of compounds wherein Q is Q-38. Readily available amine 326, wherein P is a suitable amine-protecting group or a group convertible to an amine group, is reacted with SO₂Cl₂ to give rise to sulfonyl chloride 327. Intermediate 327 is reacted with a substituted hydrazide ester with a suitable base to afford sulfonylurea 328. Further treatment with base results in cyclization to afford sulfotriazaolinedione 329. The protecting group P is removed to afford the key amine-containing intermediate 330. Alternatively, if P is a nitro group, then 329 is converted to 330 under reducing conditions such as iron/HCl, tin(II) chloride, or catalytic hydrogenation. Amine 330 is converted to 331A by reaction with an isocyanate; 330 is converted to amide 331B by reaction with an acid chloride, acid anhydride, or a suitable activated carboxylic acid in the presence of a suitable base; 330 is converted to carbamate 331C by reaction with a substituted alkyl or aryl chloroformate in the presence of a suitable base.

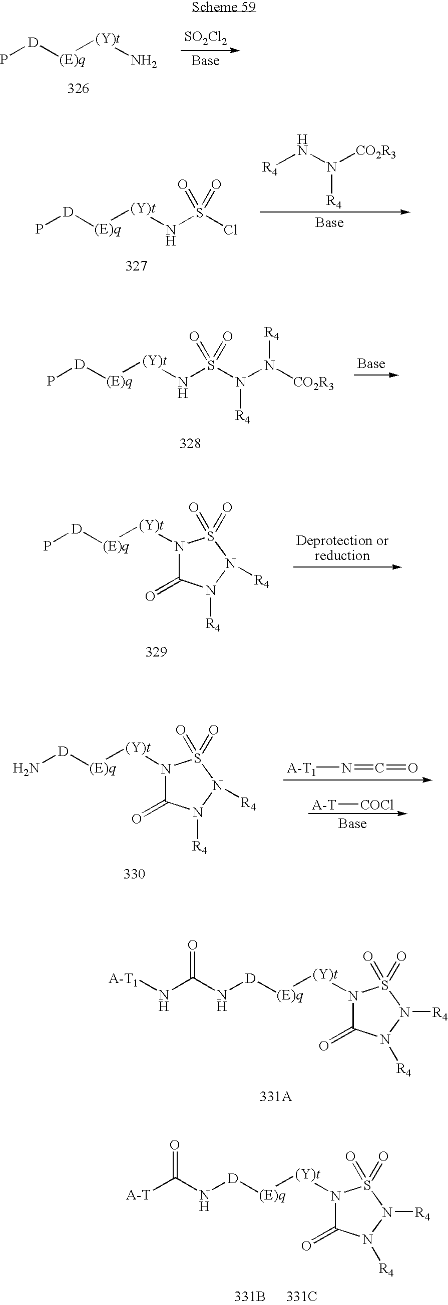

Scheme 60 illustrates an alternative synthesis of key substituted hydrazine 336 of compounds wherein Q is Q-38. This hydrazine can be converted into compounds of formula I using the methods previously outlined in Scheme 35. The amine 332 is reacted with SO₂Cl₂ to give rise to sulfonyl chloride 333. Reaction of 333 with a substituted hydrazide ester affords sulfonylurea 334, which is cyclized under basic conditions to give sulfotriazaolinedione 335. Reduction of the nitro group of 335, diazotization of the resulting amine, and reduction of the diazonium salt affords key hydrazine 336.

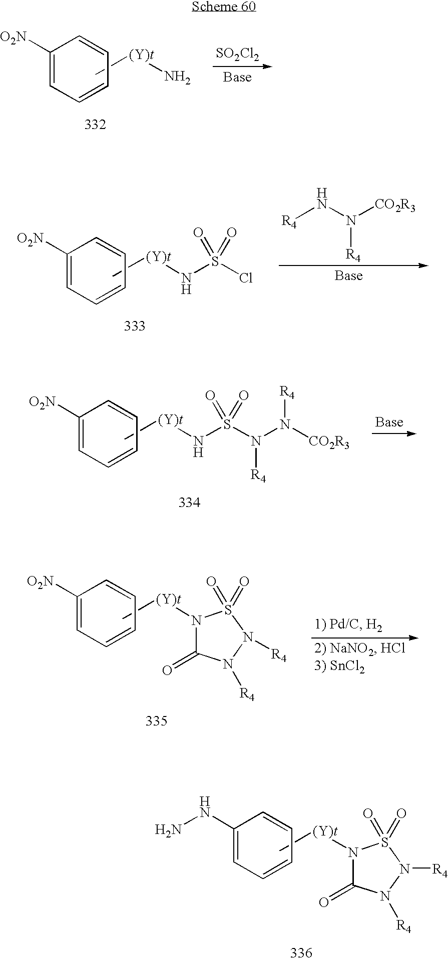

Scheme 61 illustrates the preparation of compounds wherein Q is Q-37. Readily available amine 337, wherein P is a suitable amine-protecting group or a group convertible to an amine group, is reacted with p-nitrophenyl chloroformate to give rise to carbamate 338. Intermediate 338 is reacted with a substituted amino acid ester with a suitable base to afford urea 339. Further treatment with base results in cyclization to afford triazolinedione 340. The protecting group P is removed to afford the key amine-containing intermediate 341. Alternatively, if P is a nitro group, then 340 is converted to 341 under reducing conditions such as iron/HCl, tin(II) chloride, or catalytic hydrogenation. Amine 341 is converted to 342A by reaction with an isocyanate; 341 is converted to amide 342B by reaction with an acid chloride, acid anhydride, or a suitable activated carboxylic acid in the presence of a suitable base; 341 is converted to carbamate 342C by reaction with a substituted alkyl or aryl chloroformate in the presence of a suitable base.

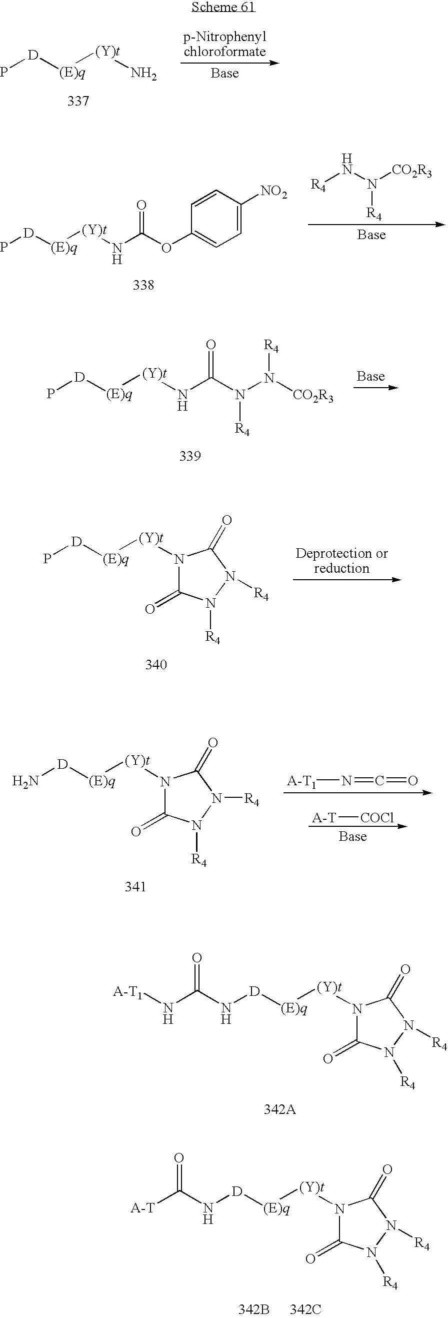

Scheme 62 illustrates an alternative synthesis of key substituted hydrazine 347 of compounds wherein Q is Q-37. This hydrazine can be converted into compounds of formula I using the methods previously outlined in Scheme 35. The nitrophenyl substituted amine 343 is reacted with p-nitrophenyl chloroformate to give rise to carbamate 344. Reaction of 344 with a suitable amino acid ester affords urea 345, which is cyclized under basic conditions to give triazolinedione 346. Reduction of the nitro group of 346, diazotization of the resulting amine, and reduction of the diazonium salt affords key hydrazine 347.

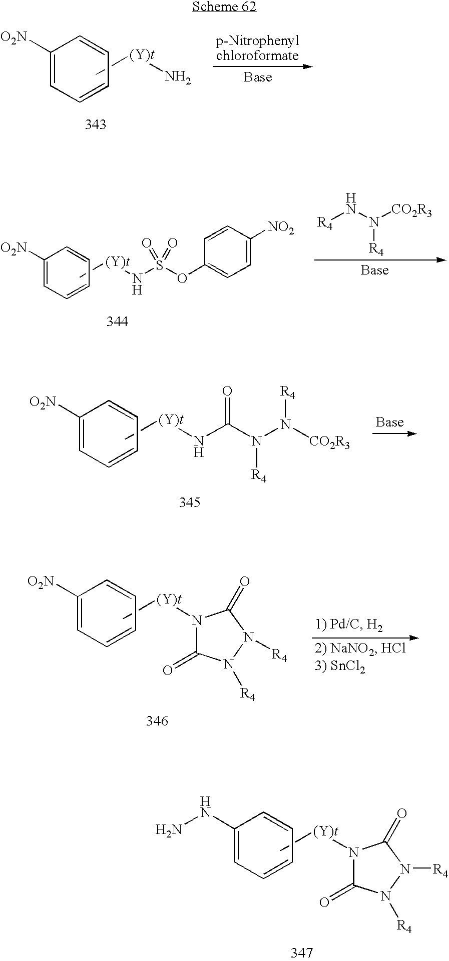

Scheme 63 illustrates the synthesis of compounds wherein Q is Q-43. Morphiline 348 is alkylated with protected bromohydrine. Removal of the alcohol protecting group yields intermediate 349, which can be oxidized to aldehyde 350. When G=NH, iodoaniline 351 is reacted with 350 under reductive amination conditions, preferably sodium triacetoxyborohydride, to afford intermediate 352. This intermediate is converted to the substituted hydrazine 353 by Cu(I)-catalyzed reaction with N—BOC hydrazine. When 0=0, iodophenol 355 is either alkylated with 354 or reacted under Mitsunobu conditions with alcohol 349 to yield intermediate 356. This intermediate is converted to the substituted hydrazine 353 by Cu(I)-catalyzed reaction with N—BOC hydrazine.

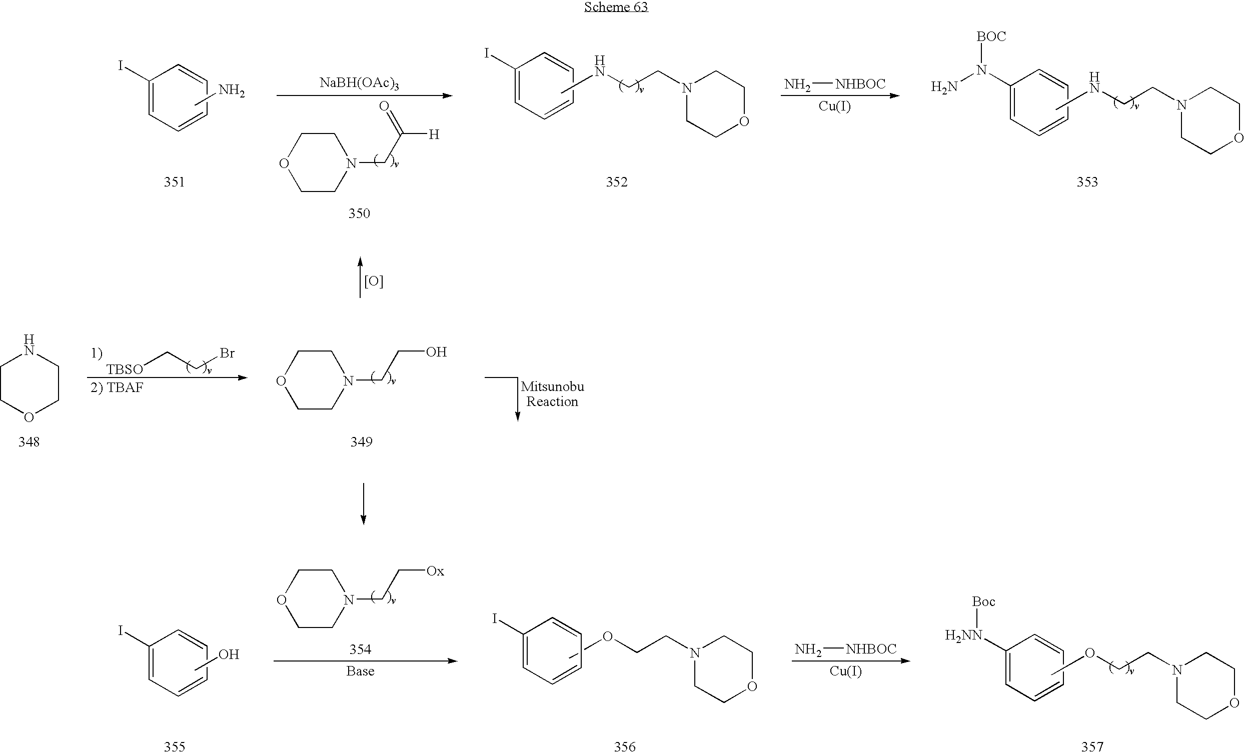

Scheme 64 illustrates the synthesis of compounds wherein Q is Q-43, G=CH₂. Nitioacid 358 (readily available by anyone with normal skills in the art) is reacted with morphiline to yield amide 359, which upon reduction to the amine and conversion of the nitro group under standard conditions results in hydrazine 360. This hydrazine can be converted into compounds of formula I using the methods previously outlined in Scheme 35.

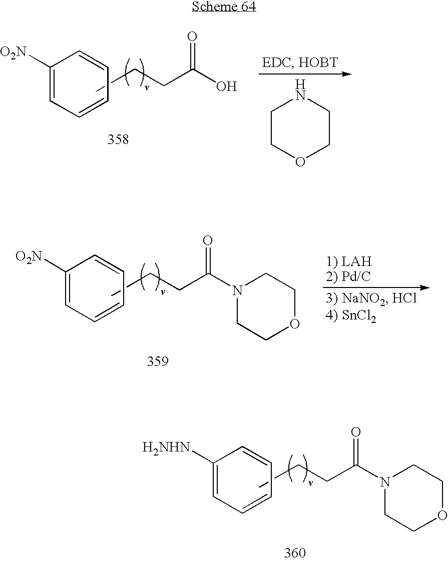

Scheme 65 illustrates the synthesis of compounds wherein Q is Q-44. N-methyl piperazine 361 is alkylated with protected bromohydrine. Removal of the alcohol protecting group yields intermediate 362, which can be oxidized to aldehyde 363. When G=NH, iodoaniline 364 is reacted with 363 under reductive amination conditions, preferably sodium triacetoxyborohydride, to afford intermediate 365. This intermediate is converted to the substituted hydrazine 366 by Cu(I)-catalyzed reaction with N—BOC hydrazine. When G=O, iodophenol 368 is either alkylated with 367 or reacted under Mitsunobu conditions with alcohol 362 to yield intermediate 369. This intermediate is converted to the substituted hydrazine 370 by Cu(I)-catalyzed reaction with N—BOC hydrazine.

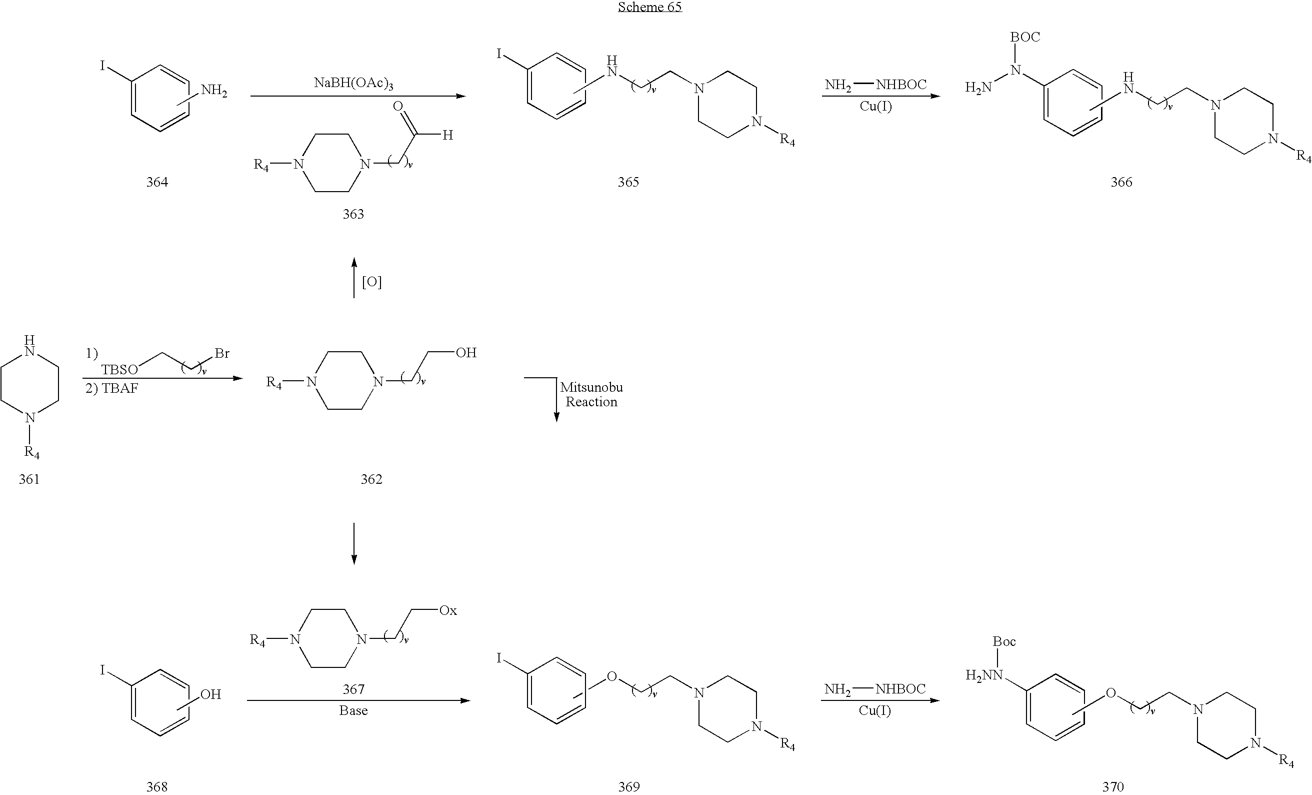

Scheme 66 illustrates the synthesis of compounds wherein Q is Q-44, G=CH₂. Nitroacid 371 (readily available by anyone with normal skills in the art) is reacted with N-methyl piperazine to yield amide 372, which upon reduction to the amine and conversion of the nitro group under standard conditions results in hydrazine 373. This hydrazine can be converted into compounds of formula I using the methods previously outlined in Scheme 35.

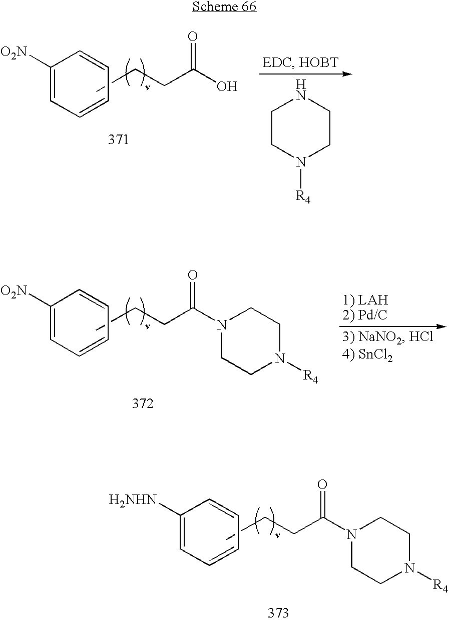

Scheme 67 illustrates the synthesis of compounds wherein Q is Q-45. Thiomorpholine sulphone 374 is alkylated with protected bromohydrine. Removal of the alcohol protecting group yields intermediate 375, which can be oxidized to aldehyde 376. When G=NH, iodoaniline 377 is reacted with 376 under reductive animation conditions, preferably sodium triacetoxyborohydride, to afford intermediate 378. This intermediate is converted to the substituted hydrazine 379 by Cu(I)-catalyzed reaction with N—BOC hydrazine. When G=O, iodophenol 380 is either alkylated with 381 or reacted under Mitsunobu conditions with alcohol 375 to yield intermediate 382. This intermediate is converted to the substituted hydrazine 383 by Cu(I)-catalyzed reaction with N—BOC hydrazine.

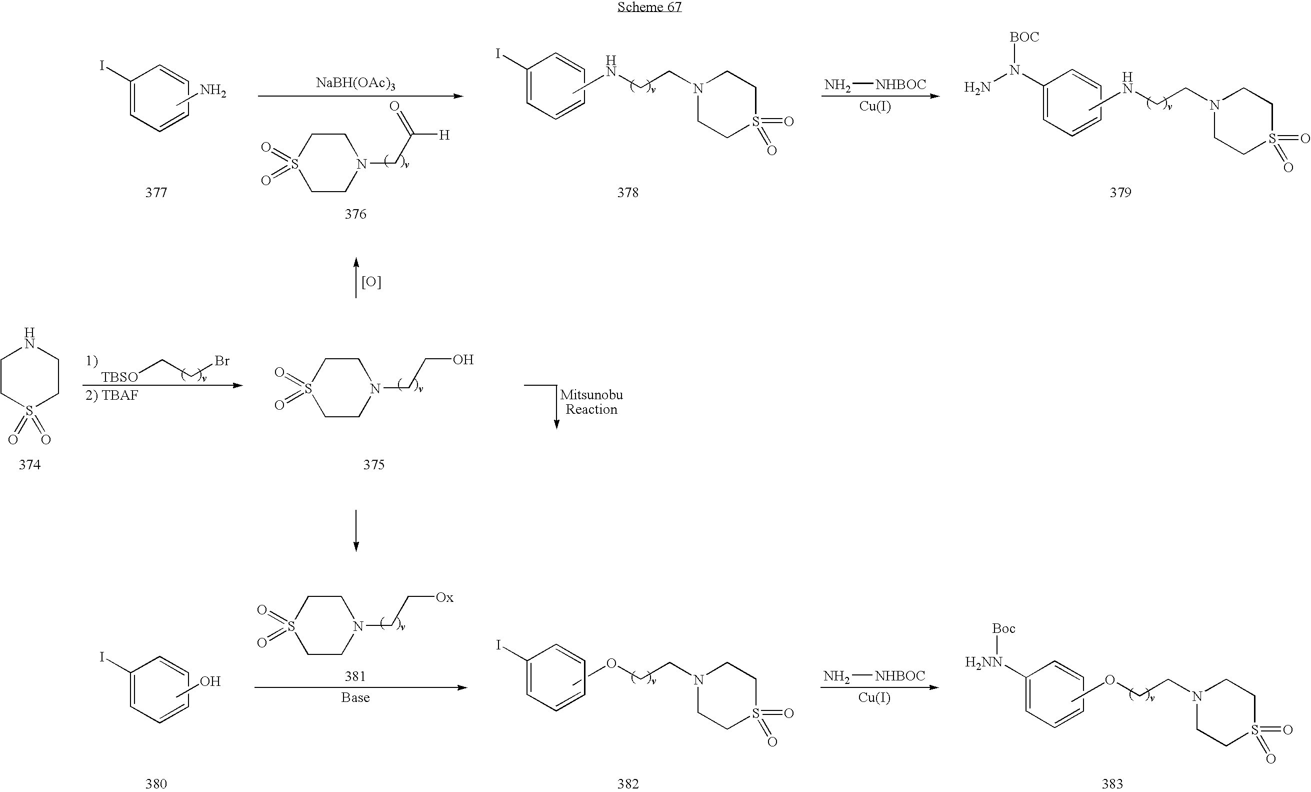

Scheme 68 illustrates the synthesis of compounds wherein Q is Q-45, G=CH₂. Nitroacid 384 (readily available by anyone with normal skills in the art) is reacted with thiomorpholine sulphone to yield amide 385, which upon reduction to the amine and conversion of the nitro group under standard conditions results in hydrazine 386. This hydrazine can be converted into compounds of formula I using the methods previously outlined in Scheme 35.

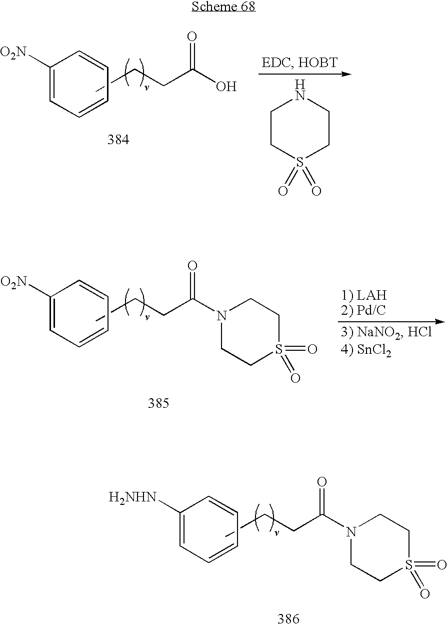

Scheme 69 illustrates the synthesis of compounds wherein Q is Q-46. Piperadine derivative 387 is alkylated with protected bromohydrine. Removal of the alcohol protecting group yields intermediate 388, which can be oxidized to aldehyde 389. When G-NH, iodoaniline 390 is reacted with 389 under reductive amination conditions, preferably sodium triacetoxyborohydride, to afford intermediate 391. This intermediate is converted to the substituted hydrazine 392 by Cu(I)-catalyzed reaction with N—BOC hydrazine. When G=O, iodophenol 393 is either alkylated with 396 or reacted under Mitsunobu conditions with alcohol 388 to yield intermediate 394. This intermediate is converted to the substituted hydrazine 395 by Cu(I)-catalyzed reaction with N—BOC hydrazine.

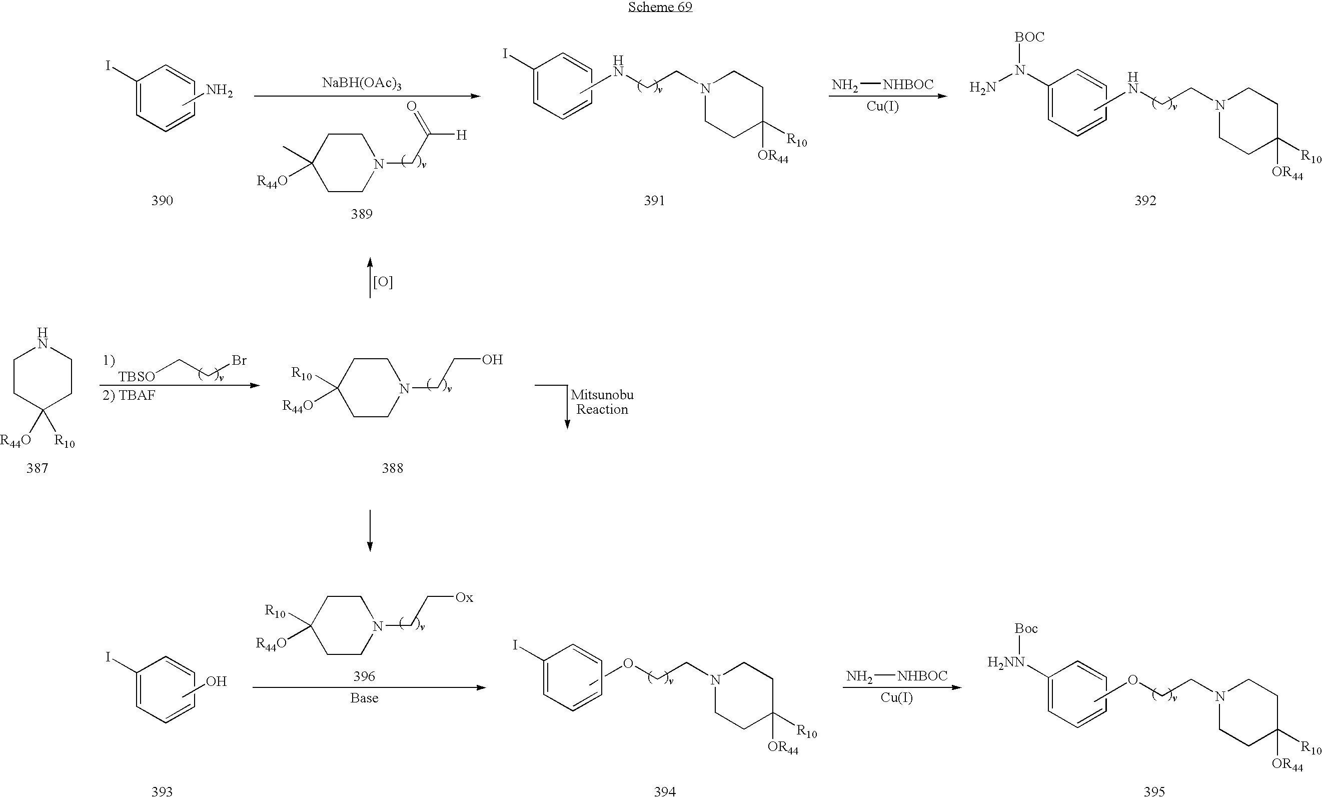

Scheme 70 illustrates the synthesis of compounds wherein Q is Q-46, G=CH₂. Nitroacid 397 (readily available by anyone with normal skills in the art) is reacted with thiomorpholine sulphone to yield amide 398, which upon reduction to the amine and conversion of the nitro group under standard conditions results in hydrazine 399. This hydrazine can be converted into compounds of formula I using the methods previously outlined in Scheme 35.

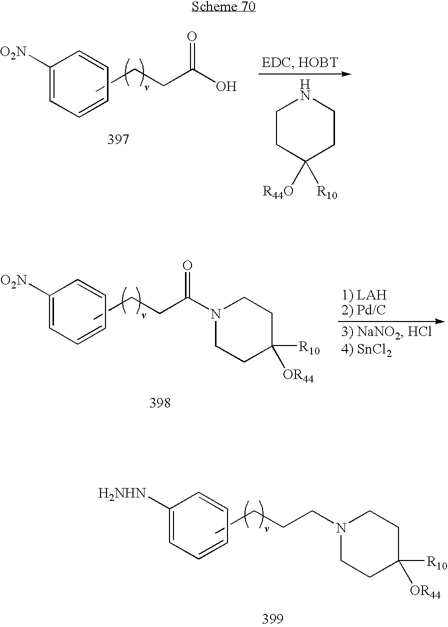

### EXAMPLES

The following examples set forth preferred methods in accordance with the invention. It is to be understood, however, that these examples are provided by way of illustration and nothing therein should be taken as a limitation upon the overall scope of the invention.

[Boc-sulfamide] aminoester (Reagent AA), 1,5,7,-trimethyl-2,4-dioxo-3-aza-bicyclo[3.3.1]nonane-7-carboxylic acid (Reagent BB), and Kemp acid anhydride (Reagent CC) was prepared according to literature procedures. See Askew et. al J. Am. Chem. Soc. 1989, 111, 1082 for further details.

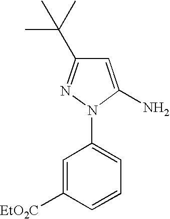

### Example A

To a solution (200 mL) of m-amino benzoic acid (200 g, 1.46 mol) in concentrated HCl was added an aqueous solution (250 mL) of NaNO₂ (102 g, 1.46 mol) at 0° C. The reaction mixture was stirred for 1 h and a solution of SnCl₂X2H₂O (662 g, 2.92 mol) in concentrated HCl (2 L) was then added at 0° C., and the reaction stirred for an additional 2 h at RT.

The precipitate was filtered and washed with ethanol and ether to yield 3-hydrazino-benzoic acid hydrochloride as a white solid.

The crude material from the previous reaction (200 g, 1.06 mol) and 4,4-dimethyl-3-oxo-pentanenitrile (146 g, 1.167 mol) in ethanol (2 L) were heated to reflux overnight. The reaction solution was evaporated in vacuo and the residue purified by column chromatography to yield ethyl 3-(3-t-butyl-5-amino-1H-pyrazol-1-yl)benzoate (Example A, 116 g, 40%) as a white solid together with 3-(5-amino-3-t-butyl-1H-pyrazol-1-yl)benzoic acid (93 g, 36%). ¹H NMR (DMSO-d₆): 8.09 (s, 1H), 8.05 (brd, J=8.0 Hz, 1H), 7.87 (brd, J=8.0 Hz, 1H), 7.71 (t, J=8.0 Hz, 1H), 5.64 (s, 1H), 4.35 (q, J=7.2 Hz, 2H), 1.34 (t, J=7.2 Hz, 3H), 1.28 (s, 9H).

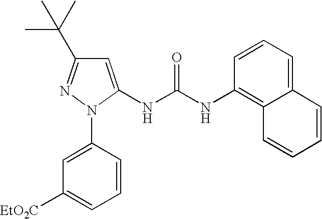

### Example B

To a solution of 1-naphthyl isocyanate (9.42 g, 55.7 mmol) and pyridine (44 mL) in THF (100 mL) was added a solution of Example A (8.0 g, 27.9 mmol) in THF (200 mL) at 0° C. The mixture was stirred at RT for 1 h, heated until all solids were dissolved, stirred at RT for an additional 3 h and quenched with H₂O (200 mL). The precipitate was filtered, washed with dilute HCl and H₂O, and dried in vacuo to yield ethyl 3-[3-t-butyl-5-(3-naphthalen-1-yl)ureido)-1H-pyrazol-1-yl]benzoate (12.0 g, 95%) as a white power. ¹H NMR (DMSO-d₆): 9.00 (s, 1H), 8.83 (s, 1H), 8.25 7.42 (m, 11H), 6.42 (s, 1H), 4.30 (q, J=7.2 Hz, 2H), 1.26 (s, 9H), 1.06 (t, J=7.2 Hz, 3H); MS (ESI) m/z: 457.10 (M+H⁺).

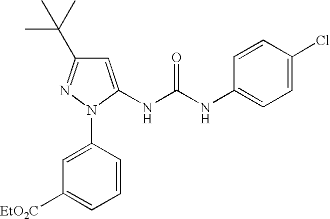

### Example C

To a solution of Example A (10.7 g, 70.0 mmol) in a mixture of pyridine (56 mL) and THF (30 mL) was added a solution of 4-nitrophenyl 4-chlorophenylcarbamate (10 g, 34.8 mmol) in THF (150 mL) at 0° C. The mixture was stirred at RT for 1 h and heated until all solids were dissolved, and stirred at RT for an additional 3 h. H₂O (200 mL) and CH₂Cl₂ (200 mL) were added, the aqueous phase separated and extracted with CH₂Cl₂ (2×100 mL). The combined organic layers were washed with 1N NaOH, and 0.1N HCl, saturated brine and dried over anhydrous Na₂SO₄. The solvent was removed in vacuo to yield ethyl 3-{3-t-butyl-5-[3-(4-chlorophenyl)ureido]-1H-pyrazol-1-yl}benzoate (8.0 g, 52%). ¹H NMR (DMSO-d₆): δ 9.11 (s, 1H), 8.47 (s, 1H), 8.06 (m, 1H), 7.93 (d, J=7.6 Hz, 1H), 7.81 (d, J=8.0 Hz, 1H), 7.65 (dd, J=8.0, 7.6 Hz, 1H), 7.43 (d, J=8.8 Hz, 2H), 7.30 (d, J=8.8 Hz, 2H), 6.34 (s, 1H), 4.30 (q, J=6.8 Hz, 2H), 1.27 (s, 9H), 1.25 (t, J=6.8 Hz, 3H); MS (ESI) m/z: 441 (M⁺+H).

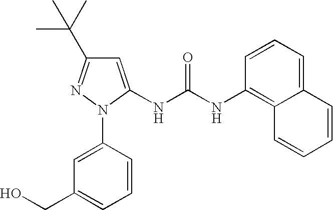

### Example D

To a stirred solution of Example B (8.20 g, 18.0 mmol) in THF (500 mL) was added LiAlH₄ powder (2.66 g, 70.0 mmol) at −10° C. under N₂. The mixture was stirred for 2 h at RT and excess LiAlH₄ destroyed by slow addition of ice. The reaction mixture was acidified to pH=7 with dilute HCl, concentrated in vacuo and the residue extracted with EtOAc. The combined organic layers were concentrated in vacuo to yield 1-{3-t-butyl-1-[3-(hydroxymethyl)phenyl]-1H-pyrazol-5-yl}-3-(naphthalen-1-yl)urea (7.40 g, 99%) as a white powder. ¹H NMR (DMSO-d₆): 9.19 (s, 1H), 9.04 (s, 1H), 8.80 (s, 1H), 8.26-7.35 (m, 11H), 6.41 (s, 1H), 4.60 (s, 2H), 1.28 (s, 9H); MS (ESI) m/z: 415 (M+H⁺).

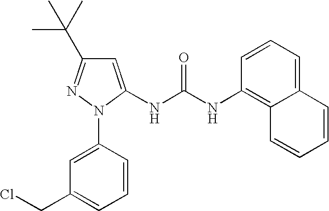

### Example E

A solution of Example C (1.66 g, 4.0 mmol) and SOCl₂ (0.60 mL, 8.0 mmol) in CH₃Cl (100 mL) was refluxed for 3 h and concentrated in vacuo to yield 1-{3-t-butyl-1-[3-chloromethyl)phenyl]-1H-pyrazol-5-yl}-3-(naphthalen-1-yl)urea (1.68 g, 97%) was obtained as white powder. ¹H NMR (DMSO-d₆): *9.26 (s, 1H), 9.15 (s, 1H), 8.42-7.41 (m, 11H), 6.40 (s, 1H), 4.85 (s, 2H), 1.28 (s, 9H). MS (ESI) m/z: 433 (M+H⁺).

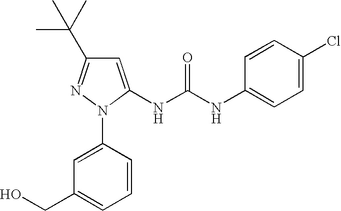

### Example F

To a stirred solution of Example C (1.60 g, 3.63 mmol) in THF (200 mL) was added LiAlH₄ powder (413 mg, 10.9 mmol) at −10° C. under N₂. The mixture was stirred for 2 h and excess LiAlH₄ was quenched by adding ice. The solution was acidified to pH=7 with dilute HCl. Solvents were slowly removed and the solid was filtered and washed with EtOAc (200+100 mL). The filtrate was concentrated to yield 1-{3-t-butyl-1-[3-hydroxymethyl)phenyl]-1H-pyrazol-5-yl}-3-(4-chlorophenyl)urea (1.40 g, 97%). ¹H NMR (DMSO-d₆): δ 9.11 (s, 1H), 8.47 (s, 1H), 7.47-7.27 (m, 8H), 6.35 (s, 1H), 5.30 (t, J=5.6 Hz, 1H), 4.55 (d, J=5.6 Hz, 2H), 1.26 (s, 9H); MS (ESI) m/z: 399 (M+H⁺).

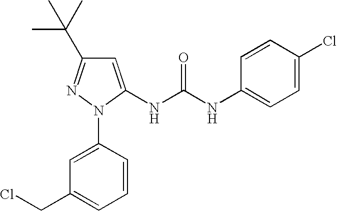

### Example G

A solution of Example F (800 mg, 2.0 mmol) and SOCl₂ (0.30 mL, 4 mmol) in CHCl₃ (30 mL) was refluxed gently for 3 h. The solvent was evaporated in vacuo and the residue was taken up to in CH₂Cl₂ (2×20 mL). After removal of the solvent, 1-{3-t-butyl-1-[3-(chloromethyl)phenyl]-1H-pyrazol-5-yl}-3-(4-chlorophenyl)urea (812 mg, 97%) was obtained as white powder. ¹H NMR (DMSO-d₆): δ 9.57 (s, 1H), 8.75 (s, 1H), 7.63 (s, 1H), 7.50-7.26 (m, 7H), 6.35 (s, 1H), 4.83 (s, 2H), 1.27 (s, 9H); MS (ESI) m/z: 417 (M+H⁺).

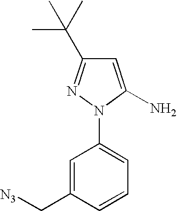

### Example H

To a suspension of LiAlH₄ (5.28 g, 139.2 mmol) in THF (1000 mL) was added Example A (20.0 g, 69.6 mmol) in portions at 0° C. under N₂. The reaction mixture was stirred for 5 h, quenched with 1 N HCl at 0° C. and the precipitate was filtered, washed by EtOAc and the filtrate evaporated to yield [3-(5-amino-3-t-butyl-1H-pyrazol-1-yl)phenyl]methanol (15.2 g, 89%). ¹H NMR (DMSO-d₆): 7.49 (s, 1H), 7.37 (m, 2H), 7.19 (d, J=7.2 Hz, 1H), 5.35 (s, 1H), 5.25 (t, J=5.6 Hz, 1H), 5.14 (s, 2H), 4.53 (d, J=5.6 Hz, 2H), 1.19 (s, 9H); MS (ESI) m/z: 246.19 (M+H⁺).

The crude material from the previous reaction (5.0 g, 20.4 mmol) was dissolved in dry THF (50 mL) and SOCl₂ (4.85 g, 40.8 mmol), stirred for 2 h at RT, concentrated in vacuo to yield 3-t-butyl-1-(3-chloromethylphenyl)-1H-pyrazol-5-amine (5.4 g), which was added to N₃ (3.93 g, 60.5 mmol) in DMF (50 mL). The reaction mixture was heated at 30° C. for 2 h, poured into H₂O (50 mL), and extracted with CH₂Cl₂. The organic layers were combined, dried over MgSO₄, and concentrated in vacuo to yield crude 3-t-butyl-1-[3-(azidomethyl)phenyl]-1H-pyrazol-5-amine (1.50 g, 5.55 mmol).

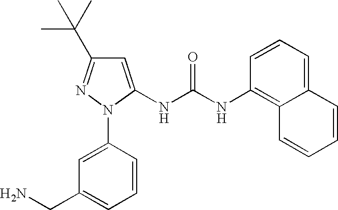

### Example I

Example H was dissolved in dry THF (10 mL) and added a THF solution (10 mL) of 1-isocyano naphthalene (1.13 g, 6.66 mmol) and pyridine (5.27 g, 66.6 mmol) at RT. The reaction mixture was stirred for 3 h, quenched with H₂O (30 mL), the resulting precipitate filtered and washed with 1N HCl and ether to yield 1-[2-(3-azidomethyl-phenyl)-5-t-butyl-2H-pyrazol-3-yl]-3-naphthalen-1-yl-urea (2.4 g, 98%) as a white solid.

The crude material from the previous reaction and Pd/C (0.4 g) in THF (30 mL) was hydrogenated under 1 atm at RT for 2 h. The catalyst was removed by filtration and the filtrate concentrated in vacuo to yield 1-{3-t-butyl-1-[3-(aminomethyl)phenyl]-1H-pyrazol-5-yl)-3-(naphthalene-1-yl)urea (2.2 g, 96%) as a yellow solid. ¹H NMR (DMSO-d₆): 9.02 (s, 1H), 7.91 (d, J=7.2 Hz, 1H), 7.89 (d, J=7.6 Hz, 2H), 7.67-7.33 (m, 9H), 6.40 (s, 1H), 3.81 (s, 2H), 1.27 (s, 9H); MS (ESI) m/z: 414 (M+H⁺).

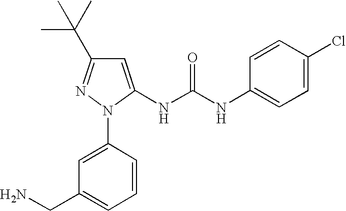

### Example J

To a solution of Example H (1.50 g, 5.55 mmol) in dry THF (10 mL) was added a THF solution (10 mL) of 4-chlorophenyl isocyanate (1.02 g, 6.66 mmol) and pyridine (5.27 g, 66.6 mmol) at RT. The reaction mixture was stirred for 3 h and then H₂O (30 mL) was added. The precipitate was filtered and washed with 1N HCl and ether to give 1-{3-t-butyl-1-[3-(aminomethyl)phenyl}-1H-pyrazol-5-yl)-3-(4-chlorophenyl)urea (2.28 g, 97%) as a white solid, which was used for next step without further purification. MS (ESI) m/z: 424 (M+H⁺).

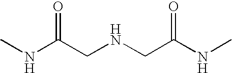

### Example K

To a solution of benzyl amine (16.5 g, 154 mmol) and ethyl bromoacetate (51.5 g, 308 mmol) in ethanol (500 mL) was added K₂CO₃ (127.5 g, 924 mmol). The mixture was stirred at RT for 3 h, was filtered, washed with EtOH, concentrated in vacuo and chromatographed to yield N-(2-ethoxy-2-oxoethyl)-N-(phenylmethyl)-glycine ethyl ester (29 g, 67%). ¹H NMR (CDCl₃): δ 7.39-7.23 (m, 5H), 4.16 (q, J=7.2 Hz, 4H), 3.91 (s, 2H), 3.54 (s, 4H), 1.26 (t, J=7.2 Hz, 6H); MS (ESI): m/e: 280 (M⁺+H).

A solution of N-(2-ethoxy-2-oxoethyl)-N-(phenylmethyl)-glycine ethyl ester (7.70 g, 27.6 mmol) in methylamine alcohol solution (25-30%, 50 mL) was heated to 50° C. in a sealed tube for 3 h, cooled to RT and concentrated in vacuo to yield N-(2-methylamino-2-oxoethyl)-N-(phenylmethyl)-glycine methylamide in quantitative yield (7.63 g). ¹H NMR (CDCl₃): δ 7.35-7.28 (m, 5H), 6.75 (br s, 2H), 3.71 (s, 2H), 3.20 (s, 4H), 2.81 (d, J=5.6 Hz, 6H); MS (ESI) m/e 250 (M+H⁺).

The mixture of N-(2-methylamino-2-oxoethyl)-N-(phenylmethyl)-glycine methylamide (3.09 g, 11.2 mmol) in MeOH (30 mL) was added 10% Pd/C (0.15 g). The mixture was stirred and heated to 40° C. under 40 psi H₂ for 10 h, filtered and concentrated in vacuo to yield N-(2-methylamino-2-oxoethyl)-glycine methylamide in quantitative yield (1.76 g). ¹H NMR (CDCl₃): δ 6.95 (br s, 2H), 3.23 (s, 4H), 2.79 (d, J=6.0, 4.8 Hz), 2.25 (br s 1H); MS (ESI) m/e 160 (M+H⁺).

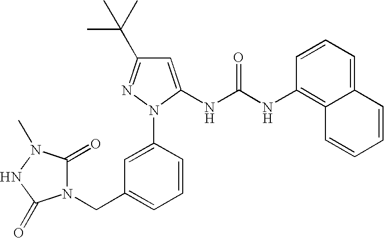

### Example 1

To a solution of 1-methyl-[1,2,4]triazolidine-3,5-dione (188 mg, 16.4 mmol) and sodium hydride (20 mg, 0.52 mmol) in DMSO (1 mL) was added Example E (86 mg, 0.2 mmol). The reaction was stirred at RT overnight, quenched with H₂O (10 mL), extracted with CH₂Cl₂, and the organic layer was separated, washed with brine, dried over Na₂SO₄ and concentrated in vacuo. The residue was purified by preparative HPLC to yield 1-(3-t-butyl-1-(3-[(1-methyl-3,5-dioxo-1,2,4-triazolidin-4-yl)methyl]phenyl)-1H-pyrazol-5-yl)-3-(naphthalene-1-yl)urea (Example 1, 14 mg). ¹H NMR (CD₃OD): *7.88-7.86 (m, 2H), 7.71-7.68 (m, 2H), 7.58 (m, 2H), 7.60-7.42 (m, 5H), 6.49 (s, 1H), 4.85 (s, 1H), 1.34 (s, 9H), 1.27 (s, 6H); MS (ESI) m/z: 525 (M+H⁺).

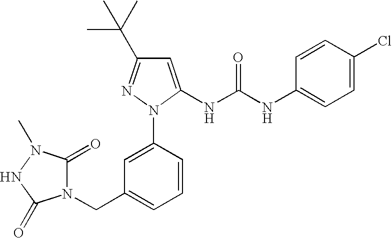

### Example 2

The title compound was synthesized in a manner analogous to Example 1, utilizing Example G to yield 1-(3-t-butyl-1-{3-[(1-methyl-3,5-dioxo-1,2,4-triazolidin-4-yl)methyl]phenyl}-1H-pyrazol-5-yl)-3-(4-chlorophenyl)urea ¹H NMR (CD₃OD): *7.2˜7.5 (m, 7H), 6.40 (s 1H), 4.70 (s, 2H), 2.60 (d, J=14 Hz, 2H), 1.90 (m, 1H), 1.50 (m, 1H), 1.45 (s, 9H), 1.30 (m, 2H), 1.21 (s, 3H), 1.18 (s, 6H); MS (ESI) m/z: 620 (M+H⁺).

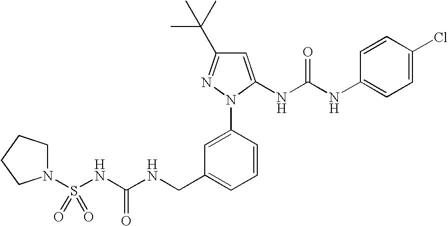

### Example 3

A mixture of compound 1,1-Dioxo-[1,2,5]thiadiazolidin-3-one (94 mg, 0.69 mmol) and NaH (5.5 mg, 0.23 mmol) in THF (2 mL) was stirred at −10° C. under N₂ for 1 h until all NaH was dissolved. Example E (100 mg, 0.23 mmol) was added and the reaction was allowed to stir at RT overnight, quenched with H₂O, and extracted with CH₂Cl₂. The combined organic layers were concentrated in vacuo and the residue was purified by preparative HPLC to yield 1-(3-t-butyl-1-{[3-(1,1,3-trioxo-[1,2,5]thiadiazolidin-2-yl)methyl]phenyl}-1H-pyrazol-5-yl)-3-(naphthalen-1-yl)urea (18 mg) as a white powder. ¹H NMR (CD₃OD): *7.71-7.44 (m, 11H), 6.45 (s, 1H), 4.83 (s, 2H), 4.00 (s, 2H), 1.30 (s, 9H). MS (ESI) m/z: 533.40 (M+H⁺).

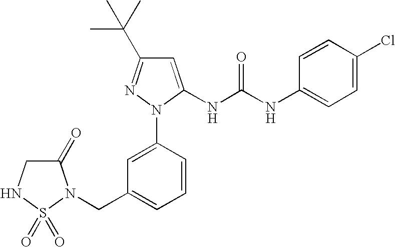

### Example 4

The title compound was obtained in a manner analogous to Example 3 utilizing Example G, to yield 1-{3-t-butyl-1-([3-(1,1,3-trioxo-[1,2,5]thiadiazolidin-2-yl)methyl]phenyl}-1H-pyrazol-5-yl)-3-(4-chlorophenyl)urea. ¹H NMR (CD₃OD): *7.38-7.24 (m, 8H), 6.42 (s, 1H), 4.83 (s, 2H), 4.02 (s, 2H), 1.34 (s, 9H). MS (ESI) m/z: 517 (M+H⁺).

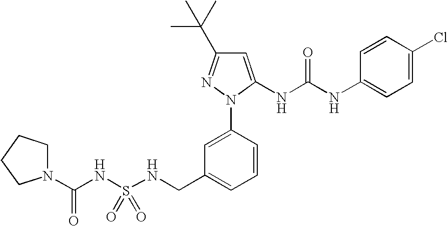

### Example 5

To a stirred solution of chlorosulfonyl isocyanate (19.8 μL, 0.227 mmol) in CH₂Cl₂ (0.5 mL) at 0° C. was added pyrrolidine (18.8 μL, 0.227 mmol) at such a rate that the reaction solution temperature did not rise above 5° C.

After stirring for 1.5 h, a solution of Example J (97.3 mg, 0.25 mmol) and Et₃N (95 μL, 0.678 mmol) in CH₂Cl₂ (1.5 mL) was added at such a rate that the reaction temperature didnt rise above 5° C. When the addition was completed, the reaction solution was warmed to RT and stirred overnight. The reaction mixture was poured into 10% HCl, extracted with CH₂Cl₂, the organic layer washed with saturated NaCl, dried over MgSO₄, and filtered. After removal of the solvents, the crude product was purified by preparative HPLC to yield 1-(3-t-butyl-1-[[3-N-[[(1-pyrrolidinylcarbonyl)amino]sulphonyl]aminomethyl]phenyl]-1H-pyrazol-5-yl)-3-(4-chlorophenyl)urea. ¹H NMR (CD₃OD): *7.61 (s, 1H), 7.43-7.47 (m, 3H), 7.23-7.25 (dd, J=6.8 Hz, 2H), 7.44 (dd, J=6.8 Hz, 2H), 6.52 (s, 1H), 4.05 (s, 2H), 3.02 (m, 4H), 1.75 (m, 4H), 1.34 (s, 9H); MS (ESI) m/z: 574.00 (M+H⁺).

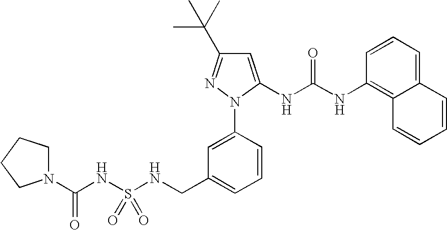

### Example 6

The title compound was made in a manner analogous to Example 5 utilizing Example I to yield 1-(3-t-butyl-1-[[3-N-[[(1-pyrrolidinylcarbonyl)amino]sulphonyl]-aminomethyl]-phenyl]-1H-pyrazol-5-yl)-3-(naphthalen-1-yl)urea. ¹HNMR (CDCl₃): *7.88 (m, 2H), 7.02-7.39 (m, 2H), 7.43-7.50 (m, 7H), 6.48 (s, 1H), 4.45 (s, 1H), 3.32-3.36 (m, 4H), 1.77-1.81 (m, 4H), 1.34 (s, 9H); MS (ESI) m/z: 590.03 (M+H⁺).

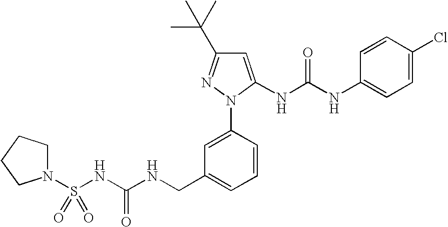

### Exampkle 7

To a stirred solution of chlorosulfonyl isocyanate (19.8μΛ, 0.227μμoλ) τν XH2Xλ₂ (0.5μΛ) ατ 0° C., was added Example J (97.3 mg, 0.25 mmol) at such a rate that the reaction solution temperature did not rise above 5° C. After being stirred for 1.5 h, a solution of pyrrolidine (18.8 μL, 0.227 mmol) and Et₃N (95 μL, 0.678 mmol) in CH₂Cl₂ (1.5 mL) was added at such a rate that the reaction temperature didnt rise above 5° C. When addition was completed, the reaction solution was warmed to RT and stirred overnight. The reaction mixture was poured into 10% HCl, extracted with CH₂Cl₂, the organic layer was washed with saturated NaCl, dried over Mg₂SO₄, and filtered. After removal of the solvents, the crude product was purified by preparative HPLC to yield 1-(3-t-butyl-1-[[3-N-[[(1-pyrrolidinylsulphonyl)amino]carbonyl]aminomethyl]phenyl]-1H-pyrazol-5-yl)-3-(4-chlorophenyl)urea. ¹HNMR (CDCl₃): *7.38 (m, 1H), 7.36-7.42 (m, 3H), 7.23 (d, J=8.8 Hz, 2H), 7.40 (d, J=8.8 Hz, 2H), 6.43 (s, 1H), 4.59 (s, 1H), 4.43 (s, 2H), 1.81 (s, 2H), 1.33 (s, 9H); MS (ESI) m/z: 574.10 (M+H⁺).

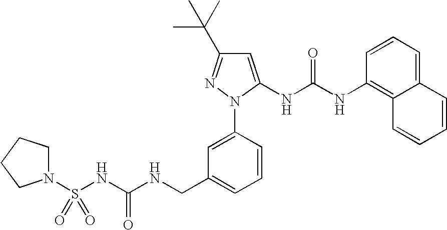

### Example 8

The title compound was made in a manner analogous to Example 7 utilizing Example I to yield 1-(3-t-butyl-1-[[3-N-[[(1-pyrrolidinylsulphonyl)amino]-carbonyl]aminomethyl]-phenyl]-1H-pyrazol-5-yl)-3-(naphthalen-1-yl)urea. ¹HNMR (CDCl₃): *7.88 (m, 2H), 7.02-7.39 (m, 2H), 7.43-7.50 (m, 7H), 6.48 (s, 1H), 4.45 (s, 1H), 3.32-3.36 (m, 4H), 1.77-1.81 (m, 4H), 1.34 (s, 9H); MS (ESI) m/z: 590.03

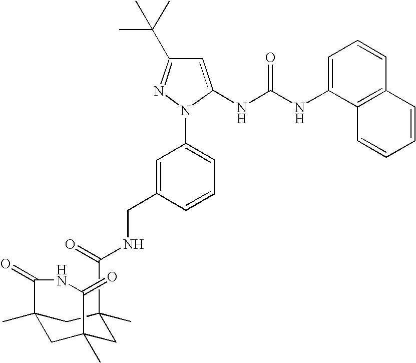

### Example 9

To a solution of Reagent BB (36 mg, 0.15 mmol), Example I (62 mg, 0.15 mmol), HOBt (40 mg, 0.4 mmol) and NMM (0.1 mL, 0.9 mmol) in DMF (10 mL) was added EDCI (58 mg, 0.3 mmol). After being stirred overnight, the mixture was poured into water (15 mL) and extracted with EtOAc (35 mL). The organic layers were combined, washed with brine, dried with Na₂SO₄, and concentrated in vacuo. The residue was purified by preparative TLC to yield 1,5,7-trimethyl-2,4-dioxo-3-azabicyclo[3.3.1]nonane-7-carboxylic acid 3-[3-t-butyl-5-(3-naphthalen-1-yl-ureido)-pyrazol-1-yl]benzylamide (22 mg). ¹H NMR (CDCl₃): *8.40 (s, 1H), 8.14 (d, J=8.0 Hz, 2H), 7.91 (s, 1H), 7.87 (s, 1H), 7.86 (d, J=7.2 Hz, 1H), 7.78 (d, J=7.6 Hz, 1H), 7.73 (d, J=8.4 Hz, 1H), 7.69 (d, J=8.4 Hz, 1H), 7.57-7.40 (m, 4H), 7.34 (d, J=7.6 Hz, 1H), 6.69 (s, 1H), 6.32 (t, J=5.6 Hz, 1H), 5.92 (brs, 1H), 4.31 (d, J=5.6 Hz, 2H), 2.37 (d, J=14.8 Hz, 2H), 1.80 (d, J=13.2 Hz, 1H), 1.35 (s, 9H), 1.21 (d, J=13.2 Hz, 1H), 1.15 (s, 3H), 1.12 (d, J=12.8 Hz, 2H), 1.04 (s, 6H); MS (ESI) m/z: 635 (M+H⁺).

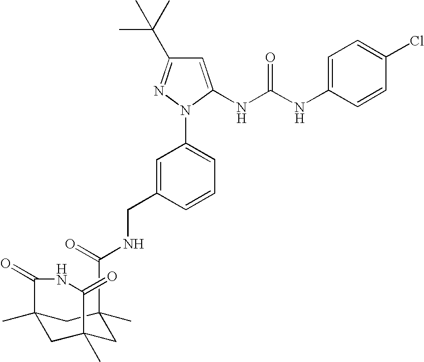

### Example 10

To title compound, was synthesized in a manner analogous to Example 9 utilizing Example J to yield 1,5,7-trimethyl-2,4-dioxo-3-aza-bicyclo[3.3.1]nonane-7-carboxylic acid 3-{3-t-butyl-5-[3-(4-chloro-phenyl)-ureido]-pyrazol-1-yl}benzylamide. ¹H NMR (CDCl₃): *8.48 (s, 1H), 7.78 (s, 1H), 7.75 (d, J=8.0 Hz, 1H), 7.69 (s, 1H), 7.53 (t, J=8.0 Hz, 1H), 7.48 (d, J=8.8 Hz, 2H), 7.26 (m, 3H), 6.62 (s, 1H), 6.35 (t, J=6.0 Hz, 1H), 5.69 (brs, 1H), 4.26 (d, J=6.0 Hz, 2H), 2.48 (d, J=14.0 Hz, 2H), 1.87 (d, J=13.6 Hz, 1H), 1.35 (s, 9H), 1.25 (m, 6H), 1.15 (s, 6H); MS (ESI) m/z: 619 (M+H⁺).

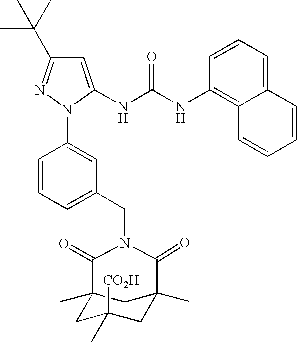

### Example 11

A mixture of Example I (41 mg, 0.1 mmol), Kemp acid anhydride (24 mg, 0.1 mmol) and Et₃N (100 mg, 1 mmol) in anhydrous CH₂Cl₂ (2 mL) were stirred overnight at RT, and concentrated in vacuo. Anhydrous benzene (20 mL) was added to the residue, the mixture was refluxed for 3 h, concentrated in vacuo and purified by preparative HPLC to yield 3-{3-[3-t-butyl-5-(3-naphthalen-1-yl-ureido)-pyrazol-1-yl]-benzyl}-1,5-di-methyl-2,4-dioxo-3-aza-bicyclo[3.3.1]nonane-7-carboxylic acid (8.8 mg, 14%). ¹H NMR (CD₃OD): *7.3-7.4 (m, 2H), 7.20 (m, 2H), 7.4-7.6 (m, 7H), 6.50 (m, 1H), 4.80 (s, 2H), 2.60 (d, J=14 Hz, 2H), 1.90 (m, 1H), 1.40 (m, 1H), 1.30 (m, 2H), 1.20 (s, 3H), 1.15 (s, 6H); MS (ESI) m/z: 636 (M+H⁺).

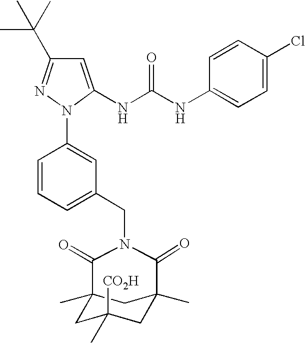

### Example 12

The title compound was synthesized in a manner analogous to Example 11 utilizing Example J to yield 3-{3-[3-t-butyl-5-(3-naphthalen-1-yl-ureido)-pyrazol-1-yl]-benzyl}-1,5-dimethyl-2,4-dioxo-3-aza-bicyclo[3.3.1]nonane-7-carboxylic acid. ¹H NMR (CD₃OD): *7.2-7.5 (m, 7H), 6.40 (s 1H), 4.70 (s, 2H), 2.60 (d, J=14 Hz, 2H), 1.90 (m, 1H), 1.50 (m, 1H), 1.45 (s, 9H), 1.30 (m, 2H), 1.21 (s, 3H), 1.18 (s, 6H); MS (ESI) m/z: 620 (M+H⁺).

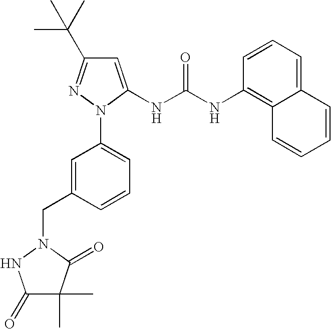

### Example 13

The title compound was synthesized in a manner analogous to Example 1 utilizing Example E and 4,4-dimethyl-3,5-dioxo-pyrazolidine to yield 1-(3-t-butyl-1-{3-[(4,4-dimethyl-3,5-dioxopyrazolidin-1-yl)methyl]phenyl}-1H-pyrazol-5-yl)-3-(naphthalen-1-yl)urea. ¹H NMR (CD₃OD): *7.88-7.86 (m, 1H), 7.71-7.68 (m, 2H), 7.58 (m, 2H), 7.60-7.42 (m, 5H), 6.49 (s, 1H), 4.85 (s, 1H), 1.34 (s, 9H), 1.27 (s, 6H); MS (ESI) m/z: 525 (M+H⁺).

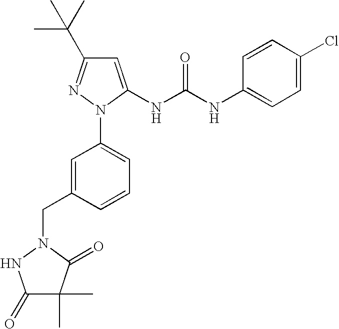

### Example 14

The title compound was synthesized in a manner analogous to Example 1 utilizing Example G and 4,4-dimethyl-3,5-dioxo-pyrazolidine to yield 1-(3-t-butyl-1-{3-[(4,4-dimethyl-3,5-dioxopyrazolidin-1-yl)methyl]phenyl}-1H-pyrazol-5-yl)-3-(4-chlorophenyl)urea. ¹H NMR (CD₃OD): *7.60-7.20 (m, 8H), 6.43 (s, 1H), 4.70 (s, 1H), 1.34 (s, 9H), 1.26 (s, 6H); MS (ESI) m/z: 509,511 (M+H⁺).

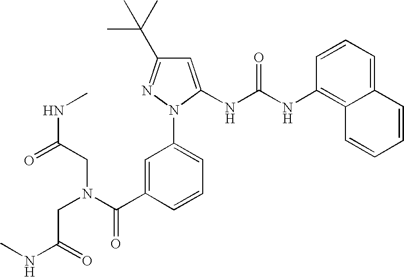

### Example 15

### Example B was saponified with 2N LiOH in MeOH, and to the resulting acid (64.2 mg, 0.15 mmol) were added HOBt (30 mg, 0.225 mmol), Example K (24 mg, 0.15 mmol) and 4-methylmorpholine (60 mg, 0.60 mmol 4.0 equiv), DMF (3 mL) and EDCI (43 mg, 0.225 mmol). The reaction mixture was stirred at RT overnight and poured into H₂O (3 mL), and a white precipitate collected and further purified by preparative HPLC to yield 1-[1-(3-{bis[(methylcarbamoyl)methyl]carbamoyl}phenyl)-3-t-butyl-1H-pyrazol-5-yl]-3-(naphthalen-1-yl)urea (40 mg). ¹H NMR (CDCl₃): *8.45 (brs, 1H), 8.10 (d, J=7.6 Hz, 1H), 7.86-7.80 (m, 2H), 7.63-7.56 (m, 2H), 7.52 (s, 1H), 7.47-7.38 (m, 3H), 7.36-7.34 (m, 1H), 7.26 (s, 1H), 7.19-7.17 (m, 2H), 6.60 (s, 1H), 3.98 (s, 2H), 3.81 (s, 3H), 2.87 (s, 3H), 2.63 (s, 3H), 1.34 (s, 9H); MS (ESI) m/z: 570 (M+H⁺).

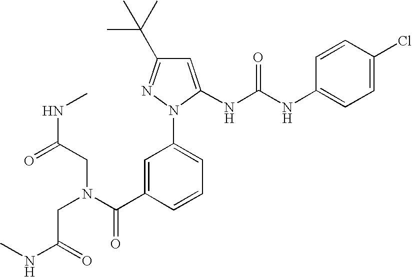

### Example 16

The title compound was synthesized in a manner analogous to Example 15 utilizing Example C (37 mg) and Example K to yield 1-[1-(3-{bis[(methylcarbamoyl)methyl]carbamoyl}phenyl)-3-t-butyl-1H-pyrazol-5-yl]-3-(4-chlorophenyl)urea. ¹H NMR (CD₃OD): *8.58 (brs, 1H), 8.39 (brs, 1H), 7.64-7.62 (m, 3H), 7.53-7.51 (m, 1H), 7.38 (d, J=9.2 Hz, 2H), 7.25 (d, J=8.8 Hz, 2H), 6.44 (s, 1H), 4.17 (s, 2H), 4.11 (s, 2H), 2.79 (s, 3H), 2.69 (s, 3H), 1.34-1.28 (m, 12H); MS (ESI) m/z: 554 (M+H⁺).

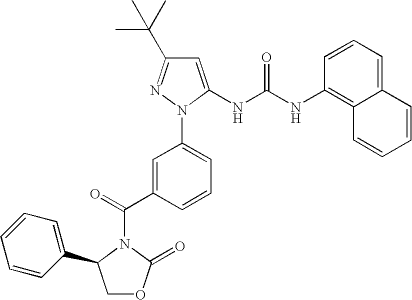

### Example 17

### Example B was saponified with 2N LiOH in MeOH, and to the resulting acid (0.642 g, 1.5 mmol) in dry THF (25 mL) at −78° C. were added freshly distilled triethylamine (0.202 g, 2.0 mmol) and pivaloyl chloride (0.216 g, 1.80 mmol) with vigorous stirring. After stirring at −78° C. for 15 min and at 0° C. for 45 min, the mixture was again cooled to −78° C. and then transferred into the THF solution of lithium salt of D-4-phenyl-oxazolidin-2-one [*: The lithium salt of the oxazolidinone regeant was previously prepared by the slow addition of n-BuLi (2.50M in hexane, 1.20 mL, 3.0 mmol) into THF solution of D-4-phenyl-oxazoldin-2-one at −78° C.]. The reaction solution was stirred at −78° C. for 2 h and RT overnight, and then quenched with aq. ammonium chloride and extracted with dichloromethane (100 mL). The combined organic layers were dried (Na₂SO₄) and concentrated in vacuo. The residue was purified by preparative HPLC to yield D-1-(5-t-butyl-2-[3-(2-oxo-4-phenyl-oxazolidinyl-3-carbonyl)phenyl]-2H-pyrazol-3-yl)-3-(naphthalen-1-yl)urea (207 mg, 24%). ¹H NMR (CDCl₃): *8.14-8.09 (m, 2H), 8.06 (s, 1H), 7.86-7.81 (m, 4H), 7.79 (s, 1H), 7.68-7.61 (m, 2H), 7.51-7.40 (m, 9H), 6.75 (s, 1H), 5.80 (t, J=9.2, 7.6 Hz, 1H), 4.89 (t, J=9.2 Hz, 1H), 4.42 (dd, J=9.2, 7.6 Hz, 1H), 1.37 (s, 9H); MS (ESI) m/z: 574 (M+H⁺).

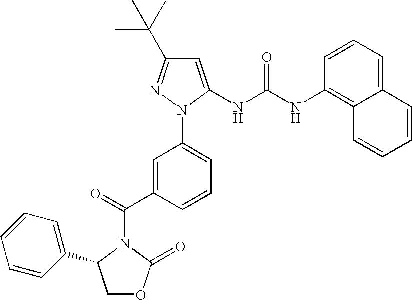

### Example 18

The title compound was synthesized in a manner analogous to Example 17 utilizing Example B and L-4-phenyl-oxazolidin-2-one to yield L-1-{5-t-butyl-2-[3-(2-oxo-4-phenyl-oxazolidinyl-3-carbonyl)phenyl]-2H-pyrazol-3-yl}-3-(naphthalen-1-yl)urea ¹H NMR (CDCl₃): *8.14-8.09 (m, 2H), 8.06 (s, 1H), 7.86-7.81 (m, 4H), 7.79 (s, 1H), 7.68-7.61 (m, 2H), 7.51-7.40 (m, 9H), 6.75 (s, 1H), 5.80 (t, J=9.2, 7.6 Hz, 1H), 4.89 (t, J=9.2 Hz, 1H), 4.42 (dd, J=9.2, 7.6 Hz, 1H), 1.37 (s, 9H); MS (ESI) m/z: 574 (M+H⁺)

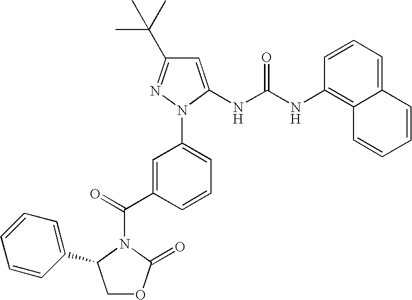

### Example 19

The title compound was synthesized in a manner analogous to Example 17 utilizing Example C and D-4-phenyl-oxazolidin-2-one to yield D-1-{5-t-butyl-2-[3-(2-oxo-4-phenyl-oxazolidinyl-3-carbonyl)phenyl]-2H-pyrazol-3-yl}-3-(4-chlorophenyl)urea. ¹H NMR (CDCl₃): *7.91 (s, 1H), 7.85 (d, J=8.0 Hz, 1H), 7.79 (d, J=7.6 Hz, 1H), 7.71 (m, 1H), 7.65 (m, 1H), 7.49-7.40 (m, 8H), 7.26-7.24 (m, 2H), 6.68 (s, 1H), 5.77 (dd, J=8.8, 8.0 Hz, 1H), 4.96 (t, 8.8 Hz, 1H), 4.44 (dd, J=8.8, 8.0 Hz, 1H), 1.36 (s, 9H); MS (ESI) m/z: 558 (M+H⁺)

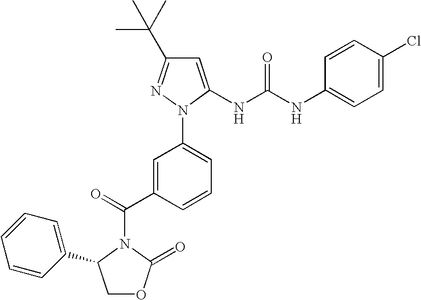

### Example 20

The title compound was synthesized in a manner analogous to Example 17 utilizing Example C and L-4-phenyl-oxazolidin-2-one to yield L-1-{5-t-butyl-2-[3-(2-oxo-4-phenyl-oxazolidinyl-3-carbonyl)phenyl]-2H-pyrazol-3-yl}-3-(4-chlorophenyl)urea. ¹H NMR (CDCl₃): *7.91 (s, 1H), 7.85 (d, J=8.0 Hz, 1H), 7.79 (d, J=7.6 Hz, 1H), 7.71 (m, 1H), 7.65 (m, 1H), 7.49-7.40 (m, 8H), 7.26-7.24 (m, 2H), 6.68 (s, 1H), 5.77 (dd, J=8.8, 8.0 Hz, 1H), 4.96 (t, 8.8 Hz, 1H), 4.44 (dd, J=8.8, 8.0 Hz, 1H), 1.36 (s, 9H); MS (ESI) m/z: 558 (M+Ht)

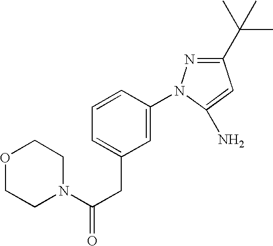

### Example L

To a stirred suspension of (3-nitro-phenyl)-acetic acid (2 g) in CH₂Cl₂ (40 ml, with a catalytic amount of DMF) at 0° C. under N₂ was added oxalyl chloride (1.1 ml) drop wise. The reaction mixture was stirred for 40 min morpholine (2.5 g) was added. After stirring for 20 min, the reaction mixture was filtered. The filtrate was concentrated in vacuo to yield 1-morpholin-4-yl-2-(3-nitro-phenyl)-ethanone as a solid (2 g). A mixture of 1-morpholin-4-yl-2-(3-nitro-phenyl)-ethanone (2 g) and 10% Pd on activated carbon (0.2 g) in ethanol (30 ml) was hydrogenated at 30 psi for 3 h and filtered over Celite. Removal of the volatiles in vacuo provided 2-(3-amino-phenyl)-1-morpholin-4-yl-ethanone (1.7 g). A solution of 2-(3-amino-phenyl)-1-morpholin-4-yl-ethanone (1.7 g, 7.7 mmol) was dissolved in 6 N HCl (15 ml), cooled to 0° C., and vigorously stirred. Sodium nitrite (0.54 g) in water (8 ml) was added. After 30 min, tin (II) chloride dihydrate (10 g) in 6 N HCl (30 ml) was added. The reaction mixture was stirred at 0° C. for 3 h. The pH was adjusted to pH 14 with solid potassium hydroxide and extracted with EtOAc. The combined organic extracts were concentrated in vacuo provided 2-(3-hydrazin-phenyl)-1-morpholin-4-yl-ethanone (1.5 g). 2-(3-Hydrazinophenyl)-1-morpholin-4-yl-ethanone (3 g) and 4,4-dimethyl-3-oxopentanenitrile (1.9 g, 15 mmol) in ethanol (60 ml) and 6 N HCl (1 ml) were refluxed for 1 h and cooled to RT. The reaction mixture was neutralized by adding solid sodium hydrogen carbonate. The slurry was filtered and removal of the volatiles in vacuo provided a residue that was extracted with ethyl acetate. The volatiles were removed in vacuo to provide 2-[3-(3-t-butyl-5-amino-1H-pyrazol-1-yl)phenyl]-1-morpholinoethanone (4 g), which was used without further purification.

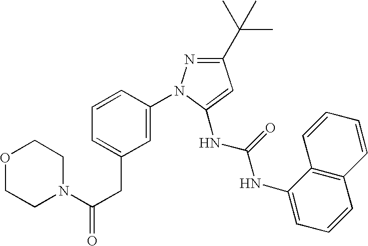

### Example 21

A mixture of Example L (0.2 g, 0.58 mmol) and 1-naphthylisocyanate (0.10 g, 0.6 mmol) in dry CH₂Cl₂ (4 ml) was stirred at RT under N₂ for 18 h. The solvent was removed in vacuo and the crude product was purified by column chromatography using ethyl acetate/hexane/CH₂Cl₂ (3/1/0.7) as the eluent (0.11 g, off-white solid) to yield 1-{3-t-butyl-1-[3-(2-morpholino-2-oxoethyl)phenyl]-1H-pyrazol-5-yl}-3-(naphthalene-1-yl)urea. mp: 194-196; ¹H NMR (200 MHz, DMSO-d₆): δ 9.07 (1H, s), 8.45 (s, 1H), 8.06-7.93 (m, 3H), 7.69-7.44 (m, 7H), 7.33-7.29 (d, 6.9 Hz, 1H), 6.44 (s, 1H), 3.85 (m, 2H), 3.54-3.45 (m, 8H), 1.31 (s, 9H); MS:

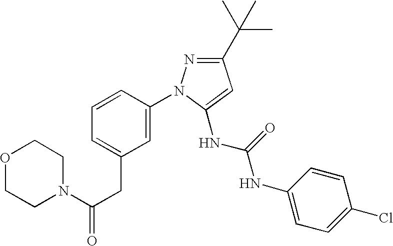

### Example 22

The title compound was synthesized in a manner analogous to Example 21 utilizing Example L (0.2 g, 0.58 mmol) and 4-chlorophenylisocyanate (0.09 g, 0.6 mmol) to yield 1-{3-t-butyl-1-[3-(2-morpholino-2-oxoethyl)phenyl]-1H-pyrazol-5-yl}-3-(4-chlorophenyl)urea. mp: 100 104; ¹H NMR (200 MHz, DMSO-d₆): δ 9.16 (s, 1H), 8.45 (s, 1H), 7.52-7.30 (m, 8H), 6.38 (s, 1H), 3.83 (m, 1H), 3.53-3.46 (m, 8H), 1.30 (s, 9H); MS:

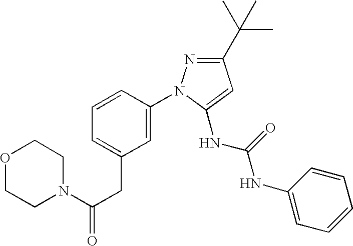

### Example 23

The title compound is synthesized in a manner analogous to Example 21 utilizing Example L (0.2 g, 0.58 mmol) and phenylisocyanate (0.09 g, 0.6 mmol) to yield 1-(3-t-butyl-1-[3-(2-morpholino-2-oxoethyl)phenyl]-1H-pyrazol-5-yl)-3-phenylurea.

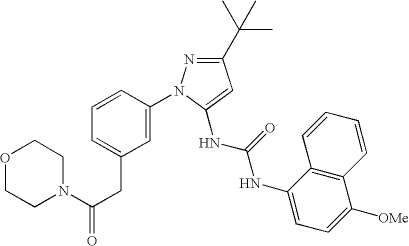

### Example 24

The title compound is synthesized in a manner analogous to Example 21 utilizing Example L (0.2 g, 0.58 mmol) and 1-isocyanato-4-methoxy-naphthalene to yield 1-{3-t-butyl-1-[3-(2-morpholino-2-oxoethyl)phenyl]-1H-pyrazol-5-yl}-3-(1-methoxynaphthalen-4-yl)urea.

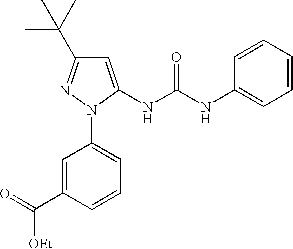

### Example M

The title compound is synthesized in a manner analogous to Example C utilizing Example A and phenylisocyanate to yield ethyl 3-(3-t-butyl-5-(3-phenylureido)-1H-pyrazol-1-yl)benzoate.

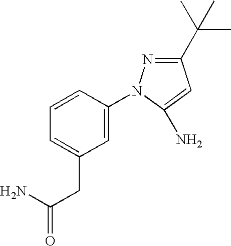

### Example N

A solution of (3-nitrophenyl)acetic acid (23 g, 127 mmol) in methanol (250 ml) and a catalytic amount of concentrated in vacuo H₂SO₄ was heated to reflux for 18 h. The reaction mixture was concentrated in vacuo to a yellow oil. This was dissolved in methanol (250 ml) and stirred for 18 h in an ice bath, whereupon a slow flow of ammonia was charged into the solution. The volatiles were removed in vacuo. The residue was washed with diethyl ether and dried to afford 2-(3-nitrophenyl)acetamide (14 g, off-white solid). ¹H NMR (CDCl₃): δ 8.1 (s, 1H), 8.0 (d, 1H), 7.7 (d, 1H), 7.5 (m, 1H), 7.1 (bd s, 1H), 6.2 (brs, 1H), 3.6 (s, 2H).

The crude material from the previous reaction (8 g) and 10% Pd on activated carbon (1 g) in ethanol (100 ml) was hydrogenated at 30 psi for 18 h and filtered over Celite. Removal of the volatiles in vacuo provided 2-(3-aminophenyl)acetamide (5.7 g). A solution of this material (7 g, 46.7 mmol) was dissolved in 6 N HCl (100 ml), cooled to 0° C., and vigorously stirred. Sodium nitrite (3.22 g, 46.7 mmol) in water (50 ml) was added. After 30 min, tin (II) chloride dihydrate (26 g) in 6 N HCl (100 ml) was added. The reaction mixture was stirred at 0° C. for 3 h. The pH was adjusted to pH 14 with 50% aqueous NaOH solution and extracted with ethyl acetate. The combined organic extracts were concentrated in vacuo provided 2-(3-hydrazinophenyl)acetamide.

The crude material from the previous reaction (ca. 15 mmol) and 4,4-dimethyl-3-oxopentanenitrile (1.85 g, 15 mmol) in ethanol (60 ml) and 6 N HCl (1.5 ml) was refluxed for 1 h and cooled to RT. The reaction mixture was neutralized by adding solid sodium hydrogen carbonate. The slurry was filtered and removal of the volatiles in vacuo provided a residue, which was extracted with ethyl acetate. The solvent was removed in vacuo to provide 2-[3-(3-t-butyl-5-amino-1H-pyrazol-1-yl)phenyl]acetamide as a white solid (3.2 g), which was used without further purification.

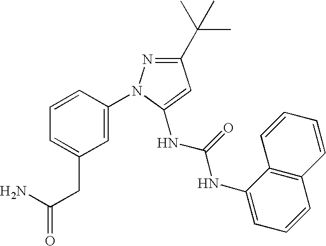

### Example 25

A mixture of Example N (2 g, 0.73 mmol) and 1-naphthylisocyanate (0.124 g, 0.73 mmol) in dry CH₂Cl₂ (4 ml) was stirred at RT under N₂ for 18 h. The solvent was removed in vacuo and the crude product was washed with ethyl acetate (8 ml) and dried in vacuo to yield 1-{3-t-butyl-1-[3-(carbamoylmethyl)phenyl)-1H-pyrazol-5-yl}-3-(naphthalene-1-yl)urea as a white solid (0.22 g). mp: 230 (dec.); ¹H NMR (200 MHz, DMSO-d₆): δ 9.12 (s, 1H), 8.92 (s, 1H), 8.32-8.08 (m, 3H), 7.94-7.44 (m, 8H), 6.44 (s, 1H), 3.51 (s, 2H), 1.31 (s, 9H); MS:

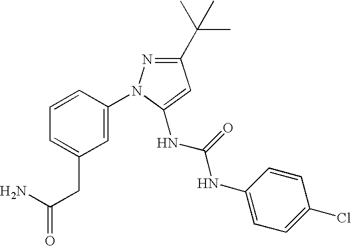

### Example 26

The title compound was synthesized in a manner analogous to Example 23 utilizing Example N (0.2 g, 0.73 mmol) and 4-chlorophenylisocyanate (0.112 g, 0.73 mmol) to yield 1-{3-t-butyl-1-[3-(carbamoylmethyl)phenyl)-1H-pyrazol-5-yl}-3-(4-chlorophenyl)urea as a white solid (0.28 g). mp: 222 224. (dec.); ¹H NMR (200 MHz, DMSO-d₆); o 9.15 (s, 1H), 8.46 (s, 1H), 7.55-7.31 (m, 8H), 6.39 (s, 1H), 3.48 (s, 2H), 1.30 (s, 9H); MS:

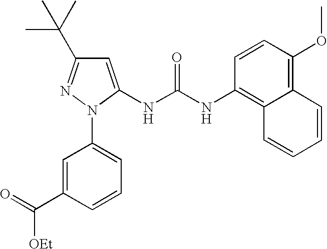

### Example O

The title compound is synthesized in a manner analogous to Example C utilizing Example A and 1-isocyanato-4-methoxy-naphthaleneto yield ethyl 3-(3-t-butyl-5-(3-(1-methoxynaphthalen-4-yl)ureido)-1H-pyrazol-1-yl)benzoate.

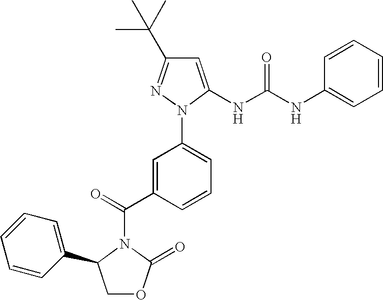

### Example 27

The title compound is synthesized in a manner analogous to Example 17 utilizing Example M and D-4-phenyl-oxazolidin-2-one to yield D-1-{(5-t-butyl-2-[3-(2-oxo-4-phenyl-oxazolidinyl-3-carbonyl)phenyl]-2H-pyrazol-3-yl}-3-phenylurea.

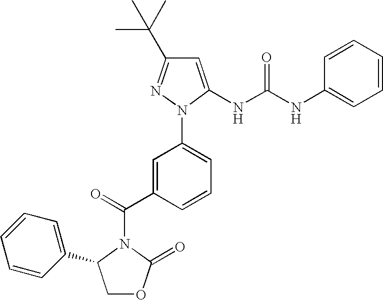

### Example 28

The title compound is synthesized in a manner analogous to Example 17 utilizing Example M and L-4-phenyl-oxazolidin-2-one to yield L-1-{5-t-butyl-2-[3-(2-oxo-4-phenyl-oxazolidinyl-3-carbonyl)phenyl]-2H-pyrazol-3-yl}-3-phenylurea.

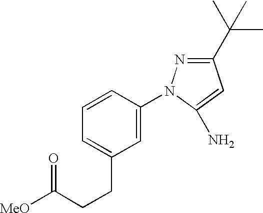

### Example P

A mixture of 3-(3-amino-phenyl)-acrylic acid methyl ester (6 g and 10% Pd on activated carbon (1 g) in ethanol (50 ml) was hydrogenated at 30 psi for 18 h and filtered over Celite. Removal of the volatiles in vacuo provided 3-(3-amino-phenyl)propionic acid methyl ester (6 g).

A vigorously stirred solution of the crude material from the previous reaction (5.7 g, 31.8 mmol) dissolved in 6 N HCl (35 ml) was cooled to 0° C., and sodium nitrite (2.2 g) in water (20 ml) was added. After 1 h, tin (II) chloride dihydrate (18 g) in 6 N HCl (35 ml) was added. And the mixture was stirred at 0° C. for 3 h. The pH was adjusted to pH 14 with solid KOH and extracted with EtOAc. The combined organic extracts were concentrated in vacuo provided methyl 3-(3-hydrazino-phenyl)propionate (1.7 g).

A stirred solution of the crude material from the previous reaction (1.7 g, 8.8 mmol) and 4,4-dimethyl-3-oxopentanenitrile (1.2 g, 9.7 mmol) in ethanol (30 ml) and 6 N HCl (2 ml) was refluxed for 18 h and cooled to RT. The volatiles were removed in vacuo and the residue dissolved in EtOAc and washed with 1 N aqueous NaOH. The organic layer was dried (Na₂SO₄) and concentrated in vacuo and the residue was purified by column chromatography using 30% ethyl acetate in hexane as the eluent to provide methyl 3-[3-(3-t-butyl-5-amino-1H-pyrazol-1-yl)phenyl]propionate (3.2 g), which was used without further purification

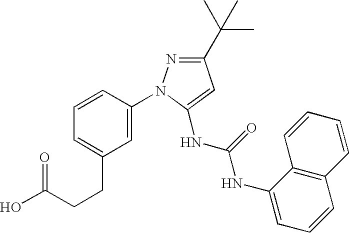

### Example 29

A mixture of Example P (0.35 g, 1.1 mmol) and 1-naphthylisocyanate (0.19 g, 1.05 mmol) in dry CH₂Cl₂ (5 ml) was stirred at RT under N₂ for 20 h. The solvent was removed in vacuo and the residue was stirred in a solution of THF (3 ml)/MeOH (2 ml)/water (1.5 ml) containing lithium hydroxide (0.1 g) for 3 h at RT, and subsequently diluted with EtOAc and dilute citric acid solution. The organic layer was dried (Na₂SO₄), and the volatiles removed in vacuo. The residue was purified by column chromatography using 3% methanol in CH₂Cl₂ as the eluent to yield 3-(3-{3-t-butyl-5-[3-(naphthalen-1-yl)ureido]-1H-pyrazol-1-yl)phenylpropionic acid (0.22 g, brownish solid). mp: 105-107; ¹H NMR (200 MHz, CDCl₃): δ 7.87-7.36 (m, 10H), 7.18-7.16 (m, 1H), 6.52 (s, 1H), 2.93 (t, J=6.9 Hz, 2H), 2.65 (t, J=7.1 Hz, 2H), 1.37 (s, 9H); MS

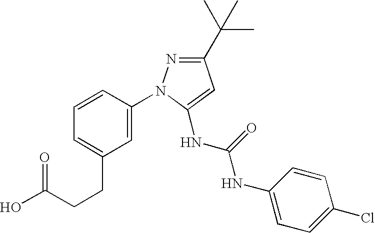

### Example 30

The title compound was synthesized in a manner analogous to Example 29 utilizing Example P (0.30 g, 0.95 mmol) and 4-chlorophenylisocyanate (0.146 g, 0.95 mmol) to yield 3-(3-{3-t-butyl-5-[3-(4-chlorophenyl)ureido]-1H-pyrazol-1-yl)phenyl)propionic acid (0.05 g, white solid). mp: δ 87; ¹H NMR (200 MHz, CDCl₃): δ 8.21 (s, 1H), 7.44-7.14 (m, 7H), 6.98 (s, 1H), 6.55 (s, 1H), 2.98 (t, J=5.2 Hz, 2H), 2.66 (t, J=5.6 Hz, 2H), 1.40 (s, 9H); MS

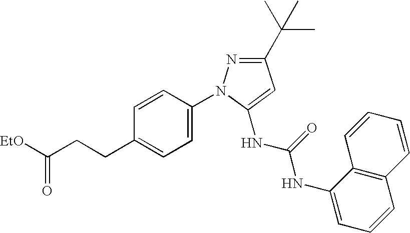

### Example Q

A mixture of ethyl 3-(4-aminophenyl)acrylate (1.5 g) and 10% Pd on activated carbon (0.3 g) in ethanol (20 ml) was hydrogenated at 30 psi for 18 h and filtered over Celite. Removal of the volatiles in vacuo provided ethyl 3-(4-aminophenyl)propionate (1.5 g).

A solution of the crude material from the previous reaction (1.5 g, 8.4 mmol) was dissolved in 6 N HCl (9 ml), cooled to 0° C., and vigorously stirred. Sodium nitrite (0.58 g) in water (7 ml) was added. After 1 h, tin (II) chloride dihydrate (5 g) in 6 N HCl (10 ml) was added. The reaction mixture was stirred at 0° C. for 3 h. The pH was adjusted to pH 14 with solid KOH and extracted with EtOAc. The combined organic extracts were concentrated in vacuo provided ethyl 3-(4-hydrazino-phenyl)-propionate (1 g).

The crude material from the previous reaction (1 g, 8.8 mmol) and 4,4-dimethyl-3-oxopentanenitrile (0.7 g) in ethanol (8 ml) and 6 N HCl (1 ml) was refluxed for 18 h and cooled to RT. The volatiles were removed in vacuo. The residue was dissolved in ethyl acetate and washed with 1 N aqueous sodium hydroxide solution. The organic layer was dried (Na₂SO₄) and concentrated in vacuo. The residue was purified by column chromatography using 0.7% methanol in CH₂Cl₂ as the eluent to provide ethyl 3-{4-[3-t-butyl-5-(3-(naphthalene-1-yl)ureido]-1H-pyrazol-1-yl}phenyl)propanoate (0.57 g).

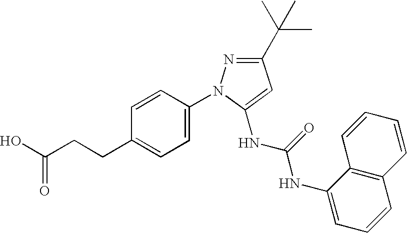

### Example 31

A mixture of Example Q (0.25 g, 0.8 mmol) and 1-naphthylisocyanate (0.13 g, 0.8 mmol) in dry CH₂Cl₂ (5 ml) was stirred at RT under N₂ for 20 h. The solvent was removed in vacuo and the residue was stirred in a solution of THF (3 ml)/MeOH (2 ml)/water (1.5 ml) containing lithium hydroxide (0.1 g) for 3 h at RT and diluted with EtOAc and diluted citric acid solution. The organic layer was dried (Na₂SO₄), and the volatiles removed in vacuo. The residue was purified by column chromatography using 4% methanol in CH₂Cl₂ as the eluent to yield 3-{4-[3-t-butyl-5-(3-(naphthalene-1-yl)ureido]-1H-pyrazol-1-yl}phenyl)propanonic acid (0.18 g, off-white solid). mp: 120 122; ¹H NMR (200 MHz, CDCl₃): δ 7.89-7.06 (m, 11H), 6.5 (s, 1H), 2.89 (m, 2H), 2.61 (m, 2H), 1.37 (s, 9H); MS

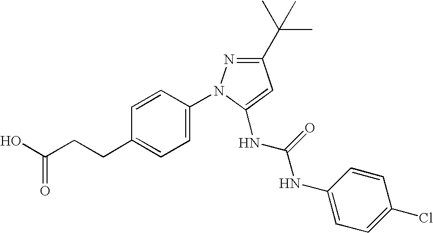

### Example 32

The title compound was synthesized in a manner analogous to Example 31 utilizing Example Q (0.16 g, 0.5 mmol) and 4-chlorophenylisocyanate (0.077 g, 0.5 mmol) to yield 3-{4-[3-t-butyl-5-(3-(4-chlorophenyl)ureido]-1H-pyrazol-1-yl}phenyl)propanonic acid (0.16 g, off-white solid). mp: 112-114 ¹H NMR (200 MHz, CDCl₃): δ 8.16 (s, 1H), 7.56 (s, 1H), 7.21 (s, 2H), 7.09 (s, 2H), 6.42 (s, 1H), 2.80 (m, 2H), 2.56 (m, 2H), 1.32 (s, 9H); MS

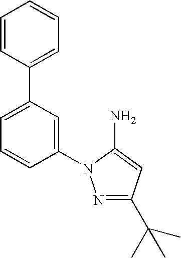

### Example R

A 250 mL pressure vessel (ACE Glass Teflon screw cap) was charged with 3-nitrobiphenyl (20 g, 0.10 mol) dissolved in THF (−100 mL) and 10% Pd/C (3 g). The reaction vessel was charged with H₂ (g) and purged three times. The reaction was charged with 40 psi H₂ (g) and placed on a Parr shaker hydrogenation apparatus and allowed to shake overnight at RT. HPLC showed that the reaction was complete thus the reaction mixture was filtered through a bed of Celite and evaporated to yield the amine: 16.7 g (98% yield)

In a 250 mL Erlenmeyer flask with a magnetic stir bar, the crude material from the previous reaction (4.40 g, 0.026 mol) was added to 6 N HCl (40 mL) and cooled with an ice bath to −0° C. A solution of NaNO₂ (2.11 g, 0.0306 mol, 1.18 eq.) in water (5 mL) was added drop wise. After 30 min, SnCl₂2H₂O (52.0 g, 0.23 mol, 8.86 eq.) in 6N HCl (100 mL) was added and the reaction mixture was allowed to stir for 3 h, then subsequently transferred to a 500 mL round bottom flask. To this, 4,4-dimethyl-3-oxopentanenitrile (3.25 g, 0.026 mol) and EtOH (100 ml) were added and the mixture refluxed for 4 h, concentrated in vacuo and the residue extracted with EtOAc (2×100 mL). The residue was purified by column chromatograph using hexane/EtOAc/Et-N (8:2:0.2) to yield 0.53 g of Example R. ¹H NMR (CDCl₃): δ 7.5 (m, 18H), 5.8 (s, 1H), 1.3 (s, 9H).

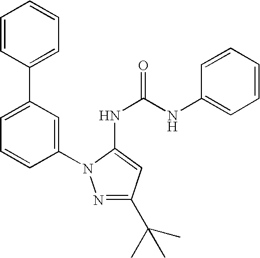

### Example 33

In a dry vial with a magnetic stir bar, Example R (0.145 g; 0.50 mmol) was dissolved in 2 mL CH₂Cl₂ (anhydrous) followed by the addition of phenylisocyanate (0.0544 mL; 0.50 mmol; 1 eq.). The reaction was kept under argon and stirred for 17 h. Evaporation of solvent gave a crystalline mass that was triturated with hexane/EtOAc (4:1) and filtered to yield 1-(3-t-butyl-1-(3-phenylphenyl)-1H-pyrazol-5-yl)-3-phenylurea (0.185 g, 90%). HPLC purity: 96%; mp: 80 84; ¹H NMR (CDCl₃): δ 7.3 (m, 16H), 6.3 (s, 1H), 1.4 (s, 9H).

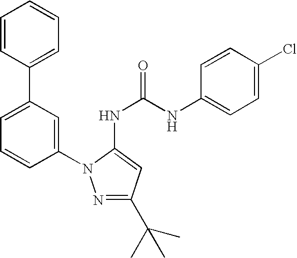

### Example 34

The title compound was synthesized in a manner analogous to Example 33 utilizing Example R (0.145 g; 0.50 mmol) and p-chlorophenylisocyanate (0.0768 g, 0.50 mmol, 1 eq.) to yield 1-(3-t-butyl-1-(3-phenylphenyl)-1H-pyrazol-5-yl)-3-(4-chlorophenyl)urea (0.205 g, 92%). HPLC purity: 96.5%; mp: 134 136; ¹H NMR (CDCl₃): δ 7.5 (m, 14H), 7.0 (s, 1H), 6.6 (s, 1H), 6.4 (s, 1H), 1.4 (s, 9H).

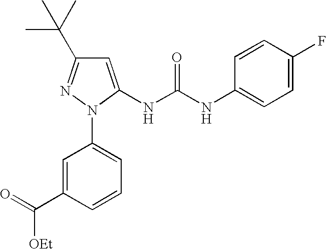

### Example S

The title compound is synthesized in a manner analogous to Example C utilizing Example A and 4-fluorophenyl isocyanate yield ethyl 3-(3-t-butyl-5-(3-(4-fluorophenyl)ureido)-1H-pyrazol-1-yl)benzoate.

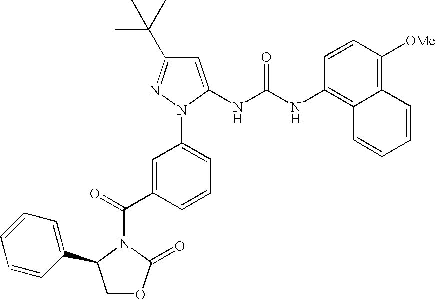

### Example 35

The title compound is synthesized in a manner analogous to Example 17 utilizing Example M and D-4-phenyl-oxazolidin-2-one to yield D-1-{5-t-butyl-2-[3-(2-oxo-4-phenyl-oxazolidinyl-3-carbonyl)phenyl]-2H-pyrazol-3-yl}-3-(naphthalen-1-yl)urea.

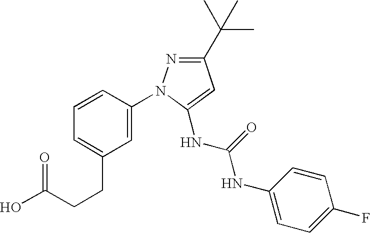

### Example 36

The title compound is synthesized in a manner analogous to Example 29 utilizing Example P (0.30 g, 0.95 mmol) and 4-fluorophenylisocyanate (0.146 g, 0.95 mmol) to yield 3-(3-(3-t-butyl-5-(3-(4-fluorophenyl)ureido)-1H-pyrazol-1-yl)phenyl)propanoic acid.

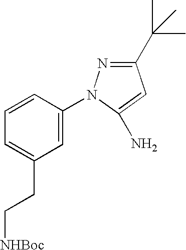

### Example T

To a stirred solution of Example N (2 g, 7.35 mmol) in THF (6 ml) was added borane-methylsulfide (18 mmol). The mixture was heated to reflux for 90 min and cooled to RT, after which 6 N HCl was added and heated to reflux for 10 min. The mixture was basified with NaOH and extracted with EtOAc. The organic layer was dried (Na₂SO₄) filtered and concentrated in vacuo to yield 3-t-butyl-1-[3-(2-aminoethyl)phenyl]-1H-pyrazol-5 amine (0.9 g).

A mixture of the crude material from the previous reaction (0.8 g, 3.1 mmol) and di-t-butylcarbonate (0.7 g, 3.5 mmol) and catalytically amount of DMAP in dry CH₂Cl₂ (5 ml) was stirred at RT under N₂ for 18 h. The reaction mixture was concentrated in vacuo and the residue was purified by column chromatography using 1% methanol in CH₂Cl₂ as the eluent to yield t-butyl 3-(3-t-butyl-5-amino-1H-pyrazol-1-yl)phenylcarbamate (0.5 g).

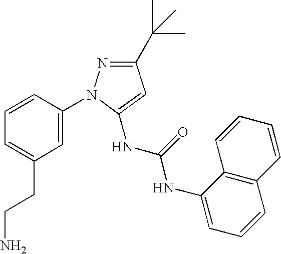

### Example 37

A mixture of Example T (0.26 g, 0.73 mmol) and 1-naphthylisocyanate (0.123 g, 0.73 mmol) in dry CH₂Cl₂ (5 ml) was stirred at RT under N₂ for 48 h. The solvent was removed in vacuo and the residue was purified by column chromatography using 1% methanol in CH₂Cl₂ as the eluent (0.15 g, off-white solid). The solid was then treated with TFA (0.2 ml) for 5 min and diluted with EtOAc. The organic layer was washed with saturated NaHCO₃ solution and brine, dried (Na₂SO₄), filtered and concentrated in vacuo to yield 1-{3-t-butyl-1-[3-(2-Aminoethyl)phenyl]-1H-pyrazol-5-yl}-3-(naphthalen-1-yl)urea as a solid (80 mg). mp: 110-112; 2H NMR (200 MHz, DMSO-d₆): δ 9.09 (s, 1H), 8.90 (s, 1H), 8.01-7.34 (m, 11H), 6.43 (s, 1H), 3.11 (m, 2H), 2.96 (m, 2H), 1.29 (s, 9H); MS

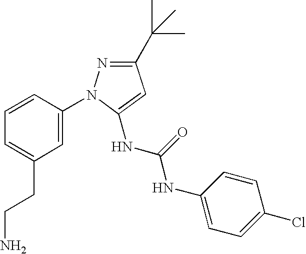

### Example 38

The title compound was synthesized in a manner analogous to Example 37 utilizing Example T (0.15 g, 0.42 mmol) and 4-chlorophenylisocyanate (0.065 g, 0.42 mmol) to yield 1-{3-t-butyl-1-[3-(2-Aminoethyl)phenyl]-1H-pyrazol-5-yl}-3-(4-chlorophenyl)urea as an off-white solid (20 mg). mp: 125-127; ¹H NMR (200 MHz, CDCl₃): δ 8.81 (s, 1H), 8.66 (s, 1H), 7.36-7.13 (m, 8H), 6.54 (s, 1H), 3.15 (brs, 2H), 2.97 (brs, 2H), 1.32 (s, 9H); MS

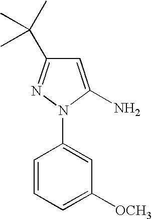

### Example U

In a 250 mL Erlenmeyer flask with a magnetic stir bar, m-anisidine (9.84 g, 0.052 mol) was added to 6 N HCl (80 mL) and cooled with an ice bath to 0° C. A solution of NaNO₂ (4.22 g, 0.0612 mol, 1.18 eq.) in water (10 mL) was added drop wise. After 30 min, SnCl₂2H₂O (104.0 g, 0.46 mol, 8.86 eq.) in 6 N HCl (200 mL) was added and the reaction mixture was allowed to stir for 3 h., and then subsequently transferred to a 1000 mL round bottom flask. To this, 4,4-dimethyl-3-oxopentanenitrile (8.00 g, 0.064 mol) and EtOH (200 mL) were added and the mixture refluxed for 4 h, concentrated in vacuo and the residue recrystallized from CH₂Cl₂ to yield 3-t-butyl-1-(3-methoxyphenyl)-1H-pyrazol-5-amine as the HCl salt (13.9 g).

The crude material from the previous reaction (4.65 g, 0.165 mol) was dissolved in 30 mL of CH₂Cl₂ with Et₃N (2.30 mL, 0.0165 mol, 1 eq.) and stirred for 30 min Extraction with water followed by drying of the organic phase with Na₂SO₄ and concentration in vacuo yielded a brown syrup that was the free base, 3-t-butyl-1-(3-methoxyphenyl)-1H-pyrazol-5-amine (3.82 g, 94.5%), which was used without further purification.

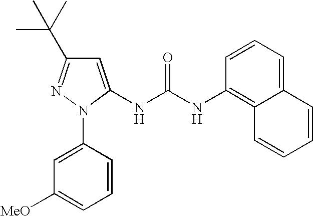

### Example 39

In a dry vial with a magnetic stir bar, Example U (2.62 g, 0.0107 mol) was dissolved in CH₂Cl₂ (5 mL, anhydrous) followed by the addition of 1-naphthylisocyanate (1.53 mL, 0.0107 mol, 1 eq.). The reaction was kept under Ar and stirred for 18 h. Evaporation of solvent followed by column chromatography with EtOAc/hexane/Et₃N (7:2:0.5) as the eluent yielded 1-[3-t-butyl-1-(3-methoxyphenyl)-1H-pyrazol-5-yl]-3-(naphthalen-1-yl)urea (3.4 g, 77%). HPLC: 97%; mp: 78-80; ¹H NMR (CDCl₃): δ 7.9-6.8 (m, 15H), 6.4 (s, 1H), 3.7 (s, 3H), 1.4 (s, 9H).

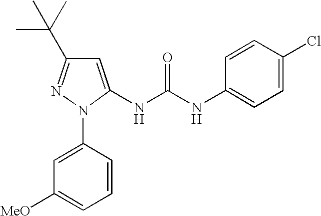

### Example 40

The title compound was synthesized in a manner analogous to Example 39 utilizing Example U (3.82 g; 0.0156 mol) and p-chlorophenylisocyanate (2.39 g, 0.0156 mol, 1 eq.), purified by trituration with hexane/EtOAc (4:1) and filtered to yield 1-[3-t-butyl-1-(3-methoxyphenyl)-1H-pyrazol-5-yl]-3-(4 chlorophenyl)urea (6.1 g, 98%). HPLC purity: 95%; mp: 158-160; ¹H NMR (CDCl₃): δ 7.7 (s, 1H); δ 7.2 6.8 (m, 8H), 6.4 (s, 1H), 3.7 (s, 3H), 1.3 (s, 9H).

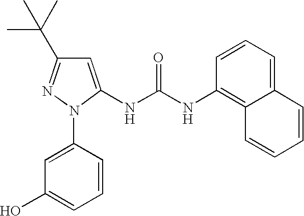

### Example 41

In a 100 ml round bottom flask equipped with a magnetic stir bar, Example 39 (2.07 g) was dissolved in CH₂Cl₂ (20 mL) and cooled to 0° C. with an ice bath. BBr₃ (1 M in CH₂Cl₂; 7.5 mL) was added slowly. The reaction mixture was allowed to warm to RT overnight. Additional BBr₃ (1 M in CH₂Cl₂, 2×1 mL, 9.5 mmol total added) was added and the reaction was quenched by the addition of MeOH. Evaporation of solvent led to a crystalline material that was chromatographed on silica gel (30 g) using CH₂Cl₂(MeOH (9.6:0.4) as the eluent to yield 1-[3-t-butyl-1-(3-hydroxyphenyl)-1H-pyrazol-5-yl]-3-(naphthalene-1-yl)urea (0.40 g, 20%). ¹H NMR (DMSO-d₆): δ 9.0 (s, 1H), 8.8 (s, 1H), 8.1-6.8 (m, 1H), 6.4 (s, 1H), 1.3 (s, 9H). MS (ESI) m/z: 401 (M+H⁺).

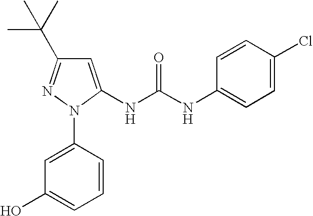

### Example 42

The title compound was synthesized in a manner analogous to Example 41 utilizing Example 40 (2.00 g, 5 mmol) that resulted in a crystalline material that was filtered and washed with MeOH to yield 1-[3-t-butyl-1-(3-hydroxyphenyl)-1H-pyrazol-5-yl]-3-(4-chlorophenyl)urea (1.14 g, 60%). HPLC purity: 96%; mp: 214-216; ¹H NMR (CDCl₃): δ 8.4 (s, 1H), 7.7 (s, 1H), 7.4-6.6 (m, 9H), 1.3 (s, 9H).

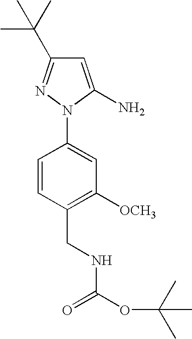

### Example V

The starting material, 1-[4-(aminomethyl)phenyl]-3-t-butyl-N-nitroso-1H-pyrazol-5-amine, was synthesized in a manner analogous to Example A utilizing 4-aminobenzamide and 4,4-dimethyl-3-oxopentanenitrile.

A 1 L four-necked round bottom flask was equipped with a stir bar, a source of dry Ar, a heating mantle, and a reflux condenser. The flask was flushed with Ar and charged with the crude material from the previous reaction (12 g, 46.5 mmol; 258.1 g/mol) and anhydrous THF (500 ml). This solution was treated cautiously with LiAlH₄ (2.65 g, 69.8 mmol) and the reaction was stirred overnight. The reaction was heated to reflux and additional LiAlH₄ was added complete (a total of 8.35 g added). The reaction was cooled to 0 and H₂O (8.4 ml), 15% NaOH (8.4 ml) and H₂O (24 ml) were added sequentially; The mixture was stirred for 2 h, the solids filtered through Celite, and washed extensively with THF, the solution was concentrated in vacuo to yield 1-(4-(aminomethyl-3-methoxy)phenyl)-3-t-butyl-1H-pyrazol-5-amine (6.8 g) as an oil.

A 40 mL vial was equipped with a stir bar, a septum, and a source of Ar. The vial was charged with the crude material from the previous reaction (2 g, 8.2 mmol, 244.17 g/mol) and CHCl₃ (15 mL) were cooled to 0 under Ar and di-t-butylcarbonate (1.9 g, 9.0 mmol) dissolved in CHCl₃ (5 mL) was added drop wise over a 2 min period. The mixture was treated with 1N KOH (2 mL), added over a 2 h period. The resulting emulsion was broken with the addition of saturated NaCl solution, the layers were separated and the aqueous phase extracted with CH₂Cl₂ (2×1.5 ml). The combined organic phases were dried over Na₂SO₄, filtered, concentrated in vacuo to yield t-butyl [4-(3-t-butyl-5-amino-1H-pyrazol-1-yl)-2-methoxybenzylcarbamate (2.23 g, 79%) as a light yellow solid. ¹H NMR (CDCl₃): δ 7.4 (m, 5H), 5.6 (s, 1H), 4.4 (d, 2H), 1.5 (s, 9H), 1.3 (s, 9H).

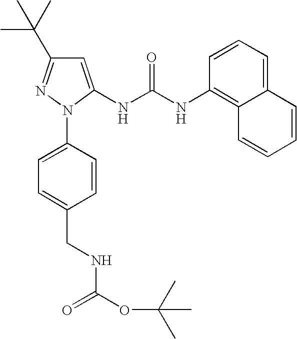

### Example 43

A 40 mL vial was equipped with a septum, a stir bar and a source of Ar, and charged with Example V (2 g, 5.81 mmol), flushed with Ar and dissolved in CHCl₃ (20 mL). The solution was treated with 2-naphthylisocyanate (984 mg, 5.81 mmol) in CHCl₃ (5 mL) and added over 1 min The reaction was stirred for 8 h, and additional 1-naphthylisocyanate (81 mg) was added and the reaction stirred overnight. The solid was filtered and washed with CH₂Cl₂ to yield t-butyl 4-[3-t-butyl-5-(3-naphthalen-1-yl)ureido)-1H-pyrazol-1-yl]benzylcarbamate (1.2 g). HPLC purity: 94.4%; ¹H NMR (DMSO-d₆): δ 9.1 (s, 1H), 8.8 (s, 1H), 8.0 (m, 3H), 7.6 (m, 9H), 6.4 (s, 1H), 4.2 (d, 2H), 1.4 (s, 9H), 1.3 (s, 9H).

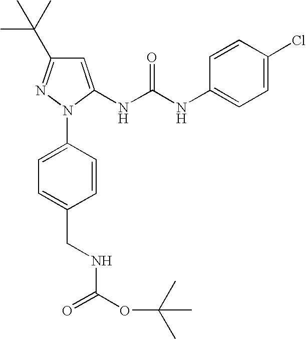

### Example 44

The title compound was synthesized in a manner analogous to Example 43 utilizing Example V (2.0 g, 5.81 mmol) and p-chlorophenylisocyanate (892 mg) to yield t-butyl 4-[3-t-butyl-5-(3-(4-chlorophenyl)ureido)-1H-pyrazol-1-yl]benzylcarbamate (1.5 g). HPLC purity: 97%; ¹H NMR (DMSO-d₆): δ 9.2 (s, 1H), 8.4 (s, 1H), 7.4 (m, 8H), 6.4 (s, 1H), 4.2 (d, 2H), 1.4 (s, 9H), 1.3 (s, 9H).

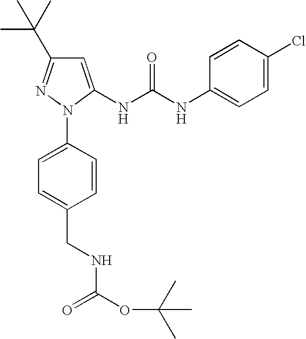

### Example 45

A 10 mL flask equipped with a stir bar was flushed with Ar and charged with Example 43 (770 mg, 1.5 mmol) and CH₂Cl₂ (1 ml) and 1:1 CH₂Cl₂:TFA (2.5 mL). After 1.5 h, reaction mixture was concentrated in vacuo, the residue was dissolved in EtOAc (15 mL), washed with saturated NaHCO₃ (10 mL) and saturated NaCl (10 mL). The organic layers was dried, filtered and concentrated in vacuo to yield 1-{3-t-butyl-1-[4-(aminomethyl)phenyl]-1H-pyrazol-5-yl}-3-(naphthalen-1-yl)urea (710 mg). ¹H NMR (DMSO-d₆): δ 7.4 (m, 11H), 6.4 (s, 1H), 3.7 (s, 2H), 1.3 (s, 9H).

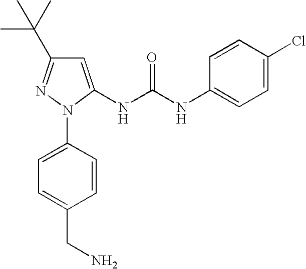

### Example 46

The title compound was synthesized in a manner analogous to Example 45 utilizing Example 44 (1.5 g, 1.5 mmol) to yield 1-{3-t-butyl-1-[4-(aminomethyl)phenyl]-1H-pyrazol-5-yl}-3-(4-chlorophenyl)urea (1.0 g). HPLC purity: 93.6%; mp: 100-102; ¹H NMR (CDCl₃): δ 8.6 (s, 1H), 7.3 (m, 8H), 6.3 (s, 1H), 3.7 (brs, 2H), 1.3 (s, 9H).

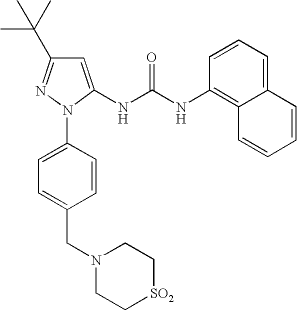

### Example 47

A 10 ml vial was changed with Example (260 mg, 63 mmol) and absolute EtOH (3 mL) under Ar. Divinylsulfone (63 uL, 74 mg, 0.63 mmol) was added drop wise over 3 min and the reaction was stirred at RT for 1.5 h. and concentrated in vacuo to yield a yellow solid, which was purified via preparative TLC, developed in 5% MeOH:CH₂Cl₂. The predominant band was cut and eluted off the silica with 1:1 EtOAc:MeOH, filtered and concentrated in vacuo to yield 1-(3-t-butyl-1-[4-(1,1-dioxothiomorpholin-4-yl)methylphenyl]-1H-pyrazol-5-yl)-3-(naphthalen-1-yl)urea (150 mg). HPLC purity: 96%; ¹H NMR (DMSO-d₆): δ 9.1 (s, 1H), 9.0 (s, 1H), 7.9 (m, 3H), 7.5 (m, 8H), 6.4 (s, 1H), 3.1 (brs, 4H), 2.9 (brs, 4H), 1.3 (s, 9H).

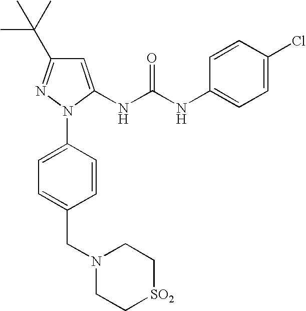

### Example 48

The title compound was synthesized in a manner analogous to Example 47 utilizing Example 46 (260 mg, 0.66 mmol) to yield 1-(3-t-butyl-1-[4-(1,1-dioxothiomorpholin-4-yl)methylphenyl]-1H-pyrazol-5-yl)-3-(4-chlorophenyl)urea (180 mg). HPLC purity: 93%; mp: 136-138; ¹H NMR (DMSO-d₆): δ 9.2 (s, 1H), 8.5 (s, 1H), 7.4 (m, 9H), 6.4 (s, 1H), 3.1 (brs, 4H), 3.0 (brs, 4H), 1.3 (s, 9H).

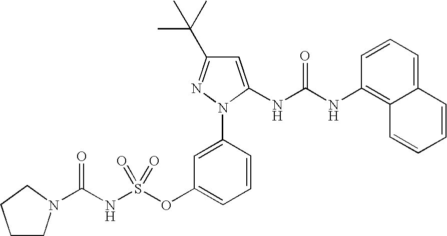

### Example 49

To a stirring solution of chlorosulfonyl isocyanate (0.35 g, 5 mmol) in CH₂Cl₂ (20 mL) at 0° C. was added pyrrolidine (0.18 g, 5 mmol) at such a rate that the reaction temperature did not rise above 5° C. After stirring for 2 h, a solution of Example 41 (1.10 g, 6.5 mmol) and triethylmine (0.46 g, 9 mmol) in CH₂Cl₂ (20 mL) was added. When the addition was complete, the mixture was allowed to warm to RT and stirred overnight. The reaction mixture was poured into 10% HCl (10 mL) saturated with NaCl, the organic layer was separated and the aqueous layer extracted with ether (20 mL). The combined organic layers were dried (Na₂SO₄) and concentrated in vacuo, purified by preparative HPLC to yield (pyrrolidine-1-carbonyl)sulfamic acid 3-[3-t-butyl-5-(3-naphthalen-1-yl-ureido)-pyrazol-1-yl]phenyl ester (40 mg). ¹H NMR (CDCl₃): δ 9.12 (brs, 1H), 8.61 (brs, 1H), 7.85-7.80 (m, 3H), 7.65 (d, J=8.0 Hz, 2H), 7.53-7.51 (m, 1H), 7.45-7.25 (m, 5H), 6.89 (s, 4H), 3.36-3.34 (brs, 1H), 3.14-3.13 (brs, 2H), 1.69 (brs, 2H), 1.62 (brs, 2H), 1.39 (s, 9H); MS (ESI) m/z: 577 (M+H⁺).

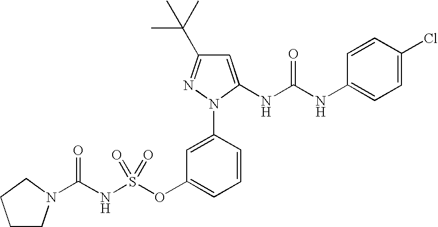

### Example 50

The title compound was synthesized in a manner analogous to Example 49 utilizing Example 42 to yield (pyrrolidine-1-carbonyl)sulfamic acid 3-[3-t-butyl-5-(4-chlorophenyl-1-yl-ureido)pyrazol-1-yl]phenyl ester. MS (ESI) m/z: 561 (M+H⁺).

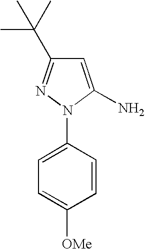

### Example W

Solid 4-methoxyphenylhydrazine hydrochloride (25.3 g) was suspended in toluene (100 mL) and treated with triethylamine (20.2 g). The mixture was stirred at RT for 30 min and treated with pivaloylacetonitrile (18 g). The reaction was heated to reflux and stirred overnight. The hot mixture was filtered, the solids washed with hexane and dried in vacuo to afford 3-t-butyl-1-(4-methoxyphenyl)-1H-pyrazol-5-amine (25 g, 70%). ¹H NMR (DMSO-d₆): δ 7.5 (d, 2H), 7.0 (d, 1H), 6.4 (s, 1H), 6.1 (s, 2H), 3.9 (s, 3H), 1.3 (s, 9H).

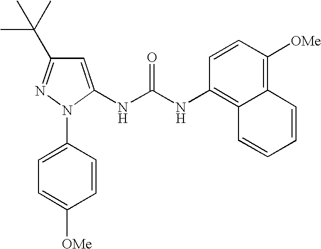

### Example 51

To a solution of 1-isocyanato-4-methoxy-naphthalene (996 mg) in anhydrous CH₂Cl₂ (20 mL) of was added Example W (1.23 g). The reaction solution was stirred for 3 h, the resulting white precipitate filtered, treated with 10% HCl and recrystallized from MeOH, and dried in vacuo to yield 1-[3-t-butyl-1-(4-methoxyphenyl)-1H-pyrazol-5-yl]-3-(1-methoxynaphthalen-4-yl-urea as white crystals (900 mg, 40%). HPLC purity: 96%; mp: 143-144; ¹H NMR (DMSO-d₆): δ 8.8 (s, 1H), 8.5 (s, 1H), 8.2 (d, 1H), 8.0 (d, 1H), 7.6 (m, 5H), 7.1 (d, 2H), 7.0 (d, 1H), 6.3 (s, 1H), 4.0 (s, 3H), 3.9 (s, 3H); 1.3 (s, 9H).

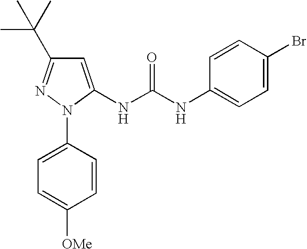

### Example 52

The title compound was synthesized in a manner analogous to Example 51 utilizing Example W and p-bromophenylisocyanate (990 mg) to yield 1-(3-t-butyl-1-(4-methoxyphenyl)-1H-pyrazol-5-yl)-3-(4-bromophenyl)urea as off-white crystals (1.5 g, 68%). HPLC purity: 98%; mp: 200-201; ¹H NMR (DMSO-d₆): δ 9.3 (s, 1H), 8.3 (s, 1H), 7.4 (m, 6H), 7.0 (d, 2H), 6.3 (s, 1H), 3.8 (s, 3H), 1.3 (s, 9H).

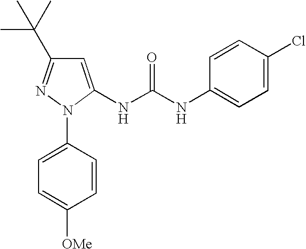

### Example 53

The title compound was synthesized in a manner analogous to Example 51 utilizing Example W and p-chlorophenylisocyanate (768 mg) into yield 1-{3-t-butyl-1-(4-methoxyphenyl)-1H-pyrazol-5-yl}-3-(4-chlorophenyl)urea as white crystals (1.3 g, 65%). HPLC purity: 98%; mp: 209-210; ¹H NMR (DMSO-d₆): δ 9.1 (s, 1H), 8.3 (s, 1H), 7.4 (m, 4H), 7.3 (d, 2H), 7.1 (d, 2H), 6.3 (s, 1H), 3.8 (s, 3H), 1.3 (s, 9H).

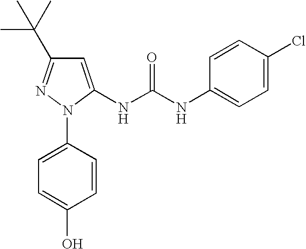

### Example 54

The title compound was synthesized in a manner analogous to Example 41 utilizing Example 53 (500 mg) to yield 1-{3-t-butyl-1-(4-hydroxyphenyl)-1H-pyrazol-5-yl}-3-(4-chlorophenyl)urea as white crystals (300 mg, 62%). HPLC purity: 94%; mp: 144-145; ¹H NMR (DMSO-d₆): δ 9.7 (s, 1H), 9.1 (s, 1H), 8.3 (s, 1H), 7.4 (d, 2H), 7.3 (m, 4H); 6.9 (d, 2H), 6.3 (s, 1H), 1.3 (s, 9H)

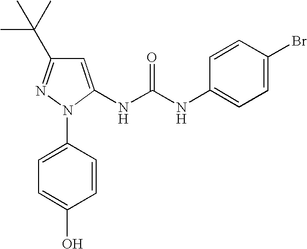

### Example 55

The title compound was synthesized in a manner analogous to Example 41 utilizing Example 52 (550 mg) to yield 1-{3-t-butyl-1-(4-hydroxyphenyl)-1H-pyrazol-5-yl}-3-(4-bromophenyl)urea as a white crystalline solid (400 mg, 70%). HPLC purity: 93%; mp: 198 200; ¹H NMR (DMSO-d₆): δ 9.7 (s, 1H), 9.2 (s, 1H), 9.3 (s, 1H), 7.4 (d, 4H), 7.2 (m, 2H), 6.9 (d, 2H), 6.3 (s, 1H), 1.3 (s, 9H).

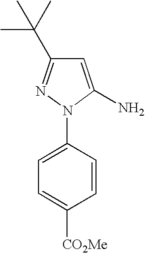

### Example X

Methyl 4-(3-t-butyl-5-amino-1H-pyrazol-1-yl)benzoate (3.67 mmol) was prepared from methyl 4-hydrazinobenzoate and pivaloylacetonitrile by the procedure of Regan, et al., J. Med. Chem., 45, 2994 (2002).

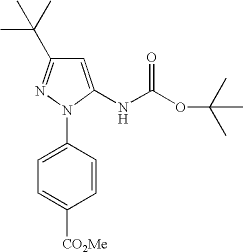

### Example 56

A 500 mL round bottom flask was equipped with a magnetic stir bar and an ice bath. The flask was charged with Example X (1 g) and this was dissolved in CH₂Cl₂ (100 mL). Saturated sodium bicarbonate (100 mL) was added and the mixture rapidly stirred, cooled in an ice bath and treated with diphosgene (1.45 g) and the heterogeneous mixture stirred for 1 h. The layers were separated and the CH₂Cl₂ layer treated with t-butanol (1.07 g) and the solution stirred overnight at RT. The solution was washed with H₂O (2×150 mL), dried (Na₂SO₄), filtered, concentrated in vacuo, and purified by flash chromatography using 1:2 ethyl acetate:hexane as the eluent to yield t-butyl 1-(4-(methoxycarbonyl)phenyl)-3-t-butyl-1H-pyrazol-5-ylcarbamate (100 mg) as an off-white solid. ¹H NMR (DMSO-d₆): δ 9.2 (s, 1H), 8.1 (d, 2H), 7.7 (d, 2H), 6.3 (s, 1H), 3.3 (s, 3H), 1.3 (s, 18H).

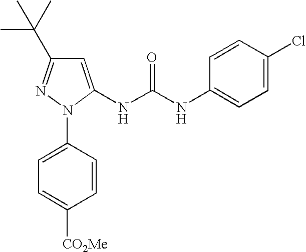

### Example 57

The title compound was synthesized in a manner analogous to Example 41 utilizing Example X (1.37 g) and p-chlorophenylisocyanate (768 mg) to yield methyl 4-{3-t-butyl-5-[3-(4-chlorophenyl)ureido]-1H-pyrazol-1-yl}benzoate as white crystals (1.4 g 66%). HPLC purity: 98%; mp: 160-161; ¹H NMR (DMSO-d₆): δ 9.2 (s, 1H), 8.6 (s, 1H), 8.1 (d, 2H), 7.8 (d, 2H), 7.5 (d, 2H), 7.3 (d, 2H), 6.4 (s, 1H), 3.9 (s, 3H), 1.3 (s, 9H).

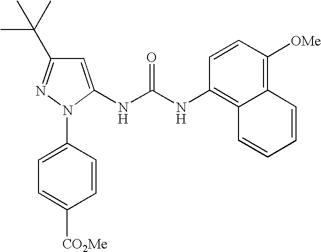

### Example 58

The title compound was synthesized in a manner analogous to Example 41 utilizing Example X (1.27 g) and 1-isocyanato-4-methoxy-naphthalene (996 mg) to yield methyl 4-{3-t-butyl-5-[3-(1-methoxynaphthalen-4-yl)ureido]-1H-pyrazol-1-yl}benzoate as white crystals (845 mg, 36%). HPLC purity: 98%; mp: 278 280; ¹H NMR (DMSO-d₆): δ 8.76 (s, 1H), 8.73 (s, 1H), 8.1 (m, 3H), 7.9 (d, 1H), 7.7 (d, 2H), 7.6 (m, 3H), 7.0 (d, 1H), 7.0 (d, 1H), 6.3 (s, 1H), 4.0 (s, 3H), 3.9 (s, 3H), 1.3 (s, 9H).

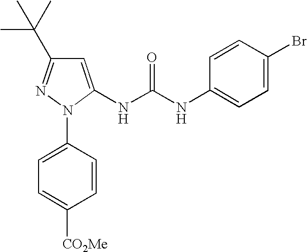

### Example 59

The title compound was synthesized in a manner analogous to Example 41 utilizing Example X (1.37 g) and p-bromophenylisocyanate (990 mg) to yield methyl 4-{3-t-butyl-5-[3-(4-bromophenyl)ureido]-1H-pyrazol-1-yl}benzoate as white crystals (1.4 g, 59%). HPLC purity: 94%; mp: 270 272; ¹H NMR (DMSO-d₆): δ 9.2 (s, 1H), 8.6 (s, 1H), 8.1 (d, 2H), 7.7 (d, 2H), 7.4 (d, 4H), 6.4 (s, 1H), 3.9 (s, 3H), 1.3 (s, 9H).

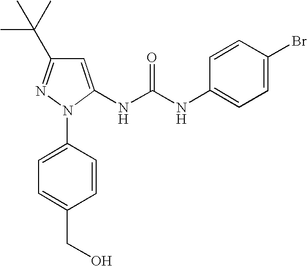

### Example 60

To a solution of Example 59 (700 mg) in 30 mL of toluene at −78° C., was added dropwise a solution of diisobutylaluminum hydride in toluene (1M in toluene, 7.5 mL) over 10 min. The reaction mixture was stirred for 30 min at −78° C., and then 30 min at 0° C. The reaction mixture was concentrated in vacuo to dryness and treated with H₂O. The solid was filtered and treated with acetonitrile. The solution was evaporated to dryness and the residue was dissolved in ethyl acetate, and precipitated by hexanes to afford yellow solid which was dried under vacuum to give 1-[3-t-butyl-1-(4-hydroxymethyl)phenyl)-1H-pyrazol-5-yl]urea (400 mg, 61%). HPLC purity: 95%; ¹H NMR (DMSO-d₆): δ 9.2 (s, 1H), 8.4 (s, 1H), 7.5 (m, 8H), 6.4 (s, 1H), 5.3 (t, 1H), 4.6 (d, 2H), 1.3 (s, 9H).

### Example 1A

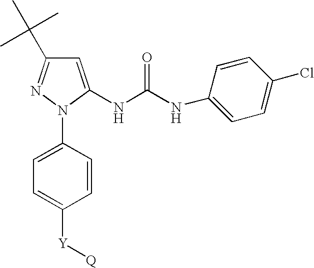

### Example 2A

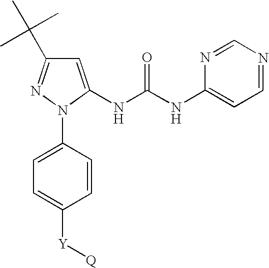

### Example 3A

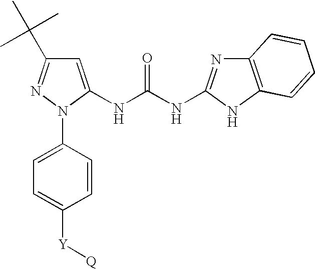

### Example 4A

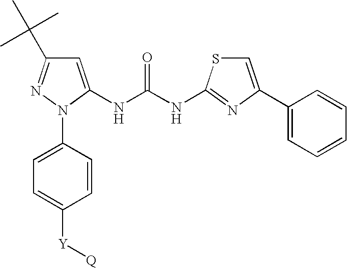

### Example 5A

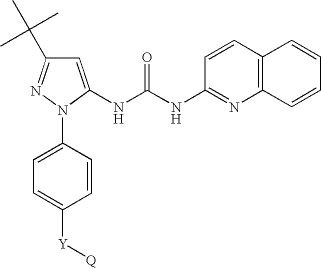

### Example 6A

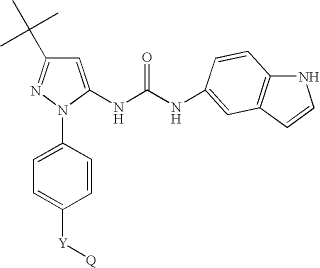

Wherein Y is O, S, NR6, —NR6SO2-, NR6CO—, alkylene, O—(CH2)ₙ—, NR6-(CH2)n-, wherein one of the methylene units may be substituted with an oxo group, or Y is a direct bond; Q is taken from the groups identified in Chart I:

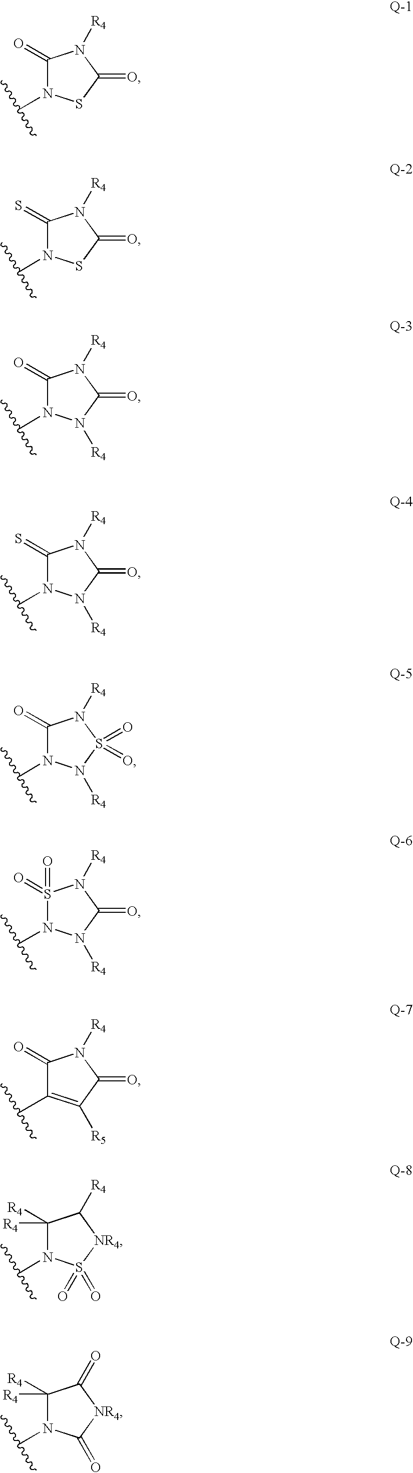

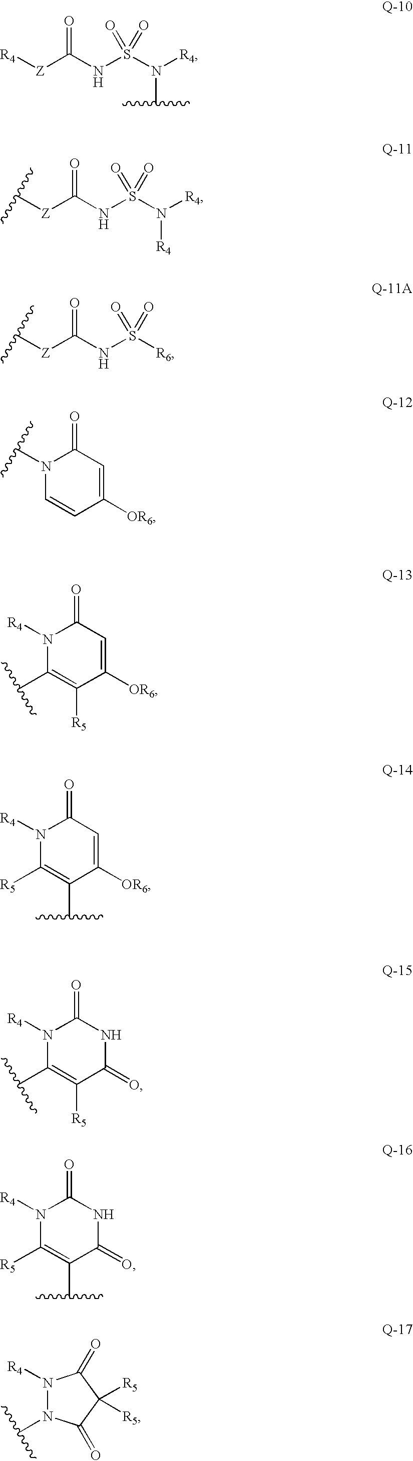

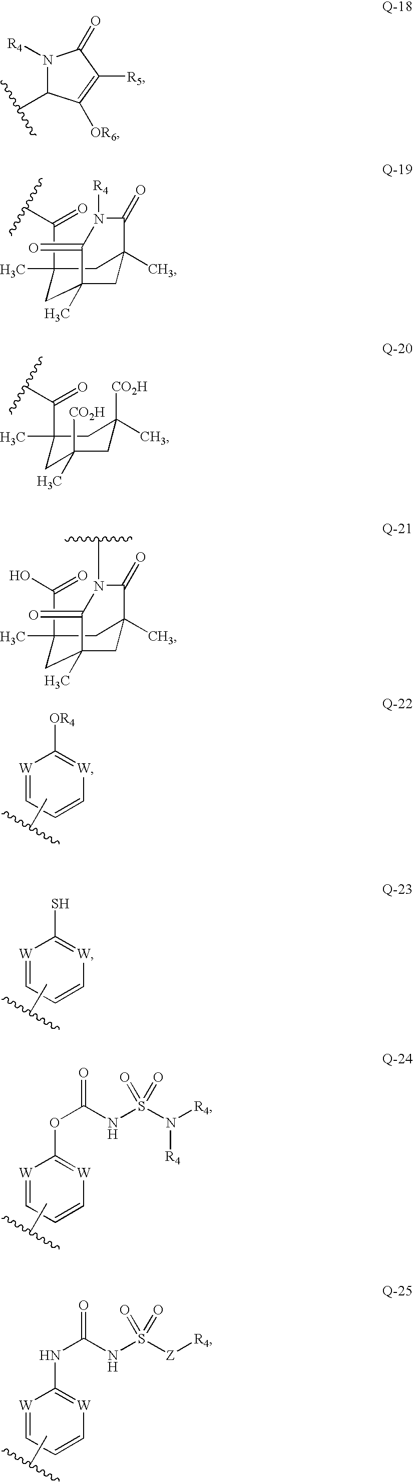

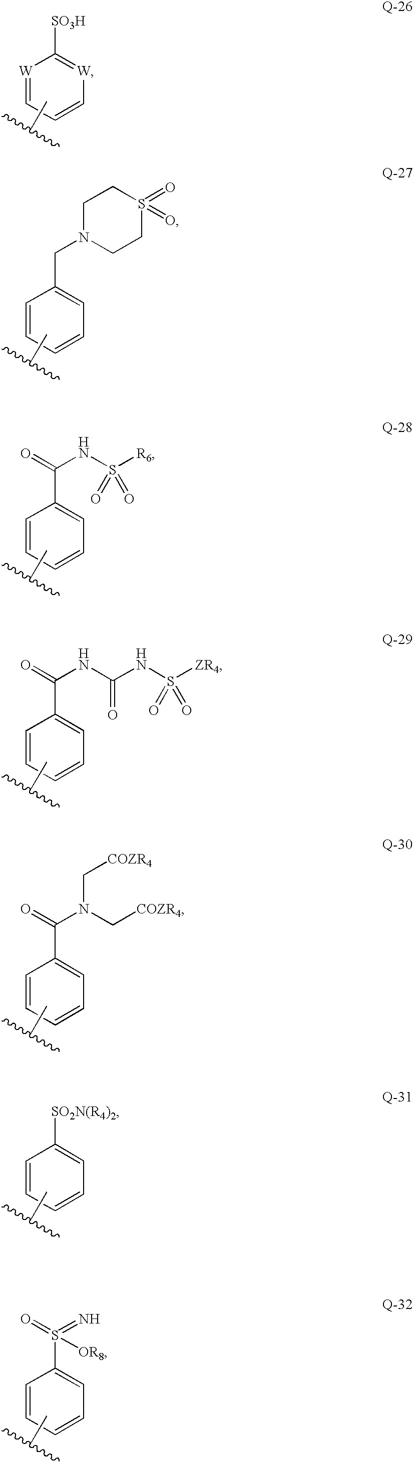

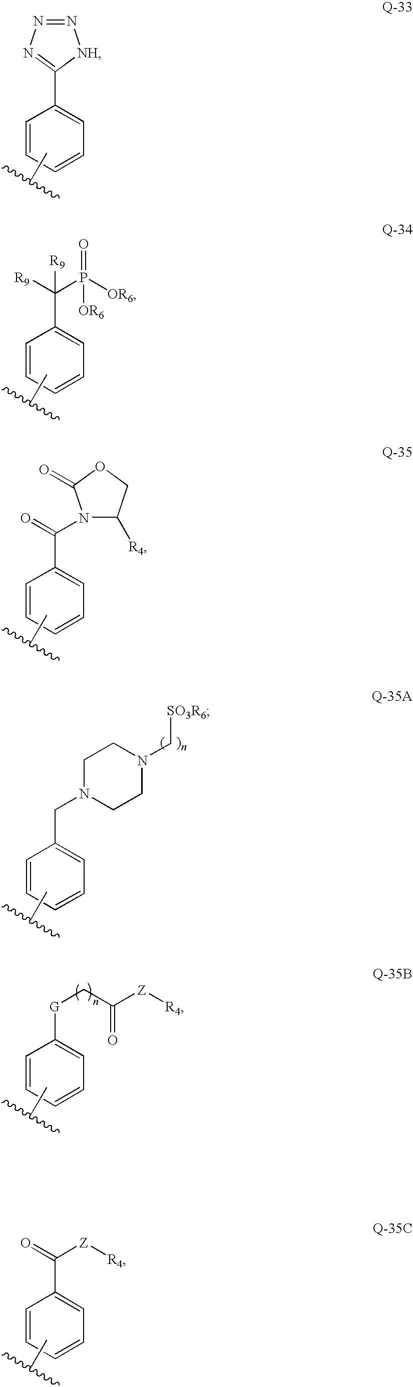

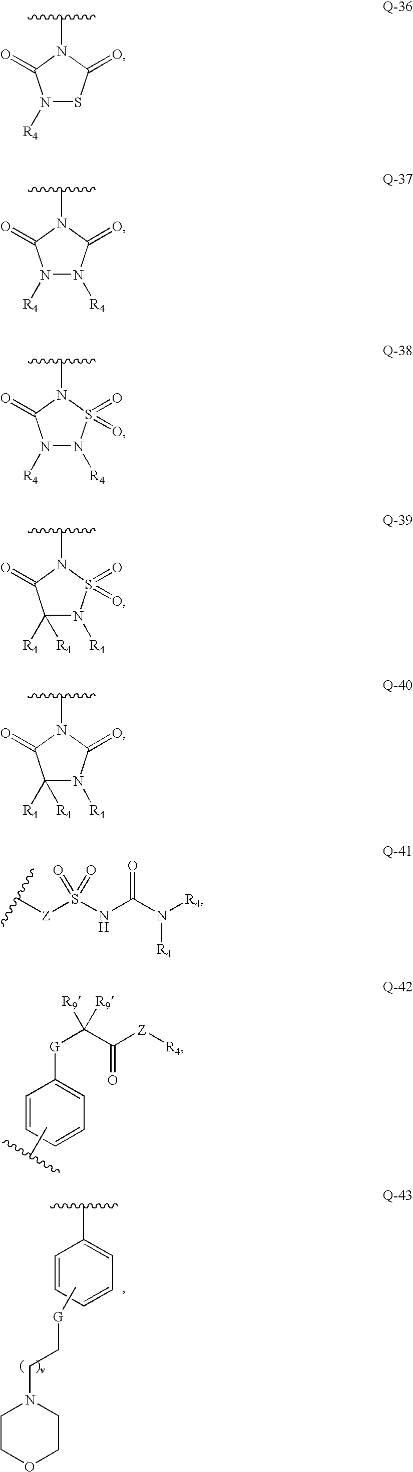

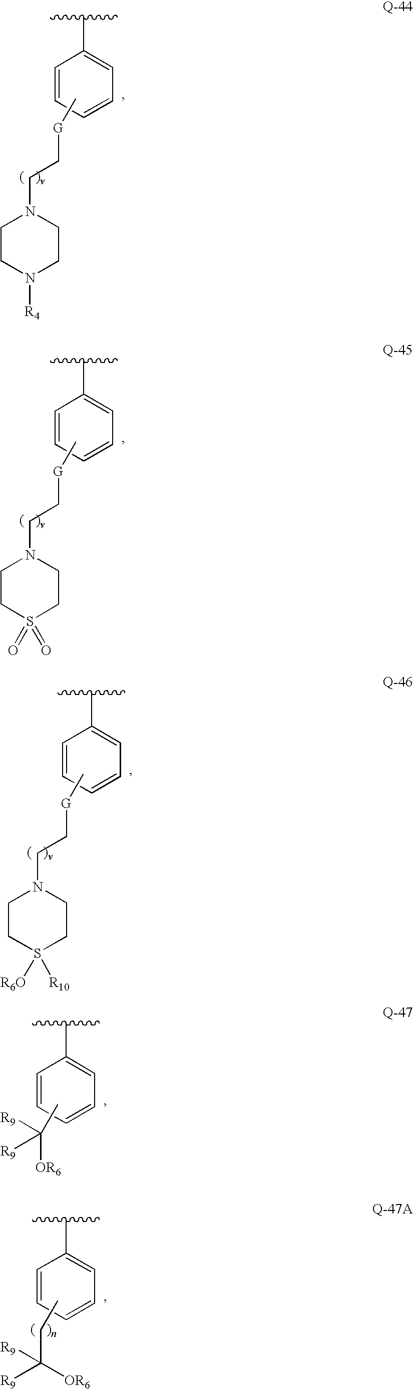

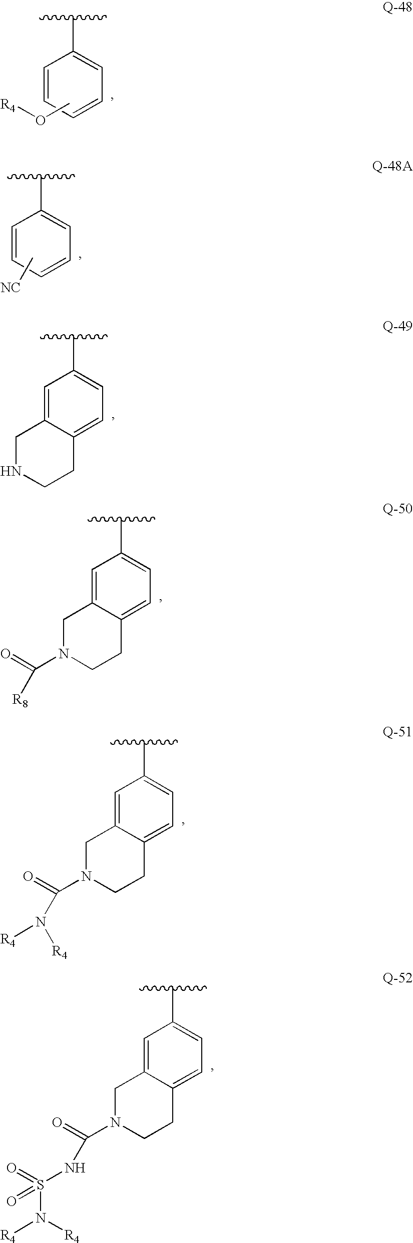

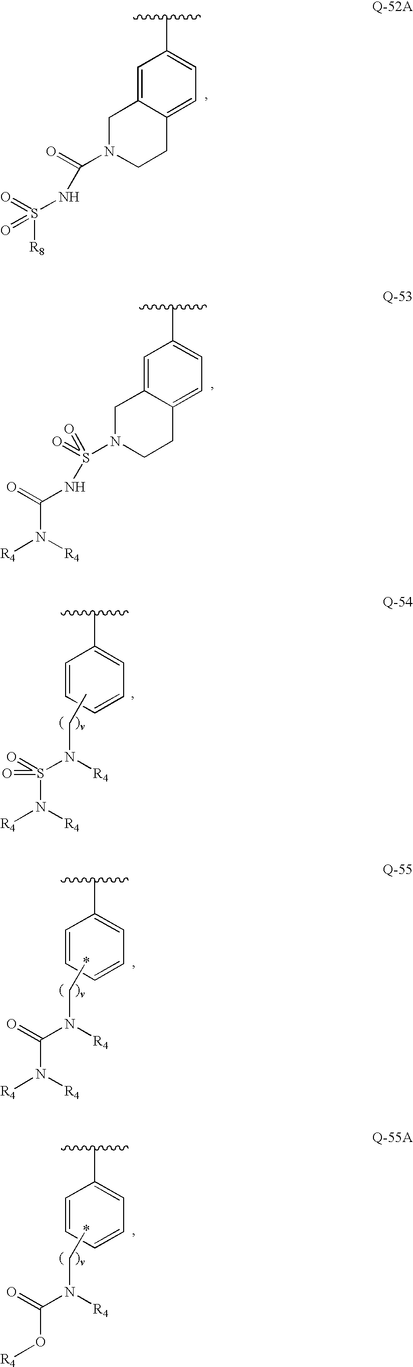

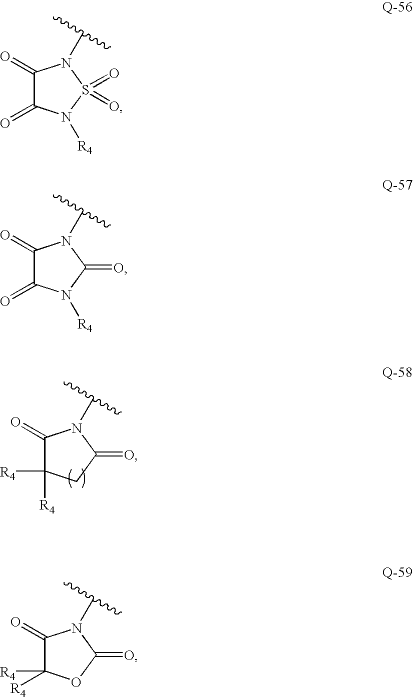

### Example 7A

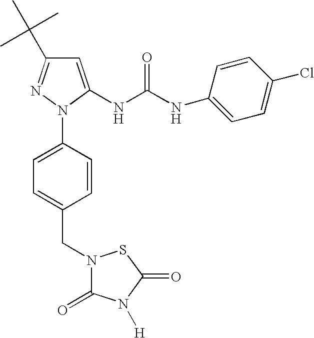

### Example 8A

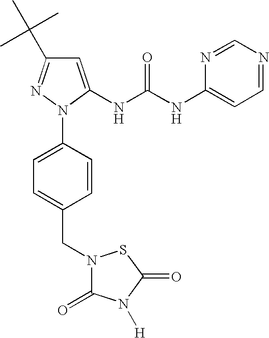

### Example 9A

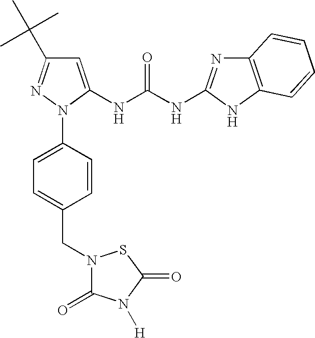

### Example 10A

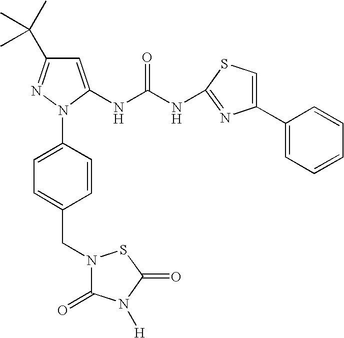

### Example 11A

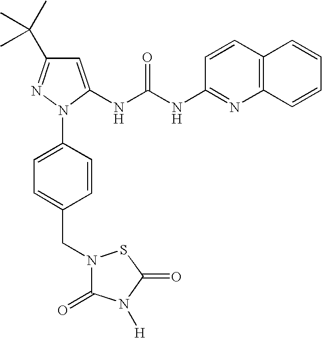

### Example 12A

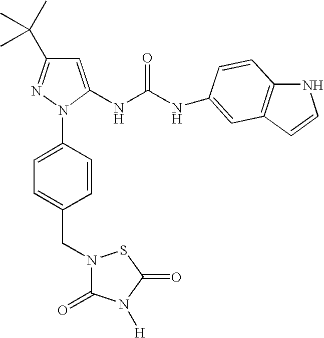

### Example 13A

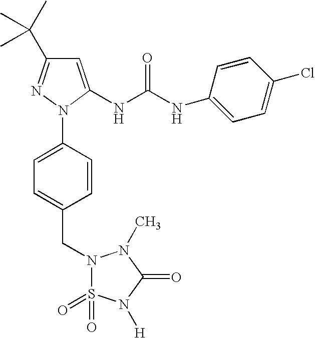

### Example 14A

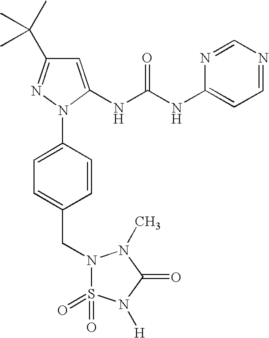

### Example 15A

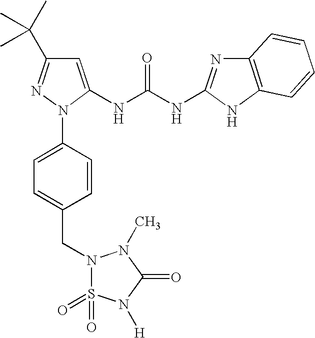

### Example 16A

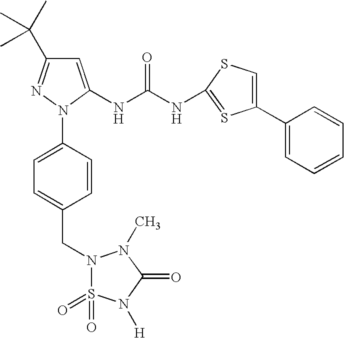

### Example 17A

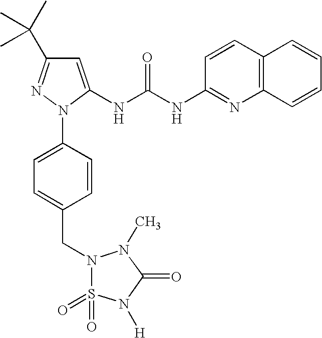

### Example 18A

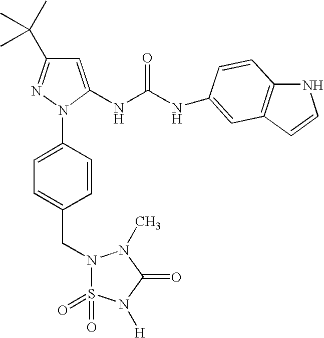

### Example 19A

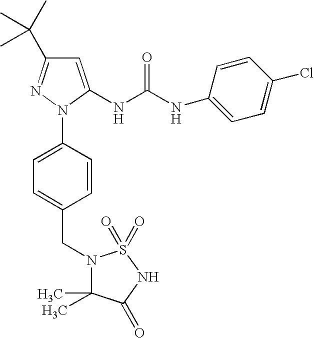

### Example 20A

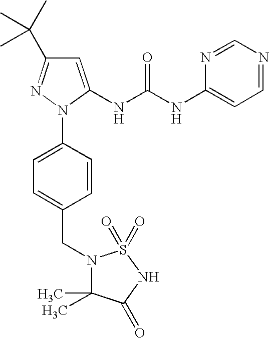

### Example 21A

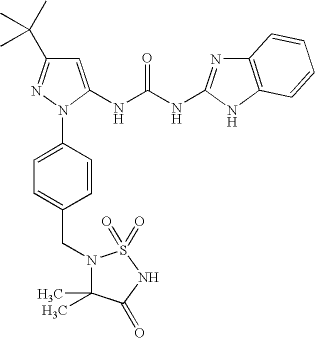

### Example 22A

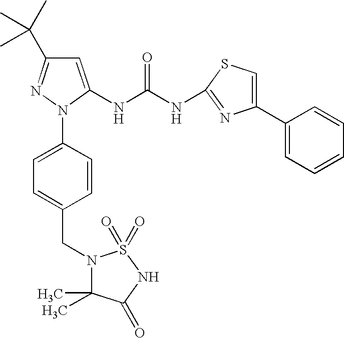

### Example 23A

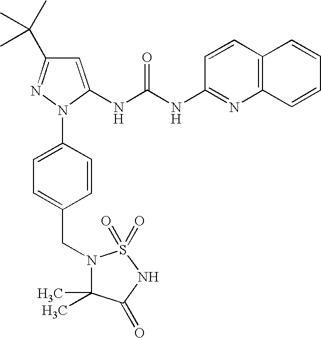

### Example 24A

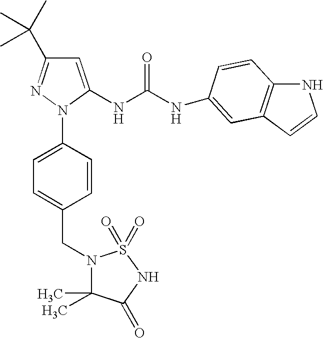

### Example 25A

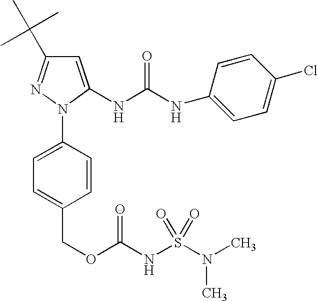

### Example 26A

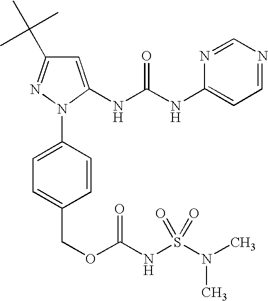

### Example 27A

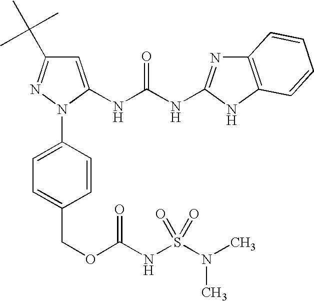

### Example 28A

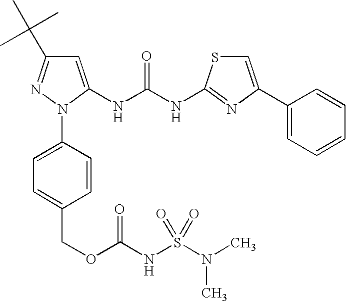

### Example 29A

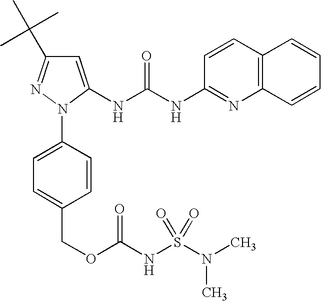

### Example 30A

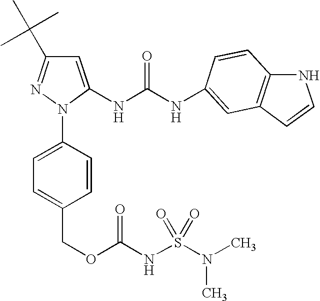

### Example 31A

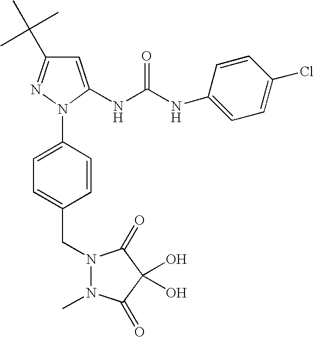

### Example 32A

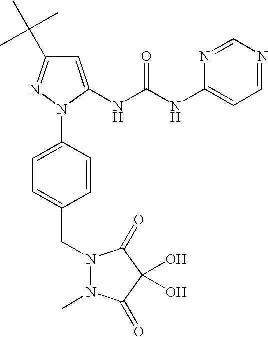

### Example 33A

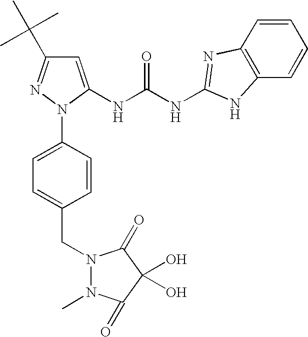

### Example 34A

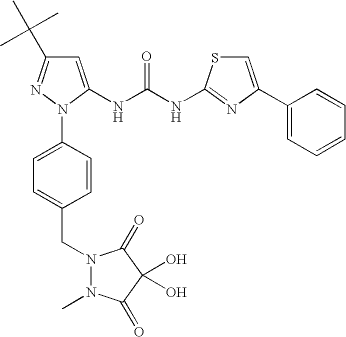

### Example 35A

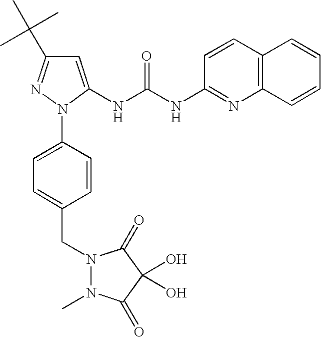

### Example 36A

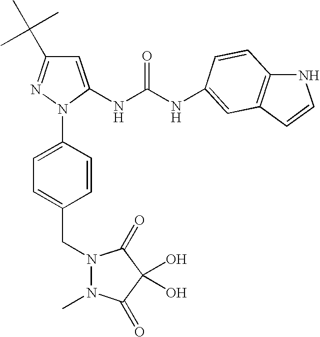

### Example 37A

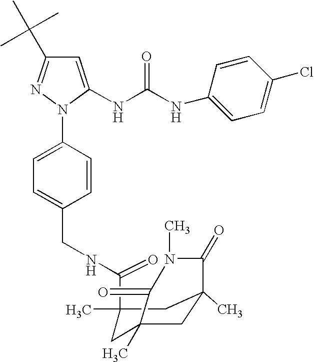

### Example 38A

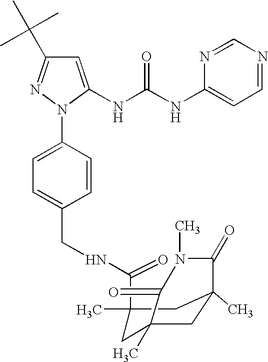

### Example 39A

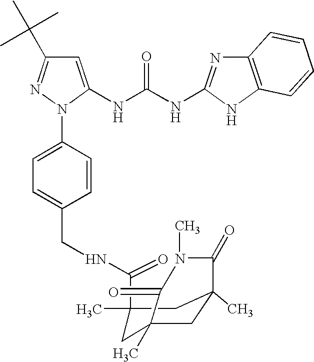

### Example 40A

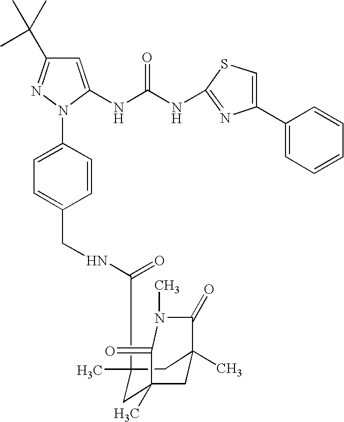

### Example 41A

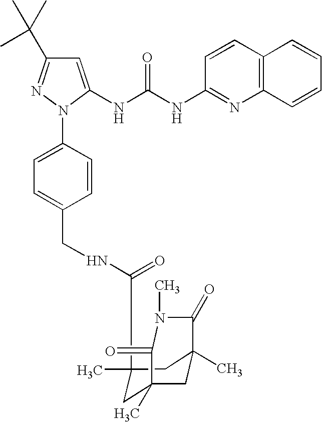

### Example 42A

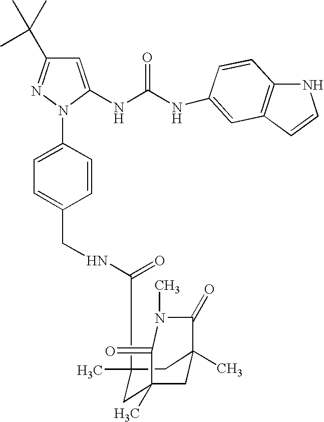

### Example 43A

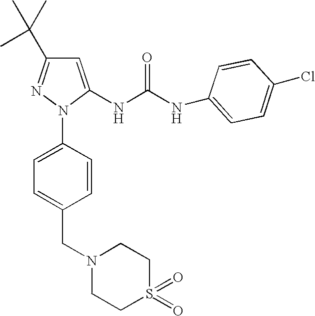

### Example 44A

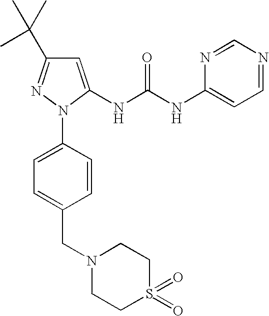

### Example 45A

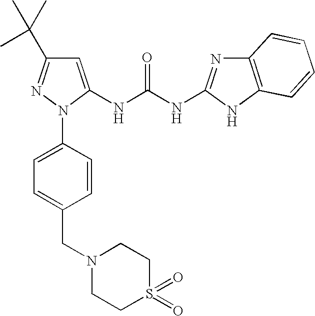

### Example 46A

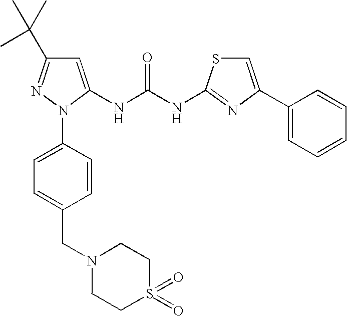

### Example 47A

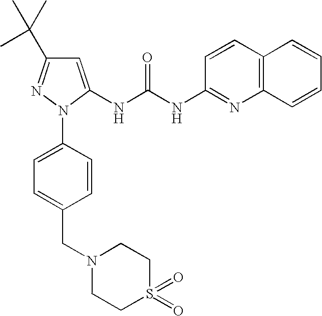

### Example 48A

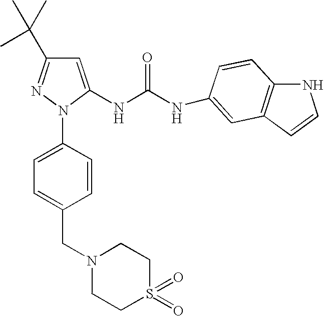

### Example 49A

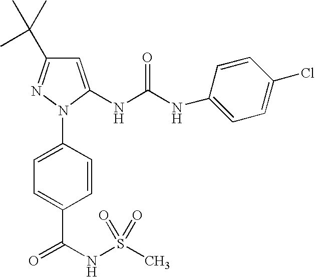

### Example 50A

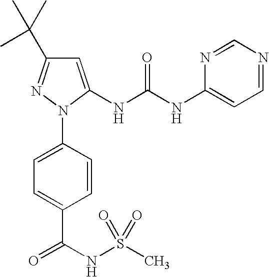

### Example 51A

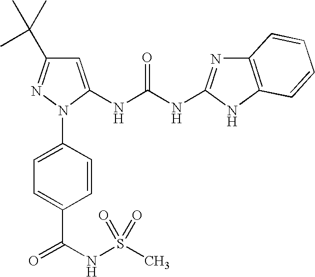

### Example 52A

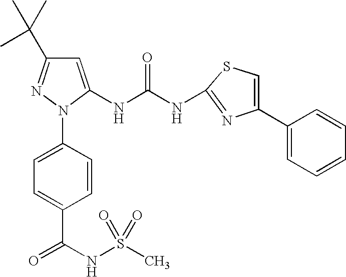

### Example 53A

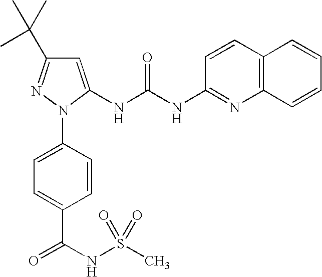

### Example 54A

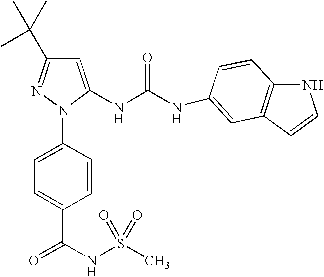

### Example 55A

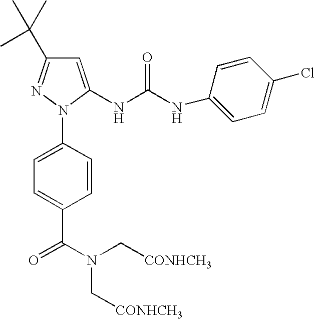

### Example 56A

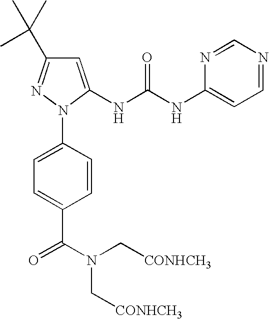

### Example 57A

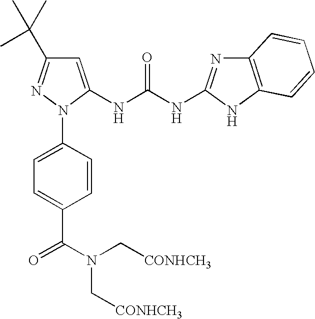

### Example 58A

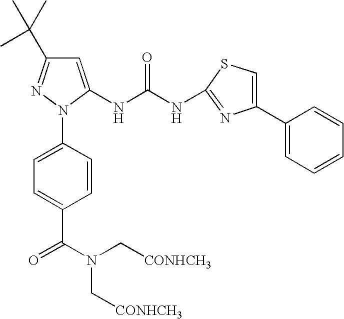

### Example 59A

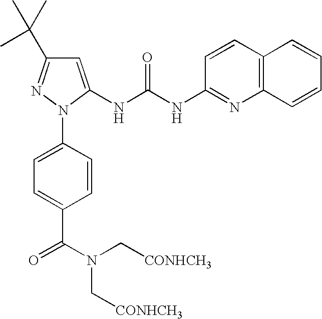

### Example 60A

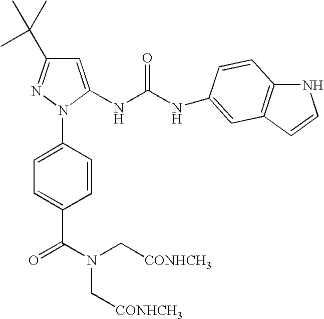

### Example 61

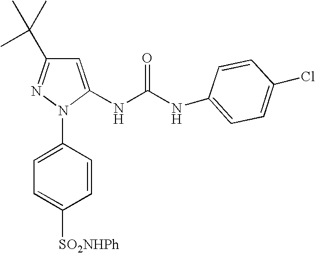

### Example 62

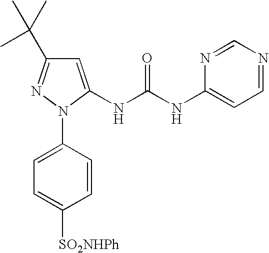

### Example 63

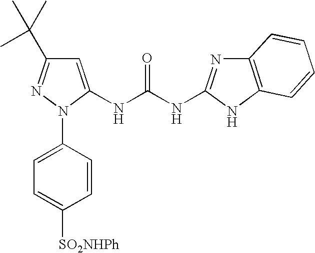

### Example 64

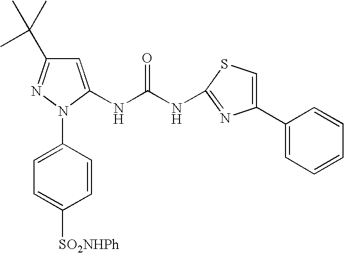

### Example 65

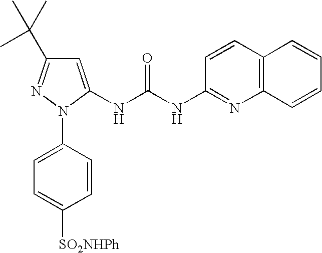

### Example 66

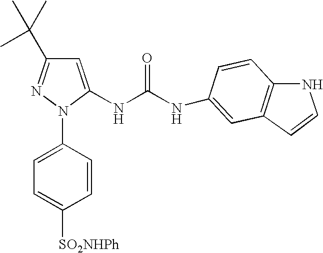

### Example 67

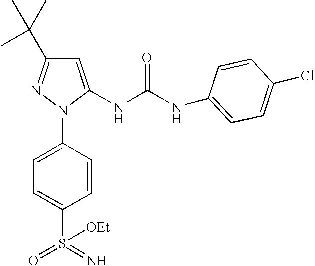

### Example 68

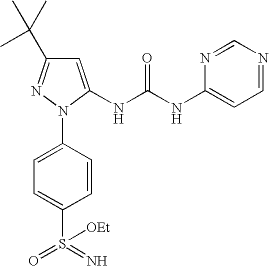

### Example 69

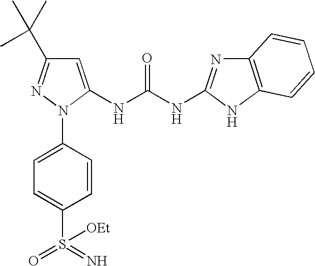

### Example 70

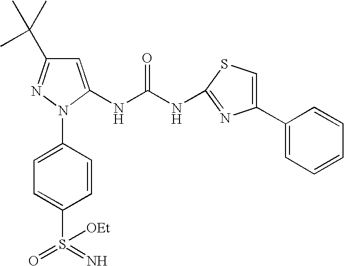

### Example 71

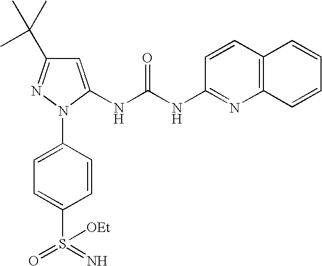

### Example 72

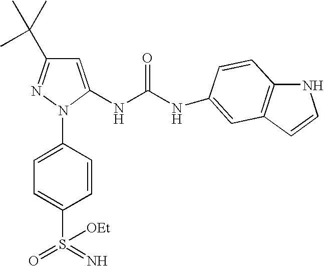

### Example 73

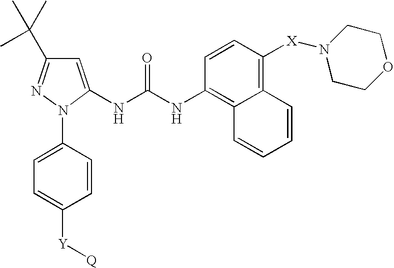

### Example 74

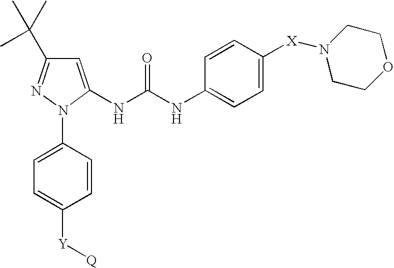

### Example 75

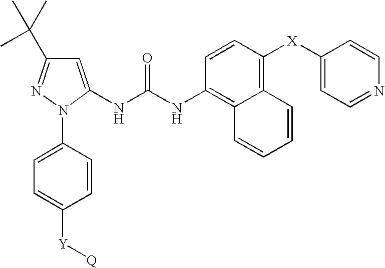

### Example 76

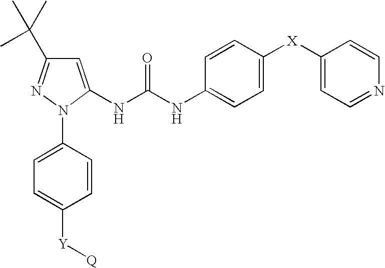

### Example 77

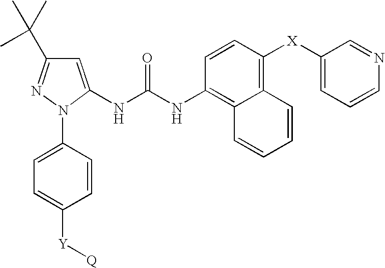

### Example 78

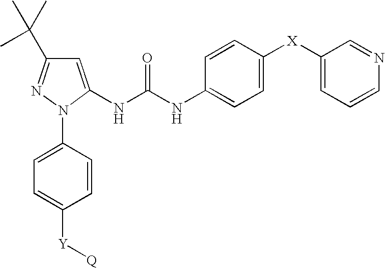

### Example 79

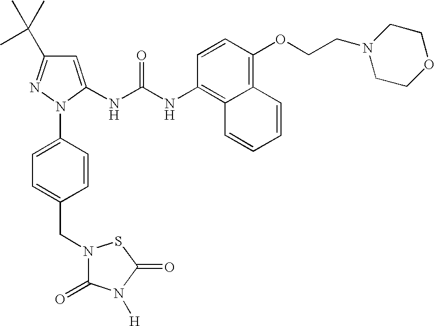

### Example 80

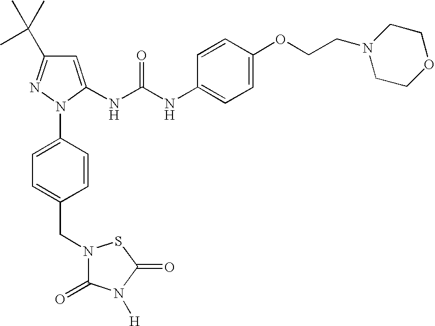

### Example 81

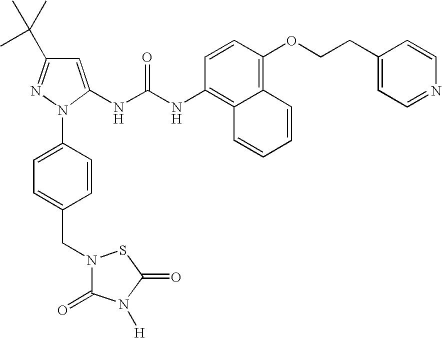

### Example 82

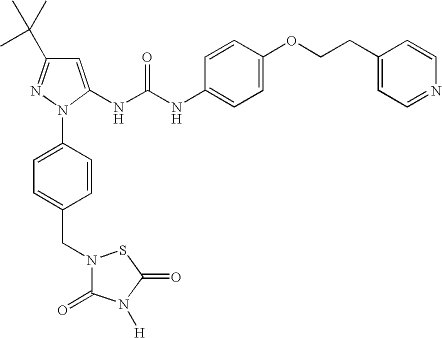

### Example 83

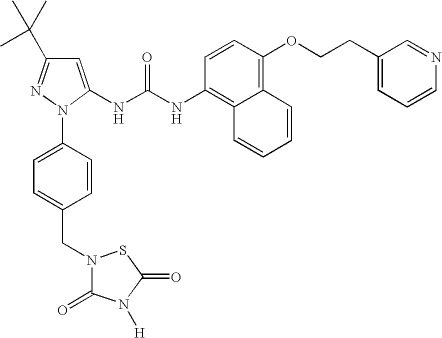

### Example 84

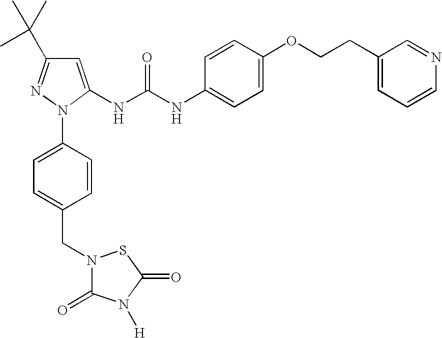

### Example 85

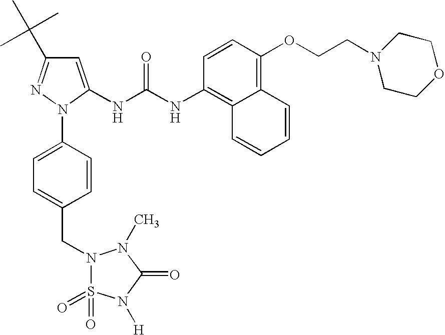

### Example 86

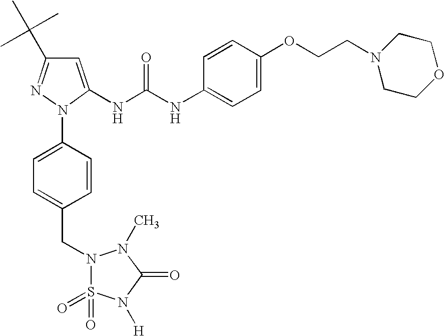

### Example 87

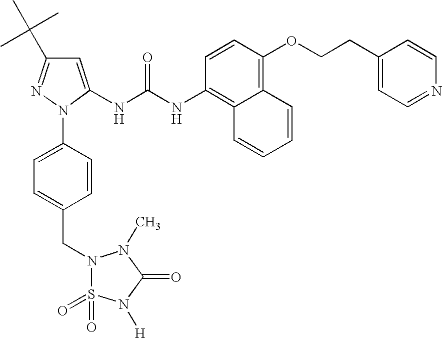

### Example 88

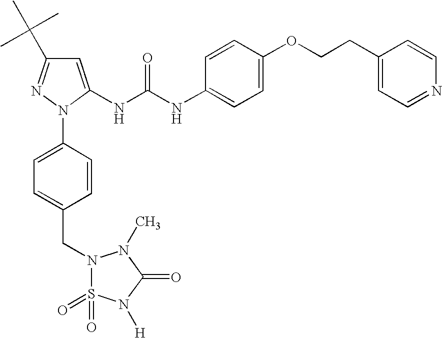

### Example 89

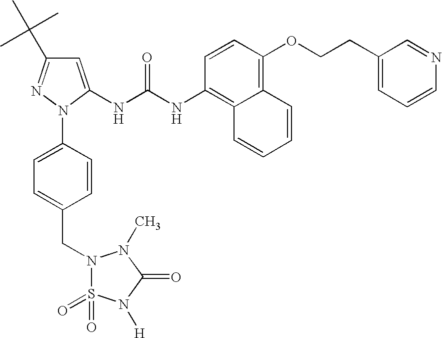

### Example 90

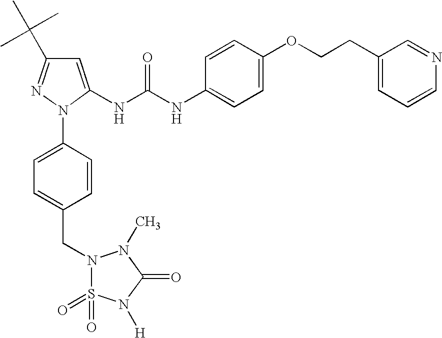

### Example 91

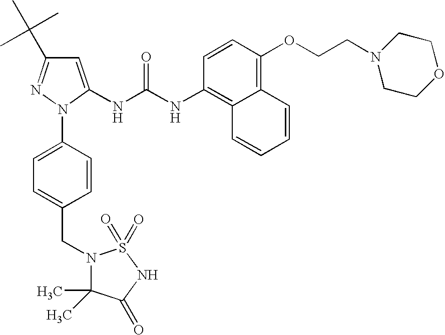

### Example 92

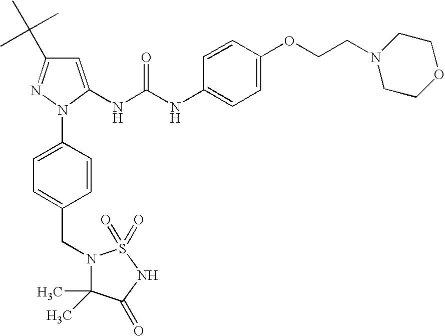

### Example 93

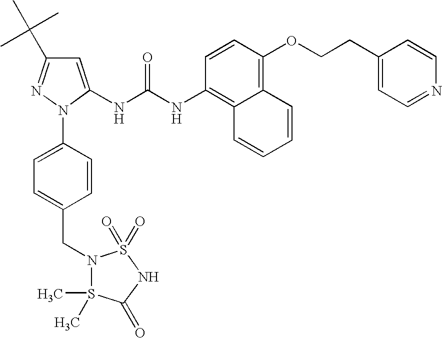

### Example 94

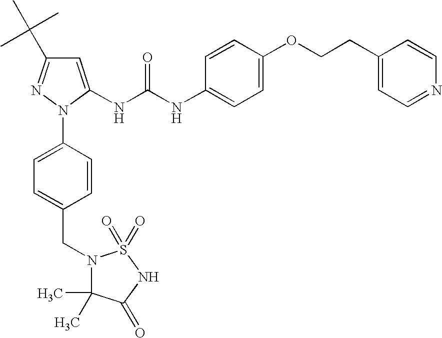

### Example 95

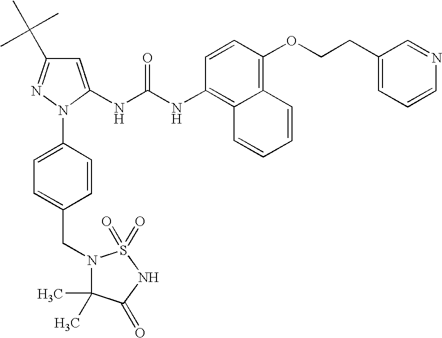

### Example 96

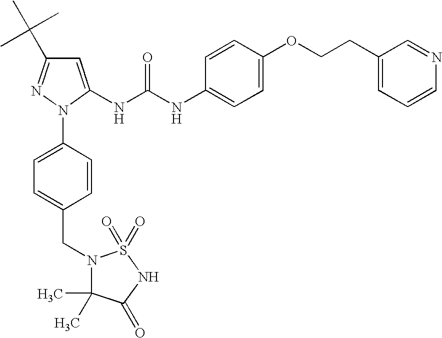

### Example 97

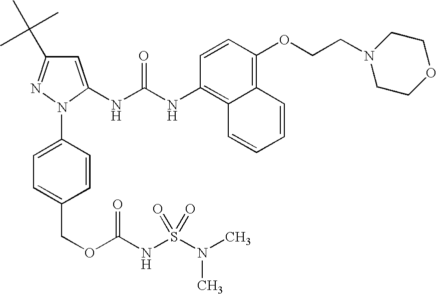

### Example 98

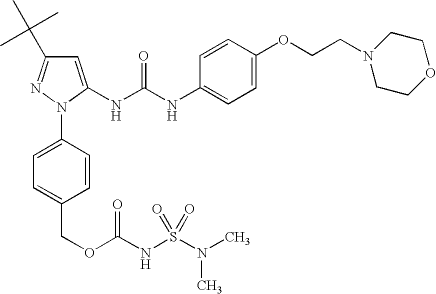

### Example 99

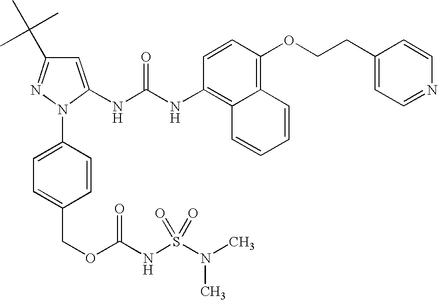

### Example 100

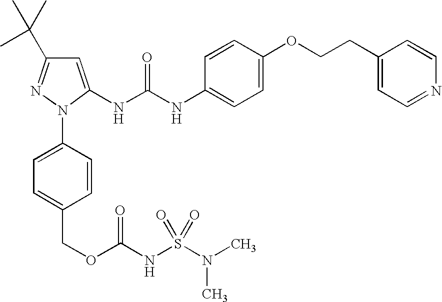

### Example 101

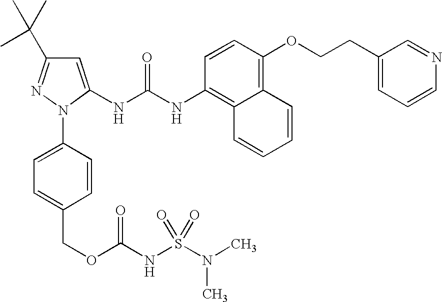

### Example 102

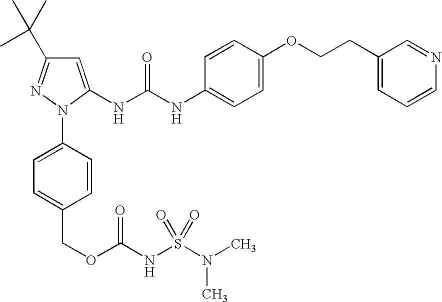

### Example 103

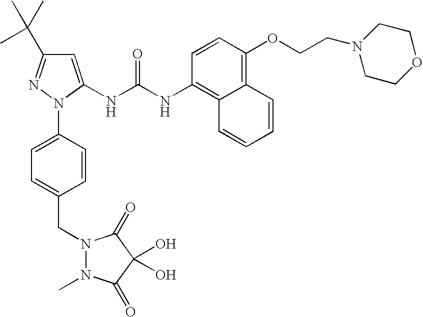

### Example 104

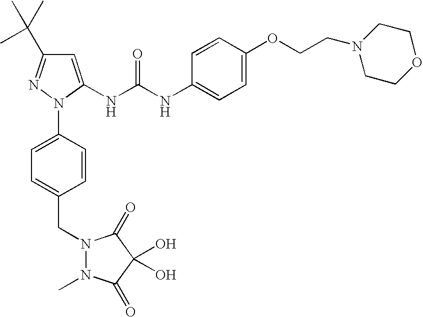

### Example 105

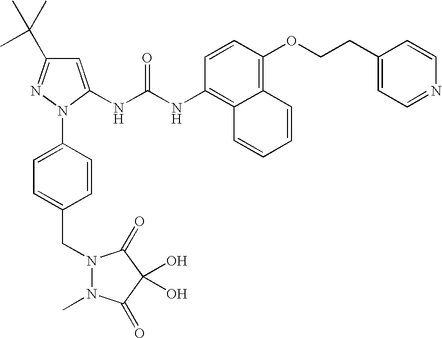

### Example 106

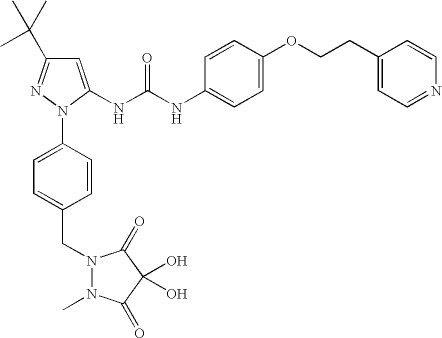

### Example 107

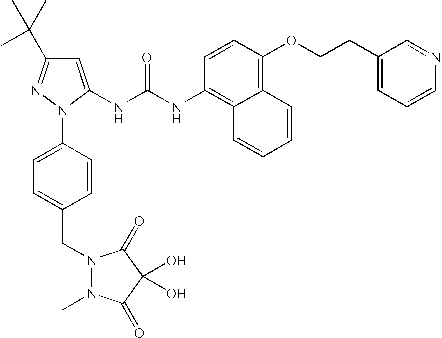

### Example 108

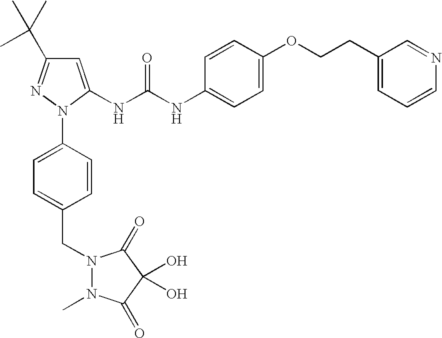

### Example 109

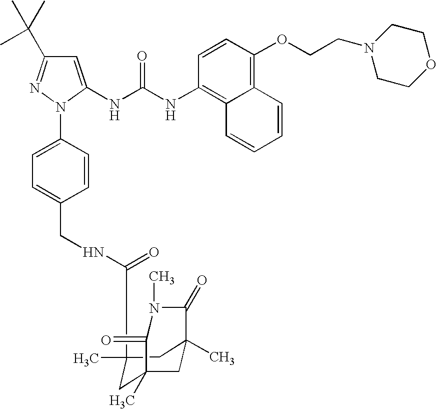

### Example 110

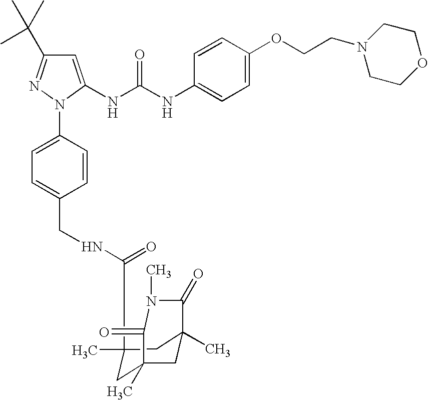

### Example 111

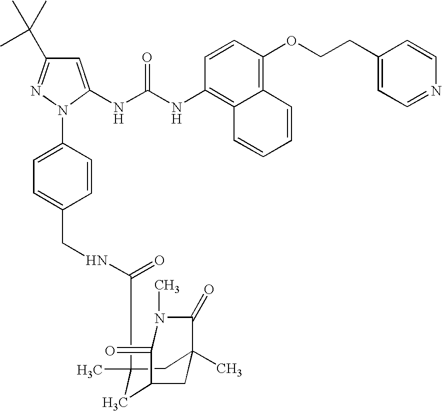

### Example 112

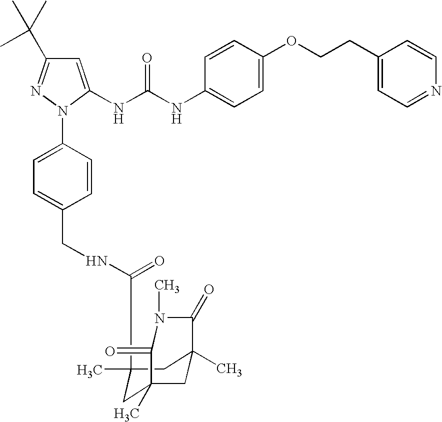

### Example 113

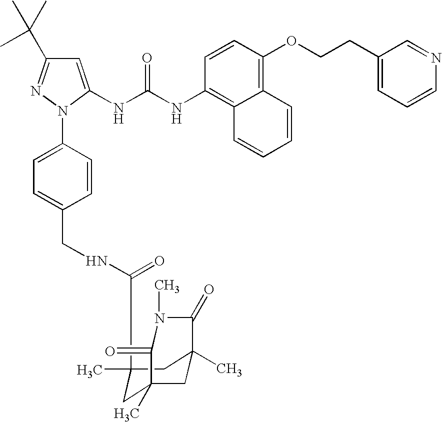

### Example 114

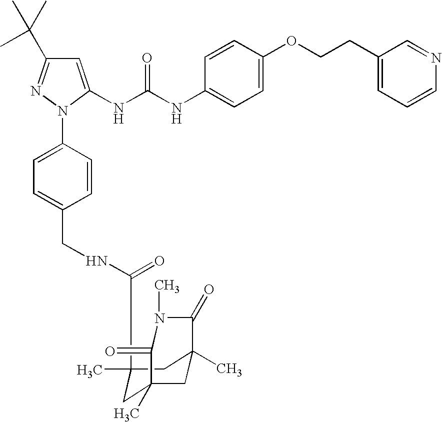

### Example 115

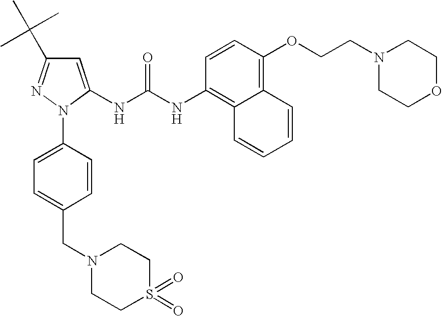

### Example 116

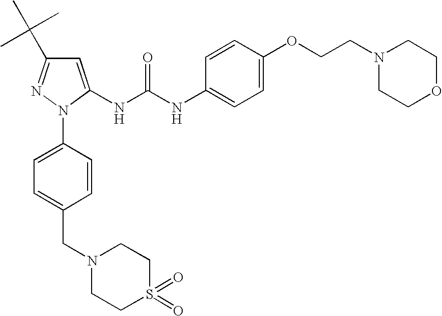

### Example 117

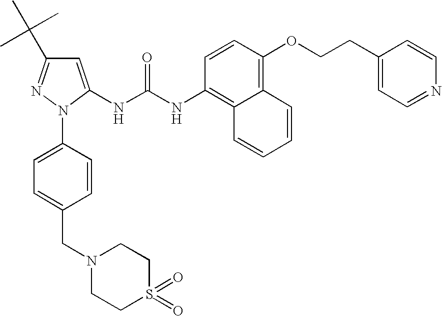

### Example 118

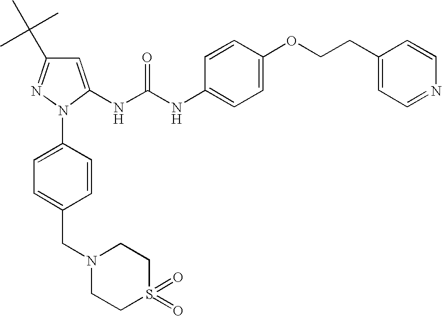

### Example 119

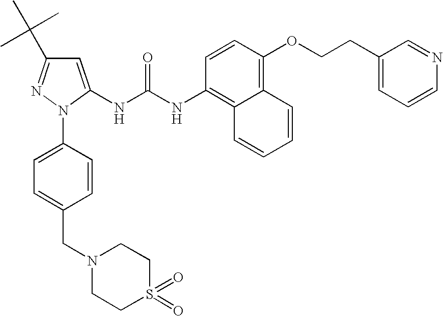

### Example 120

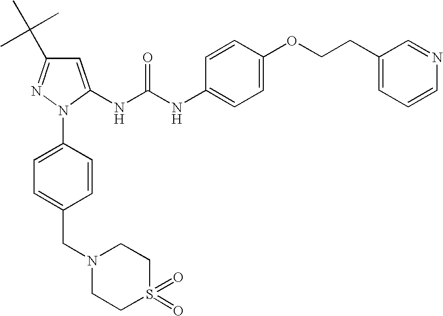

### Example 121

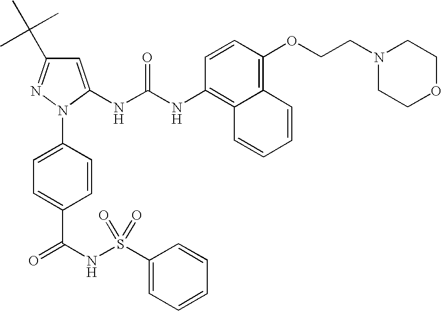

### Example 122

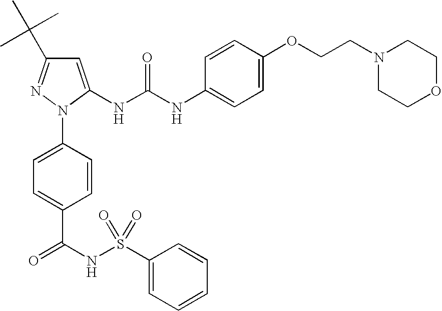

### Example 123

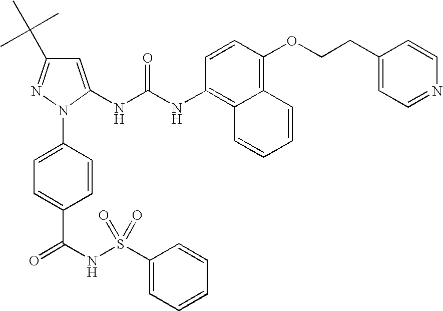

### Example 124

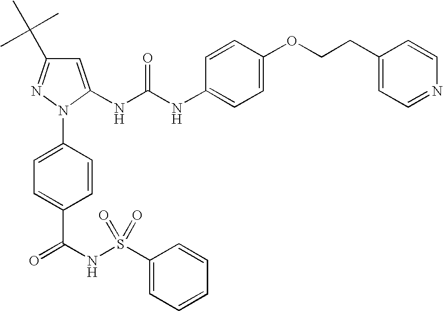

### Example 125

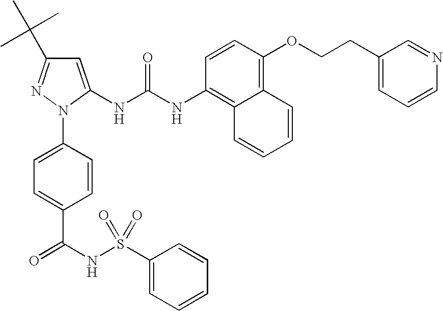

### Example 126

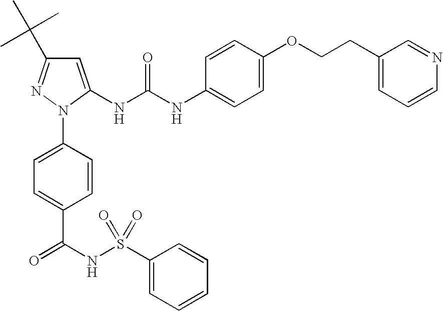

### Example 127

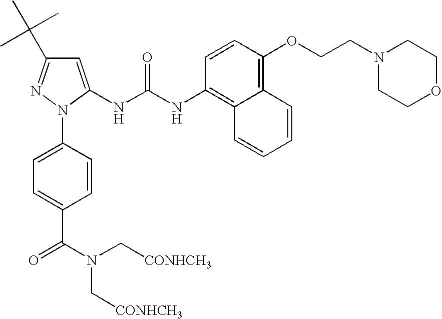

### Example 128

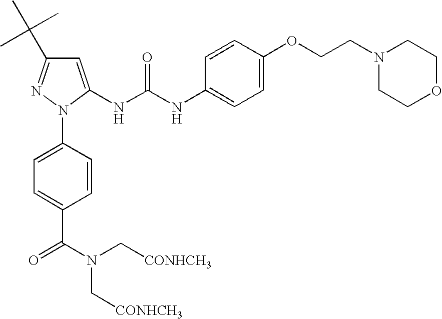

### Example 129

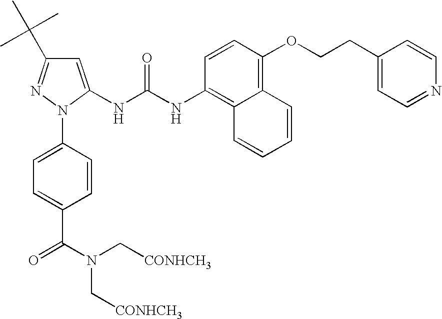

### Example 130

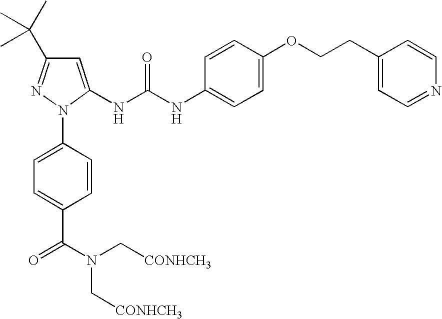

### Example 131

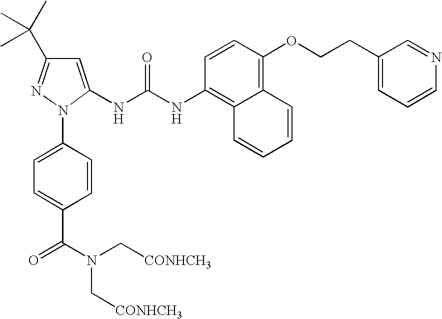

### Example 132

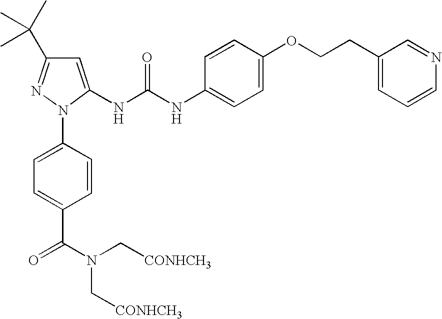

### Example 133

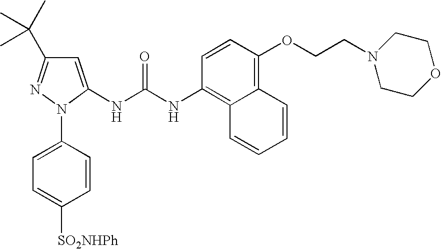

### Example 134

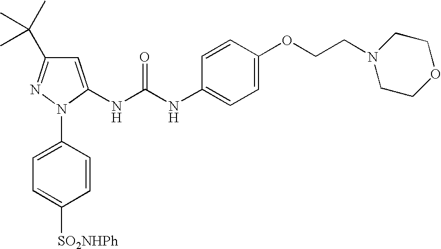

### Example 135

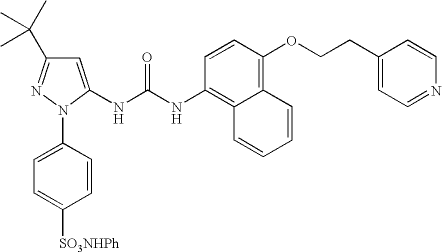

### Example 136

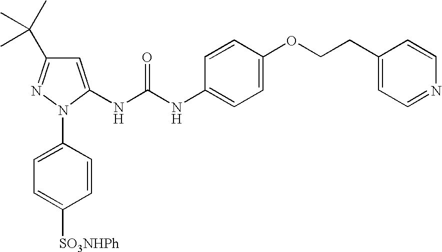

### Example 137

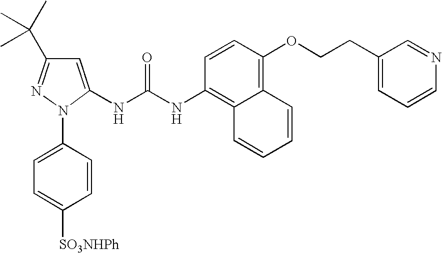

### Example 138

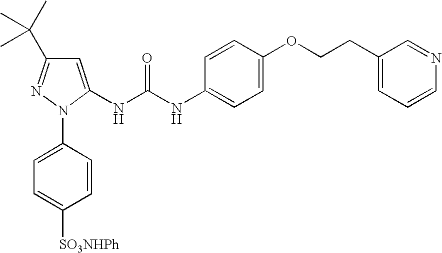

### Example 139

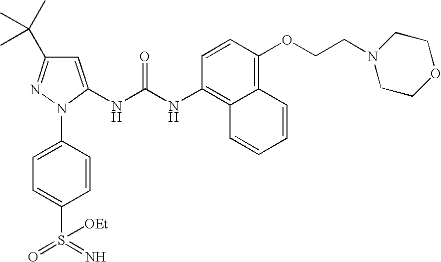

### Example 140

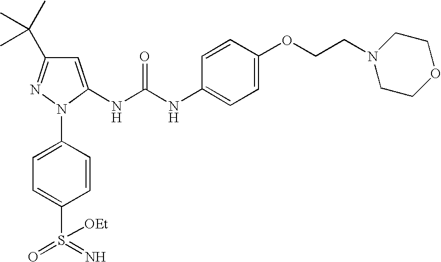

### Example 141

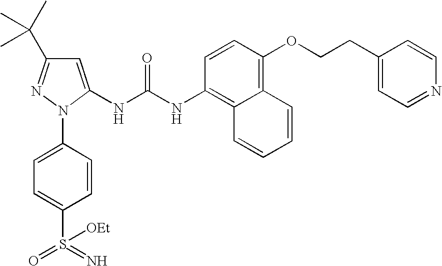

### Example 142

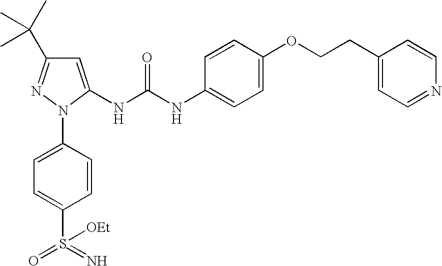

### Example 143

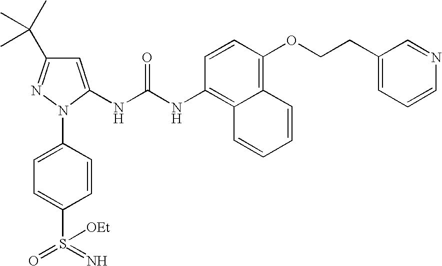

### Example 143A

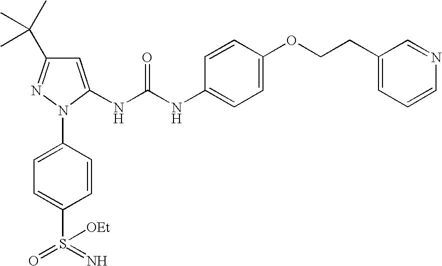

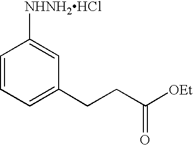

### Example Y

To a solution of 3-nitro-benzaldehyde (15.1 g, 0.1 mol) in CH₂Cl₂ (200 mL) was added (triphenyl-15-phosphanylidene)-acetic acid ethyl ester (34.8 g, 0.1 mol) in CH₂Cl₂ (100 mL) dropwise at 0° C., which was stirred for 2 h. After removal the solvent under reduced pressure, the residue was purified by column chromatography to afford 3-(3-nitro-phenyl)-acrylic acid ethyl ester (16.5 g, 74.6%) ¹H-NMR (400 MHz, CDCl₃): 8.42 (s, 1H), 8.23 (d, J=8.0 Hz, 1H), 7.82 (d, J=7.6 Hz, 1H), 7.72 (d, J=16.0 Hz, 1H), 7.58 (t, J=8.0 Hz, 1H), 6.56 (d, J=16.0 Hz, 1H), 4.29 (q, J=7.2 Hz, 2H), 1.36 (t, J=6.8 Hz, 3H).

A mixture of 3-(3-nitro-phenyl)-acrylic acid ethyl ester (16.5 g, 74.6 mmol) and Pd/C (1.65 g) in methanol (200 mL) was stirred under 40 psi of H₂ at RT for 2 h then filtered over celite. After removal the solvent, 14 g of 3-(3-amino-phenyl)-propionic acid ethyl ester was obtained and used directly without further purification. ¹H-NMR (400 MHz, CDCl₃): 7.11 (t, J=5.6 Hz, 1H), 6.67 (d, J=7.2 Hz, 1H), 6.63-6.61 (m, 2H), 4.13 (q, J=7.2 Hz, 2H), 2.87 (t, J=8.0 Hz, 2H), 2.59 (t, J=7.6 Hz, 2H), 1.34 (t, J=6.8 Hz, 3H).

To a solution of 3-(3-amino-phenyl)-propionic acid ethyl ester (14 g, 72.5 mmol) in concentrated HCl (200 mL) was added an aqueous solution (10 mL) of NaNO₂ (5 g, 72.5 mmol) at 0° C. and the resulting mixture was stirred for 1 h. A solution of SnCl₂.2H₂O (33 g, 145 mmol) in concentrated HCl (150 mL) was then added at 0° C. The reaction solution was stirred for an additional 2 h at RT. The precipitate was filtered and washed with ethanol and ether to give 3-(3-hydrazino-phenyl)-propionic acid ethyl ester as a white solid, which was used without further purification.

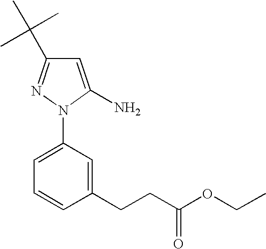

### Example Z

A mixture of Example Y (13 g, 53.3 mmol) and 4,4-dimethyl-3-oxo-pentanenitrile (6.9 g, 55 mol) in ethanol (150 mL) was heated to reflux overnight. The reaction solution was evaporated under reduced pressure. The residue was purified by column chromatography to give 3-[3-(5-amino-3-t-butyl-pyrazol-1-yl)-phenyl]-propionic acid ethyl ester (14.3 g, 45.4 mmol) as a white solid. ¹H NMR (DMSO-d₆): 7.39-7.32 (m, 3H), 7.11 (d, J=6.8 Hz, 1H), 5.34 (s, 1H), 5.16 (s, 2H), 4.03 (q, J=7.2 Hz, 2H), 2.88 (t, J=7.6 Hz, 2H), 2.63 (t, J=7.6 Hz, 2H), 1.19 (s, 9H), 1.15 (t, J=7.2 Hz, 3H).

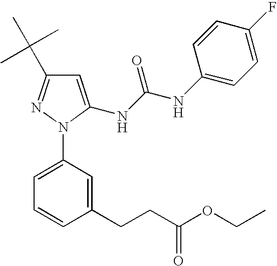

### Example 145

A solution of 4-fluoro-phenylamine (111 mg, 1.0-mmol) and CDI (165 mg, 1.0 mmol) in DMF (2 mL) was stirred at RT for 30 min, and was then added to a solution of Example Z (315 mg, 1.0 mmol) in DMF (2 mL). The resulting mixture was stirred at RT overnight then added to water (50 mL). The reaction mixture was extracted with ethyl acetate (3×50 mL) and the combined organic extracts were washed with brine, dried (NaSO₄) and filtered. After concentrated under reduced pressure, the residue was purified by flash chromatography to afford 3-(3-{3-t-butyl-5-[3-(4-fluoro-phenyl)-ureido]-pyrazol-1-yl}-phenyl)-propionic acid ethyl ester (150 mg, 33%). ¹H-NMR (CDCl₃): 7.91 (s, 1H), 7.42 (d, J=4.8 Hz, 1H), 7.37-7.34 (m, 2H), 7.28 (s, 1H), 7.17-7.16 (m, 2H), 6.98 (t, J=8.8 Hz, 2H), 6.59 (s, 1H), 4.04 (q, J=7.2 Hz, 2H), 3.03 (t, J=7.2 Hz, 2H), 2.77 (t, J=7.2 Hz, 2H), 1.36 (s, 9H), 1.17 (t, J=7.2 Hz, 3H); MS (ESI) m/z: 453 (M+H⁺).

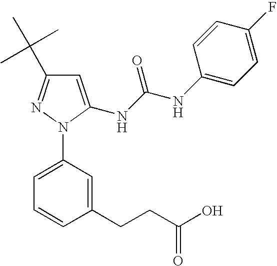

### Example 146

A solution of Example 145 (45 mg, 0.1 mmol) and 2N LiOH (3 mL) in MeOH (3 mL) was stirred at RT overnight. The reaction mixture was neutralized to pH=4, extracted with ethyl acetate (3×20 mL), the combined organic extracts were washed with brine, dried (NaSO₄) and filtered. The filtrate was concentrated to afford 3-(3-{3-t-butyl-5-[3-(4-fluoro-phenyl)-ureido]-pyrazol-1-yl}-phenyl)-propionic acid, (37 mg, 90%). ¹H NMR (CD₃OD): 7.63-7.62 (m, 2H), 7.56 (s, 1H), 7.53-7.48 (m, 1H), 7.41-7.38 (m, 2H), 7.04 (t, J=8.8 Hz, 2H), 5.49 (s, 1H), 3.07 (t, J=7.6 Hz, 2H), 2.72 (t, J=7.6 Hz, 2H), 1.42 (s, 9H); MS (ESI) m/z: 415 (M+H⁺).

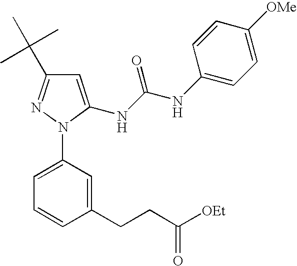

### Example 147

A mixture of 4-methoxy-phenylamine (123 mg, 1.0 mmol) and CDI (165 mg, 1.0 mmol) in DMF (2 mL) was stirred at RT for 30 min, and was then added a solution of Example Z (315 mg, 1.0 mmol) in DMF (2 mL). The resulting mixture was stirred at RT overnight then quenched with of water (50 mL). The reaction mixture was extracted with ethyl acetate (3×50 mL) and the combined organic extracts were washed with brine, dried (NaSO₄), filtered, concentrated under reduced presume to yield a residue which was purified by flash chromatography to afford 3-(3-{3-t-butyl-5-[3-(4-methoxy-phenyl)-ureido]-pyrazol-1-yl}-phenyl)-propionic acid ethyl ester (210 mg, 45%). ¹H-NMR (CD₃OD): 7.46 (t, J=7.6 Hz, 1H), 7.38 (s, 1H), 7.34 (d, J=7.6 Hz, 2H), 7.24 (d, J=8.4 Hz, 2H), 6.84 (d, J=8.4 Hz, 2H), 6.38 (s, 1H), 4.09 (q, J=7.2 Hz, 2H), 3.75 (s, 3H), 3.00 (t, J=7.6 Hz, 2H), 2.68 (t, J=7.6 Hz, 2H), 1.33 (s, 9H), 1.20 (t, J=7.6 Hz, 3H); MS (ESI) m/z: 465 (M+H⁺).

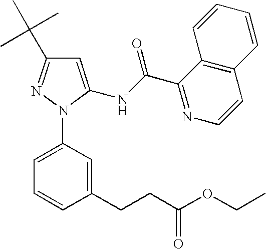

### Example 149

A solution of isoquinoline-1-carboxylic acid (346 mg, 2.0 mmol), Example Z (315 mg, 1.0 mmol), EDCI (394 mg, 2.0 mmol), HOBt (270 mg, 2.0 mmol), and NMM (1.0 mL) in DMF (10 mL) was stirred at RT overnight. After quenching with water (100 mL), the reaction mixture was extracted with ethyl acetate (3×100 mL). The combined organic extracts were washed with brine, dried (NaSO₄), filtered and concentrated under reduced pressure to yield a residue which was purified by flash chromatography to afford 3-(3-{3-t-butyl-5-[(isoquinoline-1-carbonyl)-amino]-pyrazol-1-yl}-phenyl)-propionic acid ethyl ester, (380 mg, 80%). ¹H-NMR (DMSO-d₆): 8.83 (d, J=8.4 Hz, 1H), 8.85 (d, J=5.2 Hz, 1H), 8.09 (s, 1H), 8.07 (d, J=8.4 Hz, 1H), 7.82 (t, J=8.0 Hz, 1H), 7.72 (t, J=8.0 Hz, 1H), 7.52 (s, 1H), 7.44 (d, J=8.0 Hz, 1H), 7.39 (t, J=5.2 Hz, 1H), 7.22 (d, J=8.0 Hz, 1H), 6.57 (s, 1H), 3.98 (q, J=7.2 Hz, 2H), 2.84 (t, J=7.6 Hz, 2H), 2.57 (t, J=7.6 Hz, 2H), 1.32 (s, 9H), 1.10 (t, J=7.6 Hz, 1H); MS (ESI) m/z: 471 (M+H⁺).

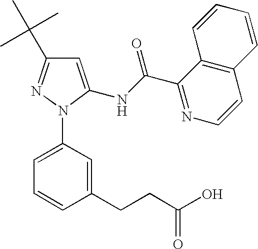

### Example 150

A solution of Example 149 (47 mg, 0.1 mmol) and 2N LiOH (3 mL) in MeOH (3 mL) was stirred at RT overnight. The reaction mixture was neutralized to pH=4, extracted with ethyl acetate (3×20 mL), and the combined organic extracts were washed with brine, dried (NaSO₄) and filtered. The filtrate was concentrated to afford 3-(3-{3-t-butyl-5-[(isoquinoline-1-carbonyl)-amino]-pyrazol-1-yl}-phenyl)-propionic acid, (39 mg, 87%). ¹H-NMR (DMSO-d₆): 10.77 (s, 1H), 9.68 (d, J=7.6 Hz, 1H), 8.44 (d, J=5.2 Hz, 1H), 7.89-7.44 (m, 2H), 7.78-7.74 (m, 2H), 7.49-7.47 (m, 3H), 7.30-7.27 (m, 3H), 6.95 (s, 1H), 3.05 (t, J=7.2 Hz, 2H), 2.75 (t, J=7.6 Hz, 2H), 1.42 (s, 9H); MS (ESI) m/z: 443 (M+H⁺).

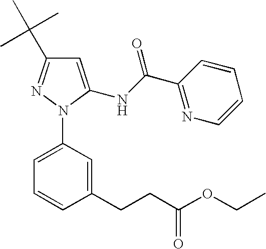

### Example 151

A solution of pyridine-2-carboxylic acid (246 mg, 2.0 mmol), Example Z (315 mg, 1.0 mmol), EDCI (394 mg, 2.0 mmol), HOBt (270 mg, 2.0 mmol), NMM (1.0 mL) in DMF (10 mL) was stirred at RT overnight. After quenching with water (100 mL), the reaction mixture was extracted with ethyl acetate (3×100 mL). The combined organic extracts were washed with brine, dried (NaSO₄), filtered and concentrated under reduced pressure to yield a residue which was purified by flash chromatography to afford 3-(3-{3-t-butyl-5-[(pyridine-2-carbonyl)-amino]-pyrazol-1-yl}-phenyl)-propionic acid ethyl ester (300 mg, 70%). ¹H-NMR (CDCL₃): 8.53 (d, J=4.4 Hz, 1H), 8.26 (d, J=7.2 Hz, 1H), 7.90 (t, J=8.0 Hz, 1H), 7.48-7.43 (m, 4H), 7.27 (s, 1H), 6.87 (s, 1H), 4.13 (q, J=7.2 Hz, 2H), 3.04 (t, J=7.6 Hz, 2H), 2.71 (t, J=7.6 Hz, 2H), 1.39 (s, 9H), 1.24 (t, J=7.2 Hz, 3H); MS (ESI) m/z: 421 (M+H⁺).

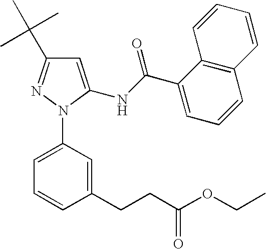

### Example 152

A solution of Example Z (315 mg, 1.0 mmol) and Barton's base (0.5 mL) in anhydrous CH₂Cl₂ (5 mL) under N₂ was stirred at RT for 30 min, and then added to a solution of naphthalene-1-carbonyl fluoride (348 mg, 0.2 mmol) in anhydrous CH₂Cl₂ (5 mL). The resulting mixture was stirred at RT overnight. After quenching with water (100 mL), the reaction mixture was extracted with ethyl acetate (3×100 mL). The combined organic extracts were washed with brine, dried (NaSO₄), filtered and concentrated under reduced pressure to yield a residue which was purified by flash chromatography to afford 3-(3-{3-t-butyl-5-[(naphthalene-1-carbonyl)-amino]-pyrazol-1-yl}-phenyl)-propionic acid ethyl ester, (350 mg, 74%). ¹H-NMR (CDCL₃): 8.29 (d, J=8.0 Hz, 1H), 7.98 (d, J=7.2 Hz, 2H), 7.89 (d, J=7.2 Hz, 1H), 7.62-7.57 (m, 3H), 7.49-7.28 (m, 4H), 7.03 (s, 1H), 3.94 (q, J=7.2 Hz, 2H), 2.96 (t, J=7.2 Hz, 2H), 2.58 (t, J=7.2 Hz, 2H), 1.45 (s, 9H), 1.13 (t, J=7.2 Hz, 3H); MS (ESI) m/z: 470 (M+H⁺).

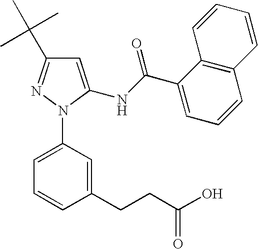

### Example 153

A solution of Example 152 (47 mg, 0.1 mmol) and 2N LiOH (3 mL) in MeOH (3 mL) was stirred at RT overnight. The reaction mixture was neutralized to pH=4, and extracted with ethyl acetate (3×20 mL). The combined organic extracts were washed with brine, and dried (NaSO₄) and filtered. The filtrate was concentrated to afford 3-(3-{3-t-butyl-5-[(isoquinoline-1-carbonyl)-amino]-pyrazol-1-yl}-phenyl)-propionic acid, (38 mg, 86%). ¹H NMR (DMSO-d₆): 7.99 (d, J=8.0 Hz, 1H), 7.90 m, 2H), 7.62 (m, 1H), 7.54-7.42 (m, 6H), 7.35 (m, 1H), 6.54 (s, 1H), 2.94 (t, J=7.6 Hz, 2H), 2.57 (t, J=7.2 Hz, 2H), 1.38 (s, 9H); MS (ESI) m/z: 443 (M+H⁺).

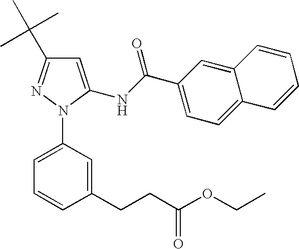

### Example 154

A solution of naphthalene-2-carboxylic acid (344 mg, 2.0 mmol) in SOCl₂ (10 mL) was heated to reflux for 2 h. After concentration under reduced pressure, the residue was dissolved into CH₂Cl₂ (5 mL) and was dropped into a solution of Example Z (315 mg, 1.0 mmol) in CH₂Cl₂ (10 mL) at 0° C., and was then stirred at RT overnight. After quenching with water (50 mL), the reaction mixture was extracted with CH₂Cl₂ (3×100 mL). The combined organic extracts were washed with brine, dried (NaSO₄), filtered and concentrated under reduced pressure to yield a residue which was purified by flash chromatography to afford 3-(3-{3-t-butyl-5-[(naphthalene-2-carbonyl)-amino]-pyrazol-1-yl}-phenyl)-propionic acid ethyl ester (180 mg, 38%). ¹H-NMR (CDCL₃): 8.24 (s, 1H), 8.21 (s, 1H), 7.91 (d, J=8.4 Hz, 2H), 7.88 (d, J=8.4 Hz, 1H), 7.73 (d, J=8.4 Hz, 1H), 7.63-7.49 (m, 3H), 7.45-7.26 (m, 3H), 6.94 (s, 1H), 4.02 (q, J=7.2 Hz, 2H), 3.04 (t, J=7.6 Hz, 2H), 2.67 (t, J=7.6 Hz, 2H), 1.43 (s, 9H), 1.17 (t, J=7.2 Hz, 3H); MS (ESI) m/z: 470 (M+H⁺).

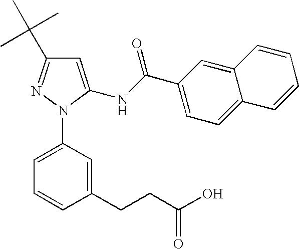

### Example 155

A solution of Example 154 (47 mg, 0.1 mmol) and 2N LiOH (3 mL) in MeOH (3 mL) was stirred at RT overnight. The reaction mixture was neutralized to pH=4, and extracted with ethyl acetate (3×20 mL). The combined organic extracts were washed with brine, and dried (NaSO₄) and filtered. The filtrate was concentrated to afford 3-(3-{3-t-butyl-5-[(isoquinoline-2-carbonyl)-amino]-pyrazol-1-yl}-phenyl)-propionic acid, (37 mg, 84%). ¹H-NMR (CDCl₃): 8.25 (s, 1H), 8.18 (s, 1H), 7.91-7.86 (m, 3H), 7.75 (d, J=8.0 Hz, 1H), 7.59-7.55 (m, 2H), 7.48-7.39 (m, 3H), 7.28 (s, 1H), 6.81 (s, 1H), 3.02 (t, J=7.6 Hz, 2H), 2.69 (t, J=7.6 Hz, 2H), 1.42 (s, 9H); MS (ESI) m/z: 442 (M+H⁺).

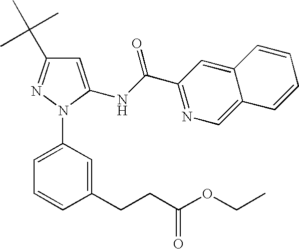

### Example 156

A solution of isoquinoline-3-carboxylic acid (346 mg, 2.0 mmol), Example Z (315 mg, 1.0 mmol), EDCI (394 mg, 2.0 mmol), HOBt (270 mg, 2.0 mmol), and NMM (1.0 mL) in DMF (10 mL) was stirred at RT overnight. After quenching with water (50 mL), the reaction mixture was extracted with ethyl acetate (3×100 mL). The combined organic extracts were washed with brine, dried (NaSO₄) and filtered. After concentrated under reduced pressure, the residue was purified by flash chromatography to afford 3-(3-{3-t-butyl-5-[(isoquinoline-3-carbonyl)-amino]-pyrazol-1-yl}-phenyl)-propionic acid ethyl ester (250 mg, 54%). ¹H-NMR (CD₃OD): 9.24 (s, 1H), 8.63 (s, 1H), 8.17 (d, J=8.0 Hz, 1H), 8.11 (d, J=8.0 Hz, 1H), 7.88 (t, J=7.6 Hz, 1H), 7.81 (t, J=7.6 Hz, 1H), 7.50 (s, 1H), 7.48 (d, J=7.6 Hz, 1H), 7.54 (d, J=7.6 Hz, 2H), 7.36 (d, J=7.6 Hz, 1H), 6.75 (s, 1H), 4.04 (q, J=7.6 Hz, 2H), 3.01 (t, J=7.6 Hz, 2H), 2.69 (t, J=7.6 Hz, 2H), 1.39 (s, 9H), 1.14 (t, J=7.6 Hz, 3H); MS (ESI) m/z: 471 (M+H⁺).

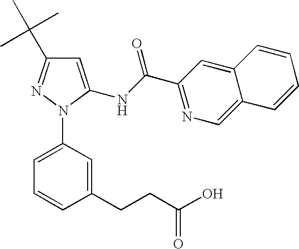

### Example 157

A solution of Example 156 (47 mg, 0.1 mmol) and 2N LiOH (3 mL) in MeOH (3 mL) was stirred at RT overnight. The reaction mixture was neutralized to pH=4, and extracted with ethyl acetate (3×20 mL). The combined organic extracts were washed with brine, and dried (NaSO₄) and filtered. The filtrate was concentrated to afford 3-(3-{3-t-butyl-5-[(isoquinoline-3-carbonyl)-amino]-pyrazol-1-yl}-phenyl)-propionic acid, (39 mg, 88%). ¹H NMR (CDCL3): 10.49 (s, 1H), 9.16 (s, 1H), 8.69 (s, 1H), 8.03 (d, J=7.6 Hz, 2H), 7.81 (t, J=7.2 Hz, 1H), 7.73 (t, J=7.2 Hz, 1H), 7.48-7.39 (m, 3H), 7.28 (br s, 1H), 6.94 (s, 1H), 3.02 (t, J=7.6 Hz, 2H), 2.79 (t, J=7.6 Hz, 2H), 1.42 (s, 9H); MS (ESI) m/z: 442 (M+H⁺).

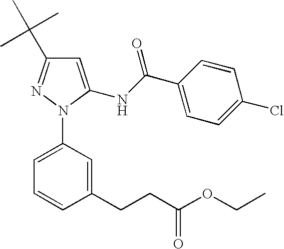

### Example 158

A solution of 4-chlorobenzoic acid (312 mg, 2.0 mmol) in SOCl₂ (10 mL) was heated to reflux for 2 h. After removal of the solvent, the residue was dissolved into CH₂Cl₂ (5 mL) and was dropped into a solution of Example Z (315 mg, 1.0 mmol) in CH₂Cl₂ (10 mL) at 0° C., was then stirred at RT overnight. After quenching with water (50 mL), the reaction mixture was extracted with CH₂Cl₂ (3×100 mL). The combined organic extracts were washed with brine, dried (NaSO₄) and filtered. After concentrated under reduced pressure, the residue was purified by flash chromatography to afford 3-{3-[3-t-butyl-5-(4-chloro-benzoylamino)-pyrazol-1-yl]-phenyl}-propionic acid ethyl ester (290 mg, 64%). ¹H-NMR (CDCL₃); 8.02 (s, 1H), 7.67 (d, J=8.4 Hz, 2H), 7.46 (t, J=7.6 Hz, 1H), 7.44 (d, J=8.4 Hz, 2H), 7.36 (t, J=8.4 Hz, 3H), 6.87 (s, 1H), 4.06 (q, J=7.6 Hz, 2H), 3.02 (t, J=7.6 Hz, 2H), 2.67 (t, J=7.6 Hz, 2H), 1.40 (s, 9H), 1.12 (t, J=7.6 Hz, 3H); MS (ESI) m/z: 454 (M+H⁺).

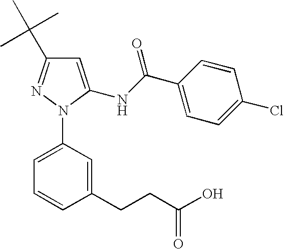

### Example 159

A solution of Example 158 (45 mg, 0.1 mmol) and 2N LiOH (3 mL) in MeOH (3 mL) was stirred at RT overnight. The reaction mixture was neutralized to pH=4, and extracted with ethyl acetate (3×20 mL). The combined organic extracts were washed with brine, and dried (NaSO₄) and filtered. The filtrate was concentrated to afford 3-{3-[3-t-butyl-5-(4-chloro-benzoylamino)-pyrazol-1-yl]-phenyl}-propionic acid, (38.5 mg, 87%). ¹H NMR (DMSO-d₆): 10.38 (s, 1H), 7.85 (d, J=8.4 Hz, 1H), 7.56 (d, J=8.4 Hz, 2H), 7.39 (s, 1H), 7.32 (d, J=4.8 Hz, 2H), 7.15 (t, J=4.8 Hz, 1H), 6.38 (s, 1H), 2.80 (t, J=7.6 Hz, 2H), 2.44 (t, J=7.2 Hz, 2H), 1.29 (s, 9H); MS (ESI) m/z: 426 (M+H).

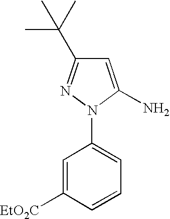

### Example AA

To a solution of m-aminobenzoic acid (200.0 g, 1.46 mmol) in concentrated HCl (200 mL) was added an aqueous solution (250 mL) of NaNO₂ (102 g, 1.46 mmol) at 0° C. and the reaction mixture was stirred for 1 h. A solution of SnCl₂.2H₂O (662 g, 2.92 mmol) in concentrated HCl (2000 mL) was then added at 0° C. The reaction solution was stirred for an additional 2 h at RT. The precipitate was filtered and washed with ethanol and ether to give 3-hydrazino-benzoic acid hydrochloride as a white solid, which was used for the next reaction without further purification. ¹H NMR (DMSO-d₆): 10.85 (s, 3H), 8.46 (s, 1H), 7.53 (s, 1H), 7.48 (d, J=7.6 Hz, 1H), 7.37 (m, J=7.6 Hz, 1H), 7.21 (d, J=7.6 Hz, 1H).

A mixture of 3-hydrazino-benzoic acid hydrochloride (200 g, 1.06 mol) and 4,4-dimethyl-3-oxo-pentanenitrile (146 g, 1.167 mol) in ethanol (2 L) was heated to reflux overnight. The reaction solution was evaporated under reduced pressure. The residue was purified by column chromatography to give 3-(5-amino-3-t-butyl-pyrazol-1-yl)-benzoic acid ethyl ester (116 g, 40%) as a white solid together with 3-(5-amino-3-t-butyl-pyrazol-1-yl)-benzoic acid (93 g, 36%). 3-(5-amino-3-t-butyl-pyrazol-1-yl)-benzoic acid and ethyl ester: ¹H NMR (DMSO-d₆): 8.09 (s, 1H), 8.05 (brd, J=8.0 Hz, 1H), 7.87 (br d, J=8.0 Hz, 1H), 7.71 (t, J=8.0 Hz, 1H), 5.64 (s, 1H), 4.35 (q, J=7.2 Hz, 2H), 1.34 (t, J=7.2 Hz, 3H), 1.28 (s, 9H).

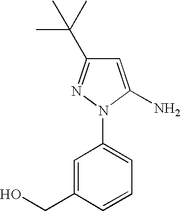

### Example BB

To a solution of Example AA (19.5 g, 68.0 mmol) in THF (200 mL) was added LiAlH₄ powder (5.30 g, 0.136 mol) at −10° C. under N₂. The mixture was stirred for 2 h at RT and excess LiAlH₄ was destroyed by slow addition of ice. The reaction mixture was acidified to pH=7 with diluted HCl, the solution concentrated under reduced pressure, and the residue was extracted with ethyl acetate. The combined organic extracts were concentrated to give [3-(5-amino-3-t-butyl-pyrazol-1-yl)-phenyl]-methanol (16.35 g, 98%) as a white powder. ¹H NMR (DMSO-d₆): 9.19 (s, 1H), 9.04 (s, 1H), 8.80 (s, 1H), 8.26-7.35 (m, 1H), 6.41 (s, 1H), 4.60 (s, 2H), 1.28 (s, 9H); MS (ESI) m/z: 415 (M+H⁺).

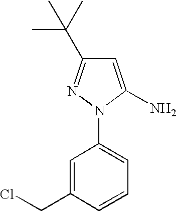

### Example CC

A solution of Example BB (13.8 g, 56.00 mmol) and SOCl₂ (8.27 mL, 0.11 mol) in THF (200 mL) was refluxed for 3 h and concentrated under reduced pressure to yield 5-t-butyl-2-(3-chloromethyl-phenyl)-2H-pyrazol-3-ylamine (14.5 g, 98%) as white powder which was used without further purification. ¹H NMR (DMSO-d6), 87.62 (s, 1H), 7.53 (d, J=8.0 Hz, 1H), 7.43 (t, J=8.0 Hz, 1H), 7.31 (d, J=7.2 Hz, 1H), 5.38 (s, 1H), 5.23 (br s, 2H), 4.80 (s, 2H), 1.19 (s, 9H). MS (ESI) m/z: 264 (M+H⁺).

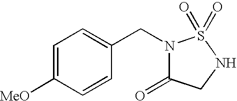

### Example DD

To a stirred solution of chlorosulfonyl isocyanate (1.43 g, 10.0 mmol) in CH₂Cl₂ (20 mL) at 0° C. was added 2-methyl-propan-2-ol (0.74 g, 10.0 mmol) at such a rate that the reaction solution temperature did not rise above 5° C. After being stirred for 1.5 h, a solution of glycine ethyl ester (1.45 g, 12.0 mmol) and Et₃N (3.2 mL, 25.0 mmol) in CH₂Cl₂ (20 mL) was added at such a rate that the reaction temperature didn't rise above 5° C. When the addition was completed, the solution was waited to RT and stirred overnight. The reaction mixture was poured into 10% HCl and extracted with CH₂Cl₂. The organic layer was washed with saturated NaCl, dried (Mg₂SO₄) and filtered. After removal of the solvent, the crude product was washed with CH₂Cl₂ to afford ethyl 2-((N-(butyloxycarbonyl)sulfamoyl)amino)acetate (2.4 g, 85%). ¹H-NMR (DMSO): δ 10.85 (s, 1H), 8.04 (t, J=6.0 Hz, 1H), 4.07 (q, J=5.6 Hz, 2H), 3.77 (d, J=6.0 Hz, 2H), 1.40 (s, 9H), 1.18 (t, J=7.2 Hz, 3H).

To a solution of (4-methoxyphenyl)-methanol (1.4 g, 8.5 mmol) and triphenyl-phosphane (2.6 g, 8.5 mol) in dry THF was added a solution of ethyl 2-((N-(butyloxycarbonyl)sulfamoyl)amino)acetate from the previous step (2.4 g, 8.5 mol) and DIAD (2.0 g, 8.5 mmol) in dry THF dropwise at 0° C. under N₂ atmosphere. The mixture was stirred at 0° C. for 2 h, warned to RT and stirred overnight. After the solvent was removed in vacuo, the residue was purified by column chromatography to afford ethyl 2-((N-(butyloxycarbonyl)-N-(p-methoxybenzyl)sulfamoyl)amino)acetate (2.3 g, 69%) as a white solid. ¹H-NMR (CDCl₃): δ 7.32 (d, J=8.8 Hz, 2H), 6.85 (d, J=8.8 Hz, 2H), 5.71 (m, 1H), 4.76 (s, 2H), 4.14 (q, J=7.2 Hz, 2H), 3.80 (s, 3H), 3.55 (d, J=5.2 Hz, 2H), 1.54 (s, 9H), 1.25 (t, J=7.2 Hz, 3H).

To a solution of HCl in methanol (2 M) was added ethyl 2-((N-(butyloxycarbonyl)-N-(p-methoxybenzyl)sulfamoyl)amino)acetate from the previous step (2.0 g, 5.0 mmol) in portions at RT and the mixture was stirred for 3 h. After the solvent was removed in vacuo, the residue was washed with diethyl ether to afford ethyl 2-((N-(p-methoxybenzyl)sulfamoyl)amino)acetate (1.0 g, 70%). ¹H-NMR (DMSO-d₆): δ 7.43 (t, J=6.0 Hz, 1H), 7.287 (t, J=6.4 Hz, 1H), 7.21 (d, J=8.4 Hz, 2H), 6.86 (d, J=8.4 Hz, 2H), 3.94 (d, J=4.8 Hz, 2H), 3.71 (s, 3H), 3.64 (d, J=6.0 Hz, 2H), 3.62 (s, 3H).

To a solution of ethyl 2-((N-(p-methoxybenzyl)sulfamoyl)amino)acetate from the previous step (1.0 g, 3.47 mmol) in DMF (50 mL) was added KO-t-Bu (1.56 g, 13.88 mmol) in portions under N₂ atmosphere at RT. The mixture was stirred overnight then quenched with HCl/methanol (2 M). After the solvent was removed in vacuo, the residue was washed with water to afford 2-(4-methoxy-benzyl)-1,1-dioxo-1λ⁶-[1,2,5]thiadiazolidin-3-one (480 mg, 54%). ¹H-NMR (CDCl₃): δ 7.36 (d, J=8.4 Hz, 2H), 6.87 (d, J=8.8 Hz, 2H), 4.87 (m, 1H), 4.68 (s, 2H), 4.03 (d, J=7.2 Hz, 2H), 3.80 (s, 3H).

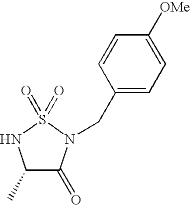

### Example EE

To a stirred solution of chlorosulfonyl isocyanate (1.43 g, 10.0 mmol) in CH₂Cl₂ (20 mL) at 0° C. was added benzyl alcohol (1.08 g, 10.0 mmol) at such a rate that the reaction solution temperature did not rise above 5° C. After stirring for 1.5 h, a solution of L-alanine methyl ester (1.45 g, 12.0 mmol) and Et₃N (3.2 mL, 25.0 mmol) in CH₂Cl₂ (20 mL) was added at such a rate that the reaction temperature didn't rise above 5° C. When the addition was completed, the reaction solution was allowed to warm up to RT and stirred overnight. The reaction mixture was poured into 10% HCl, extracted with CH₂Cl₂, the organic extracts washed with saturated NaCl, dried (Mg₂SO₄), and filtered. After removal of the solvent, the crude product was recrystallized in PE/EA (10:1) to afford the desired product (2.5 g, 79%), which was used directly in the next step. ¹H-NMR (DMSO): δ 11.31 (s, 1H), 8.43 (d, J=8.0 Hz, 1H), 7.37-7.32 (m, 5H), 5.11 (s, 2H), 4.03 (m, 1H), 3.57 (s, 3H), 1.23 (d, J=7.2 Hz, 3H).

A mixture of material from the previous reaction (2.5 g, 12 mmol) and Pd/C (10%, 250 mg) in methanol was stirred for 4 h at 50° C. under H₂ atmosphere (55 psi). After the catalyst was removed by suction, the filtrate was evaporated to afford the desired compound (1.37 g, 92%) as a white solid, which was used directly in the next step. ¹H-NMR (CDCl₃): δ 5.51 (d, J=5.6 Hz, 1H), 4.94 (br, 2H), 4.18 (m, 1H), 3.78 (s, 3H), 1.46 (d, J=7.2 Hz, 3H).

To a solution of 2.0 N of NaOMe in methanol (20 mL) was added a solution of compound form the previous reaction (1.2 g, 6.1 mmol) in methanol and the resulting mixture was heated to reflux overnight. After cooling down, a solution of HCl in methanol was added to acidify to pH 7. The resulted salt was filtered off and the filtrate was evaporated to dryness to afford a light yellow solid which was used directly in the next step (600 mg, 66%). ¹H-NMR (DMSO-d₆): δ 6.04 (d, J=4.8 Hz, 1H), 3.60 (m, 1H), 1.11 (d, J=7.2 Hz, 3H):

A mixture of compound from the previous step (500 mg, 3.33 mmol) and 1-chloromethyl-4-methoxybenzene (156 mg, 1.0 mmol) in acetonitrile was heated to reflux overnight together with K₂CO₃ (207 mg, 1.5 mmol) and KI (250 mg, 1.5 mmol) under N₂ atmosphere. After cooling, the salt was filtered off and the filtrate was purified by column to afford 2-(4-methoxybenzyl)-(S)-4-methyl-1,1-dioxo-1λ⁶-[1,2,5]thiadiazolidin-3-one as a white solid (200 mg), which was used without further purification.

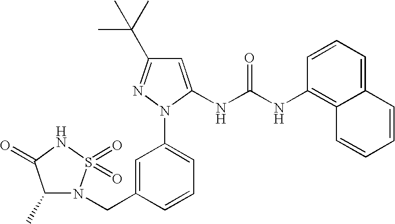

### Example 160

To a solution of Example EE (100 mg, 0.37 mmol) in anhydrous DMF (3 mL) was added NaH (18 mg, 0.44 mmol) at 0° C. After stirring for 0.5 h at 0° C., a solution of Example E (160 mg, 0.37 mmol) in anhydrous DMF (3 mL) was added to the reaction mixture, which was stirred overnight at RT and subsequently concentrated under reduced pressure to yield a crude solid which was used without further purification.

A solution of the crude material from the previous reaction (60 mg, 0.090 mmol) in trifluoroacetic acid (3 mL) was stirred at 50° C. for 4 h. After the solvent was removed, the residue was purified by preparative HPLC to afford 1-{5-t-butyl-2-[3-((S)-3-methyl-1,1,4-trioxo-1λ⁶-[1,2,5]thiadiazolidin-2-ylmethyl)-phenyl]-2H-pyrazol-3-yl}-3-naphthal-en-1-yl-urea as white power (45 mg). ¹H NMR (DMSO-d₆): 9.04 (s, 1H), 8.87 (s, 1H), 8.02 (d, J=8.0 Hz, 1H), 7.89 (d, J=7.2 Hz, 2H), 7.62 (d, J=8.0 Hz, 2H), 7.41-7.52 (m, 6H), 6.40 (s, 1H), 4.31-4.49 (dd, J=8.0 Hz, 2H), 4.03 (q, J=6.8 Hz, 1H), 1.27 (s, 9H), 1.17 (d, J=8.0 Hz, 3H). MS (ESI) m/z: 547 (M+H⁺).

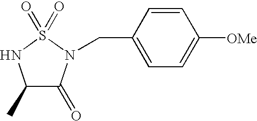

### Example FF

2-(4-methoxy-benzyl)-(R)-4-methyl-1,1-dioxo-1λ⁶-[1,2,5]thiadiazolidin-3-one was prepared from D-alanine ethyl ester using the same procedure as Example EE.

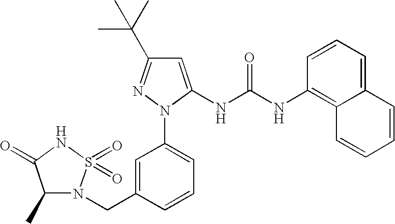

### Example 161

To a solution of Example FF (60 mg, 0.22 mmol) in anhydrous DMF (2 mL) was added NaH (11 mg, 0.27 mmol) at 0° C. After stirring for 0.5 h at 0° C., a solution of Example D (100 mg, 0.22 mmol) in anhydrous DMF (2 mL) was added to the reaction mixture, which was stirred overnight at RT. The crude reaction mixture was concentrated under reduced pressure and the residue by purified through preparative HPLC to yield 1-(5-t-butyl-2-{3-[5-(4-methoxy-benzyl)-(R)-3-methyl-1,1,4-trioxo-1λ⁶-[1,2,5]-thiadiazolidin-2-ylmethyl]-phenyl}-2H-pyrazol-3-yl)-3-naphthalene-1-yl-urea (20 mg). ¹H NMR (DMSO-d₆): 8.98 (s, 1H), 8.81 (s, 1H), 8.00 (d, J=8.0 Hz, 1H), 7.90 (d, J=7.2 Hz, 2H), 7.62 (s, 2H), 7.51-7.55 (m, 6H), 7.44 (d, J=7.6 Hz, 2H), 7.22 (d, J=8.8 Hz, 2H), 6.86 (d, J=8.8 Hz, 2H), 6.40 (s, 1H), 4.57-4.62 (dd, J=8.0 Hz, 4H), 4.53 (q, J=7.6 Hz, 1H), 3.71 (s, 3H), 1.30 (d, J=8.0 Hz, 3H), 1.27 (s, 9H). MS (ESI) m/z: 653 (M+H⁺).

A solution of 1-(5-t-Butyl-2-{3-[5-(4-methoxy-benzyl)-(R)-3-methyl-1,1,4-trioxo-1λ⁶-[1,2,5]-thiadiazolidin-2-ylmethyl]-phenyl}-2H-pyrazol-3-yl)-3-naphthalen-1-yl-urea (20 mg, 0.030 mmol) in trifluoroacetic acid (2 mL) was stirred at 50° C. for 4 h. After the solvent was removed, the residue was purified by preparative-HPLC to afford 1-{5-t-butyl-2-[3-((R)-3-methyl-1,1,4-trioxo-1λ⁶-[1,2,5]thiadiazolidin-2-ylmethyl)-phenyl]-2H-pyrazol-3-yl}-3-naphthalen-1-yl-urea as a white power (6 mg). ¹H NMR (DMSO-d₆): 8.99 (s, 1H), 8.80 (s, 1H), 8.00 (d. J=7.2 Hz, 1H), 7.90 (d, J=7.2 Hz, 2H), 7.60-7.64 (m, 2H), 7.44-7.54 (m, 7H), 6.41 (s, 1H), 4.31-4.49 (dd, J=8.0 Hz, 2H), 4.03 (q, J=7.6 Hz, 1H), 1.27 (s, 9H), 1.19 (d, J=8.0 Hz, 3H). MS (ESI) m/z: 533 (M+H⁺).

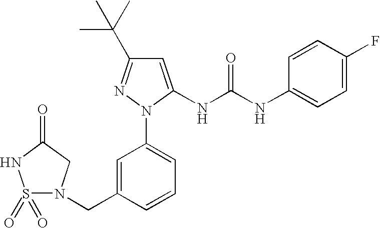

### Example 162

To a solution of Example CC (0.263 g, 1.0 mmol) in THF (2.0 mL) was added a solution of 1-fluoro-4-isocyanato-benzene (0.114 mL, 1.10 mmol) in THF (5.0 mL) at 0° C. The mixture was stirred at RT for 1 h then heated until all solids were dissolved. The mixture was stirred at RT for 3 h and poured into water (20 mL). The resulting precipitate was filtered, washed with diluted HCl and H₂O, dried under reduced pressure to yield 1-[5-t-butyl-2-(3-chloromethyl-phenyl)-2H-pyrazol-3-yl]-3-(4-fluoro-phenyl)-urea (400 mg) as a white power. ¹H NMR (DMSO-d₆): 8.99 (s, 1H), 8.38 (s, 1H), 7.59 (s, 1H), 7.44-7.51 (m, 3H), 7.38-7.40 (m, 2H), 7.08 (t, J=8.8 Hz, 2H), 6.34 (s, 1H), 4.83 (s, 2H), 1.26 (s, 9H). MS (ESI) m/z: 401 (M+H).

To a solution of 2-(4-methoxy-benzyl)-1,1-dioxo-1λ⁶-[1,2,5]thiadiazolidin-3-one (64 mg, 0.25 mmol) in anhydrous DMF (2 mL) was added NaH (11 mg, 0.27 mmol) at 0° C. After stirred for 0.5 h at 0° C., a solution of 1-[5-t-butyl-2-(3-chloromethyl-phenyl)-2H-pyrazol-3-yl]-3-(4-fluoro-phenyl)-urea from the previous reaction (100 mg, 0.25 mmol) in anhydrous DMF (2 mL) was added to the reaction mixture, then was stirred overnight at RT. The crude was purified through prepared-HPLC to yield 1-(5-t-butyl-2-{3-[5-(4-methoxy-benzyl)-1,1,4-trioxo-1λ⁶-[1,2,5]thiadiazolidin-2-ylmethyl]-phenyl}-2H-pyrazol-3-yl)-3-(4-fluoro-phenyl)-urea (45 mg). ¹H NMR (DMSO-d₆): 8.95 (s, 1H), 8.37 (s, 1H), 7.50-7.54 (m, 3H), 7.36-7.41 (m, 3H), 7.25 (d, J=8.8 Hz, 2H), 7.07 (t, J=8.8 Hz, 2H), 6.87 (d, J=8.4 Hz, 2H), 6.35 (s, 1H), 4.64 (s, 2H), 4.47 (s, 2H), 4.19 (s, 2H), 3.75 (s, 3H), 1.26 (s, 9H). MS (ESI) m/z: 515 (M+H⁺).

A solution of 1-(5-t-butyl-2-{3-[5-(4-methoxy-benzyl)-1,1,4-trioxo-1λ⁶-[1,2,5]thiadia-zolidin-2-ylmethyl]-phenyl}-2H-pyrazol-3-yl)-3-(4-fluoro-phenyl)-urea (40 mg, 0.060 mmol) in trifluoroacetic acid (3 mL) was stirred at 50° C. for 4 h. After the solvent was removed, the residue was purified by preparative HPLC to afford 1-{5-t-butyl-2-[3-(3-(R)-methyl-1,1,4-trioxo-1λ⁶-1,2,5]thiadiazolidin-2-ylmethyl)-phenyl-2H-pyrazol-3-yl}-3-naphthalen-1-yl-urea as a white power (12 mg). ¹H NMR (DMSO-d₆): 8.98 (s, 1H), 8.39 (s, 1H), 7.37-7.51 (m, 6H), 7.07 (t, J=8.8 Hz, 2H), 6.35 (s, 1H), 4.21 (s, 2H), 3.88 (s, 2H), 1.26 (s, 9H). MS (ESI) m/z: 501 (M+H⁺).

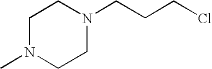

### Example GG

To a stirred suspension of K₂CO₃ (5.5 g, 40 mmol) and 1-bromo-3-chloro-propane (3.78 g, 24 mmol) in acetonitrile (10 mL) was added a solution of N-methyl piperazine (2.0 g, 20 mmol) in acetonitrile (10 mL) dropwise at RT. After the addition was completed, the reaction mixture was stirred for 3 h then filtered. The filtrate was concentrated and dissolved in CH₂Cl₂, washed with brine, dried (NaSO₄) and filtered. After removal of the solvent, the residue was dissolved in ether. To the above solution was added the solution of HCl and filtered to afford the desired product (2.3 g, 65.7%). ¹H NMR (D₂O): 3.61 (t, J=6.0 Hz, 2H), 3.59 (br, 8H), 3.31 (t, J=8.0 Hz, 2H), 2.92 (s, 3H), 2.15 (m, 2H).

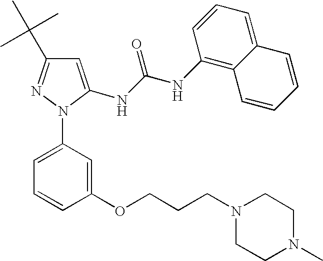

### Example 163

To a solution of Example 41 (100 mg, 0.25 mmol) in acetonitrile (10 mL) was added Example GG (75 mg, 0.30 mmol) and K₂CO₃ (172 mg, 1.25 mmol). The resulting mixture was stirred at 45° C. for 3 h before filtered. After the filtrate was concentrated, the residue was purified by preparative TLC to afford 1-(5-t-Butyl-2-{3-[3-(4-methyl-piperazin-1-yl)-propoxy]-phenyl}-2H-pyrazol-3-yl)-3-naphthalen-1-yl-urea (31 mg, 23%). ¹H-NMR (CD₃OD): 7.93 (m, 1H), 7.88 (m, 1H), 7.71 (d, J=8.4 Hz, 1H), 7.66 (d, J=7.6 Hz, 1H), 7.43-7.50 (m, 4H), 7.14 (m, 2H), 7.05 (m, 1H), 6.43 (s, 1H), 4.10 (t, J=6.0 Hz, 2H), 3.09-3.15 (br, 4H), 2.74-2.86 (br, 6H), 2.72 (s, 3H), 1.99 (t, J=6.8 Hz, 2H), 1.35 (s, 9H). MS (ESI) m/z: 541 (M+H⁺).

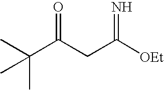

### Example HH

Example HH was synthesized according to literature procedures starting from 4,4-dimethyl-3-oxo-pentanenitrile (10 mmole) in absolute ethanol and HCl in quantitative afford.

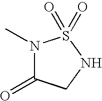

### Example II

To a stirred solution of chlorosulfonyl isocyanate (1.43 g, 10.0 mmol) in CH₂Cl₂ (20 mL) at 0° C. was added 2-methyl-propan-2-ol (0.74 g, 10.0 mmol) at such a rate that the reaction solution temperature did not rise above 5° C. After being stirred for 1.5 h, a solution of glycine ethyl ester (1.45 g, 12.0 mmol) and Et₃N (3.2 mL, 25.0 mmol) in CH₂Cl₂ (20 mL) was added at such a rate that the reaction temperature didn't rise above 5° C. When the addition was completed, the solution was warmed to RT and stirred overnight. The reaction mixture was poured into 10% HCl and extracted with CH₂Cl₂. The organic layer was washed with saturated NaCl, dried (Mg₂SO₄) and filtered. After-removal of the solvent, the crude product was washed with CH₂Cl₂ to afford ethyl 2-((N-(butyloxycarbonyl)sulfamoyl)amino)acetate (2.4 g, 85%). ¹H-NMR (DMSO): δ 10.85 (s, 1H), 8.04 (t, J=6.0 Hz, 1H), 4.07 (q, J=5.6 Hz, 2H), 3.77 (d, J=6.0 Hz, 2H), 1.40 (s, 9H), 1.18 (t, J=7.2 Hz, 3H).

To a solution of methanol (8.5 mmol) and triphenylphosphine (2.6 g, 8.5 mol) in dry THF is added a solution of ethyl 2-((N-(butyloxycarbonyl)sulfamoyl)amino)acetate from the previous step (2.4 g, 8.5 mol) and DIAD (2.0 g, 8.5 mmol) in dry THF dropwise at 0° C. under N₂ atmosphere. The mixture is stirred at 0° C. for 2 h, warmed to RT and is stirred overnight. After the solvent is removed in vacuo, the residue is purified by column chromatography to afford ethyl 2-((N-(butyloxycarbonyl)-N-methylsulfamoyl)amino)acetate.

To a solution of HCl in methanol (2 M) is added ethyl 2-((N-(butyloxycarbonyl)-N-methylsulfamoyl)amino)acetate from the previous step (5.0 mmol) in portions at RT and the mixture is stirred for 3 h. After the solvent is removed in vacuo, the residue is washed with diethyl ether to afford ethyl 2-((N-methylsulfamoyl)amino)acetate.

To a solution of ethyl 2-((N-methylsulfamoyl)amino)acetate from the previous step (3.5 mmol) in DMF (50 mL) is added KO-t-Bu (1.56 g, 13.88 mmol) in portions under N₂ at RT. The mixture is stirred overnight then quenched with HCl/methanol (2 M). After the solvent is removed in vacuo, the residue is washed with water to afford 2-methyl-1,1-dioxo-1λ⁶-[1,2,5]thiadiazolidin-3-one (480 mg, 54%). ¹H-NMR (CDCl₃): δ 7.36 (d, J=8.4 Hz, 2H), 6.87 (d, J=8.8 Hz, 2H), 4.87 (m, 1H), 4.68 (s, 2H), 4.03 (d, J=7.2 Hz, 2H), 3.80 (s, 3H).

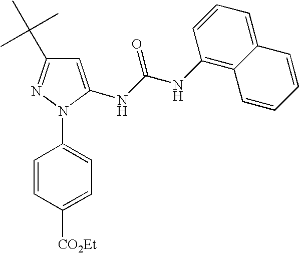

### Example 164

To a solution of Example X (2.9 g, 10 mmol) in THF (50 mL) was added a solution of 1-naphthyl isocyanate (1.7 g, 10 mmol) in THF (20 mL) at 0° C. The mixture was stirred at RT for 1 h and heated until all solids dissolved. The mixture was then stirred at RT for 3 h and poured into water (200 mL). The precipitate was filtered, washed with diluted HCl and H₂O, dried under vacuum to give 4.3 g of 4-[3-t-butyl-5-(3-naphthalen-1-yl-ureido)-pyrazol-1-yl]-benzoic acid ethyl ester, which was used without further purification.

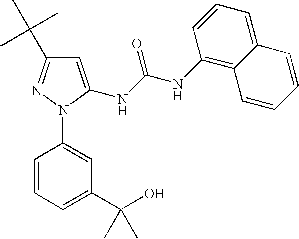

### Example 165

To a solution of Example B (228 mg, 0.5 mmol) in dry THF (20 mL) was added dropwise a solution of methyl magnesium bromide in toluene/THF (3.6 mL, 5.0 mmol) at −78° C. under N₂. After stirring for 1 h, the mixture was allowed to rise to RT and stirred for another 2 h. The reaction mixture was quenched with saturated NH₄Cl solution and aqueous HCl solution (10%), extracted with ethyl acetate. The combined organic extracts were washed with brine, dried (Na₂SO₄), the solvent removed in vacuo and the residue purified by column chromatography to afford 1-{5-t-butyl-2-[3-(1-hydroxy-1-methyl-ethyl)-phenyl]-2H-pyrazol-3-yl}-3-naphthalen-1-yl-urea (150 mg, 67%). ¹H NMR (DMSO-d6): 9.00 (s, 1H), 8.75 (s, 1H), 7.98 (d, J=7.6 Hz, 1H), 7.92-7.89 (m, 2H), 7.65-7.62 (m, 2H), 7.52-7.44 (m, 5H), 7.37 (d, J=6.8 Hz, 1H), 6.39 (s, 1H), 5.13 (s, 1H), 1.45 (s, 6H), 1.27 (s, 9H); MS (ESI) m/z: 443 (M+H⁺).

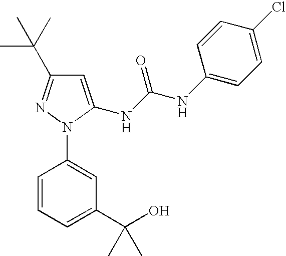

### Example 166

To a solution of Example C (220 mg, 0.5 mmol) in dry THF (20 mL) was added dropwise a solution of methyl magnesium bromide in toluene/H (3.6 mL, 5.0 mmol) at −78° C. under N₂. After stirring for 1 h, the mixture was allowed to rise to RT and stirred for another 2 h. The reaction mixture was quenched with saturated NH₄Cl and aqueous HCl solution (10%), and extracted with ethyl acetate. The combined organic extracts were washed with brine, dried (Na₂SO₄), the solvent was removed in vacuo and the residue was purified by column chromatography to afford 1-{5-t-butyl-2-[3-(1-hydroxy-1-methyl-ethyl)-phenyl]-2H-pyrazol-3-yl}-3-(4-chloro-phenyl)-urea (174 mg, 81%). ¹H NMR (DMSO-d₆): 9.11 (s, 1H), 8.34 (s, 1H), 7.59 (s, 1H), 7.46 (t, J=8.8 Hz, 1H), 7.43-7.40 (m, 3H), 7.31-7.28 (m, 3H), 6.34 (s, 1H), 5.13 (s, 1H), 1.42 (s, 6H), 1.27 (s, 9H); MS (ESI) m/z: 428 (M+H⁺).

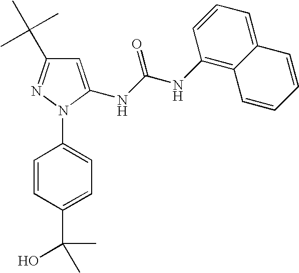

### Example 167

To a solution of Example 164 (228 mg, 0.5 mmol) in dry THF (20 mL) was added dropwise a solution of methylmagnesium bromide in toluene/THF (3.6 mL, 5.0 mmol) at −78° C. under N₂. After stirring for 1 h, the mixture was allowed to rise to RT and stirred for another 2 h. The reaction mixture was quenched with saturated NH₄Cl and aqueous HCl solution (10%), extracted with ethyl acetate. The combined organic extracts were washed with brine, dried (Na₂SO₄), the solvent was removed in vacuo and the residue purified by column chromatography to afford 1-{5-t-butyl-2-[4-(1-hydroxy-1-methyl-ethyl)-phenyl]-2H-pyrazol-3-yl}-3-naphthalen-1-yl-urea (180 mg, 81%). ¹H NMR (DMSO-d₆): 9.06 (s, 1H), 8.83 (s, 1H), 7.99 (d, J=8.0 Hz, 1H), 7.92 (t, J=8.0 Hz, 2H), 7.64-7.61 (m, 3H), 7.55-7.43 (m, 5H), 6.40 (s, 1H), 5.13 (s, 1H), 1.47 (s, 6H), 1.27 (s, 9H); MS (ESI) m/z: 443 (M+H⁺).

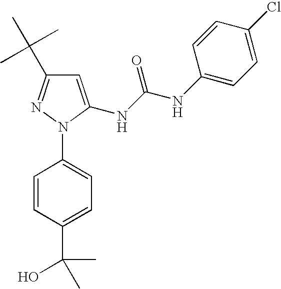

### Example 168

To a solution of Example 57 (220 mg, 0.5 mmol) in dry THF (20 mL) was added dropwise a solution of methyl magnesium bromide in toluene/THF (3.6 mL, 5.0 mmol) at −78° C. under N₂. After stirring for 1 h, the mixture was allowed to rise to RT and stirred for another 2 h. The reaction mixture was quenched with saturated NH₄Cl and aqueous HCl solution (10%), and extracted with ethyl acetate. The combined organic extracts were washed with brine, dried (Na₂SO₄), the solvent removed in vacuo and the residue was purified by column chromatography to afford 1-{5-t-butyl-2-[4-(1-hydroxy-1-methyl-ethyl)-phenyl]-2H-pyrazol-3-yl}-3-(4-chloro-phenyl)-urea (187 mg, 87%). ¹H-NMR (CDCl₃): 9.14 (s, 1H), 8.42 (s, 1H), 7.58 (d, J=8.4 Hz, 2H), 7.42 (d, J=5.6 Hz, 2H), 7.40 (d, J=4.8 Hz, 2H), 7.29 (d, J=8.8 Hz, 1H), 6.34 (s, 1H), 5.11 (s, 1H), 1.44 (s, 6H), 1.25 (s, 9H); MS (ESI) m/z: 427 (M+H⁺).

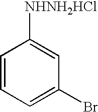

### Example JJ

To a solution of 3-bromo-phenylamine (17 g, 0.1 mol) in concentrated HCl (200 mL) was added an aqueous solution (20 mL) of NaNO₂ (7 g, 0.1 mol) at 0° C. and the resulting mixture was stirred for 1 h. A solution of SnCl₂.2H₂O (45 g, 0.2 mmol) in concentrated HCl (500 mL) was then added at 0° C. The reaction solution was stirred for an additional 2 h at RT. The precipitate was filtered and washed with ethanol and ether to give (3-bromo-phenyl)-hydrazine as a white solid, which was used for the next reaction without further purification.

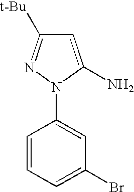

### Example KK

A mixture of Example JJ (22.2 g, 0.1 mol) and 4,4-dimethyl-3-oxo-pentanenitrile (18.7 g, 0.15 mol) in ethanol (250 mL) was heated to reflux overnight. The reaction solution was concentrated under reduced pressure, and the residue purified by column chromatography to afford 2-(3-bromo-phenyl)-5-t-butyl-2H-pyrazol-3-ylamine as a white solid. ¹H NMR (DMSO-d₆): 7.85 (s, 1H), 7.68 (d, J=7.6 Hz, 1H), 7.62 (d, J=7.2 Hz, 1H), 7.50 (t, J=8.0 Hz, 1H), 5.62 (s, 1H), 1.27 (s, 9H).

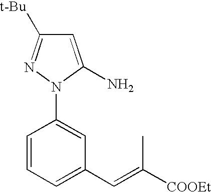

### Example LL

To a mixture of Example KK (2.94 g, 10 mmol), Pd(OAc)₂ (1 mmol), PPh₃ (20 mmol), and K₂CO₃ (20 mmol) in MeCN (50 mL) was added 2-methyl-acrylic acid ethyl ester (20 mmol). The resulting mixture was heated to reflux overnight, filtered, concentrated, and the residue was purified by column chromatography to afford 1.2 g of 3-[3-(5-Amino-3-t-butyl-pyrazol-1-yl)-phenyl]-2-methyl-acrylic acid ethyl ester. ¹H NMR (CDCl₃): 7.41 (s, 1H), 7.40-7.36 (m, 2H), 7.15 (d, J=6.8 Hz, 1H), 6.24 (s, 1H), 5.51 (s, 1H), 4.27 (q, J=7.2 Hz, 2H), 2.12 (s, 3H), 1.33 (s, 9H), 1.27 (t, J=7.2 Hz, 3H).

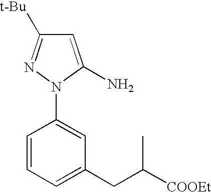

### Example MM

A mixture of Example LL (1.2 g,) and Pd/C (120 mg, 10%) in methanol (50 mL) was stirred under 40 psi of H₂ at RT overnight, filtered. And concentrated to afford 3-[3-(5-amino-3-t-butyl-pyrazol-1-yl)-phenyl]-2-methyl-propionic acid ethyl ester as a racemate (1.1 g), which was used for the next reaction without further purification.

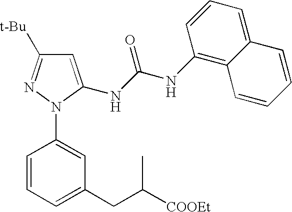

### Example 169

To a solution of Example MM (100 mg, 0.3 mmol) and Et₃N (60 mg, 0.6 mmol) in CH₂Cl₂ (10 mL) was added 1-isocyanato-naphthalene (77 mg, 0.45 mmol). The resulting mixture was stirred at RT overnight, added to water (50 mL), extracted with CH₂Cl₂ (3×30 mL) and the combined organic extracted were washed with brine, dried (Na₂SO₄), and filtered. After concentration under reduced pressure, the residue was purified by preparative-TLC to afford 3-(3-{3-t-butyl-5-[3-(4-fluoro-phenyl)-ureido]-pyrazol-1-yl}-phenyl)-propionic acid ethyl ester as a racemate (50 mg, 33%). ¹H-NMR (CDCl₃): 7.99 (s, 1H), 7.91 (d, J=8.4 Hz, 1H), 7.84 (t, J=7.2 Hz, 2H), 7.67 (d, J=8.4 Hz, 1H), 7.49-7.41 (m, 3H), 7.35-7.33 (m, 3H), 7.21 (s, 1H), 7.14-7.13 (m, 1H), 6.65 (s, 1H), 3.98 (q, J=6.0 Hz, 2H), 2.92-2.88 (m, 3H), 1.36 (s, 9H), 1.24 (d, J=6.0 Hz, 3H), 1.08 (t, J=7.2 Hz, 3H); MS (ESI) m/z: 499 (M+H⁺).

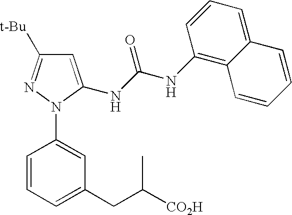

### Example 170

A solution of Example 169 (17 mg, mmol) and 2N LiOH (3 mL) in MeOH (3 mL) was stirred at RT over night. The reaction mixture was adjusted to pH=4, and extracted with ethyl acetate (3×20 mL). The combined organic extracts were washed with brine, dried (Na₂SO₄), and filtered. After the filtrate was concentrated, the residue was purified by preparative-TLC to afford 3-{3-[3-t-butyl-5-(3-naphthalen-1-yl-ureido)-pyrazol-1-yl]-phenyl}-2-methyl-propionic acid as a racemate (15 mg, 92%). ¹H NMR (DMSO): 11.81 (br s, 1H), 9.58 (s, 1H), 8.56 (s, 1H), 7.95 (d, J=7.6 Hz, 1H), 7.82 (d, J=8.4 Hz, 1H), 7.55 (d, J=7.6 Hz, 1H), 7.45-7.35 (m, 5H), 7.28 (d, J=8.0 Hz, 1H), 7.14 (t, J=7.6 Hz, 1H), 6.52 (s, 1H), 3.77 (m, 1H), 2.65 (m, 1H), 2.36 (m, 1H), 1.27 (s, 9H), 1.00 (d, J=6.8 Hz, 3H); MS (ESI) m/z: 471 (M+H⁺).

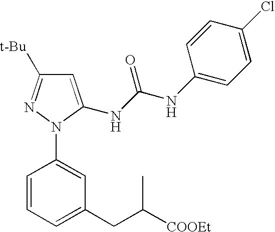

### Example 171

To a solution of Example MM (100 mg, 0.3 mmol) and Et₃N (60 mg, 0.6 mmol) in CH₂Cl₂ (10 mL) was added 1-chloro-4-isocyanato-benzene (77 mg, 0.45 mmol). The resulting mixture was stirred at RT overnight, and then added to water (50 mL). The solution was extracted with CH₂Cl₂ (3×30 nm) and the combined organic extracts were washed with brine, dried (Na₂SO₄), and filtered. After concentration under reduced pressure, the residue was purified by preparative-TLC to afford 3-(3-{3-t-butyl-5-[3-(4-chloro-phenyl)-ureido]-pyrazol-1-yl}-phenyl)-2-methyl-propionic acid ethyl ester as a racemate (51 mg, 35%). ¹H-NMR (CDCl₃): 8.20 (s, 1H), 7.39 (d, J=4.4 Hz, 2H), 7.37 (d, J=8.8 Hz, 2H), 7.21 (t, J=8.4 Hz, 2H), 7.14-7.11 (m, 2H), 6.59 (s, 1H), 4.04-3.99 (m, 2H), 3.00 (m, 1H), 2.93 (m, 1H), 2.83 (m, 1H), 1.34 (s, 9H), 1.17 (d, J=6.4 Hz, 3H), 1.15 (t, J=7.2 Hz, 3H); MS (ESI) m/z: 483 (M+H⁺).

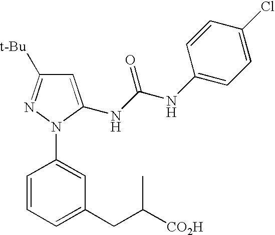

### Example 172

A solution of Example 171 (15 mg, mmol) and 2N LiOH (3 mL) in MeOH (3 mL) was stirred at RT overnight. The reaction mixture was adjusted to pH=4, extracted with ethyl acetate (3×20 mL), the combined organic extracts were washed with brine, dried (Na₂SO₄), and filtered. After the filtrate was concentrated, the residue was purified by preparative-TLC to afford 3-(3-{3-t-butyl-5-[3-(4-chloro-phenyl)-ureido]-pyrazol-1-yl}-phenyl)-2-methyl-propionic acid as a racemate (13 mg, 90%). ¹H NMR (DMSO): 12.48 (br s, 1H), 9.35 (br s, 1H), 7.55 (d, J=8.8 Hz, 1H), 7.34-7.32 (m, 2H), 7.26 (d, J=8.4 Hz, 1H), 7.24 (d, J=8.8 Hz, 2H), 7.10 (d, J=7.6 Hz, 1H), 6.45 (s, 1H), 2.74 (m, 1H), 2.65 (m, 1H), 2.31 (m, 2H), 1.26 (s, 9H), 0.99 (d, J=6.8 Hz, 3H); MS (ESI) m/z: 455 (M+H⁺).

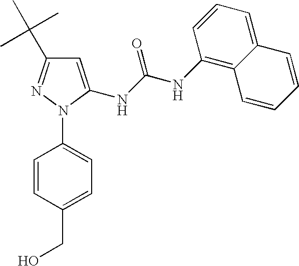

### Example 173

To a stirred solution of Example 164 (500 mg, 0.83 mmol) in THF (10 mL) was added LiAlH₄ powder (65 mg, 1.66 mmol) in portion at 0° C. under N₂. The mixture was stirred for 2 h at RT, excess LiAlH₄ was destroyed by a slow addition of ice, and the reaction mixture was acidified to pH=7 with dilute HCl. After the solvent was removed, the residue was extracted with ethyl acetate. The combined organic extracts were washed with brine, dried (Na₂SO₄), and filtered. After concentration in vacuo, the crude product was purified by preparative-TLC to afford 1-[2-(4-hydroxymethyl-phenyl)-5-isopropyl-2H-pyrazol-3-yl]-3-naphthalen-1-yl-urea (415 mg, 92%). ¹H NMR (DMSO-d₆): 9.04 (s, 1H), 8.78 (s, 1H), 7.98 (d, J=8.0 Hz, 1H), 7.90 (d, J=7.2 Hz, 2H), 7.63 (d, J=8.4 Hz, 1H), 7.55-7.42 (m, 7H), 6.39 (s, 1H), 5.30 (t, J=5.6 Hz, 1H), 4.56 (d, J=5.6 Hz, 2H), 1.27 (s, 9H); MS (ESI) m/z: 415 (M+H⁺).

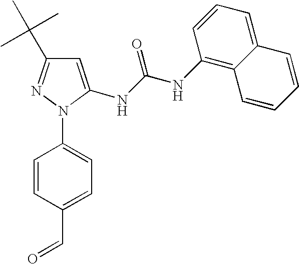

### Example 174

To a solution of Example 173 (200 mg) in CH₂Cl₂ (50 mL) was added MnO₂ (450 mg) at RT. The suspension was stirred for 2 h then filtered through celite. The filtrate was concentrated under reduced pressure to afford 150 mg of 1-[5-t-butyl-2-(4-formyl-phenyl)-2H-pyrazol-3-yl]-3-naphthalen-1-yl-urea, which was used without further purification.

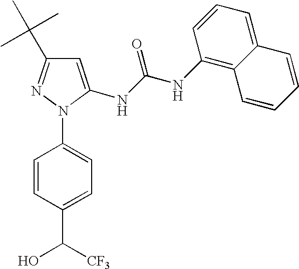

### Example 175

To a solution of (trifluoromethyl)trimethylsilane (77 mg) and TBAF (10 mg) in THF (10 mL) was added Example 174 (150 mg) in THF (10 mL) under N₂ atmosphere in ice-bath. The resulting mixture was stirred at 0° C. for 1 h and then warmed to RT for an additional hour. To the reaction was then added 0.5 mL of 3 N HCL, which was then stirred at RT overnight. After removal the solvent, the residue was dissolved in CH₂Cl₂ (50 mL). The organic layer was washed with saturated NaHCO₃ and brine, dried (Na₂SO₄), and filtered. After the filtrate was concentrated under reduced pressure, the residue was purified by preparative-TLC to afford the final product 1-{5-t-Butyl-2-[4-(2,2,2-trifluoro-1-hydroxy-ethyl)-phenyl]-2H-pyrazol-3-yl}-3-naphthalen-1-yl-urea (110 mg, 63%). ¹H NMR (DMSO-d₆)-9.07 (s, 1H), 8.89 (s, 1H), 8.03 (d, J=8.0 Hz, 1H), 7.90 (d, J=7.6 Hz, 2H), 7.67-7.62 (m, 5H), 7.55-7.51 (m, 2H), 7.44 (t, J=8.0 Hz, 1H), 6.95 (d, J=6.0 Hz, 1H), 6.42 (s, 1H), 5.27 (m, 1H), 1.28 (s, 9H). MS (ESI) m/z: 483 (M+H⁺).

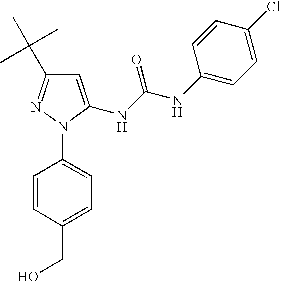

### Example 176

To a stirred solution of Example 57 (500 mg, 1.1 mmol) in THF (10 mL) was added LiAlH₄ powder (65 mg, 1.66 mmol) in portion at 0° C. under N₂. The mixture was stirred for 2 h at RT, excess LiAlH₄ was destroyed by a slow addition of ice, and the reaction mixture was acidified to pH=7 with diluted HCl. After the solvent removal, the residue was extracted with ethyl acetate, and the combined organic extracts were washed with brine, d dried (Na₂SO₄), and filtered, After solvent removal, the crude product was purified by preparative TLC to 1-[5-t-butyl-2-(4-hydroxymethyl-phenyl)-2H-pyrazol-3-yl]-3-(4-chloro-phenyl)-urea (380 mg, 92%) as a white powder. ¹H-NMR (CDCl₃): 8.17 (br s, 1H), 7.22 (s, 4H), 7.17 (d, J=8.0 Hz, 2H), 7.09 (d, J=8.0 Hz, 2H), 7.04 (s, 1H), 6.38 (s, 1H), 4.51 (s, 1H), 1.22 (s, 9H); MS (ESI) m/z: 399 (M+H⁺).

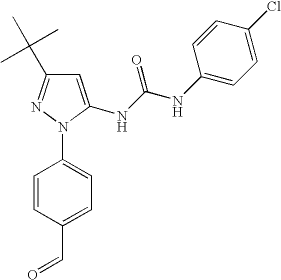

### Example 177

To a solution of Example 176 (200 mg) in CH₂Cl₂ (50 mL) was added MnO₂ (450 mg) at RT. The suspension was stirred for 2 h, then filtered through celite. The filtrate was concentrated to afford 160 mg of 1-[5-t-butyl-2-(4-formyl-phenyl)-2H-pyrazol-3-yl]-3-(4-chloro-phenyl)-urea, which was used without further purification.

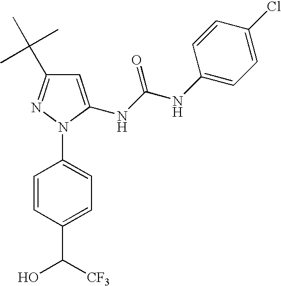

### Example 178

To a solution of (trifluoromethyl)trimethylsilane (86 mg) and TBAF (10 mg) in THF (10 mL) was added Example 177 (160 mg) in THF (20 mL) under N₂ atmosphere in ice-bath. The resulting mixture was stirred at 0° C. for 1 h and then warmed to RT for an additional hour. To the reaction was added 0.5 mL of 3 N HCl, which was then stirred at RT overnight. After removal of the solvent, the residue was dissolved in CH₂Cl₂ (100 mL). The organic extracts were washed with saturated NaHCO₃ and brine, dried (Na₂SO₄), and filtered. After the filtrate was concentrated under reduced pressure, the residue was purified by preparative-TLC to afford the final product 1-{5-t-butyl-2-[4-(2,2,2-trifluoro-1-hydroxy-ethyl)-phenyl]-2H-pyrazol-3-yl}-3-(4-chloro-phenyl)-urea (120 mg, 64%). ¹H-NMR (DMSO-d₆): 9.15 (s, 1H), 8.50 (s, 1H), 7.61 (d, J=8.4 Hz, 2H), 7.55 (d, J=8.4 Hz, 2H), 7.42 (d, J=8.8 Hz, 2H), 7.28 (d, J=8.8 Hz, 2H), 6.91 (d, J=5.6 Hz, 1H), 6.36 (s, 1H), 5.25 (m, 1H), 1.26 (s, 9H); MS (ESI) m/z: 467 (M+H⁺).

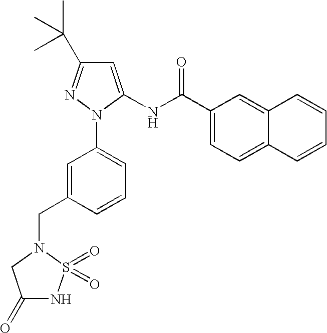

### Example 179

Example CC, 2-naphthoic acid chloride and Example DD were combined utilizing the same general approach for Example 162 to yield N-(3-tert-butyl-1-(3-([5-1,1,4-trioxo-1λ⁶-[1,2,5]thiadiazolidin-2-ylmethyl]phenyl)-1H-pyrazol-5-yl)-2-naphthamide. ¹H-NMR (DMSO-d₆): 10.50 (s, 1H), 8.45 (s, 1H), 8.15-8.05 (m, 3H), 7.90 (s, 1H), 7.60 (t, J=7.2 Hz, 3H), 7.45 (s, 1H), 7.38 (t, J=8.0 Hz, 1H), 7.27 (d, J=7.2 Hz, 1H), 6.44 (s, 1H), 4.05 (s, 2H), 1.31 (s, 9H). MS (ESI) m/z: 518 (M+H⁺).

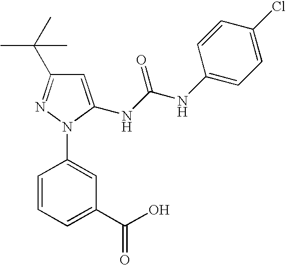

### Example 180

Example C was reacted with LiOH utilizing the procedure for Example 146 to yield 3-(3-t-butyl-5-(3-(4-chlorophenyl)ureido)-1H-pyrazol-1-yl)benzoic acid in 90% overall yield. ¹H NMR (DMSO-d₆): 9.00 (s, 1H), 8.83 (s, 1H), 8.25-7.42 (m, 1H), 6.42 (s, 1H), 1.26 (s, 9H); MS (ESI): Expected: 412.88 Found: 413.00.

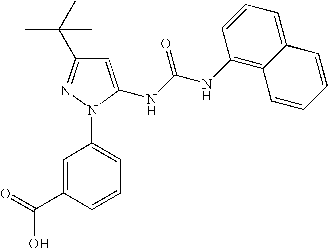

### Example 181

Example B was reacted with LiOH utilizing for procedure for Example 146 to yield 3-(3-t-butyl-5-(3-(naphthalen-1-yl)ureido)-1H-pyrazol-1-yl)benzoic acid in 90% overall yield. ¹H NMR (DMSO-d₆): δ 9.11 (s, 1H), 8.47 (s, 1H), 8.06 (m, 1H), 7.93 (d, J=7.6 Hz, 1H), 7.81 (d, J=8.0 Hz, 1H), 7.65 (dd, J=8.0, 7.6 Hz, 1H), 7.43 (d, J=8.8 Hz, 2H), 7.30 (d, J=8.8 Hz, 2H), 6.34 (s, 1H), 1.27 (s, 9H); MS (ESI) Expected: 428.49 Found: 429.2 (M+1).

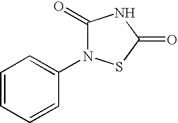

### Example NN

To the solution of phenyl-urea (13.0 g, 95.48 mol) in THF (100 mL) was slowly added chlorocarbonyl sulfenylchloride (13 mL, 148.85 mmol) at RT. The reaction mixture was refluxed overnight, the volatiles removed in vacuo yielded 2-phenyl-1,2,4-thiadiazolidine-3,5-dione as a white solid (4.0 g, 20%). ¹H NMR (DMSO-d₆): δ 12.49 (s, 1H), 7.51 (d, J=8.0 Hz, 2H), 7.43 (t, J=7.6 Hz, 2H), 7.27 (t, J=7.2 Hz, 1H).

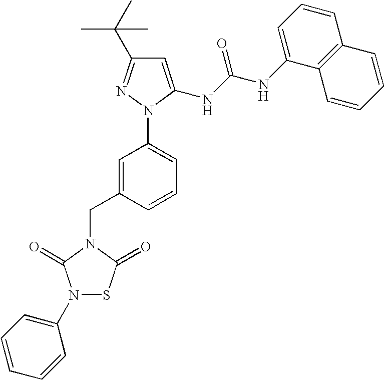

### Example 182

Example E and Example NN were reacted together utilizing the same general approach as for Example 160 to afford 1-(3-t-butyl-1-(3-((3,5-dioxo-2-phenyl-1,2,4-thiadiazolidin-4-yl)methyl)phenyl)-1H-pyrazol-5-yl)-3-(naphthalen-1-yl)urea. ¹H NMR (DMSO-d₆): δ8.96 (s, 1H), 8.01-7.21 (m, 16H), 6.40 (s, 1H), 4.85 (s, 2H), 1.28 (s, 9H); MS (ESI): Expected: 590.21, Found: 591.26 (M+1).

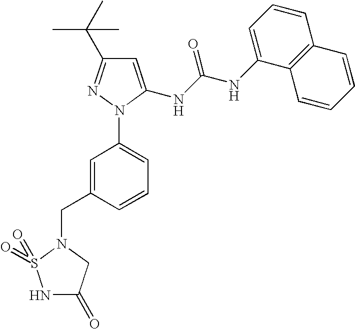

### Example 183

Example CC, 1-naphthylisocyanate and Example DD were combined utilizing the same general approach for Example 162 to yield 1-(5-t-butyl-2-{3-[5-1,1,4-trioxo-1λ⁶-[1,2,5]thiadiazolidin-2-ylmethyl]-phenyl}-2H-pyrazol-3-yl)-1-naphthylurea. ¹H NMR (DMSO-d₆): δ 9.0 (s, 1H), 8.81 (s, 1H), 7.99-7.42 (m, 11H), 6.41 (s, 1H), 4.33 (s, 2H), 1.27 (s, 9H); MS (ESI) Exact Mass: 532.19 Found: =533.24

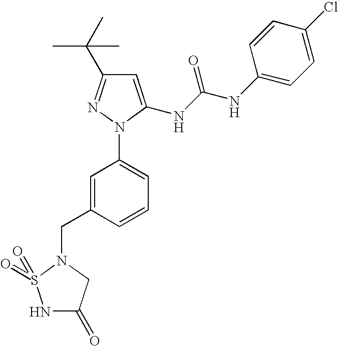

### Example 184

Example CC, p-chlorophenylisocyanate and Example DD were combined utilizing the same general approach for Example 162 to yield 1-(5-t-butyl-2-{3-[5-1,1,4-trioxo-1λ⁶-[1,2,5]thiadiazolidin-2-ylmethyl]-phenyl}-2H-pyrazol-3-yl)-3-(4-chloro-phenyl)-urea. ¹H NMR (DMSO-d₆): δ 9.07 (s, 1H), 8.42 (s, 1H), 7.52-7.272 (m, 8H), 6.36 (s, 1H), 4.60 (s, 2H), 1.26 (s, 9H); MS (ESI) Exact Mass: 516.13 Found: =517.1

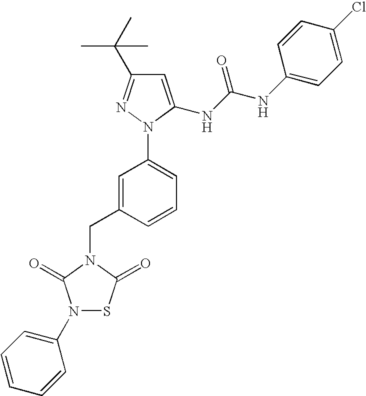

### Example 185

Example G and Example NN were reacted together utilizing the same general approach as for Example 160 to afford 1-(3-t-butyl-1-(3-((3,5-dioxo-2-phenyl-1,2,4-thiadiazolidin-4-yl)methyl)phenyl)-1H-pyrazol-5-yl)-3-(4-chlorophenyl)urea. ¹H NMR (DMSO-d₆): δ9.02 (s, 1H), 8.51 (s, 1H), 7.52-7.24 (m, 13H), 6.36 (s, 1H), 4.90 (s, 2H), 1.27 (s, 9H); MS (ESI): Expected: 574.16 Found: 575.26 (M+1)

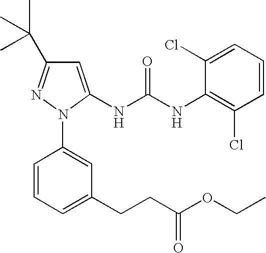

### Example 186

Example Z and 2,6-dichlorophenylisocyanate were reacted utilizing the same conditions as for Example 145 to yield ethyl 3-(3-(3-t-butyl-5-(3-(2,6-dichlorophenyl)ureido)-1H-pyrazol-1-yl)phenyl)propanoate. ¹H NMR (DMSO-d₆): δ 7.46-7.26 (m, 7H), 6.35 (s, 1H), 4.11 (q, J=7.2 Hz, 2H), 3.31 (t, J=5.2 Hz, 2H), 2.68 (t, J=5.6 Hz, 2H), 1.32 (s, 9H), 1.24 (t, J=7.2 Hz, 3H); MS (ESI): Expected: 502.15 Found: =503.1 (M+1).

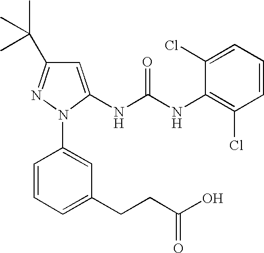

### Example 187

Example 186 was reacted utilizing the same condition as for Example 146 to yield 3-(3-(3-t-butyl-5-(3-(2,6-dichlorophenyl)ureido)-1H-pyrazol-1-yl)phenyl)propanoic acid in >90% yield. ¹H NMR (DMSO-d₆): δ 8.70 (s, 1H), 8.60 (s, 1H) 7.50-7.24 (m, 7H), 6.26 (s, 1H), 2.87 (t, J=5.2 Hz, 2H), 2.57 (t, J=5.6 Hz, 2H), 1.25 (s, 9H); MS (ESI): Expected: 474.12 Found: 475.18 (M+1).

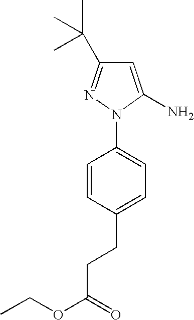

### Example OO

A mixture of ethyl 3-(4-aminophenyl) acylate (1.5 g) and 10% Pd on activated carbon (0.3 g) in ethanol (20 ml) was hydrogenated at 30 psi for 18 h and filtered over Celite. Removal of the volatiles in vacuo provided ethyl 3-(4-aminophenyl)propionate (1.5 g).

A solution of the crude material from the previous reaction (1.5 g, 8.4 mmol) was dissolved in 6 N HCl (9 ml), cooled to 0° C., and vigorously stirred. Sodium nitrite (0.58 g) in water (7 ml) was added. After 1 h, tin (II) chloride dihydrate (5 g) in 6 N HCl (10 ml) was added. The reaction mixture was stirred at 0° C. for 3 h. The pH was adjusted to pH 7 to yield ethyl 3-(4-(3-t-butyl-5-amino-1H-pyrazol-1-yl)phenyl)propanoate.

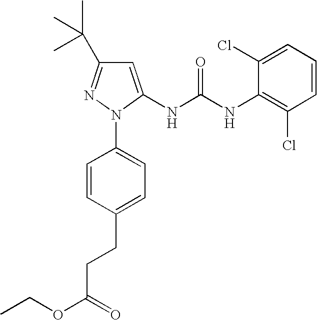

### Example 188

Example OO and 2,6-dichlorophenylisocyanate were reacted utilizing the same conditions as for Example 145 to yield ethyl 3-(4-(3-t-butyl-5-(3-(2,6-dichlorophenyl)ureido)-1H-pyrazol-1-yl)phenyl)propanoate. ¹H NMR (DMSO-d₆): δ 7.45-7.24 (m, 7H), 6.36 (s, 1H), 4.10 (q, J=7.2 Hz, 2H), 3.02 (t, J=5.2 Hz, 2H), 2.70 (t, J=5.6 Hz, 2H), 1.33 (s, 9H), 1.22 (t, J=7.2 Hz, 3H); MS (ESI): Expected: 502.15 Found: =503.1 (M+1).

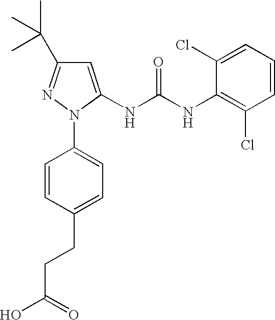

### Example 189

Example 188 was reacted utilizing the same condition as for Example 146 to yield 3-(3-(3-t-butyl-5-(3-(2,6-dichlorophenyl)ureido)-1H-pyrazol-1-yl)phenyl)propanoic acid in >90% yield. ¹H NMR (DMSO-d₆): δ 8.66 (s, 1H), 8.58 (s, 1H) 7.50-7.28 (m, 7H), 6.27 (s, 1H), 2.85 (t, J=5.2 Hz, 2H), 2.48 (t, J=5.6 Hz, 2H), 1.24 (s, 9H); MS (ESI): Expected: 474.12 Found: 475.18 (M+1).

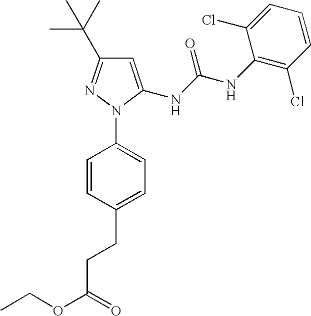

### Example 190

Example OO and p-chlorophenylisocyanate were reacted utilizing the same conditions as for Example 145 to yield ethyl 3-(4-(3-tert-butyl-5-(3-(4-chlorophenyl)ureido)-1H-pyrazol-1-yl)phenyl)propanoate. ¹H NMR (DMSO-d₆): δ 7.34-7.19 (m, 9H), 6.36 (s, 1H), 4.10 (q, J=7.2 Hz, 2H), 2.92 (t, J=5.2 Hz, 2H), 2.58 (t, J=5.6 Hz, 2H), 1.32 (s, 9H), 1.25 (t, J=7.2 Hz, 3H); MS (ESI): Exact Mass: 468.19 Found: =469.21 (M+1).

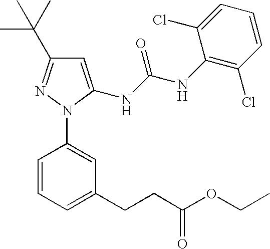

### Example 191

Example Z and p-chlorophenylisocyante were reacted utilizing the same conditions as for Example 145 to yield ethyl 3-(3-(3-tert-butyl-5-(3-(4-chlorophenyl)ureido)-1H-pyrazol-1-yl)phenyl)propanoate. ¹H NMR (DMSO-d₆): δ 9.12 (s, 1H), 8.37 (s, 1H), 7.41-7.27 (m, 8H), 6.34 (s, 1H), 5.73 (s, 1H), 4.01 (q, J=7.2 Hz, 2H), 2.90 (t, J=5.2 Hz, 2H), 2.62 (t, J=5.6 Hz, 2H), 1.25 (s, 9H), 1.125 (t, J=7.2 Hz, 3H); MS (ESI): Exact Mass: 468.19 Found: =469.21 (M+1).

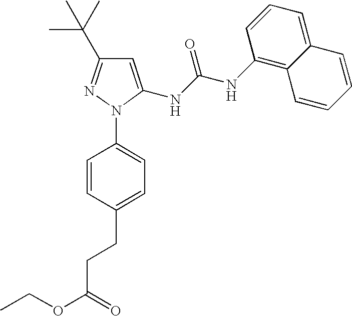

### Example 192

Example OO and 1-naphthylisocyanate were reacted utilizing the same conditions as for Example 145 to yield ethyl 3-(4-(3-tert-butyl-5-(3-(naphthalen-1-yl)ureido)-1H-pyrazol-1-yl)phenyl)propanoate. δ 7.88-9.95 (m, 13H), 6.27 (s, 1H), 4.04 (q, J=7.2 Hz, 2H), 2.75 (t, J=5.2 Hz, 2H), 2.42 (t, J=5.6 Hz, 2H), 1.27 (s, 9H), 1.20 (t, J=7.2 Hz, 3H); MS (ESI): Exact Mass: 484.25 Found: =485.26 (M+1).

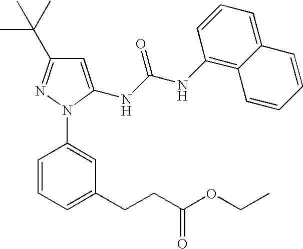

### Example 193

Example Z and 1-naphthylisocyanate were reacted utilizing the same conditions as for Example 145 to yield ethyl 3-(3-(3-tert-butyl-5-(3-(naphthalen-1-yl)ureido)-1H-pyrazol-1-yl)phenyl)propanoate. ¹H NMR (DMSO-d₆): δ 9.01 (s, 1H), 8.80 (s, 1H), 8.0-7.27 (m, 11H), 6.41 (s, 1H), 4.01 (q, J=7.2 Hz, 2H), 2.95 (t, J=5.2 Hz, 2H), 2.72 (t, J=5.6 Hz, 2H), 1.27 (s, 9H), 1.15 (t, J=7.2 Hz, 3H); MS (ESI): Exact Mass: 484.25 Found: =485.26 (M+1).

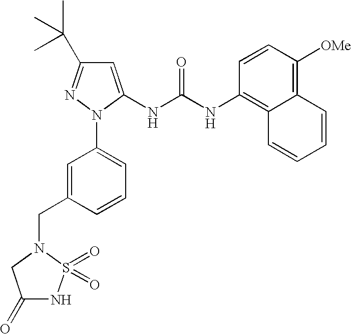

### Example 194

Example CC, 1-(4-methoxynaphthyl)isocyanate and Example DD were combined utilizing the same general approach for Example 162 to yield 1-(5-t-butyl-2-{3-[5-1,1,4-trioxo-1λ⁶-[1,2,5]thiadiazolidin-2-ylmethyl]-phenyl}-2H-pyrazol-3-yl)-1-(4-methoxynaphthyl)urea. ¹H NMR (DMSO-d₆): δ 8.69 (s, 1H), 8.61 (s, 1H), 8.15-6.90 (m, 10H), 6.36 (s, 1H), 4.37 (s, 2H), 3.93 (s, 3H), 1.22 (s, 9H); MS (ESI) Exact Mass: 562.20 Found: 563.2.

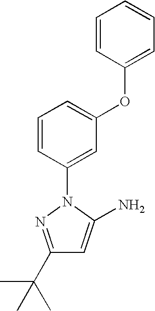

### Example PP

In a 250 mL Erlenmyer flask with a magnetic stir bar, 3-phenoxyphenylamine (4.81 g, 0.026 mol) was added to 6 N HCl (40 mL) and cooled with an ice bath to 0° C. A solution of NaNO₂ (2.11 g, 0.0306 mol, 1.18 eq.) in water (5 mL) was added drop wise. After 30 min, SnCl₂.2H₂O (52.0 g, 0.23 mol, 8.86 eq.) in 6 N HCl (100 mL) was added and the reaction mixture was allowed to stir for 3 h, and then subsequently transferred to a 500 mL round bottom flask. To this, 4,4-dimethyl-3-oxopentanenitrile (3.25 g, 0.026 mol) and EtOH (100 ml) were added and the mixture refluxed for 4 h, concentrated in vacuo and the residue extracted with EtOAc (2×100 mL) and purified by column chromatography using hexane/EtOAc/Et₃N (8:2:0.2) to yield 3-tert-butyl-1-(3-phenoxyphenyl)-1H-pyrazol-5-amine (1.40 g, 17%). mp: 108-110° C.; ¹H NMR (CDCl₃): δ 7.3 (m, 10H), 5.7 (s, 1H), 4.9 (brs, 2H), 1.3 (s, 9H).

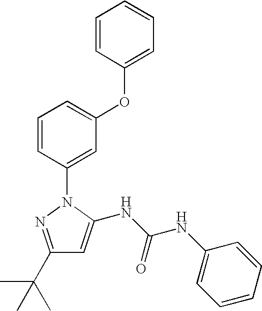

### Example 195

In a dry vial with a magnetic stir bar, Example PP (0.184 g; 0.60 mmol) was dissolved in 2 mL CH₂Cl₂ (anhydrous) followed by the addition of phenylisocyanate (0.0653 mL; 0.60 mmol; 1 eq.). The reaction was kept under Ar and stirred for 18 h. Evaporation of solvent gave a crystalline mass that was recrystallized from EtOAc/hexane and then filtered washing with hexane/EtOAc (4:1) to yield 1-[3-tert-butyl-1-(3-phenoxyphenyl)-1H-pyrazol-5-yl]-3-phenylurea (0.150 g, 50%). HPLC purity: 96%; ¹H NMR (CDCl₃): δ 7.5 (m, 16H), 6.8 (s, 1H), 6.5 (s, 1H), 1.4 (s, 9H).

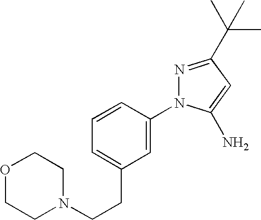

### Example QQ

To a stirred solution of Example L (1.2 g, 3.5 mmol) in THF (6 ml) was added borane-methylsulfide (9 mmol). The mixture was heated to reflux for 90 min and cooled to RT, and 6 N HCl was added and heated to reflux for 10 min. The mixture was basified by adding sodium hydroxide, followed by extraction with ethyl acetate. The organic layer was dried (Na₂SO₄) filtered and concentrated in vacuo to yield 3-tert-butyl-1-[3-(2-morpholinoethyl)phenyl]-1H-pyrazol-5-amine (0.78 g), which was used without further purification.

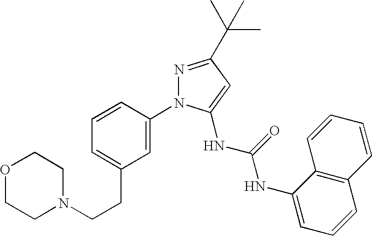

### Example 196

A mixture of Example QQ (0.35 g, 1.07 mmol) and 1-naphthylisocyanate (0.18 g, 1.05 mmol) in dry CH₂Cl₂ (4 ml) was stirred at RT under N₂ for 18 h. The solvent was removed in vacuo and the crude product was purified by column chromatography using 5% methanol in CH₂Cl₂ (with a small amount of TEA) as the eluent (0.18 g, off-white solid) to yield 1-{3-tert-butyl-1-[3-(2-morpholinoethyl)phenyl]-1H-pyrazol-5-yl}-3-naphthalen-1-yl)urea. mp: 88-90° C.; ¹H NMR (200 MHz, DMSO-d₆): δ 9.07 (s, 1H), 8.80 (s, 1H), 8.06-7.92 (m, 3H), 7.69-7.44 (m, 7H), 7.40-7.29 (m, 1H), 6.44 (s, 1H), 3.57-3.55 (m, 4H), 3.33-3.11 (m, 4H), 2.40-2.38 (m, 4H), 1.32 (s, 9H); MS

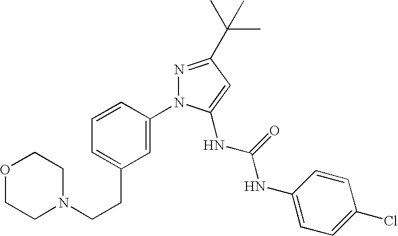

### Example 197

The title compound was synthesized in a manner analogous to Example 23 utilizing Example QQ (0.35 g, 1.07 mmol) and 4-chlorophenylisocyanate (0.165 g, 1.05 mmol) to yield 1-{3-tert-butyl-1-[3-(2-morpholinoethyl)phenyl]-1H-pyrazol-5-yl}-3-(4-chlorophenyl)urea. mp: 82-84° C.; ¹H NMR (200 MHz, DMSO-d₆): δ 9.18 (s, 1H, s), 8.40 (s, 1H), 7.53-7.26 (m, 8H), 6.37 (s, 1H), 3.62-3.54 (m, 4H), 2.82-2.78 (m, 4H), 2.41-2.39 (m, 4H), 1.30 (s, 9H); MS

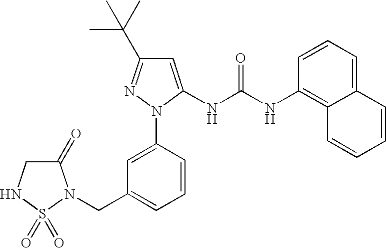

### Example 198

A mixture of compound 1,1-Dioxo-[1,2,5-]thiadiazolidin-3-one (94 mg, 0.69 mmol) and NaH (5.5 mg, 0.23 mmol) in THF (2 mL) was stirred at −10° C. under N₂ for 1 h until all NaH was dissolved. Example E (100 mg, 0.23 mmol) was added and the reaction was allowed to stir at RT overnight, quenched with H₂O, and extracted with CH₂Cl₂. The combined organic layers were concentrated in vacuo and the residue was purified by preparative HPLC to yield 1-(3-tert-butyl-1-{[3-(1,1,3-trioxo-[1,2,5]thiadiazolidin-2-yl)methyl]phenyl}-1H-pyrazol-5-yl)-3-(naphthalen-1-yl)urea (18 mg) as a white powder. ¹H NMR (CD₃OD): δ 7.71-7.44 (m, 1H), 6.45 (s, 1H), 4.83 (s, 2H), 4.00 (s, 2H), 1.30 (s, 9H). MS (ESI) m/z: 533.40 (M+H⁺).

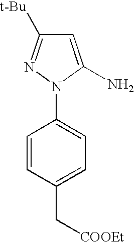

### Example RR

To a suspension of (4-amino-phenyl)acetic acid (20 g, 0.13 mol) in 150 mL of conc. HCl was added dropwise a solution of NaNO₂ (13.8 g, 0.2 mol) in H₂O at 0° C. The mixture was stirred for 1 h, after which a solution of SnCl₂.2H₂O (67 g, 0.3 mol) in conc. HCl was added dropwise at such a rate that the reaction mixture never rose above 5° C. The resulted mixture was stirred for 2 h. The precipitate was collected by suction and washed with Et₂O to afford 17 g of (4-hydrazino-phenyl)acetic acid hydrochloride. MS (ESI) m/z: 167 (M+H⁺).

A solution of (4-hydrazino-phenyl)acetic acid hydrochloride (17 g, 84 mmol) and 4,4-dimethyl-3-oxo-pentanenitrile (12.5 g, 0.1 mol) in EtOH (100 mL) containing conc. HCl (25 mL) was heated at reflux overnight. After removal of the solvent, the residue was washed with Et₂O to afford 22 g of [4-(5-amino-3-t-butyl-pyrazol-1-yl)-phenyl]acetic acid hydrochloride. MS (ESI) m/z: 274 (M+H⁺).

To a solution of [4-(5-amino-3-t-butyl-pyrazol-1-yl)-phenyl]acetic acid hydrochloride (22 g, 71 mmol) in EtOH (250 mL) cooled in an ice-water bath was added dropwise SOCl₂ (40 mL). The mixture was heated to reflux for 2 h. After removal of the solvent, the residue was washed with Et₂O to afford 22.5 g of ethyl 2-(4-(3-t-butyl-5-amino-1H-pyrazol-1-yl)phenyl)acetate. ¹H NMR (300 MHz, DMSO-d₆), 87.55-7.45 (m, 4H), 5.61 (s, 1H), 4.08 (q, J=6.9 Hz, 2H), 3.77 (s, 2H), 1.27 (s, 9H), 1.19 (t, J=6.9 Hz, 3H); MS (ESI) m/z: 302 (M+H⁺)

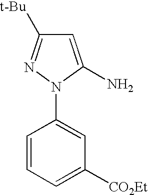

### Example SS

To a solution of 3-aminobenzoic acid (200 g, 1.46 mol) in conc. HCl (200 mL) was added an aqueous solution (250 mL) of NaNO₂ (102 g, 1.46 mol) at 0° C. The reaction mixture was stirred for 1 h and a solution of SnCl₂.2H₂O (662 g, 2.92 mol) in conc. HCl (2 L) was then added at 0° C., and the reaction stirred for an additional 2 h at RT. The precipitate was filtered and washed with EtOH and Et₂O to yield 3-hydrazinobenzoic acid hydrochloride as a white solid.

The crude material from the previous reaction (200 g, 1.06 mol) and 4,4-dimethyl-3-oxo-pentanenitrile (146 g, 1.167 mol) in ethanol (2 L) were heated at reflux overnight. The reaction solution was evaporated in vacuo and the residue purified by column chromatography to yield ethyl 3-(3-t-butyl-5-amino-1H-pyrazol-1-yl)benzoate (Example SS, 116 g, 40%) as a white solid together with 3-(5-amino-3-t-butyl-1H-pyrazol-1-yl)benzoic acid (93 g, 36%). ¹H NMR (DMSO-d₆): δ 8.09 (s, 1H), 8.05 (brd, J=8.0 Hz, 1H), 7.87 (brd, J=8.0 Hz, 1H), 7.71 (t, J=8.0 Hz, 1H), 5.64 (s, 1H), 4.35 (q, J=7.2 Hz, 2H), 1.34 (t, J=7.2 Hz, 3H), 1.28 (s, 9H).

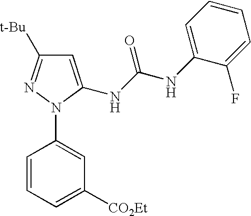

### Example 199

To a solution of Example SS (143 mg, 0.5 mmol) and Et₃N (143 mg, 0.5 mmol) in anhydrous THF (5 mL) was added 1-fluoro-2-isocyanato-benzene (67 mg, 0.5 mmol) at 0° C. The mixture was stirred at RT for 3 h, then poured into water (10 mL) and extracted with CH₂Cl₂. The combined organic extracts were washed with brine, dried (Na₂SO₄), filtered, concentrated and purified via preparative-TLC to afford ethyl 3-(3-t-butyl-5-[3-(2-fluorophenyl)ureido]-1H-pyrazol-1-yl)benzoate (40 mg, 19% yield).

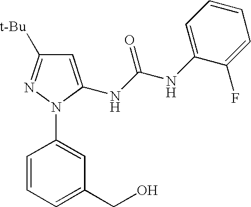

### Example 200

To a stirred solution of Example 199 (35 mg, 0.083 mmol) in THF (5 mL) was added LAH powder (7 mg, 0.18 mmol) by portions at 0° C. under N₂. The mixture was stirred at RT for 2 h, then quenched with water, and extracted with EtOAc. The combined organic extracts were washed with brine, dried (Na₂SO₄), filtered, concentrated and purified via preparative-TLC to afford 1-{3-t-butyl-1-[3-(hydroxymethyl)phenyl]-1H-pyrazol-5-yl}-3-(2-fluorophenyl)urea (20 mg, 63% yield).

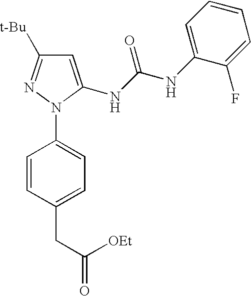

### Example 201

To a solution of Example RR (150 mg, 0.5 mmol) and Et₃N (101 mg, 1.0 mmol) in anhydrous THF (5 mL) was added 1-fluoro-2-isocyanato-benzene (68 mg, 0.5 mmol) at 0° C. The mixture was stirred at RT for 3 h before, then poured into water (50 mL), and extracted with CH₂Cl₂ (3×50 mL). The combined organic layers were washed with brine, dried (Na₂SO₄), filtered and concentrated to a solid, which was purified by column chromatography to afford 2-(4-(3-t-butyl-5-(3-(2-fluorophenyl)ureido)-1H-pyrazol-1-yl)-phenyl)acetate (140 mg, 64% yield).

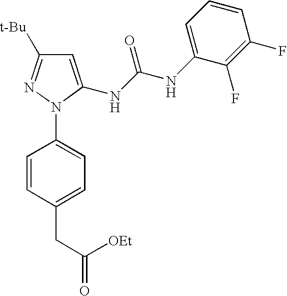

### Example 202

A solution of Example RR (300 mg, 1.0 mmol), Et₃N (202 mg, 2.0 mmol) and CDI (162 mg, 1.0 mmol) in DMF (5.0 mL) was stirred at RT for 6 h. The mixture was added 2,3-difluoro-aniline (129 mg, 1.0 mmol), stirred for 5 h, poured into water (50 mL) and extracted with CH₂Cl₂ (3×50 mL). The combined organic layers were washed with 1.0 N HCl, brine, dried (Na₂SO₄), filtered and concentrated to a solid, which was purified by column chromatography to afford ethyl 2-(4-(3-t-butyl-5-(3-(2,3-difluorophenyl)ureido)-1H-pyrazol-1-yl)phenyl)acetate (220 mg, 48% yield).

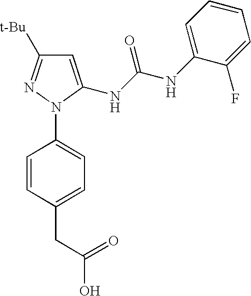

### Example 203

A mixture of Example 201 (100 mg, 0.22 mmol) in an aqueous solution of LiOH (2 N, 5 mL) and THF (10 mL) was stirred overnight at RT. After removal of the organic solvent, the mixture was extracted with Et₂O. The aqueous layer was then acidified with 2 N HCl to pH 4 and extracted with EtOAc. The combined organic layers were washed with brine, dried (Na₂SO₄), filtered and concentrated to a solid and dried to give the crude product, which was purified by reverse phase chromatography to afford 2-(4-(3-t-butyl-5-(3-(2-fluorophenyl)ureido)-1H-pyrazol-1-yl)phenyl)acetic acid (50 mg, 61% yield). ¹H NMR (400 MHz, DMSO-d₆): δ 10.07 (br s, 1H), 9.92 (br s, 1H), 7.91 (t, J=5.7 Hz, 1H), 7.38 (d, J=5.7 Hz, 2H), 7.30 (d, J=5.7 Hz, 2H), 7.14 (t, J=5.4 Hz, 1H), 7.05 (t, J=5.4 Hz, 1H), 6.96 (m, 1H), 6.25 (s, 1H), 3.26 (s, 2H), 1.24 (s, 9H); MS (ESI) m/z: 411 (M+H⁺).

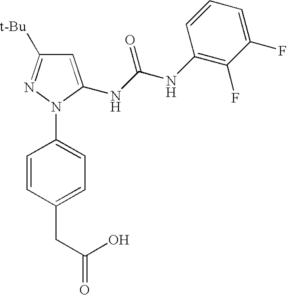

### Example 204

Using the same procedure as for Example 203, Example 202 (100 mg, 0.22 mmol) was transformed to afford 2-(4-(3-t-butyl-5-(3-(2,3-difluorophenyl)ureido)-1H-pyrazol-1-yl)-phenyl)acetic acid (50 mg, 53% yield). ¹H NMR (400 MHz, DMSO-d₆): δ 10.75 (br s, 1H), 7.62 (t, J=7.8 Hz, 1H), 7.43 (d, J=6.0 Hz, 2H), 7.28 (d, J=6.0 Hz, 2H), 7.04-6.95 (m, 2H), 6.22 (s, 1H), 3.28 (s, 2H), 1.24 (s, 9H); MS (ESI) m/z: 429 (M+H⁺).

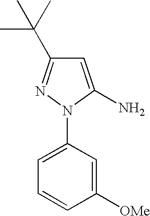

### Example TT

To a solution of 3-methoxyphenylhydrazine hydrochloride (1.0 g, 5.7 mmol) in PhMe (5 mL) was added pivaloylacetonitrile (0.70 g, 5.5 mmol). The reaction mixture was heated to reflux for 5 h, filtered and washed with hexane to yield 3-t-butyl-1-(3-methoxyphenyl)-1H-pyrazol-5-amine (1.22 g, 0.89% yield) as its hydrochloride salt as a pale yellow solid which was used without further purification. ¹H NMR (CDCl₃): δ 7.35 (t, J=8.4 Hz, 1H), 7.04 (t, J=2.1 Hz, 1H), 7.00 (dd, J=1.5 and 7.5 Hz, 1H), 6.95 (dd, J=2.1 and 8.4 Hz, 1H), 5.90 (bs, 2H), 5.83 (s, 1H), 3.81 (s, 3H), 1.89 (s, 9H); MS (EI) m/z: 246 (M+H⁺).

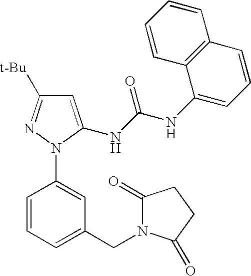

### Example 205

To a mixture of Example A1 (100 mg, 0.23 mmol), K₂CO₃ (64 mg, 0.46 mmol) and KI (10 mg) in DMF (2 mL) was added pyrrolidine-2,5-dione (23 mg, 0.23 mmol) at RT. The resulting mixture was stirred overnight, concentrated and purified by column chromatography to yield 1-(3-t-butyl-1-{3-[(2,5-dioxopyrrolidin-1-yl)methyl]phenyl}-1H-pyrazol-5-yl)-3-(naphthalen-1-yl)urea (50 mg, 44% yield). ¹H-NMR (300 MHz, DMSO-d₆): δ 9.00 (s, 1H), 8.86 (s, 1H), 8.02 (d, J=8.1 Hz, 1H), 7.89-7.92 (m, 2H), 7.63 (d, J=7.8 Hz, 1H), 7.42-7.55 (m, 6H), 7.29 (m, 1H), 6.40 (s, 1H), 4.62 (s, 2H), 2.63 (s, 2H), 1.27 (s, 9H).

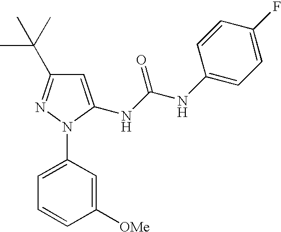

### Example 206

Using the same procedure as for Example 201, Example TT (70 mg, 0.29 mmol) and 4-fluorophenyl isocyanate (39 mg, 0.29 mmol) were combined to afford 1-(3-t-butyl-1-(3-methoxyphenyl)-1H-pyrazol-5-yl)-3-(4-fluorophenyl)urea as a white powder (38 mg, 35% yield). ¹H NMR (CDCl₃): δ 7.59 (bs, 1H), 7.16 (t, J=8.4 Hz, 1H), 6.8-7.1 (m, 8H), 6.77 (dd, J=1.8 and 8.7 Hz, 1H), 6.30 (s, 1H), 3.66 (s, 3H), 1.27 (s, 9H); MS (EI) m/z: 383 (M+H⁺.

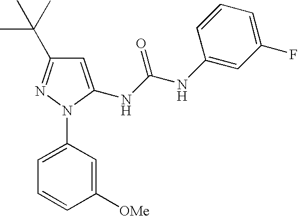

### Example 207

Using the same procedure as for Example 201, Example TT (60 mg, 0.21 mmol) and 3-fluorophenyl isocyanate (29 mg, 0.21 mmol) were combined to afford 1-(3-t-butyl-1-(3-methoxyphenyl)-1H-pyrazol-5-yl)-3-(3-fluorophenyl)urea (49 mg, 60% yield). ¹H NMR (CDCl₃): δ 7.2-7.3 (m, 3H), 7.17 (bs, 1H), 6.95-7.05 (m, 2H), 6.93 (dd, J=1.6, and 8.2 Hz, 1H), 6.87 (dd, J=1.8, and 7.6 Hz, 1H), 6.79 (dt, J=1.9, and 8.8 Hz, 1H), 6.64 (s, 1H), 6.39 (s, 1H), 3.77 (s, 3H), 1.35 (s, 9H); MS (EI) m/z: 383 (M+H⁺).

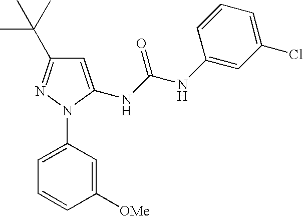

### Example 208

Using the same procedure as for Example 201, Example TT (70 mg, 0.29 mmol) and 3-chlorophenyl isocyanate (44 mg, 0.29 mmol) were combined to afford 1-(3-t-butyl-1-(3-methoxyphenyl)-1H-pyrazol-5-yl)-3-(3-chlorophenyl)urea (83 mg, 73% yield). ¹H NMR (CDCl₃): δ 8.30 (s, 1H), 7.38 (s, 1H), 7.20 (t, J=1.8 Hz, 1H), 7.07 (m, 2H), 6.95 (dt, J=1.2, and 7.8 Hz, 2H), 6.82 (t, J=2.1 Hz, 1H), 6.78 (s, 1H), 7.72 (dd, J=2.1, and 8.7 Hz, 1H), 6.28 (s, 1H), 3.56 (s, 3H), 1.21 (s, 9H); MS (EI) m/z: 399 (M+H⁺).

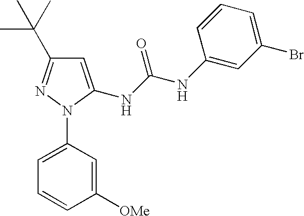

### Example 209

Using the same procedure as for Example 201, Example TT (70 mg, 0.29 mmol) and 3-bromophenyl isocyanate (57 mg, 0.29 mmol) were combined to afford 1-(3-t-butyl-1-(3-methoxyphenyl)-1H-pyrazol-5-yl)-3-(3-bromophenyl)urea as a white solid (107 mg, 85% yield). ¹H NMR (CDCl₃): δ 8.08 (bs, 1H), 7.38 (s, 1H), 7.23 (s, 1H). 7.0-7.2 (m, 4H), 7.8-7.9 (m, 2H), 6.75 (dd, J=2.4 and 8.4 Hz, 1H), 6.32 (s, 1H), 3.59 (s, 3H), 1.24 (s, 9H); MS (EI) m/z: 443 and 445 (M⁺ and M⁺+2).

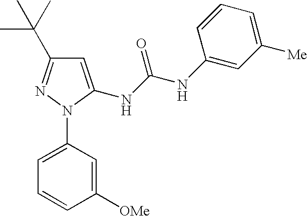

### Example 210

Using the same procedure as for Example 201, Example TT (70 mg, 0.29 mmol) and 3-methylphenyl isocyanate (38 mg, 0.29 mmol) were combined to afford 1-(3-t-butyl-1-(3-methoxyphenyl)-1H-pyrazol-5-yl)-3-m-tolylurea as a white solid (107 mg, 98% Yield). ¹H NMR (CDCl₃): δ 7.88 (bs, 1H), 7.34 (s, 1H), 7.0-7.2 (m, 2H), 6.95 (s, 1H), 6.8-6.94 (m, 4H). 6.73 (dd, J=2.4 and 8.4 Hz, 1H), 6.30 (s, 1H), 3.58 (s, 3H), 2.19 (s, 3H), 1.25 (s, 9H); MS (EI) m/z: 379 (M+H⁺).

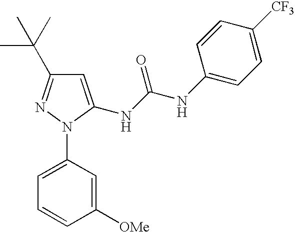

### Example 211

Using the same procedure as for Example 201, Example TT (70 mg, 0.29 mmol)) and 4-(trifluoromethyl)phenyl isocyanate (53 mg, 0.29 mmol) were combined to afford 1-(3-t-butyl-1-(3-methoxyphenyl)-1H-pyrazol-5-yl)-3-(4-(trifluoromethyl)phenyl)urea (73 mg, 59% yield). ¹H NMR (CDCl₃): δ 8.50 (s, 1H), 7.44 (AB quartet, J=8.7 Hz, 2H), 7.33 (s, 1H), 7.27 (AB quartet, J=8.7 Hz, 2H), 7.06 (t, J=7.8 Hz, 1H), 6.7-6.9 (m, 3H), 6.34 (s, 1H), 3.54 (s, 3H), 1.22 (s, 9H); MS (EI) m/z: 433 (M+H⁺).

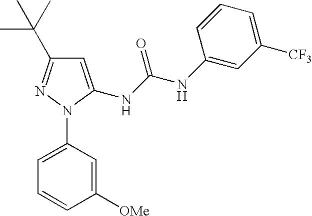

### Example 212

Using the same procedure as for Example 201, Example TT (50 mg, 0.20 mmol) and 3-(trifluoromethyl)phenyl isocyanate (30 mmg, 0.20 mmol) were combined to afford 1-(3-t-butyl-1-(3-methoxyphenyl)-1H-pyrazol-5-yl)-3-(3-(trifluoromethyl)phenyl)urea (30 mg, 39% yield). ¹H NMR (CDCl₃): δ 8.14 (s, 1H), 7.51 (s, 1H), 7.38 (d, J=8.1 Hz, 1H), 7.32 (t, J=8.0 Hz, 1H), 7.27 (d, J=7.6 Hz, 1H), 7.1-7.2 (m, 2H), 6.88 (t, J=2.0 Hz, 1H), 6.84 (dd, J=1.0 Hz, and 7.8 Hz, 1H), 6.79 (dd, J=2.4, and 7.8 Hz, 1H), 6.38 (s, 1H), 3.61 (s, 3H), 1.27 (s, 9H); MS (EI) m/z: 433 (M+H⁺).

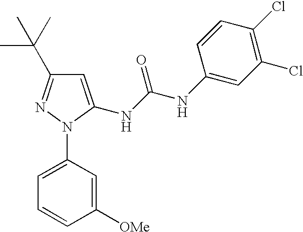

### Example 213

Using the same procedure as for Example 201, Example TT (70 mg, 0.29 mmol) and 3-chloro-4-(trifluoromethyl)phenyl isocyanate (63 mg, 0.29 mmol) were combined to afford 1-(3-t-butyl-1-(3-methoxyphenyl)-1H-pyrazol-5-yl)-3-(4-chloro-3-(trifluoromethyl)phenyl)urea as a white solid (49 mg, 37% Yield). ¹H NMR (CDCl₃): δ 8.48 (s, 1H), 7.52 (d, J=2.1 Hz, 1H), 7.38 (dd, J=2.1, 8.7 Hz, 1H), 6.79 (bs, 2H), 6.76 (s, 1H), 6.37 (s, 1H), 3.58 (s, 3H), 1.22 (s, 9H); MS (EI) m/z: 467 (M+H⁺)

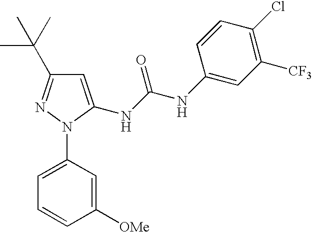

### Example 214

Using the same procedure as for Example 201, Example TT (70 mg, 0.29 mmol) and 3,4-dichlorophenyl isocyanate (54 mg, 0.29 mmol) were combined to afford 1-(3-t-butyl-1-(3-methoxyphenyl)-1H-pyrazol-5-yl)-3-(3,4-dichlorophenyl)urea (38 mg, 31% yield). ¹H NMR (CDCl₃): δ 8.13 (s, 1H), 7.35 (d, J=2.4 Hz, 1H), 7.24 (dd, J=0.6, and 3.3 Hz, 1H), 7.19 (s, 1H), 7.12 (t, J=8.1 Hz, 1H), 6.96 (dd, J=2.4, and 8.7 Hz, 1H), 6.7-6.9 (m, 3H), 6.37 (s, 1H), 3.62 (s, 3H), 1.24 (s, 9H); MS (EI) m/z: 433 (M+H⁺).

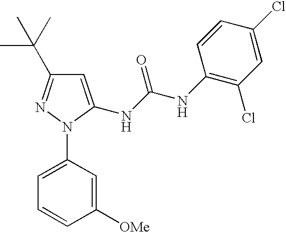

### Example 215

Using the same procedure as for Example 201, Example TT (70 mg, 0.29 mmol) and 2,4-dichlorophenyl isocyanate (54 mg, 0.29 mmol) were combined to afford 1-(3-t-butyl-1-(3-methoxyphenyl)-1H-pyrazol-5-yl)-3-(2,4-dichlorophenyl)urea (76 mg, 61% yield). ¹H NMR (CDCl₃): δ 7.96 (d, J=9.0 Hz), 7.67 (s, 1H), 7.65 (s, 1H), 7.29 (d, J=2.4 Hz, 1H), 7.19 (t, J=7.8 Hz, 1H), 7.14 (dd, J=2.4, and 9.0 Hz, 1H), 6.9-7.0 (m, 2H), 6.78 (dd, J=2.4, and 8.7 Hz, 1H), 6.33 (s, 1H), 3.70 (s, 3H), 1.32 (s, 9H); MS (EI) m/z: 433 (M+H⁺).

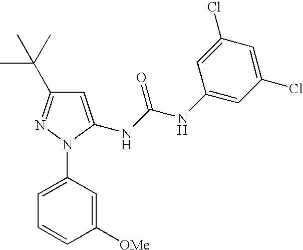

### Example 216

Using the same procedure as for Example 201, Example TT (70 mg, 0.29 mmol) and 3,5-dichlorophenyl isocyanate (54 mg, 0.29 mmol) were combined to afford 1-(3-t-butyl-1-(3-methoxyphenyl)-1H-pyrazol-5-yl)-3-(3,5-dichlorophenyl)urea (59 mg, 48% yield). ¹H NMR (CDCl₃): δ 7.73 (s, 1H), 7.1-7.3 (m, 3H), 7.03 (t, J=1.8 Hz, 1H), 6.9-7.0 (m, 3H), 6.84 (dd, J=1.8, and 7.5 Hz, 1H), 6.40 (s, 1H), 3.71 (s, 3H), 1.30 (s, 9H); MS (EI) m/z: 433 (M+H⁺).

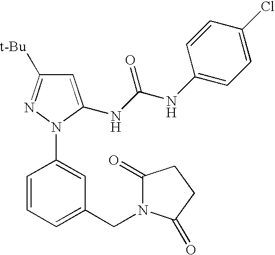

### Example 217

Using the same procedure as for Example 205, Example A2 (100.0 mg, 0.25 mmol) was transformed to afford 1-(3-t-butyl-1-{3-[(2,5-dioxopyrrolidin-1-yl)methyl]phenyl)-}1-pyrazol-5-yl)-3-(4-chlorophenyl)urea (35 mg, 29% yield). ¹H NMR (300 MHz, DMSO-d₆): δ 9.01 (s, 1H), 8.46 (s, 1H), 7.35-7.45 (m, 5H), 7.25-7.30 (m, 2H), 6.34 (s, 1H), 4.60 (s, 2H), 2.64 (s, 2H), 1.27 (s, 9H).

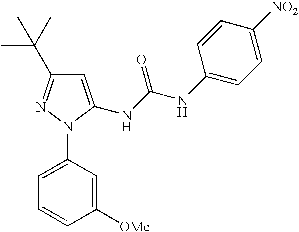

### Example 218

Using the same procedure as for Example 205, Example TT (70 mg, 0.29 mmol) and 4-nitrophenylisocyanate (47 mg, 0.29 mmol) were combined to afford 1-(3-t-butyl-1-(3-methoxyphenyl)-1H-pyrazol-5-yl)-3-(4-nitrophenyl)urea (62 mg, 53% yield). ¹H NMR (CDCl₃): δ 8.54 (s, 1H), 8.08 (AB quartet, J=9.0 Hz, 2H), 7.45 (AB quartet, J=9.0 Hz, 2H), 7.38 (s, 1H), 7.11 (t, J=8.1 Hz, 1H), 6.7-6.9 (m, 3H), 6.45 (s, 1H), 3.61 (s, 3H), 1.26 (s, 9H); MS (EI) m/z: 410 (M+H⁺).

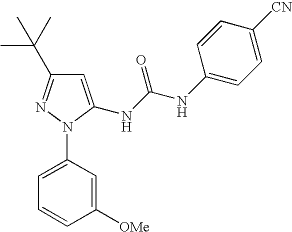

### Example 219

Using the same procedure as for Example 205, Example TT (70 mg, 0.29 mmol) and 4-cyanophenyl isocyanate (41 mg, 0.29 mmol) were combined to afford 1-(3-t-butyl-1-(3-methoxyphenyl)-1H-pyrazol-5-yl)-3-(4-cyanophenyl)urea (79 mg, 71% yield). ¹H NMR (CDCl₃): δ 8.70 (s, 1H), 7.47 (AB quartet, J=8.7 Hz, 2H), 7.40 (AB quartet, J=8.7 Hz, 2H), 7.37 (s, 1H), 7.11 (t, J=7.8 Hz, 1H), 6.7-6.9 (m, 3H), 6.42 (s, 1H), 3.59 (s, 3H), 1.24 (s, 9H); MS (EI) m/z: 390 (M+H⁺).

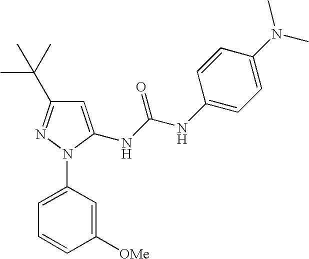

### Example 220

Using the same procedure as for Example 205, Example TT (70 mg, 0.29 mmol) and 4-(N,N-dimethylamino)phenyl isocyanate (46 mg, 0.29 mmol) were combined to afford 1-(3-t-butyl-1-(3-methoxyphenyl)-1H-pyrazol-5-yl)-3-(4-(dimethylamino)phenyl)urea as a brown oil (25 mg, 21% Yield). ¹H NMR (CDCl₃): δ 7.19 (t, J=8.1 Hz, 1H), 7.01 (AB quartet, J=9.0 Hz, 2H), 6.85-6.95 (m, 3H), 7.47 (dd, J=2.1, and 8.1 Hz, 1H), 6.60 (AB quartet, J=9.0 Hz, 2H), 6.40 (s, 1H), 3.73 (s, 3H), 2.92 (s, 6H), 1.32 (s, 9H); MS (EI) m/z: 408 (M+H

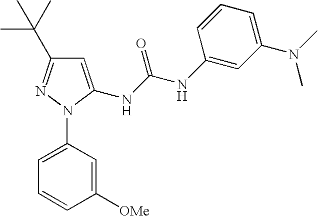

### Example 221

Using the same procedure as for Example 205, Example TT (62 mg, 0.25 mmol) and 3-(N,N-dimethylamino)phenyl isocyanate (52 mg, 0.32 mmol) were combined to afford 1-(3-t-butyl-1-(3-methoxyphenyl)-1H-pyrazol-5-yl)-3-(3-(dimethylamino)phenyl)urea (11 mg, 11% yield). ¹H NMR (CDCl₃): δ 7.24 (t, J=8.2 Hz, 1H), 7.11 (t, J=8.1 Hz, 1H), 6.9-7.0 (m, 4H), 6.83 (m, 1H), 6.66 (bs, 1H), 6.48 (dt, J=2.4, and 8.2 Hz, 2H), 6.41 (s, 1H), 3.74 (s, 3H), 2.89 (s, 6H), 1.34 (s, 9H); MS (EI) m/z: 408 (M+H⁺).

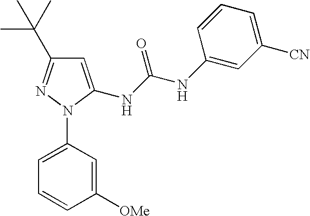

### Example 222

Using the same procedure as for Example 205, Example TT (45 mg, 0.18 mmol) and 3-cyanophenyl isocyanate (26 mg, 0.18 mmol) were combined to afford 1-(3-t-butyl-1-(3-methoxyphenyl)-1H-pyrazol-5-yl)-3-(3-cyanophenyl)urea (35 mg, 50% yield). ¹H NMR (CDCl₃): δ 8.14 (s, 1H), 7.61 (s, 1H), 7.52 (m, 1H), 7.35 (t, J=8.0 Hz, 1H), 7.29 (d, J=6.9 Hz, 1H), 7.21 (d, J=8.0 Hz, 1H), 7.18 (s, 1H), 6.90 (s, 1H), 6.88 (d, J=7.6 Hz, 1H), 6.80 (dd, J=2.4, and 7.6 Hz, 1H), 6.42 (s, 1H), 3.67 (s, 3H), 1.30 (s, 9H); MS (EI) m/z: 390 (M+H⁺).

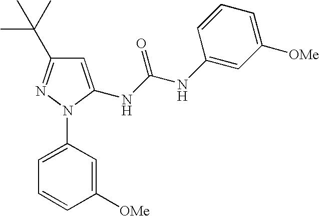

### Example 223

Using the same procedure as for Example 205, Example TT (45 mg, 0.18 mmol) and 3-methoxyphenyl isocyanate (26 mg, 0.18 mmol) were combined to afford 1-(3-t-butyl-1-(3-methoxyphenyl)-1H-pyrazol-5-yl)-3-(3-methoxyphenyl)urea (17 mg, 24% yield). ¹H NMR (CDCl₃): δ 7.28 (s, 1H), 7.24 (t, J=8.0 Hz, 1H), 7.15 (t, J=8.2 Hz, 1H), 6.9-7.0 (m, 4H), 6.83 (dd, J=2.3, and 8.7 Hz, 1H), 6.71 (dd, J=1.6, and 8.0 Hz, 1H), 6.64 (dd, J=2.4, and 8.2 Hz, 1H), 6.39 (s, 1H), 3.74 (s, 3H), 3.72 (s, 3H), 1.33 (s, 9H); MS (EI) m/z: 395 (M+H⁺).

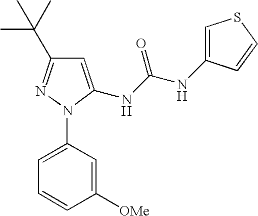

### Example 224

Using the same procedure as for Example 205, Example TT (70 mg, 0.29 mmol) and 3-thienyl isocyanate (36 mg, 0.29 mmol) were combined to afford 1-(3-t-butyl-1-(3-methoxyphenyl)-1H-pyrazol-5-yl)-3-(thiophen-3-yl)urea (45 mg, 43% yield). ¹H NMR (CDCl₃): δ 7.05-7.3 (m, 4H), 6.8-7.0 (m, 4H), 6.76 (s, 1H), 6.40 (s, 1H), 3.76 (s, 3H), 1.35 (s, 9H); MS (EI) m/z: 371 (M+H⁺).

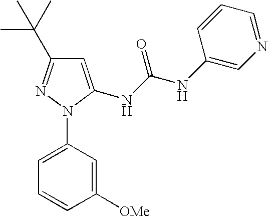

### Example 225

Using the same procedure as for Example 205, Example TT (86 mg, 0.35 mmol) and 3-pyridinylisocyanate (51 mg, 0.43 mmol) were combined to afford 1-(3-t-butyl-1-(3-methoxyphenyl)-1H-pyrazol-5-yl)-3-(pyridin-3-yl)urea as a white solid (89 mg, 69% yield). ¹H NMR (DMSO-d₆): δ 10.0 (bs, 1H), 8.92 (bs, 1H), 8.87 (s, 1H), 8.39 (d, J=5.2 Hz, 1H), 8.17 (d, J=8.4 Hz, 1H), 7.70 (dd, J=5.1, and 8.1 Hz, 1H), 7.41 (t, J=8.2 Hz, 1H), 7.0-7.1 (m, 2H), 6.96 (dd, J=2.4, and 8.3 Hz, 1H), 6.38 (s, 1H), 3.80 (s, 3H), 1.29 (s, 9H); MS (EI) m/z: 366 (M+H⁺).

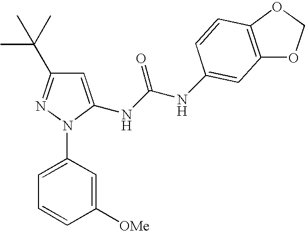

### Example 226

Using the same procedure as for Example 205, Example TT (86 mg, 0.35 mmol) and 5-isocyanatobenzo[d][1,3]dioxole (69 mg, 0.43 mmol) were combined to afford 1-(benzo[d][1,3]dioxo-5-yl)-3-(3-t-butyl-1-(3-methoxyphenyl)-1H-pyrazol-5-yl)urea as a pale yellow solid (98 mg, 68% yield). ¹H NMR (DMSO-d₆): δ 8.94 (s, 1H), 8.92 (bs, 1H), 8.31 (s, 1H), 7.42 (t, J=8.1 Hz, 1H), 7.0-7.2 (m, 3H), 6.98 (dd, J=1.8, and 8.4 Hz, 1H), 6.80 (d, J=8.4 Hz, 1H), 6.71 (dd, J=2.0, and 8.4 Hz, 1H), 6.35 (s, 1H), 5.96 (s, 2H), 3.80 (s, 3H), 1.28 (s, 9H); MS (EI) m/z: 409 (M+H⁺).

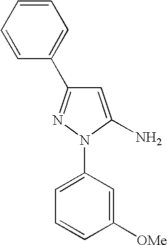

### Example UU

To a solution of 3-methoxyphenylhydrazine hydrochloride (0.6 g, 3.44 mmol) in toluene was added commercially available benzoyl acetonitrile (0.5 g, 3.44 mmol). The reaction mixture was heated to reflux overnight, filtered and washed with hexane to obtain 1-(3-methoxyphenyl)-3-phenyl-1H-pyrazol-5-amine (0.82 g, 79% yield) as a grey hydrochloride salt which was used without any further purification. ¹H NMR (DMSO-d₆): δ 7.78 (m, 2H), 7.2-7.6 (m, 6H), 6.97 (m, 1H), 6.01 (s, 1H), 3.81 (s, 3H), 1.27 (s, 9H); MS (EI) m/z: 266 (M+H⁺).

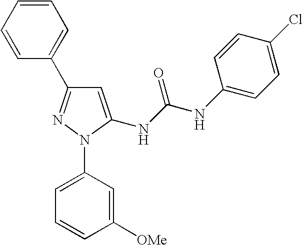

### Example 227

Using the same procedure as for Example 205, Example UU (70 mg, 0.23 mmol) and 4-chlorophenylisocyanate (36 mg, 0.23 mmol) were combined to afford 1-(4-chlorophenyl)-(3-methoxyphenyl)-3-phenyl-1H-pyrazol-5-yl)urea (75 mg, 77% yield). ¹H NMR (DMSO-d₆): 58.59 (s, 1H), 7.86 (d, J=1.6 Hz, 1H), 7.84 (s, 1H), 7.3-7.5 (m, 9H), 7.21 (s, 1H), 7.19 (d, J=1.6 Hz, 1H), 7.05 (dd, J=2.0, and 9.2 Hz, 1H), 6.94 (s, 1H), 3.83 (s, 3H); MS (EI) m/z: 419 (M+H⁺).

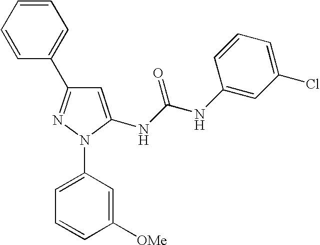

### Example 228

Using the same procedure as for Example 205, Example UU (50 mg, 0.17 mmol) and 3-chlorophenylisocyanate (25 mg, 0.17 mmol) were combined to afford 1-(3-chlorophenyl)-(3-methoxyphenyl)-3-phenyl-1H-pyrazol-5-yl)urea (46 mg, 66% yield). ¹H NMR (CDCl₃): δ7.92 (s, 1H), 7.67 (dd, J=1.5, and 8.2 Hz, 2H), 7.54 (s, 1H), 7.25-7.4 (m, 3H), 7.15 (t, J=2.0 Hz, 1H), 7.09 (t, J=8.1 Hz, 1H), 7.02 (t, J=8.0 Hz, 1H), 6.8-7.0 (m, 3H), 6.83 (dd, J=1.2, and 7.8 Hz, 1H), 6.71 (dd, J=2.0, and 8.1 Hz, 1H), 6.64 (s, 1H), 3.57 (s, 3H); MS (EI) m/z: 419 (M+H⁺).

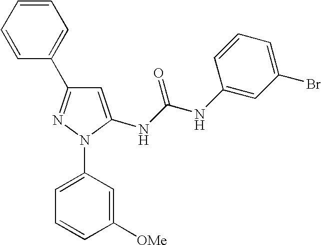

### Example 229

Using the same procedure as for Example 205, Example UU (50 mg, 0.17 mmol) and 3-bromophenylisocyanate (25 mg, 0.17 mmol) were combined to afford 1-(3-bromophenyl)-(3-methoxyphenyl)-3-phenyl-1H-pyrazol-5-yl)urea (46 mg, 60% yield). ¹H NMR (DMSO-d₆): δ 9.28 (s, 1H), 8.62 (s, 1H), 7.85 (m, 3H), 7.0-7.5 (m, 10H), 6.95 (s, 1H), 3.83 (s, 3H); MS (EI) m/z: 463 and 465 (M⁺ and M⁺+2).

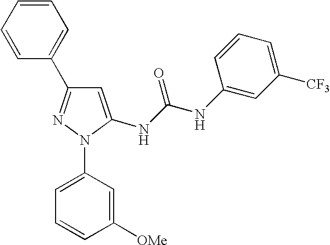

### Example 230

Using the same procedure as for Example 205, Example UU (50 mg, 0.17 mmol) and 3-trifluoromethylphenyl isocyanate (31 mg, 0.17 mmol) were combined to afford 1-(1-(3-methoxyphenyl)-3-phenyl-1H-pyrazol-5-yl)-3-(3-trifluoromethyl)phenyl)urea (43 mg, 57% yield). ¹H NMR (DMSO-d₆): δ 9.45 (s, 1H), 8.67 (s, 1H), 8.00 (s, 1H), 7.87 (m, 2H), 7.0-7.6 (m, 10H), 6.97 (s, 1H), 3.83 (s, 3H); MS (EI) m/z: 453 (M+H⁺).

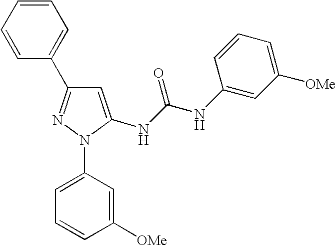

### Example 231

Using the same procedure as for Example 205, Example UU (50 mg, 0.17 mmol) and 3-methoxyphenyl isocyanate (25 mg, 0.17 mmol) were combined to afford 1-(3-methoxyphenyl)-3-(1-(3-methoxyphenyl)-3-phenyl-1H-pyrazol-5-yl)urea (47 mg, 68% yield). ¹H NMR (DMSO-d₆): [symbol] 9.11 (s, 1H), 8.52 (s, 1H), 7.86 (d, J=1.3 Hz, 1H), 7.84 (s, 1H), 1.47 (t, J=7.8 Hz, 1H), 7.44 (t, J=7.8 Hz, 2H), 7.34 (t, J=7.3 Hz, 1H), 7.0-7.2 (m, 5H), 6.94 (s, 1H), 6.93 (m, 1H), 6.57 (dd, J=2.4, and 8.2 Hz, 1H), 3.83 (s, 3H), 3.72 (s, 3H); MS (EI) m/z: 415 (M+H⁺).

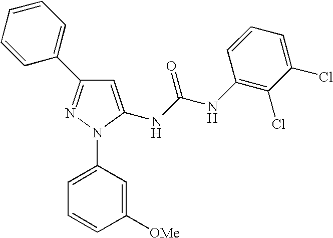

### Example 232

Using the same procedure for Example 205, Example UU (50 mg, 0.17 mmol) and 2,3-dichlorophenyl isocyanate (31 mg, 0.17 mmol) were combined to afford 1-(2,3-dichlorophenyl)-(3-methoxyphenyl)-3-phenyl-1H-pyrazol-5-yl)urea (41 mg, 55% yield). ¹H NMR (DMSO-d₆): δ 9.37 (s, 1H), 8.87 (s, 1H), 7.07 (dd, J=3.4, and 6.4 Hz, 1H), 7.86 (d, J=1.4 Hz, 1H), 7.84 (s, 1H), 7.50 (t, J=8.4 Hz, 1H), 7.44 (t, J=7.3 Hz, 2H), 7.2-7.4 (m, 5H), 7.06 (m, 1H), 6.95 (s, 1H), 3.84 (s, 3H); MS (EI) m/z: 453 (M+H⁺).

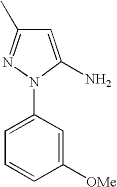

### Example VV

To a suspension of NaH (60%, 12.0 g, 0.3 mol) in THF (200 mL) was added dropwise acetic acid ethyl ester (17 g, 0.2 mol) and anhydrous acetonitrile (100 g, 0.24 mol) in THF (200 mL) at 80° C. The resulting mixture was refluxed overnight, and then cooled to RT. After removal of the volatiles in vacuo, the residue was diluted in EtOAc and aqueous 10% HCL. The combined organic extracts were washed with saturated NaHCO₃ and brine, then dried (MgSO₄), filtered, concentrated to yield 3-oxobutyronitrile (10 g), which was used for the next step reaction without further purification.

To a solution of 3-oxobutanenitrile (300 mg, 3.6 mmol) and 3-methoxyphenyl-hydrazine HCl (630 mg, 3.6 mmol) in absolute ethanol at RT was added conc. HCl (0.3 mL). The reaction mixture was stirred at 80° C. for 13 h. The solvent was evaporated under reduced pressure to obtain the crude product 1-(3-methoxyphenyl)-3-methyl-1H-pyrazol-5-amine as brown foam hydrochloride salt (690 mg, 80% yield), which was used without further purification. MS (EI) m/z: 204 (M+H⁺).

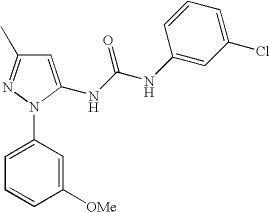

### Example 233

Using the same procedure as for Example 205, Example VV (60 mg, 0.25 mmol) and 3-chlorophenyl isocyanate (38 mg, 0.25 mmol) were combined to afford 1-(3-chlorophenyl)-3-(1-(3-methoxyphenyl)-3-methyl-1H-pyrazol-5-yl)urea (15 mg, 17% yield). ¹H NMR (CDCl₃): δ 7.97 (bs, 1H), 7.34 (bs, 1H), 7.30 (t, J=2.0 Hz, 1H), 7.0-7.25 (m, 4H), 6.85 (s, 1H), 6.84 (m, 1H), 6.79 (m, 1H), 6.30 (s, 1H), 3.67 (s, 3H), 2.22 (s, 3H); MS (EI) m/z: 357 (M+H⁺).

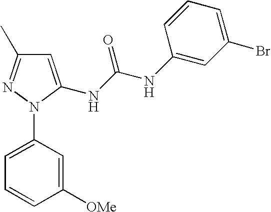

### Example 234

Using the same procedure as for Example 205, Example VV (50 mg, 0.21 mmol) and 3-bromophenyl isocyanate (41 mg, 0.21 mmol) were combined to afford 1-(3-bromophenyl)-3-(1-(3-methoxyphenyl)-3-methyl-1H-pyrazol-5-yl)urea (12 mg, 15% yield). ¹H NMR (CDCl₃): δ 8.20 (bs, 1H), 7.51 (bs, 1H), 7.40 (m, 2H), 7.0-7.2 (m, 4H), 6.7-6.8 (m, 3H), 6.27 (s, 1H), 3.63 (s, 3H), 2.18 (s, 3H); MS (EI) m/z: 401 and 403 (M⁺ and M⁺+2).

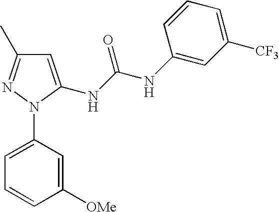

### Example 235

Using the same procedure as for Example 205, Example VV (50 mg, 0.21 mmol) and 3-(trifluoromethyl)phenyl isocyanate (39 mg, 0.21 mmol) were combined to afford 1-(1-(3-methoxyphenyl)-3-methyl-1H-pyrazol-5-yl)-3-(3-(trifluoromethyl)phenyl)urea (32 mg, 39% yield). ¹H NMR (DMSO-d₆): δ 8.46 (bs, 1H), 7.53 (bs, 1H), 7.49 (s, 1H), 7.2-7.4 (m, 3H), 7.13 (t, J=8.0 Hz), 6.7-6.8 (m, 3H), 6.29 (s, 1H), 3.60 (s, 3H), 2.15 (s, 3H); MS (EI) m/z: 357 (M+H⁺).

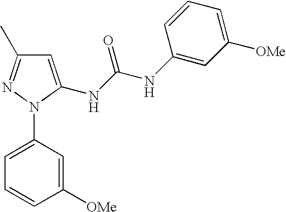

### Example 236

Using the same procedure as for Example 205, Example VV (50 mg, 0.21 mmol) and 3-methoxyphenyl isocyanate (30 mg, 0.21 mmol) were combined to afford 1-(3-methoxyphenyl)-3-(1-(3-methoxyphenyl)-3-methyl-1H-pyrazol-5-yl)urea (6 mg, 8% yield). ¹H NMR (CDCl₃): δ 7.2-7.4 (m, 1H), 7.17 (t, J=8.4 Hz, 1H), 6.99 (t, J=2.0 Hz, 1H), 6.9-7.0 (m, 2H), 6.86 (m, 1H), 6.76 (dd, J=1.2, and 8.0 Hz, 1H), 6.65 (dd, J=2.4, and 8.4 Hz, 1H), 6.34 (s, 1H), 3.78 (s, 3H), 3.75 (s, 3H), 2.28 (s, 3H); MS (EI)/z: 353 (M+H⁺).

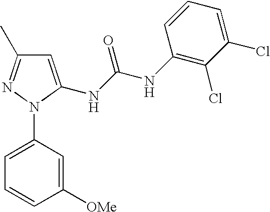

### Example 237

Using the same procedure as for Example 205, Example VV (50 mg, 0.21 mmol) and 2,3-dichlorophenyl isocyanate (39 mg, 0.21 mmol) were combined to afford 1-(2,3-dichlorophenyl)-3-(1-(3-methoxyphenyl)-3-methyl-1H-pyrazol-5-yl)urea (23 mg, 28% yield). ¹H NMR (CDCl₃): δ 8.08 (m, 1H), 7.60 (s, 1H), 7.32 (t, J=8.4 Hz, 1H), 7.19 (d, J=1.2 Hz, 1H), 7.18 (s, 1H), 7.01 (m, 2H), 6.97 (bs, 1H), 6.89 (dd, J=2.1, and 8.3 Hz, 1H), 6.35 (s, 1H), 3.79 (s, 3H), 2.35 (s, 3H); MS (EI) m/z: 391 (M+H⁺).

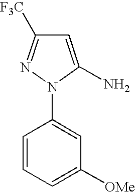

### Example WW

To a suspension of NaH (60% 6.0 g, 0.15 mol) in THF (100 ml) was added dropwise trifluoro-acetic acid ethyl ester (14.2 g, 0.1 mol) and anhydrous acetonitrile (50 g, 0.12 mol) in THF (100 ml) at 80° C. The resulting mixture was refluxed overnight, and then cooled to RT. After removal of the volatiles in vacuo, the residue was diluted in EtOAc and aqueous 10% HCL. The organic layer was washed with water and brine, dried (MgSO₄), filtered and concentrated to yield 15 g of the crude product, which was used for the next step reaction without further purification.

To a mixture of (3′-methoxyphenyl)-hydrazine (690 mg, 5.0 mmol) and commercially available 4,4,4-trifluoro-3-oxo-butyronitrile (822 mg, 6.0 mmol) in ethanol (50 mL) was added conc. HCl (5 mL). The resulting mixture was heated to reflux for 3 h. After removal of the solvent, the residue was washed with Et₂O to afford 0.95 g of the crude 3-(trifluoromethyl)-1-(3-methoxyphenyl)-1H-pyrazol-5-amine, which was used to the next reaction without further purification. MS (ESI) m/z: 258 (M+H⁺).

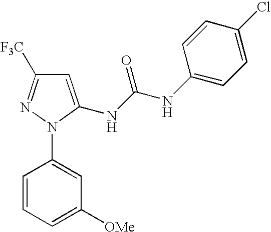

### Example 238

To a solution of Example WW (100 mg, 0.39 mmol) and Et₃N (80 mg, 0.8 mmol) in THF (30 mL) was added 1-chloro-4-isocyanato-benzene (153 mg, 1.0 mmol) at 0° C. in ice-water bath. The resulting mixture was stirred at 0° C. for 30 min and then warmed to RT for 3 h. The reaction mixture was quenched with 1.0 N HCl and extracted with CH₂Cl₂ (3×100 mL). The combined organic extracts were washed with brine, dried (Na₂SO₄), filtered and concentrated to the crude product, which was purified by preparative HPLC to afford 85 mg of 1-(4-chlorophenyl)-3-(3-(trifluoromethyl)-1-(3-methoxyphenyl)-1H-pyrazol-5-yl)urea. ¹H NMR (300 MHz, DMSO-d₆): δ 9.25 (s, 1H), 8.72 (s, 1H), 7.50 (t, J=6.3 Hz, 1H), 7.42 (d, J=6.6 Hz, 2H), 7.31 (d, J=6.6 Hz, 2H), 7.15-7.12 (m, 3H), 6.87 (s, 1H), 3.81 (s, 3H). MS (ESI) m/z: 411 (M+H⁺)

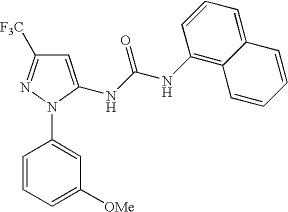

### Example 239

Using the same procedure as for Example 205, Example WW (100 mg, 0.39 mmol) and 1-Isocyanato-naphthalene (169 mg, 1.0 mmol) were combined to afford 70 mg of 1-(3-(trifluoromethyl)-1-(3-methoxyphenyl)-1H-pyrazol-5-yl)-3-(naphthalen-1-yl)urea. ¹H NMR (300 MHz, DMSO-d₆): [symbol] 9.16 (s, 1H), 9.08 (s, 1H), 7.95-7.85 (m, 3H), 7.66 (d, J=6.0 Hz, 1H), 7.55-7.41 (m, 4H), 7.22-7.12 (m, 3H), 6.88 (s, 1H), 3.83 (s, 3H). MS (ESI) m/z: 427 (M+H⁺)

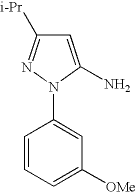

### Example XX

To a suspension of NaH (60%, 6.0 g, 0.15 mol) in THF (100 mL) was added dropwise isobutyric acid ethyl ester (11.6 g, 0.1 mol) and anhydrous acetonitrile (50 g, 0.12 mol) in THF (100 mL) at 80° C. The resulting mixture was refluxed overnight, then cooled to RT. After removal of the volatiles in vacuo, the residue was diluted in EtOAc and aqueous 10% HCL. The combined organic extracts were dried (Na₂SO₄), filtered, concentrated to yield 4-methyl-3-oxopentanenitrile (8.5 g), which was used for the next step reaction without further purification.

To a mixture of (3-methoxy-phenyl)-hydrazine (690 mg, 5.0 mmol) and 4-methyl-3-oxo-pentanenitrile (660 mg, 6.0 mmol) in ethanol (50 mL) was added conc. HCl (5 mL). The resulting mixture was heated to reflux for 3 h. After removal of the solvent, the residue was washed with Et₂O to afford 0.95 g of the crude 3-isopropyl-1-(3-methoxyphenyl)-1H-pyrazol-5-amine, which was used to the next reaction without further purification MS (ESI) m/z: 232 (M+H⁺).

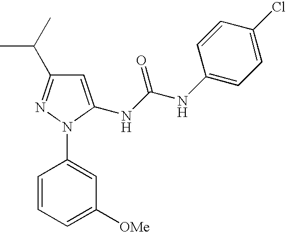

### Example 240

To a solution of Example XX (100 mg, 0.43 mmol) and Et₃N (80 mg, 0.8 mmol) in THF (30 mL) was added 1-chloro-4 isocyanato-benzene (153 mg, 1.0 mmol) at 0° C. in an ice-water bath. The resulting mixture was stirred at 0° C. for 30 min and then warmed to RT for 3 h. The reaction mixture was quenched with 1.0 N HCl and extracted with CH₂Cl₂ (3×100 mL). The combined organic extracts were washed with brine, dried ((Na₂SO₄), filtered and concentrated to the crude product, which was purified by preparative HPLC to afford 85 mg of 1-(4-chlorophenyl)-3-(3-isopropyl-1-(3-methoxyphenyl)-1H-pyrazol-5-yl)urea. ¹H NMR (300 MHz, DMSO-d₆): δ 9.08 (s, 1H), 9.04 (s, 1H), 7.45 (d, J=6.0 Hz, 2H), 7.41 (t, J=6.6 Hz, 1H), 7.30 (d, J=6.6 Hz, 2H), 7.05-6.98 (m, 3H), 6.36 (s, 1H), 3.78 (s, 3H). 1.12 (d, J=5.1 Hz, 6H), MS (ESI) m/z: 385 (M+H⁺)

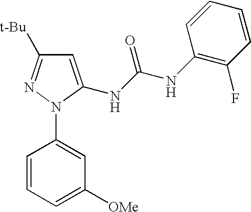

### Example 241

To a solution of Example TT (123 mg, 0.5 mmol) and Et3N (101 mg, 1.0 mmol) in anhydrous THF (5 mL) was added 1-fluoro-2-isocyanato-benzene (69 mg, 0.5 mmol) at 0° C. This resulted mixture was stirred at RT for 3 h, and extracted with EtOAc. The combined organic extracts were washed with brine, dried (Na₂SO₄), filtered, concentrated and purified by preparative TLC to afford 1-[3-t-butyl-1-(3-methoxy-phenyl)-1H-pyrazol-5-yl]-3-(2-fluorophenyl)urea. ¹H-NMR (300 MHz, DMSO-d₆): δ 8.92 (s, 1H), 8.80 (s, 1H), 8.06 (t, J=7.5 Hz, 1H), 7.39 (t, J=7.5 Hz, 1H), 7.17-6.96 (m, 6H), 6.35 (s, 1H), 3.75 (s, 3H), 1.22 (s, 9H); MS (ESI) m/z: 383 (M+H⁺).

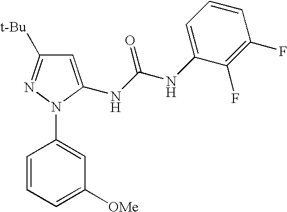

### Example 242

Using the same procedure as for Example 202, Example TT (123 mg, 0.5 mmol) and 2,3-difluoro-phenylamine (65 mg, 0.5 mm ol) were combined to afford 1-[3-t-butyl-1-(3-methoxy-phenyl)-1H-pyrazol-5-yl]-3-(2,3-difluorophenyl)urea. ¹H-NMR (300 MHz, DMSO-d₆): δ 9.11 (s, 1H), 8.84 (s, 1H), 7.87 (t, J=7.8 Hz, 1H), 7.40 (t, J=7.8 Hz, 1H), 7.09-6.94 (m, 5H), 6.36 (s, 1H), 3.76 (s, 3H), 1.23 (s, 9H); MS (ESI) m/z: 401 (M+H⁺).

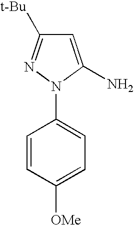

### Example YY

A mixture of (4-methoxy-phenyl)-hydrazine (17.4 g, 0.1 mol) and 4,4-dimethyl-3-oxo-pentanenitrile (13.8 g, 0.11 mol) in ethanol (500 mL) and conc. HCl (50 mL) was heated to reflux overnight. After removal of the solvent, the residue was purified by column chromatography to give 3-t-butyl-1-(4-methoxyphenyl)-1H-pyrazol-5-amine (20 g, 82% yield). ¹H-NMR (300 MHz, DMSO-d₆): δ 7.38 (d, J=9.0 Hz, 2H), 6.97 (d, J=9.0 Hz, 2H), 5.32 (s, 1H), 4.99 (br s, 2H), 3.75 (s, 3H), 1.17 (s, 9H); MS (ESI) m/z: 246 (M+H⁺).

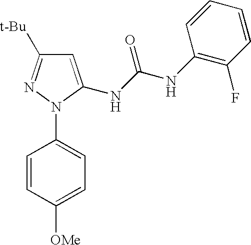

### Example 243

Using the same procedure as for Example 205, Example YY (123 mg, 0.5 mmol) and 1-fluoro-2-isocyanato-benzene (69 mg, 0.5 mmol) were combined to afford 1-[3-t-butyl-1-(4-methoxyphenyl)-1H-pyrazol-5-yl]-3-(2-fluorophenyl)urea. ¹H-NMR (300 MHz, DMSO-d₆): δ 9.01 (s, 1H), 8.89 (s, 1H), 8.09 (t, J=7.8 Hz, 1H), 7.36 (d, J=8.7 Hz, 2H), 7.09-7.21 (m, 2H), 7.05 (d, J=8.7 Hz, 2H), 6.97 (t, J=8.7 Hz, 1H), 6.32 (s, 1H), 3.79 (s, 3H), 1.23 (s, 9H); MS (ESI) m/z: 383 (M+H⁺).

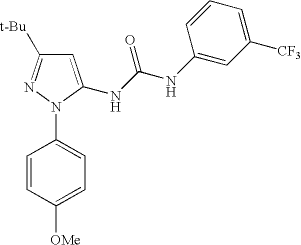

### Example 244

Using the same procedure as for Example 205, Example YY (123 mg, 0.5 mmol) and 1-isocyanato-3-trifluoromethyl-benzene (93 mg, 0.5 mmol) were combined to afford 1-[3-t-butyl-1-(4-methoxyphenyl)-1H-pyrazol-5-yl]-3-(3-trifluoromethylphenyl)urea (65 mg, 30% yield). ¹H-NMR (300 MHz, DMSO-d₆): δ 9.38 (s, 1H), 8.40 (s, 1H), 7.94 (br s, 1H), 7.45 (d, J=4.8 Hz, 2H), 7.38 (d, J=9.0 Hz, 2H), 7.27 (m, 1H), 7.03 (d, J=9.0 Hz, 2H), 6.32 (s, 1H), 3.78 (s, 3H), 1.24 (s, 9H); MS (ESI) m/z: 433 (M+H⁺).

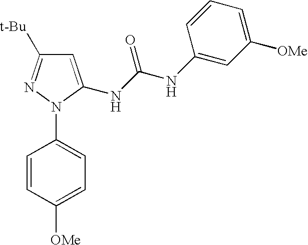

### Example 245

Using the same procedure as for Example 205, Example YY (123 mg, 0.5 mmol) and 1-Isocyanato-3-methoxy-benzene (93 mg, 0.5 mmol) were combined to afford 1-[3-t-butyl-1-(4-methoxyphenyl)-1H-pyrazol-5-yl]-3-(3-methoxy-phenyl)urea (65 mg, 33% yield). ¹H-NMR (300 MHz, DMSO-d₆): δ 9.98 (s, 1H), 8.25 (s, 1H), 7.37 (d, J=8.7 Hz, 2H), 7.13-7.03 (m, 4H), 6.82 (d, J=6.9 Hz, 1H), 6.52 (d, J=6.9 Hz, 1H), 6.31 (s, 1H), 3.78 (s, 3H), 1.23 (s, 9H); MS (ESI) m/z: 395 (M+H⁺).

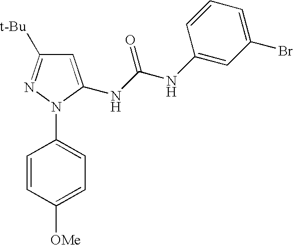

### Example 246

Using the same procedure as for Example 205, Example YY (123 mg, 0.5 mmol) and 1-bromo-3-isocyanato-benzene (98 mg, 0.5 mmol) were combined to afford 1-(3-bromophenyl)-3-[3-t-butyl-1-(4-methoxyphenyl)-1H-pyrazol-5-yl]urea (65 mg, 29% yield). ¹H-NMR (300 MHz, DMSO-d₆): δ 9.18 (s, 1H), 8.34 (s, 1H), 7.80 (br s, 1H), 7.37 (d, J=9.0 Hz, 2H), 7.18 (d, J=5.1 Hz, 2H), 7.12 (m, 1H), 7.03 (d, J=9.0 Hz, 2H), 6.31 (s, 1H), 3.78 (s, 3H), 1.24 (s, 9H); MS (ESI) m/z: 443 (M+H⁺).

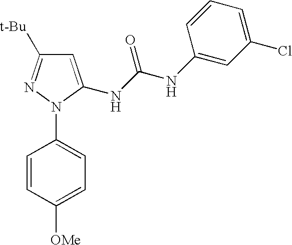

### Example 247

Using the same procedure as for Example 205, Example YY (123 mg, 0.5 mmol) and 1-chloro-3-isocyanato-benzene (76 mg, 0.5 mmol) were combined to afford 1-[3-t-butyl-1-(4-methoxyphenyl)-1H-pyrazol-5-yl]-3-(3-chlorophenyl)urea (65 mg, 33% yield). ¹H-NMR (300 MHz, DMSO-d₆): δ 9.17 (s, 1H), 8.34 (s, 1H), 7.65 (t, J=2.1 Hz, 1H), 7.37 (d, J=9.0 Hz, 2H), 7.22 (m, 1H), 7.15 (m, 1H), 6.31 (s, 1H), 3.78 (s, 3H), 1.24 (s, 9H); MS (ESI) m/z: 399 (M+H⁺).

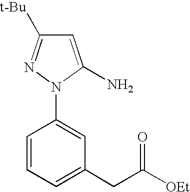

### Example ZZ

A mixture of 1-(3-nitrophenyl)ethanone (82.5 g, 0.5 mol), toluene-4-sulfonic acid (3 g) and sulfur (32 g, 1.0 mol) in morpholine (100 mL) was heated to reflux for 3 h. After removal of the solvent, the residue was dissolved in dioxane (100 mL). The mixture was added concentrated HCl (100 mL) and then heated to reflux for 5 h. After removal of the solvent, the residue was extracted with EtOAc (3×150 mL). The combined organic extracts were washed with brine, dried (Na₂SO₄), filtered, and concentrated. The residue was dissolved in ethanol (250 mL) and SOCl₂ (50 mL) and heated to reflux for 2 h. After removal of the solvent, the residue was extracted with EtOAc (3×150 mL). The combined organic extracts were washed with brine, dried (Na₂SO₄), filtered, concentrated and purified via column chromatography to afford ethyl (3-nitrophenyl)acetate (40 g). ¹H-NMR (300 MHz, DMSO-d₆): δ 8.17 (s, 1H), 8.11 (d, J=7.2 Hz, 1H), 7.72 (d, J=7.2 Hz, 1H), 7.61 (t, J=7.8 Hz, 1H), 4.08 (q, J=7.2 Hz, 2H), 3.87 (s, 2H), 1.17 (t, J=1.2 Hz, 3H)

A mixture of (3-nitrophenyl)acetic acid ethyl ester (21 g, 0.1 mol) and Pd/C (2 g) in methanol (300 mL) was stirred at RT under 40 psi of H₂ for 2 h. The reaction mixture was filtered and the filtrate was concentrated to afford ethyl (3-aminophenyl)acetate (17 g). MS (ESI) m/z: 180 (M+H⁺).

To a suspension of (3-aminophenyl)acetic acid (17 g, 94 mmol) in concentrated HCl (50 mL) was added dropwise a solution of sodium nitrite (6.8 g, 0.1 mol) in water at 0° C. The mixture was stirred for 1 h, after which a solution of SnCl₂ (45 g, 0.2 mol) in concentrated HCl was added dropwise at such a rate that the reaction mixture never rose above 5° C. The resulted mixture was stirred for 2 h. The precipitate was collected by suction, washed with ethyl ether to afford ethyl (3-hydrazinophenyl)acetate (15 g). MS (ESI) m/z: 195 (M+H⁺)

A solution of ethyl (3-hydrazinophenyl)acetate (15 g, 65 mmol) and 4,4-dimethyl-3-oxopentanenitrile (12.5 g, 0.1 mol) in EtOH (100 mL) containing concentrated HCl (25 mL) was heated to reflux overnight. After removal of the solvent, the residue was washed with Et₂O to afford ethyl 2-(3-(5-amino-3-t-butyl-1H-pyrazol-1-yl)phenyl)acetate (18 g). MS (ESI) m/z: 302 (M+H⁺).

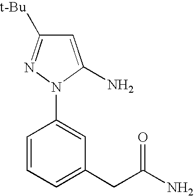

### Example AAA

To a solution of Example YY (6.0 g, 20 mmol) and formamide (1.8 g, 40 mmol) in DMF (20 mL) was added NaOMe (2.1 g, 40 mmol) at RT. The mixture was heated to reflux for 1 h, concentrated and the residue was purified via column chromatography to afford 2-[3-(5-amino-3-t-butyl-1H-pyrazol-1-yl)phenyl]acetamide (2.0 g, 40% yield). ¹H NMR (300 MHz, DMSO-d₆): δ 7.44-7.31 (m, 4H), 7.11 (m, 1H>, 6.87 (br s, 1H), 5.33 (s, 1H), 5.12 (s, 2H), 3.38 (s, 2H), 1.17 (s, 9H); MS (ESI) m/z: 273 (M+H⁺).

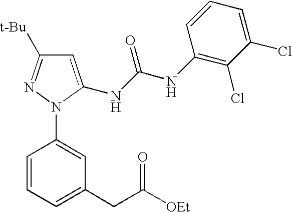

### Example 248

Using the same procedure as for Example 199, Example ZZ (2.0 g, 6.6 mmol) and 1,2-dichloro-3-isocyanato-benzene (1.1 g, 7.5 mmol) were combined to afford 2.2 g of ethyl 2-(3-(3-t-butyl-5-(3-(2,3-dichlorophenyl)ureido)-1H-pyrazol-1-yl)phenyl)acetate. ¹H NMR (300 MHz, DMSO-d₆): 9.22 (s, 1H), 8.75 (s, 1H), 8.05 (m, 1H), 7.46-7.21 (m, 6H), 6.35 (s, 1H), 4.04 (q, J=7.2 Hz, 2 H), 3.72 (s, 2H), 1.24 (s, 9H), 1.16 (t, J=7.2 Hz, 3H); MS (ESI) m/z: 489 (M+H).

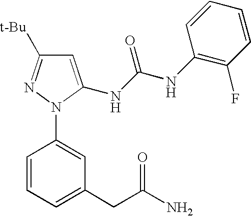

### Example 249

Using the same procedure as for Example 199, Example AAA (136 mg, 0.5 mmol) and 1-fluoro-2-isocyanatobenzene (68 mg, 0.5 mmol) were combined to afford 55 mg of 1-{1-[3-(2-amino-2-oxoethyl)phenyl]-3-t-butyl-1H-pyrazol-5-yl}-3-(2-fluorophenyl)urea (55 mg, 27% yield). ¹H NMR (300 MHz, DMSO-d₆): δ 8.90 (br s, 1H), 8.85 (br s, 1H), 8.05 (br s, 1H), 7.50-7.20 (m, 5H), 7.20-7.00 (m, 2H), 7.00-6.80 (m, 2H), 6.34 (s, 1H), 3.41 (s, 2H), 1.22 (s, 9H).

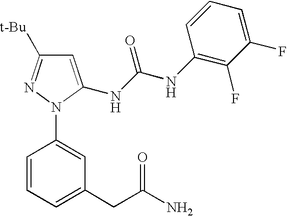

### Example 250

Using the same procedure as for Example 199, Example AAA (136 mg, 0.5 mmol) and 2,3-difluoroaniline (65 mg, 0.5 mmol) were combined to afford 1-{1-[3-(2-amino-2-oxoethyl)phenyl]-3-t-butyl-1H-pyrazol-5-yl}-3-(2,3-difluorophenyl)urea (60 mg, 28% yield). ¹H NMR (300 MHz, CD₃OD-d₄): δ 7.86 (m, 1H), 7.55-7.37 (m, 4H), 7.08 (m, 1H), 6.89 (m, 1H), 6.46 (s, 1H), 3.63 (s, 2H), 1.32 (s, 9H); MS (ESI) m/z: 428 (M+H⁺).

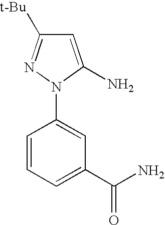

### Example BBB

To a solution of m-aminobenzoic acid (200.0 g, 1.46 mmol) in concentrated HCl (200 mL) was added an aqueous solution (250 mL) of NaNO₂ (102 g, 1.46 mmol) at 0° C. and the reaction mixture was stirred for 1 h. A solution of SnCl₂.2H₂O (662 g, 2.92 mmol) in concentrated HCl (2000 mL) was then added at 0° C. The reaction solution was stirred for an additional 2 h at RT. The precipitate was filtered and washed with ethanol and ether to give 3-hydrazino-benzoic acid hydrochloride as a white solid, which was used for the next reaction without further purification. ¹H NMR (DMSO-d₆): 10.85 (s, 3H), 8.46 (s, 1H), 7.53 (s, 1H), 7.48 (d, J=7.6 Hz, 1H), 7.37 (m, J=7.6 Hz, 1H), 7.21 (d, J=7.6 Hz, 1H).

A mixture of 3-hydrazino-benzoic acid hydrochloride (200 g, 1.06 mol) and 4,4-dimethyl-3-oxo-pentanenitrile (146 g, 1.167 mol) in ethanol (2 L) was heated to reflux overnight. The reaction solution was evaporated under reduced pressure. The residue was purified by column chromatography to give 3-(5-amino-3-t-butyl-pyrazol-1-yl)-benzoic acid ethyl ester (116 g, 40%) as a white solid together with 3-(5-amino-3-t-butyl-pyrazol-1-yl)-benzoic acid (93 g, 36%). 3-(5-amino-3-t-butyl-pyrazol-1-yl)-benzoic acid and ethyl ester: ¹H NMR (DMSO-d₆): 8.09 (s, 1H), 8.05 (brd, J=8.0 Hz, 1H), 7.87 (br d, J=8.0 Hz, 1H), 7.71 (t, J=8.0 Hz, 1H), 5.64 (s, 1H), 4.35 (q, J=7.2 Hz, 2H), 1.34 (t, J=7.2 Hz, 3H), 1.28 (s, 9H).

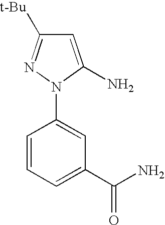

### Example CCC

To a solution of Example T (14.4 g, 50 mmol) and formamide (4.5 g, 0.1 mol) in DMF (50 mL) was added NaOMe (5.4 g 0.1 mol) at RT. The mixture was stirred at 100° C. for 1 h, concentrated and the residue purified by column chromatography to afford 3-(5-amino-3-t-butyl-1H-pyrazol-1-yl)benzamide (6 g, 48% yield).

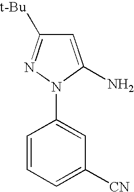

### Example DDD

A solution of Example CCC (5.2 g, 20 mmol) in SOCl₂ (50 mL) was heated to reflux for 6 h. After removal of the solvent, the residue was dissolved in EtOAc (100 mL). The organic layer was washed with saturated NaHCO₃ and brine, dried (Na₂SO₄), filtered, and purified by column chromatography to afford 3-(5-amino-3-t-butyl-1H-pyrazol-1-yl)benzonitrile (3.5 g, 73% yield).

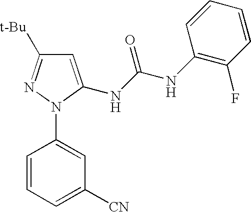

### Example 251

Using the same procedure as for Example 201, Example DDD (120 mg, 0.5 mmol) and 1-fluoro-2-isocynate-benzene (68 mg, 0.5 mmol) were combined to afford 1-[3-t-butyl-1-(3-cyanophenyl)-1H-pyrazol-5-yl]-3-(2-fluorophenyl)urea (55 mg, 29% yield). ¹H NMR (300 MHz, DMSO-d₆): δ 8.90 (br s, 2H), 8.04-7.99 (m, 2H), 7.85 (t, J=8.1 Hz, 2H), 7.70 (t, J=8.1 Hz, 2H), 7.20 (m, 1H), 7.09 (m, 1H), 6.99 (m, 1H), 6.40 (s, 1H), 1.25 (s, 9H); MS (ESI) m/z: 378 (M+H).

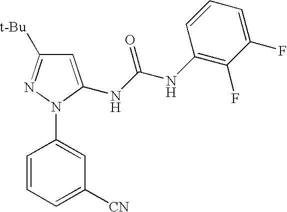

### Example 252

Using the same procedure as for Example 202, Example DDD (120 mg, 0.5 mmol) and 2,3-difluoro-phenylamine (129 mg, 1.0 mmol) were combined to afford 1-[3-t-butyl-1-(3-cyan-phenyl)-1H-pyrazol-5-yl]-3-(2,3-difluorophenyl)urea (55 mg, 28% yield). ¹H NMR (300 MHz, DMSO-d₆): δ 9.07 (br s, 1H), 8.92 (s, 1H), 8.00 (s, 1H), 7.88-7.81 (m, 3H), 7.73 (t, J=7.8 Hz, 1H), 7.12-6.97 (m, 2H), 6.40 (s, 1H), 1.25 (s, 9H); MS (ESI) m/z: 396 (M+H⁺).

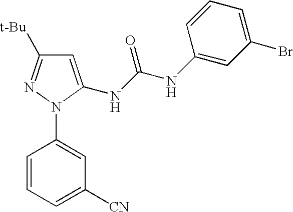

### Example 253

To a stirring suspension of Example DDD (0.0500 g, 0.208 mmol, 1.00 eq) in dry THF (2.0 ml) was added pyridine (0.168 ml, 2.08 mmol, 10.00 eq). The resulting slurry was stirred at RT for 1 h, treated with 3-bromophenyl isocyanate (0.0520 ml, 0.416 mmol, 2.00 eq) and stirred overnight at RT. The reaction was diluted with EtOAc and 1M HCl (10 ml) and the layers separated. The aqueous was extracted with EtOAc (2×), and the combined organic extracts were washed with H₂O (1×), satd. NaHCO3 (1×) and brine (2×), dried (MgSO4), filtered, concentrated, and purified via column chromatography to yield 1-(3-t-butyl-1-(3-cyanophenyl)-1H-pyrazol-5-yl)-3-(3-bromophenyl)urea as an oil (38.4 mg, 42% yield). ¹H NMR (CDCl₃): δ 7.77-7.70 (m, 3H), 7.52 (s, 1H), 7.46-7.44 (m, 2H), 7.35 (s, 1H), 7.16-7.13 (m, 1H), 7.06-7.04 (m, 2H), 6.35 (s, 1H), 1.29 (s, 9H); MS (ESI) m/z: 438.0 (M+H⁺), 440.0 (M+2+H⁺).

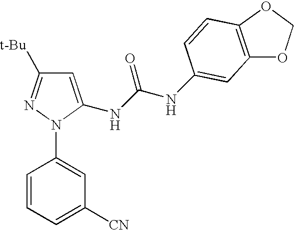

### Example 254

Using the same procedure as for Example 253, Example DDD (0.500 g, 1.81 mmol, 1.00 eq) and 3,4-(methylenedioxy)phenyl isocyanate (0.59 g, 3.62 mmol) were combined to afford 1-(benzo[d][1,3]dioxol-5-yl)-3-(3-t-butyl-1-(3-cyanophenyl)-1H-pyrazol-5-yl)urea as an off-white solid (107.4 mg, 15% yield). ¹H NMR (DMSO-d₆): δ 8.92 (s, 1H), 8.47 (s, 1H), 8.02-8.01 (m, 1H), 7.91-7.89 (m, 1H), 7.86-7.84 (m, 1H), 7.75-7.71 (m, 1H), 7.12-7.11 (m, 1H), 6.82-6.79 (m, 1H), 6.73-6.70 (m, 1H), 6.39 (s, 1H), 5.96 (s, 2H), 1.28 (s, 9H); MS (ESI) m/z: 404.2 (M+H⁺).

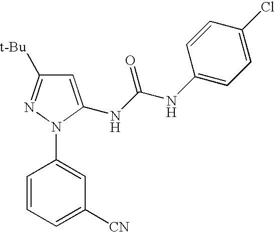

### Example 255

Using the same procedure as for Example 253, Example DDD (0.500 g, 1.81 mmol, 1.00 eq) and 4-chlorophenyl isocyanate (0.555 g, 3.61 mmol) were combined to afford 1-(3-t-butyl-1-(3-cyanophenyl)-1H-pyrazol-5-yl)-3-(4-chlorophenyl)urea (264 mg, 37% yield). ¹H NMR (CDCl₃): δ 7.87 (s, 1H), 7.81-7.79 (m, 1H), 7.58-7.53 (m, 3H), 7.26 (brs, 3H), 6.48 (brs, 1H), 1.37 (s, 9H); MS (ESI) m/z: 394.2 (M+H⁺).

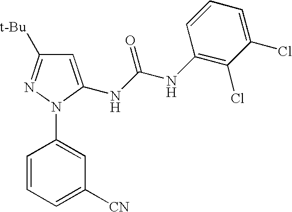

### Example 256

Using the same procedure as for Example 253, Example DDD (0.0500 g, 0.208 mmol) and 2,3-dichlorophenylisocyanate (0.0549 mL, 0.416 mmol) were combined to afford 1-(3-t-butyl)-1-(3-cyanophenyl)-1H-pyrazol-5-yl)-3-(2,3-dichlorophenyl)urea as a white solid (16.9 mg, 19% yield). ¹H NMR (CDCl₃): δ 8.12-8.09 (m, 1H), 7.95 (s, 1H), 7.85-7.83 (m, 1H), 7.64-7.54 (m, 3H), 7.25-7.19 (m, 2H), 6.52 (s, 1H), 1.40 (s, 9H); MS (ESI) m/z: 428.0 (M+H⁺), 430.0 (M+2+H⁺).

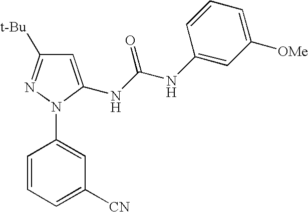

### Example 257

Using the same procedure as for Example 253, Example DDD (0.0500 g, 0.208 mmol) and 3-methoxyphenyl isocyanate (0.0545 mL, 0.416 mmol) were combined to afford 1-(3-t-butyl)-1-(3-cyanophenyl)-1H-pyrazol-5-yl)-3-(3-methoxyphenyl)urea as an oil (15 mg, 19% yield). ¹H NMR (CDCl₃): δ 7.78-7.75 (m, 2H), 7.51-7.44 (m, 2H), 7.33 (s, 1H), 7.24 (s, 1H), 7.18-7.14 (m, 1H), 6.93-6.91 (m, 1H), 6.72-6.70 (m, 1H), 6.65-6.62 (m, 1H), 6.38 (s, 1H), 3.74 (s, 3H), 1.32 (s, 9H); MS (ESI) m/z: 390.2 (M+H⁺).

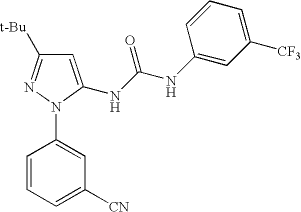

### Example 258

Using the same procedure as for Example 253, Example DDD (0.0500 g, 0.208 mmol) and α,α,α-trifluoro-m-tolyl isocyanate (0.0573 mL, 0.416 mmol) were combined to afford 1-(3-t-butyl)-1-(3-cyanophenyl)-1H-pyrazol-5-yl)-3-(3-(trifluoromethyl)phenyl)urea as an oil (36.7 mg, 41% yield). ¹H NMR (DMSO-d₆): δ 9.40 (s, 1H), 8.64 (s, 1H), 8.05-8.04 (m, 1H), 7.97 (s, 1H), 7.94-7.91 (m, 1H), 7.86-7.84 (m, 1H), 7.75-7.71 (m, 1H), 7.55-7.48 (m, 2H), 7.32-7.31 (m, 1H), 6.44 (s, 1H), 1.30 (s, 9H); MS (ESI) m/z: 428.3 (M+H⁺).

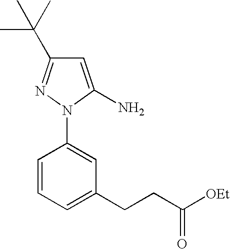

### Example EEE

To a solution of 3-nitro-benzaldehyde (15.1 g, 0.1 mol) in CH₂Cl₂ (200 mL) was added dropwise (triphenyl-15-phosphanylidene)-acetic acid ethyl ester (34.8 g, 0.1 mol) in CH₂Cl₂ (100 mL) at 0° C. After the addition was complete, the resulting mixture was stirred for 1 h. After removal the solvent under reduced pressure, the residue was purified by column chromatography to afford 3-(3-nitro-phenyl)-acrylic acid ethyl ester (16.5 g, 74.6%). ¹H-NMR (400 MHz, CDCl₃): 8.42 (s, 1H), 8.23 (dd, J=0.8 8.0 Hz, 1H), 7.82 (d, J=7.6 Hz, 1H), 7.72 (d, J=16.0 Hz, 1H), 7.58 (t, J=8.0 Hz, 1H), 6.56 (d, J=16.0 Hz, 1H), 4.29 (q, J=7.2 Hz, 2H), 1.36 (t, J=6.8 Hz, 3H).

A mixture of 3-(3-nitro-phenyl)-acrylic acid ethyl ester (16.5 g, 74.6 mmol) and Pd/C (1.65 g) in methanol (200 mL) was stirred under 40 psi of H₂ at RT for 2 h, then filtered through celite. After removal the solvent, 14 g of 3-(3-amino-phenyl)-propionic acid ethyl ester was obtained. ¹H-NMR (400 MHz, CDCl₃): 7.11 (t, J=5.6 Hz, 1H), 6.67 (d, J=7.2 Hz, 1H), 6.63-6.61 (m, 2H), 4.13 (q, J=7.2 Hz, 2H), 2.87 (t, J=8.0 Hz, 2H), 2.59 (t, J=7.6 Hz, 2H), 1.34 (t, J=6.8 Hz, 3H); MS (ESI): m/z: 194 (M+H⁺).

To a solution of 3-(3-amino-phenyl)-propionic acid ethyl ester (14 g, 72.5 mmol) in concentrated HCl (200 mL) was added aqueous (10 mL) of NaNO₂ (5 g, 72.5 mmol) at 0° C. and the resulting mixture was stirred for 1 h. A solution of SnCl₂.2H₂O (33 g, 145 mmol) in concentrated HCl (150 mL) was then added at 0° C. The reaction solution was stirred for an additional 2 h at RT. The precipitate was filtered and washed with ethanol and ether to yield 3-(3-hydrazino-phenyl)-propionic acid ethyl ester as a white solid, which was used for the next reaction without further purification. MS (ESI): m/z: 209 (M+H⁺)

A mixture of 3-(3-hydrazino-phenyl)-propionic acid ethyl ester (13 g, 53.3 mmol) and 4,4-dimethyl-3-oxo-pentanenitrile (6.9 g, 55 mol) in ethanol (150 mL) was heated to reflux overnight. The reaction solution was evaporated under vacuum. The residue was purified by column chromatography to yield ethyl 3-(3-(3-t-butyl-5-amino-1H-pyrazol-1-yl)phenyl)propanoate (14.3 g, 45.4 mmol) as a white solid. ¹H-NMR (300 MHz, DMSO-d₆); 7.50-7.42 (m, 4H), 5.63 (s, 1H), 5.14 (s, 2H), 4.04 (q, J=6.9 Hz, 2H), 2.92 (t, J=7.5 Hz, 2H), 2.66 (t, J=7.5 Hz, 2H), 1.27 (s, 9H), 1.16 (t, J=7.5 Hz, 3H) MS (ESI) m/z: 316 (M+H⁺)

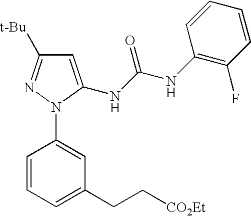

### Example 259

Using the same procedure as for Example 201, Example EEE (101 mg, 1.0 mmol) and 1-fluoro-2-isocyanato-benzene (137 mg, 1.0 mmol) were combined to afford 3-(3-{3-t-butyl-5-[3-(2-fluorophenyl)-ureido]-1H-pyrazol-1-yl}phenyl)propionic acid ethyl ester (240 mg, 53% yield), which was used with further purification.

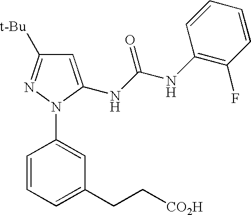

### Example 260

Using the same procedure as for Example 203, Example 256 (100 mg, 0.221 mmol) was saponified to afford 3-(3-{3-t-butyl-5-[3-(2-fluorophenyl)ureido]-1H-pyrazol-1-yl}-phenyl)propionic acid (80 mg, 85% yield). ¹H NMR (300 MHz, DMSO-d₆): δ 8.90 (br s, 1H), 8.81 (s, 1H), 7.08 (t, J=7.5 Hz, 1H), 7.42 (t, J=7.5 Hz, 1H), 7.35 (s, 1H), 7.28 (t, J=6.9 Hz, 1H), 7.28 (m, 1H), 7.07 (t, J=7.5 Hz, 1H), 6.98 (m, 1H), 6.37 (s, 1H), 2.87 (t, J=7.5 Hz, 2H), 2.55 (t, J=7.5 Hz, 2H), 1.24 (s, 9H); MS (ESI) m/z: 425 (M+H⁺).

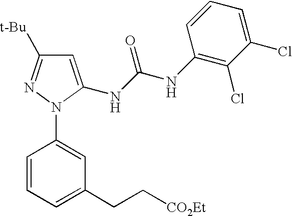

### Example 261

Using the same procedure as for Example 201, Example EEE (300 mg, 1.0 mmol) and 1,2-dichloro-3-isocyanato-benzene (187 mg, 1.0 mmol) were combined to afford 3-(3-{3-t-butyl-5-[3-(2,3-dichlorophenyl)ureido]-1H-pyrazol-1-yl}phenyl)propionic acid ethyl ester (210 mg, 42% yield), which was used without further purification ¹H NMR (DMSO-d₆): δ 9.20 (s, 1H), 8.76 (s, 1H), 8.05 (m, 1H), 7.47-7.26 (m, 6H), 6.38 (s, 1H), 4.04 (q, J=7.2 Hz, 2H), 2.93 (t, J=7.5 Hz, 2H), 2.65 (t, J=7.5 Hz, 2H), 1.28 (s, 9H), 1.15 (t, J=7.2 Hz, 3H); MS (ESI) m/z: 503 (M+H⁺).

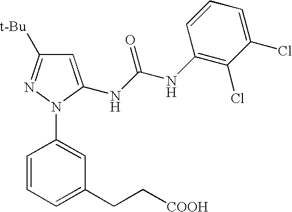

### Example 262

Using the same procedure as for Example 203, Example 262 (100 mg, 0.199 mmol) was saponified to afford 3-(3-{3-t-Butyl-5-[3-(2,3-dichloro-phenyl)ureido]-1H-pyrazol-1-yl}-phenyl)propionic acid (60 mg, 63% yield). ¹H NMR (DMSO-d₆): δ 9.23 (s, 1H), 8.77 (s, 1H), 8.03 (m, 1H), 7.44-7.21 (m, 6H), 6.36 (s, 1H), 2.88 (t, J=7.5 Hz, 2H), 2.58 (t, J=7.5 Hz, 2H), 1.26 (s, 9H); MS (ESI) m/z: 475 (M+H⁺).

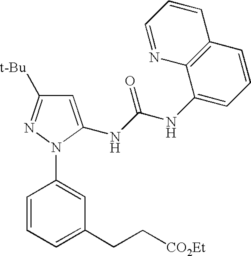

### Example 263

To a solution of Example EEE (150 mg, 0.48 mmol) and NaHCO₃ (200 mg, 2.4 mmol in THF (10 mL) was added a solution of triphosgene (50 mmg, 16 mmol) in THF (1 mL) at 0° C. The mixture was stirred at RT for 1 h, and was subsequently treated with a solution of quinolin-8-ylamine (72 mg, 0.50 mmol) in THF (2 mL). The resulted mixture was stirred for 3 h and concentrated. The residue was dissolved in CH₂Cl₂ (50 mL), and the organic layer was washed with brine, dried (Na₂SO₄), filtered, concentrated and purified by preparative HPLC to afford 3-(3-{3-t-butyl-5-[3-(quinolin-8-yl)ureido]-1H-pyrazol-1-yl}phenyl)-propionic acid ethyl ester (120 mg, 52% yield). ¹H NMR (DMSO-d₆): δ 9.91 (s, 1H), 9.54 (s, 1H), 8.84 (d, J=5.6 Hz, 1H), 8.49 (d, J=6.8 Hz, 1H), 8.35 (d, J=8.0 Hz, 1H), 7.58-7.60 (m, 1H), 7.51-7.53 (m, 2H), 7.36-7.41 (m, 3H), 7.24 (d, J=7.2 Hz, 1H), 6.40 (s, 1H), 3.96 (q, J=7.2 Hz, 1H), 2.88 (t, J=7.6 Hz. 2H), 2.60 (t, J=7.6 Hz, 2H), 1.28 (s, 9H), 1.07 (t, J=7.2 Hz, 3H); MS (ESI) m/z: 486 (M+H⁺).

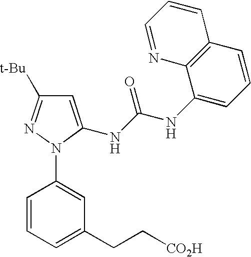

### Example 264

Using the same procedure as for Example 203, Example 263 (70 mg, 0.14 mmol) was saponified to afford 3-(3-{3-t-butyl-5-[3-(quinolin-8-yl)ureido]-1H-pyrazol-1-yl}phenyl)-propionic acid (50 mg, 78% yield). ¹H NMR (DMSO-d₆): δ 9.93 (s, 1H), 9.56 (s, 1H), 8.84 (d, J=4.0 Hz, 1H), 8.50 (d, J=6.8 Hz, 1H), 8.36 (d, J=6.8 Hz, 1H), 7.60 (m, 1H), 7.40-7.50 (m, 4H), 7.34 (d, J=8.4 Hz, 1H), 7.25 (d, J=7.6 Hz, 1H), 6.41 (s, 1H), 2.87 (t, J=7.6 Hz, 2H), 2.55 (t, J=7.6 Hz, 2H), 1.23 (s, 9H); MS (ESI) m/z: 458 (M+H⁺).

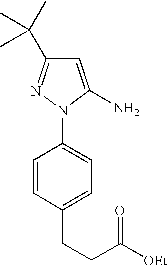

### Example FFF

To a solution of 4-nitro-benzaldehyde (15.1 g, 0.1 mol) in CH₂Cl₂ (200 mL) was added dropwise (triphenyl-15-phosphanylidene)-acetic acid ethyl ester (34.8 g, 0.1 mol) in dichloromethane (100 mL) under 0° C. in ice-bath. After the addition was completed, the resulting mixture was stirred for 2 h. After removal the solvent under reduced pressure, the residue was purified by column chromatography to afford 3-(4-nitrophenyl)-acrylic acid ethyl ester (16.5 g, 74.6%) ¹H-NMR (400 MHz, CDCl₃): 8.25 (d, J=8.8 Hz, 2H), 7.71 (d, J=16.0 Hz, 1H), 7.67 (d, J=8.8 Hz, 2H), 6.55 (d, J=16.0 Hz, 2H), 4.29 (q, J=7.2 Hz, 2H), 1.34 (t, J=7.2 Hz, 3H).

A mixture of 3-(4-nitro-phenyl)-acrylic acid ethyl ester (16.5 g, 74.6 mmol) and Pd/C (1.65 g) in methanol (200 mL) was stirred under 40 psi of H₂ at RT at 2 h before filtered over celite. After removal the solvent, 14 g of 3-(4-amino-phenyl)-propionic acid ethyl ester was obtained. ¹H-NMR (400 MHz, CDCl₃): 6.98 (d, J=8.0 Hz, 2H), 6.61 (d, J=8.4 Hz, 1H), 4.12 (q, J=7.2 Hz, 2H), 2.84 (t, J=8.0 Hz, 2H), 2.55 (t, J=7.6 Hz, 2H), 1.23 (t, J=7.2 Hz, 3H); MS (ESI): m/z: 194 (M+H⁺).

To a solution of 3-(4-amino-phenyl)-propionic acid ethyl ester (14 g, 72.5 mmol) in conc. HCl (200 mL) was added aqueous (10 mL) of NaNO₂ (5 g, 72.5 mmol) at 0° C. and the resulting mixture was stirred for 1 h. A solution of SnCl₂.2H₂O (33 g, 145 mmol) in conc. HCl (150 mL) was then added at 0° C. The reaction solution was stirred for an additional 2 h at RT. The precipitate was filtered and washed with ethanol and ether to yield 3-(4-hydrazino-phenyl)-propionic acid ethyl ester as a white solid, which was used for the next reaction without further purification; MS (ESI): m/z: 209 (M+H⁺).

A mixture of 3-(4-hydrazino-phenyl)-propionic acid ethyl ester (13 g, 53.3 mmol) and 4,4-dimethyl-3-oxo-pentanenitrile (6.9 g, 55 mol) in ethanol (150 mL) was heated to reflux overnight. The reaction solution was evaporated under vacuum. The residue was purified by column chromatography to yield ethyl 3-(4-(3-t-butyl-5-amino-1H-pyrazol-1-yl)phenyl)-propanoate (14.3 g, 45.4 mmol) as a white solid. ¹H-NMR (300 MHz, DMSO-d₆); 7.44 (d, J=8.4 Hz, 2H), 7.27 (d, J=8.7 Hz, 2H), 5.34 (s, 1H), 5.11 (s, 2H), 4.04 (q, J=7.2 Hz, 2H), 2.86 (t, J=7.5 Hz, 2H), 2.61 (t, J=7.5 Hz, 2H), 1.19 (s, 9H), 1.15 (t, J=7.2 Hz, 3H) MS (ESI) m/z: 316 (M+H⁺)

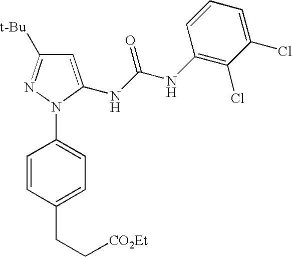

### Example 265

Using the same procedure as for Example 201, Example FFF (300 mg, 1.0 mmol) and 1,2-dichloro-3-isocyanato-benzene (187 mg, 1.0 mmol) were combined to afford 3-(4-{3-t-butyl-5-[3-(2,3-dichlorophenyl)ureido]-1H-pyrazol-1-yl}phenyl)propionic acid ethyl ester (250 mg, 50% yield), which was used without further purification. MS (ESI) m/z: 503 (M+H⁺).

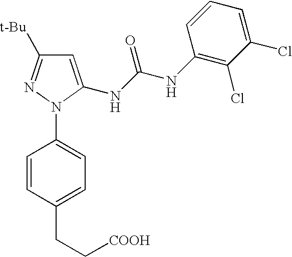

### Example 266

Using the same procedure as for Example 203, Example 265 (100 mg, 0.199 mmol) was saponified to afford 60 mg of 3-(3-{3-t-Butyl-5-[3-(2,3-dichloro-phenyl)-ureido]-pyrazol-1-yl}-phenyl)-propionic acid (60 mg, 64% yield). ¹H NMR (DMSO-d₆): δ 9.29 (s, 1H), 8.80 (s, 1H), 8.04 (m, 1H), 7.44-7.33 (m, 4H), 7.29-7.27 (m, 2H), 6.36 (s, 1H), 2.87 (t, J=7.5 Hz, 2H), 2.57 (t, J=7.5 Hz, 2H), 1.25 (s, 9H); MS (ESI) m/z: 475 (M+H⁺).

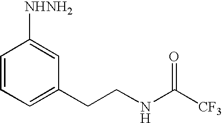

### Example GGG

To a stirring solution of 3-nitrophenylacetic acid (10.4 g, 57.3 mmol) in MeOH (250 ml) at RT was added HCl gas until saturation was achieved. The resulting solution was stirred at 70° C. for 1 h. The reaction was cooled and concentrated under reduced pressure. The semisolid residue was dissolved in Et₂O, washed with H₂O (2×), sat'd. NaHCO₃ (2×), brine (1×) and dried (MgSO₄). Filtration and evaporation provided methyl 2-(3-nitrophenyl)acetate as a low-melting solid (10.7 g, 96% yield), which was used without further purification. ¹H NMR (CDCl₃): δ 8.14-8.04 (m, 2H), 7.64-7.58 (m, 1H), 7.47 (br t, J=8.10 Hz, 1H), 3.72 (s, 2H), 3.68 (s, 3H); MS (ESI) m/z: 196.0 (M+H⁺).

Methyl 2-(3-nitrophenyl)acetate (9.6 g, 49 mmol) was treated with conc. NH₄OH (24 ml, 172 mmol). The suspension was stirred briskly at RT until complete, then chilled thoroughly in an ice bath. The solids were collected by filtration, rinsed sparingly with ice water and dried to yield pure 2-(3-nitrophenyl)acetamide as an off-white solid (7.47 g, 84% yield)). ¹H NMR (DMSO-d₆): δ 8.18-8.02 (m, 2H), 7.75-7.70 (m, 1H), 7.61-7.57 (m, 3H), 7.00 (br s, 1H), 3.58 (s, 3H); MS (ESI) m/z: 181.0 (M+H⁺).

To a stirring solution of borane-THF (3.5 ml, 3.5 mmol, 1.0M) was added a solution of 2-(3-nitrophenyl)acetamide (0.25 g, 1.4 mmol) in THF (7.0 ml) at RT. The resulting solution was stirred at RT until the gas evolution had subsided and then was heated at 70° C. overnight. The cooled reaction was quenched carefully with 3M. HCl (2 ml), then 70° C. to complete the quench. The reaction was cooled to RT and concentrated to a white solid, which was dissolved in 3M NaOH (pH 14) and extracted with CH₂Cl₂ (4×). The organics were dried (Na₂SO₄), filtered, and concentrated to provide 0.20 g (87%) of crude product as an oil, which was purified by precipitation from CH₂Cl₂ and 3M HCl/EtOAc (0.26 ml, 0.78 mmol) to yield 2-(3-nitrophenyl)ethanamine as the HCl salt as an off-white solid (0.164 g). ¹H NMR (DMSO-d₆): δ 8.18-8.15 (m, 1H), 8.13-8.04 (m, 1H), 8.02 (br s, 3H), 7.76-7.74 (m, 1H), 7.65 (br t, J=7.84 Hz), 3.17-3.08 (m, 2H), 3.06-3.00 (m, 2H); MS (ESI) m/z: 167.0 (M+H⁺).

To a stirring suspension of 2-(3-nitrophenyl)ethanamine hydrochloride (0.164 g, 0:81 mmol) in dry CH₂Cl₂ (8 ml) at RT was added DIEA (0.42 ml, 2.43 mmol). The reaction was stirred at RT until the solids were dissolved, then cooled thoroughly in an ice bath and TFAA (0.14 ml, 1.01 mmol) was added dropwise. The resulting yellow solution was stirred overnight with slow warming to RT. The reaction mixture was washed with ice H₂O (2×) and dried (MgSO₄). Filtration and evaporation provided N-(3-nitrophenethyl)-2,2,2-trifluoro-acetamide (0.215 g, 101% yield) of as an oil that solidified on standing. ¹H NMR (CDCl₃): δ 8.17-8.14 (m, 1H), 8.11-8.10 (m, 1H), 7.58-7.52 (m, 2H), 6.4 (brs, 1H), 3.70 (q, J=6.00 Hz, 2H), 3.06 (t, J=6.00 Hz, 2H).

To a solution of N-(3-nitrophenethyl)-2,2,2-trifluoroacetamide (9.05 g, 34.5 mmol) in MeOH (125 ml) at RT was added 10% Pd/C (50% water wet) (3.67 g, 1.73 mmol). The resulting suspension was placed under 3 atm of H₂ at 20-25° C. overnight. The reaction was filtered through Celite and the cake rinsed with MeOH. The filtrate was concentrated to provide N-(3-aminophenethyl)-2,2,2-trifluoroacetamide as an oil (7.83 g, 98% yield). ¹H NMR (CDCl₃): δ 7.16-7.12 (m, 1H), 6.62-6.58 (m, 2H), 6.54-6.53 (m, 1H), 6.34 (brs, 1H), 3.61 (q, J=6.40 Hz, 2H), 2.80 (t, J=6.40 Hz, 2H), 2.68 (brs, 2H); MS (ESI) m/z: 233.3 (M+H⁺).

To a stirring solution of N-(3-aminophenethyl)-2,2,2-trifluoroacetamide (7.83 g, 33.7 mmol) in EtOAc (80 ml) at RT was added 3M HCl/EtOAc (12.4 ml, 37.1 mmol). Solids precipitated almost immediately. The resulting suspension was cooled in ice 1 h. The solids were collected by filtration, rinsed with EtOAc and dried on the filter. There was obtained pure N-(3-aminophenethyl)-2,2,2-trifluoroacetamide hydrochloride free of less polar impurities as a pale tan solid (7.94 g, 88% yield). ¹H NMR (DMSO-d₆): δ 10.36 (br s, 3H), 9.61 (t, J=5.32 Hz, 1H), 7.43-7.39 (m, 1H), 7.25-7.23 (m, 2H), 3.42 (q, J=6.60 Hz, 2H), 2.84 (t, J=6.60 Hz, 2H).

N-(3-aminophenethyl)-2,2,2-trifluoroacetamide hydrochloride (0.27 g, 1.0 mmol) was suspended in 6M HCl (2.0 ml) and cooled thoroughly in an ice bath. This was rapidly stirred while a solution of sodium nitrite (73 mg) in H₂O (1.0 ml) was added slowly. The mixture was stirred at 0-5° C. for 45 min and was then treated with tin chloride dihydrate (1.3 g, 5.8 mmol) in 6M HCl (4.0 ml). The resulting suspension was stirred at 0-5° C. for 3 h and then carefully quenched with 3M NaOH (15 mL) to pH 7-8. The mixture was diluted with Et₂O, filtered through Celite and the filter cake was washed with H₂O and with Et₂O. The layers of the biphasic filtrate were separated and the aqueous extracted with Et₂O (2×). The combined organics extracts were washed with brine (1×), dried (Na₂SO₄), filtered and evaporated to provided N-(3-hydrazinophenethyl)-2,2,2-trifluoroacetamide as a pale yellow oil (0.18 g, 72% yield), which was used without further purification. MS (ESI) m/z: 248.0 (M+H⁺).

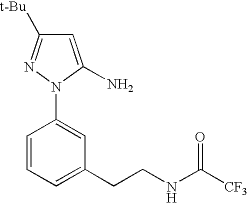

### Example HHH

To a stirring solution of Example GGG (0.18 g, 0.73 mmol) in absolute EtOH (5 ml) at RT was added pivaloylacetonitrile (0.11 g, 0.87 mmol) and sat'd. HCl/EtOH (3 drops from a pipet). The resulting solution was stirred at 75-80° C. overnight, then cooled to RT and concentrated. The residue was dissolved in Et₂O and washed with sat'd. NaHCO₃. The aqueous was extracted with Et₂O (1×). The combined organics were washed with brine (1×) and dried (MgSO₄), filtered, concentrated and purified via flash chromatography to provide N-[3-(5-amino-3-t-butyl-1H-pyrazol-1-yl)phenethyl]-2,2,2-trifluoroacetamide as an orange glass (0.18 g, 70% yield). ¹H NMR (CDCl₃): δ 7.47-7.46 (m, 2H), 7.43-7.39 (m, 1H), 7.14-7.12 (m, 1H), 5.51 (s, 1H), 3.67 (q, J=6.48 Hz, 2H), 2.95 (t, J=6.48 Hz, 2H), 1.33 (s, 9H); MS (ESI) m/z: 355.2 (M+H⁺).

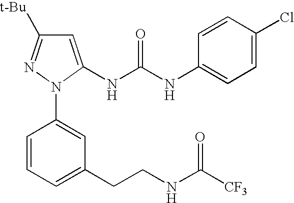

### Example 267

To a stirring solution of Example HHH (0.180 g, 0.51 mmol) in dry CH₂Cl₂ (5 ml) at RT was added 4-chlorophenyl isocyanate (82 mg, 0.53 mmol). The resulting mixture was stirred at RT overnight. More 4-chlorophenyl isocyanate was added (40 mg, 0.26 mmol) and stirring was continued. After 2 h, the reaction was concentrated to dryness and purified by flash chromatography to yield pure 1-(3-t-butyl-1-{3-[2-(2,2,2-trifluoroacetamido)ethyl]-phenyl})-1H-pyrazol-5-yl)-3-(4-chlorophenyl)urea as an orange foam (0.134 g, 52% yield). ¹H NMR (CDCl₃): δ 8.14 (br s, 1H), 7.39-7.20 (m, 8H), 7.03 (br s, 1H), 6.57 (s, 1H), 3.77 (m, 2H), 2.88 (m, 2H), 1.35 (s, 9H); MS (ESI) m/z: 508.3 (M+H⁺).

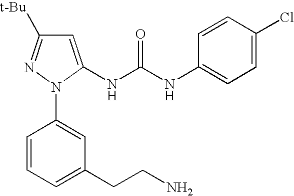

### Example 268

To a stirring solution of Example 267 (0.134 g, 0.264 mmol) in MeOH (10 ml) and H₂O (0.6 ml) at RT was added potassium carbonate (0.182 g, 1.32 mmol). The resulting suspension was stirred at 60-65° C. for 2 h, then cooled to RT and the volatiles evaporated. The residue was carefully dissolved in 1M HCl to pH I-2 and extracted with Et₂O (2×). The aqueous was then basified (pH 13-14) with 3M NaOH and extracted with CH₂Cl₂ (4×). The combined CH₂Cl₂ extracts were washed with brine (1×), dried (Na₂SO₄), filtered, and concentrated to provided 1-{1-[3-(2-aminoethyl)phenyl]-3-t-butyl-1H-pyrazol-5-yl}-3-(4-chlorophenyl)urea as a foam (70.6 mg, 65% yield). ¹H NMR (CDCl₃): δ 8.64 (br s, 1H), 7.33-7.00 (m, 8H), 6.39 (s, 1H), 2.65 (m, 4H), 1.31 (s, 9H); MS (ESI) m/z: 412.3 (M+H⁺).

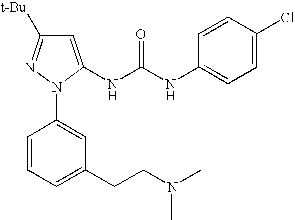

### Example 269

To a stirring solution of Example 268 (50 mg, 0.12 mmol) in MeOH (1.2 ml) at RT was added aq. formaldehyde (37 wt %, 0.036 ml, 0.49 mmol) and conc. formic acid (0.037 ml, 0.97 mmol). The reaction was stirred at 60-65° C. overnight, then cooled to RT, diluted with 1M HCl and filtered. The filtrate was made basic (pH 13) with 3M NaOH and extracted with CH₂Cl₂ (2×). The combined organics were washed with brine (1×), dried (Na₂SO₄), filtered, concentrated and purified by column chromatography, to yield 1-{3-t-butyl-1-[3-(2-(dimethylamino)ethyl]phenyl}-1H-pyrazol-5-yl)-3-(4-chlorophenyl)urea (12.5 mg, 23% yield) of product as a glass. ¹H NMR (CDCl₃): δ 8.33 (br s, 1H), 8.26 (br s, 1H), 7.43-7.06 (m, 8H), 6.51 (s, 1H), 2.84 (t, J=6.3 Hz, 2H), 2.75 (t, J=6.3 Hz, 2H), 2.27 (s, 6H), 1.36 (s, 9H); MS (ESI) m/z: 440.2 (M+H⁺).

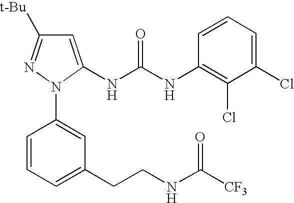

### Example 270

To a stirring solution of Example HHH (50 mg, 0.14 mmol) in dry THF (1.0 ml) at RT was added pyridine (0.11 ml, 1.4 mmol) followed by 2,3-dichlorophenyl isocyanate (0.037 ml, 0.28 mmol). The reaction was stirred overnight at RT, then diluted with 1M HCl (10 ml) and stirred for 1 h. The mixture was extracted with EtOAc (3×). The combined organic extracts were washed with H₂O (1×), satd. NaHCO₃ (1×), brine (1×), then dried (MgSO₄) filtered, concentrated and purified via column chromatography to provide 1-{3-t-butyl-1-(3-[2-(2,2,2-trifluoroacetamido)ethyl]phenyl}-1H-pyrazol-5-yl)-3-(2,3-dichlorophenyl)urea (32.2 mg, 42% yield). ¹H NMR (CDCl₃): δ 8.19 (dd, J=1.92, 7.92 Hz, 1H), 8.02 (br s, 1H), 7.88 (br s, 1H), 7.45-7.36 (m, 3H), 7.22-7.15 (m, 3H), 7.05 (br d, J=7.44 Hz, 1H), 6.59 (s, 1H), 3.78 (q, J=6.44 Hz, 2H), 2.90 (t, J=6.4 Hz, 2H), 1.37 (s, 9H); (ESI) m/z: 542.3 (100, M+H⁺), 543.2 (30, M+2), 544.2 (66, M+3).

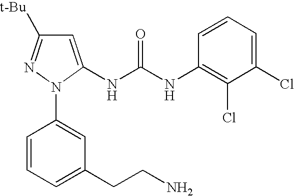

### Example 271

To a stirring solution of Example 270 (32.2 mg, 0.059 mmol) in MeOH (1.80 ml) and H₂O (0.15 ml) at RT was added potassium carbonate (41.0 g, 0.297 mmol). The resulting suspension was stirred at 60-65° C. for 2 h. The reaction was cooled to RT, diluted with H₂O and extracted with CHCl₃ (3×). The combined organics were washed with brine (1×), dried (Na₂SO₄), filtered and concentrated to provide 1-{1-[3-(2-aminoethyl)phenyl]-3-t-butyl-1H-pyrazol-5-yl}-3-(2,3-dichlorophenyl)urea as a waxy solid (25.6 mg, 97% yield). ¹H NMR (CDCl₃): δ 8.17 (dd, J=1.24, 8.08 Hz, 1H), 7.31-7.28 (m, 4H), 7.14-7.06 (m, 4H), 6.45 (s, 1H), 3.48 (br t, J=4.4 Hz, 2H), −3.46-3.39 (m, 2H), 2.86 (t, J=7.0 Hz, 2H), 1.3 (s, 9H).

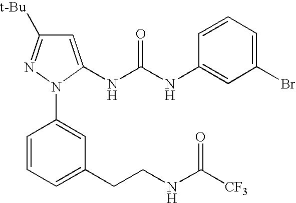

### Example 272

Using the same procedure as for Example 270, Example HHH (50 mg, 0.14 mmol) and 3-bromophenyl isocyanate (0.035 ml, 0.28 mmol) were combined to afford 1-(3-bromophenyl)-3-(3-t-butyl-1-{3-[2-(2,2,2-trifluoroacetamido)ethyl]phenyl}-1H-pyrazol-5-yl)urea (20.6 mg, 26% yield). ¹H NMR (CDCl₃): δ 8.17 (s, 1H), 7.66 (t, J=1.76 Hz, 1H), 7.49 (t, J=6.48 Hz, 1H), 7.42 (s, 1H), 7.37-7.34 (m, 3H), 7.23-7.20 (1H), 7.17-7.05 (m, 3H), 6.58 (s, 1H), 3.78 (q, J=6.4 Hz, 2H), 2.89 (t, J=6.1 Hz, 2H), 1.36 (s, 9H); MS (ESI) m/z: 552.2 (100, M+H⁺), 554.2 (98, M+2).

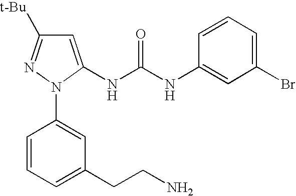

### Example 273

Using the same procedure as for Example 217, Example 272 (20.6 mg, 0.037 mmol) was deprotected to provide 1-{1-[3-(2-aminoethyl)phenyl]-3-t-butyl-1H-pyrazol-5-yl}-3-(3-bromophenyl)urea (22.4 mg). ¹H NMR (CDCl₃): δ 8.30 (br s, 1H), 7.53 (br s, 1H), 7.32-7.0 (m, 8H), 6.41 (s, 1H), 3.0-2.7 (br s, 4H), 1.34 (s, 9H); MS (ESI) m/z: 456.2 (100, M+H), 458.2 (98, M+2).

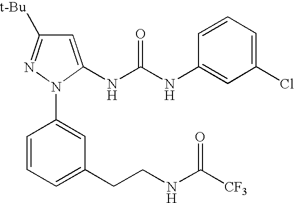

### Example 274

Using the same procedure as for Example 270, Example HHH (50 mg, 0.14 mmol) and 3-chlorophenyl isocyanate (0.034 ml, 0.28 mmol) were combined to afford 1-(3-t-butyl-1-{3-[2-(2,2,2-trifluoroacetamido)ethyl]phenyl}-1H-pyrazol-5-yl)-3-(3-chlorophenyl)urea (32.2 mg, 45% yield). ¹H NMR (CDCl₃): δ 8.18 (s, 1H), 7.51-7.48 (m, 2H), 7.43 (s, 1H), 7.37-7.34 (m, 3H), 7.20-7.14 (m, 2H), 7.08-7.05 (m, 1H), 7.02-6.99 (m, 1H), 6.58 (s, 1H), 3.78 (q, J=6.4 Hz, 2H), 2.88 (t, J=6.4 Hz, 2H), 1.36 (s, 9H); MS (ESI) m/z: 508.3 (100, M+H⁺), 510.2 (37, M+2).

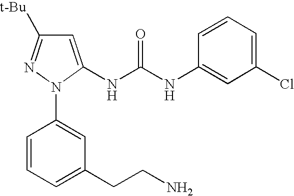

### Example 275

Using the same procedure as for Example 271, Example 274 (32.2 mg, 0.063 mmol) was deprotected to afford 1-{1-[3-(2-aminoethyl)phenyl]-3-t-butyl-1H-pyrazol-5-yl}-3-(3-chlorophenyl)urea (19.1 mg, 73% yield). ¹H NMR (CDCl₃): δ 8.29 (br s, 1H), 7.46 (s, 1H), 7.43-7.29 (m, 1H), 7.23-7.19 (m, 2H), 7.16-7.10 (m, 3H), 7.01-6.97 (m, 2H), 6.41 (s, 1H), 2.94 (br s, 2H), 2.71 (br s, 2H), 1.34 (s, 9H); MS (ESI) m/z: 412.3 (100, M+H⁺), 414.2 (36, M+2).

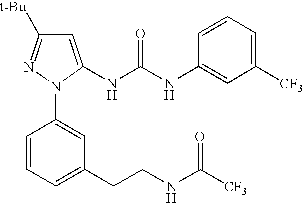

### Example 276

Using the same procedure as for Example 270, Example HHH (50 mg, 0.14 mmol) and α,α,α-trifluoro-m-tolyl isocyanate (0.039 ml, 0.28 mmol) were combined to provide 1-(3-t-butyl-1-{3-[2-(2,2,2-trifluoroacetamido)ethyl]phenyl}-1H-pyrazol-5-yl)-3-[3-(trifluoromethyl)phenyl]urea (31.1 mg, 41% yield). ¹H NMR (CDCl₃): δ 8.23 (s, 1H), 7.66 (s, 1H), 7.61-7.59 (m, 1H), 7.42-7.36 (m, 4H), 7.27-7.26 (m, 1H), 7.12-7.09 (m, 1H), 7.08-7.05 (m, 1H), 6.64 (s, 1H), 3.88 (q, J=5.5 Hz, 2H), 2.95 (t, J=5.5 Hz, 2H), 1.37 (s, 9H); MS (ESI) m/z: 542.3 (M+H⁺).

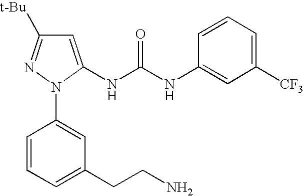

### Example 277

Using the same procedure as for Example 271, Example 276 (31.1 mg, 0.057 mmol) was deprotected to provide 1-(1-[3-(2-aminoethyl)phenyl]-3-t-butyl-1H-pyrazol-5-yl)-3-[3-(trifluoromethyl)phenyl]urea (24.8 mg, 97% yield). ¹H NMR (CDCl₃): δ 8.34 (brs, 1H), 7.60 (s, 1H), 7.54-7.50 (m, 1H), 7.37-7.37.25 (m, 5H), 7.18-7.17 (m, 1H), 6.44 (s, 1H), 2.99 (br s, 2H), 2.75 (br s, 2H), 1.35 (s, 9H); MS (ESI) m/z: 446.3 (M+H⁺).

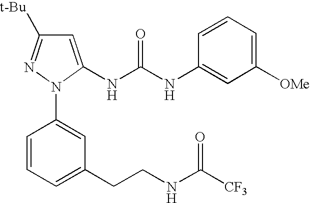

### Example 278

Using the same procedure as for Example 270, Example HHH (50 mg, 0.14 mmol) and 3-methoxyphenyl isocyanate (0.037 ml, 0.28 mmol) were combined to afford 1-(3-t-butyl-1-{3-[2-(2,2,2-trifluoroacetamido)ethyl]phenyl}-1H-pyrazol-5-yl)-3-(3-methoxyphenyl)urea (29.6 mg, 42% yield). ¹H NMR (CDCl₃): δ 8.01 (s, 1H), 7.39-7.34 (m, 4H), 7.28-7.24 (m, 1H), 7.18-7.14 (m, 1H), 7.11-7.09 (m, 1H), 7.08-7.06 (m, 1H), 7.06-7.05 (m, 11H), 6.87-6.84 (m, 1H), 6.61-6.60 (m, 1H), 6.59 (s, 1H), 3.78 (q, J=6.6 Hz, 2H), 3.77 (s, 3H), 2.88 (t, J=6.6 Hz, 21), 1.36 (s, 9H); MS (ESI) m/z: 504.2 (M+H⁺).

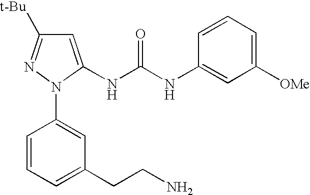

### Example 279

Using the same procedure as for Example 271, Example 278 (29.6 mg, 0.059 mmol) was deprotected to provide 1-{1-[3-(2-aminoethyl)phenyl]-3-t-butyl-1H-pyrazol-5-yl}-3-(3-methoxyphenyl)urea (16.4 mg, 69% yield). ¹H NMR (CDCl₃): δ 7.89 (br s, 1H), 7.34-7.27 (m, 4H), 7.16-7.13 (m, 2H), 7.06 (br s, 1H), 6.78-6.76 (m, 1H), 6.61-6.58 (m, 1H), 6.41 (s, 1H), 3.76 (s, 3H), 2.96 (br s, 2H), 2.75 (br s, 2H), 1.35 (s, 9H); MS (ESI) m/z: 408.3 (M+H⁺).

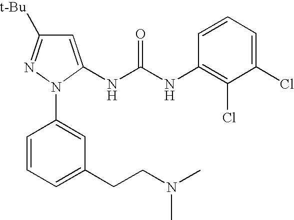

### Example 280

Using the same procedure as for Example 269, Example 271 (54.2 mg, 0.121 mmol) was obtained 1-(3-t-butyl-1-{3-[2-(dimethylamino)ethyl]phenyl}-1H-pyrazol-5-yl)-3-(2,3-di-chlorophenyl)urea (17.4 mg, 30% yield).

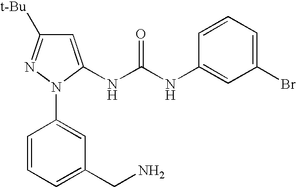

### Example 281

To a stirring solution of Example 253 (0.17 g, 0.39 mmol) in dry THF (4 ml) at RT was added 1.0M LAH in THF (0.58 ml, 0.58 mmol). After 2 h at RT additional 11.0M LiAlH4 in THF (0.58 ml, 0.58 mmol) was added and the reaction was stirred an 1 h. The reaction was carefully quenched by the addition of H₂O (0.044 ml), 3M NaOH (0.044 ml) and H₂O (0.088 ml) and stirred overnight at RT. The mixture was filtered through Celite, rinsing generously with EtOAc. The filtrate was concentrated to dryness to give 0.13 g of crude product, which was redissolved in EtOAc and treated with 3M HCl/EtOAc. A precipitate formed immediately, which was collected by filtration, rinsed with EtOAc and dried to yield 1-{1-[3-(aminomethyl)phenyl]-3-t-butyl-1H-pyrazol-5-yl}-3-(3-bromophenyl)urea as the HCl salt (0.131 g, 70% yield). ¹H NMR (DMSO-d₆): δ 9.93 (s, 1H), 8.83 (s, 1H), 8.36 (br s, 3H), 7.82-7.81 (m, 1H), 7.71 (br s, 1H), 7.57-7.55 (m, 2H), 7.48-7.46 (m, 1H), 7.31-7.29 (m, 1H), 7.24-7.20 (m, 1H), 7.15-7.13 (m, 1H), 6.42 (s, 1H), 4.16-4.12 (m, 2H), 1.29 (s, 9H); MS (ESI) m/z: 442.3 (M+H⁺), 444.2 (M+2+W).

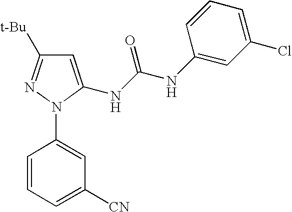

### Example 282

Using the same procedure as for Example 201, Example DDD (0.0500 g, 0.208 mmol) and 3-chlorophenyl isocyanate (0.0507 mL, 0.416 mmol) were combined to afford 1-(3-t-butyl-1-(3-cyanophenyl)-1H-pyrazol-5-yl)-3-(3-chlorophenyl)urea as an oil (32.8 mg, 40% yield)). ¹H NMR (CDCl₃): δ 7.79-7.76 (m, 2H), 7.60 (s, 1H), 7.48-7.44 (3H), 7.26-7.25 (m, 1H), 7.16-7.12 (m, 1H), 7.05-7.01 (m, 2H), 6.37 (s, 1H), 1.31 (s, 9H); MS (ESI) m/z: 394.2 (M+H⁺), 396.3 (M+2+H⁺).

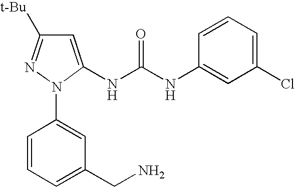

### Example 283

Using the same procedure as for Example 281, Example 112 (0.11 g, 0.28 mmol) was reduced to afford 1-{1-[3-(aminomethyl)phenyl]-3-t-butyl-1H-pyrazol-5-yl}-3-(3-chloro-phenyl)urea as an off-white HCl salt (77.2 mg, 64% yield). ¹H NMR (DMSO-d₆): δ 10.11 (s, 1H), 8.91 (s, 1H), 8.43 (br s, 3H), 7.72 (s, 1H), 7.68 (s, 1H), 7.56-7.55 (m, 2H), 7.48-7.46 (m, 1H), 7.31-7.25 (m, 2H), 7.02-6.99 (m, 1H), 6.42 (s, 1H), 4.16-4.12 (m, 2H), 1.30 (s, 9H); MS (ESI) m/z: 398.3 (M+H⁺), 400.2 (M+2+H⁺).

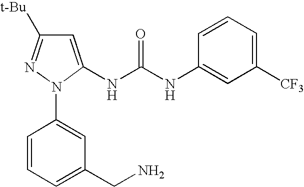

### Example 284

Using the same procedure as for Example 281, Example 258 (0.120 g, 0.28 mmol) was reduced to afford 1-{1-[3-(aminomethyl)phenyl]-3-t-butyl-1H-pyrazol-5-yl}-3-(3-(trifluoro-methyl)phenyl)urea as an off-white HCl salt (73.9 mg, 56% yield). ¹H NMR (DMSO-d₆): δ 10.26 (s, 1H), 8.94 (s, 1H), 8.42 (br s, 3H), 7.98 (s, 1H), 7.73 (s, 1H), 7.58-7.47 (m, 5H), 7.32-7.30 (m, 1H), 6.44 (s, 1H), 4.14 (m, 2H), 1.29 (s, 9H); MS (ESI) m/z: 432.2 (M+H⁺)

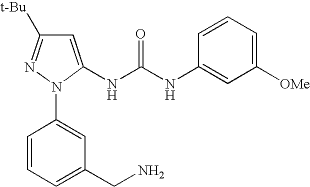

### Example 285

Using the same procedure as for Example 281, Example 257 (0.16 g, 0.411 mmol) was reduced to afford 1-{1-[3-(aminomethyl)phenyl]-3-t-butyl-1H-pyrazol-5-yl}-3-(3-methoxy-phenyl)urea as an off-white HCl salt (137 mg, 77% yield). ¹H NMR (DMSO-d₆): δ 9.75 (s, 1H), 8.80 (s, 1H), 8.43 (br s, 3H), 7.72 (s, 1H), 7.56-7.55 (m, 2H), 7.49-7.47 (m, 1H), 7.18-7.13 (m, 2H), 6.92-6.89 (m, 1H), 6.55-6.53 (m, 1H), 6.41 (s, 1H), 4.16-4.12 (m, 2H), 3.71 (s, 3H), 1.29 (s, 9H); MS (ESI) m/z: 394.2 (M+H⁺).

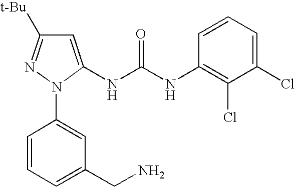

### Example 286

Using the same procedure as for Example 281, Example 256 (50 mg, 0.12 mmol) was reduced to afford 1-{1-[3-(aminomethyl)phenyl]-3-t-butyl-1H-pyrazol-5-yl}-3-(2,3-dichloro-phenyl)urea as a white solid (20.6 mg, 41% yield). ¹H NMR (CDCl₃): δ 9.55 (s, 1H), 8.47 (br s, 3H), 7.97-7.96 (m, 1H), 7.70-7.32 (m, 4H), 7.15-7.11 (m, 3H), 6.81 (s, 1H), 4.10 (br s, 2H), 1.38 (s, 9H); MS (ESI) m/z: 432.2 (M+H⁺), 434.2 (M+2+H).

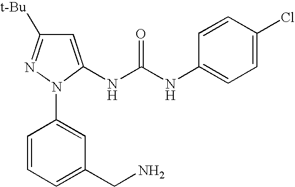

### Example 287

Using the same procedure as for Example 281, Example 255 (87 mg, 0.22 mmol) was reduced to afford 1-{1-[3-(aminomethyl)phenyl]-3-t-butyl-1H-pyrazol-5-yl}-3-(4-chloro-phenyl)urea as the HCl salt (78 mg, 82% yield). ¹H NMR (DMSO-d₆): δ 9.96 (s, 1H), 8.85 (s, 1H), 8.42 (br s, 3H), 7.72 (s, 1H), 7.56-7.55 (m, 2H), 7.48-7.45 (m, 3H), 7.32-7.30 (m, 2H), 6.41 (s, 1H), 4.16-4.12 (m, 2H), 1.29 (s, 9H); MS (ESI) m/z: 398.3 (M+H⁺), 400.2 (M+2+H⁺).

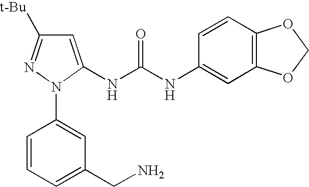

### Example 288

Using the same procedure as for Example 281, Example 254 (0.100 g, 0.25 mmol) was reduced to afford 1-{1-[3-(aminomethyl)phenyl]-3-t-butyl-1H-pyrazol-5-yl}-3-(benzo-[d][1,3]dioxol-5-yl)urea as its TFA salt as a white powder (67.1 mg, 66% yield). ¹H NMR (DMSO-d₆): δ 8.96 (s, 1H), 8.41 (s, 1H), 8.19 (br s, 3H), 7.67-7.47 (m, 4H), 7.15 (s, 1H), 6.82-6.80 (m, 1H), 6.71-6.69 (m, 1H), 6.37 (s, 1H), 5.96 (s, 2H), 4.13-4.12 (m, 2H), 1.28 (s, 9H); MS (ESI) m/z: 408.3 (M+H⁺).

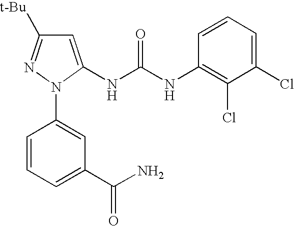

### Example 289

Example 256 (80 mg, 0.19 mmol) was suspended in conc. HCl (0.93 ml) and briskly stirred. More conc. HCl (1 ml) was added several times to maintain good stirring and keep the solids wetted. The reaction was stirred at RT 5 h and 24 h at 40° C. The reaction was cooled to RT, diluted with H₂O and EtOAc and the layers separated. The aqueous was extracted with EtOAc (2×). Solids in the aqueous layer were collected by filtration, rinsed sparingly with H₂O and dried. These solids were suspended in MeOH, then collected by filtration, rinsed with MeOH and washed with EtOAc to afford 1-[3-t-butyl-1-(3-carbamoylphenyl)-1H-pyrazol-5-yl]-3-(2,3-dichlorophenyl)urea as a white solid (47.3 mg, 57% yield). ¹H NMR (DMSO-d₆): δ 9.81 (br s, 1H), 8.99 (br s, 1H), 8.25 (br s, 1H), 8.08 (s, 1H), 7.99-7.97 (m, 1H), 7.90-7.87 (m, 1H), 7.75-7.71 (m, 1H), 7.60-7.57 (m, 1H), 7.49 (br s, 1H), 7.32-7.28 (m, 2H), 6.38 (br s, 1H), 1.29 (s, 9H); MS (ESI) m/z: 446.3 (M+H⁺), 448.3 (M+2+H⁺).

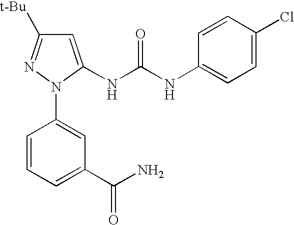

### Example 290

Using the same procedure as Example 289, Example 255 (0.174 g, 0.442 mmol) was transformed to provide 1-[3-t-butyl-L-(3-carbamoylphenyl)-1H-pyrazol-5-yl]-3-(4-chlorophenyl)urea as a pale yellow fluffy solid (47.4 mg). ¹H NMR (DMSO-d₆): δ 9.13 (s, 1H), 8.51 (s, 1H), 8.11 (br s, 1H), 8.02-8.01 (m, 1H), 7.92-7.89 (m, 1H), 7.68-7.66 (m, 1H), 7.62-7.58 (m, 1H), 7.52 (br s, 1H), 7.44-7.42 (m, 2H), 7.31-7.29 (m, 2H), 6.39 (s, 1H), 1.29 (s, 9H; MS (ESI) m/z: 412.3 (M+H⁺), 414.2 (M+2+H⁺).

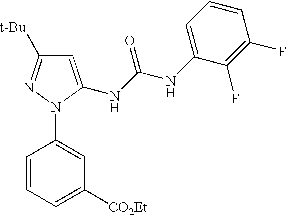

### Example 291

Using the same procedure as for Example 202, Example SS (143 mg, 0.5 mmol) and 2,3-difluorophenylamine (67 mg, 0.5 mmol) were combined to afford ethyl 3-{3-t-butyl-5-[3-(2,3-difluorophenyl)ureido]-1H-pyrazol-1-yl}benzoate (50 mg, 23% yield).

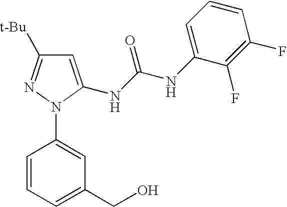

### Example 292

Using the same procedure as for Example 200, Example 291 (45 mg, 0.10 mmol) was reduced to afford 1-{3-t-butyl-1-[3-(hydroxymethyl)phenyl]-1H-pyrazol-5-yl}-3-(2,3-difluorophenyl)urea (30 mg, 75% yield). ¹H-NMR (300 MHz, DMSO-d₆): δ 9.08 (s, 1H), 8.85 (s, 1H), 7.88 (t, J=7.5 Hz, 1H), 7.48-7.42 (m, 2H), 7.33 (d, J=7.5 Hz, 2H), 7.13-6.95 (m, 2H), 6.36 (s, 1H), 4.55 (s, 1H), 1.24 (s, 9H); MS (ESI) m/z: 401 (M+H⁺).

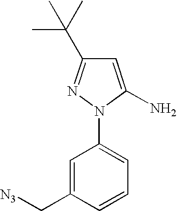

### Example III

To a suspension of LiAlH₄ (5.28 g, 139.2 mmol) in THF (1000 mL) was added Example SS (20.0 g, 69.6 mmol) in portions at 0° C. under N₂. The reaction mixture was stirred for 5 h, quenched with 1 N HCl at 0° C. and the precipitate was filtered, washed by EtOAc and the filtrate evaporated to yield [3-(5-amino-3-t-butyl-1H-pyrazol-1-yl)phenyl]methanol (15.2 g, 89%). ¹H NMR (DMSO-d₆): 7.49 (s, 1H), 7.37 (m, 2H), 7.19 (d, J=7.2 Hz, 1H), 5.35 (s, 1H), 5.25 (t, J=5.6 Hz, 1H), 5.14 (s, 2H), 4.53 (d, J=5.6 Hz, 2H), 1.19 (s, 9H); MS (ESI) m/z: 246.19 (M+H⁺).

The crude material from the previous reaction (5.0 g, 20.4 mmol) was dissolved in dry THF (50 mL) and SOCl₂ (4.85 g, 40.8 mmol), stirred for 2 h at RT, concentrated in vacuo to yield 3-t-butyl-1-(3-chloromethylphenyl)-1H-pyrazol-5-amine (5.4 g), which was added to NaN₃ (3.93 g, 60.5 mmol) in DMF (50 mL). The reaction mixture was heated at 30° C. for 2 h, poured into H₂O (50 mL), and extracted with CH₂Cl₂. The organic layers were combined, dried (MgSO₄), filtered and concentrated in vacuo to yield crude 3-t-butyl-1-[3-(azidomethyl)phenyl]-1H-pyrazol-5-amine (1.50 g, 5.55 mmol).

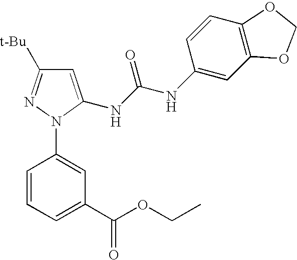

### Example 293

Using the same procedure as for Example 201, Example SS (500 mg, 1.74 mmol) and 5-isocyanato-benzo[1,3]dioxole (290 mg, 1.8 mmol) were combined to afford ethyl 3-{5-[3-(benzo[d][1,3]dioxo-5-yl)ureido]-3-t-butyl-1H-pyrazol-1-yl}benzoate (320 mg, 41% yield). ¹H NMR (300 MHz, DMSO-d₆): δ 8.73 (s, 1H), 8.34 (s, 1H), 8.03 (s, 1H), 7.92 (d, J=8.4 Hz, 1H), 7.78 (d, J=7.8 Hz, 1H), 7.63 (t, J=7.8 Hz, 1H), 7.09 (s, 1H), 6.76 (d, J=8.1 Hz, 2H), 6.68 (d, J=8.4 Hz, 1H), 6.32 (s, 1H), 5.92 (s, 2H), 4.29 (q, J=6.9 Hz, 2H), 1.28 (s, 9H), 1.26 (t, J=6.9 Hz, 3H); MS (ESI) m/z: 451 (M+H⁺).

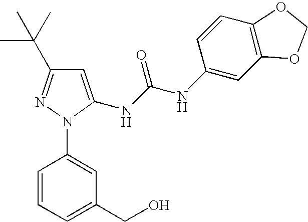

### Example 294

Using the same procedure as for Example 200, Example 293 (100 mg, 0.22 mmol) was reduced to afford 1-(benzo[d][1,3]dioxol-5-yl)-3-(3-t-butyl-1-(3-(hydroxymethyl)phenyl)-1H-pyrazol-5-yl)urea (50 mg, 56% yield). ¹H NMR (300 MHz, CD₃OD): δ 7.52-7.47 (m, 4H), 7.02 (s, 1H), 6.65-6.69 (m, 2H), 6.41 (s, 1H), 5.89 (s, 2H), 4.69 (s, 2H), 1.33 (s, 9H); MS (ESI) m/z: 409 (M+H⁺).

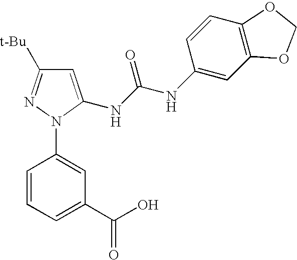

### Example 295

To a solution of Example 293 (50 mg, 0.11 mmol) in THF (10 mL) was added a aqueous solution of LiOH (2 N, 5 mL) at 0° C. The mixture was stirred at RT overnight. After removal of the solvent, the residue was dissolved in water, then acidified to pH=4.0 with 1 N of HCl. The mixture was extracted with CH₂Cl₂ (3×50 mL) and t the combined organic extracts were washed with brine, dried (Na₂SO₄), filtered, concentrated and purified via preparative HPLC to afford 3-{5-[3-(benzo[d][1,3]dioxol-5-yl)ureido]-3-t-butyl-1H-pyrazol-1-yl}benzoic acid (30 mg, 65% yield). ¹H NMR (300 MHz, CD₃OD): δ 8.15 (s, 1H), 8.08 (d, J=7.8 Hz, 1H), 7.75 (d, J=8.4 Hz, 1H), 7.63 (t, J=7.8 Hz, 1H), 6.99 (s, 1H), 6.67-6.62 (m, 2H), 6.39 (s, 1H), 5.89 (s, 2H), 1.33 (s, 9H); MS (ESI) m/z: 423 (M+H⁺).

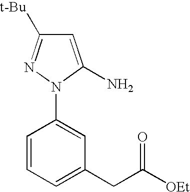

### Example JJJ

A mixture of 1-(3-nitro-phenyl)-ethanone (82.5 g, 0.5 mol), toluene-4-sulfonic acid (3 g) and sulfur (32 g, 1.0 mol) in morpholine (100 mL) was heated to reflux for 3 h. After removal of the solvent, the residue was dissolved in dioxane (100 mL), treated with conc. HCl (100 mL), then heated to reflux for 5 h. After removal of the solvent, the residue was extracted with EtOAc (3×150 mL). The combined organic layers were washed with brine, dried over anhydrous sodium sulfate and filtered. The filtrate was evaporated to the residue under reduced pressure. The residue was dissolved in ethanol (250 mL) and then added SOCl₂ (50 mL). The mixture was heated to reflux for 2 h. After removal of the solvent, the residue was extracted with ethyl acetate (3×150 mL). The combined organic extracts were washed with brine, dried (Na₂SO₄), filtered, concentrated and purified via column chromatography to afford 40 g of (3-nitro-phenyl)-acetic acid ethyl ester. ¹H-NMR (300 MHz, DMSO-d₆): δ 8.17 (s, 1H), 8.11 (d, J=7.2 Hz, 1H), 7.72 (d, J=7.2 Hz, 1H), 7.61 (t, J=7.8 Hz, 1H), 4.08 (q, J=7.2 Hz, 2H), 3.87 (s, 2H), 1.17 (t, J=7.2 Hz, 3H)

A mixture of (3-nitro-phenyl)-acetic acid ethyl ester (31.4 g, 0.15 mol) and Pd/C (3.5 g) in methanol (200 mL) was stirred under 40 psi of H₂ a tRT for 2 h, then filtered. After removal of the solvent, 25 g of 3-(3-amino-phenyl)-acetic acid ethyl ester was obtained (93%), which was used without further purification. MS (ESI): m/z: 180 (M+H⁺)

To a solution of 3-(3-amino-phenyl)-acetic acid ethyl ester (18 g, 0.1 mol) in concentrated HCl (200 mL) was added an aqueous solution (20 mL) of NaNO₂ (6.9 g, 0.1 mmol) at 0° C. and the resulting mixture was stirred for 1 h. A solution of SnCl₂.2H₂O (44.5 g, 0.2 mmol) in concentrated HCl (200 mL) was then added at 0° C. The reaction solution was stirred for 2 h at RT. The reaction mixture was adjusted to pH=8.0 with 2 N NaOH and extracted with EtOAc (3×150 mL). The combined organic extracts were washed with brine, dried (Na₂SO₄), filtered, and concentrated to yield 15 g of 3-(3-hydrazino-phenyl)-acetic acid ethyl ester (77%) as a white solid, which was used without further purification; MS (ESI) m/z: 194 (M+H⁺).

A mixture of 3-(3-hydrazino-phenyl)-acetic acid ethyl ester (9.7 g, 50 mmol) and 4,4-dimethyl-3-oxo-pentanenitrile (6.9 g, 55 mol) in ethanol (150 mL) was heated to reflux overnight. The reaction solution was evaporated under vacuum. The residue was purified via column chromatography to give 13 g 3-[3-(5-amino-3-t-butyl-pyrazol-1-yl)-phenyl]-acetic acid ethyl ester (87%). ¹H-NMR (300 MHz, DMSO-d₆); 7.44 (s, 1H), 7.43 (d, J=8.1 Hz, 1H), 7.35 (t, J=7.5 Hz, 1H), 7.12 (d, J=7.5 Hz, 1H), 5.35 (s, 1H), 5.17 (br s, 2H), 4.05 (q, J=6.9 Hz, 2H), 3.69 (s, 2H), 1.18 (s, 9H), 1.16 (t, J=6.9 Hz, 3H); MS (ESI) m/z: 302 (M+H⁺)

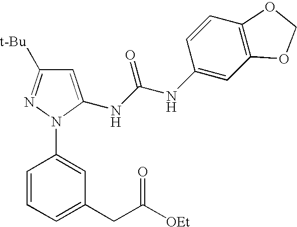

### Example 296

To a solution of Example JJJ (1.0 g, 3.32 mmol) and Et₃N (606 mg, 6.0 mmol) in THF (50 mL) was added 5-isocyanato-benzo[1,3]dioxole (570 mg, 3.5 mmol) in THF (5.0 mL) at 0° C. The mixture was stirred at RT for 3 h, and then poured into water (100 mL). The mixture was extracted with CH₂Cl₂ (3×). The combined organic extracts were washed with brine, dried (Na₂SO₄), filtered, concentrated and purified via column chromatography to afford ethyl 2-(3-{5-[3-(benzo[d][1,3]dioxol-5-yl)ureido]-3-t-butyl-1H-pyrazol-1-yl}phenyl)-acetate (950 mg, 62% yield). ¹H NMR (300 MHz, DMSO-d₆): δ 8.84 (s, 1H), 8.28 (s, 1H), 7.48-7.34 (m, 3H), 7.27 (d, J=8.4 Hz, 1H), 7.11 (s, 1H), 6.76 (d, J=7.8 Hz, 1H), 6.66 (d, J=7.8 Hz, 1H), 6.31 (s, 1H), 5.92 (s, 2H), 4.04 (q, J=7.2 Hz, 2H), 3.73 (s, 2H), 1.23 (s, 9H), 1.15 (t, J=7.8 Hz, 3H); MS (ESI) m/z: 465 (M+H⁺).

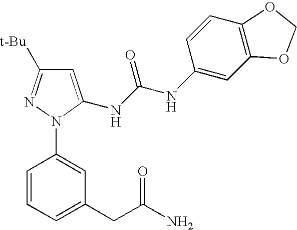

### Example 297

To a solution of Example 296 (150 mg, 0.20 mmol) in THF (5 mL) was added aqueous NH₃ (10 mL, 25%) at RT. The mixture was heated to 70° C. for 5 h, and then concentrated, and the residue was purified by preparative HPLC to afford 1-{1-[3-(2-amino-2-oxoethyl)phenyl]-3-t-butyl-1H-pyrazol-5-yl}-3-(benzo[d][1,3]dioxol-5-yl)urea (70 mg, 80% yield). ¹H-NMR (300 MHz, DMSO-d₆): δ 8.85 (s, 1H), 8.31 (s, 1H), 7.51-7.26 (m, 5H), 7.11 (s, 1H), 6.90 (br s, 1H), 6.76 (d, J=8.4 Hz, 1H), 6.65 (d, J=7.8 Hz, 1H), 6.32 (s, 1H), 5.92 (s, 2H), 3.42 (s, 2H), 1.23 (s, 9H); MS (ESI) m/z: 436 (M+H⁺).

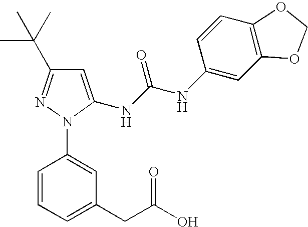

### Example 298

Using the same procedure as for Example 203, Example 296 (500 mg, 1.1 mmol) was saponified to afford 2-(3-(5-[3-(benzo[1,3]dioxol-5-yl)ureido]-3-t-butyl-1H-pyrazol-1-yl)phenyl)acetic acid (450 mg, 94% yield). ¹H NMR (300 MHz, CD₃OD): δ 7.52-7.35 (m, 4H), 7.01 (s, 1H), 6.70-6.61 (m, 2H), 6.40 (s, 1H), 5.89 (s, 2H), 3.72 (s, 2H), 1.32 (s, 9H); MS (ESI) m/z: 437 (M+H⁺).

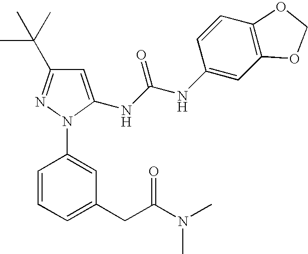

### Example 299

A mixture of Example 298 (500 mg, 1.1 mmol), dimethylamine (135 mg, 3.0 mmol), DIEA (390 mg, 3.0 mmol) and PyBop (780 mg, 1.5 mmol) in THF (50 mL) was stirred at RT overnight. After removal of the solvent, the residue was dissolved in CH₂Cl₂ (100 mL) and washed with 1.0 N HCl and brine. The combined organic extracts were washed with brine, dried (Na₂SO₄), filtered, concentrated and purified via column chromatography to afford 1-(benzo[d][1,3]dioxol-5-yl)-3-{3-t-butyl-1-[3-(2-(dimethylamino)-2-oxoethyl]phenyl}-1H-pyrazol-5-yl)urea (470 mg, 92% yield). ¹H NMR (300 MHz, DMSO-d₆): δ 9.00 (s, 1H), 8.39 (s, 1H), 7.43-7.35 (m, 3H), 7.21 (d, J=7.2 Hz, 1H), 7.11 (s, 1H), 6.76 (d, J=8.4 Hz, 1H), 6.65 (d, J=7.8 Hz, 1 H), 6.30 (s, 1H), 5.92 (s, 2H), 3.73 (s, 2H), 2.98 (s, 3H), 2.78 (s, 3H), 1.23 (s, 9H); MS (ESI) m/z: 464 (M+H⁺).

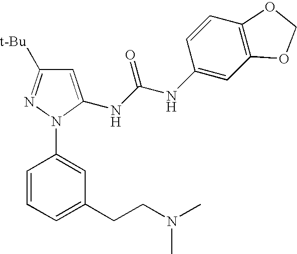

### Example 300

To a solution of Example 299 (150 mg, 0.32 mmol) in THF (20 mL) was added LAH powder (23 mg, 0.6 mmol) at RT under N₂. The mixture was heated to reflux for 3 h, and then quenched with water and aqueous NaOH. The suspension was filtered and the filtrate was concentrated and purified by preparative HPLC to afford the TFA salt. The mixture of TFA salt in MeCN/H₂O (50 mL) was basified to pH=10.0 with an aqueous solution of 1.0 N Na₂CO₃. After lyophylization, the residue was dissolved in THF, filtered and the filtrate was adjusted to pH=6.0 with 1.0 N HCl/MeOH (2.0 mL) and then concentrated to afford 1-(benzo[d][1,3]dioxol-5-yl)-3-{3-t-butyl 1-(3-[2-(dimethylamino)ethyl]phenyl}-1H-pyrazol-5-yl)urea (95 mg, 66% yield). ¹H NMR (300 MHz, CD₃OD): δ 7.56-7.39 (m, 4H), 6.99 (s, 1H), 6.71 (d, J=8.4 Hz, 1H), 6.62 (d, J=7.8 Hz, 1H), 6.38 (s, 1H), 5.89 (s, 2H), 3.41 (t, J=7.2 Hz, 2H), 3.13 (t, J=7.2 Hz, 2H), 2.90 (s, 6H), 1.34 (s, 9H); MS (ESI) m/z: 450 (M+H⁺).

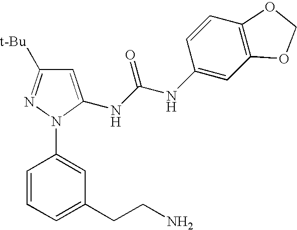

### Example 301

Using the same procedure as for Example 281, Example 297 (200 mg, 0.45 mmol) was reduced to yield 1-{1-[3-(2-aminoethyl)phenyl]-3-t-butyl-1H-pyrazol-5-yl}-3-(benzo[d][1,3]dioxol-5-yl)urea as the hydrochloride salt (80 mg, 40% yield). ¹H NMR (300 MHz, DMSO-d₆): δ 9.37 (s, 1H), 8.65 (s, 1H), 7.92 (br s, 3H), 7.52-7.47 (m, 3H), 7.28 (d, J=7.8 Hz, 1H), 7.02 (s, 1H), 6.65-6.69 (m, 2H), 6.31 (s, 1H), 5.92 (s, 2H), 3.13-3.07 (m, 2H), 2.96-2.88 (m, 2H), 1.24 (s, 9H); MS (ESI) m/z: 422 (M+H).

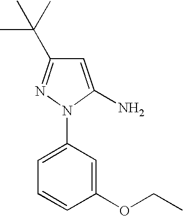

### Example KKK

m-Phenetodine (1.51 g, 11.0 mmol) was dissolved in concentrated HCl (16 mL) and the solution was stirred in an ice-salt bath. Sodium nitrite (0.76 g, 11.0 mmol) was dissolved in water (14 mL) and chilled to 0° C., then added dropwise maintaining an internal reaction temperature of 0° C. The reaction mixture was stirred at 0-5° C. for 1 h. Reducing agent, tin chloride dehydrate (5.71 g, 25.3 mmol) was dissolved in conc. HCl (10 mL) and chilled to 0° C. and slowly added to the reaction mixture and stirred at 0-5° C. for 1 h. The reaction mixture was filtered and the solid washed with chilled 6N HCl. The solid was dissolved in water and lyophilized under reduced pressure to obtain 1-(3-ethoxyphenyl)-hydrazine HCl salt as a brown powder (1.61 g, 77% yield), which was used without further purification.

To a solution of 1-(3-ethoxyphenyl)-hydrazine (300 mg, 1.6 mmol) in toluene (5 mL) was added pivaloylacetonitrile (200 mg, 1.6 mmol). The reaction mixture was heated to reflux for 5 h. The reaction mixture was filtered and washed with hexane to obtain 3-t-butyl-1-(3-ethoxyphenyl)-1H-pyrazole-5-amine HCl salt as an orange solid (320 mg, 68% yield). ¹H NMR (DMSO-d₆): δ 7.47 (t, J=8.0 Hz, 1H), 7.0-7.4 (m, 3H), 5.60 (s, 1H), 4.12 (q, J=7.0 Hz, 2H), 1.35 (t, J=7.0 Hz, 3H), 1.28 (s, 9H); MS (EI) m/z: 260 (M+H⁺).

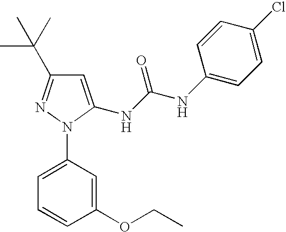

### Example 302

To a solution of Example KKK (70 mg, 0.14 mmol) in THF (2 mL) was added pyridine (38 mL, 0.28 mmol) and 4-chlorophenyl isocyanate (36 mg, 0.14 mmol). The reaction mixture was stirred at 40° C. for 12 h. The reaction mixture was concentrated under reduced pressure and the residue was purified by column chromatography to yield 1-[3-t-butyl-1-(3-ethoxyphenyl)-1H-pyrazol-5-yl]-3-(4-chlorophenyl)urea as a white powder (10 mg, 10% yield). ¹H NMR (CDCl₃): δ 7.34 (br s, 1H), 7.21 (m, 5H), 6.93 (m, 2H), 6.88 (br s, 1H), 6.83 (dd, J=1.8, and 8.6 Hz, 1H), 6.39 (s, 1H), 5.60 (s, 1H), 3.94 (q, J=7.0 Hz, 2H), 1.36 (t, J=7.0 Hz, 3H), 1.34 (s, 9H); MS (EI) m/z: 413 (M+H⁺).

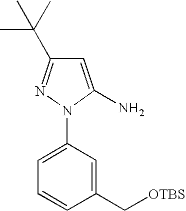

### Example LLL

To a solution of CuI (1 mol %), 1,10-phenanthroline (10 mol %), Cs₂CO₃ (9.8 g, 30 mmol) and DMF (20 mL) was added t-butyl carbazate (3.4 g, 25 mmol), 3-iodobenzyl alcohol (5.0 g, 21 mmol). The reaction mixture was heated at 80° C. for 2 h. The reaction mixture was filtered through a pad of silica gel and the filtrate was evaporated under reduced pressure to obtain crude product, 1-Boc-1-(3-carbinol)phenylhydrazine as yellow oil. The product was used for the next reaction without further purification.

To a solution of 1-Boc-1-(3-carbinol)phenylhydrazine (2.0 g, 8.4 mmol) in absolute ethanol (30 mL) at RT was added concentrated HCl (3.5 mL, 42 mmol). The reaction mixture was stirred at 60° C. for 30 min. Pivaloylacetonitrile (1.3 g, 10 mmol) was added into the reaction mixture, which was heated at 90° C. for 3 h. The solvent was evaporated under reduced pressure and the residue was dissolved in water and lyophilized to obtain the crude product [3-(5-amino-3-t-butyl-1H-pyrazol-1-yl)phenyl]methanol as the HCl salt. The product was used for the next step without further purification. ¹H-NMR (DMSO-d₆): δ 7.4-7.6 (m, 4H), 5.62 (br s, 1H), 4.59 (s, 2H), 1.29 (s, 9H).

To a solution of [3-(5-amino-3-t-butyl-1H-pyrazol-1-yl)phenyl]methanol hydrochloride salt (2.0 g, 7.1 mmol) in DMP (20 mL) was added imidazole (2.7 g, 39 mmol) and TBSCl (2.1 g, 14 mmol), which was stirred at RT for 8 h. The reaction mixture was quenched with water and extracted with EtOAc (3×). Organic extracts were washed with NaHCO₃, H₂O and 10% LiCl solution. The combined organic extracts were washed with brine, dried (Na₂SO₄), filtered, concentrated and purified via column chromatography to yield 3-t-butyl-1-(3-[(t-butylmethylsilyloxy)methyl]phenyl)-1H-pyrazol-5-amine in 36% yield (for three steps): ¹H-NMR (CDCl₃): δ 7.3-7.6 (m, 4H), 5.54 (s, 1H), 4.80 (s, 2H), 1.34 (s, 9H), 0.97 (s, 9H), 0.13 (s, 6H); MS (EI) m/z: 360 (M+H⁺).

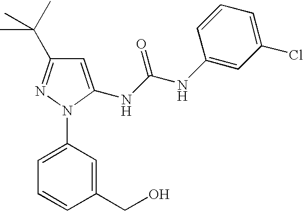

### Example 303

To a solution of Example LLL (100 mg, 0.18 mmol) in THF (2 mL) was added pyridine (45 mL, 0.56 mmol) and 3-chlorophenyl isocyanate (43 mg, 0.18 mmol). The reaction mixture was stirred at RT for 20 min, heated until all solids were dissolved, and stirred at RT for 4 h. The reaction mixture was concentrated under reduced pressure to yield 1-(3-t-butyl-{-3-[(t-butyldimethylsilyloxy)methyl]phenyl}-1H-pyrazol-5-yl)-3-(3-chlorophenyl)urea (62 mg, 43% yield).

To a solution of 1-(3-t-butyl-1-{3-[(t-butyldimethylsilyloxy)methyl]phenyl}-1H-pyrazol-5-yl)-3-(3-chlorophenyl)urea (120 mg, 0.12 mmol) in THF (2 mL) was added TBAF (1.0 M, 0.13 mL, 0.13 mmol). The reaction mixture was stirred at RT for 2.5 h. The solvent was removed under reduced pressure. EtOAc was added into the residue followed by 1N-HCl (5 drops). The combined organic extracts were washed with brine, dried (Na₂SO₄), filtered, concentrated and purified via column chromatography to yield 1-(3-t-butyl-1-(3-hydroxymethyl)phenyl)-1H-pyrazol-5-yl)-3-(3-chlorophenyl)urea as a white powder (34 mg, 71% yield). ¹H-NMR (CDCl₃): δ 8.11 (s, 1H), 7.34 (t, J=2.0 Hz, 1H), 7.05-7.25 (m, 7H), 6.99 (dt, J=1.3, and 7.8 Hz, 1H), 6.39 (s, 1H), 4.39 (s, 2H), 1.33 (s, 9H); MS (EI) m/z: 399 (M+H⁺).

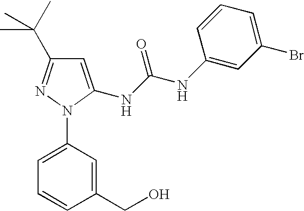

### Example 304

Using the same procedure as for Example 303, Example LLL (100 mg, 0.28 mmol) and 3-bromophenyl isocyanate (55 mg, 0.28 mmol) were combined to yield 1-{3-t-butyl-1-[3-(hydroxymethyl)phenyl]-1H-pyrazol-5-yl}-3-(3-bromophenyl)urea as a white powder (19 mg, 15% yield). ¹H-NMR (CDCl₃): δ 8.17 (s, 1H), 7.47 (t, J=1.8 Hz, 1H), 7.34 (s, 1H), 7.00-7.25 (m, 7H), 6.39 (s, 1H), 4.37 (s, 2H), 1.32 (s, 9H); MS (EI) m/z: 443 and 445 (M⁺ and M⁺+2H⁺).

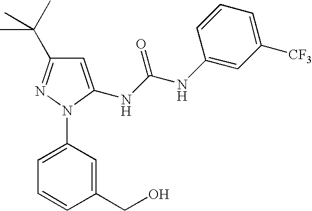

### Example 305

Using the same procedure as for Example 303, Example KKK (100 mg, 0.28 mmol) and 3-(trifluoromethyl)phenyl isocyanate (52 mg, 0.28 mmol) were combined to yield 1-{3-t-butyl-1-[3-(hydroxymethyl)phenyl]-1H-pyrazol-5-yl}-3-(3-(trifluoromethyl)phenyl)urea as a white powder (42 mg, 35% yield). ¹H-NMR (CDCl₃): δ 8.21 (bs, 1H), 7.64 (t, J=1.8 Hz, 1H), 7.1-7.5 (m, 8H), 6.51 (s, 1H), 4.56 (s, 2H), 1.37 (s, 9H); MS (EI) m/z: 433 (M+H⁺).

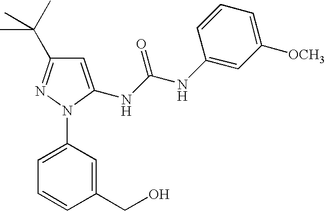

### Example 406

Using the same procedure as for Example 303, Example LLL (100 mg, 0.28 mmol) and 3-methoxyphenyl isocyanate (41 mg, 0.28 mmol) were combined to yield 1-{3-t-butyl-1-[3-(hydroxymethyl)phenyl]-1H-pyrazol-5-yl}-3-(3-methoxyphenyl)urea as a white powder (34 mg, 31% yield). ¹H-NMR (CDCl₃): δ 7.86 (br s, 1H), 7.34 (br s, 1H), 7.31 (d, J=7.6 Hz, 1H), 7.18 (d, J=6.4 Hz, 1H), 7.15 (t, J=8.2 Hz, 1H), 7.00 (t, J=2.1 Hz, 1H), 6.80 (dd, J=1.1, and 8.0 Hz, 1H), 6.62 (dd, J=2.3, and 8.3 Hz, 1H), 6.50 (s, 1H), 4.56 (s, 2H), 3.76 (s, 3H), 1.37 (s, 9H); MS (EI) m/z: 395 (M+H⁺).

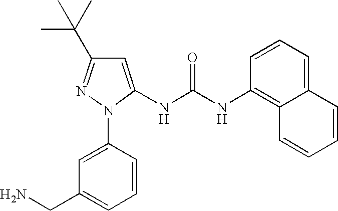

### Example 307

Example III was dissolved in dry THF (10 mL) and added to a THF solution (10 mL) of 1-isocyano naphthalene (1.13 g, 6.66 mmol) and pyridine (5.27 g, 66.6 mmol) at RT. The reaction mixture was stirred for 3 h, quenched with H₂O (30 mL), and the resulting precipitate filtered and washed with 1N HCl and ether to yield 1-[2-(3-azidomethyl-phenyl)-5-t-butyl-2H-pyrazol-3-yl]-3-naphthalen-1-yl-urea (2.4 g, 98%) as a white solid.

The crude material from the previous reaction and Pd/C (0.4 g) in THF (30 mL) was hydrogenated under 1 atm at RT for 2 h. The catalyst was removed by filtration and the filtrate concentrated in vacuo to yield 1-(3-t-butyl-1-(3-(aminomethyl)phenyl)-1H-pyrazol-5-yl)-3-(naphthalen-1-yl)urea (2.2 g, 96%) as a yellow solid. ¹H NMR (DMSO-d₆): 9.02 (s, 1H), 7.91 (d, J=7.2 Hz, 1H), 7.89 (d, J=7.6 Hz, 2H), 7.67-7.33 (m, 9H), 6.40 (s, 1H), 3.81 (s, 2H), 1.27 (s, 9H); MS (ESI) m/z: 414 (M+H).

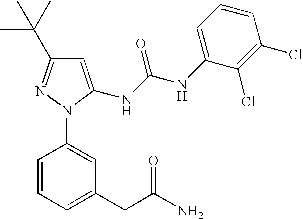

### Example 308

Using the same procedure as for Example 201, Example AAA (136 mg, 0.5 mmol) and added 1,2-dichloro-3-isocyanatobenzene (98 mg, 0.5 mmol) were combined to afford 1-{1-[3-(2-amino-2-oxoethyl)phenyl]-3-t-butyl-1H-pyrazol-5-yl}-3-(2,3-dichlorophenyl)urea (60 mg, 26% yield). ¹H NMR (300 MHz, DMSO-d₆): δ 9.23 (s, 1H), 8.75 (s, 1H), 8.04 (m, 1H), 7.50 (br s, 1H), 7.45-7.25 (m, 7H), 6.90 (br s, 1H), 6.36 (s, 1H), 3.42 (s, 2H), 1.24 (s, 9H); MS (ESI) m/z: 459, (M+H⁺).

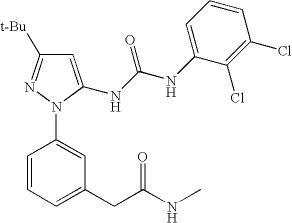

### Example 310

To a solution of Example 248 (100 mg, 0.20 mmol) in anhydrous MeOH (10 mL) was added a solution of CH₃NH₂ (5 mL, 25%) in MeOH at RT. The mixture was heated to 50° C. for 3 h. After removal of the solvent, the residue was purified by preparative HPLC to afford 1-{3-t-butyl-1-[3-(methylamino)-2-oxoethyl]phenyl}-1H-pyrazol-5-yl}-3-(2,3-dichlorophenyl)urea (70 mg, 74% yield). ¹H-NMR (300 MHz, DMSO-d₆): δ 9.40 (br s, 1H), 8.84 (s, 1H), 8.04-8.02 (m, 2H), 7.41-7.33 (m, 3H), 7.27-7.25 (m, 3H), 6.34 (s, 1H), 3.44 (s, 2H), 3.34 (s, 3H), 1.24 (s, 9H); MS (ESI) m/z: 474 (M+H⁺).

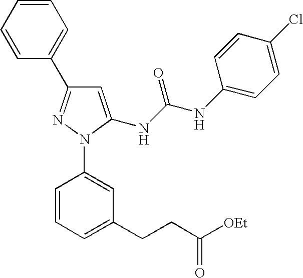

### Example 311

To a solution of commercially available 3-oxo-3-phenyl-propionitrile (1.45 g, 10.0 mmol) and ethanol (690 mg, 15.0 mmol) in CH₂Cl₂ (50 mL) was bubbled HCl gas at 0° C. for 1 h. The resulting mixture was warmed to RT and stirred overnight. After removal of the solvent, the residue was washed with Et₂O to afford 1.6 g of 3-oxo-3-phenyl-propionimidic acid ethyl ester hydrochloride, which was used to the next reaction without further purification. MS (ESI) m/z: 228 (M+H⁺).

To a solution of 3-oxo-3-phenyl-propionimidic acid ethyl ester hydrochloride (1.5 g, 6.6 mmol) and Et₃N (2.02 g, 20 mmol) in THF (50 mL) was added 1-chloro-4-isocyanato-benzene (1.1 g, 7.2 mmol) at 0° C. The resulting mixture was stirred at RT overnight, then poured to water (100 mL). The mixture was extracted with CH₂Cl₂ (3×100 mL). The combined organic extracts were washed with brine, dried (Na₂SO₄), filtered, concentrated and purified via column chromatography to afford 2.0 g of 1-(4-chloro-phenyl)-3-(1-ethoxy-3-oxo-3-phenyl-propenyl)-urea MS (ESI) m/z: 345 (M+H⁺).

A mixture of 3-(3-hydrazino-phenyl)-propionic acid ethyl ester (See Example NN, 500 mg, 2.05 mmol) and 1-(4-chloro-phenyl)-3-(1-ethoxy-3-oxo-3-phenyl-propenyl)-urea (688 mg, 2.0 mol) in ethanol (100 nm) was stirred at RT for 3 h. After removal of the solvent, the residue was purified by column chromatography to yield 700 mg of 3-(3-{5-[3-(4-chloro phenyl)-ureido]-3-phenyl-pyrazol-1-yl}-phenyl)-propionic acid ethyl ester. ¹H NMR (400 MHz, CD₄O-d₆): 7.83 (d, J=7.6 Hz, 2H), 7.51-7.33 (m, 9H), 7.26 (d, J=8.8 Hz, 2H), 6.89 (s, 1H), 4.09 (q, J=7.2 Hz, 2H), 3.03 (t, J=7.6 Hz, 2H), 2.69 (t, J=7.6 Hz, 2H), 1.20 (t, J=7.2 Hz, 3H). MS (ESI) m/z: 489 (M+H⁺).

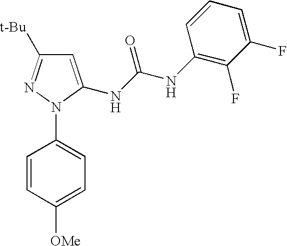

### Example 312

Using the same procedure as for Example 202, Example YY (123 mg, 0.5 mmol) and 1-fluoro-2,3-difluorophenylamine (65 mg, 0.5 mmol) were combined to afford 1-[3-t-butyl-1-(4-methoxyphenyl)-1H-pyrazol-5-yl]-3-(2,3-difluorophenyl)urea (65 mg, 32% yield). ¹H-NMR (300 MHz, DMSO-d₆): 8.9.08 (s, 1H), 8.77 (s, 1H), 7.90 (t, J=7.2 Hz, 1H), 7.37 (d, J=9.0 Hz, 2H), 7.13-6.95 (m, 4H), 6.33 (s, 1H), 3.79 (s, 3H), 1.23 (s, 9H); MS (ESI) m/z: 401 (M+H⁺).

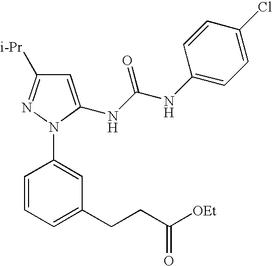

### Example 313

Using the same procedure as for Example 311, 4-methyl-3-oxo-pentanenitrile (from Example RRR, 1.11 g, 10.0 mmol) was transformed to 4-methyl-3-oxo-pentanimidic acid ethyl ester hydrochloride (1.0 g, 5.2 mmol), which was combined with 1-chloro-4-isocyanato-benzene (1.1 g, 7.2 mmol) to afford 1.5 g of 1-(4-chlorophenyl)-3-((E)-1-ethoxy-4-methyl-3-oxopent-1-enyl)urea (MS (ESI) m/z: 337 (M+H⁺)). This was combined with 3-(3-hydrazino-phenyl)-propionic acid ethyl ester (from Example EEE, 500 mg, 2.05 mmol) to yield 420 mg of ethyl 3-(3-(5-(3-(4-chlorophenyl)ureido)-3-isopropyl-1H-pyrazol-1-yl)phenyl)propanoate. ¹H NMR (400 MHz, CD₄O-d₄): 7.48 (t, J=8.0 Hz, 1H), 7.39-7.35 (m, 5H), 7.25 (d, J=8.8 Hz, 2H), 6.46 (s, 1H), 4.08 (q, J=7.2 Hz, 2H), 3.02-2.98 (m, 3H), 2.67 (t, J=7.6 Hz, 2H), 1.31 (d, J=6.8 Hz, 3H), 1.19 (t, J=7.2 Hz, 3H). MS (ESI) m/z: 455 (M+H⁺).

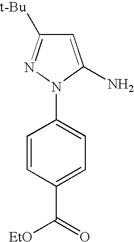

### Example MMM

Ethyl 4-(3-t-butyl-5-amino-1H-pyrazol-1-yl)benzoate (3.67 mmol) was prepared from ethyl 4-hydrazinobenzoate and pivaloylacetonitrile by the procedure of Regan, et al., J. Med. Chem., 45, 2994 (2002).

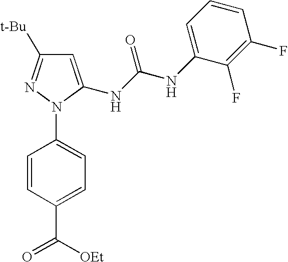

### Example 314

Using the same procedure as for Example 201, Example MMM (287 mg, 1.0 mmol), and 2,3-difluorophenylamine (134 mg, 1.0 mmol) were combined to afford ethyl 4-{3-t-butyl-5-[3-(2,3-difluorophenyl)ureido]-1H-pyrazol-1-yl}benzoate (250 mg, 57% yield).

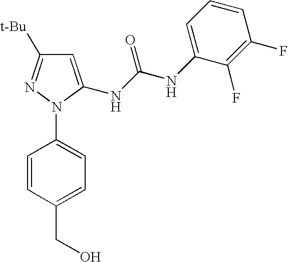

### Example 315

Using the same procedure as for Example 200, Example 314 (230 mg, 0.52 mmol) was reduced to afford 1-{3-t-butyl-1-[4-(hydroxymethyl)phenyl]-1H-pyrazol-5-yl}-3-(2,3-difluoro-phenyl)urea (160 mg, 80% yield). ¹H NMR (300 MHz, DMSO-d₆): δ 9.14 (s, 1H), 8.95 (s, 1H), 7.84-6.82 (m, 7H), 6.25 (s, 1H), 5.27 (t, J=5.7 Hz, 1H), 4.42 (br s, 2H), 1.14 (s, 9H); MS (ESI) m/z: 401 (M+H⁺).

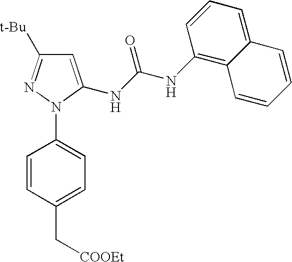

### Example 316

Using the same procedure as for Example 201, Example RR (5 g, 14.8 mmol) and 1-isocyanatonaphthalene (2.5 g, 15.0 mmol) I were combined to afford ethyl 2-(4-{3-t-butyl-5-[3-(naphthalen-1-yl)ureido]-1H-pyrazol-1-yl}phenyl)acetate (1.7 g, 24% yield). MS (ESI) m/z: 471 (M+H⁺).

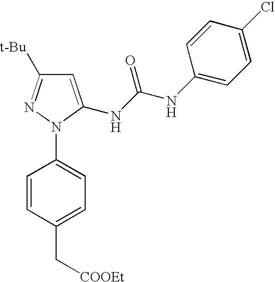

### Example 317

Using the same procedure as for Example 201, Example RR (5 g, 14.8 mmol) and 1-chloro-4-isocyanato-benzene (2.2 g, 15.0 mmol) were combined to afford ethyl 2-(4-{3-t-butyl-5-[3-(4-chlorophenyl)ureido]-1H-pyrazol-1-yl}phenyl)acetate (2.7 g, 40% yield). ¹H NMR (DMSO-d₆): δ 9.12 (s, 1H), 8.42 (s, 1H), 7.46-7.37 (m, 6H), 7.28 (d, J=8.1 Hz, 2H), 6.34 (s, 1H), 4.08 (q, J=7.2 Hz, 2H), 2.79 (t, J=7.2 Hz, 2H), 3.72 (s, 2H), 1.25 (s, 9H), 1.18 (t, J=7.2 Hz, 3H); MS (ESI) m/z: 455 (M+H⁺).

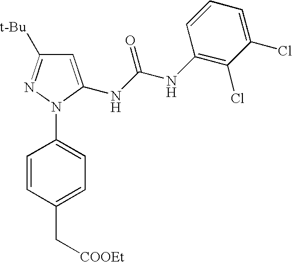

### Example 318

Using the same procedure as for Example 201, Example RR (5 g, 14.8 mmol) and 1,2-dichloro-3-isocyanatobenzene (2.8 g, 15.0 mmol) were combined to afford 2-(4-{3-t-butyl-5-[3-(2,3-dichlorophenyl)ureido]-1H-pyrazol-1-yl}phenyl)acetic acid (2.1 g, 29% yield). ¹H NMR (DMSO-d₆): δ 9.24 (s, 1H), 8.77 (s, 1H), 8.05 (m, 1H), 7.47-7.38 (m, 4H), 7.30-7.28 (m, 2H), 6.36 (s, 1H), 4.08 (q, J=7.2 Hz, 2H), 2.72 (s, 2H), 1.25 (s, 9H), 1.18 (t, J=7.2 Hz, 3H); MS (ESI) m/z: 489 (M+H⁺).

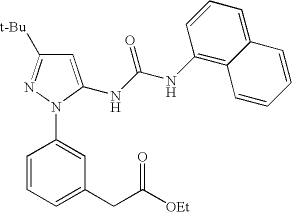

### Example 319

Using the same procedure as for Example 201, Example ZZ (5 g, 14.8 mmol) and 1-isocyanatonaphthalene (2.5 g, 15.0 mmol) were combined to afford ethyl 2-(3-{3-t-butyl-5-[3-(naphthalen-1-yl)ureido]-1H-pyrazol-1-yl}phenyl)acetate (1.5 g, 22% yield). MS (ESI) m/z: 471 (M+H⁺).

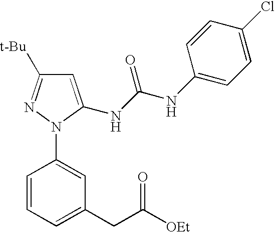

### Example 320

Using the same procedure as for Example 201, Example ZZ (5 g, 14.8 mmol) and 1-chloro-4-isocyanato-benzene (2.2 g, 15.0 mmol) were combined to afford ethyl 2-(3-{3-t-butyl-5-[3-(4-chlorophenyl)ureido]-1H-pyrazol-1-yl}phenyl)acetate (2.7 g, 40% yield). ¹H NMR (DMSO-d₆): δ 9.10 (s, 1H), 8.39 (s, 1H), 7.46-7.37 (m, 5H), 7.28-7.25 (m, 3H), 6.34 (s, 1H), 4.04 (q, J=7.2 Hz, 2H), 3.72 (s, 2H), 1.25 (s, 9H), 1.14 (t, J=7.2 Hz, 3H); MS (ESI) m/z: 455 (M+H⁺).

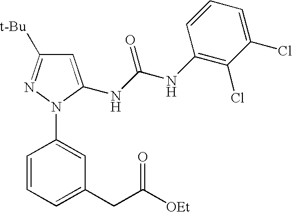

### Example 321

Using the same procedure as for Example 201, Example ZZ (5 g, 14.8 mmol) and 1,2-dichloro-3-isocyanato-benzene (2.8 g, 15.0 mmol) were combined to afford ethyl 2-(3-{3-t-butyl-5-[3-(2,3-dichlorophenyl)ureido]-1H-pyrazol-1-yl}phenyl)acetate (2.1 g, 29% yield). ¹H NMR (300 MHz, DMSO-d₆): δ 9.22 (s, 1H), 8.75 (s, 1H), 8.05 (m, 1H), 7.46-7.21 (m, 6H), 6.35 (s, 1H), 4.04 (q, J=7.2 Hz, 2H), 3.72 (s, 2H), 1.24 (s, 9H), 1.16 (t, J=7.2 Hz, 3H); MS (ESI) m/z: 489 (M+H⁺).

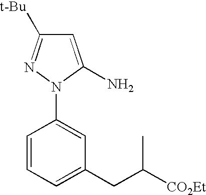

### Example NNN

To a suspension of 2-(3-bromo-phenyl)-5-t-butyl-2H-pyrazol-3-ylamine (5.8 g, 20 mmol), Pd(OAc)₂ (450 mg, 2 mmol), PPh₃ (1.0 g, 4 m-mol), and K₂CO₃ (5.5 g, 40 mmol) in DMF (50 mL) was added 2-methyl-acrylic acid ethyl ester (2.8 g, 25 mmol) at RT under N₂. The mixture was stirred at 80° C. overnight, concentrated under reduced pressure, and purified by column chromatography to afford (E)-3-[3-(5-amino-3-t-butyl-1H-pyrazol-1-yl)phenyl]-2-methylacrylic acid (3.2 g). MS (ESI) m/z: 328 (M+H⁺)

A mixture of (E)-3-(3-(3-t-butyl-5-amino-1H-pyrazol-1-yl)phenyl)-2-methylacrylic acid ethyl ester (3.0 g, 9.14 mmol) and Pd/C (0.3 g) in methanol (50 mL) was stirred at RT under 40 psi of H₂ for 2 h. The reaction mixture was filtered and the filtrate was concentrated to afford ethyl 3-[3-(5-amino-3-t-butyl-1H-pyrazol-1-yl)phenyl]-2-methylpropanoate (2.5 g, 83% yield). MS (ESI) m/z: 330 (M+H⁺).

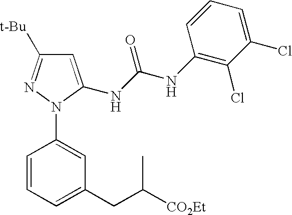

### Example 322

Using the same procedure as for Example 201, Example NNN (200 mg, 0.61 mmol) and 1,2-dichloro-3-isocyanatobenzene (187 mg, 1.0 mmol) were combined to yield 180 ethyl 3-(3-{3-t-butyl-5-[3-(2,3-dichlorophenyl)ureido]-1H-pyrazol-1-yl}phenyl)-2-methylpropanoate (180 mg, 57% yield). MS (ESI) m/z: 517 (M+H⁺).

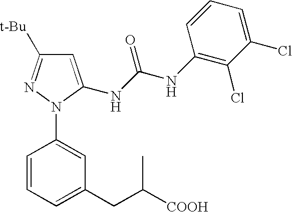

### Example 323

Using the same procedure as for Example 203, Example 322 (100 mg, 0.19 mmol) was saponified to afford 3-(3-{3-t-butyl-5-[3-(2,3-dichlorophenyl)ureido]-1H-pyrazol-1-yl}-1-phenyl)-2-methylpropanoic acid (60 mg, 65% yield). ¹H-NMR (DMSO-d₆): δ 9.20 (s, 1H), 8.72 (s, 1H), 8.03 (m, 1H), 7.43-7.19 (m, 6H), 6.34 (s, 1H), 2.95 (m, 1H), 2.69-2.62 (m, 2H), 1.24 (s, 9H), 1.01 (d, J=6.3 Hz, 3H); MS (ESI) m/z: 489 (M+H).

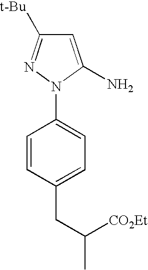

### Example OOO

To a mixture of 4-bromo-phenylhydrazine hydrochloride (22.2 g, 0.10 mol) and 4,4-dimethyl-3-oxo-pentanenitrile (13.7 g, 0.11 mol) in ethanol (100 mL) was added conc. HCl (10 mL). The resulting mixture was heated to reflux for 3 h. After removal of the solvent, the residue was purified by column chromatography to yield 2-(4-bromophenyl)-5-t-butyl-2H-pyrazol-3-ylamine hydrochloride (30 g). ¹H-NMR (400 MHz, DMSO-d₆): δ 7.76 (d, J=8.8 Hz, 2H), 7.55 (d, J=8.8 Hz, 2H), 5.63 (s, 1H), 1.27 (s, 9H); MS (ESI) m/z: 294 (M+H).

To a solution of 2-(4-bromophenyl)-5-t-butyl-2H-pyrazol-3-ylamine (3.94 g, 10 mmoL), Pd(OAc)₂ (224 mg, 10% moL), PPh₃ (520 mg, 20% mol.) and K₂CO₃ (3.28 g, 40 mmoL) in DMF (10 mL) was added 2-methyl-acrylic acid ethyl ester (1.88 mL, 15 mmoL) under N₂. The resulting mixture was stirred at 90° C. for 12 h. After removal of the solvent, the residue was extracted with EtOAc (3×150 mL). The combined organic extracts were washed with brine, dried (Na₂SO₄), filtered, concentrated and purified via column chromatography to yieldo of ethyl 3-[4-(5-amino-3-t-butyl-pyrazol-1-yl)-phenyl]-2-methyl-acrylate (900 mg).

A mixture of ethyl 3-[4-(5-amino-3-t-butyl-pyrazol-1-yl)-phenyl]-2-methyl-acrylate (900 mg, 2.7 mmol) and Pd/C (0.1 g) in EtOH (20 mL) was stirred at RT under 40 psi of H₂ for 2 h, and then filtered through celite. The filtrate was concentrated to afford ethyl 3-[4-(5-amino-3-t-butyl-1H-pyrazol-1-yl)phenyl]-2-methylpropanoate (850 mg), which was used for the next reaction without further purification.

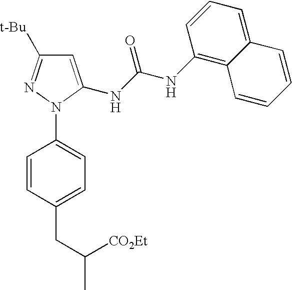

### Example 324

Using the same procedure as for Example 201, Example OOO (100 mg, 0.30 mmol) and 1-naphthyl isocyanate (70 mg, 0.41 mmol) were combined to afford ethyl 3-(4-{3-t-butyl-5-[3-(naphthalen-1-yl)ureido]-1H-pyrazol-1-yl}phenyl)-2-methylpropanoate (75 mg, 50% yield). ¹H-NMR (CD₃OD): δ 7.87 (m, 2H), 7.69 (m, 2H), 7.43-7.51 (m, 5H), 7.38 (d, J=8.0 Hz, 2H), 6.43 (s, 1H), 4.08 (m, 2H), 3.05 (m, 1H), 2.80 (m, 2H), 1.33 (s, 9H), 1.18 (d, J=8.0 Hz, 3H), 1.19 (t, J=8.0 Hz, 3H); MS (ESI) m/z: 499 (M+H).

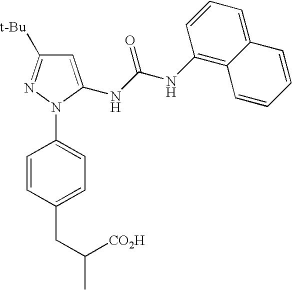

### Example 325

Using the same procedure as for Example 203, Example 324 (30 mg, 0.06 mmol) was saponified to afford 3-(4-{3-t-butyl-5-[3-(naphthalen-1-yl)ureido]-1H-pyrazol-1-yl}phenyl)-2-methylpropanoic acid (15 mg, 53% yield). ¹H NMR (DMSO-d₆): δ 9.15 (s, 1H), 8.95 (s, 1H), 8.05 (d, J=7.2 Hz, 1H), 7.89 (d, J=7.2 Hz, 1H), 7.61 (d, J=8.0 Hz, 1H), 7.41-7.55 (m, 5H), 7.32-7.34 (d, J=8.0 Hz, 2H), 6.36 (s, 1H), 2.96 (m, 1H), 2.66 (m, 2H), 1.23 (s, 9H), 1.06 (d, J=6.4 Hz, 3H); MS (ESI) m/z: 471 (M+H⁺).

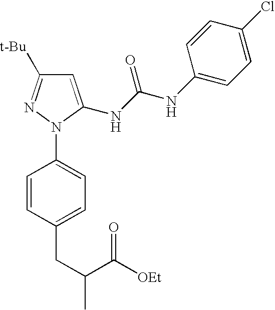

### Example 326

Using the same procedure as for Example 201, Example OOO (100 mg, 0.30 mmol) and 1-chloro-4-isocyanato-benzene (69 mg, 0.45 mmol) were combined to afford ethyl 3-(4-{3-t-butyl-5-[3-(4-chlorophenyl)ureido]-1H-pyrazol-1-yl}phenyl)-2-methylpropanoate (90 mg, 62% yield). ¹H-NMR (400 MHz, CD₃OD): δ 7.34-7.41 (m, 6H), 7.25 (d, J=8.8 Hz, 2H), 6.40 (s, 1H), 4.05-4.08 (m, 2H), 3.03 (m, 1H), 2.80 (m, 2H), 1.28 (s, 9H), 1.19 (t, J=8.0 Hz, 3H), 1.17 (d, J=6.4 Hz, 3H); MS (ESI) m/z: 483 (M+H⁺).

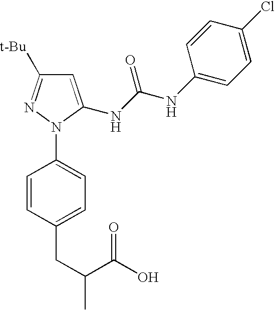

### Example 327

Using the same procedure as for Example 203, Example 326 (40 mg, 0.08 mmol) was saponified to afford 3-(4-{3-t-butyl-5-[3-(4-chlorophenyl)ureido]-1H-pyrazol-1-yl}phenyl)-2-methylpropanoic acid (18 mg, 50% yield). ¹H-NMR (400 MHz, DMSO-d₆): δ 7.37-7.43 (m, 4H), 7.24-7.30 (m, 4H), 6.28 (s, 1H), 2.95 (m, 2H), 2.64 (m, 1H), 1.24 (s, 9H), 1.15 (d, J=7.6 Hz, 3H). MS (ESI) m/e: 455 (M+H⁺).

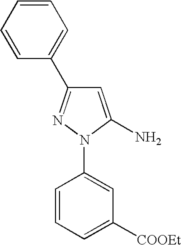

### Example PPP

To a solution of m-amino benzoic acid ethyl ester (200 g, 1.46 mmol) in concentrated HCl (200 mL) was added an aqueous solution (250 mL) of NaNO₂ (102 g, 1.46 mmol) at 0° C. and the reaction mixture was stirred for 1 h. A solution of SnCl₂.2H₂O (662 g, 2.92 mmol) in concentrated HCl (2 L) was then added at 0° C. The reaction solution was stirred for 2 h at RT. The precipitate was filtered and washed with ethanol and ether to yield ethyl 3-hydrazinobenzoate, which was used for the next reaction without further purification.

To a mixture of 3-hydrazino-benzoic acid ethyl ester (4.5 g, 25.0 mmol) and commercially available 3-oxo-3-phenyl-propionitrile (5.5 g, 37.5 mmol) in ethanol (50 mL) was added conc. HCl (5 mL). The resulting mixture was heated to reflux for 3 h. After removal of the solvent, the residue was washed with Et₂O to afford ethyl 3-(5-amino-3-phenyl-1H-pyrazol-1-yl)benzoate (7 g), which was used in the next reaction without further purification.

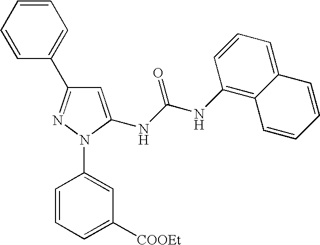

### Example 328

Using the same procedure as for Example 201, Example PPP (1.54 g, 5.0 mmol) and 1-isocyanato-naphthalene (1.0 g, 6.0 mmol) were combined to afford ethyl 3-[5-(3-naphthalen-1-yl-ureido)-3-phenyl-pyrazol-1-yl]benzoate (970 mg, 41% yield).

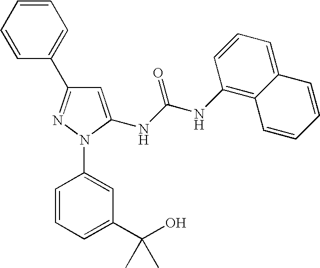

### Example 329

To a solution of Example 328 (100 mg, 0.21 mmol) in fresh THF (10 mL) was added dropwise a solution of MeMgBr (0.7 mmol, 1M in THF) at 0° C. in ice-water bath. The resulting mixture was stirred for 1 h, and then warmed to RT for 2 h. The reaction mixture was quenched with saturated NH₄Cl (10 mL) and extracted with CH₂Cl₂ (3×50 mL). The combined organic extracts were washed with saturated NaHCO₃ and brine, then dried (Na₂SO₄), filtered, concentrated and purified via preparative HPLC to afford 1-{1-[3-(2-hydroxypropan-2-yl)phenyl]-3-phenyl-1H-pyrazol-5-yl}-3-(naphthalen-1-yl)urea (85 mg, 88% yield) ¹H-NMR (300 MHz, CD₃OD): δ 7.85-7.82 (m, 4H), 7.77 (m, 1H), 7.73-7.62 (m, 3H), 7.56 (m, 1H), 7.50-7.39 (m, 6H), 7.35 (m, 1H), 6.91 (s, 1H), 1.58 (s, 6H).

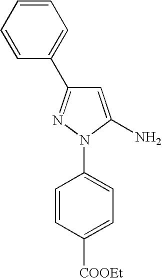

### Example QQQ

A solution of 4-aminobenzoic acid ethyl ester (200 g, 1.46 mmol) in concentrated HCl (200 mL) was added an aqueous solution (250 mL) of NaNO₂ (102 g, 1.46 mmol) at 0° C. and the reaction mixture was stirred for 1 h. A solution of SnCl₂.2H₂O (662 g, 2.92 mmol) in concentrated HCl (2 L) was then added at 0° C. The reaction solution was stirred for 2 h at RT. The precipitate was filtered and washed with ethanol and ether to yield 4-hydrazinobenzoic acid ethyl ester, which was used in the next reaction without further purification.

To a mixture of 4-hydrazinobenzoic acid ethyl ester (4.5 g, 25 mmol) and commercially available 3-oxo-3-phenylpropionitrile (5.5 g, 37.5 mmol) in ethanol (50 mL) was added conc. HCl (5 mL). The resulting mixture was heated to reflux for 3 h. After removal of the solvent, the residue was washed with Et₂O to afford ethyl 4-(5-amino-3-phenyl-1H-pyrazol-1-yl)benzoate (7.4 g), which was used in the next reaction without further purification.

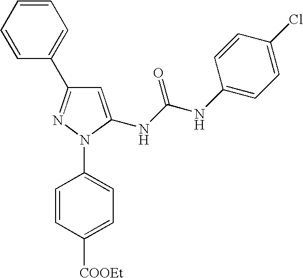

### Example 330

Using the same procedure as for Example 201, Example QQQ (1.54 g, 5.0 mmol) and 1-chloro-4-isocyanatobenzene (0.92 g, 6.0 mol) were transformed to afford ethyl 4-{5-[3-(4-chlorophenyl)ureido]-3-phenyl-1H-pyrazol-1-yl}benzoate (1.2 g, 52% yield).

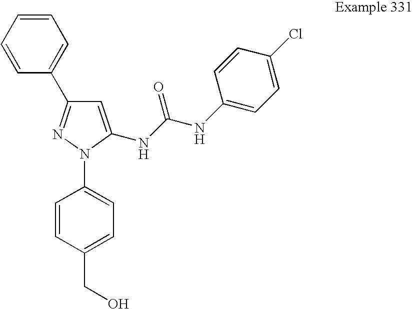

### Example 331

Using the same procedure as for Example 200, Example 330 (100 mg, 0.21 mmol) was reduced to afford 1-(4-chlorophenyl)-3-{1-[4-(hydroxymethyl)phenyl]-3-phenyl-1H-pyrazol-5-yl}-urea (70 mg, 80% yield). ¹H-NMR (300 MHz, CD₃OD): δ 9.16 (s, 1H), 8.53 (s, 1H), 7.81 (d, J=7.2 Hz, 2H), 7.54 (d, J=8.4 Hz, 2H), 7.49-7.40 (m, 4H), 7.38-7.28 (m, 3H), 6.89 (s, 1H), 5.30 (t, J=5.6 Hz, 1H), 4.56 (d, J=5.6 Hz, 2H).

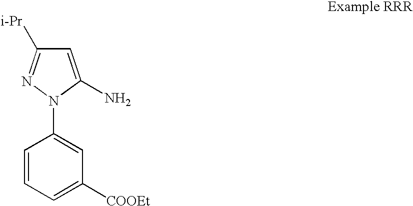

### Example RRR

To a suspension of NaH (60%, 6.0 g, 0.15 mol) in THF (100 mL) was added dropwise isobutyric acid ethyl ester (11.6 g, 0.1 mol) and anhydrous acetonitrile (50 g, 0.12 mol) in THF (100 mL) at 80° C. The resulting mixture was refluxed overnight, then cooled to RT. After removal of the volatiles in vacuo, the residue was diluted in EtOAc and aqueous 10% HCL. The combined organic extracts were dried (Na₂SO₄), filtered, concentrated to yield 4-methyl-3-oxopentanenitrile (8.5 g), which was used for the next step reaction without further purification.

To a mixture of ethyl 3-hydrazino-benzoate (from Example OO, 3 g, 16.6 mmol) and 4-methyl-3-oxopentanenitrile (2.7 g, 24.9 mmol) in ethanol (50 mL) was added conc. HCl (5 mL). The resulting mixture was heated to reflux for 3 h. After removal of the solvent, the residue was washed with Et₂O to afford ethyl 3-(5-amino-3-isopropyl-1H-pyrazol-1-yl)-benzoate (4 g), which was used in the next reaction without further purification.

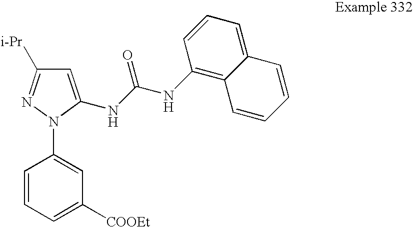

### Example 332

Using the same procedure as for Example 201, Example RRR (1.37 g, 5.0 mmol) and 1-isocyanato-naphthalene (1.0 g, 60 mol) were combined to afford ethyl 3-(3-isopropyl-5-[3-(naphthalen-1-yl)ureido]-1H-pyrazol-1-yl)benzoate (1.02 g, 46% yield).

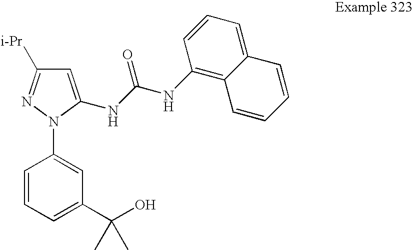

### Example 323

Using the same procedure as for Example 329, Example 332 (100 mg, 0.23 mmol) was transformed to afford 1-{1-[3-(2-hydroxypropan-2-yl)phenyl]-3-isopropyl-1H-pyrazol-5-yl}-3-(naphthalen-1-yl)urea (80 mg, 81% yield). ¹H-NMR (300 MHz, DMSO-d₆): δ 9.00 (s, 1H), 8.78 (s, 1H), 7.95 (m, 1H), 7.90-7.87 (m, 2H), 7.63-7.60 (m, 2H), 7.54-7.34 (m, 6H), 6.33 (s, 1H), 2.88 (m, 1H), 1.43 (s, 6H), 1.21 (d, J=6.9 Hz, 3H).

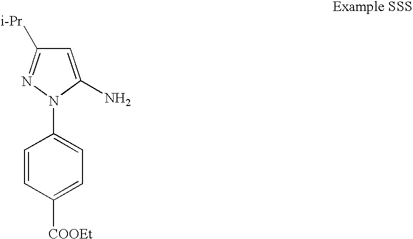

### Example SSS

To a mixture of 4-hydrazino-benzoic acid ethyl ester (from Example PP, 3 g, 16.6 mmol) and 4-methyl-3-oxo-pentanenitrile (from Example QQ, 2.7 g, 27.9 mmol) in ethanol (50 mL) was added conc. HCl (5 mL). The resulting mixture was heated to reflux for 3 h. After removal of the solvent, the residue was washed with Et₂O to afford ethyl 4-(5-amino-3-isopropyl-1H-pyrazol-1-yl)benzoate (4 g, 88% yield), which was used to the next reaction without further purification.

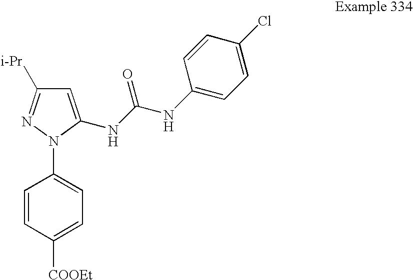

### Example 334

Using the same procedure as for Example 201, Example SSS (1.37 g, 5.0 mmol) and 1-chloro-4 isocyanatobenzene (0.9 g, 60 mol) were combined to afford ethyl 4-{5-[3-(4-chlorophenyl)ureido]-3-isopropyl-1H-pyrazol-1-yl}benzoate (1.3 g, 61% yield).

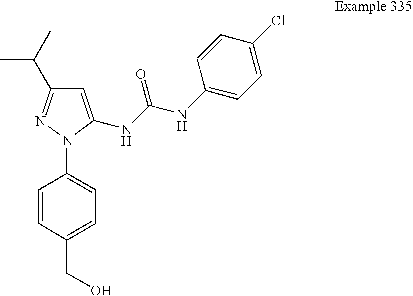

### Example 335

Using the same procedure as for Example 200, Example 334 (100 mg, 0.23 mmol) was reduced to afford 1-(4-chlorophenyl)-3-{1-[4-(hydroxymethyl)phenyl]-3-isopropyl-1H-pyrazol-5-yl}-urea (80 mg, 91% yield). ¹H-NMR (400 MHz, DMSO-d₆): δ 9.15 (br s, 1H), 8.70 (br s, 1H), 7.46-7.36 (m, 6H), 7.26 (d, J=8.8 Hz, 2H), 6.25 (s, 1H), 5.28 (t, J=6.0 Hz, 1H), 4.52 (d, J=5.2 Hz, 2H), 2.85 (m, 1H), 1.20 (d, J=6.8 Hz, 6H).

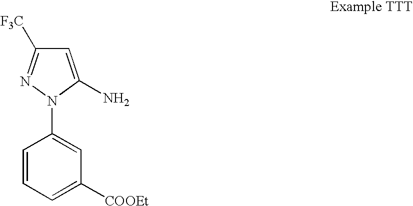

### Example TTT

To a mixture of 3-hydrazino-benzoic acid ethyl ester (from Example PPP, 3 g, 16.6 mmol) and commercially available 4,4,4-trifluoro-3-oxo-butyronitrile (3.4 g, 24.9 mmol) in ethanol (50 mL) was added conc. HCl (5 mL). The resulting mixture was heated to reflux for 3 h. After removal of the solvent, the residue was washed with Et₂O to afford ethyl 3-[5-amino-3-(trifluoromethyl)-1H-pyrazol-1-yl]benzoate (4.5 g, 91% yield), which was used to the next reaction without further purification.

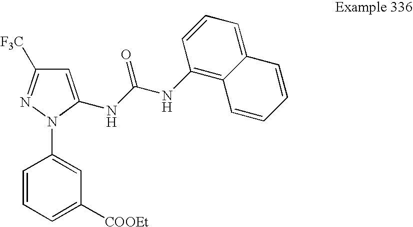

### Example 336

Using the same procedure as for Example 201, Example TTT (1.5 g, 5.0 mmol) and 1-isocyanato-naphthalene (1.0 g, 6.0 mmol) were combined to afford ethyl 3-{5-[3-(naphthalen-1-yl)ureido]-(3-(trifluoromethyl)-1H-pyrazol-1-yl}benzoate (0.9 g, 38% yield).

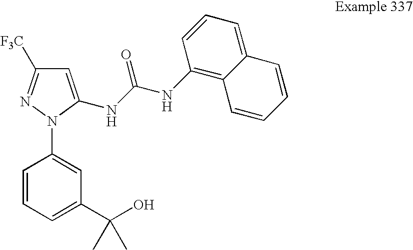

### Example 337

Using the same procedure as for Example 329, Example 336 (100 mg, 021 mmol) was reduced to afford 1-{1-[3-(2-hydroxypropan-2-yl)phenyl]-(3-(trifluoromethyl)-1H-pyrazol-5-yl}-3-(naphthalen-1-yl)urea (50 mg, 52% yield). ¹H-NMR (300 MHz, DMSO-d₆): δ 9.13 (s, 1H), 9.09 (s, 1H), 7.97-7.87 (m, 3H), 7.69-7.63 (m, 4H), 7.58-7.43 (m, 5H), 6.89 (s, 1H), 1.46 (s, 6H).

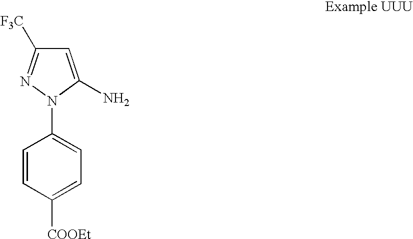

### Example UUU

To a mixture of 4-hydrazino-benzoic acid ethyl ester (From Example PP, 3.0 g, 16.6 mmol) and commercially available 4,4,4-trifluoro-3-oxobutyronitrile (3.4 g, 24.9 mmol) in ethanol (50 mL) was added conc. HCl (5 mL). The resulting mixture was heated to reflux for 3 h. After removal of the solvent, the residue was washed with Et₂O to afford ethyl 4-[5-amino-3-(trifluoromethyl)-1H-pyrazol-1-yl]benzoate (4.5 g, 91% yield), which was used to the next reaction without further purification.

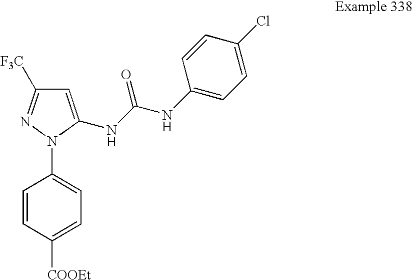

### Example 338

Using the same procedure as for Example 201, Example UUU (1.45 g, 5.0 mmol) and 1-chloro-4-isocyanatobenzene (0.9 g, 6.0 mol) were combined to afford ethyl 4-{5-[3-(4 chlorophenyl)ureido]-3-(trifluoromethyl)-1H-pyrazol-1-yl}benzoate (0.85 g, 38% yield).

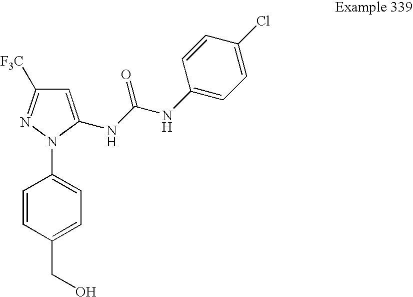

### Example 339

Using the same procedure as for Example 200, Example 338 (100 mg, 0.22 mmol) was reduced to afford 1-(4-chlorophenyl)-3-{3-(trifluoromethyl)-1-[4-(hydroxyl-methyl)phenyl]-1H-pyrazol-5-yl}urea (80 mg, 89% yield) ¹H-NMR (400 MHz, DMSO-d₆): δ 9.65 (s, 1H), 9.09 (s, 1H), 7.54 (d, J=8.4 Hz, 2H), 7.48 (d, J=8.4 Hz, 2H), 7.41 (d, J=8.8 Hz, 2H), 7.28 (d, J=8.8 Hz, 2H), 6.81 (s, 1H), 5.36 (t, J=6.0 Hz, 1H), 4.56 (d, J=5.6 Hz, 2H).

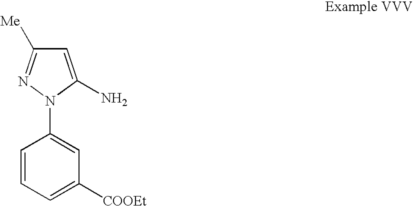

### Example VVV

To a suspension of NaH (60%, 12.0 g, 0.3 mol) in THF (200 mL) was added dropwise acetic acid ethyl ester (17 g, 0.2 mol) and anhydrous acetonitrile (100 g, 0.24 mol) in THF (200 mL) at 80° C. The resulting mixture was refluxed overnight, and then cooled to RT. After removal of the volatiles in vacuo, the residue was diluted in EtOAc and aqueous 10% HCL. The combined organic extracts were washed with saturated NaHCO₃ and brine, then dried (MgSO₄), filtered, concentrated to yield 3-oxobutyronitrile (10 g), which was used for the next step reaction without further purification.

To a mixture of 3-hydrazino-benzoic acid ethyl ester (from Example OO, 3.0 g, 16.6 mmol) and 3-oxo-butyronitrile (2.1 g, 24.9 mmol) in ethanol (50 mL) was added conc. HCl (5 mL). The resulting mixture was heated to reflux for 3 h. After removal of the solvent, the residue was washed with Et₂O to afford ethyl 3-(5-amino-3-methyl-1H-pyrazol-1-yl)benzoate (4 g), which was used to the next reaction without further purification.

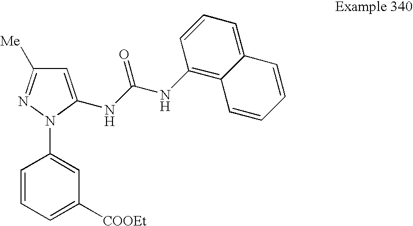

### Example 340

Using the same procedure as for Example 201, Example VVV (490 mg, 2.0 mmol) and 1-isocyanato-naphthalene (0.5 g, 3.0 mmol) were combined to afford ethyl 3-{3-methyl-5-[3-(naphthalen-1-yl)ureido]-1H-pyrazol-1-yl}benzoate (400 mg, 48% yield).

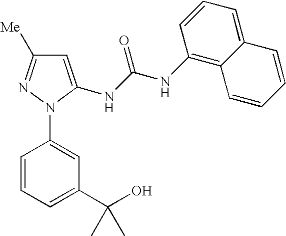

### Example 341

Using the same procedure as for Example 329, Example 340 (100 mg, 0.24 mmol) was reduced to afford 1-{1-[3-(2-hydroxypropan-2-yl)phenyl]-3-methyl-1H-pyrazol-5-yl}-3-(naphthalen-1-yl)urea (80 mg, 83% yield). ¹H-NMR (300 MHz, CDCl₃): δ 8.61 (br s, 1 H), 8.34 (br s, 1H), 7.91-7.78 (m, 2H), 7.67-7.61 (m, 3H), 7.45-7.35 (m, 4. H), 7.22 (m, 1H), 7.06 (m, 1H), 6.59 (s, 1H), 2.67 (s, 3H), 1.45 (s, 6H).

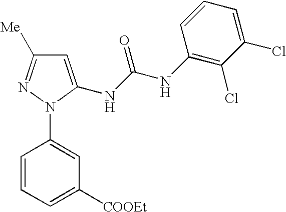

### Example 342

Using the same procedure as for Example 201, Example VVV (490 g, 2.0 mmol) and 1,2-dichloro-3-isocyanatobenzene (448 mg, 3.0 mmol) were combined to afford ethyl 3-{5-[3-(2,3-dichlorophenyl)ureido]-3-methyl-1H-pyrazol-1-yl}benzoate (310 mg, 36% yield).

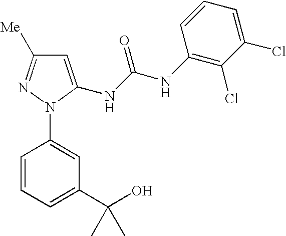

### Example 343

Using the same procedure as for Example 329, Example 342 (100 mg, 0.23 mmol) was reduced to afford 1-(2,3-dichlorophenyl)-3-{1-[3-(2-hydroxypropan-2-yl)phenyl]-3-methyl-1H-pyrazol-5-yl}urea (90 mg, 93% yield). ¹H-NMR (300 MHz, CDCl₃): δ 8.15 (br s, 1H), 8.06 (m, 1H), 7.95 (s, 1H), 7.69 (s, 1H), 7.42 (d, J=5.7 Hz, 2H), 7.30 (m, 1H), 7.19-7.17 (m, 2H), 6.51 (s, 1H), 2.36 (s, 3H), 1.56 (s, 6H).

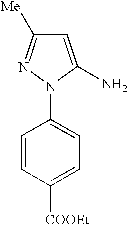

### Example WWW

To a mixture of 4-hydrazinobenzoic acid ethyl ester (3.0 g, 16.6 mmol) and 3-oxo-butyronitrile (2.1 g, 25 mmol) in ethanol (50 mL) was added conc. HCl (5 mL). The resulting mixture was heated to reflux for 3 h. After removal of the solvent, the residue was washed with Et₂O to afford ethyl 4-(5-amino-3-methyl-1H-pyrazol-1-yl)benzoate (4 g, 98% yield), which was used to the next reaction without further purification.

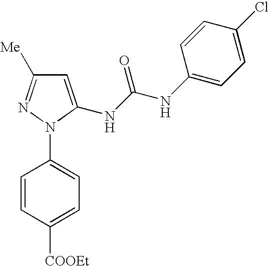

### Example 344

Using the same procedure as for Example 201, Example WWW (1.25 g, 5.0 mmol) and 1-chloro-4-isocyanatobenzene (0.9 g, 6.0 mmol) were combined to afford ethyl 4-{5-[3-(4-chlorophenyl)ureido]-3-methyl-1H-pyrazol-1-yl}benzoate (1.2 g, 60% yield).

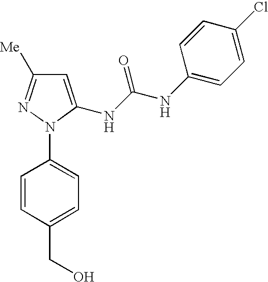

### Example 345

Using the same procedure as for Example 200, Example 344 (100 mg, 0.25 mmol) was reduced to afford 1-(4-chlorophenyl)-3-{1-[4-(hydroxymethyl)phenyl]-3-methyl-1H-pyrazol-5-yl}urea (85 mg, 96% yield). ¹H-NMR (300 MHz, DMSO-d₆): δ 9.83 (s, 1H), 8.90 (s, 1H), 7.47-7.37 (m, 6H), 7.28-7.25 (m, 2H), 6.19 (s, 1H), 5.31 (t, J=6.0 Hz, 1H), 4.51 (d, J=5.7 Hz, 2H), 2.16 (s, 3H).

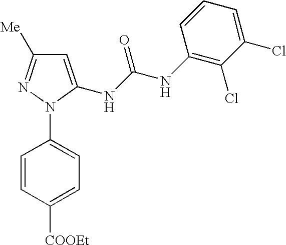

### Example 346

Using the same procedure as for Example 201, Example WWW (1.25 g, 5.0 mmol) and 1,2-dichloro-3-isocyanatobenzene (1.12 g, 6.0 mol) were combined to afford 870 mg of ethyl 4-{5-[3-(2,3-dichlorophenyl)ureido]-3-methyl-1H-pyrazol-1-yl}benzoate (870 mg, 40% yield).

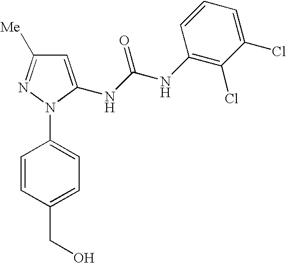

### Example 347

Using the same procedure as for Example 200, Example 346 (100 mg, 0.23 mmol) was reduced to afford 76 mg of 1-(2,3-dichlorophenyl)-3-{1-[4-(hydroxymethyl)phenyl]-3-methyl-1H pyrazol-5-yl}urea (76 mg, 85% yield). ¹H-NMR (300 MHz, CD₃OD): δ 9.59 (s, 1H), 8.95 (s, 1H), 7.99 (m, 1H), 7.48-7.40 (m, 4H), 7.28-7.26 (m, 2H), 6.22 (s, 1H), 5.35 (m, 1H), 4.52 (d, J=4.8 Hz, 2H), 2.17 (s, 3H).

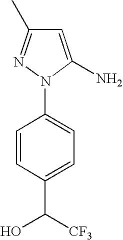

### Example XXX

To a solution of 4-nitrobenzaldehyde (15.1 g, 0.1 mol) in THF (100 mL) was added trimethyltrifluoromethylsilane (21.3 g, 0.15 mol) and Bu₄NF (500 mg) at 0° C. under N₂ atmosphere. The resulting mixture was stirred at 0° C. for 1 h and was then warmed to RT. After stirring at RT for 2 h, the reaction mixture was treated of 3.0 N HCl (100 mL). The mixture was then stirred for 1 h, then extracted with CH₂Cl₂ (3×150 mL). The combined organic extracts were washed with saturated NaHCO₃ and brine, then dried (Na₂SO₄), filtered, concentrated and purified via by column chromatography to afford of the desired product 1-(4-nitrophenyl)-2,2,2-trifluoroethanol (17.2 g). ¹H NMR (DMSO-d₆): δ 8.25 (d, J=8.8 Hz, 2H), 7.76 (d, J=8.4 Hz, 2H), 7.15 (d, J=5.6 Hz, 1H), 5.41 (m, 1H).

To a solution of 1-(4-nitrophenyl)-2,2,2-trifluoroethanol (16.0 g, 72 mmol) in methanol (50 mL) was added Pd/C (1.6 g). The mixture was stirred at RT under H₂ at 40 psi for 2 h. After filtration through celite, the filtrate was concentrated to afford 1-(4-aminophenyl)-2,2,2-trifluoroethanol (12 g), which was used for the next reaction without further purification. MS (ESI) m/z: 192 (M+H⁺).

To a stirring solution of 1-(4-aminophenyl)-2,2,2-trifluoroethanol (12 g, 63 mmol) in conc. HCl (80 mL) was added dropwise aqueous NaNO₂ (4.5 g, 65 mmol) at 0° C., and stirred for 1 h. A solution of SnCl₂ (29.5 g, 0.13 mol) in conc. HCl (100 mL) was then added dropwise to the mixture, which was stirred 0° C. for 2 h, then quench with water and neutralized to pH 8. The reaction mixture was extracted with CH₂Cl₂ (3×150 mL). The combined organic extracts were washed with saturated NaHCO₃ and brine, then dried (Na₂SO₄), filtered, concentrated to yield 1-(4-hydrazinophenyl)-2,2,2-trifluoroethanol (10 g), which was used for the next reaction without further purification. MS (ESI) m/z: 207 (M+H⁺).

To a solution of 1-(4-hydrazinophenyl)-2,2,2-trifluoroethanol (1.0 g, 41 mmol) and 3-oxobutyronitrile (500 mg) in ethanol (50 mL) was added 5 mL of conc. HCl. The resulting mixture was heated to reflux for 3 h. After removal of the solvent, the residue was purified by column chromatography to afford 1-[(4-(5-amino-3-methyl-1H-pyrazol-1-yl)phenyl]-2,2,2-trifluoroethanol (1.1 g). MS (ESI) m/z: 272 (M+H⁺).

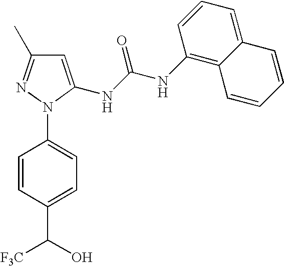

### Example 348

Using the same procedure as for Example 201, Example XXX (500 mg, 1.8 mmol) and 1-isocyanatonaphthalene (338 mg, 2.0 mol) were combined to afford 1-{3-methyl-1-[4-(2,2,2-trifluoro-1-hydroxyethyl)phenyl]-1H-pyrazol-5-yl}-3-(naphthalen-1-yl)urea (100 mg, 13% yield). ¹H NMR (DMSO-d₆): δ 9.01 (s, 1H), 8.85 (s, 1H), 7.97 (d, J=7.2 Hz, 1H), 7.91-7.85 (m, 2H), 7.64-7.40 (m, 8H), 6.30 (5, 1H), 5.24 (m, 1H), 2.17 (s, 3H); MS (EST) m/z: 441 (M+H⁺).

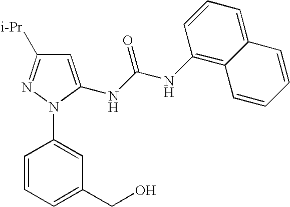

### Example 349

Using the same procedure as for Example 200, Example 332 (1.5 g, 3.4 mmol) was reduced to afford 1-{1-[3-(hydroxymethyl)phenyl]-3-isopropyl-1H-pyrazol-5-yl}-3-(naphthalen-1-yl)-urea (1.2 g, 88% yield), which was used for the next reaction without further purifications. MS (ESI) m/z: 401 (M+H⁺).

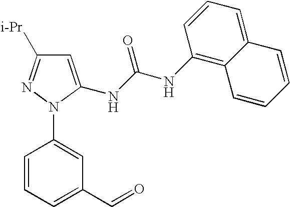

### Example 350

To a solution of Example 349 (1.0 g, 2.5 mmol) in CH₂Cl₂ (50 mL) was added MnO₂ (1.0 g) at RT. The mixture was stirred overnight then filtered. The filtrate was concentrated to afford 1-[1-(3-formylphenyl)-3-isopropyl-1H-pyrazol-5-yl]-3-(naphthalen-1-yl)urea (700 mg, 70% yield), which was used for the next reaction without further purifications. MS (ESI) m/z: 399 (M+H⁺).

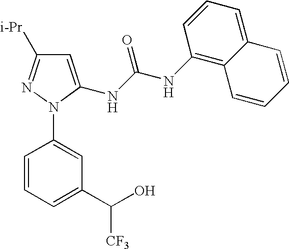

### Example 351

To a solution of Example 350 (500 mg, 1.25 mmol) in THF (50 mL) was added trimethyltrifluoromethylsilane (213 mg, 1.5 mmol) and TBAF (20 mg) at 0° C. The mixture was stirred at RT overnight before quenched with 2.0 N HCl (150 mL). The mixture was then extracted with CH₂Cl₂ (3×150 mL). The combined organic extracts were washed with saturated NaHCO₃ and brine, then dried (Na₂SO₄), filtered, concentrated purified via preparative HPLC to afford 1-(3-isopropyl-1-[3-(2,2,2-trifluoro-1-hydroxyethyl)phenyl]-1H-pyrazol-5-yl)-3-(naphthalen-1-yl)urea (80 mg, 14% yield). ¹H NMR (DMSO-d₆): δ 9.02 (s, 1H), 8.84 (s, 1H), 7.98 (d, J=7.2 Hz, 1H), 7.90-7.85 (m, 2H), 7.64-7.40 (m, 8H), 6.35 (s, 1H), 5.24 (m, 1H), 2.84 (m, 1H), 1.22 (s, 3H), 1.19 (s, 3H); MS (ESI) m/z 469 (M+H⁺).

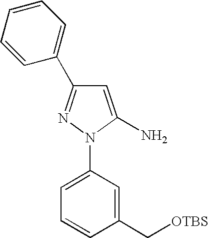

### Example YYY

Using the same procedure as for Example KK, benzoylacetonitrile (300 mg, 2.1 mmol) and 1-Boc-1-(3-carbinol)phenylhydrazine (From Example KK, 500 mg, 2.1 mmol) were combined, and then protected with TBSCl as described to afford 1-{3-[(t-butyldimethylsilyloxy)methyl]phenyl}-3-phenyl-1H-pyrazol-5-amine as a brown oil (650 mg, 82% yield). MS (ESI) m/z: 380 (M+H⁺).

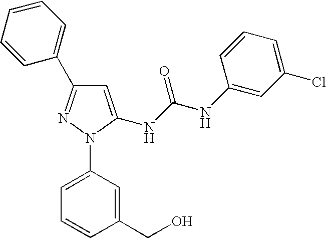

### Example 352

Using the same procedure as for Example 303, Example YYY (120 mg, 0.32 mmol) and 3-chlorophenyl isocyanate (49 mg, 0.32 mmol) were combined to yield 1-{3-phenyl-1-[3-(hydroxymethyl)phenyl]-1H-pyrazol-5-yl}-3-(3-chlorophenyl)urea as a white powder (19 mg, 47% yield). ¹H-NMR (DMSO-d₆): δ 9.32 (s, 1H), 8.66 (s, 1H), 7.86 (m, 1H), 7.70 (t, J=1.6 Hz, 1H), 7.58 (br s, 1H), 7.2-7.55 (m, 7H), 7.04 (m, 1H), 6.95 (s, 1H), 4.51 (s, 2H); MS (EI) m/z: 419 (M+H⁺).

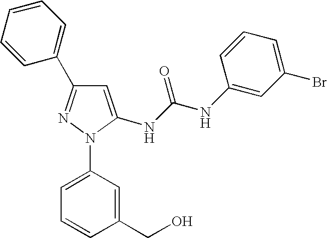

### Example 353

Using the same procedure as for Example 303, Example YYY (120 mg, 0.32 mmol) and 3-bromophenyl isocyanate (63 mg, 0.32 mmol) were combined to yield 1-(3-bromophenyl)-3-{1-[3-(hydroxymethyl)phenyl]-3-phenyl-1H-pyrazol-5-yl}urea as a white powder (33 mg, 75% yield). ¹H-NMR (DMSO-d₆): δ 9.26 (s, 1H), 8.63 (s, 1H), 7.86 (m, 2H), 7.57 (s, 1H), 7.2-7.55 (m, 6H), 7.17 (dt, J=1.8, and 7.4 Hz, 1H), 6.94 (s, 1H), 5.19 (br s, 1H), 4.61 (s, 2H); MS (EI) m/z: 463 and 465 (M⁺ and M+2H⁺).

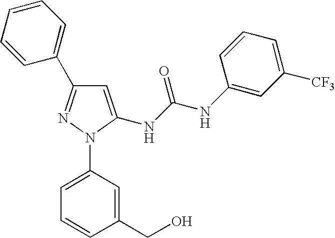

### Example 354

Using the same procedure as for Example 303, Example YYY (120 mg, 0.32 mmol) and 3-(trifluoromethyl)phenyl isocyanate (59 mg, 0.32 mmol) were combined to yield 1-{3-phenyl-1-[3-(hydroxymethyl)phenyl]-1H-pyrazol-5-yl}-3-(3-trifluoromethylphenyl)urea as a white powder (30 mg, 77% yield). ¹H-NMR (DMSO-d₆): δ 9.47 (br s, 1H), 9.03 (br s, 1H), 8.00 (s, 1H), 7.86 (d, J=8.4 Hz, 2H), 7.64 (s, 1H), 7.1-7.6 (m, 9H), 6.92 (s, 1H), 5.49 (t, J=5.6 Hz, 1H), 4.59 (d, J=5.6 Hz, 2H); MS (EI) m/z: 453 (M+H).

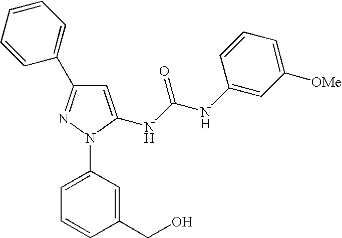

### Example 355

Using the same procedure as for Example 303, Example YYY (120 mg, 0.32 mmol) and 3-methoxyphenyl isocyanate (50 mg, 0.32 mmol) were combined to yield 1-{3-phenyl-1-[3-(hydroxymethyl)phenyl]-1H-pyrazol-5-yl}-3-(3-methoxyphenyl)urea as a white powder (22 mg, 50% yield). ¹H-NMR (DMSO-d₆): δ 9.07 (s, 1H), 9.03 (br s, 1H), 8.52 (s, 1H), 7.85 (m, 2H), 7.56 (s, 1H), 7.1-7.55 (m, 7H), 6.94 (s, 1H), 6.91 (dd, J=1.2, and 8.1 Hz, 1H), 6.56 (dd, J=1.8, and 7.5 Hz, 1H), 5.31 (br s, 1H), 4.61 (br s, 2H), 3.72 (s, 3H); MS (EI) m/z: 415 (M+H⁺).

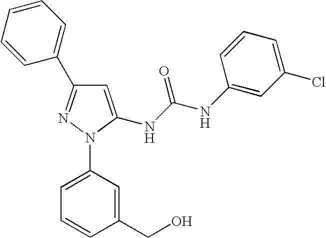

### Example 356

Using the same procedure as for Example 303, Example YYY (120 mg, 0.32 mmol) and 2,3-dichlorophenyl isocyanate (59 mg, 0.32 mmol) were combined to yield 1-{3-phenyl-1-[3-(hydroxymethyl)phenyl]-1H-pyrazol-5-yl}-3-(2,3-dichlorophenyl)urea as a white powder (29 mg, 71% yield). ¹H-NMR (DMSO-d₆): δ 9.37 (s, 1H), 8.85 (s, 1H), 8.08 (m, 1H), 7.85 (m, 2H), 7.58 (s, 1H), 7.3-7.55 (m, 8H), 6.95 (s, 1H), 5.38 (t, J=5.7 Hz, 1H), 4.61 (d, J=5.7 Hz, 2H); MS (EI) m/z: 453 (M+H⁺).

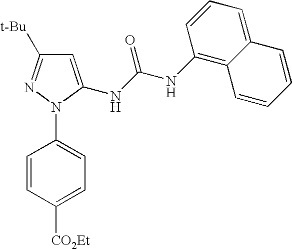

### Example 357

Using the same procedure as for Example 201, Example MMM (4.86 g, 15 mmol) and 1-isocyanato-naphthalene (3.38 g, 20 mmol) were combined to afford ethyl 4-{3-t-butyl-5-[3-(naphthalen-1-yl)ureido]-1H-pyrazol-1-yl}benzoate (1.45 g, 22% yield), which was used without further purification.

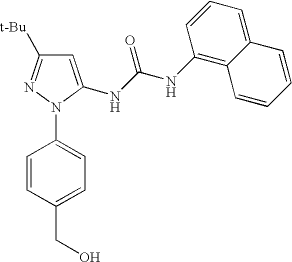

### Example 358

Using the same procedure as for Example 200, Example 357 (1.8 g, 3.21 mmol) was reduced to afford 1-{3-t-butyl-1-[4-(hydroxymethyl)phenyl]-1H-pyrazol-5-yl}-3-(naphthalen-1-yl)urea (1.2 g, 90% yield), which was used without further purification.

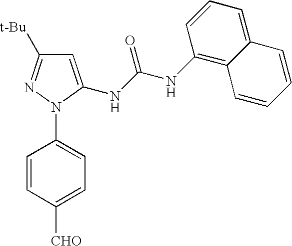

### Example 359

To a solution of Example 358 (200 mg, 0.48 mmol) in fresh CH₂Cl₂ was added powder activated MnO₂ (1.0 g, 12 mmol) and the resulting mixture was stirred at RT overnight. After filteration through, the filtrate was concentrated to afford 1-[3-t-butyl-1-(4-formylphenyl)-1H-pyrazol-5-yl]-3-(naphthalen-1-yl)urea (180 mg, 91% yield), which was used for the next step without further purification.

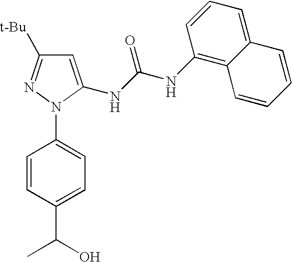

### Example 360

To a solution of Example 359 (100 mg, 0.24 mmol) in fresh THF (40 mL) was added dropwise a solution of methylmagnesium bromide (0.86 mL, 1.4 mol/L in toluene/THF) at 0° C. under N₂. After stirring for 1 h, the resulting mixture was allowed to rise to RT and stirred for 1 h. The reaction mixture was quenched by addition of aqueous solution of HCl (1 mol/L, 50 mL) and extracted with EtOAc (3×50 mL). The combined organic layers were washed with brine, dried (Na₂SO₄), filter, concentrated and purified via column chromatography to afford 1-{3-t-butyl 1-[4-(1-hydroxyethyl)phenyl]-1H-pyrazol-5-yl}-3-(naphthalen-1-yl)urea (55 mg, 54% yield). ¹H-NMR (300 MHz, DMSO-d₆): δ 9.05 (s, 1H), 8.80 (s, 1H), 7.98-7.42 (m, 11H), 6.39 (s, 1H), 4.79 (q, J=6.6 Hz, 1H), 1.36 (d, J=6.6 Hz, 3H), 1.27 (s, 9H).

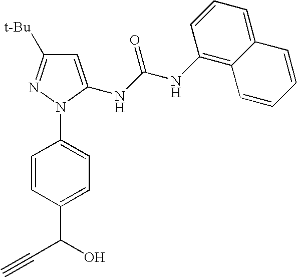

### Example 361

To a solution of Example 359 (100 mg, 0.24 mmol) in fresh THF (40 mL) was added dropwise a solution of ethynylmagnesium bromide (2.42 mL, 0.5 mol/L in toluene/THF) at 0° C. under N₂. After stirred for 1 h, the resulting mixture was allowed to rise to RT and stirred for 1 h. The reaction mixture was quenched by addition of aqueous solution of HCl (1 mol/L, 50 mL) and extracted with EtOAc (3×100 mL). The combined organic layers were washed with brine, dried (Na₂SO₄), filter, concentrated and purified via column chromatography to afford 1-{3-t-butyl-1-[4-(1-hydroxyprop-2-ynyl)phenyl]-1H-pyrazol-5-yl}-3-(naphthalen-1-yl)urea (40 mg, 39% yield). ¹H-NMR (300 MHz, DMSO-d₆): δ 9.05 (br s, 1H), 8.83 (br s, 1H), 8.00 (d, J=7.8 Hz, 1H), 7.90 (d, J=9 Hz, 2H), 7.65-7.42 (m, 8H), 6.40 (s, 1H), 5.43 (d, J=2.1 Hz, 1H), 3.53 (d, J=2.4 Hz, 1H), 1.27 (s, 9H).

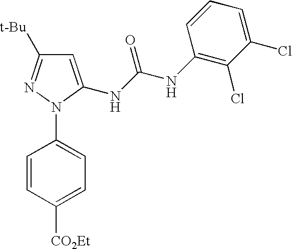

### Example 362

Using the same procedure as for Example 201, Example MMM (1 g, 3.09 mmol) and 1,2-dichloro-3-isocyanato-benzene (0.7 g, 3.71 mmol) were combined to afford ethyl 4-{3-t-butyl-5-[3-(2,3-dichlorophenyl)ureido]-1H-pyrazol-1-yl}benzoate (0.7 g, 48% yield). ¹H-NMR (300 MHz, DMSO-d₆): δ 9.20 (br s, 1H), 8.77 (br s, 1H), 8.04 (m, 1H), 7.44 (br s, 4H), 7.29-7.26 (m, 2H), 6.36 (s, 1H), 4.31 (q, J=7.2 Hz, 2H), 1.27 (s, 9H), 1.26 (t, J=7.2 Hz, 3H).

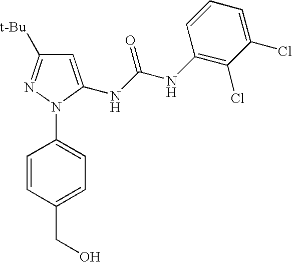

### Example 363

Using the same procedure as for Example 200, Example 362 (80 mg, 0.17 mmol) was reduced to afford 1-{3-t-butyl-1-[(hydroxymethyl)phenyl]-1H-pyrazol-5-yl}-3-(2,3-dichloro-phenyl)urea (50 mg, 68% yield). ¹H-NMR (300 MHz, DMSO-d₆): δ 9.20 (br s, 1H), 8.77 (br s, 1H), 8.04 (m, 1H) 7.45 (br s, 4H), 7.30-7.25 (m, 2H), 6.36 (s, 1H), 4.55 (s, 2H), 1.27 (s, 9H).

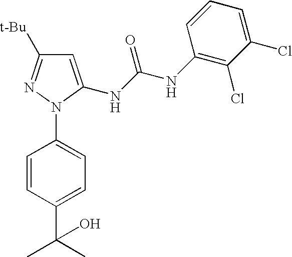

### Example 364

To a solution of Example 362 (100 mg, 0.21 mmol) in fresh THF (10 mL) was added dropwise a solution of methylmagnesium bromide (1.5 mL, 1.4 mol/L in toluene/THF) at 0° C. under N₂. After stirring for 1 h, the resulting mixture was allowed to rise to RT and stirred for 1 h. The reaction mixture was quenched by

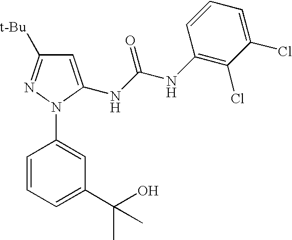

### Example 373

Using the same procedure as for Example 364, Example 371 (100 mg, 0.21 mmol) was reduced to afford 1-{3-t-butyl-1-[3-(2-hydroxypropan-2-yl)phenyl]-1H-pyrazol-5-yl}-3-(2,3-dichlorophenyl)urea (50 mg, 52% yield). ¹H-NMR (300 MHz, DMSO-d₆): δ 9.19 (br s, 1H), 8.72 (br s, 1H), 8.06 (dd, J=36.6 Hz, 1H), 7.58 (m, 1H), 7.46-7.43 (m, 2H), 7.32-7.27 (m, 3H), 6.36 (s, 1H), 1.42 (s, 6H), 1.26 (s, 9H).

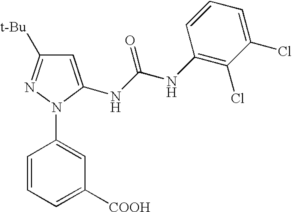

### Example 374

Using the same procedure as for Example 203, Example 374 (80 mg, 0.17 mmol) was saponified to afford 3-{3-t-butyl-5-[3-(2,3-dichlorophenyl)ureido]-1H-pyrazol-1-yl}benzoic acid (60 mg, 79% yield). ¹H-NMR (300 MHz, DMSO-d₆): δ 9.46 (br s, 1H), 8.82 (br s, 1H), 8.05 (br s, 1H), 7.98 (t, J=4.8 Hz, 1H), 7.92 (d, J=7.8 Hz, 1H), 7.80 (d, J=8.7 Hz, 1H), 7.63 (t, J=7.8 Hz, 1H), 7.27 (d, J=4.5 Hz, 2H), 6.37 (s, 1H), 1.26 (s, 9H)

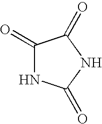

### Example ZZZ

Dry urea (3.0 g) was added to a solution of NaOMe (0.1 mol, in 50 mL of methanol) at RT, stirred for 30 min, after which diethyl oxalate (7.0 g) was slowly added. The mixture was stirred for 1 h, conc. HCl (10 mL) was added and the solution stirred for 10 min. After filtration, the residue was washed twice with a small quantity of methanol, and the combined filtrates were concentrated to yield a white solid imidazolidine-2,4,5-trione which was used without further purification. ¹H NMR (300 MHz, DMSO-d₆): δ 11.8 (s, 2H).

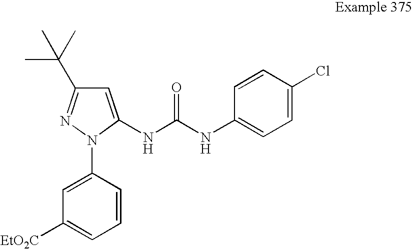

### Example 375

Using the same procedure as for Example 201, Example SS (10.7 g, 70.0 mmol) and 4-nitrophenyl 4-chlorophenylcarbamate (10 g, 34.8 mmol) were combined to yield ethyl 3-{3-t-butyl-5-[3-(4-chlorophenyl)ureido]-1H-pyrazol-1-yl}benzoate (8.0 g, 52% yield). ¹H NMR (DMSO-d₆): δ 9.11 (s, 1H), 8.47 (s, 1H), 8.06 (m, 1H), 7.93 (d, J=7.6 Hz, 1H), 7.81 (d, J=8.0 Hz, 1H), 7.65 (dd, J=8.0, 7.6 Hz, 1H), 7.43 (d, J=8.8 Hz, 2H), 7.30 (d, J=8.8 Hz, 2H), 6.34 (s, 1H), 4.30 (q, J=6.8 Hz, 2H), 1.27 (s, 9H), 1.25 (t, J=6.8 Hz, 3H); MS (ESI) m/z: 441 (M⁺+H).

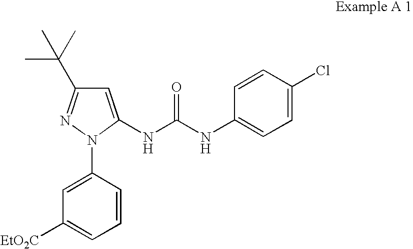

### Example A1

A solution of Example 367 (1.66 g, 4.0 mmol) and SOCl₂ (0.60 mL, 8.0 mmol) in CH₃Cl (100 mL) was refluxed for 3 h and concentrated in vacuo to yield 1-{3-t-butyl-1-[3-chloromethyl)phenyl]-1H-pyrazol-5-yl}-3-(naphthalen-1-yl)urea was obtained as white powder (1.68 g, 97% yield). ¹H NMR (DMSO-d₆): δ 9.26 (s, 1H), 9.15 (s, 1H), 8.42-7.41 (m, 11H), 6.40 (s, 1H), 4.85 (s, 2H), 1.28 (s, 9H). MS (ESI) m/z: 433 (M+H⁺).

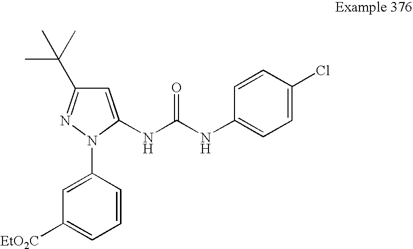

### Example 376

To a stirred solution of Example 375 (1.60 g, 3.63 mmol) in THF (200 mL) was added LiAlH powder (413 mg, 10.9 mmol) at −10° C. under N₂. The mixture was stirred for 2 h and excess LiAlH₄ was quenched by adding ice. The solution was acidified to pH=7 with dilute HCl. Solvents were slowly removed and the solid was filtered and washed with EtOAc (200+100 mL). The filtrate was concentrated to yield 1-{3-t-butyl-1-[3-hydroxymethyl)phenyl]-1H-pyrazol-5-yl}-3-(4-chlorophenyl)urea (1.40 g, 97% yield). ¹H NMR (DMSO-d₆): δ 9.11 (s, 1H), 8.47 (s, 1H), 7.47-7.27 (m, 8H), 6.35 (s, 1H), 5.30 (t, J=5.6 Hz, 1H), 4.55 (d, J=5.6 Hz, 2H), 1.26 (s, 9H); MS (ESI) m/z: 399 (M+H⁺).

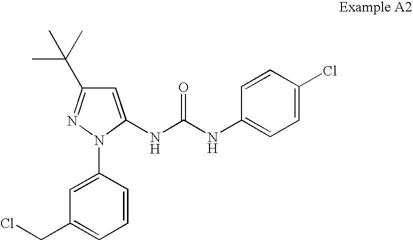

### Example A2

A solution of Example 375 (800 mg, 2.0 mmol) and SOCl₂ (0.30 mL, 4 mmol) in CH₂Cl₃ (30 mL) was refluxed gently for 3 h. The solvent was evaporated in vacuo and the residue was taken up to in CH₂Cl₂ (2×20 mL). After removal of the solvent, 1-{3-t-butyl-1-[3-(chloromethyl)phenyl]-1H-pyrazol-5-yl}-3-(4-chlorophenyl)urea (812 mg, 97% yield) was obtained as white powder. ¹H NMR (DMSO-d₆): δ 9.57 (s, 1H), 8.75 (s, 1H), 7.63 (s, 1H), 7.50-7.26 (m, 7H), 6.35 (s, 1H), 4.83 (s, 2H), 1.27 (s, 9H); MS (ESI) m/z: 417 (M+H⁺).

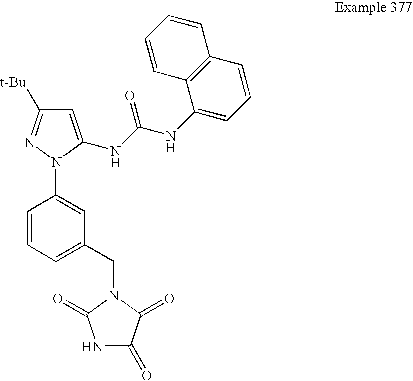

### Example 377

To a mixture of Example A1 (100 mg, 0.23 mmol), K₂CO₃ (64 mg, 0.46 mmol) and KI (10 mg) in DN (2 mL) was added Example YYY (27.0 mg, 0.23 mmol) at RT. The resulting mixture was stirred at RT overnight. The reaction solution was concentrated in vacuo, and the residue purified by column chromatography to yield 1-{3-t-butyl-1-(3-[(2,4,5-trioxoimidazolidin-1-yl)methyl]phenyl)-1H-pyrazol-5-yl}-3-(naphthalen-1-yl)urea (50 mg, 43% yield). ¹H-NMR (300 MHz, DMSO-d₆): δ 12.10 (s, 1H), 9.06 (s, 1H), 8.93 (s, 1H), 8.03 (d, J=6.0 Hz, 1H), 7.89 (d, J=6.0 Hz, 1H), 7.62-7.41 (m, 8H), 6.41 (s, 1H), 4.73 (s, 2H), 1.27 (s, 9H).

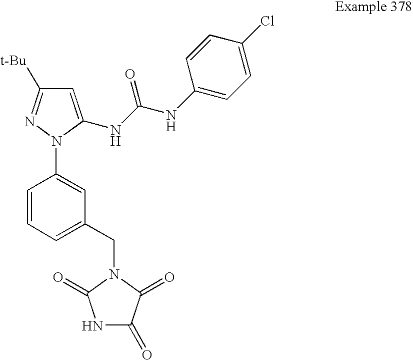

### Example 378

Using the same procedure as for Example 377, Example A2 (100 mg, 0.24 mmol), and Example YYY (29.0 mg, 0.24 mmol) were combined to afforded 1-{3-t-butyl-2-{3-[(2,4,5-trioxoimidazolidin-1-yl)methyl]phenyl}-1H-pyrazol-3-yl}-3-(4-chlorophenyl)urea (55 mg, 46% yield). ¹H-NMR (300 MHz, DMSO-d₆): δ 12.10 (s, 1H), 9.00 (s, 1H), 8.45 (s, 1H), 7.50-7.35 (m, 6H), 7.28 (d, J=8.7 Hz, 2H), 6.37 (s, 1H), 4.70 (s, 2H), 1.27 (s, 9H).

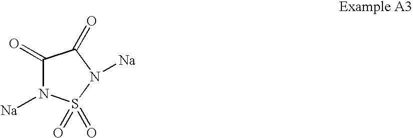

### Example A3

To a solution of NaOMe (0.15 mol, in 60 mL of methanol) was added 7.2 g of sulfamide at RT. The resulting mixture was stirred for 30 min, after which dimethyl oxalate (11.0 g) was added. The suspension mixture was heated to reflux for 16 h, cooled filtered, the precipitate washed with MeOH, and dried under vacuum to yield 1,2,5-thiadiazolidine-3,4-dione 1,1-dioxide as a disodium salt (12.2 g). ¹³C-NMR (300 MHz, D₂O): δ 173 (s, 2 C).

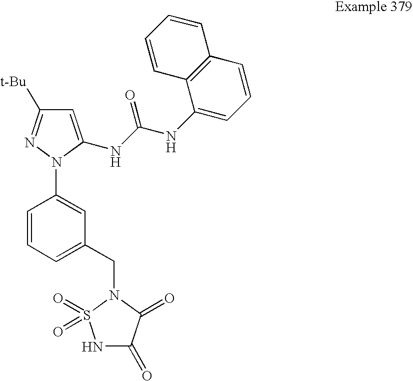

### Example 379

To a mixture of Example A31 (100 mg, 0.23 mmol) in DMF (2 mL) was added Example A3 (89.0 mg, 0.46 mmol) at RT, which was stirred overnight at RT. The reaction solution was concentrated and the residue purified via column chromatography to yield 1-(5-t-butyl-2-[3-(1,1,3,4-tetraoxo-1λ⁶-[1,2,5]thiadiazolidin-2-ylmethyl)phenyl]-2H-pyrazol-3-yl)-3-(naphthalen-1-yl)urea (35 mg, 28% yield). ¹H-NMR (300 MHz, CD₃OD): 7.83-7.92 (m, 2H), 7.64-7.69 (m, 3H), 7.40-7.57 (m, 6H), 6.47 (s, 1H), 4.90 (s, 2H), 1.28 (s, 9H).

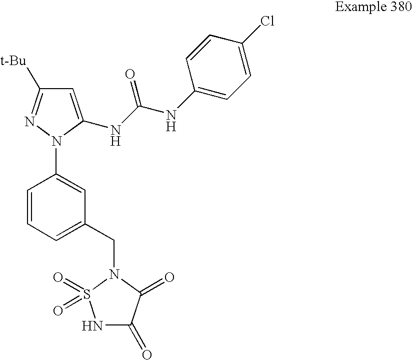

### Example 380

Using the same procedure as for Example 379, Example A32 (100 mg, 0.24 mmol) and Example A3 (91.0 mg, 0.48 mmol) were combined to yield 1-{5-t-butyl-2-[3-(1,1,3,4-tetraoxo-1λ⁶-[1,2,5]thiadiazolidin-2-ylmethyl)phenyl]-2H-pyrazol-3-yl}-3-(4-chlorophenyl)urea (40 mg, 31% yield). ¹H-NMR (300 MHz, DMSO-d₆): δ 8.96 (s, 1H), 8.45 (s, 1H), 7.53 (s, 1H), 7.25-7.46 (m, 7H), 6.35 (s, 1H), 4.69 (s, 2H), 1.25 (s, 9H).

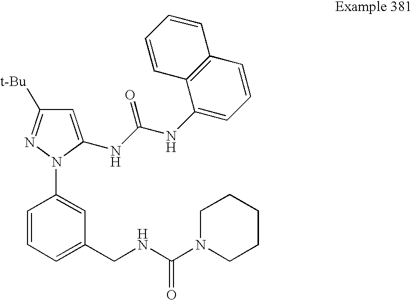

### Example 381

A mixture of Example 307 (100.0 mg, 0.28 mmol) and CDI (48.0 mg, 0.30 mmol) in DMF (2 mL) was stirred at RT for 2 h, and was followed by the addition piperidine (0.05 mL). The resulting mixture was stirred overnight, concentrated in vacuo and the residue purified by preparative HPLC to yield 1-(3-t-butyl-1-{3-[(piperidine-1-carboxamido)methyl]-phenyl}-1H-pyrazol-5-yl)-3-(naphthalen-1-yl)urea (40 mg, 27% yield). ¹H-NMR (300 MHz, DMSO-d₆): δ 9.14 (s, 1H), 8.95 (s, 1H), 8.05 (d, J=8.1 Hz, 1H), 7.88-7.94 (m, 2H), 7.62 (d, J=6.0 Hz, 2H), 7.42-7.53 (m, 6H), 7.27 (d, J=6.9 Hz, 1H), 7.06 (t, J=6.9 Hz, 1H), 6.39 (s, 1H), 4.30 (d, J=5.4 Hz, 2H), 3.25 (br s, 4H), 1.34 (br s, 4H), 1.27 (s, 9H), 1.19-1.24 (m, 2H).

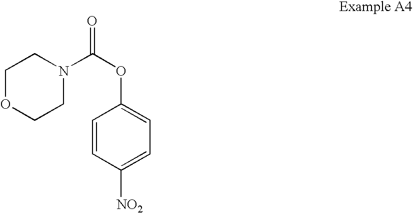

### Example A4

To a solution of 4-nitro phenyl chloroformate (0.243 g, 1.2 mmol) in THF was added morpholine (0.116 mL, 1.2 mmol) at 0° C., and the mixture stirred for 5 h and concentrated to yield 4-nitrophenyl morpholine-4-carboxylate, which was used without further purification. ¹H NMR (300 MHz, DMSO-d₆): δ 8.25 (d, J=9.0 Hz, 2H), 7.43 (d, J=9.0 Hz, 2H), 3.63-3.66 (br s, 4H), 3.59-3.62 (br s, 2H), 3.39-3.45 (br s, 2H).

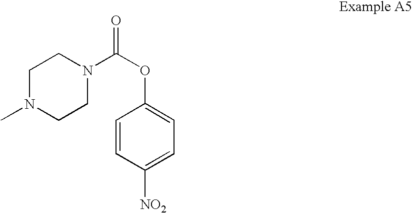

### Example A5

To a solution of 4-nitro phenyl chloroformate (0.243 g, 1.2 mmol) in THF was added 1-methyl-piperazine (0.12 mg, 1.2 mmol) at 0° C., and the mixture was stirred for 5 h and concentrated to yield 4-nitrophenyl 4-methylpiperazine-1-carboxylate, which was used without further purification. ¹H-NMR (300 MHz, DMSO-d₆): δ 8.25 (d, J=9.0 Hz, 2H), 7.42 (d, J=9.0 Hz, 2H), 3.58 (br s, 2H), 3.43 (br s, 2H), 2.47 (br. s, 4H), 2.20 (s, 3H).

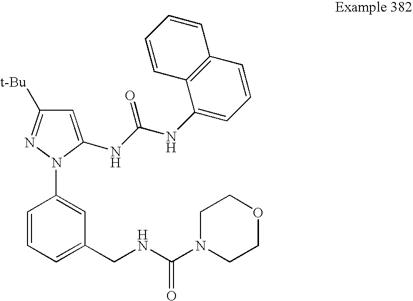

### Example 382

A solution of Example 307 (50 mg, 0.12 mmol) in DMF (1 mL) and Example CCC (30 mg, 0.12 mmol) was heated at 80° C. for overnight and purified via preparative HPLC to yield 30 mg of 1-(3-t-butyl-1-{3-[(morpholine-4-carboxamido)methyl]phenyl}-1H-pyrazol-5-yl)-3-(naphthalen-1-yl)urea (30 mg, 48% yield). ¹H-NMR (300 MHz, DMSO-d₆): δ 9.07 (s, 1H), 8.86 (s, 1H), 8.03 (d, J=8.1 Hz, 1H), 7.88-7.93 (m, 2H), 7.62 (d, J=9.0 Hz, 1H), 7.42-7.53 (m, 6H), 7.29 (d, J=9.0 Hz, 1H), 7.18 (t, J=6.0 Hz, 1H), 6.39 (s, 1H), 4.31 (d, J=5.4 Hz, 2H), 3.47 (t, J=5.1 Hz, 4H), 3.24 (t, J=5.4 Hz, 4H), 1.27 (s, 9H).

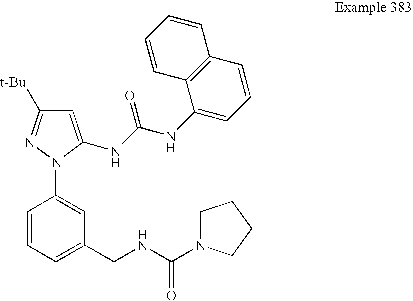

### Example 383

To a solution of pyrrolidine (0.02 mL, 0.24 mmol) in DMF (2 mL) was added NaH (10 mg, 0.24 mmol) at 0° C. The mixture was stirred for 15 min, followed by the addition of Example 307 (100 mg, 0.24 mmol) and CDI (47 mg, 0.28 mmol) in DMF (2 mL). The mixture was stirred overnight, concentrated and purified via preparative HPLC to yield 1-(3-t-butyl-1-{3-[(pyrrolidine-1-carboxamido)methyl]phenyl}-1H-pyrazol-5-yl)-3-(naphthalen-1-yl)urea (35 mg, 29% yield). ¹H-NMR (300 MHz, DMSO-d₆): δ 9.05 (s, 1H), 8.84 (s, 1H), 8.02 (d, J=8.1 Hz, 1H), 7.89-7.94 (m, 2H), 7.62 (d, J=6.9 Hz, 1H), 7.39-7.54 (m, 6H), 7.31 (d, J=7.5 Hz, 1H), 6.70 (s, 1H), 6.40 (s, 1H), 4.29 (d, J=4.8 Hz, 2H), 3.17 (t, J=6.6 Hz, 4H), 1.67 (t, J=6.6 Hz, 4H), 1.27 (s, 9H).

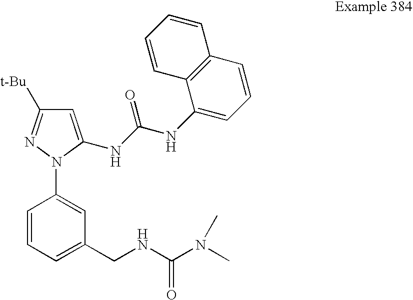

### Example 384

Using the same procedure as for Example 383, Example 307 (100 mg, 0.24 mmol) and dimethylamine (0.02 mg, 0.24 mmol) were combined to yield 35 mg of 1-{3-t-butyl-1-[3-(3,3-dimethylureidomethyl)phenyl]-1H-pyrazol-5-yl}-3-(naphthalen 1-yl)urea (35 mg, 30% yield). N,N-dimethylamino-1-carboxylic acid 3-[3-t-butyl-5-(3-naphthalen-1-yl-ureido)-pyrazol-1-yl]-benzylamide ¹H NMR (300 MHz, DMSO-d₆): δ 9.06 (s, 1H), 8.85 (s, 1H), 8.01 (d, J=9.0 Hz, 1H), 7.87-7.92 (m, 2H), 7.61 (d, J=6.0 Hz, 1H), 7.37-7.54 (m, 6H), 7.28 (d, J=9.0 Hz, 1H), 6.91 (s, 1H), 6.38 (s, 1H), 2.73 (s, 6H), 1.25 (s, 9H).

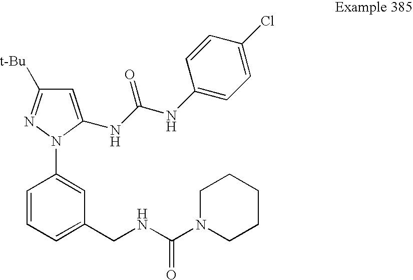

### Example 385

Using the same procedure as for Example 382, Example 287 (100 mg, 0.25 mmol) and piperidine (0.03 mL) were combined to yield 1-(3-t-butyl-1-{3-[(piperidine-1-carboxamido)methyl]phenyl}-1H-pyrazol-5-yl)-3-(4-chlorophenyl)urea (35 mg, 28% yield). ¹H-NMR (300 MHz, DMSO-d₆): δ 9.30 (s, 1H), 8.56 (s, 1H), 7.35-7.55 (m, 8H), 7.15 (t, J=6.0 Hz, 1H), 6.45 (s, 1H), 4.40-4.38 (m, 4H), 1.58-1.60 (m, 2H), 1.46-1.48 (m, 4H), 1.37 (s, 9H).

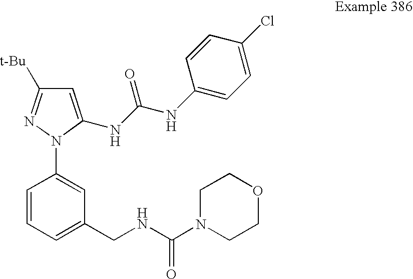

### Example 386

Using the same procedure as for Example 383, Example 287 (100.0 mg, 0.25 mmol) and morpholine (0.028 mL) were combined to yield 1-(3-t-butyl-1-{3-[(morpholine-4-carboxamido)methyl]phenyl}-1H-pyrazol-5-yl)-3-(4-chlorophenyl)urea (25 mg, 20% yield). ¹H-NMR (300 MHz, DMSO-d₆): δ 9.18 (s, 1H), 8.40 (s, 1H), 7.25-7.45 (m, 8H), 7.15 (t, J=6.0 Hz, 1H), 6.35 (s, 1H), 4.29 (d, J=5.4 Hz, 2H), 3.49 (t, J=4.8 Hz, 4H), 3.25 (t, J=4.8 Hz, 4H), 1.25 (s, 9H).

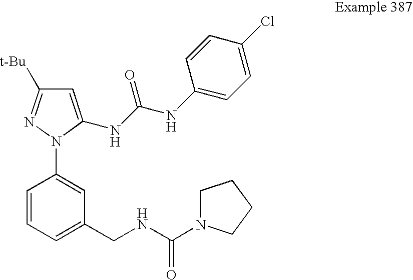

### Example 387

Using the same procedure as for Example 383, Example 287 (100.0 mg, 0.25 mmol) and pyrrolidine (0.025 mL) were combined to yield 1-(3-r-butyl-1-{3-[(pyrrolidine-1-carboxamido)methyl]phenyl}-1H-pyrazol-5-yl)-3-(4-chlorophenyl)urea (30 mg, 24% yield). ¹H-NMR (300 MHz, DMSO-d₆): δ 9.15 (s, 1H), 8.42 (s, 1H), 7.27-7.45 (m, 8H), 6.70 (t, J=6.0 Hz, 1H), 6.35 (s, 1H), 4.27 (d, J=5.4 Hz, 2H), 3.17-3.19 (m, 4H), 1.72-1.74 (m, 4H), 1.25 (s, 9H).

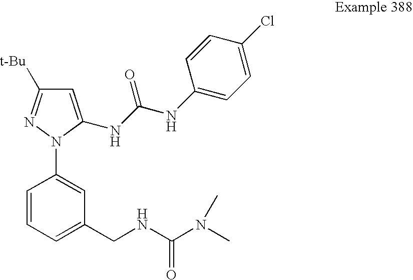

### Example 388

Using the same procedure as for Example 383, Example 287 (100 mg, 0.25 mmol) and dimethylamine (25 mg) were combined to yield 1-{3-t-butyl-1-[3-(3,3-dimethylureidomethyl)phenyl]-1H-pyrazol-5-yl}-3-(4-chlorophenyl)urea (18 mg, 15% yield). ¹H-NMR (300 MHz, DMSO-d₆): δ 9.17 (s, 1H), 8.44 (s, 1H), 7.27-7.43 (m, 8H), 6.80 (t, J=6.0 Hz, 1H), 6.34 (s, 1H), 4.26 (d, J=5.4 Hz, 2H), 2.76 (s, 6H), 1.26 (s, 9H).

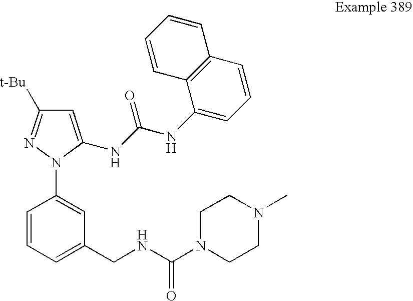

### Example 389

Using the same procedure as for Example 302, Example 307 (50 mg, 0.12 mmol) and Example A5 (32 mg, 0.12 mmol) were combined to yield 1-{3-t-butyl-1-(3-[(1-methylpiperazine-4-carboxamido)methyl]phenyl}-1H-pyrazol-5-yl)-3-(naphthalen 1-yl)urea (35 mg, 54% yield). ¹H-NMR (300 MHz, DMSO-d₆): δ 10.0 (br s, 1H), 9.10 (s, 1H), 8.89 (s, 1H), 8.00-8.02 (d, J=8.0 Hz, 1H), 7.90 (d, J=6.3 Hz, 2H), 7.63 (d, J=9.0 Hz, 1H), 7.44-7.55 (m, 6H), 7.32 (d, J=6.9 Hz, 1H), 6.39 (s, 1H), 4.32 (d, J=5.4 Hz, 2H), 4.05 (br s, 2H), 3.35 (br s, 2H), 2.80-3.10 (m, 4H), 2.74 (s, 3H), 1.27 (s, 9H).

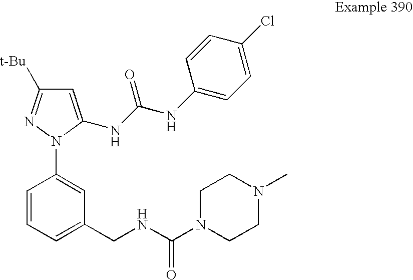

### Example 390

Using the same procedure as for Example 302, Example 287 (100.0 mg, 0.25 mmol) and 1-methyl-piperazine (0.033 mL) were combined to yield 1-{3-t-butyl-1-(3-[(1-methylpiperazine-4-carboxamido)methyl]phenyl}-1H-pyrazol-5-yl)-3-(4-chlorophenyl)urea (40 mg, 31% yield). ¹H-NMR (300 MHz, DMSO-d₆): δ 9.80 (br s, 1H), 9.22 (s, 1H), 8.48 (s, 1H), 7.27-7.43 (m, 8H), 6.34 (s, 1 M), 4.30 (d, J=5.4 Hz, 2H), 4.05-4.08 (m, 2H), 3.36-3.38 (m, 2H), 2.81-3.05 (m, 4H), 2.76 (s, 3H), 1.26 (s, 9H).

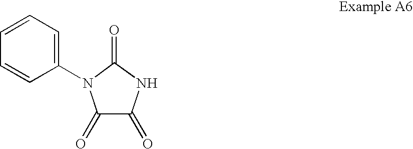

### Example A6

To a solution of aniline (2.51 g, 27 mmol) dissolved in glacial acetic acid (14 mL) and water (28 mL) was slowly added a solution of potassium cyanate (4.4 g, 54 mmol) dissolved in water (35 mL). The mixture stirred for 2 h at RT, filtered, washed with water and dried under reduced pressure to yield phenylurea as a white solid (1.85 g, 50% yield). ¹H NMR (DMSO-d₆): δ 8.47 (s, 1H), 7.38 (dd, J=8.4 Hz, 0.9 Hz, 2H), 7.2 (t, J=7.6 Hz, 2H), 6.88 (t, J=7.6 Hz, 1H), 5.81 (bs, 2H); MS (ESI) m/z: 137 (M+H⁺).

A suspension of Example FFF (0.4 g, 3 mmol) in ether (20 mL) was added oxalylchloride (0.8 g, 6 mmol) and refluxed for 3 h. Solvent was removed under reduced pressure and solid was dried to yield 1-phenylimidazolidine-2,4,5-trione (0.51 g, 89% yield), which was used without purification. ¹H NMR (DMSO-d₆): δ 7.53-7.38 (m, 5H); MS (ESI) m/z: 191 (M+H⁺).

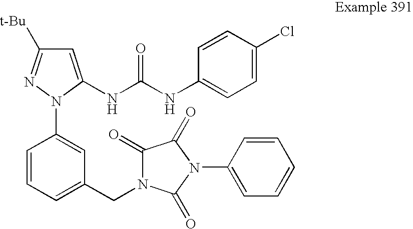

### Example 391

To a solution of triphenyl phosphine (0.23 g, 0.88 mmol) in THF (5 mL) at −20° C. were added di-t-butyl azadicarboxylate (DBAD) (0.2 g, 0.88 mmol), a solution of Example 375 (0.175 g, 0.44 mmol) in THF (5 mL) and Example A6 (0.1 g, 0.53 mmol). The resulting clear yellow solution was heated at 60° C. for 8 h, followed by the further addition of one equivalent of triphenyl phosphine and DBAD and additional heating at 60° C. overnight. One additional equivalent of triphenyl phosphine and DBAD were added and reaction mixture was heated at 60° C. for 3 h. The reaction mixture was concentrated and purified via column chromatography to yield 1-{3-t-butyl-1-(3-[(2,4,5-trioxo-3-phenylimidazolidin-1-yl)methyl]phenyl)-1H-pyrazol-5-yl}-3-(4-chlorophenyl)urea as a white solid (70 mg, 28% yield). ¹H NMR (DMSO-d₆): δ 9.02 (s, 1H), 8.45 (s, 1H), 7.53-7.28 (m, 12H), 6.39 (s, 1H), 4.87 (s, 2H), 1.28 (s, 9H); MS (ESI) m/z: 571 (M+H⁺).

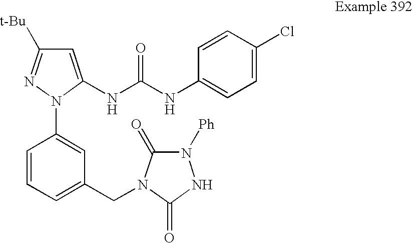

### Example 392

A mixture of 1-phenyl urazole (70 mg, 0.4 mmol), DMF (5 mL) and NaH (5 mg, 0.2 mmol) under Ar at 0° C. was stirred for 30 min. Example A2 (83 mg, 0.2 mmol) was added at 0° C., reaction mixture was warmed to RT, stirred for 8 h, quenched with water (25 mL), and extracted with EtOAc (2×25 mL). The combined organic extracts were washed with water and brine, dried (Na₂SO₄), concentrated under reduced pressure and purified by column chromatography to yield 1-(3-t-butyl-1-{3-[(3,5-dioxo-1-phenyl-1,2,4-triazolidin-4-yl)methyl]phenyl}-1H-pyrazol-5-yl)-3-(4-chlorophenyl)urea as a white solid (85 mg, 77% yield). ¹H NMR (DMSO-d₆): δ 9.06 (s, 1H), 8.49 (s, 1H), 7.48-7.29 (m, 12H), 7.24 (s, 1H), 7.1-7.08 (m, 1H), 6.36 (s, 1H), 4.64 (s, 2H), 1.28 (s, 9H); MS (ESI) m/z: 558 (M+H⁺).

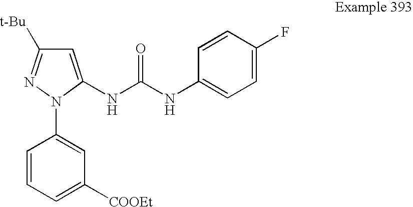

### Example 393

To a solution of Example SS (0.57 g, 2 mmol) in THF were added pyridine (0.31 g, 4 mmol) 4-fluoro phenyl isocyanate (0.27 g, 2 mmol) and reaction mixture was stirred at RT for 20 h. Then solvent was removed under reduced pressure, and the residue was solidified by stirring with hexane to yield of ethyl 3-{3-t-butyl-5-[3-(4-fluorophenyl)ureido)-1H-pyrazol-1-yl}benzoate as a white solid (0.78 g, 92% yield) ¹H NMR (DMSO-d₆): δ 9.02 (s, 1H), 8.44 (s, 1H), 8.08 (t, J=1.6 Hz, 1H), 7.95 (d, J=8.0 Hz, 1H), 7.83 (dd, J=8 Hz, 1.6 Hz, 1H), 7.67 (t, J=8 Hz, 1H), 7.42-7.39 (m, 2H), 7.09 (t, J=8.8 Hz, 2H), 6.37 (s, 1H), 4.32 (q, J=7.2 Hz, 2H), 1.30-1.28 (m, 12H); MS (ESI) m/z: 425 (M+H⁺).

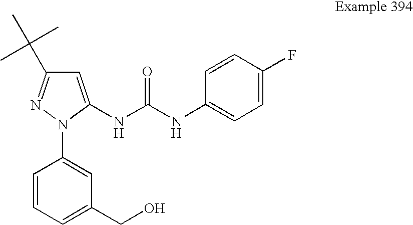

### Example 394

To a solution of Example 393 (0.78 g, 1.8 mmol) in THF (20 mL) was added LAH (5.5 mL of 1M solution in THF) at 0° C. The mixture was warmed to RT, stirred for 1 h, quenched with ice at 0° C. and concentrated under reduced pressure. The residue was acidified with 1M HCl and product was extracted with EtOAc (2×50 mL). The combined organic extracts were washed with brine, dried (Na₂SO₄) and concentrated under reduced pressure to yield 1-{3-t-butyl-1-[3-(hydroxymethyl)phenyl]-1H-pyrazol-5-yl}-3-(4-fluorophenyl)urea as a white solid (0.66 g, 94% yield) ¹H NMR (DMSO-d₆): δ 9.20 (s, 1H), 8.48 (s, 1H), 7.48-7.36 (m, 6H), 7.10 (t, J=8.8 Hz, 2H), 6.37 (s, 1H), 4.58 (s, 2H), 1.28 (s, 9H); MS (ESI) m/z: 383 (M+H⁺).

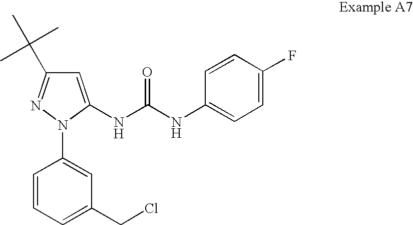

### Example A7

To a solution of Example 393 (0.45 g, 1.2 mmol) in chloroform (20 mL) was added thionyl chloride (0.28 g, 2.4 mmol) and mixture was stirred for 2 h at 65° C. Water was added and organic layer separated. The aqueous layer was extracted with CH₂Cl₂ (1×50 mL) and the combined organic extracts were washed with brine, dried (Na₂SO₄) and concentrated under reduced pressure to yield 1-{3-t-butyl-1-[3-(chloromethyl)phenyl]-1H-pyrazol-5-yl}-3-(4-fluorophenyl)urea as a solid (0.43 g, 96% yield). ¹H NMR (CDCl₃): δ 7.52 (s, 1H), 7.39-7.34 (m, 3H), 7.23-7.19 (m, 2H), 6.97-6.95 (m, 3H), 6.41 (s, 1H), 4.57 (s, 2H), 1.36 (s, 9H); MS (ESI) m/z: 401 (M+H⁺).

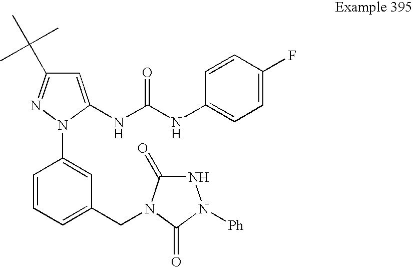

### Example 395

A solution of Example A6 (80 mg, 0.45 mmol), DMF (4 mL) and NaH (5 mg, 0.22 mmol) under Ar at 0° C. was stirred for 30 min. Example A7 (90 mg, 0.22 mmol) was added and the mixture was warmed to RT, stirred for 6 h, quenched with water (20 mL) and extracted with ethyl acetate (2×25 mL). The combined organic extracts were washed with water, brine, dried (Na₂SO₄), concentrated under reduced pressure and purified via column chromatography to yield 1-{3-t-butyl-1-(3-[(3,5-dioxo-1-phenyl-1,2,4-triazolidin-4-yl)methyl]phenyl}-1H-pyrazol-5-yl)-3-(4-fluorophenyl)urea as a white solid (65 mg, 53% yield) ¹H NMR (DMSO-d₆): δ 8.96 (s, 1H), 8.44 (s, 1H), 7.49-7.33 (m, 9H), 7.24 (s, 1H), 7.12-7.08 (m, 3H), 6.35 (s, 1H), 4.64 (s, 2H), 1.28 (s, 9H); MS (ESI) m/z: 542 (M+H⁺).

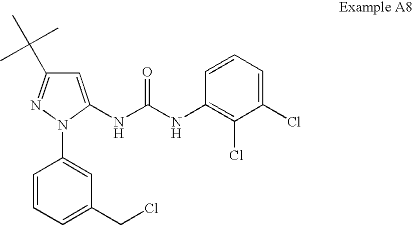

### Example A8

Using the same procedure as for Example A7, Example 371 (0.61 g, 1.4 mmol) was transformed to yield 1-(3-t-butyl-1-(3-(chloromethyl)phenyl)-1H-pyrazol-5-yl)-3-(2,3-dichlorophenyl)urea as a solid (0.6 g, 94% yield). ¹H NMR (CDCl₃): [symbol] 8.12-8.09 (m, 1H), 7.65 (s, 1H), 7.58 (s, 1H), 7.47-7.36 (m, 3H), 7.19-7.17 (m, 2H), 6.95 (br s, 1H), 6.44 (s, 1H), 4.58 (s, 2H), 1.38 (s, 9H); MS (ESI) m/z: 451 (M+H⁺).

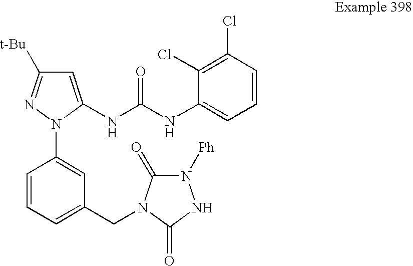

### Example 396

A solution of Example A6 (70 mg, 0.4 mmol), DMF (5 mL) and NaH (5 mg, 0.2 mmol) under Ar at 0° C. was stirred for 30 min, after which Example A8 (90 mg, 0.2 mmol) was added. The mixture was warmed to RT, stirred for 6 h, quench with water (20 ml) and extracted with EtOAc (2×). The combined organic extracts were washed with water, brine, dried (Na₂SO₄), concentrated under reduced pressure and purified via column chromatography to yield 1-(3-t-butyl-1-{3-[(3,5-dioxo-1-phenyl-1,2,4-triazolidin-4-yl)methyl]phenyl}-1H-pyrazol-5-yl)-3-(2,3-dichlorophenyl)urea as a white solid (85 mg, 72% yield). ¹H NMR (DMSO-d₆): δ 9.29 (s, 1H), 8.73 (s, 1H), 8.07 (dd, J=6.4 Hz, 3.2 Hz, 1H), 7.50-7.44 (m, 4H), 7.37-7.25 (m, 5H), 7.12-7.10 (m, 1H), 6.38 (s, 1H), 4.64 (s, 2H), 1.28 (m, 9H); MS (ESI) m/z: 592 (M+H⁺).

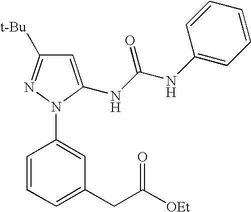

### Example 397

To a solution of Example ZZ (2 g, 6.6 mmol) and Et3N (2.2 g, 20 mmol) in THF (50 mL) was added a solution of benzene isocyanate (890 mg, 7.4 mmol) in THF (5 mL) dropwise at 0° C. under N₂ atmosphere. The mixture was warmed to RT, stirred overnight and then poured into ice aqueous solution of HCl (1 mol/L). The reaction mixture was extracted by CH₂Cl₂ (3×100 mL). The combined organic layers were washed with brine, dried (Na₂SO₄), filtered and concentrated to yield a crude solid which was purified by column chromatography to afford ethyl 2-{3-[3-t-butyl-5-(3-phenylureido)-1H-pyrazol-1-yl]phenyl}acetate (1.5 g, 54% yield). ¹H NMR (300 m/z, DMSO-d₆): δ 8.98 (s, 1H), 8.37 (s, 1H), 7.38-7.35 (m, 5H), 7.25-7.23 (m, 3H), 6.92 (t, J=7.2 Hz, 1H), 6.35 (s, 1H), 4.04 (q, J=7.2 Hz, 2H), 3.72 (s, 2H), 1.24 (s, 9H), 1.15 (t, J=7.2 Hz, 3H); MS (ESI) m/z: 421 (M+H⁺).

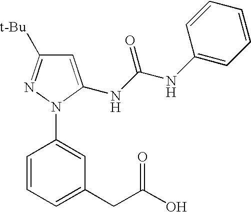

### Example 398

A mixture of Example 397 (1.4 g, 3.3 mmol) in aqueous solution LiOH (2 N, 10 mL) and THF (20 mL) was stirred at RT for 4 h. After removal of the organic solvent, the mixture was extracted with Et₂O. The aqueous solution was acidified with 2 N HCl to pH=4. The precipitate was collected, washed with brine and dried to afford 2-{3-[3-t-butyl-5-(3-phenyl-ureido)-1H-pyrazol-1-yl]phenyl}acetic acid addition of aqueous solution of HCl (5 mL, 1 M) and the mixture was extracted with EtOAc (3×). The combined organic layers were washed with brine, dried (Na₂SO₄), filter, concentrated and purified via column chromatography to afford 1-{3-t-butyl-1-[4-(2-hydroxypropan-2-yl)phenyl]-1H-pyrazol-5-yl}-3-(2,3-dichlorophenyl)urea (50 mg, 52% yield). ¹H-NMR (300 MHz, DMSO-d₆): δ 9.25 (br s, 1H), 8.79 (br s, 1H), 8.03 (m, 1H), 7.60 (d, J=8.4 Hz, 2H), 7.42 (d, J=8.4 Hz, 2H), 7.30-7.28 (m, 2H), 6.36 (s, 1H), 1.45 (s, 6H), 1.25 (s, 9H)

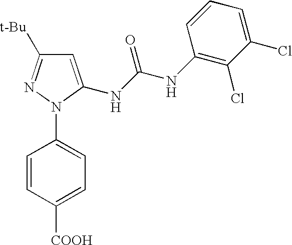

### Example 365

Using the same procedure as for Example 203, Example 362 (80 mg, 0.17 mmol) was saponified to afford 4{-3-t-butyl-5-[3-(2,3-dichlorophenyl)ureido]-1H-pyrazol-1-yl}benzoic acid (60 mg, 79% yield). ¹H-NMR (300 MHz, DMSO-d₆): δ 9.39 (br s, 1H), 8.78 (br s, 1H), 8.07-8.02 (m, 3H), 7.68 (d, J=8.4 Hz, 2H), 7.29 (d, J=7.8 Hz, 1H), 6.41 (s, 1H), 1.21 (s, 9H)

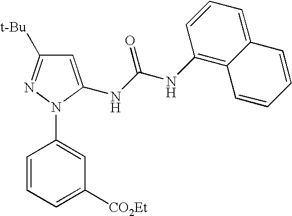

### Example 366

Using the same procedure as for Example 201, Example SS (4.86 g, 15 mmol) and 1-isocyanato-naphthalene (3.38 g, 20 mmol) were combined to afford ethyl 3-{3-t-butyl-5-[3-(naphthalen-1-yl)ureido]-1H-pyrazol-1-yl}benzoate (1.27 g, 19% yield).

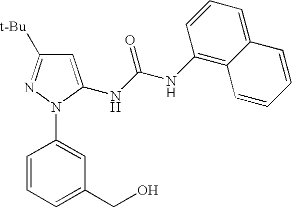

### Example367

Using the same procedure as for Example 200, Example 366 (1.46 g, 3.21 mmol) was reduced to afford 1-{3-t-butyl-1-[3-(hydroxymethyl)phenyl]-1H-pyrazol-5-yl)-3-(naphthalen-1-yl)-urea (1.1 g, 83% yield), which was used without further purifications.

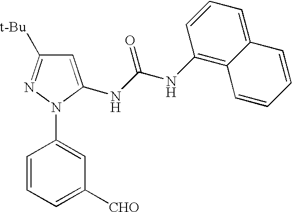

### Example 368

Using the same procedure as for Example 366, Example 367 (200 mg, 0.48 mmol) was oxidized to afford 1-[3-t-butyl-1-(3-formylphenyl)-1H-pyrazol-5-yl)-3-(naphthalen-1-yl)urea (180 mg, 91% yield), which was used without further purification.

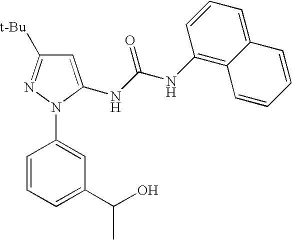

### Example 369

Using the same procedure as for Example 360, Example 368 (100 mg, 0.24 mmol) was oxidized to afford 1-{3-t-butyl-1-[3-(1-hydroxyethyl)phenyl]-1H-pyrazol-5-yl}-3-(naphthalen-1-yl)urea (35 mg, 34% yield). ¹H-NMR (300 MHz, DMSO-d₆): δ 9.03 (br s, 1H), 8.89 (br s, 1H), 8.11-7.40 (m, 11H), 6.39 (s, 1H), 3.31 (br s, 1H), 2.51 (d, J=4.8 Hz, 3H), 1.28 (s, 9H).

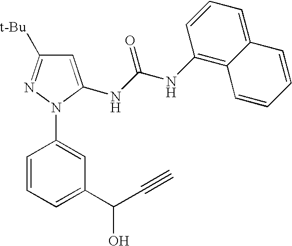

### Example 370

Using the same procedure as for Example 361, Example 368 (100 mg, 0.24 mmol) was reduced to afford 1-{3-t-butyl-1-[3-(1-hydroxyprop-2-ynyl)phenyl]-1H-pyrazol-5-yl}-3-(naphthalen-1-yl)urea (10 mg, 9.5% yield). ¹H-NMR (300 MHz, CDCl₃): δ 7.84 (d, J=8.1 Hz, 2H), 7.71 (d, J=7.8 Hz, 2H), 7.60-7.37 (m, 5H), 7.19 (m, 1H), 6.64 (s, 1H), 5.38 (br s, 1H), 2.65 (s, 1H), 2.60 (d, J=2.1 Hz, 1H), 1.36 (s, 9H).

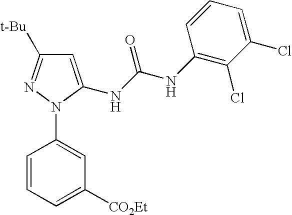

### Example 371

Using the same procedure as for Example 201, Example SS (1 g, 3.09 mmol) and 1,2-dichloro-3-isocyanato-benzene (0.7 g, 3.71 mmol) were combined to afford ethyl 3-{3-t-butyl-5-[3-(2,3-dichlorophenyl)ureido]-1H-pyrazol-1-yl}benzoate (0.6 g, 41% yield). ¹H-NMR (300 MHz, DMSO-d₆): δ 9.24 (br s, 1H), 8.70 (br s, 1H), 8.05 (t, J=1.8 Hz, 1H), 8.00 (t, J=5.1 Hz, 1H), 7.97-7.93 (m, 1H), 7.84-7.80 (m, 1H), 7.67 (t, J=8.1 Hz, 1H), 7.39 (dd, J=4.8 Hz, 2H), 6.39 (s, 1H), 4.31 (q, J=7.2 Hz, 2H), 1.27 (s, 9H), 1.26 (t, J=7.2 Hz, 3H).

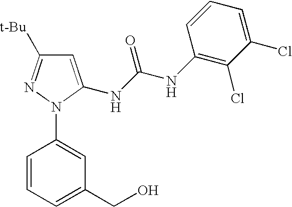

### Example 372

Using the same procedure as for Example 200, Example 371 (80 mg, 0.17 mmol) was reduced to afford 1-[3-t-butyl-1-(3-hydroxymethyl-phenyl)-1H-pyrazol-5-yl]-3-(2,3-dichlorophenyl)-urea (50 mg, 68% yield). ¹H-NMR (300 MHz, DMSO-d₆): δ 9.20 (br s, 1H), 8.75 (br s, 1H), 8.04 (dd, J=3.6 and 6 Hz 1H) 7.49-7.44 (m 2H), 7.37-7.32 (m, 2H), 7.30-7.28 (m, 2H), 6.37 (s, 1H), 4.56 (s, 2H), 1.24 (s, 9H). (0.9 g, 70% yield). ¹H NMR (300 MHz, DMSO-d₆): δ 9.07 (s, 1H), 8.40 (s, 1H), 7.39-7.35 (m, 5H), 7.25-7.23 (m, 3H), 6.93 (t, J=7.2 Hz, 1H), 6.35 (s, 1H), 3.62 (s, 2H), 1.24 (s, 9H); MS (ESI) m/z: 392 (M+H⁺).

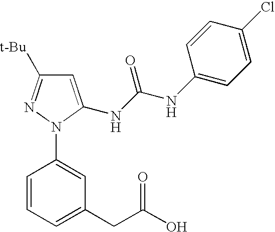

### Example 399

Using the same procedure as for Example 398, Example 320 (2.0 g, 4.4 mmol) was saponified to afford 2-(3-{3-t-butyl-5-[3-(4-chlorophenyl)ureido]-1H-pyrazol-1-yl-}phenyl)acetic acid (1.7 g, 91% yield). ¹H NMR (300 MHz, DMSO-d₆): δ 9.18 (s, 1H), 8.46 (s, 1H), 7.42-7.37 (m, 6H), 7.28-7.25 (m, 3H), 6.33 (s, 1H), 3.64 (s, 2H), 1.24 (s, 9H); MS (ESI) m/z: 427 (M+H⁺).

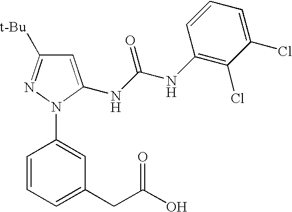

### Exampl 400

Using the same procedure as for Example 398, Example 250 (2.0 g, 4.4 mmol) was saponified i to afford 2-(3-{3-t-butyl-5-[3-(2,3-dichlorophenyl)ureido]-1H-pyrazol-1-yl}phenyl)acetic acid (1.7 g, 84% yield). ¹H NMR (300 MHz, DMSO-d₆): δ 9.26 (s, 1H), 8.76 (s, 1H), 8.03 (m, 1H), 7.48-7.35 (m, 3H), 7.27-7.25 (m, 3H), 6.36 (s, 1H), 3.64 (s, 2H), 1.24 (s, 9H); MS (ESI) m/z: 461 (M+H⁺).

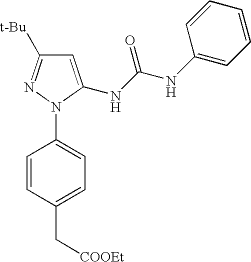

### Example 401

Using the same procedure as for Example 201, Example RR (2 g, 5.9 mmol) and benzene isocyanate (890 mg, 7.5 mmol) were combined to afford ethyl 2-{4-[3-t-butyl-5-(3-phenylureido)-1H-pyrazol-1-yl]phenyl}acetate (1.8 g, 73% yield). ¹H NMR (400 MHz, DMSO-d₆): δ 8.97 (s, 1H), 8.36 (s, 1H), 7.46-7.36 (m, 6H), 7.23 (t, J=8.1 Hz, 2H), 6.93 (t, J=7.5 Hz, 1H), 6.34 (s, 1H), 4.07 (q, J=7.2 Hz, 2H), 3.71 (s, 2H), 1.24 (s, 9H), 1.17 (t, J=7.2 Hz, 3H); MS (ESI) m/z: 421 (M+H⁺).

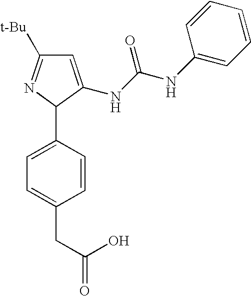

### Example 402

Using the same procedure as for Example 203, Example 402 (1.7 g, 4.0 mmol) was saponified afford 2-{4-[5-t-butyl-3-(3-phenylureido)-2H-pyrrol-2-yl]phenyl}acetic acid (1.1 g, 70% yield). ¹H NMR (300 MHz, DMSO-d₆): δ 9.04 (s, 1H), 8.42 (s, 1H), 7.45-7.36 (m, 6H), 7.23 (t, J=8.1 Hz, 2H), 6.93 (t, J=7.2 Hz, 1H), 6.34 (s, 1H), 3.62 (s, 2H), 1.25 (s, 9H); MS (ESI) m/z: 392 (M+H⁺)

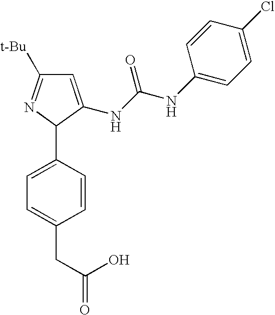

### Example 403

Using the same procedure as for Example 203, Example 317 (1.7 g, 4.0 mmol) was saponified to afford 2-(4-{5-t-butyl-3-[3-(4-chlorophenyl)ureido]-2H-pyrrol-2-yl}-phenyl)acetic acid (1.1 g, 65% yield). ¹H NMR (300 MHz, DMSO-d₆): δ 11.56 (s, 1H), 11.24 (s, 1H), 7.52-7.47 (m, 4H), 7.28 (d, J=8.4 Hz, 2H), 7.19 (d, J=8.4 Hz, 2H), 6.17 (s, 1H), 3.31 (s, 2H), 1.26 (s, 9H); MS (ESI) m/z: 426 (M+H⁺).

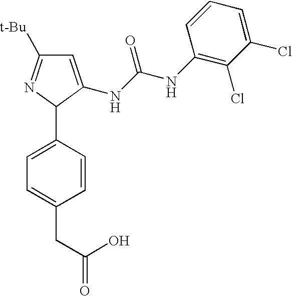

### Example 404

Using the same procedure as for Example 398, Example 321 (2.0 g, 4.1 mmol) was saponified to afford 2-(4-{5-t-butyl-3-[3-(2,3-dichlorophenyl)ureido]-2H-pyrazol-1-yl}phenyl)acetic acid (1.5 g, 80% yield). ¹H NMR (300 MHz, DMSO-d₆): δ 9.70 (s, 1H), 9.00 (s, 1H), 7.98 (m, 1H), 7.46 (d, J=8.4 Hz, 2H), 7.35 (d, J=8.4 Hz, 2H), 7.25-7.24 (m, 2H), 6.30 (s, 1H), 3.61 (s, 2H), 1.23 (s, 9H); MS (ESI) m/z: 461 (M+H⁺)

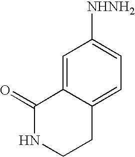

### Example A9

To a solution of phenethylamine (60.5 g, 0.5 mol) and sodium carbonate (63.6 g, 0.6 mol) in ethyl acetate/water (800 mL, 4:1) was added ethyl chloroformate dropwise (65.1 g, 0.6 mol) at 0° C. during a period of 1 h. The mixture was warmed to RT and stirred for an additional 1 h. The organic phase was separated and the aqueous layer was extracted with EtOAc. The combined organic phases were washed with water and brine, dried (Na₂SO₄), filtered and concentrated to a crude solid, which was purified by flash chromatography to afford ethyl phenethyl-carbamate (90.2 g). ¹H NMR (400 MHz, CDCl₃): δ 7.32-7.18 (m, 5H), 4.73 (br s, 1H), 4.14-4.08 (q, J=6.8 Hz, 2H), 3.44-3.43 (m, 2H), 2.83-2.79 (t, J=6.8 Hz, 2H), 1.26-1.21 (t, J=6.8 Hz, 3H).

A suspension of phenethyl-carbamic acid ethyl ester (77.2 g, 40 mmol) in polyphosphoric acid (300 mL) was heated to 140-160° C. and stirred for 2.5 h. The reaction mixture was cooled to RT, carefully poured into ice-water and stirred for 1 h. The aqueous solution was extracted with EtOAc (3×300 mL). The combined organic phases were washed with water, 5% aqueous potassium carbonate and brine, dried (Na₂SO₄), filtered and concentrated to a crude solid, which was purified by flash chromatography to afford 3,4-dihydro-2H-isoquinolin-1-one (24 g). ¹H NMR (400 MHz, DMSO-d₆): δ 7.91 (br s, 1H), 7.83 (d, J=7.5 Hz, 1H), 7.43 (t, J=7.5 Hz, 1H), 7.33-7.25 (m, 2H), 3.37-3.32 (m, 2H), 2.87 (t, J=6.6 Hz, 2H).

To an ice-salt bath cooled mixture of nitric acid and sulfonic acid (200 mL, 1:1) was added, 4-dihydro-2H-isoquinolin-1-one (15 g, 0.102 mol) dropwise over 15 min. After stirring for 2 h, the resulting mixture was poured into ice-water and stirred for 30 min. The precipitate was filtered, washed with water, dried in air to afford 7-nitro-3,4-dihydro-2H-isoquinolin-1-one (13 g). ¹H NMR (300 MHz, DMSO-d₆): δ 8.53 (d, J=2.4 Hz, 1H), 8.31 (d, J=2.4 Hz, 1H), 8.29 (d, J=2.4 Hz, 1H), 7.62 (d, J=8.4 Hz, 1H), 3.44-3.39 (m, 2H), 3.04 (t, J=6.6 Hz, 2H).

A suspension of 7-nitro-3,4-dihydro-2H-isoquinolin-1-one (11.6 g, 60 mmol) and Pd/C (1.2 g, 10%) in methanol was stirred overnight at RT under an H₂ atmosphere (40 psi). The mixture was filtered through celite and washed with methanol. The filtrate was evaporated by vacuum to afford 8.2 g of 7-amino-3,4-dihydro-2H-isoquinolin-1-one which was used without further purification.

To a suspension of 7-amino-3,4-dihydro-2H-isoquinolin-1-one (8.1 g, 50 mmol) in concentrated HCl (100 mL) was added a solution of sodium nitrite (3.45 g, 50 mmol) in water dropwise in an ice-water bath at such a rate that the reaction mixture never rose above 5-C. After stirring for 30 min, the resulting mixture was added a solution of SnCl₂ (22.5 g, 0.1 mol) in concentrated HCl (150 mL) dropwise at 0° C. in an ice-water bath. The resulting mixture was stirred for another 2 h at 0° C. The precipitate was collected by suction, washed with ether to afford 7-hydrazino-3,4-dihydro-2H-isoquinolin-1-one (8.3 g), which was used for the next reaction without further purification.

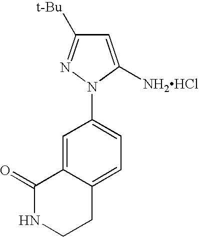

### Example A10

A mixture of Example A9 (8.0 g, 37.6 mmol) and 4,4-dimethyl-3-oxo-pentanenitrile (5.64 g, 45 mmol) in ethanol (100 mL) and concentrated HCl (10 ml) was heated to reflux overnight. After removal of the solvent, the residue was washed with ether to afford 7-(5-Amino-3-t-butyl-pyrazol-1-yl)-3,4-dihydro-2H-isoquinolin-1-one hydrochloride as a yellow solid (11.5 g, 96% yield), which was used without further purification.

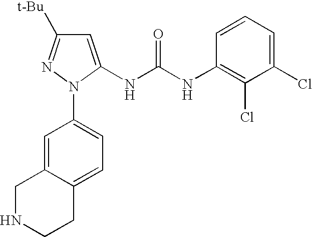

### Example 405

To a suspension of Example A10 (2.0 g, 6.2 mmol) in fresh THF (50 mL) was added a solution of Et3N (1.7 mL, 12.4 mmol) in THF (5 mL) dropwise at 0° under an N₂ atmosphere. After stirring for 30 min, 1,2-dichloro-3-isocyanato-benzene (1.42 g, 7.5 mmol) in THF (5 mL) was added dropwise via syringe to the mixture. The reaction was warmed to RT and stirred overnight. The reaction was poured onto ice cold aqueous HCl (1.0 N) and extracted with EtOAc (3×100 mL). The combined organic layers were washed with brine, dried (Na₂SO₄), filtered and concentrated to a crude solid, which was purified by flash chromatography to afford 1.2 g 1-[3-t-butyl-1-(1-oxo-1,2,3,4-tetrahydroisoquinolin-7-yl)-1H-pyrazol-5-yl]-3-(2,3-dichlorophenyl)urea (1.2 g, 41% yield). ¹H NMR (300 MHz, CDCl₃): δ 9.08 (br s, 1H), 8.34 (br s, 1H), 8.15 (br s, 1H), 8.02 (m, 1H), 7.60 (br s, 1H), 7.53 (d, J=8.1 Hz, 1H), 7.29 (d, J=8.7 Hz, 1H), 7.15-7.09 (m, 2H), 6.62 (s, 1H), 3.5 (br, 2H), 3.94 (br, 2H), 1.34 (s, 9H).

To a suspension of 1-[3-t-butyl-1-(1-oxo-1,2,3,4-tetrahydroisoquinolin-7-yl)-1H-pyrazol-5-yl]-3-(2,3-dichlorophenyl)urea (120 mg, 0.25 mmol) in fresh THF (50 mL) was added powder LAH (50 mg, 1.27 mmol) by portions in an ice-water bath. The resulting mixture was heated to reflux for 3 h, then cooled in an ice-salt bath and quenched with water and aqueous NaOH. The precipitate was filtered, washed with THF, and the combined filtrates evaporated under reduced pressure to afford 1-[3-t-butyl-1-(1,2,3,4-tetrahydroisoquinolin-7-yl)-1H-pyrazol-5-yl]-3-(2,3-dichlorophenyl)urea (80 mg, 70% yield). ¹H NMR (300 MHz, CD₃OD): δ 7.98 (t, J=4.8 Hz, 1H), 7.45-7.39 (m, 3H), 7.23 (d, J=5.1 Hz, 2H), 6.41 (s, 1H), 4.41 (s, 2H), 3.52 (t, J=6.3 Hz, 2H), 3.19 (t, J=6.3 Hz, 2H), 1.33 (s, 9H).

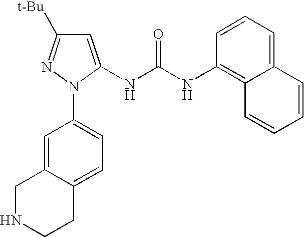

### Example 406

Using the same procedure as for Example 405, Example A10 (2.0 g, 6.2 mmol) and 1-isocyanato-naphthalene (1.27 g, 7.5 mmol) were combined to afford 1-[3-t-butyl-1-(1-oxo-1,2,3,4-tetrahydroisoquinolin-7-yl)-1H-pyrazol-5-yl]-3-(naphthalen 1-yl)urea. ¹H NMR (300 MHz, CDCl₃): δ 8.59 (br s, 1H), 8.32 (br s, 1H), 8.02 (br s, 1H), 7.85-7.04 (m, 10H), 6.62 (s, 1H), 3.42 (m, 2H), 2.83 (m, 2H), 1.34 (s, 9H)

Using the same procedure as for Example 302, 1-[3-t-butyl-1-(1-oxo-1,2,3,4-tetrahydroisoquinolin-7-yl)-1H-pyrazol-5-yl]-3-(naphthalen-1-yl)urea. (1.5 g, 3.3 mmol) was reduced to afford 1-[3-t-butyl-1-(1,2,3,4-tetrahydroisoquinolin-7-yl)-1H-pyrazol-5-yl]-3-(naphthalen-1-yl)urea (1.0 g, 69% yield). ¹H NMR (400 MHz, CDCl₃): δ 7.86-6.92 (m, 10H), 6.44 (s, 1H), 3.03 (t, J=6 Hz, 2H), 2.70 (t, J=6 Hz, 2H), 1.33 (s, 9H)

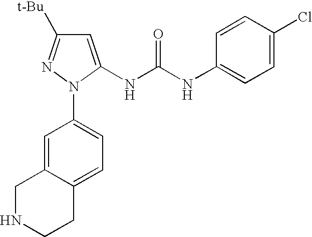

### Example 407

Using the same procedure as for Example 406, Example A00 (2.0 g, 6.2 mmol) and 1-chloro-4-isocyanatobenzene (1.15 g, 7.5 mmol) were combined to afford 1-[3-t-butyl-1-(1-oxo-1,2,3,4-tetrahydroisoquinolin-7-yl)-1H-pyrazol-5-yl]-3-(4-chlorophenyl)urea (1.5 g, 55% yield). ¹H NMR (300 MHz, CDCl₃): δ 9.03 (s, 1H), 8.77 (s, 1H), 7.90 (s, 1H), 7.54 (d, J=7.5 Hz, 1H), 7.30 (d, J=9 Hz, 3H), 7.19 (d, J=9 Hz, 2H), 6.88 (br s, 1H), 6.74 (s, 1H), 3.45 (br s, 2H), 2.88 (t, J=6 Hz, 2H), 1.37 (s, 9H)

Using the same procedure as for Example 302, 1-[3-t-butyl-1-(1-oxo-1,2,3,4-tetrahydroisoquinolin-7-yl)-1H-pyrazol-5-yl]-3-(4-chlorophenyl)urea (1.0 g, 2.3 mmol) was reduced to afford 1-[3-t-butyl-1-(1,2,3,4-tetrahydroisoquinolin-7-yl)-1H-pyrazol-5-yl]-3-(4-chlorophenyl)urea (0.8 g, 82% yield). ¹H NMR (300 MHz, DMSO-d₆): δ 9.13 (br s, 1H), 8.34 (br s, 1H), 7.41-7.12 (m, 7H), 6.31 (s, 1H), 3.88 (s, 2H), 2.95 (t, J=6 Hz, 2H), 2.70 (t, J=6 Hz, 2H), 1.24 (s, 9H).

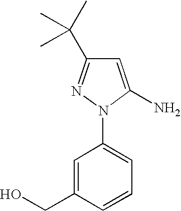

### Example A11

To a solution of Example SS (19.5 g, 68.0 mmol) in THF (200 mL) was added LiAlH₄ powder (5.30 g, 0.136 mol) at −10° C. under N₂. The mixture was stirred for 2 h at RT and excess LiAlH₄ was destroyed by slow addition of ice. The reaction mixture was acidified to pH=7 with diluted HCl, the solution concentrated under reduced pressure, and the residue was extracted with ethyl acetate. The combined organic extracts were concentrated to yield [3-(5-amino-3-t-butyl-pyrazol-1-yl)phenyl]-methanol (16.35 g, 98%) as a white powder. ¹H NMR (DMSO-d₆): 9.19 (s, 1H), 9.04 (s, 1H), 8.80 (s, 1H), 8.26-7.35 (m, 1H), 6.41 (s, 1H), 4.60 (s, 2H), 1.28 (s, 9H); MS (ESI) m/z: 415 (M+H⁺).

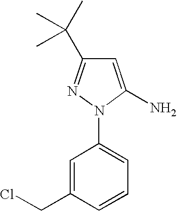

### Example A12

A solution of Example A11 (13.8 g, 56 mmol) and SOCl₂ (8.27 mL, 0.11 mol) in THF (200 ml) was refluxed for 3 h and concentrated under reduced pressure to yield 5-t-butyl-2-(3-chloromethyl-phenyl)-2H-pyrazol-3-ylamine (14.5 g, 98%) as a white powder which was used without further purification. ¹H NMR (DMSO-d₆), 67.62 (s, 1H), 7.53 (d, J=8.0 Hz, 1H), 7.43 (t, J=8.0 Hz, 1H), 7.31 (d, J=7.2 Hz, 1H), 5.38 (s, 1H), 5.23 (br s, 2H), 4.80 (s, 2H), 1.19 (s, 9H). MS (ESI) m/z: 264 (M+H⁺).

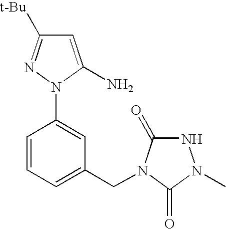

### Example A13

To a suspension of NaH (26 mg, 0.67 mmol) in DMSO (2 mL) was added powder 1-methyl-[1,2,4]triazolidine-3,5-dione (77 mg, 0.67 mmol) at RT under N₂ atmosphere. The resulting mixture was stirred for 30 min and then added to a solution of Example A12 (100 mg, 0.33 mmol) and Et₃N (1 mL) in DMSO (2 mL). After stirring for 3 h, the reaction mixture was quenched with methanol, concentrated and purified by column chromatography to afford 90 mg of 4-[3-(5-amino-3-t-butyl-pyrazol-1-yl)-benzyl]-1-methyl-[1,2,4]triazolidine-3,5-dione.

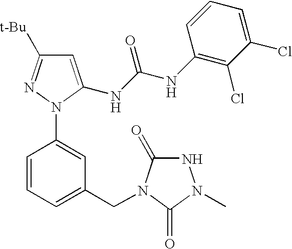

### Example 408

To a suspension of Example A13 (90 mg, 0.26 mmol) and Et3N (0.5 mL) in fresh THF (10 mL) was added a solution of 1,2-dichloro-3-isocyanato-benzene (95 mg, 0.5 mmol) in THF (2 mL) dropwise through syringe at 0° C. under N₂ atmosphere. The mixture was allowed to rise to RT and stirred overnight. The reaction mixture was quenched with ice-cold aqueous HCl (1 mol/L) and extracted with EtOAc (3×50 mL). The combined organic layers were washed with brine, dried (Na₂SO₄), filtered, concentrated and purified column chromatography to afford 80 mg of 1-{5-t-butyl-2-[3-(1-methyl-3,5-dioxo-[1,2,4)triazolidin-4-ylmethyl)-phenyl]-2H-pyrazol-3-yl}-3-(2,3-dichloro-phenyl)-urea. ¹H-NMR (DMSO-d₆), δ11.30 (s, 1H), 9.27 (s, 1H), 8.70 (s, 1H), 8.04 (m, 1H), 7.50-7.46 (m, 3H), 7.28-7.26 (m, 3H), 6.37 (s, 1H), 4.74 (s, 2H), 2.96 (s, 3H), 1.25 (s, 9H).

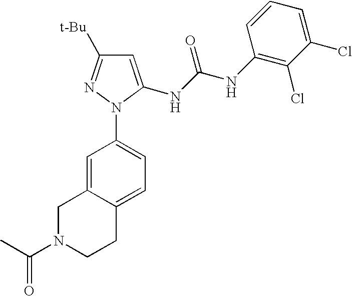

### Example 409

To a solution of Example 405 (100 mg, 0.22 mmol) and Et3N (60 μL, 0.44 mmol) in CH₂Cl₂ (2 mL) was added acetyl chloride (32 μL, 0.44 mmol) dropwise at 0° C. under N₂. The mixture was warmed to RT and stirred overnight, then poured into ice-cold 1N HCl. The reaction mixture was extracted with CH₂Cl₂ (3×20 mL), and the combined organic extracts were washed with brine, dried (Na₂SO₄), filtered, concentrated and purified via column chromatography to afford 1-[1-(2-acetyl-1,2,3,4-tetrahydroisoquinolin-7-yl)-3-t-butyl-1H-pyrazol-5-yl]-3-(2,3-dichlorophenyl)urea (55 mg, 50% yield). ¹H NMR (300 MHz, DMSO-d₆): 9.16 (m, 1H), 8.74 (s, 1H), 8.00 (s, 1H), 7.20-7.36 (m, 5H), 6.33 (s, 1H), 4.66 (s, 2H), 4.61 (s, 2H), 2.76-2.86 (m, 2H), 2.04 (s, 3H), 1.22 (s, 9H); MS (ESI) m/z: 500 (M+H⁺)

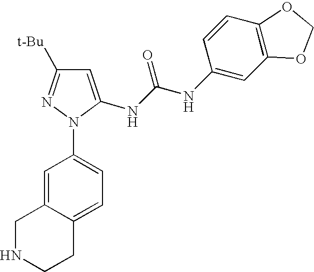

### Example 410

Using the same procedure as for Example 405, Example A10 (285 mg 1.0 mmol) and 5-Isocyanato-benzo[1,3]dioxole (163 mg, 1.0 mmol) were combined to afford 1-benzo[d][1,3)dioxol-5-yl-3-[3-t-butyl-1-(1-oxo-1,2,3,4-tetrahydroisoquinolin-7-yl)-1H-pyrazol-5-yl]urea (200 mg, 45% yield). MS (ESI) m/z: 448 (M+H⁺).

Using the same procedure as for Example 302, 1-benzo[d][1,3]dioxol-5-yl-3-[3-t-butyl-1-(1-oxo-1,2,3,4-tetrahydroisoquinolin-7-yl)-1H-pyrazol-5-yl]urea (120 mg 0.27 mmol) was reduced to afford 1-benzo[d][1,3]dioxol-5-yl-3-[3-t-butyl-1-(1,2,3,4-tetrahydroisoquinolin-7-yl)-1H-pyrazol-5-yl]urea (70 mg, 60% yield). ¹H NMR (300 MHz, DMSO-d₆): δ 9.08 (br s, 2H), 8.99 (s, 1H), 8.43 (s, 1H), 7.40-7.30 (m, 3H), 7.10 (s, 1H), 6.77 (d, J=8.4 Hz, 1H), 6.66 (d, J=8.4 Hz, 1H), 6.28 (s, 1H), 5.91 (s, 2H), 4.30 (br s, 2H), 3.35 (br s, 2H), 2.99 (t, J=6.0 Hz, 2H), 1.25 (s, 9H) MS (ESI) m/z: 434 (M+H⁺)

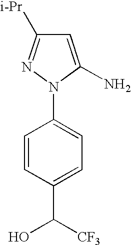

### Example A14

To a solution 4-nitro-benzaldehyde (15.1 g, 0.1 mol) in THF (100mL) was added trimethyl-trifluoromethyl-silane (21.3 g, 0.15 mol) and Bu₄NF (500 mg) at 0° C. under N₂. The resulting mixture was stirred at 0° C. for 1 h, then warmed to RT. After stirring at RT for 2 h, the reaction mixture treated with 3.0 N HCl (100 mL), then stirred for 1 h. The reaction was extracted with CH₂Cl₂ with CH₂Cl₂ (3×150 mL). The combined organic extracts were washed with brine, dried (Na₂SO₄), filtered, concentrated and purified via column chromatography to afford 17.2 g of the desired product 2,2,2-trifluoro-1-(4-nitro-phenyl)-ethanol (78%). ¹H NMR (DMSO-d₆): δ: 25 (d, J=8.8 Hz, 2H), 7.76 (d, J=8.4 Hz, 2H), 7.15 (d, J=5.6 Hz, 1H), 5.41 (m, 1H).

To a solution of 2,2,2-trifluoro-1-(4-nitro-phenyl)-ethanol (16.0 g, 72 mmol) in methanol (50 mL) was added Pd/C (1.6 g). The mixture was stirred at RT under H₂ at 40 psi for 2 h, then filtered. The filtrate was concentrated to afford 12 g of 1-(4-aminophenyl)-2,2,2-trifluoroethanol (86%), which was used for the next reaction without further purification; MS (ESI) m/z: 192 (M+H⁺)

To a solution of 1-(4-aminophenyl)-2,2,2-trifluoroethanol (12 g, 63 mmol) in conc. HCl (80 mL) was added dropwise an aqueous solution of NaNO₂ (4.3 g, 63 mmol) at 0° C., which was then stirred for 1 h. A solution of SnCl₂ (28.3 g, 0.13 mol) in con.HCl (100 mL) was added dropwise to the mixture at 0° C. The resulting mixture was stirred 0° C. for 2 h, then treated with water and neutralized to pH=8. The reaction mixture was extracted with CH₂Cl₂ (3×150 mL). The combined organic extracts were washed with brine, dried (Na₂SO₄), filtered, concentrated to yield 10 g of 2,2,2-trifluoro-1-(4-hydrazino-phenyl)-ethanol hydrochloride (65%), which was used for the next reaction without further purification; MS (ESI) m/z: 207 (M+H⁺)

A solution of 2,2,2-trifluoro-1-(4-hydrazino-phenyl)-ethanol hydrochloride (1.0 g, 4.1 mmol) and 4-methyl-3-oxo-pentanenitrile (See Example QQ, 620 mg, 5.0 mmol) in ethanol (50 mL) containing conc. HCl (5.0 mL) was heated to reflux for 3 h. After removed the solvent, the residue was purified by column chromatography to afford 1.1 g of 1-(4-(5-amino-3-isopropyl-1H-pyrazol-1-yl)phenyl)-2,2,2-trifluoroethanol (89%); MS (ESI) m/z: 300 (M+H⁺)

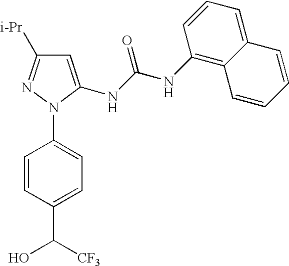

### Example 411

Using the same procedure as for Example 201, Example A14 (150 mg, 0.5 mmol) and 1-isocyanato-naphthalene (85 mg, 0.5 mmol) were combined to afford 50 mg of 1-(1-(4-(2,2,2-trifluoro-1-hydroxyethyl)phenyl)-3-isopropyl-1H-pyrazol-5-yl)-3-(naphthalen-1-yl)urea (21%). ¹H NMR (DMSO-d₆): 9.02 (s, 1H), 8.84 (s, 1H), 7.98 (d, J=7.2 Hz, 1H), 7.90-7.85 (m, 2H), 7.64-7.40 (m, 8H), 6.35 (s, 1H), 5.24 (m, 1H), 2.84 (m, 1H), 1.22 (s, 3H), 1.19 (s, 3H), MS (ESI) m/z: 469 (M+H⁺)

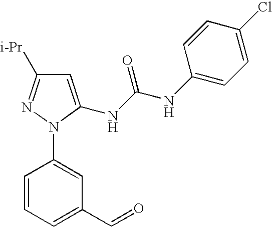

### Example 412

To a solution of Example 379 (500 mg, 1.2 mmol) in CH₂Cl₂ (200 mL) was added MnO₂ (4.3 g, 50 mmol) at RT. The mixture was stirred overnight, then filtered. The filtrate was concentrated to the crude product, which was purified via column chromatography to afford 280 mg of 1-(3-t-butyl-1-(3-formylphenyl)-1H-pyrazol-5-yl)-3-(4-chlorophenyl)urea (56%); MS (ESI) m/z: 397 (M+H⁺).

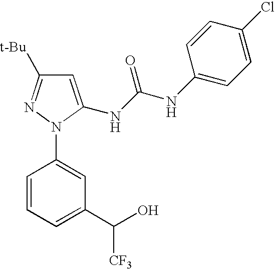

### Example 413

To a solution of Example 412 (200 mg, 0.51 mmol) in THF (20 mL) was added at 0° C. (trifluoromethyl)-trimethylsilane (85 mg, 0.60 mmol) in THF (1 mL) and then TBAF (10 mg) under N₂. The resulting mixture was stirred overnight at RT then treated with HCl (2 N, 1 mL). The reaction mixture was stirred at RT for 30 min, concentrated and the residue dissolved in CH₂Cl₂ (50 mL). The combined organic extracts were washed with saturated NaHCO₃ and brine, dried (Na₂SO₄), filtered, concentrated and purified via preparative-TLC to afford 30 mg 1-(3-t-butyl-1-(3-(2,2,2-trifluoro-1-hydroxyethyl)phenyl)-1H-pyrazol-5-yl)-3-(4-chlorophenyl)urea (13%). ¹H NMR (400 MHz, DMSO-d₆): 9.49 (s, 1H), 8.73 (s, 1H), 7.66 (s, 1H), 7.52-7.45 (m, 3H), 7.40 (d, J=8.8 Hz, 2H), 7.27 (d, J=8.8 Hz, 2H), 6.99 (s, 1H), 6.32 (s, 1H), 5.24 (m, 1H), 1.26 (s, 9H); MS (ESI) m/z: 467 (M+H⁺).

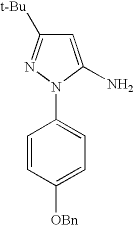

### Example A15

To a mixture of 4-nitro-phenol (10.0 g, 71.9 mmol), K₂CO₃ (19.9 g, 143.9 mmol) and KI (2.6 g, 15.8 mmol) in acetonitrile was added chloromethyl-benzene (10.0 g, 79.1 mmol) at RT. The resultant mixture was heated to reflux for 3 h. After removal of the solvent, the residue was dissolved in EtOAc. The combined organic extracts were washed with brine, dried (Na₂SO₄), filtered and concentrated to afford 14.9 g of 4-benzyloxy-nitrobenzene (90%). ¹H-NMR (400 MHz, CDCl₃): δ 8.20 (d, J=8.0 Hz, 2H), 7.43-7.37 (m, 5H), 7.03 (d, J=8.0 Hz, 2H), 5.17 (s, 2H).

A mixture of 4-benzyloxy-nitrobenzene (13.0 g, 56.5 mmol) and Re—Ni (15.0 g) in EtOH (50 mL) was stirred at RT under 30 psi of H₂. The mixture was stirred at RT overnight, then filtered. The filtrate was concentrated to 10.5 g of 4-benzyloxy-phenylamine (93%) as a brown solid. ¹H-NMR (400 MHz, CDCl₃): δ 7.43 (d, J=7.2 Hz, 2H), 7.38 (t, J=7.2 Hz, 1H), 7.32 (d, J=7.2 Hz, 2H), 6.83 (d, J=8.8 Hz, 2H), 6.65 (d, J=8.8 Hz, 2H), 5.00 (s, 2H), 2.94 (b, 2H): MS/ESI) m/z: 200 (M+H⁺).

To a suspension of 4-benzyloxy-phenylamine (10.0 g, 50.2 mmol) in conc. HCl (50 mL) was added a solution of sodium nitrite (3.46 g, 50.2 mmol) in water in an ice-salt bath. The mixture was stirred at 0° C. for 1 h, after which a solution of SnCl₂ (22.6 g, 100.4 mmol) in conc. HCl was added dropwise at such a rate that the reaction mixture never rose above 5°. The mixture was stiffed at RT for 2 h. The precipitate was collected by suction, washed with ethyl ether to afford 9.6 g of (4-benzyloxy-phenyl)-hydrazine hydrochloride (76%). 1H-NMR (DMSO-d6): δ 10.10 (br s, 3H), 7.43-7.33 (m, 5H), 6.99 (d, J=8.8 Hz, 2H), 6.93 (d, J=8.8 Hz, 2H), 5.03 (s, 2H); MS (ESI) m/z: 215 (M+H⁺).

A solution of (4-benzyloxy-phenyl)-hydrazine hydrochloride (7.50 g, 30 mmol) and 4,4-dimethyl-3-oxo-pentanenitrile (5.0 g, 40 mmol) in alcohol (50 mL) containing conc. HCl (5 mL) was heated to reflux overnight under N₂. After removal of the solvent, the residue was washed with ethyl ether afford 8.2 g of 3-t-butyl-1-(4-(benzyloxy)phenyl)-1H-pyrazol-5-amine (85%). 1H-NMR (DMSO-d6): δ 10.20 (br s, 3H), 7.49-7.45 (m, 4H), 7.39 (t, J=7.2 Hz, 1H), 7.34-7.29 (m, 2H), 7.19 (d, J=8.8 Hz, 2H), 5.62 (s, 1H), 5.19 (s, 2H), 1.26 (s, 9H); MS (ESI) m/z: 322 (M+H⁺).

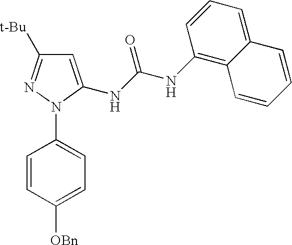

### Example 414

Using the same procedure as for Example 201, Example A15 (650 mg, 2.0 mmol) and 1-isocyanato-naphthalene (338 mg, 2.0 mmol) were combined to afford 470 mg of 1-[2-(4-Benzyloxy-phenyl)-5-t-butyl-2H-pyrazol-3-yl]-3-naphthalen-1-yl-urea (48%). 1H-NMR (DMSO-d6): δ 9.00 (s, 1H), 8.69 (s, 1H), 7.90 (d, J=7.2 Hz, 2H), 7.51-7.37 (m, 12H), 7.16 (d, J=8.8 Hz, 2H), 6.36 (s, 1H), 5.16 (s, 2H), 1.25 (s, 9H); MS (ESI) m/z: 491 (M+H⁺).

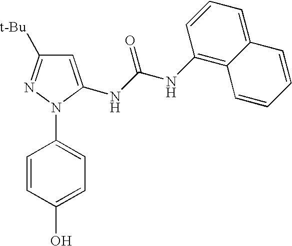

### Example 415

A mixture of Example 414 (300 mg, 0.61 mmol) and Pd/C (60 mg) in methanol (50 mL) was stirred overnight at RT under 50 psi of H₂. After the catalyst was filtered off, the filtrate was concentrated to the crude product, which was purified by column chromatography to afford 200 mg of 1-(3-t-butyl-1-(4-hydroxyphenyl)-1H-pyrazol-5-yl)-3-(naphthalen-1-yl)urea (84%); MS ESI) m/z: 401 (M+H⁺).

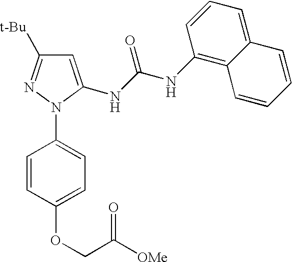

### Example 416

To a mixture of Example 415 (100 mg, 0.25 mmol) and K₂CO₂ (69 mg, 0.50 mmol), KI (50 mg, 0.30 mmol) in acetonitrile (30 mL) was added a solution of chloroacetic acid methyl ester (40 mg, 0.37 mmol) in acetonitrile (2 mL) at RT. The resultant mixture was heated to reflux for 2 h under N₂. After removal of the solvent, the residue was dissolved in CH₂Cl₂ (3×30 mL). The combined organic extracts were washed with brine, dried (Na₂SO₄), filtered, concentrated and purified via preparative HPLC to afford 55 mg of 1-(3-t-butyl-1-(4-(carbomethoxymethyl)oxyphenyl)-1H-pyrazol-5-yl)-3-(naphthalen-1-yl)urea (46%). 1H-NMR (400 MHz, DMSO-d₆): δ 9.02 (s, 1H), 8.73 (s, 1H), 7.97 (d, J=8.0 Hz, 1H), 7.89 (d, J=8.0 Hz, 2H), 7.62 (d, J=8.0 Hz, 1H), 7.53-7.51 (m, 3H), 7.42 (d, J=8.4 Hz, 2H), 7.08 (d, J=8.8 Hz, 2H), 6.36 (s, 1H), 4.85 (s, 2H), 3.69 (s, 3H), 1.25 (s, 9H); MS (ESI) m/z: 473 (M+H⁺).

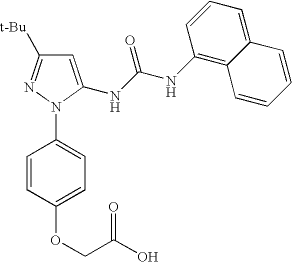

### Example 417

To a solution of Example 416 (20 mg, 0.04 mmol) in THF was added a solution of LiOH (2.0 N, 5 mL) in water at RT. The resultant mixture was stirred at RT for 3 h. After removal of the solvent, the residue was dissolved in DCM. The organic layers were washed with brine dried over Na₂SO₄ and filtered. The filtrate was concentrated to the crude product, which was purified by preparative HPLC to afford 12 mg of 1-(3-t-butyl-1-(4 (carboxy methyl)oxyphenyl)-1H-pyrazol-5-yl)-3-(naphthalen-1-yl)urea (65%). ¹H-NMR (400 MHz, DMSO-d₆): δ 13.04 (br s, 1H), 9.02 (s, 1H), 8.73 (s, 1H), 7.98 (d, J=7.2 Hz, 2H), 7.62 (d, J=8 Hz, 2H), 7.52 (t, J=7.2 Hz, 2H), 7.45-7.43 (m, 3H), 7.06 (d, J=8.8 Hz, 2H), 6.36 (s, 1H), 4.73 (s, 2H), 1.25 (s, 9H); MS (ESI) m/z: 459 (M+H⁺).

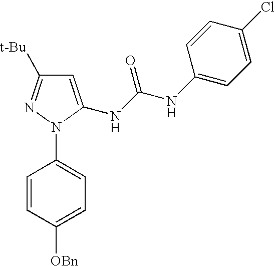

### Example 418

Using the same procedure as for Example 201, Example A15 (650 mg, 2.0 mmol) and 1-chloro-4-isocyanato-benzene (306 mg, 2.0 mmol) were combined to afford 760 mg of 1-(3-t-butyl-1-(4-(benzyloxy)phenyl)-1H-pyrazol-5-yl)-3-(4-chlorophenyl)urea (80%); MS (ESI) m/z: 474 (M+H⁺).

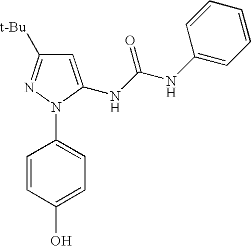

### Example 419

A mixture of Example 418 (500 mg, 1.1 mmol) and Pd/C (100 mg) in methanol (50 mL) was stirred overnight at RT. under 50 psi of H₂. After the catalyst was filtered off, the filtrate was concentrated to the crude product, which was purified by column chromatography to afford 270 mg of 1-(3-t-butyl-1-(4-hydroxyphenyl)-1H-pyrazol-5-yl)-3-phenylurea. ¹H-NMR (300 MHz, DMSO-d₆): δ 9.75 (s, 1H), 8.97 (s, 1H), 8.20 (s, 1H), 7.37 (d, J=7.8 Hz, 2H), 7.24 (d, J=7.8 Hz, 2H), 7.22 (d, J=8.1 Hz, 2H), 6.94 (t, J=7.2 Hz, 1H), 6.87 (d, J=7.8 Hz, 2H), 6.30 (s, 1H), 1.24 (s, 9H); MS (ESI) m/z: 351 (M+H⁺)

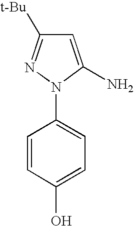

### Example 420

A mixture of Example A15 (650 mg, 2.0 mmol) and Pd/C (130 mg) in methanol (50 mL) was stirred overnight at RT under 50 psi of H₂. After the catalyst was filtered off, the filtrate was concentrated to the crude product, which was purified by column chromatography to afford 380 mg of 4-(3-t-butyl-5-amino-1H-pyrazol-1-yl)phenol (82%); MS (ESI) m/z: 232 (M+H⁺)

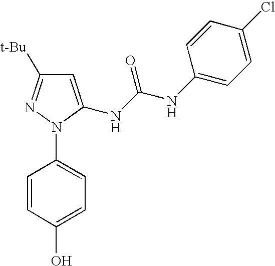

### Example 421

Using the same procedure as for Example 201, Example A15 (350 mg, 1.5 mmol) and 1-chloro-4-isocyanato-benzene (230 mg, 1.5 mmol) were combined to afford 120 mg of 1-(3-t-butyl-1-(4 hydroxyphenyl)-1H-pyrazol-5-yl)-3-(4-chlorophenyl)urea (20%). ¹H-NMR (300 MHz, DMSO-d₆): δ 9.82 (br s, 1H), 9.12 (s, 1H), 8.25 (s, 1H), 7.41 (d, J=9.0 Hz, 2H), 7.28 (d, J=9.0 Hz, 2H), 7.24 (d, J=8.7 Hz, 2H), 6.86 (d, J=8.7 Hz, 2H), 6.30 (s, 1H), 1.24 (s, 9H); MS (ESI) m/z: 385 (M+H⁺)

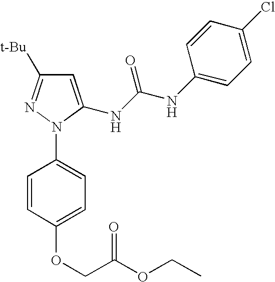

### Example 422

Using the same procedure as for Example 416, Example 421 (120 mg, 0.31 mmol) and chloroacetic acid ethyl ester (76.5 mg, 0.62 mmol) were combined to afford 110 mg of 1-(3-t-butyl-1-(4-(carbomethoxymethyl)oxyphenyl)-1H-pyrazol-5-yl)-3-(4-chlorophenyl-1-yl)urea (75%) as a white solid. ¹H-NMR (300 MHz, DMSO-d₆): δ 9.09 (s, 1H), 8.31 (s, 1H), 7.40 (d, J=5.4 Hz, 2H), 7.34 (d, J=5.4 Hz, 2H), 7.27 (d, J=9.0 Hz, 2H), 7.04 (d, J=9.0 Hz, 2H), 6.30 (s, 1H), 4.81 (s, 2H), 4.16 (q, J=7.2 Hz, 2H), 1.24 (s, 9H), 1.20 (t, J=7.2 Hz, 3H); MS (ESI) m/z: 471 (M+H⁺)

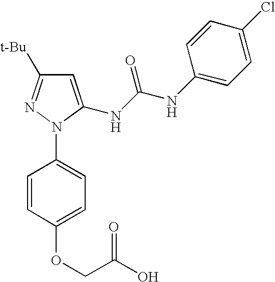

### Example 423

Using the same procedure as for Example 417, Example 422 (60 mg, 0.13 mmol) was saponified to afford 40 mg of 1-(3-t-butyl-1-(4-(carboxymethyl)oxyphenyl)-1H-pyrazol-5-yl)-3-(4-chlorophenyl-1-yl)urea (71%) as a white solid. ¹H-NMR (300 MHz, DMSO-d₆): δ 9.14 (s, 1H), 8.35 (s, 1H), 7.40 (d, J=6.9 Hz, 2H), 7.37 (d, J=6.9 Hz, 2H), 7.27 (d, J=9.0 Hz, 2H), 7.02 (d, J=9.0 Hz, 2H), 6.30 (s, 1H), 4.71 (s, 2H), 1.23 (s, 9H); MS (ESI) m/z: 443 (M+H⁺)

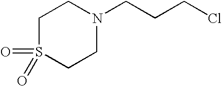

### Example A16

To a mixture of thiomorpholine (500 mg, 3.7 mmol), K₂CO₃ (1.0 g, 7.5 mmol) in acetonitril (50 mL) was added 1-bromo-3-chloro-propane (780 mg, 5.0 mmol) at RT. The mixture was stirred at RT for 3 h. After removal of the solvent, the residue was dissolved in dichloromethane. The organic layer was washed with brine, dried over Na₂SO₄ and filtered. The filtrate was concentrated to the crude product, which was added a solution of HCl/MeOH. After removal of the solvent, the residue was washed with Et₂O to afford 510 mg of 4-(3-chloro-propyl)-thiomorpholine-1,1-dioxide (66%). ¹H NMR (400 MHz, D₂O) δ: 3.61 (br s, 4H), 3.31 (br s, 6H), 2.92 (br s, 3H), 2.15 (br s, 2H).

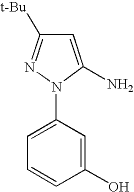

### Example A17

Using the same procedure as for Example TT, m-methoxyphenylhydrazine (40 mmol) and 4,4-dimethyl-3-oxo-pentanenitrile (5.0 g, 40 mmol) were combined to afford 3-(3-t-butyl-5-amino-1H-pyrazol-1-yl)phenol, which was used without further purification.

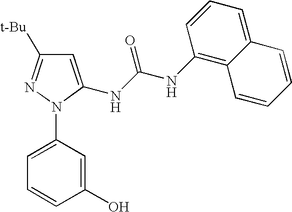

### Example 424

To a solution of Example 201, Example A17 (2 mmol) and 1-isocyanato-naphthalene (338 mg, 2.0 mmol) were combined to yield 1-(3-t-butyl-1-(3-hydroxyphenyl)-1H-pyrazol-5-yl)-3-(naphthalen-1-yl)urea, which was used without further purification.

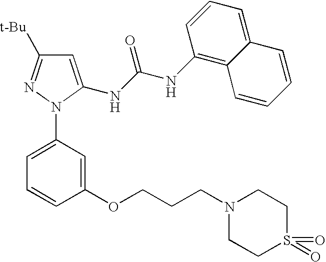

### Example 425

To a solution of Example 424 (100 mg, 0.25 mmol) and K₂CO₃ (68 mg, 0.5 mmol) in acetonitril (10 mL) was added Example A16 (630 mg, 0.30 mmol). The resulting mixture was stirred at 50° C. for 3 h. After removal of the solvent, the residue was dissolved in CH₂Cl₂. The combined organic extracts were washed with brine, dried (Na₂SO₄), filtered, concentrated and purified via preparative HPLC to afford 55 mg of 1-(5-t-butyl-2-{3-[3-(1,1-dioxo-1λ6-thiomorpholin-4-yl)-propoxy]-phenyl}-2H-pyrazol-3-yl)-3-naphthalen-1-yl-urea (38%). ¹H-NMR (400 MHz, CDCl₃) δ: 7.87 (d, J=6.8 Hz, 1H), 7.83 (d, J=7.6 Hz, 1H), 7.74 (d, J=8.4 Hz, 1H), 7.60-7.47 (m, 3H), 7.42 (t, J=7.6 Hz, 1H), 7.11 (m, 1H), 6.95 (s, 2H), 6.79-6.74 (m, 2H), 6.48 (s, 1H), 3.96 (br s, 2H), 3.51 (br s, 4H), 3.01 (br s, 4H), 2.67 (br s, 2H), 1.92 (br s, 2H), 1.35 (s, 9H). MS (ESI) m/z: 576 (M+H⁺).

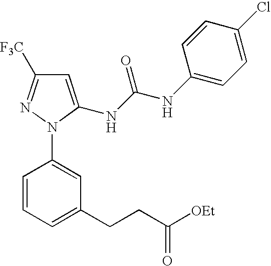

### Example 426

Using the same procedure as for Example 311, 4,4,4-trifluoro-3-oxo-butyronitrile (from Example WW, 1.37 g, 10.0 mmol) was transformed to 4,4,4-trifluoro-3-oxo-butyrimidic acid ethyl ester hydrochloride (1.1 g, 5.0 mmol), which was combined with 1-chloro-4-isocyanato-benzene to afford 970 mg 1-(4-chloro-phenyl)-3-(1-ethoxy-4,4,4-trifluoro-3-oxo-but-1-enyl)-urea (MS (ESI) m/z: 337 (M+H⁺)). This was combined with 3-(3-hydrazino-phenyl)-propionic acid ethyl ester (from Example EEE, 500 mg, 2.05 mmol) to yield 650 mg of 3-{3-(5-[3-(4-chloro-phenyl)-ureido]-3-trifluoromethyl-pyrazol-1-yl}-phenyl)-propionic acid ethyl ester. ¹H NMR (400 MHz, DMSO-d₆): 9.19 (s, 1H), 8.70 (s, 1H), 7.52-7.41 (m, 6H), 7.29 (d, J=8.8 Hz, 2H), 6.84 (s, 1H), 4.01 (q, J=7.2 Hz, 2H), 2.92 (t, J=7.6 Hz, 2H), 2.64 (t, J=7.6 Hz, 2H), 1.11 (t, J=7.2 Hz, 3H). MS (ESI) m/z: 481 (M+H⁺).

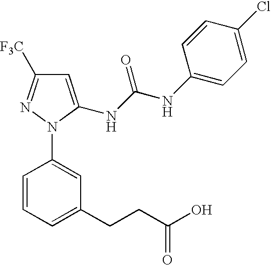

### Example 427

Using the same procedure as for Example 203, Example 426 (150 mg, 0.31 mmol) was saponified to afford 110 mg of 3-(3-(5-(3-(4 chlorophenyl)ureido)-3-(trifluoromethyl)-1H-pyrazol-1-yl)phenyl)propanoic acid. ¹H NMR (400 MHz, DMSO-d₆): 12.15 (br s, 1H), 9.36 (s, 1H), 8.79 (s, 1H), 7.50-7.38 (m, 6H), 7.29 (d, J=8.8 Hz, 2H), 6.84 (s, 1H), 2.90 (t, J=7.2 Hz, 2H), 2.57 (t, J=7.6 Hz, 2H). MS (ESI) m/z: 453 (M+H⁺).

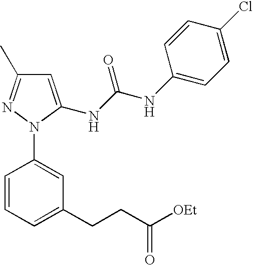

### Example 428

Using the same procedure as for Example 311, 3-oxo-butyronitrile (from Example UU, 830 mg, 10.0 mmol) was transformed to 3-oxo-butyrimidic acid ethyl ester hydrochloride (900 mg, 5.4 mmol), which was combined with 1-chloro-4-isocyanato-benzene (1.1 g, 7.2 mmol) to afford 1.3 g of 1-(4-chloro-phenyl)-3-(1-ethoxy-3-oxo-but-1-enyl)-urea (MS (ESI) m/z: 337 (M+H⁺)). This was combined with 3-(3-hydrazino-phenyl)-propionic acid ethyl ester (from Example EEE, 500 mg, 2.05 mmol) to yield 750 mg of 3-(3-{5-[3-(4-chloro-phenyl)-ureido)-3-methyl-pyrazol-1-yl}-phenyl)-propionic acid ethyl ester. ¹H NMR (400 MHz, CDCl₃-d₆): 7.39-7.32 (m, 3H), 7.42 (d, J=8.4 Hz, 2H), 7.19 (d, J=8.4 Hz, 2H), 7.13 (d, J=8.0 Hz, 1H), 6.78 (d, J=7.6 Hz, 1H), 6.62 (s, 1H), 6.40 (s, 1H), 4.11 (q, J=7.2 Hz, 2H), 2.86 (t, J=7.6 Hz, 2H), 2.56 (t, J=7.6 Hz, 2H), 1.20 (t, J=7.2 Hz, 3H). MS (ESI) m/z: 427 (M+H⁺).

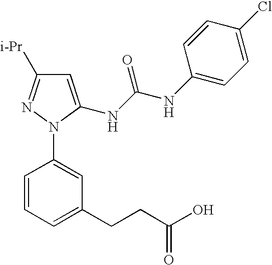

### Example 429

Using the same procedure as for Example 203, Example 313 (200 mg, 0.43 mmol) was saponified to afford 140 mg of 3-(3-(5-(3-(4-chlorophenyl)ureido)-3-isopropyl-1H-pyrazol-1-yl)phenyl)propanoic acid ¹H NMR (400 MHz, CD₄O-d₄): 7.51 (t, J=8.0 Hz, 1H), 7.39-7.35 (m, 5H), 7.24 (d, J=8.8 Hz, 2H), 6.50 (s, 1H), 3.04-2.98 (m, 3H), 2.67 (t, J=7.6 Hz, 2H), 1.31 (d, J=6.8 Hz, 3H). MS (ESI) m/z: 427 (M+H⁺).

The following compounds were synthesized.

| CIP Example | |
|---|---|
| Number | Compound Name |
| 430 | 2-(3-(5-(3-(2,3-dichlorophenyl)ureido)-3-phenyl-1H-pyrazol-1- |
| | yl)phenyl)acetic acid |
| 431 | 2-(3-(5-(3-(2,3-dichlorophenyl)ureido)-3-(4-fluorophenyl)-1H-pyrazol-1- |
| | yl)phenyl)acetic acid |
| 432 | 2-(3-(5-(3-(2,3-dichlorophenyl)ureido)-3-(3-fluorophenyl)-1H- |
| | pyrazol-1-yl)phenyl)acetic acid |
| 433 | 2-(3-(5-(3-(2,3-dichlorophenyl)ureido)-3-(2-fluorophenyl)-1H- |
| | pyrazol-1-yl)phenyl)acetic acid |
| 434 | 2-(3-(3-cyclopentyl-5-(3-(2,3-dichlorophenyl)ureido)-1H-pyrazol-1- |
| | yl)phenyl)acetic acid |
| 435 | 2-(3-(5-(3-(2,3-dichlorophenyl)ureido)-3-(thiophen-2-yl)-1H- |
| | pyrazol-1-yl)phenyl)acetic acid |
| 436 | 2-(3-(5-(3-(2,3-dichlorophenyl)ureido)-3-(thiophen-3-yl)-1H- |
| | pyrazol-1-yl)phenyl)acetic acid |
| 437 | ethyl 2-(4-(3-cyclopentyl-5-(3-(2,3-dichlorophenyl)ureido)-1H- |
| | pyrazol-1-yl)phenyl)acetate |
| 438 | 2-(4-(5-(3-(2,3-dichlorophenyl)ureido)-3-phenyl-1H-pyrazol-1- |
| | yl)phenyl)acetic acid |
| 439 | 2-(4-(5-(3-(2,3-dichlorophenyl)ureido)-3-(3-fluorophenyl)-1H- |
| | pyrazol-1-yl)phenyl)acetic acid |
| 440 | 2-(4-(5-(3-(2,3-dichlorophenyl)ureido)-3-(2-fluorophenyl)-1H- |
| | pyrazol-1-yl)phenyl)acetic acid |
| 441 | 2-(4-(3-cyclopentyl-5-(3-(2,3-dichlorophenyl)ureido)-1H-pyrazol-1- |
| | yl)phenyl)acetic acid |
| 442 | 2-(4-(5-(3-(2,3-dichlorophenyl)ureido)-3-(thiophen-2-yl)-1H- |
| | pyrazol-1-yl)phenyl)acetic acid |
| 443 | 2-(4-(5-(3-(2,3-dichlorophenyl)ureido)-3-(thiophen-2-yl)-1H- |
| | pyrazol-1-yl)phenyl)propanoic acid |
| 444 | 2-(4-(5-(3-(2,3-dichlorophenyl)ureido)-3-(thiophen-3-yl)-1H- |
| | pyrazol-1-yl)phenyl)acetic acid |
| 445 | methyl 2-(4-(5-(3-(2,3-dichlorophenyl)ureido)-3-(thiophen-3-yl)- |
| | 1H-pyrazol-1-yl)phenyl)acetate |
| 446 | 1-(1-(3-(2-amino-2-oxoethyl)phenyl)-3-cyclopentyl-1H-pyrazol-5- |
| | yl)-3-(2,3-dichlorophenyl)urea |
| 447 | 1-(1-(3-(2-amino-2-oxoethyl)phenyl)-3-phenyl-1H-pyrazol-5-yl)-3- |
| | (2,3-dichlorophenyl)urea |
| 448 | 1-(1-(3-(2-amino-2-oxoethyl)phenyl)-3-(4-fluorophenyl)-1H- |
| | pyrazol-5-yl)-3-(2,3-dichlorophenyl)urea |
| 449 | 1-(1-(3-(2-amino-2-oxoethyl)phenyl)-3-(3-fluorophenyl)-1H- |
| | pyrazol-5-yl)-3-(2,3-dichlorophenyl)urea |
| 450 | 1-(1-(3-(2-amino-2-oxoethyl)phenyl)-3-(2-fluorophenyl)-1H- |
| | pyrazol-5-yl)-3-(2,3-dichlorophenyl)urea |
| 451 | 1-(1-(3-(2-amino-2-oxoethyl)phenyl)-3-(thiophen-2-yl)-1H-pyrazol- |
| | 5-yl)-3-(2,3-dichlorophenyl)urea |
| 452 | 1-(1-(3-(2-amino-2-oxoethyl)phenyl)-3-(thiophen-3-yl)-1H-pyrazol- |
| | 5-yl)-3-(2,3-dichlorophenyl)urea |
| 453 | 1-(1-(3-(1-amino-1-oxopropan-2-yl)phenyl)-3-(thiophen-3-yl)-1H- |
| | pyrazol-5-yl)-3-(2,3-dichlorophenyl)urea |
| 454 | 1-(2,3-dichlorophenyl)-3-(1-(3-(2-(2-hydroxyethylamino)-2- |
| | oxoethyl)phenyl)-3-phenyl-1H-pyrazol-5-yl)urea |
| 455 | 1-(2,3-dichlorophenyl)-3-(3-(3-fluorophenyl)-1-(3-(2-(2- |
| | hydroxyethylamino)-2-oxoethyl)phenyl)-1H-pyrazol-5-yl)urea |
| 456 | 1-(2,3-dichlorophenyl)-3-(3-(2-fluorophenyl)-1-(3-(2-(2- |
| | hydroxyethylamino)-2-oxoethyl)phenyl)-1H-pyrazol-5-yl)urea |
| 457 | 1-(2,3-dichlorophenyl)-3-(1-(3-(2-(2-hydroxyethylamino)-2- |
| | oxoethyl)phenyl)-3-(thiophen-2-yl)-1H-pyrazol-5-yl)urea |
| 458 | 1-(2,3-dichlorophenyl)-3-(1-(3-(2-(2-hydroxyethylamino)-2- |
| | oxoethyl)phenyl)-3-(thiophen-3-yl)-1H-pyrazol-5-yl)urea |
| 459 | 1-(2,3-dichlorophenyl)-3-(1-(3-(2-(2,3-dihydroxypropylamino)-2- |
| | oxoethyl)phenyl)-3-(2-fluorophenyl)-1H-pyrazol-5-yl)urea |
| 460 | 1-(2,3-dichlorophenyl)-3-(1-(3-(2-(2,3-dihydroxypropylamino)-2- |
| | oxoethyl)phenyl)-3-(thiophen-2-yl)-1H-pyrazol-5-yl)urea |
| 461 | 1-(2,3-dichlorophenyl)-3-(1-(3-(2-(1,3-dihydroxypropan-2- |
| | ylamino)-2-oxoethyl)phenyl)-3-(2-fluorophenyl)-1H-pyrazol-5- |
| | yl)urea |
| 462 | 1-(2,3-dichlorophenyl)-3-(1-(3-(2-(1,3-dihydroxypropan-2- |
| | ylamino)-2-oxoethyl)phenyl)-3-(thiophen-2-yl)-1H-pyrazol-5- |
| | yl)urea |
| 463 | 1-(2,3-dichlorophenyl)-3-(1-(3-(2-((S)-3-hydroxypyrrolidin-1-yl)-2- |
| | oxoethyl)phenyl)-3-(thiophen-2-yl)-1H-pyrazol-5-yl)urea |
| 464 | 1-(3-tert-butyl-1-(3-(2-((R)-3-(dimethylamino)pyrrolidin-1-yl)-2- |
| | oxoethyl)phenyl)-1H-pyrazol-5-yl)-3-(2,3-dichlorophenyl)urea |
| 465 | 1-(3-tert-butyl-1-(3-(2-((S)-3-(dimethylamino)pyrrolidin-1-yl)-2- |
| | oxoethyl)phenyl)-1H-pyrazol-5-yl)-3-(2,3-dichlorophenyl)urea |
| 466 | 1-(1-(4-(2-amino-2-oxoethyl)phenyl)-3-(2-fluorophenyl)-1H- |
| | pyrazol-5-yl)-3-(2,3-dichlorophenyl)urea |
| 467 | 1-(1-(4-(2-amino-2-oxoethyl)phenyl)-3-cyclopentyl-1H-pyrazol-5- |
| | yl)-3-(2,3-dichlorophenyl)urea |
| 468 | 1-(1-(4-(2-amino-2-oxoethyl)phenyl)-3-(thiophen-2-yl)-1H-pyrazol- |
| | 5-yl)-3-(2,3-dichlorophenyl)urea |
| 469 | 1-(1-(4-(2-amino-2-oxoethyl)phenyl)-3-(thiophen-3-yl)-1H-pyrazol- |
| | 5-yl)-3-(2,3-dichlorophenyl)urea |
| 470 | 1-(2,3-dichlorophenyl)-3-(1-(4-(2-(2-hydroxyethylamino)-2- |
| | oxoethyl)phenyl)-3-phenyl-1H-pyrazol-5-yl)urea |
| 471 | 1-(2,3-dichlorophenyl)-3-(1-(4-(2-(2,3-dihydroxypropylamino)-2- |
| | oxoethyl)phenyl)-3-(2-fluorophenyl)-1H-pyrazol-5-yl)urea |
| 472 | 1-(2,3-dichlorophenyl)-3-(1-(4-(2-(2,3-dihydroxypropylamino)-2- |
| | oxoethyl)phenyl)-3-(thiophen-2-yl)-1H-pyrazol-5-yl)urea |
| 473 | 1-(2,3-dichlorophenyl)-3-(1-(4-(2-(1,3-dihydroxypropan-2- |
| | ylamino)-2-oxoethyl)phenyl)-3-(2-fluorophenyl)-1H-pyrazol-5- |
| | yl)urea |
| 474 | 1-(2,3-dichlorophenyl)-3-(1-(4-(2-(1,3-dihydroxypropan-2- |
| | ylamino)-2-oxoethyl)phenyl)-3-(thiophen-2-yl)-1H-pyrazol-5- |
| | yl)urea |
| 475 | 1-(2,3-dichlorophenyl)-3-(1-(4-(2-(4-methylpiperazin-1-yl)-2- |
| | oxoethyl)phenyl)-3-phenyl-1H-pyrazol-5-yl)urea |
| 476 | 1-(2-(4-(3-tert-butyl-5-(3-(2,3-dichlorophenyl)ureido)-1H-pyrazol-1- |
| | yl)phenyl)acetyl)piperidine-3-carboxylic acid |
| 477 | (R)-1-(2,3-dichlorophenyl)-3-(1-(4-(2-(2- |
| | (hydroxymethyl)pyrrolidin-1-yl)-2-oxoethyl)phenyl)-3-phenyl-1H- |
| | pyrazol-5-yl)urea |
| 478 | (S)-1-(3-tert-butyl-1-(4-(2-(3-hydroxypyrrolidin-1-yl)-2- |
| | oxoethyl)phenyl)-1H-pyrazol-5-yl)-3-(2,3-dichlorophenyl)urea |
| 479 | 1-(2,3-dichlorophenyl)-3-(3-(2-fluorophenyl)-1-(4-(2-((S)-3- |
| | hydroxypyrrolidin-1-yl)-2-oxoethyl)phenyl)-1H-pyrazol-5-yl)urea |
| 480 | (R)-1-(3-tert-butyl-1-(4-(2-(3-(dimethylamino)pyrrolidin-1-yl)-2- |
| | oxoethyl)phenyl)-1H-pyrazol-5-yl)-3-(2,3-dichlorophenyl)urea |
| 481 | (R)-1-(3-tert-butyl-1-(4-(2-(3-(dimethylamino)pyrrolidin-1-yl)-2- |
| | oxoethyl)phenyl)-1H-pyrazol-5-yl)-3-(2,3-dichlorophenyl)urea |
| 482 | 1-(3-tert-butyl-1-(3-((2,4,5-trioxoimidazolidin-1-yl)methyl)phenyl)- |
| | 1H-pyrazol-5-yl)-3-(2,3-dichlorophenyl)urea |
| 483 | [1,1′-biphenyl]-2′-(3-(4-(1-oxoisoindolin-4-yl)phenyl)ureido)-4′- |
| | (trifluoromethyl)-3-acetic acid amide |
| 484 | 1-(2,3-dichlorophenyl)-3-(1-(3-(hydroxymethyl)phenyl)-3-phenyl- |
| | 1H-pyraol-5-yl)urea |
| 485 | 1-(2,3-dichlorophenyl)-3-(3-(2-fluorophenyl)-1-(3- |
| | (hydroxymethyl)phenyl)-1H-pyrazol-5-yl)urea |
| 486 | 1-(2,3-dichlorophenyl)-3-(1-(3-(hydroxymethyl)phenyl)-3- |
| | (thiophen-2-yl)-1H-pyrazol-5-yl)urea |
| 487 | 1-(3-cyclopentyl-1-(3-(2-(2,3-dihydroxypropylamino)-2- |
| | oxoethyl)phenyl)-1H-pyrazol-5-yl)-3-(2,3-dichlorophenyl)urea |
| 488 | 1-(3-cyclopentyl-1-(3-(2-(2-hydroxyethylamino)-2- |
| | oxoethyl)phenyl)-1H-pyrazol-5-yl)-3-(2,3-dichlorophenyl)urea |
| 489 | 1-(1-(3-(3-amino-2-methyl-3-oxopropyl)phenyl)-3-(2-fluorophenyl)- |
| | 1H-pyrazol-5-yl)-3-(2,3-dichlorophenyl)urea |
| 490 | 3-(3-(5-(3-(2,3-dichlorophenyl)ureido)-3-(thiophen-2-yl)-1H- |
| | pyrazol-1-yl)phenyl)-2-methylpropanoic acid |
| 491 | 1-(1-(3-(2-amino-2-oxoethyl)phenyl)-3-tert-butyl-1H-pyrazol-5-yl)- |
| | 3-(2,3,4-trifluorophenyl)urea |
| 492 | 2-(4-(3-tert-butyl-5-(3-(2,3,4-trifluorophenyl)ureido)-1H-pyrazol-1- |
| | yl)phenyl)acetic acid |
| 493 | 1-(1-(3-(hydroxymethyl)phenyl)-3-(thiophen-2-yl)-1H-pyrazol-5- |
| | yl)-3-(2,3,4-trifluorophenyl)urea |
| 494 | 1-(1-(3-(2-amino-2-oxoethyl)phenyl)-3-tert-butyl-1H-pyrazol-5-yl)- |
| | 3-(2,4,5-trifluorophenyl)urea |
| 495 | 1-(1-(3-(2-amino-2-oxoethyl)phenyl)-3-tert-butyl-1H-pyrazol-5-yl)- |
| | 3-(2,3-difluorophenyl)urea |
| 496 | 1-(1-(3-(2-amino-2-oxoethyl)phenyl)-3-tert-butyl-1H-pyrazol-5-yl)- |
| | 3-(2,4-difluorophenyl)urea |
| 497 | 2-(4-(3-tert-butyl-5-(3-(2,4-difluorophenyl)ureido)-1H-pyrazol-1- |
| | yl)phenyl)acetic acid |
| 498 | 1-(1-(4-(2-amino-2-oxoethyl)phenyl)-3-tert-butyl-1H-pyrazol-5-yl)- |
| | 3-(2,4-difluorophenyl)urea |
| 499 | 1-(3-tert-butyl-1-(3-cyanophenyl)-1H-pyrazol-5-yl)-3-(2,4- |
| | difluorophenyl)urea |
| 500 | 1-(1-(3-(2-amino-2-oxoethyl)phenyl)-3-tert-butyl-1H-pyrazol-5-yl)- |
| | 3-(3-(pyridin-3-yloxy)phenyl)urea |
| 501 | 1-(1-(3-(2-amino-2-oxoethyl)phenyl)-3-phenyl-1H-pyrazol-5-yl)-3- |
| | (3-(pyridin-3-yloxy)phenyl)urea |
| 502 | 1-(1-(3-(2-amino-2-oxoethyl)phenyl)-3-(thiophen-2-yl)-1H-pyrazol- |
| | 5-yl)-3-(3-(pyridin-3-yloxy)phenyl)urea |
| 503 | 1-(1-(3-(2-amino-2-oxoethyl)phenyl)-3-(trifluoromethyl)-1H- |
| | pyrazol-5-yl)-3-(3-(pyridin-3-yloxy)phenyl)urea |
| 504 | 1-(1-(3-(2-amino-2-oxoethyl)phenyl)-3-tert-butyl-1H-pyrazol-5-yl)- |
| | 3-(3-(pyrazin-2-yloxy)phenyl)urea |
| 505 | 1-(1-(3-(2-amino-2-oxoethyl)phenyl)-3-tert-butyl-1H-pyrazol-5-yl)- |
| | 3-(4-(pyridin-4-yloxy)phenyl)urea |
| 506 | 1-(1-(3-(2-amino-2-oxoethyl)phenyl)-3-tert-butyl-1H-pyrazol-5-yl)- |
| | 3-(4-(2-(methylcarbamoyl)pyridin-4-yloxy)phenyl)urea |
| 507 | 1-(1-(3-(2-amino-2-oxoethyl)phenyl)-3-tert-butyl-1H-pyrazol-5-yl)- |
| | 3-(3-(pyridin-3-yl)phenyl)urea |
| 508 | 1-(1-(3-(2-amino-2-oxoethyl)phenyl)-3-tert-butyl-1H-pyrazol-5-yl)- |
| | 3-(3-(6-aminopyridin-3-yl)phenyl)urea |
| 509 | 1-(1-(3-(2-amino-2-oxoethyl)phenyl)-3-tert-butyl-1H-pyrazol-5-yl)- |
| | 3-(3-(pyrazin-2-yl)phenyl)urea |
| 510 | 1-(1-(3-(2-amino-2-oxoethyl)phenyl)-3-tert-butyl-1H-pyrazol-5-yl)- |
| | 3-(4-(1-oxoisoindolin-4-yl)phenyl)urea |
| 511 | 1-{5-t-butyl-2-[3-(4-methylene-1,1,3-trioxo-[1,2,5]thiadiazolidin-2- |
| | ylmethyl)-phenyl]-2H-pyrazol-3-yl}-3-(2,3-dichlorophenyl)-urea |
| 512 | 1-(1-(3-(2-amino-2-oxoethyl)phenyl)-3-cyclopentyl-1H-pyrazol-5- |
| | yl)-3-(4-(1-oxoisoindolin-4-yl)phenyl)urea |
| 513 | 1-(1-(3-(2-amino-2-oxoethyl)phenyl)-3-tert-butyl-1H-pyrazol-5-yl)- |
| | 3-(3-(8-methyl-7-oxo-7,8-dihydropyrido[2,3-d]pyrimidin-6- |
| | yl)phenyl)urea |
| 514 | 1-(1-(3-(2-amino-2-oxoethyl)phenyl)-3-cyclopentyl-1H-pyrazol-5- |
| | yl)-3-(3-(8-methyl-7-oxo-7,8-dihydropyrido[2,3-d]pyrimidin-6- |
| | yl)phenyl)urea |
| 515 | 1-(1-(3-(2-amino-2-oxoethyl)phenyl)-3-tert-butyl-1H-pyrazol-5-yl)- |
| | 3-(4-methyl-3-(pyrimidin-2-ylamino)phenyl)urea |
| 516 | 1-(1-(3-(2-amino-2-oxoethyl)phenyl)-3-tert-butyl-1H-pyrazol-5-yl)- |
| | 3-(4-methyl-3-(4-(pyridin-3-yl)pyrimidin-2-ylamino)phenyl)urea |
| 517 | 1-(3-tert-butyl-1-(3-(2-(2,3-dihydroxypropylamino)-2- |
| | oxoethyl)phenyl)-1H-pyrazol-5-yl)-3-(4-methyl-3-(4-(pyridin-3- |
| | yl)pyrimidin-2-ylamino)phenyl)urea |

### Affinity and Biological Assessment of P38-Alpha Kinase Inhibitors

A fluorescence binding assay is used to detect binding of inhibitors of Formula I with unphosphorylated p38-alpha kinase as previously described: see J. Regan et al, Journal of Medicinal Chemistry (2002) 45:2994.

### 1. P38 MAP Kinase Binding Assay

The binding affinities of small molecule modulators for p38 MAP kinase were determined using a competition assay with SKF 86002 as a fluorescent probe, modified based on published methods (C. Pargellis, et al Nature Structural Biology (2002) 9, 268-272. J. Regan, et al J. Med. Chem. (2002) 45, 2994-3008). Briefly, SKF 86002, a potent inhibitor of p38 kinase (K_{d}=180 nM) displays an emission fluorescence around 420 nm when excitated at 340 nm upon its binding to the kinase. Thus, the binding affinity of an inhibitor for p38 kinase can be measured by its ability to decrease the fluorescence from SKF 86002. The assay was performed in a 384 plate (Greiner uclear 384 plate) on a Polarstar Optima plate reader (BMG). Typically, the reaction mixture contained 1 μM SKF 86002, 80 nM p38 kinase and various concentrations of an inhibitor in 20 mM Bis-Tris Propane buffer, pH 7, containing 0.15% (w/v) n-octylglucoside and 2 mM EDTA in a final volume of 65 μl. The reaction was initiated by addition of the enzyme. The plate was incubated at room temperature (˜25° C.) for 2 hours before reading at emission of 420 nm and excitation at 340 nm. By comparison of rfu (relative fluorescence unit) values with that of a control (in the absence of an inhibitor), the percentage of inhibition at each concentration of the inhibitor was calculated. IC₅₀ value for the inhibitor was calculated from the % inhibition values obtained at a range of concentrations of the inhibitor using Prism. When time-dependent inhibition was assessed, the plate was read at multiple reaction times such as 0.5, 1, 2, 3, 4 and 6 hours. The IC₅₀ values were calculated at the each time point. An inhibition was assigned as time-dependent if the IC₅₀ values decrease with the reaction time (more than two-fold in four hours). This is illustrated below in Table 1.

TABLE 1

| | | Time- |
|---|---|---|
| Example # | IC50, nM | dependent |
| 1 | 292 | Yes |
| 2 | 997 | No |
| 2 | 317 | No |
| 3 | 231 | Yes |
| 4 | 57 | Yes |
| 5 | 1107 | No |
| 6 | 238 | Yes |
| 7 | 80 | Yes |
| 8 | 66 | Yes |
| 9 | 859 | No |
| 10 | 2800 | No |
| 11 | 2153 | No |
| 12 | ~10000 | No |
| 13 | 384 | Yes |
| 15 | 949 | No |
| 19 | ~10000 | No |
| 21 | 48 | Yes |
| 22 | 666 | No |
| 25 | 151 | Yes |
| 26 | 68 | Yes |
| 29 | 45 | Yes |
| 30 | 87 | Yes |
| 31 | 50 | Yes |
| 32 | 113 | Yes |
| 37 | 497 | No |
| 38 | 508 | No |
| 41 | 75 | Yes |
| 42 | 373 | No |
| 43 | 642 | No |
| 45 | 1855 | No |
| 46 | 1741 | No |
| 47 | 2458 | No |
| 48 | 3300 | No |
| 57 | 239 | Yes |

IC50 values obtained at 2 hours reaction time

### P-38 Alpha Kinase Assay (Spectrophometric Assay)

Activity of phosphorylated p-38 kinase was determined by following the production of ADP from the kinase reaction through coupling with the pyruvate kinase/lactate dehydrogenase system (e.g., Schindler, et al. Science (2000) 289, 1938-1942). In this assay, the oxidation of NADH was continuously measured spectrophometrically. The reaction mixture (100 μl) contained phospho p-38 alpha kinase (3.3 mM. Panvera), peptide substrate (IPTSPITTTYFFFKKK-OH, 0.2 mM), ATP (0.3 mM), MgCl₂ (10 mM), pyruvate kinase (8 units. Sigma), lactate dehydrogenase (13 units. Sigma), phosphoenol pyruvate (1 mM), and NADH (0.28 mM) in 65 mM Tris buffer, pH 7.5, containing 3.5% DMSO and 150 uM n-Dodecyl-B-D-maltopyranoside. The reaction was initiated by adding ATP. The absorption at 340 nm was monitored continuously for up to 4 hours at 30° C. on Polarstar Optima plate reader (BMG). The kinase activity (reaction rate) was calculated from the slope at the time frame from 1.5 h to 2 h. Under these conditions, a turn over number (k_{cat}) of ˜1 s⁻¹ was obtained. The reaction rates calculated from different time frames such as 0.5 min to 0.5 h, 0.5 h to 1 h, 1.5 h to 2 h or 2.5 h to 3 h were generally constant.

For inhibition determinations, test compounds were incubated with the reaction mixture for ˜5 min before adding ATP to start the reaction. Percentage of inhibition was obtained by comparison of reaction rate with that of a control well containing no test compound. IC50 values were calculated from a series of % inhibition values determined at a range of concentrations of each inhibitor using Prism to process the data and fit inhibition curves. Generally, the rates obtained at the time frame of 1.5 h to 2 h were used for these calculations. In assessing whether inhibition of a test compound was time-dependent (i.e., greater inhibition with a longer incubation time), the values of % inhibition and/or IC₅₀ values obtained from other time frames were also calculated for the inhibitor.

TABLE 2

| Example # | IC50, uM | % inhibition @ concentration, uM |
|---|---|---|
| 1 | 0.067 | |
| 2 | 0.29 | |
| 3 | 0.019 | |
| 4 | 0.609 | |
| 5 | 0.514 | |
| 6 | 0.155 | |
| 7 | 0.165 | |
| 9 | 0.355 | |
| 10 | | 83% @ 10 |
| 11 | 0.953 | |
| 12 | | 70% @ 10 |
| 13 | 0.269 | |
| 14 | 0.096 | |
| 15 | 0.53 | |
| 17 | | 40% @ 10 |
| 18 | | 60% @ 10 |
| 21 | 0.171 | |
| 22 | 0.445 | |
| 25 | 0.055 | |
| 26 | 0.19 | |
| 29 | 0.011 | |
| 30 | 0.251 | |
| 31 | 0.056 | |
| 32 | 0.307 | |
| 38 | 0.51 | |
| 39 | 0.012 | |
| 40 | 0.055 | |
| 41 | 0.013 | |
| 42 | 0.425 | |
| 43 | 7.5 | |
| 45 | 0.48 | |
| 46 | 1 | |
| 47 | 0.295 | |
| 48 | 2 | |
| 49 | 0.071 | |
| 51 | 0.033 | |
| 52 | 0.416 | |
| 53 | 0.109 | |
| 54 | | 68% @ 1.0 |
| 55 | 0.74 | |
| 57 | 0.782 | |
| 58 | 0.172 | |
| 59 | 0.709 | |
| 60 | 0.264 | |
| D | 0.179 | |
| F | 0.437 | |
| Q | 0.284 | |

TABLE 3

| | | % Inhibition @ concentration, |
|---|---|---|
| Example # | IC50, uM | uM |
| 145 | 1.3 | |
| 146 | | 9% @ 10 |
| 147 | | 27% @ 10 |
| 150 | | 53% @ 10 |
| 154 | | 21% @ 10 |
| 155 | | 58% @ 10 |
| 160 | 0.044 | |
| 161 | 0.1 | |
| 162 | 0.65 | |
| 163 | 0.464 | |
| 196 | 0.028 | |
| 197 | 0.243 | |
| 198 | 0.137 | |
| 199 | 0.684 | |
| 200 | | 73% @ 1.0 |
| 201 | 0.029 | |
| 202 | 1.9 | |
| 203 | 0.328 | |
| 204 | 0.008 | |
| 206 | 0.013 | |
| 207 | 0.033 | |
| 209 | 0.354 | |
| 234 | 11 | |
| 284 | 1.95 | |
| 285 | 0.102 | |
| 286 | 0.079 | |
| 287 | 0.041 | |
| 288 | 0.104 | |
| 289 | 1.3 | |
| 291 | 5.1 | |
| 294 | 2.1 | |
| 295 | 1.2 | |
| 296 | 0.284 | |
| 297 | 0.34 | |
| 298 | 0.025 | |
| 299 | 2.3 | |
| 300 | 0.251 | |
| 301 | 0.63 | |
| 302 | 0.077 | |

### Human Peripheral Blood Mononuclear Leukocyte Cell Assay.

Human peripheral blood mononuclear leukocytes are challenged with 25 ng/mL lipopolysaccharide (LPS) in the absence or presence of Test Compound and incubated for 16 hours as described by Welker P. et al, International Archives Allergy and Immunology (1996) 109: 110. The quantity of LPS-induced tumor necrosis factor-alpha (TNF-alpha) cytokine release is measured by a commercially available Enzyme-Linked Immunosorbent Assay (ELISA) kit. Test compounds are evaluated for their ability to inhibit TNF-alpha release. Table 2 records IC₅₀ values for inhibition of TNF-alpha release by Test Compounds of the present invention, wherein the IC₅₀ value, in micromolar concentration, represents the concentration of Test Compound resulting in a 50% inhibition of TNF-alpha release from human peripheral blood mononuclear leukocytes as compared to control experiments containing no Test Compound.

TABLE 4

| Example Number | IC50, uM |
|---|---|
| 3 | 6.1 |
| 13 | 6.32 |
| 21 | 3.4 |
| 29 | 2.68 |
| 31 | 4.52 |
| 60 | 2.34 |
| 296 | 3.49 |
| 300 | 4.78 |
| 302 | 5.45 |

TABLE 5

| Example | uM |
|---|---|
| 303 | 0.089 |
| 306 | 0.058 |
| 307 | 0.049 |
| 308 | 0.12 |
| 309 | 0.30 |
| 310 | 0.13 |
| 311 | 0.182 |
| 312 | 0.349 |
| 313 | 0.25 |
| 314 | 0.25 |
| 315 | 54% @ 10 |
| 316 | 0.42 |
| 317 | 0.068 |
| 318 | 0.67 |
| 319 | 0.32 |
| 320 | 0.79 |
| 321 | 0.52 |
| 322 | 2.02 |
| 323 | 51% @ 10, 9% @ 1 |
| 324 | 40% @ 10 |
| 325 | 54% @ 10 |
| 326 | 41% @ 10 |
| 327 | 0.81 |
| 328 | 68% @ 10 |
| 329 | 0.25 |
| 330 | 2.1 |
| 331 | 71% @ 10 |
| 332 | 0.05 |
| 333 | 2.438 |
| 334 | 2.2 |
| 335 | 0.014 |
| 336 | 27% @ 10 |
| 337 | 8% @ 10 |
| 338 | 7% @ 2 |
| 339 | 9% @ 2 |
| 340 | 31% @ 10 |
| 342 | 1 |
| 344 | 0.18 |
| 345 | 0.27 |
| 346 | 19% @0.1, 63% @1 |
| 347 | 8% @0.1, 31% @1 |
| 348 | 17% @0.1, 47% @1 |
| 350 | 39% @0.1, 64% @1 |
| 352 | 0.043 |
| 354 | 0.11 |
| 355 | 0.042 |
| 356 | 0.059 |
| 357 | 0.13 |
| 358 | 0.1 |
| 359 | 0.013 |
| 360 | 0.39 |
| 361 | 0.87 |
| 363 | 0.19 |
| 364 | 0.01 |
| 365 | 0.007 |
| 366 | 53% @ 10 |
| 367 | 85% @ 10 |
| 369 | 0.00 |
| 372 | 1.46 |
| 374 | 0.037 |
| 376 | 0.48 |
| 378 | 0.400 |
| 380 | 2.00 |
| 382 | 0.88 |
| 383 | 0.1 |
| 384 | 96% @ 10, 72% @ 1 |
| 385 | 0.03 |
| 386 | 0.1138 |
| 387 | 4.446 |
| 388 | 1.645 |
| 389 | 0.09 |
| 390 | 24% @0.1, 63% @1 |
| 391 | 0.91 |
| 392 | 75% @0.1, 86% @1 |
| 393 | 0.4 |
| 395 | 0.056 |
| 396 | 3% @0.1, 56% @1 |
| 397 | 0.04 |
| 399 | 21% @0.1, 74% @1 |
| 404 | −11% @0.1, 48% @1 |
| 406 | 0.01343 |
| 407 | 0.01032 |
| 408 | 0.1165 |
| 409 | 0.03089 |
| 411 | 0.008 |
| 532 | 66% @ 10 |
| 415 | 28% @0.1, 69% @1 |
| 416 | 67% @0.1, 83% @1 |
| 418 | 0.075 |
| 420 | 0.0147 |
| 421 | 0.0147 |
| 426 | 0.013 |
| 427 | 0.819 |
| 428 | 0.1403 |
| 429 | 1.068 |
| 430 | 0.9424 |
| 432 | 24% @ 10, 9% @ 1 |
| 434 | 1.9 |
| 436 | 16% @0.1, 41% @1 |
| 438 | 1.3 |
| 440 | 0.92 |
| 442 | 3.01 |
| 444 | 5.00 |
| 450 | 192% @ 10, 89% @ 1 |
| 451 | 3% @1 |
| 455 | 0.10 |
| 456 | 0.04 |
| 457 | 1.1 |
| 458 | 1.0 |
| 459 | 0.02 |
| 461 | 0.017 |
| 463 | 0.056 |
| 464 | 0.019 |
| 465 | 0.24 |
| 466 | 0.007 |
| 467 | 0.119 |
| 468 | 0.0086 |
| 470 | 0.005 |
| 472 | 0.050 |
| 473 | 0.004 |
| 474 | 0.38 |
| 475 | 0.01 |
| 476 | 0.024 |
| 477 | 0.042 |
| 480 | 0.15 |
| 481 | 0.23 |
| 482 | 0.084 |
| 483 | 0.27 |
| 484 | 0.019 |
| 485 | 0.050 |
| 486 | 0.057 |
| 487 | 0.038 |
| 488 | 0.54 |
| 489 | 0.52 |
| 490 | 0.34 |
| 491 | 0.34 |
| 492 | 0.04 |
| 493 | 73% @ 10, 36% @ 1 |
| 494 | 0.67 |
| 495 | 0.25 |
| 498 | 25% @1 |
| 499 | 78% @ 0.1, 95% @1 |
| 506 | 0.042 |
| 507 | 0.007 |
| 508 | 0.052 |
| 509 | 0.5 |
| 510 | 0.47 |
| 511 | 0.024 |
| 512 | 0.032 |
| 513 | 0.41 |
| 514 | 0.57 |
| 516 | 0.45 |
| 519 | 0.052 |
| 520 | 0.0092 |
| 522 | 0.78 |
| 525 | 11% @ 1 |
| 526 | 25% @ 0.1, 70% @1 |
| 527 | 0.0033 |
| 528 | 35% @ 1 |
| 529 | 40% @ 10 |
| 530 | 83% @ 10 |
| 531 | 14% @ 10 |
| 533 | 39% @ 10 |

### Abl Kinase Assay

### Assay A1

The activity of Abl kinase was determined by following the production of ADP from the kinase reaction through coupling with the pyruvate kinase/lactate dehydrogenase system (e.g., Schindler, et al. Science (2000) 289, 1938-1942). In this assay, the oxidation of NADH (thus the decrease at A340 nm) was continuously monitored spectrophotometrically. The reaction mixture (100 μl) contained Abl kinase (1.9 nM, nominal concentration), peptide substrate (EAIYAAPFAKKK, 0.2 mM), pyruvate kinase (3.5 units), lactate dehydrogenase (5.5 units), phosphoenolpyruvate (1 mM), and NADH (0.28 mM) in 60 mM Tris buffer containing 0.13% octyl-glucoside, 13 mM MgCl₂ and 3.5% DMSO at pH 7.5. The reaction was initiated by adding ATP (0.2 mM, final concentration). The absorption at 340 nm was continuously monitored for 3 h at 30° C. on a Polarstar Optima plate reader (BMG). The reaction rate was calculated using the 1 h to 2 h time frame. Percent inhibition was obtained by comparison of reaction rate with that of a control (i.e. with no test compound). IC₅₀ values were calculated from a series of percent inhibition values determined at a range of inhibitor concentrations using software routines as implemented in the GraphPad Prism software package.

### Assay A2

Abl kinase assay A2 is the same as for assay A1 except that (1) a nominal concentration of 1.1 nM of enzyme was employed (2) the reaction was pre-incubated at 30° C. for 2 h prior to initiation with ATP (3) 0.5 mM ATP (final concentration) was used to initiate the reaction.

Ab1 protein sequence used for screening:
SPNYDKWEMERTDITMKHKLGGGQYGEVYEGVWKKYSLTVAVKTLKEDTM
EVEEFLKEAAVMKEIKHPNLVQLLGVCTREPPFYIITEFMTYGNLLDYLR
ECNRQEVNAVVLLYMATQISSAMEYLEKKNFIHRDLAARNCLVGENHLVK
VADFGLSRLMTGDTYTAHAGAKFPIKWTAPESLAYNKFSIKSDVWAFGVL
LWEIATYGMSPYPGIDLSQVYELLEKDYRMERPEGCPEKVYELMRACWQW
NPSDRPSFAEIHQAFETMFQESSISDEVEKELGK

### KDR Kinase Assay

### Assay K1

The activity of KDR kinase was determined by following the production of ADP from the kinase reaction through coupling with the pyruvate kinase/lactate dehydrogenase system (e.g., Schindler, et al. Science (2000) 289, 1938-1942). In this assay, the oxidation of NADH (thus the decrease at A₃₄₀ₙₘ) was continuously monitored spectrophotometrically. The reaction mixture (100 μl) contained KDR (1.5 nM to 7.1 nM, nominal concentration), polyE₄Y (1 mg/ml), pyruvate kinase (3.5 units), lactate dehydrogenase (5.5 units), phosphoenolpyruvate (1 mM), and NADH (0.28 mM) in 60 mM Tris buffer containing 0.13% octyl-glucoside, 13 mM MgCl₂, 6.8 mM DTT, and 3.5% DMSO at pH 7.5. The reaction was initiated by adding ATP (0.2 mM, final concentration). The absorption at 340 nm was continuously monitored for 3 h at 30° C. on a Polarstar Optima plate reader (BMG). The reaction rate was calculated using the 1 h to 2 h time frame. Percent inhibition was obtained by comparison of reaction rate with that of a control (i.e. with no test compound). IC₅₀ values were calculated from a series of percent inhibition values determined at a range of inhibitor concentrations using software routines as implemented in the GraphPad Prism software package.

### Assay K2

KDR kinase assay K2 is the same as for assay K1 except that (1) a nominal concentration of 2.1 DM of enzyme was employed (2) the reaction was pre-incubated at 30° C. for 2 h prior to initiation with ATP (3) 1.0 mM ATP (final concentration) was used to initiate the reaction.

KDR protein sequence used for screening:
DPDELPLDEHCERLPYDASKWEFPRDRLKLGKPLGRGAFGQVIEADAFGI
DKTATCRTVAVKMLKEGATHSEHRALMSELKILIHIGHHLNVVNLLGACT
KPGGPLMVIVEFCKFGNLSTYLRSKRNEFVPYKVAPEDLYKDFLTLEHLI
CYSFQVAKGMEFLASRKCIHRDLAARNILLSEKNVVKICDFGLARDIYKD
PDYVRKGDARLPLKWMAPETIFDRVYTIQSDVWSFGVLLWEIFSLGASPY
PGVKIDEEFCRRLKEGTRMRAPDYTTPEMYQTMLDCWHGEPSQRPTFSEL
VEHLGNLLQANAQQD

### B-Raf(V599E) Kinase Assay

### Assay B1

The activity of B-Raf(V599E) kinase was determined by following the formation of ADP from the reaction through coupling with the pyruvate kinase/lactate dehydrogenase system (e.g., Schindler, et al. Science (2000) 289, 1938-1942). In this assay, the oxidation of NADH (thus the decrease at A₃₄₀ₙₘ) was continuously monitored spectrophotometrically. The reaction mixture (100 μl) contained B-Raf(V599E) kinase (0.34 nM nominal concentration, construct 1), unphosphorylated, full-length MEK1 (42 nM), MgCl₂ (13 mM), pyruvate kinase (3.5 units), lactate dehydrogenase (5.5 units), phosphoenolpyruvate (1 mM), and NADH (0.28 mM), in 60 mM Tris buffer, containing 0.13% octyl-glucoside and 3.5% DMSO concentration at pH 7.5. The test compounds were incubated with the reaction mixture at 30° C. for 2 h. The reaction was initiated by adding ATP (0.2 mM, final concentration). The absorption at 340 nm was continuously monitored for 3 h at 30° C. on a Polarstar Optima plate reader (BMG). The reaction rate was calculated using the 1.5 h to 2.5 h time frame. Percent inhibition was obtained by comparison of reaction rate with that of a control (i.e. with no test compound). IC₅₀ values were calculated from a series of percent inhibition values determined at a range of inhibitor concentrations using software routines as implemented in the GraphPad Prism software package.

### Assay B2

Same as assay B1 except that (1) construct 2 was employed at a nominal concentration of 2 nM (2) the reaction was pre-incubated at 30° C. for 1 h prior to initiation with ATP (3) a reading time frame of 0.5 h to 1.5 h.

B-Raf(V599E) construct 1 protein sequence used for
screening:
KSPGQRERKSSSSSEDRNRMKTLGRRDSSDDWEIPDGQITVGQRIGSGSF
GTVYKGKWHGDVAVKMLNVTAPTPQQLQAFKNEVGVLRKTRHVNILLFMG
YSTKPQLAIVTQWCEGSSLYHHLHIIETKFEMIKLIDIARQTAQGMDYLH
AKSIIHRDLKSNNIFLHEDLTVKIGDFGLATEKSRWSGSHQFEQLSGSIL
WMAPEVIRMQDKNPYSFQSDVYAFGIVLYELMTGQLPYSNINNRDQIIFM
VGRGYLSPDLSKVRSNCPKAMKRLMAECLKKKRDERPLFPQILASIELLA
RSLPKIHRSASEPSLNRAGFQTEDFSLYACASPKTPIQAGGYGAFPVH
B-Raf(V599E) construct 2 protein sequence used for
screening:
EDRNRMKTLGRRDSSDDWEIPDGQITVGQRIGSGSFGTVYKGKWHGDVAV
KMLNVTAPTPQQLQAFKNEVGVLRKTRHVNILLFMGYSTKPQLAIVTQWC
EGSSLYHHLHIIETKFEMIKLIDIARQTAQGMDYLHAKSIIHRDLKSNNI
FLHEDLTVKIGDFGLATEKSRWSGSHQFEQLSGSILWMAPEVIRMQDKNP
YSFQSDVYAFGIVLYELMTGQLPYSNINNRDQIIFMVGRGYLSPDLSKVR
SNCPKAMKRLMAECLKKKRDERPLFPQILASIELLARSLPKIHR
MEK1 protein sequence used for screening:
MELKDDDFEKISELGAGNGGVVFKVSHKPSGLVMARKLIHLEIKPAIRNQ
IIRELQVLHECNSPYIVGFYGAFYSDGEISICMEHMDGGSLDQVLKKAGR
IPEQILGKVSIAVIKGLTYLREKHKIMHRDVKPSNILVNSRGEIKLCDFG
VSGQLIDSMANSFVGTRSYMSPERLQGTHYSVQSDIWSMGLSLVEMAVGR
YPIPPPDAKELELMFGCQVEGDAAETPPRPRTPGRPLSSYGMDSRPPMAI
FELLDYIVNEPPPKLPSGVFSLEFQDFVNKCLIKNPAERADLKQLMVHAF
IKRSDAEEVDFAGWLCSTIGLNQPSTPTHAAGV

### P-38 alpha Kinase Assay

### Assay P1

The activity of phosphorylated p-38-alpha kinase was determined by following the formation of ADP from the kinase reaction through coupling with the pyruvate kinase/lactate dehydrogenase system (e.g., Schindler, et al. Science (2000) 289, 1938-1942). In this assay, the oxidation of NADH (thus the decrease at A340 nm) was continuously measured spectrophotometrically. The reaction mixture (100 μl) contained phosphorylated p-38 alpha kinase (7.1-9 nM nominal concentration), peptide substrate (IPTSPITTFYFFFKKK-OH, 0.2 mM), MgC₂ (13 mM), pyruvate kinase (3.5 units), lactate dehydrogenase (5.5 units), phosphoenolpyruvate (1 mM), and NADH (0.28 mM) in 60 mM Tris buffer at pH 7.5, containing 130 uM n-Dodecyl-B-D-maltopyranoside and 3.5% DMSO concentration. The test compounds were incubated with the reaction mixture at 30° C. for 2 h before the addition of ATP (0.3 mM final concentration). The absorption at 340 nm was monitored continuously for up to 3 h at 30° C. on Polarstar Optima plate reader (BMG). The reaction rate was calculated using the time frame from 1.5 h to 2.5 h. Percent inhibition was obtained by comparison of reaction rate with that of a control (i.e. with no test compound). IC₅₀ values were calculated from a series of percent inhibition values determined at a range of inhibitor concentrations using software routines as implemented in the GraphPad Prism software package.

### Assay P2

Same as assay P1 except that (1) the reaction was not pre-incubated.

P38-alpha protein sequence used for screening:
MSQERPTFYRQELNKTIWEVPERYQNLSPVGSGAYGSVCAAFDTKTGLRV
AVKKLSRPFQSIIHAKRTYRELRLLKHMKHENVIGLLDVFTPARSLEEFN
DVYLVTHLMGADLNNIVKCQKLTDDHVQFLIYQILRGLKYIHSADIIHRD
LKPSNLAVNEDCELKILDFGLARHTDDEMTGYVATRWYRAPEIMLNWMHY
NQTVDIWSVGCIMAELLTGRTLFPGTDHINQLQQIMRLTGTPPAYLINRM
PSHEARNYIQSLTQMPKMNFANVFIGANPLAVDLLEKMLVLDSDKRITAA
QALAHAYFAQYHDPDDEPVADPYDQSFESRDLLIDEWKSLTYDEVISFVP
PPLDQEEMES

P38-alpha Assay data

| Example | Results, μM (IC50) | Method |
|---|---|---|
| 430 | 0.006 | P1 |
| 431 | 0.028 | P1 |
| 432 | 0.011 | P1 |
| 433 | 0.007 | P1 |
| 434 | 0.004 | P1 |
| 435 | 0.006 | P1 |
| 436 | 0.007 | P1 |
| 437 | 0.029 | P1 |
| 438 | 0.010 | P1 |
| 439 | 0.013 | P1 |
| 440 | 0.009 | P1 |
| 441 | 0.005 | P1 |
| 442 | 0.005 | P1 |
| 443 | 0.030 | P1 |
| 444 | 0.006 | P1 |
| 445 | 0.010 | P1 |
| 446 | 0.005 | P1 |
| 447 | 0.011 | P1 |
| 448 | 0.027 | P1 |
| 449 | 0.022 | P1 |
| 450 | 0.013 | P1 |
| 451 | 0.008 | P1 |
| 452 | 0.006 | P1 |
| 453 | 0.040 | P1 |
| 454 | 0.014 | P1 |
| 455 | 0.018 | P1 |
| 456 | 0.015 | P1 |
| 457 | 0.008 | P1 |
| 458 | 0.007 | P1 |
| 459 | 0.005 | P1 |
| 460 | 0.008 | P1 |
| 461 | 0.030 | P1 |
| 462 | 0.008 | P1 |
| 463 | 0.025 | P1 |
| 464 | 0.011 | P1 |
| 465 | 0.013 | P1 |
| 466 | 0.038 | P1 |
| 467 | 0.011 | P1 |
| 468 | 0.009 | P1 |
| 469 | 0.009 | P1 |
| 470 | 0.025 | P1 |
| 471 | 0.016 | P1 |
| 472 | 0.010 | P1 |
| 473 | 0.030 | P1 |
| 474 | 0.013 | P1 |
| 475 | 0.037 | P1 |
| 476 | 0.006 | P1 |
| 477 | 0.038 | P1 |
| 478 | 0.006 | P1 |
| 479 | 0.037 | P1 |
| 480 | 0.007 | P1 |
| 481 | 0.007 | P1 |
| 482 | 0.010 | P1 |
| 483 | 0.027 | P1 |
| 484 | 0.009 | P1 |
| 485 | 0.012 | P1 |
| 486 | 0.005 | P1 |
| 487 | 0.006 | P1 |
| 488 | 0.007 | P1 |
| 489 | 0.066 | P1 |
| 490 | 0.031 | P1 |
| 491 | 0.013 | P1 |
| 492 | 0.008 | P1 |
| 493 | 0.042 | P1 |
| 494 | 0.068 | P1 |
| 495 | 0.038 | P1 |
| 496 | 0.031 | P1 |
| 497 | 0.018 | P1 |
| 498 | 0.041 | P1 |
| 499 | 0.046 | P1 |
| 500 | 0.017 | P1 |
| 501 | 0.076 | P1 |
| 502 | 0.034 | P1 |
| 503 | 1.5 | P1 |
| 504 | 0.082 | P1 |
| 506 | 0.009 | P1 |
| 507 | 0.18 | P1 |
| 508 | 0.33 | P1 |
| 509 | 57% @1.0 | P1 |
| 510 | 0.12 | P1 |
| 511 | 0.30 | P1 |
| 512 | 82% @1.0 | P1 |
| 513 | 0.012 | P1 |
| 514 | 0.011 | P1 |
| 515 | 90% @1.0 | P1 |
| 516 | 0.015 | P1 |
| 517 | 0.025 | P1 |

KDR Assay Data

| Example | Results, μM (IC50) | Method |
|---|---|---|
| 433 | 63% @10 | K1 |
| 434 | 2.9 | K1 |
| 437 | 32% @10 | K1 |
| 438 | 39% @10 | K1 |
| 440 | 56% @10 | K1 |
| 441 | 62% @10 | K1 |
| 442 | 25% @10 | K1 |
| 443 | 33% @10 | K1 |
| 446 | 1.5 | K1 |
| 449 | 33% @10 | K1 |
| 450 | 45% @10 | K1 |
| 453 | 34% @10 | K1 |
| 454 | 27% @10 | K1 |
| 456 | 53% @10 | K1 |
| 457 | 31% @10 | K1 |
| 459 | 33% @10 | K1 |
| 460 | 26% @10 | K1 |
| 461 | 33% @10 | K1 |
| 462 | 25% @10 | K1 |
| 463 | 47% @10 | K1 |
| 464 | 85% @10 | K1 |
| 465 | 91% @10 | K1 |
| 466 | 50% @10 | K1 |
| 467 | 2.9 | K1 |
| 476 | 1.1 | K1 |
| 478 | 99% @10 | K1 |
| 480 | 92% @10 | K1 |
| 481 | 102% @10 | K1 |
| 482 | 1.4 | K1 |
| 483 | 0.21 | K1 |
| 484 | 37% @10 | K1 |
| 485 | 79% @10 | K1 |
| 487 | 20% @1.0 | K2 |
| 488 | 41% @1.0 | K2 |
| 489 | 25% @1 | K2 |
| 491 | 77% @10 | K1 |
| 492 | 50% @10 | K1 |
| 493 | 82% @10 | K1 |
| 494 | 82% @10 | K1 |
| 495 | 2.7 | K2 |
| 496 | 2.4 | K1 |
| 497 | 43% @10 | K1 |
| 498 | 60% @10 | K1 |
| 499 | 0.40 | K1 |
| 500 | 0.009 | K2 |
| 501 | 1.6 | K1 |
| 502 | 1.3 | K2 |
| 503 | 0.19 | K2 |
| 504 | 0.049 | K1 |
| 506 | 0.021 | K1 |
| 510 | 0.007 | K1 |
| 511 | 0.014 | K2 |
| 512 | 0.029 | K2 |
| 514 | 0.033 | K2 |

BRaf Assay Data

| Number | Results, μM (IC50) | Method |
|---|---|---|
| 433 | 37% @ 1.0 | B1 |
| 434 | 0.006 | B1 |
| 435 | 0.16 | B1 |
| 436 | 3.7 | B2 |
| 437 | 0.16 | B1 |
| 441 | 0.037 | B1 |
| 442 | 24% @ 1.0 | B1 |
| 443 | 20% @ 1.0 | B1 |
| 444 | 42% @ 1.0 | B1 |
| 445 | 39% @ 1.0 | B1 |
| 446 | 0.016 | B1 |
| 451 | 0.84 | B1 |
| 452 | 45% @ 1.0 | B1 |
| 455 | 30% @ 1.0 | B1 |
| 458 | 28% @ 1.0 | B1 |
| 459 | 5% @ 10 | B2 |
| 460 | 2.4 | B1 |
| 461 | 28% @ 10 | B1 |
| 462 | 43% @ 1.0 | B1 |
| 463 | 23% @ 1.0 | B1 |
| 464 | 0.043 | B1 |
| 465 | 0.011 | B1 |
| 466 | 24% @ 1.0 | B1 |
| 467 | 0.032 | B1 |
| 468 | 1.2 | B1 |
| 469 | 43% @ 1.0 | B1 |
| 471 | 39% @ 10 | B2 |
| 473 | 4% @ 10 | B2 |
| 476 | 0.041 | B1 |
| 478 | 0.028 | B1 |
| 480 | 0.029 | B1 |
| 481 | 0.041 | B1 |
| 482 | 0.0038 | B1 |
| 483 | 0.014 | B1 |
| 484 | 7.4 | B1 |
| 488 | 21% @ 1.0 | B2 |
| 491 | 0.007 | B1 |
| 492 | 0.028 | B1 |
| 494 | 0.007 | B1 |
| 495 | 0.009 | B1 |
| 496 | 0.010 | B1 |
| 497 | 0.045 | B1 |
| 498 | 0.074 | B1 |
| 499 | 0.026 | B1 |
| 500 | 0.010 | B1 |
| 503 | 0.13 | B1 |
| 504 | 0.043 | B1 |
| 506 | 0.020 | B1 |
| 507 | 0.035 | B1 |
| 508 | 0.020 | B1 |
| 509 | 0.28 | B1 |
| 510 | 0.21 | B1 |
| 511 | 1.7 | B1 |
| 513 | 0.008 | B1 |
| 514 | 0.036 | B2 |
| 515 | 27% @ 1.0 | B2 |
| 516 | 0.022 | B1 |
| 517 | 0.075 | B2 |

Abl Assay Data

| Example | Results, μM (IC50) | Method |
|---|---|---|
| 431 | 29% @10 | A1 |
| 433 | 39% @10 | A1 |
| 434 | 3.9 | A1 |
| 437 | 21% @10 | A1 |
| 441 | 44% @10 | A1 |
| 445 | 30% @10 | A1 |
| 446 | 4.8 | A2 |
| 452 | 37% @10 | A1 |
| 463 | 44% @10 | A1 |
| 464 | 82% @10 | A1 |
| 465 | 1.2 | A1 |
| 466 | 1.2 | A1 |
| 467 | 4.8 | A1 |
| 469 | 22% @10 | A1 |
| 470 | 15% @10 | A1 |
| 471 | 14% @1 | A1 |
| 476 | 8.4 | A1 |
| 478 | 91% @10 | A1 |
| 480 | 94% @10 | A1 |
| 481 | 93% @10 | A1 |
| 482 | 3.2 | A1 |
| 483 | 11.3 | A1 |
| 491 | 78% @10 | A2 |
| 492 | 49% @10 | A1 |
| 493 | 17% @1 | A1 |
| 494 | 96% @10 | A1 |
| 495 | 7.6 | A1 |
| 496 | 66% @10 | A1 |
| 497 | 30% @10 | A1 |
| 498 | 48% @10 | A1 |
| 499 | 88% @10 | A1 |
| 500 | 0.021 | A2 |
| 501 | 2.5 | A1 |
| 502 | 3.1 | A1 |
| 503 | 0.29 | A2 |
| 504 | 0.063 | A1 |
| 506 | 0.0066 | A2 |
| 507 | 23.3 | A1 |
| 508 | 32% @10 | A1 |
| 510 | 0.062 | A2 |
| 511 | 0.25 | A2 |
| 512 | 0.11 | A2 |
| 513 | 0.023 | A2 |
| 514 | 0.077 | A2 |
| 516 | 0.0018 | A2 |
| 517 | 0.0022 | A2 |

We claim:

1. Compounds of Formula IA 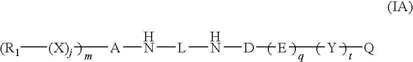
wherein:
R₁ is selected from the group consisting of aryls, heteroaryls, and heterocyclyls;
each X and Y is individually selected from the group consisting of —O—, —S—, —NR₆—, —NR₆SO₂—, —NR₆CO—, alkynyls, alkenyls, alkylenes, —O(CH₂)ₕ—, and —NR₆(CH₂)ₕ—, where each h is individually selected from the group consisting of 1, 2, 3, or 4, and where for each of alkylenes (preferably C₁-C₁₈, and more preferably C₁-C₁₂), —O(CH₂)ₕ—, and —NR₆(CH₂)ₕ—, one of the methylene groups present therein may be optionally double-bonded to a side-chain oxo group except that where —O(CH₂)ₕ— the introduction of the side-chain oxo group does not form an ester moiety;
A is selected from the group consisting of aromatic, monocycloheterocyclic, and bicycloheterocyclic rings;
D is phenyl or a five- or six-membered heterocyclic ring selected from the group consisting of pyrazolyl, pyrrolyl, imidazolyl, oxazolyl, thiazolyl, furyl, oxadiazolyl, thiadiazolyl, thienyl, pyridyl, and pyrimidyl;
E is selected from the group consisting of phenyl, pyridinyl, and pyrimidinyl;
L is selected from the group consisting of —C(O)— and —S(O)₂—;
j is 0 or 1;
k is 0 or 1;
m is 0 or 1;
n is 0 or 1;
q is 0 or 1;
t is 0 or 1;
u is 1, 2, 3, or 4;
v is 1, 2, or 3;
x is 1 or 2;
Q is selected from the group consisting of 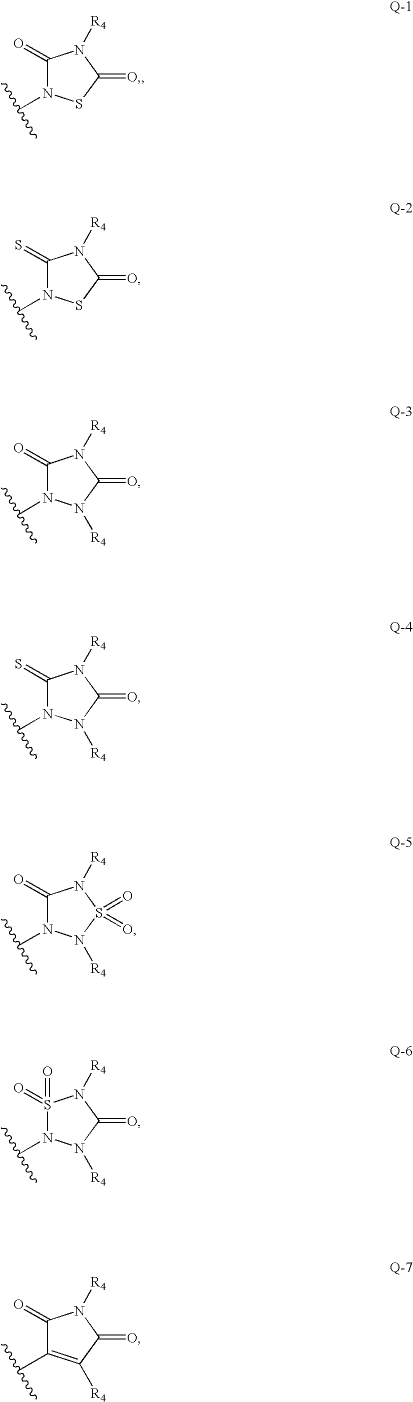 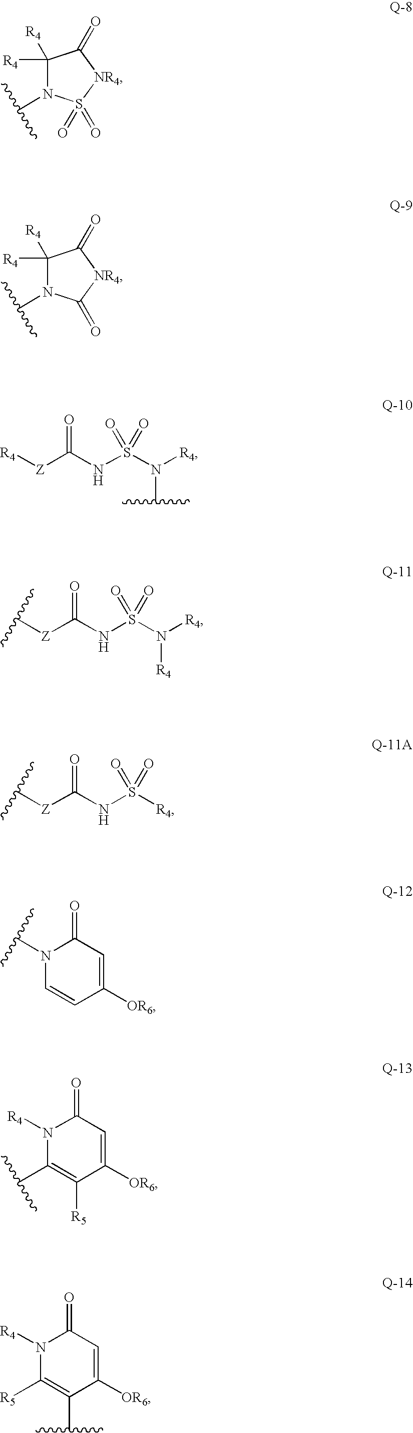 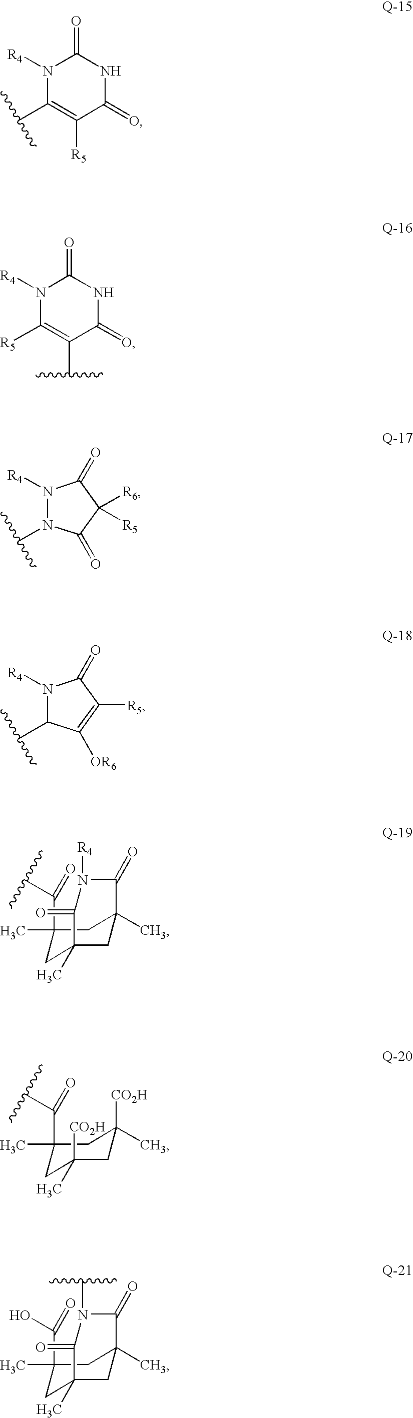 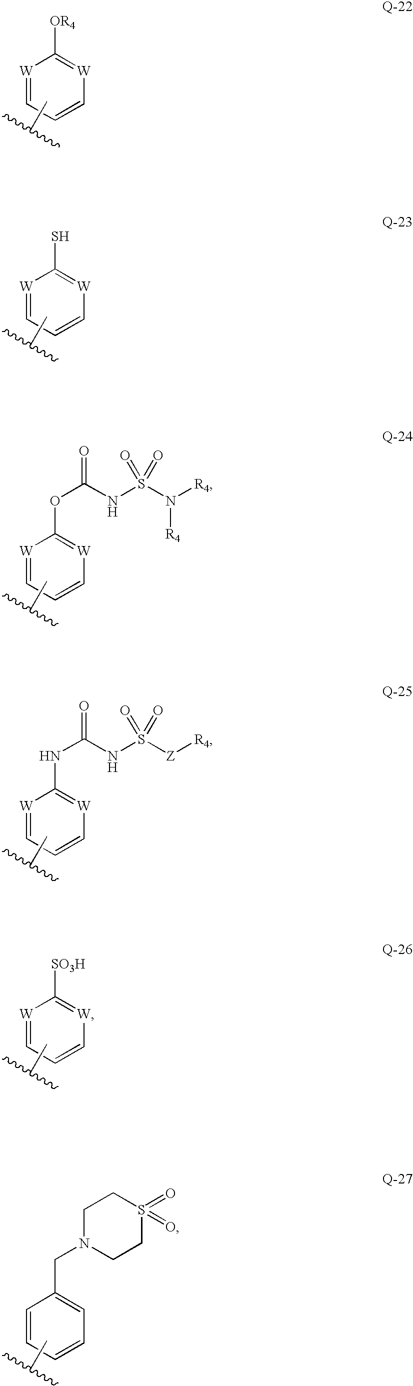 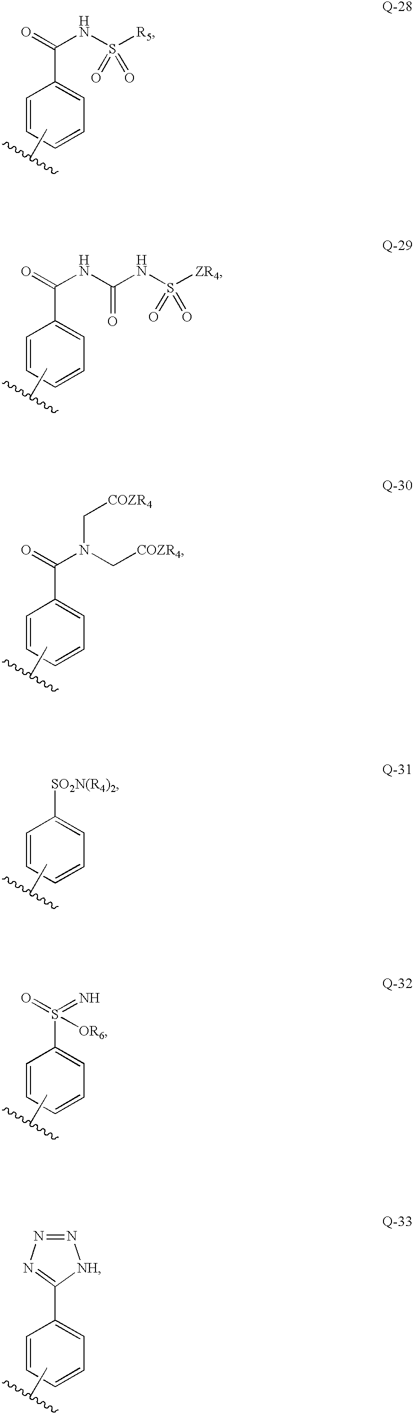 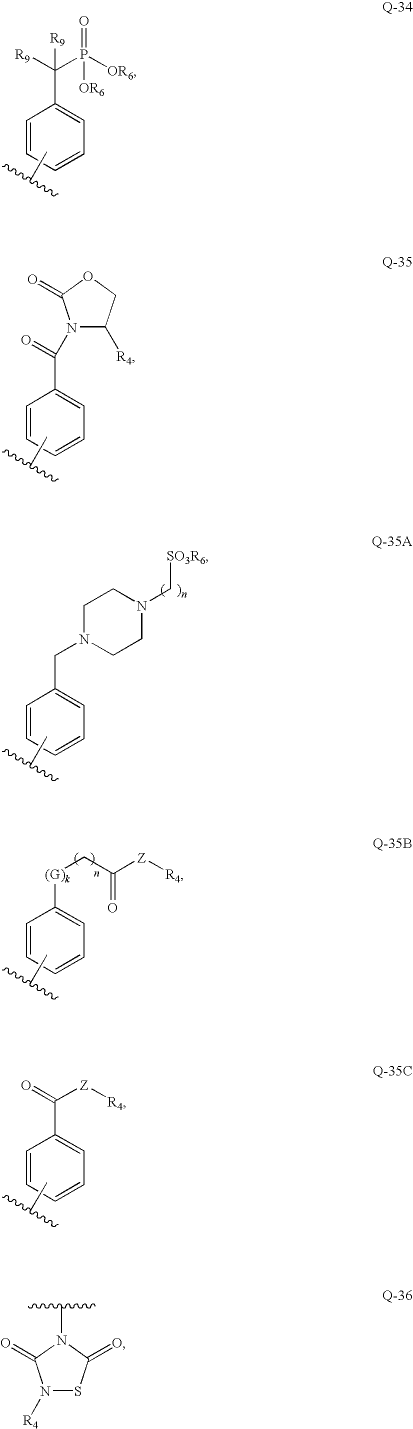 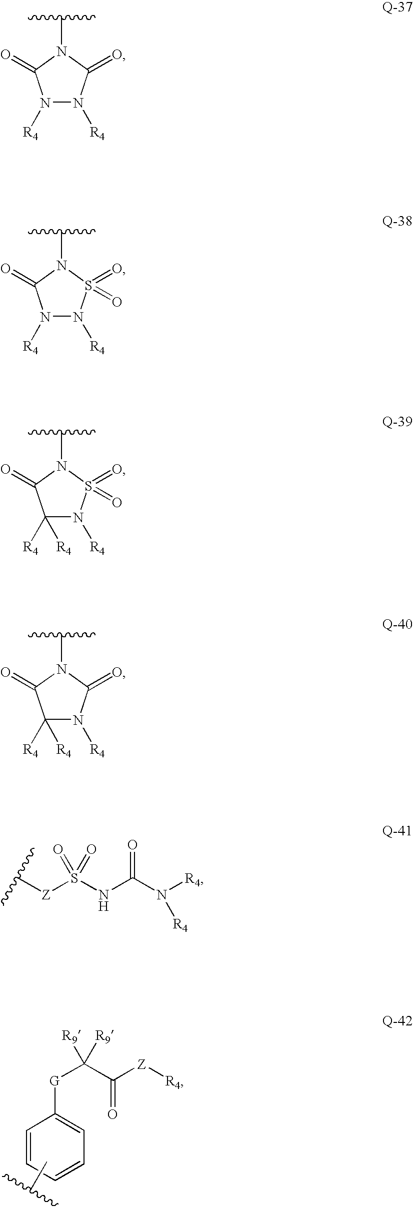 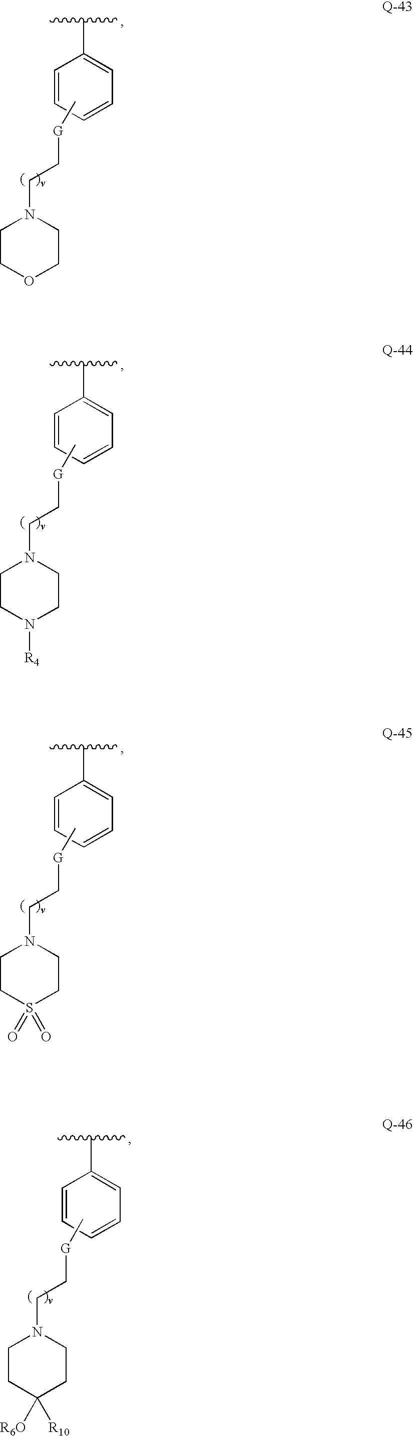 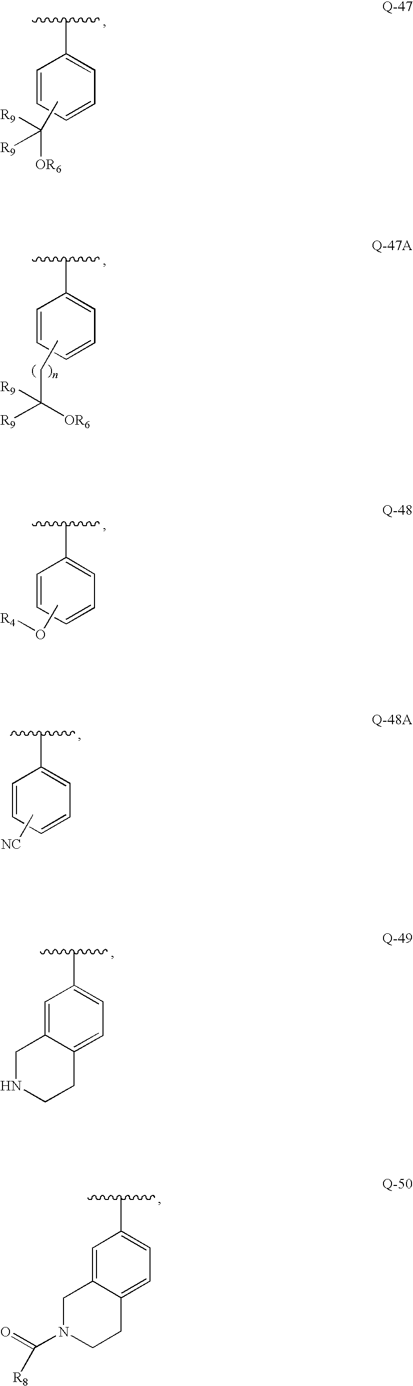 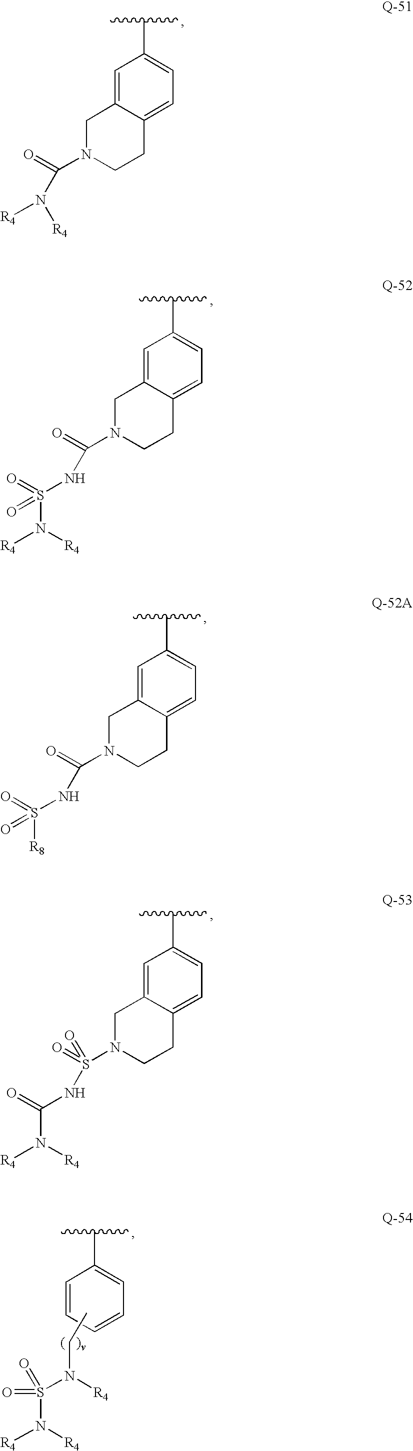 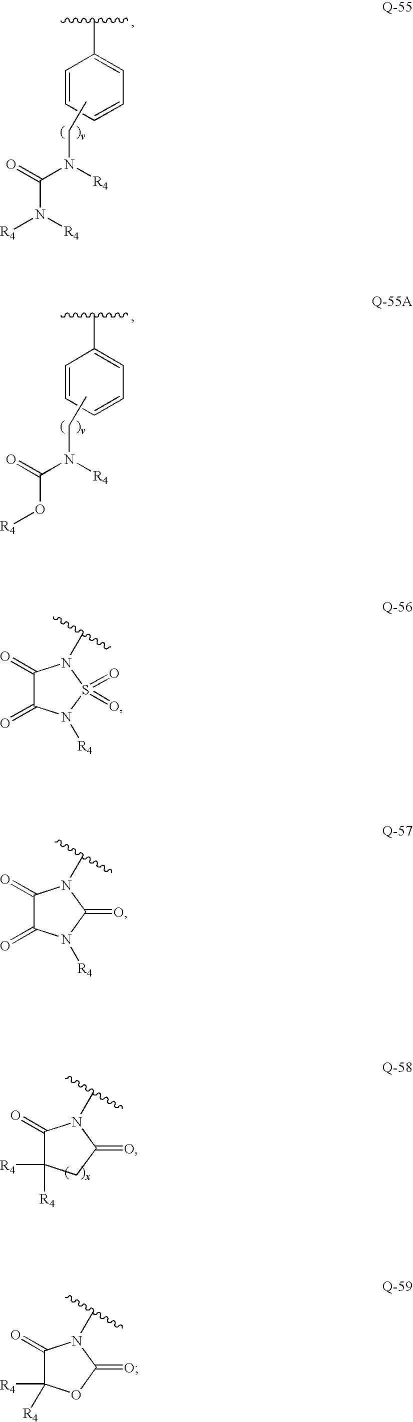
each R₄ group is individually selected from the group consisting of —H, alkyls wherein one or more carbon atoms are optionally substituted with hydroxyl moieties, branched alkyls wherein one or more carbon atoms are optionally substituted with hydroxyl moieties, aminoalkyls, alkoxyalkyls, aryls, aralkyls, heterocyclyls, and heterocyclylalkyls except when the R₄ constituent places a heteroatom on an alpha-carbon directly attached to a ring nitrogen on Q;
when two R₄ groups are bonded with the same atom, the two R₄ groups optionally form an alicyclic or heterocyclic 4-7 membered ring;
each R₅ is individually selected from the group consisting of —H, alkyls, aryls, heterocyclyls, alkylaminos, arylaminos, cycloalkylaminos, heterocyclylaminos, hydroxys, alkoxys, aryloxys, alkylthios, arylthios, cyanos, halogens, perfluoroalkyls, alkylcarbonyls, and nitros;
each R₆ is individually selected from the group consisting of —H, alkyls, alkyls, and β-trimethylsilylethyl;
each R₈ is individually selected from the group consisting of alkyl, wherein one or more carbon atoms can be optionally substituted with a hydroxyl moiety, branched alkylC₄-C₇, wherein one or more carbon atoms can be optionally substituted with a hydroxyl moiety, phenyl, naphthyl, aralkyls, heterocyclyls, and heterocyclylalkyls;
each R₉ group is individually selected from the group consisting of —H, —F, alkylnylC2-C5, alkyls, and perfluoroalkylC₁-C₃ wherein when two R₉ groups are geminal alkyl groups, said geminal alkyl groups may be cyclized to form a 3-6 membered ring;
each R₉ group is independently and individually selected from the group consisting of —H, —F, alkyl(C₁-C₆), and perfluoroalkylC₁-C₃ wherein when two R₉ groups are geminal alkyl groups, said geminal alkyl groups may be cyclized to form a 3-6 membered ring;
each R₁₀ is alkyl or fluoroalkyl wherein the fluoroalkyl moiety is partially or fully fluorinated;
G is alkylene, N(R₄), O;
W is CH or N;
each Z is individually selected from the group consisting of —O— and —N(R₄)—; and
each ring of formula (IA) optionally includes one or more of R₇, where R₇ is a noninterfering substituent individually selected from the group consisting of —H, alkyl, aryl, heterocyclyl, alkylamino, arylamino, cycloalkylamino, heterocyclylamino, hydroxy, alkoxy, aryloxy, alkylthio, arthylthio, cyano, halogen, nitro, alkylsulfinyl, alkylsulfonyl, aminosulfonyl, alkylaminosulfonyl, dialkylaminosulfonyl, aminocarbonyl, alkylaminocarbonyl, dialkylaminocarbonyl, carbonylamino, carbonylNH(alkyl), carbonylN(alkyl)₂, and perfluoroalkyl, wherein the aryl or heterocyclyl ring may optionally be further substituted by halogen, cyano, or C1-C3 alkyl;
except that:
when Q is Q-7, q is 0, and R₅ and D are phenyl, then A is not phenyl, oxazolyl, pyridyl, pyrimidyl, pyrazolyl, or imidazolyl;
when Q is Q-8, then Y is not —CH₂O—;
when Q is Q-10, t is 0, and E is phenyl, then any R₇ on E is not an o-alkoxy;
when Q is Q-11, t is 0, and E is phenyl, then any R₇ on E is not an o-alkoxy;
when Q is Q-22, then the compound of formula (I) is selected from the group consisting of 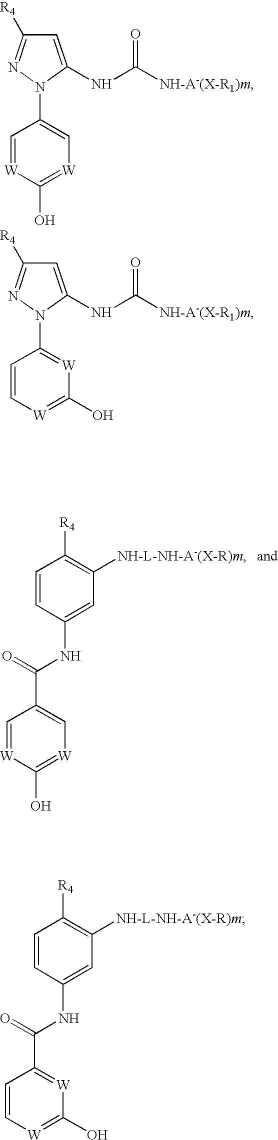
when Q is Q-24, Q-25, Q-26, or Q-31, then the compound of formula (I) is selected from the group consisting of 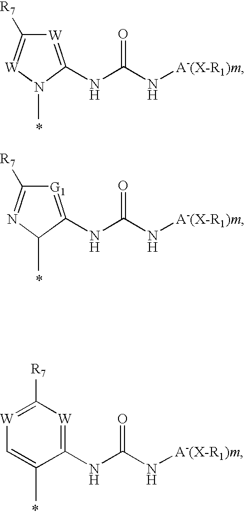
wherein each W is individually selected from the group consisting of —CH— and —N—; each G₁ is individually selected from the group consisting of —O—, —S—, and —N(R₄)—; and *denotes the point of attachment to Q-24, Q-25, Q-26, or Q-31 as follows: 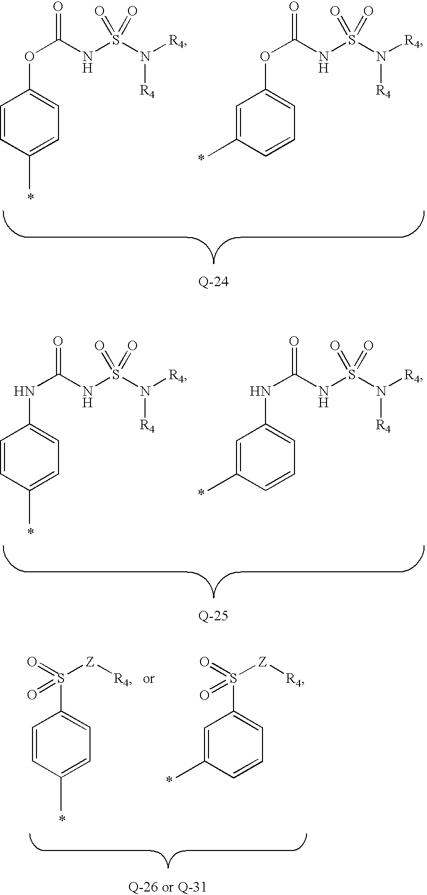
wherein each Z is individually selected from the group consisting of —O— and —N(R₄)—;
When Q is Q-35C as shown the compound of formula (LA) is not

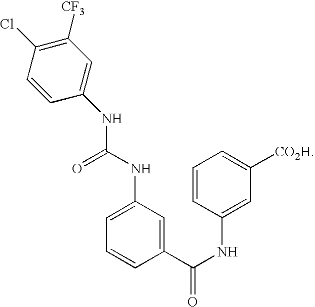

2. The compound of claim 1, wherein R₁ is selected from the group consisting of aryl, 6-5 fused heteroaryls, 6-5 fused heterocyclyls, 5-6 fused heteroaryls, 5-6 fused heterocyclyls, and monocyclic heterocyclyls.

3. The compound of claim 2 wherein R₁ is selected from the group consisting of phenyl, naphthyl, indenyl, indanyl, pyrrolyl, furyl, thienyl, oxazolyl, thiazolyl, isoxazolyl, isothiazolyl, imidazolyl, pyrazolyl, oxadiazolyl, thiadiazolyl, triazolyl, tetrazolyl, pyridinyl, pyrimidinyl, pyrazinyl, pyridazinyl, triazinyl, indolyl, isoindolyl, indazolyl, benzofuranyl, benzothienyl, benzothiazolyl, benzoxazolyl, benzisoxazolyl, benzisothiazolyl, benzimidazolyl, bentriazolyl, imidazopyridinyl, purinyl, phthalimidyl, phthalimidinyl, pyrazinylpyridinyl, pyrimidinopyridinyl, pyrimidinopyrimidinyl, cinnolinyl, quinoxalinyl, quinazolinyl, quinolinyl, isoquinolinyl, phthalazinyl, benzodioxyl, indolinyl, benzisothiazoline-1,1,3-trionyl, dihydroquinolinyl, tetrahydroquinolinyl, dihydroisoquinolyl, tetrahydroisoquinolinyl, benzoazepinyl, benzodiazepinyl, benzoxapinyl, and benzoxazepinyl.

4. The compound of claim 2 wherein R₁ is selected from the group consisting of oxetanyl, azetadinyl, imidazolonyl, tetrahydrofuranyl, pyrrolidinyl, pyrrolinedionyl, pyranyl, thiopyranyl, tetrahydropyranyl, dioxalinyl, piperidinyl, piperidinonyl, morpholinyl, thiomorpholinyl, piperazinyl, piperazinonyl, azepinyl, oxepinyl, and diazepinyl.

5. The compound of claim 1, where R₁ is selected from the group consisting of 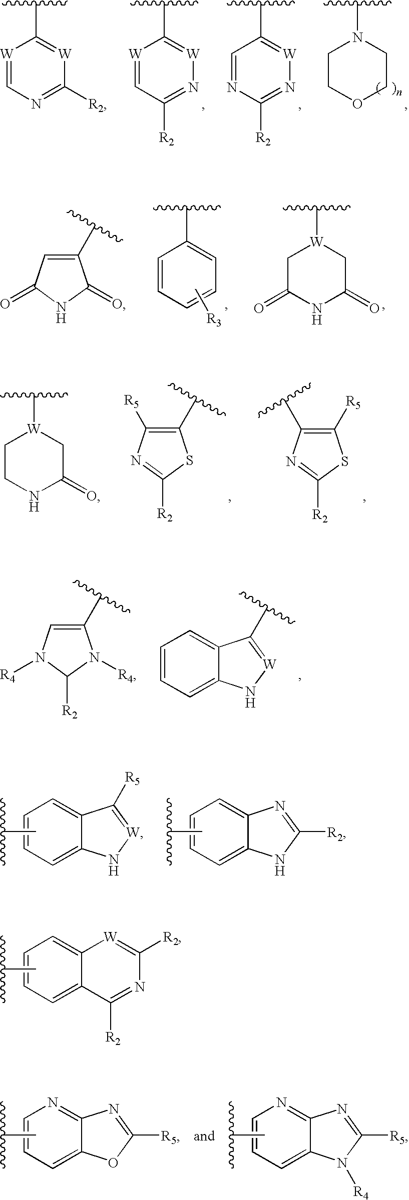
each R₂ is individually selected from the group consisting of —H, alkyls, aminos, alkylaminos, arylaminos, cycloalkylaminos, heterocyclylaminos, halogens, alkoxys, and hydroxys; and
each R₃ is individually selected from the group consisting of —H, alkyls, alkylaminos, arylaminos, cycloalkylaminos, heterocyclylaminos, alkoxys, hydroxys, cyanos, halogens, perfluoroalkyls, alkylsulfinyls, alkylsulfonyls, R₄NHSO₂—, and —NHSO₂R₄.

6. The compound of claim 1, wherein A is selected from the group consisting of aromatic, monocycloheterocyclic, and bicycloheterocyclic rings; and most preferably phenyl, naphthyl, pyridyl, pyrimidyl, thienyl, furyl, pyrrolyl, thiazolyl, isothiazolyl, oxaxolyl, isoxazolyl, imidazolyl, oxadiazolyl, thiadiazolyl, indolyl, indazolyl, benzimidazolyl, benzotriazolyl, isoquinolyl, quinolyl, benzotriazolyl, benzofuranyl, benzothienyl, pyrazolylpyrimidinyl, imidazopyrimidinyl, purinyl, and 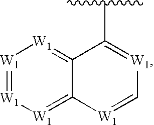
wherein each W₁ is individually selected from the group consisting of —CH— and —N—.

7. The compound of claim 1 of the formula 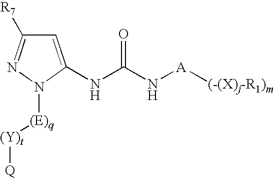
Wherein R7 is taken from the group consisting of t-butyl, CF3, phenyl, or thienyl.

8. The compound of claim 1 of the formula 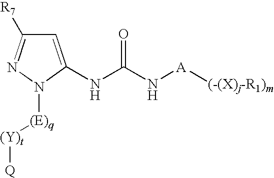
Wherein R7 is taken from the group consisting of halogen-substituted phenyl or C3-C6 carbocyclyl.

9. The compounds of claim 7 of the formula

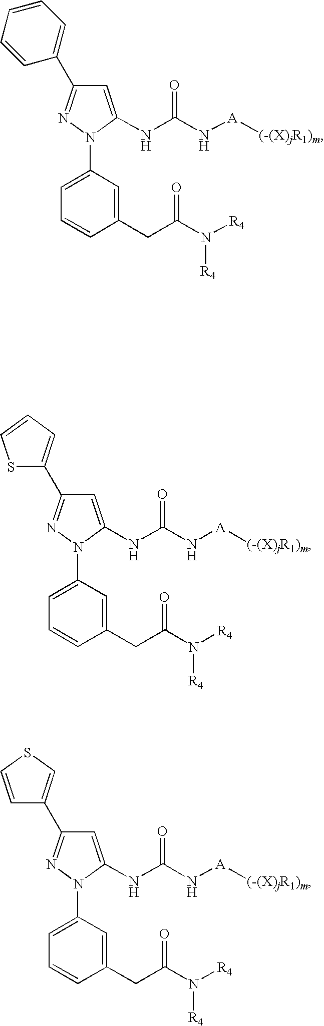

10. The compounds of claim 8 of the formula

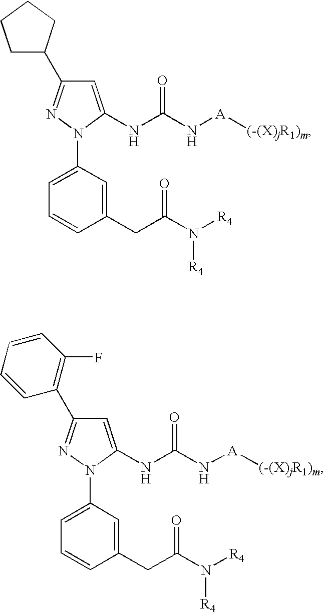

11. The compounds of claim 7, wherein the compound of formula I is taken from 2-(3-(5-(3-(2,3-dichlorophenyl)ureido)-3-phenyl-1H-pyrazol-1-yl)phenyl)acetic acid, 2-(3-(5-(3-(2,3-dichlorophenyl)ureido)-3-(thiophen-2-yl)-1H-pyrazol-1-yl)phenyl)acetic acid, 2-(3-(5-(3-(2,3-dichlorophenyl)ureido) 3-(thiophen-3-yl)-1H-pyrazol-1-yl)phenyl)acetic acid, 2-(4-(5-(3-(2,3-dichlorophenyl)ureido)-3-phenyl-1H-pyrazol-1-yl)phenyl)acetic acid, 2-(4-(5-(3-(2,3-dichlorophenyl)ureido)-3-(thiophen-2-yl)-1H-pyrazol-1-yl)phenyl)acetic acid, 2-(4-(5-(3-(2,3-dichlorophenyl)ureido)-3-(thiophen-2-yl)-1H-pyrazol-1-yl)phenyl)propanoic acid, 2-(4-(5-(3-(2,3-dichlorophenyl)ureido)-3-(thiophen-3-yl)-1H-pyrazol-1-yl)phenyl)acetic acid, methyl 2-(4-(5-(3-(2-(3-dichlorophenyl)ureido)-3-(thiophen-3-yl)-1H-pyrazol-1-yl)phenylacetate, 1-(1-(3-(2-amino-2-oxoethyl)phenyl)-3-phenyl-1H-pyrazol-5-yl)-3-(2,3-dichlorophenyl)urea, 1-(1-(3-(2-amino-2-oxoethyl)phenyl)-3-(thiophen-2-yl)-1H-pyrazol-5-yl)-3-(2,3-dichlorophenyl)urea, 1-(1-(3-(2-amino-2-oxoethyl)phenyl)-3-(thiophen-3-yl)-1H-pyrazol-5-yl)-3-(2,3-dichlorophenyl)urea, 1-(1 (1-(3-(1-amino-1-oxopropan-2-yl)phenyl)-3-(thiophen-3-yl)-1H-pyrazol-5-yl)-3-(2,3-dichlorophenyl)urea, 1-(2,3-dichlorophenyl)-3-(1-(3-(2-(2-hydroxy ethylamino)-2-oxoethyl)phenyl)-3-phenyl-1H-pyrazol-5-yl)urea, 1-(2,3-dichlorophenyl)-3-(1-(3-(2-(2-hydroxyethylamino)-2-oxoethyl)phenyl)-3-(thiophen-2-yl)-1H-pyrazol-5-yl)urea, 1-(2,3-dichlorophenyl)-3-(1-(3-(2-(2-hydroxyethylamino)-2-oxoethyl)phenyl)-3-(thiophen-3-yl)-1H-pyrazol-5-yl)urea, 1-(2,3-dichlorophenyl)-3-(1-(3-(2-(2,3-dihydroxypropylamino)-2-oxoethyl)phenyl)-3-(thiophen-2-2-yl)-1H-pyrazol-5-yl)urea, 1-(2,3-dichlorophenyl)-3-(1-(3-(2-(1,3-dihydroxypropan-2-ylamino)-2-oxoethyl)phenyl)-3-(thiophen-2-yl)-1H-pyrazol-5-yl)urea, 1-(2,3-dichlorophenyl)-3-(1-(3-(2-((S)-3-hydroxypyrrolidin-1-yl)-2-oxoethyl)phenyl)-3-(thiophen-2-yl)-1H-pyrazol-5-yl)urea, 1-(3-tert-butyl-1-(3-(2-((R)-3-(dimethylamino)pyrrolidin-1-yl)-2-oxoethyl)phenyl)-1H-pyrozol-5-yl)-3-(2,3-dichlorophenyl)urea, 1-(3-tert-butyl-1-(3-(2-((S)-3-(dimethylamino)pyrrolidin-1-yl)-2-oxoethyl)phenyl)-1H-pyrazol-5-yl)-3-(2,3-dichlorophenyl)urea, 1-(1-(4-(2-amino-2-oxoethyl)phenyl)-3-(thiophen-2-yl)-1H-pyrazol-5-yl)-3-(2,3-dichlorophenyl)urea, 1-(1-(4-(2-amino-2-oxoethyl)phenyl)-3-(thiophen-3-yl)-1H-pyrazol-5-yl)-3-(2,3-dichlorophenyl)urea, 1-(2,3-dichlorophenyl)-3-(1-(4-(2-(2-hydroxyethylamino)-2-oxoethyl)phenyl)-3-phenyl-1H-pyrazol-5-yl)urea, 1-(2,3-dichlorophenyl)-3-(1-(4-(2-(2,3-dihydroxypropylamino)-2-oxoethyl)phenyl)-3-(thiophen-2-yl)-1H-pyrazol-5-yl)urea, 1-(2,3-dichlorophenyl)-3-(1-(4-(2-(1,3-dihydroxypropan-2-ylamino)-2-oxoethyl)phenyl)-3-(thiophen-2-yl)-1H-pyrazol-5-yl)urea, 1-(2,3-dichlorophenyl)-3-(1-(4-(2-(4-methylpiperazin-1-yl)-2-oxoethyl)-phenyl)-3-phenyl-1H-pyrazol-5-yl)urea, 1-(2-(4-(3-tert-butyl-5-(3-(2,3-dichlorophenyl)ureido)-1H-pyrazol-1-yl)phenyl)acetyl)piperidine-3-carboxylic acid, (R)-1-(2,3-dichlorophenyl)-3-(1-(4-(2-(2-((hydroxymethyl)pyrrolidin-1-yl)-2-oxoethyl)phenyl)-3-phenyl-1H-pyrazol-5-yl)urea, (S)-1-(3-tert-butyl-1-(4-(2-(3-hydroxypyrrolidin-1-yl)-2-oxoethyl)phenyl)-1H-pyrazol-5-yl)-3-(2,3-dichlorophenyl)urea, (R)-1-(3-tert-butyl-1-(4-(2-(3-(dimethylamino)pyrrolidin-1-yl)-2-oxoethyl)phenyl)-1H-pyrazol-5-yl)-3-(2,3-dichlorophenyl)urea, (R)-1-(3-tert-butyl-1-(4-(2-(3-(dimethylamino)pyrrolidin-1-yl)-2-oxoethyl)phenyl)-1H-pyrazol-5-yl)3-(2,3-dichlorophenyl)urea, 1-(3-tert-butyl-1-(3-((2,4,5-trioxoimidazolidin-1-yl)methyl)phenyl)-1H-pyrazol-5-yl)-3-(2,3-dichlorophenyl)urea, 1-(2,3-dichlorophenyl)-3-(1-(3-(hydroxymethyl)phenyl)-3-phenyl-1H-pyrazol-5-yl)urea, 1-(2,3-dichlorophenyl)-3-(1-(3-(1-(hydroxymethylphenyl)-3-(thiophen-2-yl)-1H-pyrazol-5-yl)urea, 3-(3-(5-(3-(2,3-dichlorophenyl)ureido)-3-(thiophen-2-yl)-1H-pyrazol-1-yl)phenyl)-2-methylpropanoic acid, 1-(1-(3-(2-amino-2-oxoetheylphenyl)-3-tert-butyl-1H-pyrazol-5-yl)-3-(2,3,4-trifluorophenyl)urea, 2-(4-(3-tert-butyl-5-(3-(2,3,4-trifluorophenyl)ureido)-1H-pyrazol-1-yl)phenyl)acetic acid, 1-(1-(3-(hydroxymethyl)phenyl)-3-(thiophen-2-yl)-1H-pyrazol-5-yl)-3-(2,3,4-trifluorophenyl)urea, 1-(1-(3-(2-amino-2-oxoethyl)phenyl)-3-tert-butyl-1H-pyrazol-5-yl)-3-(2,4,5-trifluorophenyl)urea, 1-(1-(3-(2-amino-2-oxoethyl)phenyl)-3-tert-butyl-1H-pyrazol-5-yl)-3-(2,3-difluorophenyl)urea, 1-(1-(3-(2-amino-2-oxoethyl)phenyl)-3-tert-butyl-1H-pyrazol-5-yl)-3-(2,4-difluorophenyl)urea, 2-(4-(3-tert-butyl-5-(3-(2,4-difluorophenyl)ureido)-1H-pyrazol-1-yl)phenyl)acetic acid, 1-(1-(4-(2-amino-2-oxoethyl)phenyl)-3-tert-butyl-1H-pyrazol-5-yl)-3-(2,4-difluorophenyl)urea, 1-(3-tert-butyl-1-(3-cyanophenyl)-1H-pyrazol-5-yl)-3-(2,4-difluorophenyl)urea, 1-(1-(3-(2-amino-2-oxoethyl)phenyl)-3-tert-butyl-1H-pyrazol-5-yl)-3-(3-(pyridin-3-yloxy)phenyl)urea, 1-(1-(3-(2-amino-2-oxoethyl)phenyl)-3-phenyl-1H-pyrazol-5-yl)-3-(3-(pyridin-3-yloxy)phenyl)urea, 1-(1-(3-(2-amino-2-oxoethyl)phenyl)-3-(thiophen-2-yl)-1H-pyrazol-5-yl)-3-(3-(pyridin-3-yloxy)phenyl)urea, 1-(1-(3-(2-amino-2-oxoethyl)phenyl)-3-(trifluoromethyl)-1H-pyrazol-5-yl)-3-(3-(pyridin-3-yloxy)phenyl)urea, 1-(1-(3-(2-amino-2-oxoethyl)phenyl)-3-tert-butyl-1H-pyrazol-5-yl)-3-(3-(pyrazin-2-yloxy)phenyl)urea, 1-(1-(3-(2-amino-2-oxoethyl)phenyl)-3-tert-butyl-1H-pyrazol-5-yl)-3-(4-(pyridin-4-yloxy)phenyl)urea, 1-(1-(3-(2-amino-2-oxoethyl)phenyl)-3-tert-butyl-1H-pyrazol-5-yl)-3-(4-(2-(methylcarbamoyl)pyridin-4-yloxy)phenyl)urea, 1-(1-(3-(2-amino-2-oxoethyl)phenyl)-3-tert-butyl-1H-pyrazol-5-yl)-3-(3-(pyridin-3-yl)phenyl)urea, 1-(1-(3-(2-amino-2-oxoethyl)phenyl)-3-tert-butyl-1H-pyrazol-5-yl)-3-(3-(6-aminopyridin-3-yl)phenyl)urea, 1-(1-(3-(2-amino-2-oxoethyl)phenyl)-3-tert-butyl-1H-pyrazol-5-yl)-3-(3-(pyrazin-2-yl)phenyl)urea, 1-(1-(3-(2-amino-2-oxoethyl)phenyl)-3-tert-butyl-1H-pyrazol-5-yl)-3-(4-(1-oxoisoindolin-4-yl)phenyl)urea, 1-(1-(3-(2-amino-2-oxoethyl)phenyl)-3-tert-butyl-1H-pyrazol-5-yl)-3-(3-(8-methyl-7-oxo-7,8-dihydropyrido[2,3-d]pyrimidin-6-yl)phenyl)urea, 1-(1-(3-(2-amino-2-oxoethyl)phenyl)-3-tert-butyl-1H-pyrazol-5-yl)-3-(4-methyl-3-(pyrimidin-2-ylamino)phenyl)urea, 1-(1-(3-(2-amino-2-oxoethyl)phenyl)-3-tert-butyl-1H-pyrazol-5-yl)-3-(4-methyl-3-(4-(pyridin-3-yl)pyrimidin-2-ylamino)phenyl)urea, and 1-(3-tert-butyl-1-(3-(2-(2,3-dihydroxypropylamino)-2-oxoethyl)phenyl)-1H-pyrazol-5-yl)-3-(4-methyl-3-(4-(pyridin-3-yl)pyrimidin-2-ylamino)phenyl)urea.

12. The compounds of claim 8, wherein the compound of formula I is taken from 2-(3-(5-(3-(2,3-dichlorophenyl)ureido)-3-(4-fluorophenyl)-1H-pyrazol-1-yl)phenyl)acetic acid, 2-(3-(5-(3-(2,3-dichlorophenyl)ureido)-3-(3-fluorophenyl)-1H-pyrazol-1-yl)phenyl)acetic acid, 2-(3-(5-(3-(2,3-dichlorophenyl)ureido)-3-(2-fluorophenyl)-1H-pyrazol-1-yl)phenyl)acetic acid, 2-(3-(3-cyclopentyl-5-(3-(2,3-dichlorophenyl)ureido)-1H-pyrazol-1-yl)phenyl)acetic acid, ethyl 2-(4-(3-cyclopentyl-5-(3-(2,3-dichlorophenyl)ureido)-1H-pyrazol-1-yl)phenyl)acetate, 2-(4-(5-(3-(2,3-dichlorophenyl)ureido)-3-(3-fluorophenyl)-1H-pyrazol-1-yl)phenyl)acetic acid, 2-(4-(5-(3-(2,3-dichlorophenyl)ureido)-3-(2-fluorophenyl)-1H-pyrazol-1-yl)phenyl)acetic acid, 2-(4-(3-cyclopentyl-5-(3-(2,3-dichlorophenyl)ureido)-1H-pyrazol-1-yl)phenyl)acetic acid, 1-(1-(3-(2-amino-2-oxoethyl)phenyl)-3-cyclopentyl-1H-pyrazol-5-yl)-3-(2,3-dichlorophenyl)urea, 1-(1-(3-(2-amino-2-oxoethyl)phenyl)-3-(4-fluorophenyl)-1H-pyrazol-5-yl)-3-(2,3-dichlorophenyl)urea, 1-(1-(3-(2-amino-2-oxoethyl)phenyl)-3-(3-fluorophenyl)-1H-pyrazol-5-yl)-3-(2,3-dichlorophenyl)urea, 1-(1-(3-(2-amino-2-oxoethyl)phenyl)-3-(3-fluorophenyl)-1H-pyrazol-5-yl)-3-(2,3-dichlorophenyl)urea, 1-(2,3-dichlorophenyl)-3-(3-(3-fluorophenyl)-1-(3-(2-(2-hydroxyethylamino)-2-oxoethyl)phenyl)-1H-pyrazol-5-yl)urea, 1-(2,3-dichlorophenyl)-3-(3-(2-fluorophenyl)-1-(3-(2-(2-hydroxyethylamino)-2-oxoethyl)phenyl)-1H-pyrazol-5-yl)urea, 1-(2,3-dichlorophenyl)-3-(1-(3-(2-(2,3-dihydroxypropylamino)-2-oxoethyl)phenyl)-3-(2-fluorophenyl)-1H-pyrazol-5-yl)urea 1-(2,3-dichlorophenyl)-3-(1-(3-(2-(1,3-dihydroxypropan-2-ylamino)-2-oxoethyl)phenyl)-3-(2-fluorophenyl)-1H-pyrazol-5-yl)urea, 1-(1-(4-(2-amino-2-oxoethyl)phenyl)-3-(2-fluorophenyl)-1H-pyrazol-5-yl)-3-(2,3-dichlorophenyl)urea, 1-(1-(4-(2-amino-2-oxoethyl)phenyl)-3-cyclopentyl-1H-pyrazol-5-yl)-3-(2,3-dichlorophenyl)urea, 1-(2,3-dichlorophenyl)-3-(1-(4-(2-(2,3-dihydroxypropylamino)-2-oxoethyl)phenyl)-3-(2-fluorophenyl)-1H-pyrazol-5-yl)urea, 1-(2,3-dichlorophenyl)-3-(1-(4-(2-(1,3-dihydroxypropan-2-ylamino)-2-oxoethyl)phenyl)-3-(2-fluorophenyl)-1H-pyrazol-5-yl)urea, 1-(2,3-dichlorophenyl)-3-(3-(2-fluorophenyl)-1-(4-(2-((S)-3-hydroxypyrrolidin-1-yl)-2-oxoethyl)phenyl)-1H-pyrazol-5-yl)urea, 1-(2,3-dichlorophenyl)-3-(3-(2-fluorophenyl)-1-(3-(hydroxymethyl)phenyl)-1H-pyrazol-5-yl)urea, 1-(3-cyclopentyl-1-(3-(2-(2,3-dihydroxypropylamino)-2-oxoethyl)phenyl)-1H-pyrazol-5-yl)-3-(2,3-dichlorophenyl)urea, 1-(3-cyclopentyl-1-(3-(2-(2-hydroxyethylamino)-2-oxoethyl)phenyl)-1H-pyrazol-5-yl)-3-(2,3-dichlorophenyl)urea, 1-(1-(3-(3-amino-2-methyl-3-oxopropyl)phenyl)-3-(2-fluorophenyl)-1H-pyrazol-5-yl)-3-(2,3-dichlorophenyl)urea, 1-(1-(3-(2-amino-2-oxoethyl)phenyl)-3-cyclopentyl-1H-pyrazol-5-yl)-3-(4-(1-oxoisoindolin-4-yl)phenyl)urea, and 1-(1-(3-(2-amino-2-oxoethyl)phenyl)-3-cyclopentyl-1H-pyrazol-5-yl)-3-(3-(8-methyl-7-oxo-7,8-dihydropyrido[2,3-d]pyrimidin-6-yl)phenyl)urea.

13. The compounds of claim 1, wherein m is 1 and R₁ is taken from the group consisting of phenyl, naphthyl, indenyl, indanyl, pyrrolyl, furyl, thienyl, oxazolyl, thiazolyl, isoxazolyl, isothiazolyl, imidazolyl, pyrazolyl, oxadiazolyl, thiadiazolyl, triazolyl, tetrazolyl, pyridinyl, pyrimidinyl, pyrazinyl, pyridazinyl, triazinyl, indolyl, isoindolyl, indazolyl, benzofuranyl, benzothienyl, benzothiazolyl, benzoxazolyl, benzisoxazolyl, benzisothiazolyl, benzimidazolyl, bentriazolyl, imidazopyridinyl, purinyl, phthalimidyl, phthalimidinyl, pyrazinylpyridinyl, pyrimidinopyridinyl, pyrimidinopyrimidinyl, cinnolinyl, quinoxalinyl, quinazolinyl, quinolinyl, isoquinolinyl, phthalazinyl, benzodioxoyl, indolinyl, benzisothiazolone-1,1,3-trionyl, dihydroquinolinyl, tetrahydroquinolinyl, dihydroisoquinolyl, tetrahydroisoquinolinyl, benzoazepinyl, benzodiazepinyl, benzoxapinyl, and benzoxazepinyl.

14. Compounds of claim 1 of the formulae

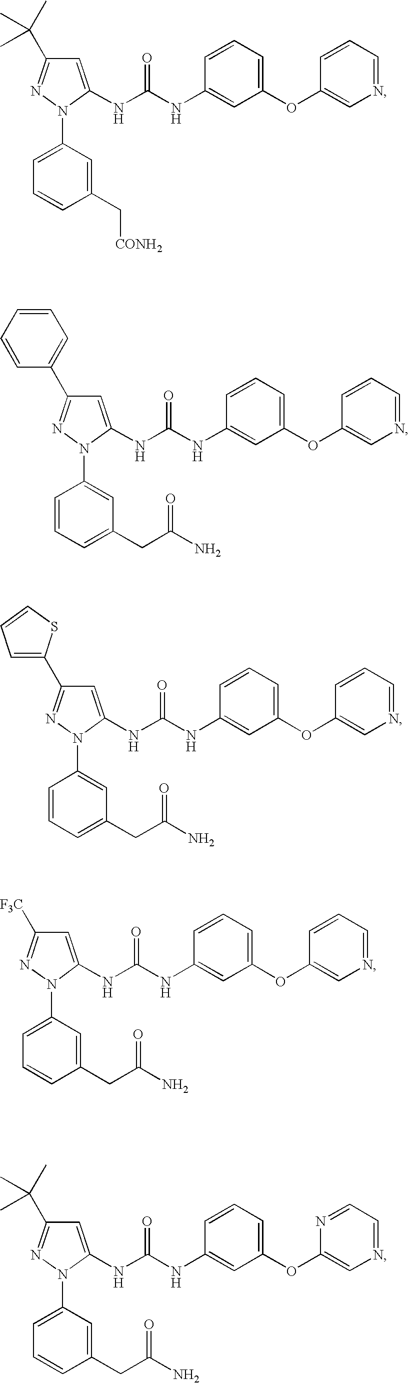

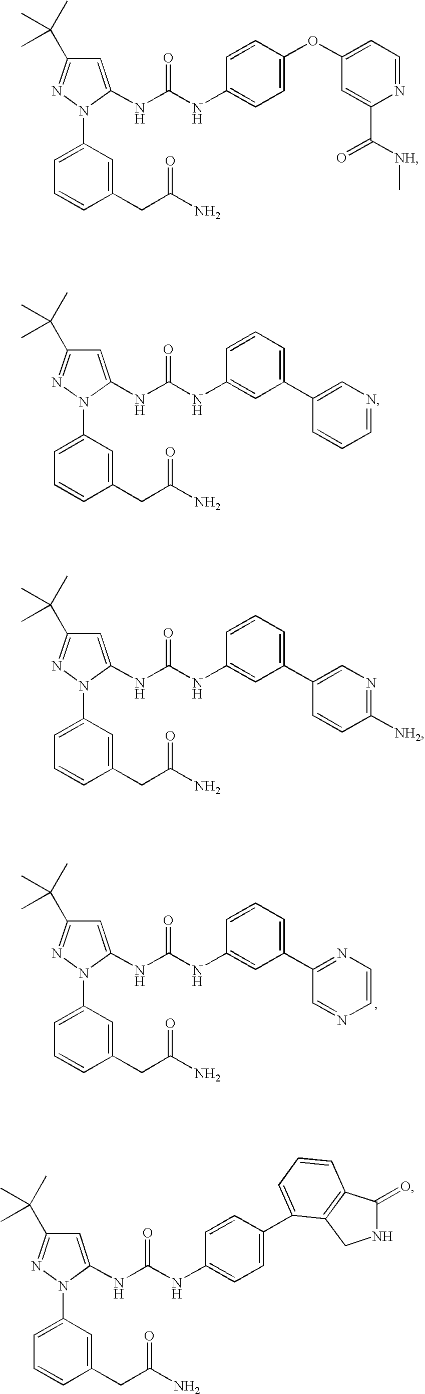

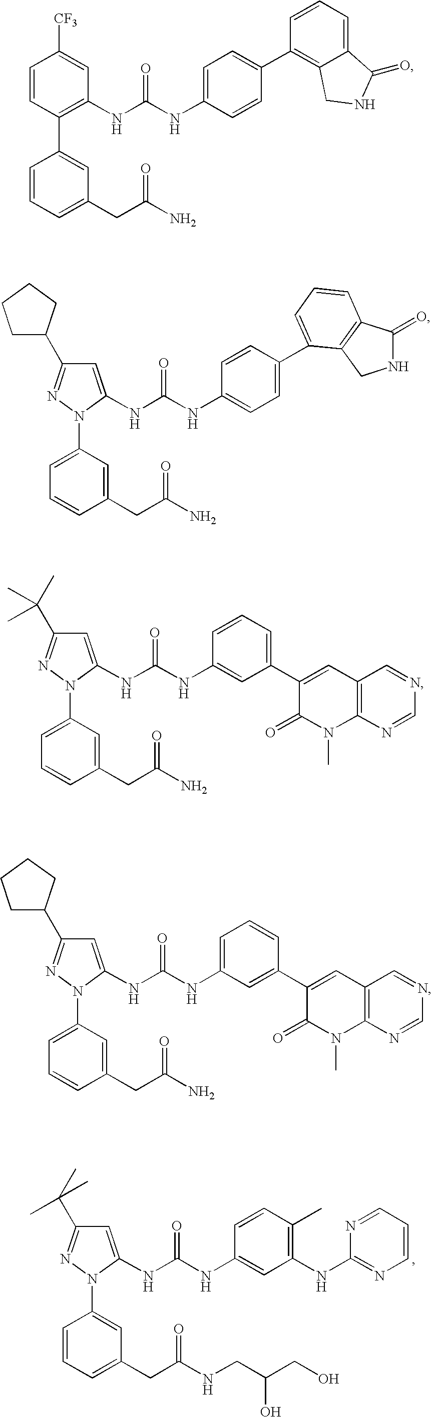

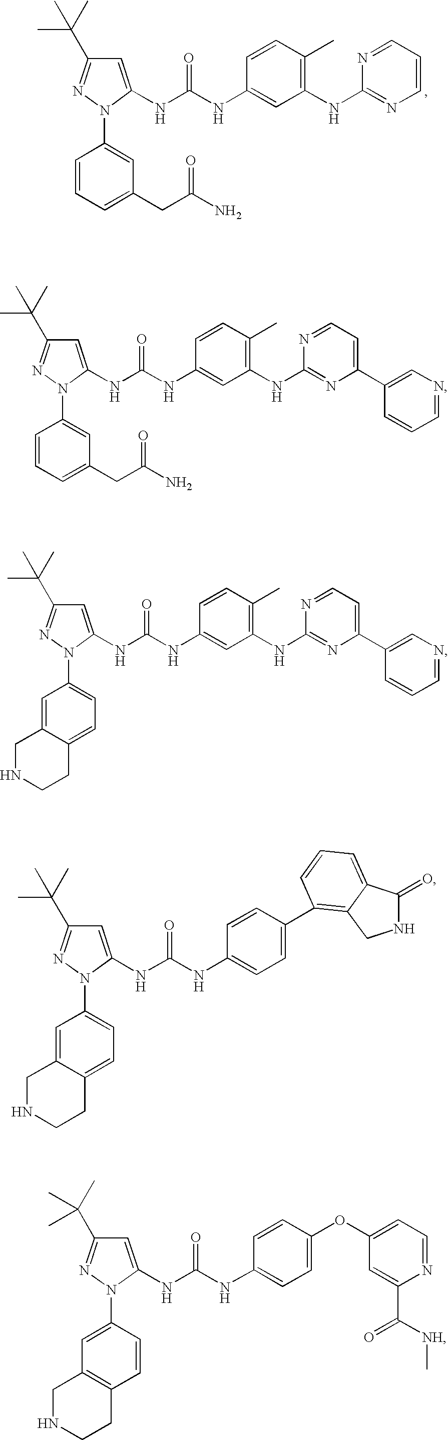

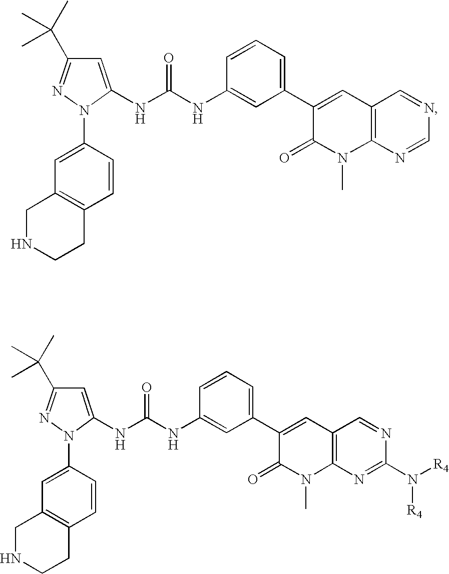

15. A method of modulating the activation state of a kinase comprising the step of contacting said kinase with a molecule of claim 1.

16. The method of claim 15, said contacting step occurring at the region of a switch control pocket of said kinase.

17. The method of claim 16, said switch control pocket of said kinase comprising an amino acid residue sequence operable for binding to said compound.

18. The method of claim 16, said switch control pocket selected from the group consisting of simple, composite and combined switch control pockets.

19. The method of claim 18, said region being selected from the group consisting of the α-C helix, the α-D helix, the catalytic loop, the switch control ligand sequence, and the C-lobe residues and combinations thereof.

20. The method of claim 19, said kinase being p38-alpha kinase and the α-C helix region thereof includes SEQ ID NO. 2.

21. The method of claim 19, said kinase being p38-alpha kinase and the catalytic loop region thereof includes SEQ ID NO. 3.

22. The method of claim 19, said kinase being p38-alpha kinase and the switch control ligand region thereof includes SEQ ID NO. 4, SEQ ID NO. 5, and combinations thereof.

23. The method of claim 19, said kinase being p38-alpha kinase and the C-lobe region thereof includes SEQ ID NO. 6.

24. The method of claim 15, said kinase selected from the group consisting of consensus wild type, disease polymorphs, and fusion proteins of serine-threonine kinases, tyrosine kinases, receptor tyrosine kinases, and mixed function kinases.

25. The method of claim 15, said activation state being selected from the group consisting of the upregulated and downregulated states.

26. The method of claim 15, said molecule being an antagonist of the on switch control pocket for said kinase.

27. The method of claim 15, said molecule being an agonist of the off switch control pocket for said kinase.

28. The method of claim 15, said method including the step of administering said molecule to an individual undergoing treatment for a condition selected from the group consisting of human inflammation, rheumatoid arthritis, rheumatoid spondylitis, ostero-arthritis, asthma, gouty arthritis, sepsis, septic shock, endotoxic shock, Gram-negative sepsis, toxic shock syndrome, adult respiratory distress syndrome, stroke, reperfusion injury, neural trauma, neural ischemia, psoriasis, restenosis, chronic pulmonary inflammatory disease, bone resorptive diseases, graft-versus-host reaction, Chron's disease, ulcerative colitis, inflammatory bowel disease, pyresis, and combinations thereof.

29. The method of treating an individual suffering from a condition selected from the group consisting of human inflammation, rheumatoid arthritis) rheumatoid spondylitis, ostero-arthritis, asthma, gouty arthritis, sepsis, septic shock, endotoxic shock, Gram-negative sepsis, toxic shock syndrome, adult respiratory distress syndrome, stroke, reperfusion injury, neural trauma, neural ischemia, psoriasis, restenosis, chronic pulmonary inflammatory disease, bone resorptive diseases, graft-versus-host reaction, Chron's disease, ulcerative colitis, inflammatory bowel disease, pyresis, and combinations thereof, said method comprising the step of administering to said individual a compound as set forth in claim 11.

30. The method of treating an individual suffering from a condition selected from the group consisting of human inflammation, rheumatoid arthritis, rheumatoid spondylitis, ostero-arthritis, asthma, gouty arthritis, sepsis, septic shock, endotoxic shock, Gram-negative sepsis, toxic shock syndrome, adult respiratory distress syndrome, stroke, reperfusion injury, neural trauma, neural ischemia, psoriasis, restenosis, chronic pulmonary inflammatory disease, bone resorptive diseases, graft-versus-host reaction, Chron's disease, ulcerative colitis, inflammatory bowel disease, pyresis, and combinations thereof, said method comprising the step of administering to said individual a compound as set forth in claim 12.

31. The method of claim 28, 29 or 30, said molecule being administered by a method selected from the group consisting of oral, parenteral, inhalation, and subcutaneous.

32. The method of claim 28, 29, or 30, said kinase being p-38 alpha kinase.

33-43. (canceled)

44. The method of claim 15, wherein the kinase is selected from the group consisting of abl kinase, Bcr-abl kinase, Braf kinase, VEGFR kinase, PDGFR kinase, fusion proteins of any of the foregoing kinases, and disease polymorphs of any of the foregoing kinases.

45. The method of treating an individual suffering from a condition selected from the group consisting of cancer, hyperproliferative diseases, diseases characterized by hyper-vascularization including diabetic retinopathy and macular degeneration, and combinations thereof, said method comprising the step of administering to said individual a compound as set forth in claim 1.

46. The method of treating an individual suffering from a condition selected from the group consisting of cancer, hyperproliferative diseases, diseases characterized by hyper-vascularization including diabetic retinopathy and macular degeneration, and combinations thereof, said method comprising the step of administering to said individual a compound as set forth in claim 13.

47. The method of claim 45 or 46, said compound being administered by a method selected from the group consisting of oral, parenteral, inhalation, and subcutaneous.
